(19) United States
(12) Patent Application Publication
(10) Pub. No.: US 2026/0327586 A1
(43) Pub. Date: Oct. 22, 2026

(54) COMPOSITIONS AND METHODS FOR MULTIPLEX EPIGENETIC REGULATION

(71) Applicant: nChroma Bio, Inc., Boston, MA (US)

(72) Inventors: Jamie Lynn Schafer, Boston, MA (US); Aron Brandon Jaffe, Brookline, MA (US); Morgan Maeder, Waban, MA (US); Ari Friedland, Cambridge, MA (US); Vic Myer, Arlington, MA (US)

(73) Assignee: nChroma Bio, Inc., Boston, MA (US)

(21) Appl. No.: 19/483,778

(22) PCT Filed: May 15, 2024
(86) PCT No.: PCT/US2024/029517

§ 371 Date: Nov. 12, 2025

Related U.S. Application Data
(60) Provisional application No. 63/502,400, filed on May 15, 2023.
(60) Provisional application No. 63/579,806, filed on Aug. 30, 2023.
(60) Provisional application No. 63/598,526, filed on Nov. 13, 2023.
(60) Provisional application No. 63/606,081, filed on Dec. 4, 2023.
(60) Provisional application No. 63/614,451, filed on Dec. 22, 2023.

(51) Int. Cl. C12N 9/22; C07K 14/705; C12N 5/0783; C12N 9/10; C12N 15/11; C12N 15/86

(52) U.S. Cl. CPC C12N 9/226; C07K 14/70539; C12N 5/0636; C12N 9/1007; C12N 15/111; C12N 15/86; C07K 2319/81; C12N 2310/20; C12N 2510/00; C12N 2740/15043; C12Y 201/01037

(57) ABSTRACT

This disclosure relates to compositions and methods comprising epigenetic editors for multiplex epigenetic modification, as well as nucleic acids and vectors encoding the same. Also disclosed are cells epigenetically modified by the epigenetic editors.

### CROSS-REFERENCE TO RELATED APPLICATIONS

This application claims the benefit under 35 U.S.C. § 119(e) of U.S. Provisional Application No. 63/502,400, filed May 15, 2023, entitled “COMPOSITIONS AND METHODS FOR MULTIPLEX EPIGENETIC REGULATION,” U.S. Provisional Application No. 63/579,806, filed Aug. 30, 2023, entitled “COMPOSITIONS AND METHODS FOR MULTIPLEX EPIGENETIC REGULATION,” U.S. Provisional Application No. 63/598,526, filed Nov. 13, 2023, entitled “COMPOSITIONS AND METHODS FOR MULTIPLEX EPIGENETIC REGULATION,” U.S. Provisional Application No. 63/606,081, filed Dec. 4, 2023, entitled “COMPOSITIONS AND METHODS FOR MULTIPLEX EPIGENETIC REGULATION,” U.S. Provisional Application No. 63/614,451, filed Dec. 22, 2023, entitled “COMPOSITIONS AND METHODS FOR MULTIPLEX EPIGENETIC REGULATION,” the disclosure of each which is hereby incorporated by reference in its entirety.

### REFERENCE TO AN ELECTRONIC SEQUENCE LISTING

The contents of the electronic sequence listing (C169870038WO00-SEQ-AXW.xml; Size: 2,085,869 bytes; and Date of Creation: May 14, 2024) is herein incorporated by reference in its entirety.

### BACKGROUND

Adoptive cell therapy using genetically engineered immune cells has emerged as a promising approach to treat cancer, infections, autoimmune diseases, and other disorders. In many scenarios, engineered immune cells comprising a plurality of modifications, e.g., a plurality of silenced genes, are advantageous. However, traditional genetic engineering strategies typically rely on permanent manipulation of cells at the genomic level, and typically involve cutting the genomic DNA in order to effect the desired mutations, which is associated with significant risks, including, for example, chromosomal translocations, undesired mutations (e.g., insertions and/or deletions of nucleotides) at the targeted site, and off-target mutations. There remains a need for efficient and safe methods of genetically engineering immune cells.

### SUMMARY

The present disclosure provides systems and compositions for multiplex epigenetic modification (“epigenetic editors” or “epigenetic editing systems” herein), and methods of using the same to generate epigenetic modification at two or more target sites, e.g., at two or more target genes, including in host cells and organisms.

In some aspects, the present disclosure provides a system for repressing transcription of two or more human genes in a human cell, optionally a human T lymphocyte or a human NK cell, comprising (a) two or more sets of fusion proteins comprising (i) a first fusion protein that collectively comprises a first DNA methyltransferase (DNMT) domain and/or a first domain that recruits a DNMT (first recruiter domain), optionally wherein the first DNMT domain and/or the first recruiter domain comprise a first DNMT3A domain and/or a first DNMT3L domain, and optionally wherein the DNMT recruited by the first recruiter domain is DNMT3A, and a first transcriptional repressor domain, each of the first DNMT domain and/or the first recruiter domain, and the first transcriptional repressor domain being linked to a first DNA-binding domain that binds to a target region in one or more human genes selected from B2M, CIITA, TRAC, CD247, RFX5, RFXAP, CD3D, and CD3G, or a cis regulatory element regulating one or more of B2M, CIITA, TRAC, CD247, RFX5, RFXAP, CD3D, and CD3G, or one or more nucleic acid molecules encoding the first fusion protein, (ii) a second fusion protein that collectively comprises a second DNA methyltransferase (DNMT) domain and/or a second domain that recruits a DNMT (second recruiter domain), optionally wherein the second DNMT domain and/or the second recruiter domain comprise a second DNMT3A domain and/or a second DNMT3L domain, and optionally wherein the recruited second DNMT is DNMT3A, and a second transcriptional repressor domain, each of the second DNMT domain and/or the second recruiter domain, and the second transcriptional repressor domain being linked to a second DNA-binding domain that binds to a target region in one or more human genes selected from B2M, CIITA, TRAC, CD247, RFX5, RFXAP, CD3D, and CD3G, or a cis regulatory element regulating one or more of B2M, CIITA, TRAC, CD247, RFX5, RFXAP, CD3D, and CD3G, or one or more nucleic acid molecules encoding the second fusion protein, wherein the system does not generate a DNA break in the B2M, CIITA, TRAC, CD247, RFX5, RFXAP, CD3D, or CD3G gene.

In some embodiments, the first and/or the second DNA-binding domain each comprise a dead CRISPR Cas (dCas) domain, a ZFP domain, or a TALE domain. In some embodiments, the first and/or the second DNA-binding domain comprises a dCas9 domain and the system further comprises (i) one or more guide RNAs or (ii) nucleic acid molecules coding for the one or more guide RNAs.

In some aspects, the present disclosure provides a system for repressing transcription of two or more human genes in a human cell, optionally a human T lymphocyte or a human NK cell, comprising (a) a first fusion protein comprising a first DNMT3A domain, a first DNMT3L domain, a first DNA-binding domain, and a first transcriptional repressor domain, and (b) a second fusion protein comprising a second DNMT3A domain, a second DNMT3L domain, a second DNA-binding domain, and a second transcriptional repressor domain, or (c) nucleic acid molecules encoding the first fusion protein and the second fusion protein, wherein the system does not generate a DNA break in the two or more genes bound by the first DNA-binding domain and the second DNA-binding domain.

In some embodiments, the first and/or the second DNA-binding domain comprises a dead CRISPR Cas (dCas) domain, a ZFP domain, or a TALE domain. In some embodiments, the first and/or the second DNA-binding domain comprises a dCas9 domain and the system further comprises (i) two or more guide RNAs or (ii) nucleic acid molecules coding for the two or more guide RNAs.

In some embodiments, the first and/or the second dCas domain comprises a dCas9 sequence, optionally a sequence with at least 90% identity to SEQ ID NO: 12 or 13.

In some embodiments, the first and/or the second DNA-binding domain binds to a target sequence in SEQ ID NO: 1219, 1220, 1283, 1284, 1285, 1286, 1287, 1288, 1313, or 1314.

In some embodiments, the first and/or the second ZFP domain targets a nucleotide sequence selected from SEQ ID NOs: 700-740, 741-795, or 796-856.

In some embodiments, the first and/or the second DNMT3A domain comprises a sequence with at least 90% identity to SEQ ID NO: 574 or 575. In some embodiments, the first and/or the second DNMT3L domain comprises a sequence with at least 90% identity to a sequence selected from SEQ ID NOs: 578-581. In some embodiments, the first and/or the second DNMT3L domain comprises a sequence with at least 90% identity to a sequence selected from SEQ ID NOs: 582-603. In some embodiments, the first and/or the second DNMT domain comprises a sequence with at least 90% identity to a sequence selected from SEQ ID NOs: 601-603.

In some embodiments, the first and/or the second transcriptional repressor domain comprises a sequence with at least 90% identity to a sequence selected from SEQ ID NOs: 33-570. In some embodiments, the first and/or the second transcriptional repressor domain comprises a KRAB domain derived from KOX1, ZIM3, ZFP28, or ZN627. In some embodiments, the KRAB domain comprises a sequence with at least 90% identity to a sequence selected from SEQ ID NOs: 89, 116, 245, and 255. In some embodiments, the first and/or the second transcriptional repressor domain comprises a fusion of the N- and C-terminal regions of ZIM3 and KOX1 KRAB, and optionally comprises the amino acid sequence of SEQ ID NO: 571 or 572. In some embodiments, the first and/or the second transcriptional repressor domain is derived from KAP1, MECP2, HP1a/CBX5, HP1b, CBX8, CDYL2, TOX, TOX3, TOX4, EED, EZH2, RBBP4, RCOR1, or SCML2.

In some aspects, the present disclosure provides a system for repressing transcription of two or more human genes in a human cell, optionally a human T lymphocyte or a human NK cell, comprising a) a fusion protein comprising i) a DNA methyltransferase (DNMT) domain and/or a domain that recruits a DNMT (first recruiter domain), optionally wherein the DNMT domain and/or the first recruiter domain comprise a DNMT3A domain and/or a DNMT3L domain, and optionally wherein the DNMT recruited by the recruiter domain is DNMT3A; ii) a transcriptional repressor domain; iii) a DNA-binding domain comprising a dead CRISPR Cas (dCas) domain; or one or more nucleic acid molecules encoding the fusion protein; b) two or more gRNAs that each bind to a target region in a human gene selected from B2M, CIITA, TRAC, CD247, RFX5, RFXAP, CD3D, and CD3G, or a cis regulatory element regulating one or more of B2M, CIITA, TRAC, CD247, RFX5, RFXAP, CD3D, and CD3G, wherein the system does not generate a DNA break in the B2M, CIITA, TRAC, CD247, RFX5, RFXAP, CD3D, or CD3G gene. In some embodiments, the system further comprises a third gRNA. In some embodiments, the system represses transcription of B2M, RFXAP, and CD3D/G.

In some embodiments, the present disclosure provides a human cell comprising a system of this disclosure, or progeny of the cell. In some embodiments, the cell is a T lymphocyte or a NK cell. In some embodiments, the human cell is optionally a T lymphocyte or a NK cell, and optionally the cell was modified ex vivo. In some embodiments, the cell further comprises a chimeric antigen receptor (CAR) directed against at least one antigen.

In some aspects, the present disclosure provides a pharmaceutical composition comprising a system of this disclosure and a pharmaceutically acceptable excipient. Optionally the composition comprises lipid nanoparticles (LNPs) comprising the system, and/or the DNA-binding domain is a dCas domain and the LNPs further comprise one or more gRNAs. In some embodiments, the pharmaceutical composition further comprises a pharmaceutically acceptable excipient.

In some aspects, the present disclosure provides a method of treating a patient in need thereof, comprising administering a system of this disclosure, human cells of this disclosure, or a pharmaceutical composition of this disclosure to the patient. In some embodiments, the patient has cancer or autoimmune disease.

In some aspects, the present disclosure provides a system of this disclosure, human cells of this disclosure, or a pharmaceutical composition of this disclosure for use in treating a patient in need thereof, optionally in a method of this disclosure.

In some aspects, the present disclosure provides a use of a system of this disclosure or a human cell of this disclosure in the manufacture of a medicament for treating a patient in need thereof, optionally in a method of this disclosure.

In some aspects, the present disclosure provides an epigenetically modified human cell comprising a plurality of genomic loci, wherein the human cell is of a cell type that, in its naturally-occurring (wildtype) state, is characterized by expression of at least one gene product from each one of the plurality of genomic loci, wherein each one of the genomic loci comprises two alleles, and wherein both alleles of each one of the plurality of genomic loci comprise a naturally-occurring (wildtype) genomic sequence associated with expression of the at least one functional gene product, and wherein the epigenetically modified human cell is characterized by reduced or abolished expression (as compared to a wildtype cell of the same cell type) of two or more, or of all, of the plurality of genomic loci.

In some embodiments, the cell comprises an artificially hypermethylated genomic region in one allele, or in both alleles, of two or more, or of all, of the plurality of genomic loci. In some embodiments, the hypermethylated region is within 2 kb, 1 kb, or 500 bp of the transcription start site of two or more, or of all, of the plurality of genomic loci. In some embodiments, the human cell is an immune effector cell. In some embodiments, the human cell is a T lymphocyte. In some embodiments, the human cell is a CD4+ T lymphocyte. In some embodiments, the human cell comprises a chimeric antigen receptor (CAR).

In some aspects, the present disclosure provides a method, comprising administering the epigenetically modified human cell of this disclosure to a subject in need thereof.

In some aspects, the present disclosure provides a pharmaceutical formulation comprising a population of a plurality of an epigenetically modified human cell of this disclosure in a medium suitable for administration to a human subject. In some embodiments, the population comprises at least 100,000 cells, at least 200,000 cells, at least 500,000 cells, at least 1,000,000 cells, at least 2,000,000 cells, at least 5,000,000 cells, at least 10,000,000 cells, at least 20,000,000 cells, at least 50,000,000 cells, or at least 100,000,000 of the epigenetically modified human cells. In some embodiments, the population comprises at least 75%, at least 80%, at least 85%, at least 90%, at least 95%, at least 98%, at least 99%, or at least 99% of cells that do not express a functional gene product from two or more, or of all, of the plurality of genomic loci.

In some aspects, the present disclosure provides an epigenetically modified human cell of this disclosure, a method of this disclosure, or a pharmaceutical composition of this disclosure, wherein the epigenetically modified human cell does not express MHC class II and does not express MHC class I, optionally, wherein the epigenetically modified is an immune effector cell and wherein the epigenetically modified immune effector cell does not express CD3.

### BRIEF DESCRIPTION OF DRAWINGS

FIG. 1 shows a diagram of an exemplary method for measuring epigenetic editing in human cells.

FIG. 2 shows experimental results demonstrating that multiplex epigenetic silencing of three exemplary genes (B2M, CIITA, and CD247) in primary human T cells shows efficacy close to single-target epigenetic silencing at day 13, indicating that multiplex silencing results in treated cells that are characterized by significantly reduced levels of B2M, HLA-DR, and CD3 surface expression. Grey bars (left bar for each gene) show values measured for negative control treatment (epigenetic editor without guide RNA), normalized to a value of 100%, light blue bars (middle bar for each gene) shows percent of treated cells positive the respective target (as normalized to control) after targeting the respective target gene individually, and dark blue bars (right bar for each gene) shows percent of treated cells positive for the respective target (as normalized to control) after targeting all three target genes simultaneously.

FIGS. 3A-3D show the effect of simultaneous epigenetic silencing of B2M, CIITA, and CD247 in human T cells. CIITA silencing was assessed by measuring HLA-DR cell surface expression and CD247 silencing was assessed by measuring CD3 cell surface expression in the treated cells. FIG. 3A shows epigenetic silencing of target genes after nucleofection on Day 2, with lentiviral transduction carrying a BCMA CAR construct on Day 1, Day 2, or Day 3. FIG. 3B shows epigenetic silencing of target genes after nucleofection on Day 3, with lentiviral transduction carrying a BCMA CAR on Day 1, Day 2, or Day 3. FIGS. 3C-3D show epigenetic silencing of target genes after nucleofection on Day 3, with lentiviral transduction carrying a BCMA CAR (CAR+) or an empty vector (CAR−) on Day 1, Day 2, or Day 3, in chimeric antigen receptor (CAR) positive (FIG. 3C) and negative (FIG. 3D) cells from the same well.

FIG. 4 shows the transduction efficiency for Day 3 nucleofection shown by cell donor and day of transfection.

FIG. 5 shows multiplexed (here: duplexed) epigenetic silencing of B2M (left) and RFX5 (right) in human T cells over 22 days. Cells were restimulated at D14 with CD3/CD28 stimulation. RFX5 silencing was assessed by measuring cell surface expression of HLA-DR in the treated cell populations.

FIG. 6 shows flow cytometry data showing duplex epigenetic silencing of B2M and RFX5 in primary human T cells.

FIG. 7 shows multiplex silencing of B2M, CD3D/G, and RFX5 in primary human T cells by current methods (Cas9 KO and CRISPRi) and an exemplary epigenetic repressor fusion protein of this disclosure (CRISPR-Off) together with a combination of gRNAs targeting the three genes. RFX5 silencing was assessed by measuring cell surface expression of HLA-DR in the treated cell populations. Guides used included RNA138, RNA949, RNA2628, RNA2668, RNA2329, and RNA2510. Controls included wt Cas9 (positive control, Cas9 KO), epigenetic editor without guide (no guide, negative control), CRISPRi (dCas9, negative control for durability).

FIG. 8 shows silencing of B2M, RFX5, and CD3D/G in primary human T cells expressing a BCMA CAR over time. Guides used included RNA138, RNA949, RNA2628, RNA2668, RNA2329, and RNA2510. At Day 14 cells were restimulated with BCMA and RPMI 8226 cells to test durability of silencing post-CAR stimulation.

FIG. 9 shows that cells treated with exemplary epigenetic repressor fusion proteins (CRISPR-off) of this disclosure and multiplex targeting guide RNA combinations show significantly fewer adverse genetic events, such as indels (left) or translocations (right) compared to current Cas9-based, DNA-cutting gene editing methods. Events were identified using an unbiased, sequence-based methodology.

FIG. 10 shows a graph of translocation events in treated cells observed using fluorescence in situ hybridization (FISH).

FIG. 11 shows a graph of genomic rearrangement events in treated cells observed using fluorescence in situ hybridization (FISH).

FIG. 12 shows silencing of CD247, B2M and CIITA in multiplex epigenetically edited cells used to assess genomic integrity post-epigenetic editing.

FIG. 13 shows a non-limiting experimental workflow for assessing genomic integrity in epigenetically edited cells.

FIG. 14 shows a diagram of a non-limiting example of an unbiased, sequence-based methodology.

FIG. 15 is a diagram showing examples of genetic events that may be detected by unbiased, sequence-based methods, such as indels (middle) and translocations (right).

FIG. 16 shows the percent of B2M reads with indel events in cells treated with current CRISPR methods (top) or a non-limiting example of the compositions of this disclosure (bottom).

FIG. 17 shows the percent of B2M reads with translocation events in cells treated with current CRISPR methods (top) or a non-limiting example of the compositions of this disclosure (bottom).

FIG. 18 shows all indel and translocation events observed in wildtype Cas9-target sites after treatment of primary human T cells with either multiplexed wtCas9 gene editing systems targeting B2M, CD247, and CIITA; multiplexed epigenetic editor fusion proteins (CRISPR-off) targeting B2M, CD247, and CIITA; and in unedited human primary T cells. For each genotype observation, there are three pairs of bars. The first two bars show the occurrence of the genotype in an unedited cell; the second pair indicate the occurrence of the genotype in wild type Cas9-edited cells; and the third pair indicate the occurrence of the genotype in epigenetically edited cells. Within each pair, the left bar indicates day 3 results and the right bar indicates day 7 results. WT: naturally-occurring (wildtype) genomic sequence at the target site; indels: indel mutation at the target site; translocations are expressed by a pairing of genes, e.g., B2M_CD247 indicate a translocation between the B2M and CD247 target sites.

FIG. 19 shows all indel and translocation events observed in target sites used for multiplex epigenetic editing of B2M and CD247 after treatment of primary human T cells with either multiplexed wtCas9 gene editing systems targeting B2M, CD247, and CIITA; multiplexed epigenetic editor fusion proteins (CRISPR-off) targeting B2M, CD247, and CIITA; and in unedited human primary T cells. Only data for B2M and CD247 target sites are shown. For each genotype observation, there are three pairs of bars. The first two bars show the occurrence of the genotype in an unedited cell; the second pair indicate the occurrence of the genotype in wild type Cas9-edited cells; and the third pair indicate the occurrence of the genotype in epigenetically edited cells. Within each pair, the left bar indicates day 3 results and the right bar indicates day 7 results.

FIGS. 20A-20F show directional genomic hybridization images indicating genetic events in treated cells. FIG. 20A shows cells with no adverse events. FIG. 20B shows a reciprocal translocation between B2M (chr. 15) and CIITA (chr. 16) on day 7 in wildtype Cas9 treated cells. FIG. 20C shows chromosomes 15 and 16 in a cell with no translocations for comparison. FIG. 20D shows a reciprocal translocation between B2M and CIITA on day 3 in wildtype Cas9-treated cells. FIG. 20E shows an edit site truncation at CD247 on day 7 in wildtype Cas9-treated cells. FIG. 20F shows cells with additional abnormalities.

FIG. 21 shows a summary of genomic event occurrences observed.

FIG. 22 shows a summary of genomic events observed by type of event as measured by in-Site. Within each group of bars, the types of events from left to right are: gain, loss, truncation at edit sites, SCE/inv, and chromothripsis.

FIG. 23 shows a summary of genomic events observed by type of event as measured by SCREEN. Within each group of bars, the types of events from left to right are: translocations, insertion, inversion, loss, gain, and complex.

FIG. 24 shows a summary of translocation occurrences using SCREEN (top) and In-Site (bottom). Within each group of bars for the In-Site panel, the types of events from left to right are: complex translocations, other simple translocations, B2M/CIITA reciprocal translocations, CD247/CIITA reciprocal translocations, and CD247/B2M reciprocal translocations.

FIG. 25 shows a summary of dicentric, acentric, and complex events. Within each group of bars, the types of events from left to right are: chromothripsis (DAPI), complex, dicentric, and acentric.

FIG. 26 shows a translocation observed, at day 3 in a triple Cas9 KO sample, between Chr15q and Chr16p forming a dicentric event. The acentric partner is missing from the spread leading to monosomy of Chr 16.

FIG. 27 shows graphs reporting experimental results illustrating that multiplexed, epigenetic editing achieved a comparable or significantly improved, durable reduction in B2M, CD3, and HLA-DR as compared to both wtCas9 and CRISPRi through a PMA/ionomycin restimulation at Day 14.

FIGS. 28A-28B show graphs reporting experimental results demonstrating that triple silenced cells show comparable function to unedited CAR T. Experiments were performed as described in Example 3.

FIGS. 29A-29B show bar graphs reporting experimental results for CD107a expression in two different cell populations using NK cells from three different donors, each treated with five variants of HLA-E.

FIGS. 29C-29D show bar graphs reporting experimental results for NK degranulation, measured by cell surface CD107a expression in two different NK cell populations defined by NKG2C and NKG2A expression, in response to B2M KO T cells with or without expression of the indicated HLA-E variant.

FIG. 30A shows line graphs reporting the frequency of two different cell populations labeled with CFSE or CTV (B2M KO T cells and WT T cells or B2M KO T cells expressing the indicated HLA-E variant) among total viable dyed cells after co-incubation for 24 hours with NK cells at the indicated NK:T cell ratios. Mean+/−SEM. N=2 technical replicates from one donor. FIG. 30B shows a schematic (left) and line graphs (right) reporting the frequency of two different cell populations labeled with CFSE or CTV (B2M EE T cells and WT T cells or B2M EE T cells expressing the indicated HLA-E variant) among total viable dyed cells after co-incubation for 24 hours with NK cells at the indicated NK:T cell ratios. Mean+/−SEM. N=2 technical replicates from one donor. EE, epigenetic editor.

FIGS. 31A and 31B show line graphs the frequency of two different CFSE- or CTV-labeled K562 cell populations expressing different variants of HLA-E (as indicated by the last 3 residues of the variant peptide, i.e., “LIL” denotes VMAPRTLIL (SEQ ID NO: 674), “LVL” denotes VMAPRTLVL (SEQ ID NO: 676), and “VLL” denotes VMAPRTVLL (SEQ ID NO: 677)) among total viable dyed cells after co-incubation for 24 hours with NK cells at the indicated NK:T cell ratios. Mean+/−SEM. N=2 technical replicates from one donor.

FIG. 32 shows an experimental schematic and line graph results for a triple gene silencing experiment in primary T cells. Mean+/−SD. N=3 biological replicates from one donor.

FIG. 33A-34C shows a schematic (FIG. 33A, top) and results (FIG. 33A, bottom; FIG. 33B) for an allogeneic response graft-versus-host disease (GvHD) mixed lymphocyte reaction (MLR) assay using edited cells (as shown on the right). EE, epigenetic editor. EE Allo or Cas9 Allo indicate editing for B2M/RFXAP/CD3D/CD3G (in EE) and B2M/RFXAP/CD3D (in Cas9). Two biological replicates from 2 donors are summarized. Error bars represent +/−standard deviation. FIG. 33C shows the results of a similar experiment with the additional inclusion of HLA-E transduction.

FIG. 34A-34C shows a schematic (FIG. 34A, top) and results (FIG. 34A, bottom; FIG. 34B) for an alloresponse resistance mixed lymphocyte reaction (MLR) assay using edited cells (as shown on the right). EE, epigenetic editor. EE Allo or Cas9 Allo indicate editing for B2M/RFXAP/CD3D/CD3G (in EE) and B2M/RFXAP/CD3D (in Cas9) N=2 technical replicates from 1 donor. Error bars represent +/−standard deviation. Representative of results obtained with edited cells from 2 donors. FIG. 34C shows the results of a similar experiment with the additional inclusion of HLA-E transduction.

FIG. 35 show graphs reporting experimental results demonstrating that triple silenced cells show comparable function to unedited or Cas9-edited CAR T at three effector:target ratios. EE, epigenetic editor.

FIG. 36 is a graph reporting experimental results demonstrating that triple silenced cells have comparable anti-tumor function in vivo compared to unedited or Cas9-edited CAR T. EE, epigenetic editor. All mice in the PBS group were euthanized prior to the week 4 timepoint due to tumor burden, and all mice in the unedited CAR T group were euthanized prior to the week 5 timepoint due to GvHD.

FIG. 37 shows line graphs representing results from a multiplex gene silencing experiment in primary CAR T cells that had been previously edited and banked frozen. Day 0 is the day of thaw. Expression of B2M, HLA-DR, and CD3, is shown as measured by flow cytometry. T cells were restimulated by addition of tumor cells twice weekly. Mean+/−SD. N=2 technical replicates from each of two donors. Datapoints with less than 50% viability were excluded. EE, epigenetic editor.

FIG. 38 is a graph reporting experimental results demonstrating that triple silenced cells (lower dose) have comparable anti-tumor function in vivo compared to unedited or Cas9-edited CAR T. EE, epigenetic editor. All mice in the PBS group were euthanized prior to the week 4 timepoint due to tumor burden, and all mice in the unedited CAR T group were euthanized prior to the week 5 timepoint due to GvHD.

FIG. 39 shows T cell expansion in blood and proportion of hCD3+ cells in a multiplex epigenetic editing experiment.

FIG. 40 shows on-target methylation of both multiplex and single target CD3 conditions in 2 different donors.

### DETAILED DESCRIPTION

The present disclosure provides epigenetic editors for repressing expression of two or more human genes.

Epigenetic, multiplexed repression of genes in cells, e.g., in human cells, is broadly applicable in therapeutic applications, where repression of more than one gene is desired. Such therapeutic application include, without limitation allogeneic transplantation approaches, where donor and recipient are not perfectly matched. In such scenarios, MHC class I and MHC class II incompatibilities frequently result in rejection of the transplant by the recipient organism, and there is significant risk of graft-versus-host disease. In immunotherapeutic applications, expression of native CD3/T-cell receptor complexes on the surface of allogeneic immune effector cells is fraught with the risk of alloreactivity, which can lead to undesired, off-target toxicity. The repression of multiple genes in a cell or tissue can be effected by conventional gene editing methods, such as CRISPR-Cas gene editing. However, as such methods rely on cutting the target DNA, they are prone to undesired mutations, and where multiple loci are being targeted, to detrimental genomic translocations, which can pose a significant safety risk.

Some aspects of the present disclosure provide compositions, strategies, methods, and modalities for generating multiplexed epigenetically modified human cells characterized by repression of two or more genes without genomic alterations. Harnessing endogenous mechanisms of gene repression, including DNA methylation and histone modification, efficient, robust, and heritable silencing of two or more target genes can be effected, and the silenced state is heritable and persists over prolonged periods of time.

Some aspects of the present disclosure provide compositions, strategies, methods, and modalities for generating multiplexed epigenetically modified human immune effector cells, e.g., T cells, or CAR-T cells, characterized by repression of two or more genes associated with alloreactivity without genomic alterations. Adoptive cell transfer can involve either autologous or allogeneic cells. Thus far, most approaches approved for clinical use have utilized autologous cells. While allogeneic adoptive cell transfer has major advantages over autologous processes with regard to cost and scalability, the approach also comes with challenges: the use of allogeneic cells can cause severe graft-versus-host disease (GVHD) and host rejection or resistance, both of which will greatly impede any ultimate therapeutic benefit. Multiple cell types are involved in these problematic responses, including CD4+ T cells, CD8+ T cells, and natural killer (NK) cells. There are a variety of methods to avoid these responses when designing allogeneic immune cells, many targeting pathways known to cause alloreactivity. A number of such pathways have been identified, for example, MHC class I surface expression, MHC class II expression, and T-cell receptor (TCR) expression (also sometimes referred to as CD3 expression). Within each of these pathways, there are a variety of genetic targets—encoding subunits of protein complexes, for example—that are attractive for genetic engineering. β₂ macroglobulin (B2M) is known to contribute to MHC class I surface expression as well as CD8+ T cell response. Class II Major Histocompatibility Complex Transactivator (CIITA) and Regulatory Factor X5 (RFX5) contributes to MHC class II expression and CD4+ T cell alloresponse, while TCRα subunit constant (TRAC), CD247, CD3G and CD3D contribute to the T cell receptor complex and are therefore attractive targets in mitigating undesirable alloreactive outcomes.

Targeting more than one of these molecular entities through a genetic approach is beneficial: addressing the multiple pathways that cause phenomena problematic for allogeneic therapy simultaneously is an attractive proposition for a scalable, readily-manufactured cell-based product. With traditional gene editing, the risk of mutation, insertion, deletion, and chromothripsis increases with each targeted site. Thus, a multiplexed approach with improved safety (and, if needed, reversibility) is needed. Epigenetic editing can offer such a solution: with no changes to the underlying genome, the risk is mitigated and directing edits to multiple targets is more appropriate. An epigenetic editing approach can be used to manufacture cells with multiple alloreactive pathways reduced without risk of genome disruption, thus enabling a safe and readily-available product for patients.

By altering expression of target genes, the editors herein may be used to generate allogeneic cells (e.g., T cells, NK cells, etc.) with reduced alloreactivity. In some embodiments, altering expression of target genes provides a durable reduction in alloreactivity of allogeneic cells. In some embodiments, altering expression of two or more target genes provides a more durable reduction in alloreactivity of allogeneic cells than altering expression of one target gene. In some embodiments, altering expression of two or more target genes provides a greater reduction in alloreactivity of allogeneic cells than altering expression of one target gene.

### Target Genes

The present disclosure provides epigenetic editors for repressing expression of two or more target genes. In some embodiments, the target genes include one or more of B2M, CIITA, TRAC, CD247, RFX5, CD3D, and CD3G. In some embodiments, epigenetic editors target regions of a target gene. In some embodiments, a target region is a region near a transcriptional start site (TSS) of a target gene. In some embodiments, a target region is a cis-regulatory element (CRE) that modulates expression of a target gene. In some embodiments, a target region is a region within the coding sequence of a target gene.

### B2M

The present disclosure provides epigenetic editors for repressing expression of the human B2M gene. Unless otherwise stated, “B2M” (in italic) refers herein to a human B2M gene. A human B2M gene sequence can be found at Ensembl Accession No. ENSG00000166710. The present epigenetic editors have several advantages compared to other genome engineering methods, including reversibility, decreased risk of chromosomal translocation, and durable, inheritable silencing.

In some embodiments, the region of the human B2M gene targeted for epigenetic regulation is about 2 kb long, and is approximately +/−1 kb of the B2M TSS. In certain embodiments, the region has the nucleotide sequence of SEQ ID NO: 1284 (shown below). In some embodiments, the targeted B2M region is about 1 kb long, and is approximately +/−500 bps of the B2M TSS. In certain embodiments, the region targeted has the nucleotide sequence of SEQ ID NO: 1283 (shown below). The B2M TSS is at #chr15:55039548 of Genome GRCh38.

(SEQ ID NO: 1283)
TACCCAGAGAATGGAGAAACCCTGCAGGGAATTCCCAAGCTGTAGTTATAAACAGAAGTT
CTCCTTCTGCTAGGTAGCATTCAAAGATCTTAATCTTCTGGGTTTCCGTTTTCTCGAATG
AAAAATGCAGGTCCGAGCAGTTAACTGGCTGGGGCACCATTAGCAAGTCACTTAGCATCT
CTGGGGCCAGTCTGCAAAGCGAGGGGGCAGCCTTAATGTGCCTCCAGCCTGAAGTCCTAG
AATGAGCGCCCGGTGTCCCAAGCTGGGGCGCGCACCCCAGATCGGAGGGCGCCGATGTAC
AGACAGCAAACTCACCCAGTCTAGTGCATGCCTTCTTAAACATCACGAGACTCTAAGAAA
AGGAAACTGAAAACGGGAAAGTCCCTCTCTCTAACCTGGCACTGCGTCGCTGGCTTGGAG
ACAGGTGACGGTCCCTGCGGGCCTTGTCCTGATTGGCTGGGCACGCGTTTAATATAAGTG
GAGGCGTCGCGCTGGCGGGCATTCCTGAAGCTGACAGCATTCGGGCCGAGATGTCTCGCT
CCGTGGCCTTAGCTGTGCTCGCGCTACTCTCTCTTTCTGGCCTGGAGGCTATCCAGCGTG
AGTCTCTCCTACCCTCCCGCTCTGGTCCTTCCTCTCCCGCTCTGCACCCTCTGTGGCCCT
CGCTGTGCTCTCTCGCTCCGTGACTTCCCTTCTCCAAGTTCTCCTTGGTGGCCCGCCGTG
GGGCTAGTCCAGGGCTGGATCTCGGGGAAGCGGCGGGGTGGCCTGGGAGTGGGGAAGGGG
GTGCGCACCCGGGACGCGCGCTACTTGCCCCTTTCGGCGGGGAGCAGGGGAGACCTTTGG
CCTACGGCGACGGGAGGGTCGGGACAAAGTTTAGGGCGTCGATAAGCGTCAGAGCGCCGA
GGTTGGGGGAGGGTTTCTCTTCCGCTCTTTCGCGGGGCCTCTGGCTCCCCCAGCGCAGCT
GGAGTGGGGGACGGGTAGGCTCGTCCCAAAGGCGCGGCGCT
(SEQ ID NO: 1284)
GAGCCCTTTGTCTTCCAGTGTCTAAAATATTAATGTCAATGGAATCAGGCCAGAGTTTGA
ATTCTAGTCTCTTAGCCTTTGTTTCCCCTGTCCATAAAATGAATGGGGGTAATTCTTTCC
TCCTACAGTTTATTTATATATTCACTAATTCATTCATTCATCCATCCATTCGTTCATTCG
GTTTACTGAGTACCTACTATGTGCCAGCCCCTGTTCTAGGGTGGAAACTAAGAGAATGAT
GTACCTAGAGGGCGCTGGAAGCTCTAAAGCCCTAGCAGTTACTGCTTTTACTATTAGTGG
TCGTTTTTTTCTCCCCCCCGCCCCCCGACAAATCAACAGAACAAAGAAAATTACCTAAAC
AGCAAGGACATAGGGAGGAACTTCTTGGCACAGAACTTTCCAAACACTTTTTCCTGAAGG
GATACAAGAAGCAAGAAAGGTACTCTTTCACTAGGACCTTCTCTGAGCTGTCCTCAGGAT
GCTTTTGGGACTATTTTTCTTACCCAGAGAATGGAGAAACCCTGCAGGGAATTCCCAAGC
TGTAGTTATAAACAGAAGTTCTCCTTCTGCTAGGTAGCATTCAAAGATCTTAATCTTCTG
GGTTTCCGTTTTCTCGAATGAAAAATGCAGGTCCGAGCAGTTAACTGGCTGGGGCACCAT
TAGCAAGTCACTTAGCATCTCTGGGGCCAGTCTGCAAAGCGAGGGGGCAGCCTTAATGTG
CCTCCAGCCTGAAGTCCTAGAATGAGCGCCCGGTGTCCCAAGCTGGGGCGCGCACCCCAG
ATCGGAGGGCGCCGATGTACAGACAGCAAACTCACCCAGTCTAGTGCATGCCTTCTTAAA
CATCACGAGACTCTAAGAAAAGGAAACTGAAAACGGGAAAGTCCCTCTCTCTAACCTGGC
ACTGCGTCGCTGGCTTGGAGACAGGTGACGGTCCCTGCGGGCCTTGTCCTGATTGGCTGG
GCACGCGTTTAATATAAGTGGAGGCGTCGCGCTGGCGGGCATTCCTGAAGCTGACAGCAT
TCGGGCCGAGATGTCTCGCTCCGTGGCCTTAGCTGTGCTCGCGCTACTCTCTCTTTCTGG
CCTGGAGGCTATCCAGCGTGAGTCTCTCCTACCCTCCCGCTCTGGTCCTTCCTCTCCCGC
TCTGCACCCTCTGTGGCCCTCGCTGTGCTCTCTCGCTCCGTGACTTCCCTTCTCCAAGTT
CTCCTTGGTGGCCCGCCGTGGGGCTAGTCCAGGGCTGGATCTCGGGGAAGCGGCGGGGTG
GCCTGGGAGTGGGGAAGGGGGTGCGCACCCGGGACGCGCGCTACTTGCCCCTTTCGGCGG
GGAGCAGGGGAGACCTTTGGCCTACGGCGACGGGAGGGTCGGGACAAAGTTTAGGGCGTC
GATAAGCGTCAGAGCGCCGAGGTTGGGGGAGGGTTTCTCTTCCGCTCTTTCGCGGGGCCT
CTGGCTCCCCCAGCGCAGCTGGAGTGGGGGACGGGTAGGCTCGTCCCAAAGGCGCGGCGC
TGAGGTTTGTGAACGCGTGGAGGGGCGCTTGGGGTCTGGGGGAGGCGTCGCCCGGGTAAG
CCTGTCTGCTGCGGCTCTGCTTCCCTTAGACTGGAGAGCTGTGGACTTCGTCTAGGCGCC
CGCTAAGTTCGCATGTCCTAGCACCTCTGGGTCTATGTGGGGCCACACCGTGGGGAGGAA
ACAGCACGCGACGTTTGTAGAATGCTTGGCTGTGATACAAAGCGGTTTCGAATAATTAAC
TTATTTGTTCCCATCACATGTCACTTTTAAAAAATTATAAGAACTACCCGTTATTGACAT
CTTTCTGTGTGCCAAGGACTTTATGTGCTTTGCGTCATTTAATTTTGAAAACAGTTATCT
TCCGCCATAGATAACTACTATGGTTATCTTCTGCCTCTCACAGATGAAGAAACTAAGGCA
CCGAGATTTTAAGAAACTTAATTACACAGGGGATAAATGGCAGCAATCGAGATTGAAGTC
AAGCCTAACCAGGGCTTTTGC

### CIITA

The present disclosure provides epigenetic editors for repressing expression of the human CIITA gene. Unless otherwise stated, “CIITA” (in italic) refers herein to a human CIITA gene. A human CIITA gene sequence can be found at Ensembl Accession No. ENSG00000179583.21. The present epigenetic editors have several advantages compared to other genome engineering methods, including reversibility, decreased risk of chromosomal translocation, and durable, inheritable silencing. In some embodiments, CIITA silencing leads to decreased presence of the Human Leukocyte Antigen-DR isotype (HLA-DR) on a cell.

In some embodiments, the region of the human CIITA gene targeted for epigenetic regulation is about 2 kb long, and is approximately +/−1 kb of the CIITA TSS. In certain embodiments, the region has the nucleotide sequence of SEQ ID NO: 1314 (shown below). In some embodiments, the targeted CIITA region is about 1 kb long, and is approximately +/−500 bps of the CIITA TSS. In certain embodiments, the region targeted has the nucleotide sequence of SEQ ID NO: 1313 (shown below). TSS1 of CIITA is at #chr16:10877202 and TSS2 (alternative TSS) of CIITA is at #chr16:10878968 of Genome GRCh38: CM000678.2.

(SEQ ID NO: 1313)
TCCAATGTCTTGGGATGAAAATGACAGGTGGGCCACTTATGATCTCCAGAGAAATTCAGG
GCAATTTGGTGTGGGAGTAGGCATGGTAGAGGAGAGCAGCATCTAAGAAGTCCCCAGCAG
AGGCTCTCAGCTTGTCTTGAGGCATCTGGGCGGAGGGCTATGATACTGGCCCCATCCTGC
AGAAGGTGGCAGATATTGGCAGCTGGCACCAGTGCGGTTCCATTGTGATCATCATTTCTG
AACGTCAGACTGTTGAAGGTTCCCCCAACAGACTTTCTGTGCAACTTTCTGTCTTCACCA
AATTCAGTCCACAGTAAGGAAGTGAAATTAATTTCAGAGGTGTGGGGAGGGCTTAAGGGA
GTGTGGTAAAATTAGAGGGTGTTCAGAAACAGAAATCTGACCGCTTGGGGCCACCTTGCA
GGGAGAGTTTTTTTGATGATCCCTCACTTGTTTCTTTGCATGTTGGCTTAGCTTGGCGGG
CTCCCAACTGGTGACTGGTTAGTGATGAGGCTAGTGATGAGGCTGTGTGCTTCTGAGCTG
GGCATCCGAAGGCATCCTTGGGGAAGCTGAGGGCACGAGGAGGGGCTGCCAGACTCCGGG
AGCTGCTGCCTGGCTGGGATTCCTACACAATGCGTTGCCTGGCTCCACGCCCTGCTGGGT
CCTACCTGTCAGAGCCCCAAGGTAAAAAGGCCGGGAAAGCATCTTAATTTAGCGTGCAGT
CTCAGCTGGTCCTGCCATTCCAGATAAACAGAGAAACCATTCTGAATTGGGGATGGGGGT
GAGGATGGGAACAGGAGTCTGTGTCCTGCTGGGGCAGGCCATTGGAAGATGTGAAAGAGT
TGTCTATTTCCTTCCACCGGAGGGAGACTTCAGGTCAGCCAGGTGTCTGGAGTATGAACC
ATGTATCAGCACCGAAAGGTTCTAGAAGTCAGACTTTCGGGCAGTGTGTCACTAACTCTC
AGCATGCTGGCCTGGCTCGGCCCACAGCAAGGTCTTCTCGC
(SEQ ID NO: 1314)
ATAGGGTGTCACTATGTTGCCCAGGCTAGCCTCCAACTCCCGGCTTCAAGCAATCCTCCT
GCTTCGGCCTCCCAAAATGTTGGAATTACAGGCACAAGCCACCTGGCCCAGCCATCTACT
TTATATTCAAATAAAACTTTACGTCCCATTATAAAGGGAAAAAATGGCAAAAACAGGAGG
TAACCATTTAACAAGAAAGCAGAGTGATGTTAGATTATAGCAAGATACTGTTGACTGTAG
AAGGCTCTGAGGCTAGAGAGCTGCTTTCTATAAAACAGAGTGATCATATATTAGAAGAGG
TGTTAAAGACATGTTCACACCAAGCTGAGACTTCCTCCTTGATACCACCAGGAGGATGGG
CAGAGACTGGAAAAGACACTAACTTTCTCCCTATGGGAGTCAGTATTATTTAGCATCACT
TTGGCGGGTCACCCCAAACCATCTGACTACAAGGGTACCATATTTGGGTTAACACTCTTT
TGGTATAATTTATGTTTTAGTCCAATGTCTTGGGATGAAAATGACAGGTGGGCCACTTAT
GATCTCCAGAGAAATTCAGGGCAATTTGGTGTGGGAGTAGGCATGGTAGAGGAGAGCAGC
ATCTAAGAAGTCCCCAGCAGAGGCTCTCAGCTTGTCTTGAGGCATCTGGGCGGAGGGCTA
TGATACTGGCCCCATCCTGCAGAAGGTGGCAGATATTGGCAGCTGGCACCAGTGCGGTTC
CATTGTGATCATCATTTCTGAACGTCAGACTGTTGAAGGTTCCCCCAACAGACTTTCTGT
GCAACTTTCTGTCTTCACCAAATTCAGTCCACAGTAAGGAAGTGAAATTAATTTCAGAGG
TGTGGGGAGGGCTTAAGGGAGTGTGGTAAAATTAGAGGGTGTTCAGAAACAGAAATCTGA
CCGCTTGGGGCCACCTTGCAGGGAGAGTTTTTTTGATGATCCCTCACTTGTTTCTTTGCA
TGTTGGCTTAGCTTGGCGGGCTCCCAACTGGTGACTGGTTAGTGATGAGGCTAGTGATGA
GGCTGTGTGCTTCTGAGCTGGGCATCCGAAGGCATCCTTGGGGAAGCTGAGGGCACGAGG
AGGGGCTGCCAGACTCCGGGAGCTGCTGCCTGGCTGGGATTCCTACACAATGCGTTGCCT
GGCTCCACGCCCTGCTGGGTCCTACCTGTCAGAGCCCCAAGGTAAAAAGGCCGGGAAAGC
ATCTTAATTTAGCGTGCAGTCTCAGCTGGTCCTGCCATTCCAGATAAACAGAGAAACCAT
TCTGAATTGGGGATGGGGGTGAGGATGGGAACAGGAGTCTGTGTCCTGCTGGGGCAGGCC
ATTGGAAGATGTGAAAGAGTTGTCTATTTCCTTCCACCGGAGGGAGACTTCAGGTCAGCC
AGGTGTCTGGAGTATGAACCATGTATCAGCACCGAAAGGTTCTAGAAGTCAGACTTTCGG
GCAGTGTGTCACTAACTCTCAGCATGCTGGCCTGGCTCGGCCCACAGCAAGGTCTTCTCG
CCTCCCTTTGGGTAAATACTGAGGGGTGCCTCTGCAGGACGGGACCTCTGCCAGACTCCA
CTCCATACCCAGAGAAGCAGGGAAACCAAAATTGGAGTCAGCCTTGAGGTGTAGCTGTTG
AGCCCTCAGCAGCTGGGGAGAGCTGGCGGATGCTGCCCTCCCCCCAGTTTCCTAATGGTG
TTGTTTAAAAAGGGTCAGGGGACGGGGGAACAGATGGTGGGAAGAGCACAGTGCAGACAC
CTGGCACCGGCTCTGAAGGCAGCATGGCAGCTACACCGTTGGCTGGGAAGGGTGTGCCCC
TGAAGAAGTCGTTTACATTCTCGAGTCAATTTTCCTGGAGTGTACAATGGACCTGTGGGA
AAGCCTGTATGAAAGGGTAATGATGAGGGACCTAGCACAGTGTCCAATATTTTATAGGAA
CTGGAATTGAGCTCATAGGAGCTCAATTTTATTGGCATTGCTGTTGTTGGATGGTTAAAG
GGGTGGTATCCCTTTTCTCAG

### TRAC

The present disclosure provides epigenetic editors for repressing expression of the human TRAC gene. Unless otherwise stated, “TRAC” (in italic) refers herein to a human TRAC gene. A human TRAC gene sequence can be found at Ensembl Accession No. ENSG00000277734. The present epigenetic editors have several advantages compared to other genome engineering methods, including reversibility, decreased risk of chromosomal translocation, and durable, inheritable silencing.

In some embodiments, the region of the human TRAC gene targeted for epigenetic regulation is about 2 kb long, and is approximately +/−1 kb of the TRAC TSS. In certain embodiments, the region has the nucleotide sequence of SEQ ID NO: 1220 (shown below). In some embodiments, the targeted TRAC region is about 1 kb long, and is approximately +/−500 bps of the TRAC TSS. In certain embodiments, the region targeted has the nucleotide sequence of SEQ ID NO: 1219 (shown below). The TRAC TSS is at #chr14:22547506 of Genome GRCh38: CM000676.2.

(SEQ ID NO: 1219)
TGTGATAGATTTCCCAACTTAATGCCAACATACCATAAACCTCCCATTCTGCTAATGCCC
AGCCTAAGTTGGGGAGACCACTCCAGATTCCAAGATGTACAGTTTGCTTTGCTGGGCCTT
TTTCCCATGCCTGCCTTTACTCTGCCAGAGTTATATTGCTGGGGTTTTGAAGAAGATCCT
ATTAAATAAAAGAATAAGCAGTATTATTAAGTAGCCCTGCATTTCAGGTTTCCTTGAGTG
GCAGGCCAGGCCTGGCCGTGAACGTTCACTGAAATCATGGCCTCTTGGCCAAGATTGATA
GCTTGTGCCTGTCCCTGAGTCCCAGTCCATCACGAGCAGCTGGTTTCTAAGATGCTATTT
CCCGTATAAAGCATGAGACCGTGACTTGCCAGCCCCACAGAGCCCCGCCCTTGTCCATCA
CTGGCATCTGGACTCCAGCCTGGGTTGGGGCAAAGAGGGAAATGAGATCATGTCCTAACC
CTGATCCTCTTGTCCCACAGATATCCAGAACCCTGACCCTGCCGTGTACCAGCTGAGAGA
CTCTAAATCCAGTGACAAGTCTGTCTGCCTATTCACCGATTTTGATTCTCAAACAAATGT
GTCACAAAGTAAGGATTCTGATGTGTATATCACAGACAAAACTGTGCTAGACATGAGGTC
TATGGACTTCAAGAGCAACAGTGCTGTGGCCTGGAGCAACAAATCTGACTTTGCATGTGC
AAACGCCTTCAACAACAGCATTATTCCAGAAGACACCTTCTTCCCCAGCCCAGGTAAGGG
CAGCTTTGGTGCCTTCGCAGGCTGTTTCCTTGCTTCAGGAATGGCCAGGTTCTGCCCAGA
GCTCTGGTCAATGATGTCTAAAACTCCTCTGATTGGTGGTCTCGGCCTTATCCATTGCCA
CCAAAACCCTCTTTTTACTAAGAAACAGTGAGCCTTGTTCTGGCAGTCCAGAGAATGACA
CGGGAAAAAAGCAGATGAAGAGAAGGTGGCAGGAGAGGGCA
(SEQ ID NO: 1220)
CATGCTAATCCTCCGGCAAACCTCTGTTTCCTCCTCAAAAGGCAGGAGGTCGGAAAGAAT
AAACAATGAGAGTCACATTAAAAACACAAAATCCTACGGAAATACTGAAGAATGAGTCTC
AGCACTAAGGAAAAGCCTCCAGCAGCTCCTGCTTTCTGAGGGTGAAGGATAGACGCTGTG
GCTCTGCATGACTCACTAGCACTCTATCACGGCCATATTCTGGCAGGGTCAGTGGCTCCA
ACTAACATTTGTTTGGTACTTTACAGTTTATTAAATAGATGTTTATATGGAGAAGCTCTC
ATTTCTTTCTCAGAAGAGCCTGGCTAGGAAGGTGGATGAGGCACCATATTCATTTTGCAG
GTGAAATTCCTGAGATGTAAGGAGCTGCTGTGACTTGCTCAAGGCCTTATATCGAGTAAA
CGGTAGTGCTGGGGCTTAGACGCAGGTGTTCTGATTTATAGTTCAAAACCTCTATCAATG
AGAGAGCAATCTCCTGGTAATGTGATAGATTTCCCAACTTAATGCCAACATACCATAAAC
CTCCCATTCTGCTAATGCCCAGCCTAAGTTGGGGAGACCACTCCAGATTCCAAGATGTAC
AGTTTGCTTTGCTGGGCCTTTTTCCCATGCCTGCCTTTACTCTGCCAGAGTTATATTGCT
GGGGTTTTGAAGAAGATCCTATTAAATAAAAGAATAAGCAGTATTATTAAGTAGCCCTGC
ATTTCAGGTTTCCTTGAGTGGCAGGCCAGGCCTGGCCGTGAACGTTCACTGAAATCATGG
CCTCTTGGCCAAGATTGATAGCTTGTGCCTGTCCCTGAGTCCCAGTCCATCACGAGCAGC
TGGTTTCTAAGATGCTATTTCCCGTATAAAGCATGAGACCGTGACTTGCCAGCCCCACAG
AGCCCCGCCCTTGTCCATCACTGGCATCTGGACTCCAGCCTGGGTTGGGGCAAAGAGGGA
AATGAGATCATGTCCTAACCCTGATCCTCTTGTCCCACAGATATCCAGAACCCTGACCCT
GCCGTGTACCAGCTGAGAGACTCTAAATCCAGTGACAAGTCTGTCTGCCTATTCACCGAT
TTTGATTCTCAAACAAATGTGTCACAAAGTAAGGATTCTGATGTGTATATCACAGACAAA
ACTGTGCTAGACATGAGGTCTATGGACTTCAAGAGCAACAGTGCTGTGGCCTGGAGCAAC
AAATCTGACTTTGCATGTGCAAACGCCTTCAACAACAGCATTATTCCAGAAGACACCTTC
TTCCCCAGCCCAGGTAAGGGCAGCTTTGGTGCCTTCGCAGGCTGTTTCCTTGCTTCAGGA
ATGGCCAGGTTCTGCCCAGAGCTCTGGTCAATGATGTCTAAAACTCCTCTGATTGGTGGT
CTCGGCCTTATCCATTGCCACCAAAACCCTCTTTTTACTAAGAAACAGTGAGCCTTGTTC
TGGCAGTCCAGAGAATGACACGGGAAAAAAGCAGATGAAGAGAAGGTGGCAGGAGAGGGC
ACGTGGCCCAGCCTCAGTCTCTCCAACTGAGTTCCTGCCTGCCTGCCTTTGCTCAGACTG
TTTGCCCCTTACTGCTCTTCTAGGCCTCATTCTAAGCCCCTTCTCCAAGTTGCCTCTCCT
TATTTCTCCCTGTCTGCCAAAAAATCTTTCCCAGCTCACTAAGTCAGTCTCACGCAGTCA
CTCATTAACCCACCAATCACTGATTGTGCCGGCACATGAATGCACCAGGTGTTGAAGTGG
AGGAATTAAAAAGTCAGATGAGGGGTGTGCCCAGAGGAAGCACCATTCTAGTTGGGGGAG
CCCATCTGTCAGCTGGGAAAAGTCCAAATAACTTCAGATTGGAATGTGTTTTAACTCAGG
GTTGAGAAAACAGCTACCTTCAGGACAAAAGTCAGGGAAGGGCTCTCTGAAGAAATGCTA
CTTGAAGATACCAGCCCTACCAAGGGCAGGGAGAGGACCCTATAGAGGCCTGGGACAGGA
GCTCAATGAGAAAGGAGAAGA

### CD247

The present disclosure provides epigenetic editors for repressing expression of the human CD247 gene, which encodes the T-cell receptor zeta chain (also known as T3Z, CD3H, CD3Q, CD3Z, TCRZ, IMD25, CD3ZETA, and CD3-ZETA). Unless otherwise stated, “CD247” (in italic) refers herein to a human CD247 gene. A human CD247 gene sequence can be found at Ensembl Accession No. ENSG00000198821. The present epigenetic editors have several advantages compared to other genome engineering methods, including reversibility, decreased risk of chromosomal translocation, and durable, inheritable silencing. In some embodiments, silencing of CD247 leads to decreased presence of CD3 on a cell.

In some embodiments, the region of the human CD247 gene targeted for epigenetic regulation is about 2 kb long, and is approximately +/−1 kb of the CD247 transcription start site (TSS). In some embodiments, the targeted CD247 region is about 1,000 bps long, and is approximately +/−500 bps of the CD247 TSS.

### RFX5

The present disclosure provides epigenetic editors for repressing expression of the human RFX5 gene. Unless otherwise stated, “RFX5” (in italic) refers herein to a human RFX5 gene. A human RFX5 gene sequence can be found at Ensembl Accession No. ENSG00000143390. The present epigenetic editors have several advantages compared to other genome engineering methods, including reversibility, decreased risk of chromosomal translocation, and durable, inheritable silencing.

In some embodiments, the region of the human RFX5 gene targeted for epigenetic regulation is about 2 kb long, and is approximately +/−1 kb of the RFX5 transcription start site (TSS). In some embodiments, the targeted RFX5 region is about 1 kb long, and is approximately +/−500 bps of the RFX5 TSS. In certain embodiments, the region targeted has the nucleotide sequence of SEQ ID NO: 1286 (shown below). The RFX5 TSS is at chromosome 1:151347245 of Genome GRCh38. Alternative transcripts of RFX5, using a different TSS, are known in the human genome. One exemplary alternative transcript uses a TSSs located at Chromosome 1:151,347,326 of Genome GRCh38. In some embodiments, the targeted RFX5 region is about 1 kb long, and is approximately +/−500 bps of two or more, or all, RFX5 TSSs provided herein.

(SEQ ID NO: 1288)
TACTCCTCACCATTGCATATGCCTGGGAGGTCAGAAAGGATACGAACATATTCTGCCCTTCTAGGCTT
GGaaaaaaaaCTGGGCCCAAGTTCTCATTACTTAATCTCGCCACGACTTCCCCCGCTGCTCCCCCTCC
CCACGTCGCCGGCCCCTACGTCATCTCCCACAACAGCCCATCCTGAGGCGCGCCCCCACCTCCTTCCC
TTCCCCCTCCATTCAATCCCCGGTTTCTGCAAAGTGGAGCGGGGGTGGGGCGGGGTAGCGGAACTCAT
GCGCTGGGCGTCAGAAACCCGGCAGGGGAATGGGGTCCCAGATTCCGGGCACGTGCACATGGGTGAAG
AAGCTGTTGAAAATACAGCTACAATCTTTTTCTGCTCTCCTCCCAACTCATCTTGCCCCTCCCTACCT
CTGAACAGTCGCCTTCCAACACAAGCAACAATAAAGGTAAGGGAGAAAGAGAAACTACCTCGTAGCCA
CCTTCACTCCAGCCCAGCCCCAAGGTCCTGGCCCAGCCGGCTCGTGCGTTGCTTTATTTTCCTTTTTC
TGAATGGTTGTAACAGCCGCGATCTGCCTAGATACACGGGCGGCCTGCTCTGTCATTGGTCACTGCTG
CCACCCGGAGACAGAAGGCGCCGGGCTCGTACCGGCGGCCGCTGAAGTCTGCCTGGTAACAGAATCTG
ATTGGTCAATAGGGGAGGGAAAATCCAAAGTTGGTCTTATTTGAGTCAAGAGGGGGAAGTCCGGGTTG
GGTTTCGGTTAAAACTGGGGTCGGTTGGGACTGCAGGTCCCAAGAGGTTCTTCTCTTAAAGGGACCGC
AGGATGAAAGAACGGGCGCTCGGCTCCCTTTGGAACTGTCTTTTCTTTTGCCCTCCCCTTTTGTGTTT
CAGTTTAGTCTACGGTCCTCAGAGCTGCCGGAAGAGAAGAATCAATGGATGATAAAATAACTAGACAA
TGATGGGTAGGAGTGATGGAAGTTCACTGTAAGCTCCTATAACCCCCCA

### RFXAP

The present disclosure provides epigenetic editors for repressing expression of the human RFXAP gene. Unless otherwise stated, “RFXAP” refers herein to a human RFXAP (Regulatory Factor X Associated Protein) gene. The human RFXAP gene is well known to those of skill in the art. An exemplary human RFXAP gene sequence can be found at Ensembl Accession No. ENSG00000133111, and exemplary coordinates for the human RFXAP gene, based on the GRCh38 Genome Reference Consortium Human Build 38, also referred to herein as “hg38” are: Chromosome 13:36,819,222-36,829,104 (forward strand).

In some embodiments, the region of the human RFXAP gene targeted for epigenetic regulation is about 2 kb long, and is approximately +/−1 kb of the RFXAP transcription start site (TSS). In some embodiments, the targeted RFXAP region is about 1 kb long, and is approximately +/−500 bps of the RFXAP TSS. In certain embodiments, the region targeted has the nucleotide sequence of SEQ ID NO: 1283 (shown below). The RFXAP TSS is at chromosome 13:36819220-36819221 of Genome GRCh38.

Genomic sequence proximal to the RFXAP TSS (chr13: 36818720-36819720)—sequence shown comprises RFXAP genomic nucleotide sequence +/−500 bp from TSS:

(SEQ ID NO: 1498)
AGTTATCCCTCAATGCAAATACAACTCGGCCAAAGGTGTGGCCCATCCCTCTCCGCTCCACT
ATTTTCAAATAAGCCACCTCTGCCTTCATGGACTGCGAGCTAGTGACTCTAGGCTGTGACAC
CACTGCATCCCAGCTTCAAATCTCCCCTTGCAGGCAGTTTGTGGGGGAAATAATAAAATTAG
CATCCTAACACTACAAGTCGTGAGACCGCTCAGGAACAATCCTAATTTGCATACTGGAACTC
CTGTAGAAGATACTTTGAACCGCTTTAGATACAATAGGATCAGGTAAAACGGCGCTGCCTTG
CCAGCCACTGCAGGTTTTCACCTGACACAGCCCCCAGACCCTGTCTACGAATCCCCCAGCTG
GCCGAAGGGGCCGCGGCCGCACATGCGCAGTCAGTCCCGTTGGTTGTGCGCTCAGCCCCGCC
CCCAGAGGCGCCGCGCTCCCTGACAGCCCGCGGGCGTACACACGCTGACGCGCAGGCTGCGG
TCGCGCAGGCGCAGTCGGGGCGCCTTCCCGGTATAGGCGCCTTTTACCCCAGCGTGTCCTGA
GTCTTTGGTTCGCGAAGTGCCGTTAGGCCAAGCAGGTGCTAAAAGCCCGGGGTCGTGGACCC
CGGCCAGGTCTTAGCAGCATGGAGGCGCAGGGTGTAGCGGAGGGCGCGGGGCCGGGCGCCGC
CAGCGGCGTGCCCCACCCCGCGGCCCTAGCCCCGGCTGCGGCTCCCACCTTGGCGCCAGCCT
CGGTGGCGGCCGCGGCCTCTCAATTCACCCTGCTAGTGATGCAACCCTGTGCTGGGCAGGAC
GAGGCTGCGGCCCCCGGGGGCAGCGTTGGGGGGGGCAAGCCCGTTAGGTACCTGTGCGAAGG
GGCCGGGGATGGCGAAGAGGAGGCTGGGGAGGACGAGGCGGACCTGTTAGACACTTCGGACC
CTCCGGGGGGAGGCGAGAGCGCGGCTAGTTTGGAGGATCTAGAGGACGAGGAGACTCACtcg
gggggcgag

The RFXAP gene and encoded gene products as well as their structure and inclusion in the MHC class II enhanceosome have previously been described. See, e.g., Masternak et al., Promoter-specific functions of CIITA and the MHC class II enhanceosome in transcriptional activation. EMBO J. 2002 Mar. 15; 21(6):1379-88, incorporated herein by reference in its entirety.

### CD3D/G

The present disclosure provides epigenetic editors for repressing expression of the human CD3D gene. Unless otherwise stated, “CD3-delta” or “CD3D” (in italic) refers herein to a human CD3-delta gene. The human CD3-delta (CD3D) gene is well known to those of skill in the art. An exemplary human CD3D gene sequence can be found at Ensembl Accession No. ENSG00000167286 and exemplary coordinates for the human CD3D gene, based on the GRCh38 Genome Reference Consortium Human Build 38, also referred to herein as “hg38” are: Chromosome 11: 118,339,075-118,342,705 (reverse strand).

In some embodiments, the region of the human CD3D gene targeted for epigenetic regulation is about 2 kb long, and is approximately +/−1 kb of a CD3D transcription start site (TSS). In some embodiments, the targeted CD3D region is about 1 kb long, and is approximately +/−500 bps of the CD3D TSS. In certain embodiments, the region targeted has the nucleotide sequence of SEQ ID NO: 1285 (shown below). The CD3D TSS is located at Chromosome 11: 118,342,705 of Genome GRCh38. Alternative transcripts of CD3D, using a different TSS, are known in the human genome. For two exemplary alternative transcripts, the TSSs are located at Chromosome 11: 118,342,647, and at Chromosome 11:118,342,631, respectively, of Genome GRCh38. In some embodiments, the targeted CD3D region is about 1 kb long, and is approximately +/−500 bps of two or more, or all, CD3D TSSs provided herein.

(SEQ ID NO: 1285)
Ttgcctaggttggagggcagtggcatgatcacagctcactgaagccttgaaatcccgggctcaagtga
ccctcctgcctcagcctctcaaatagctgggactacagttgtgcaccaccatgcctggctaattcttt
tgttttttgtaaagatagagtctcatcatgttgctcagactggtctcaaactcctggcctcaaaggat
cctcccacttcggcctcccaaagctctgggattactggtgtgagccaccgtgcctggcAAGAAACACT
TTCAAGTGGGCCTCACTCCCATCAGTAATGTCCCTCTCAGGTCCCTTCCCCCACCCACCTGGAGTAGC
CTTACCTTGCGAGAGAAGGGTAGCCAGTACCAGGCCAGAGAGAAACGTGCTATGTTCCATCTCCCAGC
GGAACTCATCCAGTAGATAAAGCCAGGTCACCGAACTATCAGCCTGGGTGAGAGCTGCCCTCCCCTAG
CTGACTCACAGGTACCGGAAAGAGGAGAGTGGGGGAGGAACTAGAAGATGTCTGCTTCTCTGCTTTCT
GCCGTTGATGGTGGGAAATTTTCAGAGTGGGGGTGGGCTAGGGGTAGGGTAGGAAGGAAGCGGTGTAA
ATTGCTCTATCTCTGAGCAGGGAGCTGGCAGAGAATATGGAAAAGGTGGCCTGAACTTTAGCGTCCCT
ATTGACAATGCAACCACATTTACACACAACCTAAACACTGCCACATCTCGAAGCCCCTTGAGAGAAGC
CGTCGGCCCCATAGCGCAAGCCGTAGCAGCTAGATTTCTCATGGAGGCTGATCTTTCTCAGGACCCTT
CACTAGGCAGCCAGGGACACCAGATCTAgcagcttcttgtcagtgggaggttgggctttagagacccc
agccagagatttgaatcctgggtccaatactgcctacctgtggggcctgggccagccataaaattttt
cagagtcttattccATTAGTACCATTATTAGGATTCAAACAAGATATT

The present disclosure provides epigenetic editors for repressing expression of the human CD3G gene. Unless otherwise stated, “CD3-gamma” or “CD3G” (in italic) refers herein to a human CD3-gamma gene. The human CD3-gamma (CD3G) gene is well known to those of skill in the art. An exemplary human CD3G gene sequence can be found at Ensembl Accession No. ENSG00000160654 and exemplary coordinates for the human CD3G gene, based on the GRCh38 Genome Reference Consortium Human Build 38, also referred to herein as “hg38” are: Chromosome 11: 118,344,344-118,355,161 (forward strand).

In some embodiments, the region of the human CD3G gene targeted for epigenetic regulation is about 2 kb long, and is approximately +/−1 kb of the CD3G TSS. In some embodiments, the targeted CD3G region is about 1 kb long, and is approximately +/−500 bps of the CG3G TSS. In certain embodiments, the region targeted has the nucleotide sequence of SEQ ID NO: 1286 (shown below). The CD3G TSS is located at #chr11: 118,344,344 of Genome GRCh38. Alternative transcripts of CD3G are known in the human genome, e.g., at least one alternative transcript is known to use the same CD3G TSS as described above.

(SEQ ID NO: 1286)
agcgccactgcactccagcctgggcaacaaagtgagactctgtct
caaaaaagaaaaagaaaaaaaaagtgaaaaaaaTTCCTGAATGAA
GGCCTGGACTGAGGTGGCTTTCCATTTGGAGGTCCAGCCCCAAGC
ATCTGAGAGTCCCTCCTAAATTCATTACCTACAGCAACAACAACT
AGCAACAAGTAACAACTGGCTACGATCCTAACAACTAATGACAGG
GACATTTATCTCCCTCATGAAGAAACGGTCCCAGGACCATCTCCC
ACCCAGCATCCATTGCGGTTCCCTGTGCAAGATGAGTCTCTGAGT
GGGAATCCAGCACTCTCTCCCTCTTCTTCCCCACCACCTTCACCC
TCCTTAACGGAAAAACAAAAGGCATCTGCACCTGCAGCCCTGCTG
AGGCCCCTGCTGCTCACACTTGCAGCAGAGGGTGGAGGCTCTGGG
TTCTTGCCTTCTCTCAAAGGCCCCAGCCCCAACAGTGATGGGTGG
AGCCAGTCTAgctgctgcacaggctggctggctggctggctgcta
agggctgctCCACGCTTTTGCCGGAGGACAGAGACTGACATGGAA
CAGGGGAAGGGCCTGGCTGTCCTCATCCTGGCTATCATTCTTCTT
CAAGGTAAGGGCCTACTAGGGGTCTGGAAGCCTGGGGAAGGGCTC
AAGGGAAGAGCCCATCACTAGTGAGACAGGAATATTGGTATCCCT
AACCTTCAGCCTACCTCTGCTGTCACCTTAGAGTTCAAAGAAGGG
CAAAATGGAGGCTCTTAACTGTTCTCTGCTAGAGAGAAACAGTGT
CCCATGGAGGAGAAGGAATCCTTGTCTCTGAAAAATGCAAACAGA
GTACTTAAATGGCTGAAGAGAGGACCCTGTTACCGCCATCTTAGA
TTTGAATGCAGCCCAAAAGGGCATAGGCCAAGAAACTAAAAGGAA
AAAGTATATGTTCCCTACTTCAGAGCTGGGGGCTAGCAGTCGACC
TAGGAAATGT

The CD3D and CD3G genes and encoded gene products as well as their structure and inclusion in T cell receptor complexes have previously been described. See, e.g., Morath et al., alpha/beta and gamma/delta T cell receptors: Similar but different. J Leukoc Biol. 2020 June; 107(6):1045-1055, incorporated herein by reference in its entirety. In some embodiments, the targeted DNA region is or comprises a cis regulatory element (CRE) that regulates CD3D and CD3G. In some embodiments, the CRE is a region of DNA that regulates gene transcription. In an embodiment, the CRE does not encode a protein. In some embodiments, the CRE region targeted is located at chr11: 118343286-118343806 of genome GRCh38. In some embodiments, the CRE region targeted has the nucleotide sequence of SEQ ID NO: 1287 referred to herein as CD3D/G-CRE1. In some embodiments, the targeted site may be 10 to 50 bps (e.g., 10 to 40, 10 to 30, 10 to 20, 15 to 30, 15 to 25, or 15 to 20 bps) in length. In some embodiments, the targeted strand in the targeted region is the sense strand of the gene. In other embodiments, the targeted strand in the targeted region is the antisense strand of the gene.

(SEQ ID NO: 1287)
TGAGCCTCCCGGTGAGACAAATGGACCTTTTTCCCCTGTGGCCTA
CGAGGATCTGAAACTCTTCACGCTGCTGCAGTtagactgtcactt
acctggggacagagtcatgcctgtcttgctcactgctgtatcttg
tgcctggcacataacgggagctctgcacatttttgttgGCTCACT
GACTGACTGGCTGAGGGAGATAGGGGCCTGAGATCCTGGACATTC
AGTCCGGGCTCTGGCCCCTGAAAATGTGCTGGCCTGTCCTCGGAA
TTGTTCCACCTATTGCCTTCCAGGCGCCTCTTTCATGATCTCAAA
AGAATAGTGAAAccaggtgccgtgtctcacgcctgtaatcccaac
actttgggaggctgaggcaggtggatcacaaggtcaggagttcga
gaccagcctgaccaacaaggtgaaaccccgtctctactaaaaata
caaaaattagccgggcttgctggcacgcacctgtaatctcagcta
ctcaggaggctgaggcaggagaatcg

In some embodiments, the targeted site may be 10 to 50 bps (e.g., 10 to 40, 10 to 30, 10 to 20, 15 to 30, 15 to 25, or 15 to 20 bps) in length. In some embodiments, the targeted strand in the targeted region is the sense strand of the gene. In other embodiments, the targeted strand in the targeted region is the antisense strand of the gene.

In some embodiments, an epigenetic editor as described herein may comprise one or more fusion proteins, wherein each fusion protein comprises a DNA-binding domain linked to one or more effector domains for epigenetic modification. In certain embodiments, where the DNA-binding domain is a polynucleotide guided DNA-binding domain, the epigenetic editor may further comprise one or more guide polynucleotides. DNA-binding domains, effector domains, and guide polynucleotides of an epigenetic editor as described herein may be selected, e.g., from those described below, in any functional combination.

The epigenetic editors described herein may be expressed in a host cell transiently, or may be integrated in a genome of the host cell; such cells and their progeny are also contemplated by the present disclosure. Both transiently expressed and integrated epigenetic editors or components thereof can effect stable epigenetic modifications. For example, after introducing to a host cell an epigenetic editor described herein, the target gene in the host cell may be stably or permanently repressed or silenced. In some embodiments, expression of the target gene is reduced or silenced for at least 1 week, at least 2 weeks, at least 3 weeks, at least 4 weeks, at least 5 weeks, at least 6 weeks, at least 7 weeks, at least 2 months, at least 3 months, at least 4 months, at least 5 months, at least 6 months, at least 1 year, at least 2 years, or for the entire lifetime of the cell or the subject carrying the cell, as compared to the level of expression in the absence of the epigenetic editor. The epigenetic modification may be inherited by the progeny of the host cells into which the epigenetic editor was introduced.

### I. DNA-BINDING DOMAINS

An epigenetic editor described herein may comprise one or more DNA-binding domains that direct the effector domain(s) of the epigenetic editor to target sequences within or close to a target gene locus. In some embodiments, an epigenetic editor described herein may comprise one or more DNA-binding domains that direct the effector domain(s) of the epigenetic editor to target sequences within a cis-regulatory element (CRE) that regulates a target gene. DNA-binding domain as described herein may be, e.g., a polynucleotide guided DNA-binding domain, a zinc finger protein (ZFP) domain, a transcription activator like effector (TALE) domain, a meganuclease DNA-binding domain, and the like. Examples of DNA-binding domains can be found in U.S. Pat. No. 11,162,114, which is incorporated by refence herein in its entirety.

In some embodiments, a DNA-binding domain described herein is encoded by its native coding sequence. In other embodiments, the DNA-binding domain is encoded by a nucleotide sequence that has been codon-optimized for optimal expression in human cells.

### A. Polynucleotide Guided DNA-Binding Domains

In some embodiments, a DNA-binding domain herein may be a protein domain directed by a guide nucleic acid sequence (e.g., a guide RNA sequence) to a target site in the target gene locus. In certain embodiments, the protein domain may be derived from a CRISPR-associated nuclease, such as a Class I or II CRISPR-associated nuclease. In some embodiments, the protein domain may be derived from a Cas nuclease such as a Type II, Type IIA, Type IIB, Type IIC, Type V, or Type VI Cas nuclease. In certain embodiments, the protein domain may be derived from a Class II Cas nuclease selected from Cas1, Cas1B, Cas2, Cas3, Cas4, Cas5, Cas6, Cas7, Cas8, Cas9, Cas10, Cas14a, Cas14b, Cas14c, CasX, CasY, CasPhi, C2c4, C2c8, C2c9, C2c10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, and homologues and modified versions thereof. “Derived from” is used to mean that the protein domain comprises the full polypeptide sequence of the parent protein, or comprises a variant thereof (e.g., with amino acid residue deletions, insertions, and/or substitutions). The variant retains the desired function of the parent protein (e.g., the ability to form a complex with the guide nucleic acid sequence and the target DNA).

In some embodiments, the CRISPR-associated protein domain may be a Cas9 domain described herein. Cas9 may, for example, refer to a polypeptide with at least about 85%, 90%, 91%, 92%, 93%, 94%, 95%, 96%, 97%, 98%, 99%, or 100% sequence identity and/or sequence similarity to a wildtype Cas9 polypeptide described herein. In some embodiments, said wildtype polypeptide is Cas9 from Streptococcus pyogenes (NCBI Ref. No. NC_002737.2 (SEQ ID NO: 1)) and/or UniProt Ref. No. Q99ZW2 (SEQ ID NO: 2). In some embodiments, said wildtype polypeptide is Cas9 from Staphylococcus aureus (SEQ ID NO: 3). In some embodiments, the CRISPR-associated protein domain is a Cpf1 domain or protein, or a polypeptide with at least about 85%, 90%, 91%, 92%, 93%, 94%, 95%, 96%, 97%, 98%, 99%, or 100% sequence identity and/or sequence similarity to a wildtype Cpf1 polypeptide described herein (e.g., Cpf1 from Francisella novicida (UniProt Ref. No. U2UMQ6 or SEQ ID NO: 4). In certain embodiments, the CRISPR-associated protein domain may be a modified form of the wildtype protein comprising one or more amino acid residue changes such as a deletion, an insertion, or a substitution; a fusion or chimera; or any combination thereof.

Cas9 sequences and structures of variant Cas9 orthologs have been described for various organisms. Exemplary organisms from which a Cas9 domain herein can be derived include, but are not limited to, Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficile, Finegoldia magna, Natranaerobius thermophilus, Pelotomaculum thermopropionium, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, and Acaryochloris marina. Cas9 sequences also include those from the organisms and loci disclosed in Chylinski et al., RNA Biol. (2013) 10(5):726-37.

In some embodiments, the Cas9 domain is from Streptococcus pyogenes (spCas9). In some embodiments, the Cas9 domain is from Staphylococcus aureus (saCas9).

Other Cas domains are also contemplated for use in the epigenetic editors herein. These include, for example, those from CasX (Cas12E) (e.g., SEQ ID NO: 5), CasY (Cas12d) (e.g., SEQ ID NO: 6), Casφ (CasPhi) (e.g., SEQ ID NO: 7), Cas12f1 (Cas14a) (e.g., SEQ ID NO: 8), Cas12f2 (Cas14b) (e.g., SEQ ID NO: 9), Cas12f3 (Cas14c) (e.g., SEQ ID NO: 10), and C2c8 (e.g., SEQ ID NO: 11).

For epigenetic editing, the nuclease-derived protein domain (e.g., a Cas9 or Cpf1 domain) may have reduced or no nuclease activity through mutations such that the protein domain does not cleave DNA or has reduced DNA-cleaving activity while retaining the ability to complex with the guide nucleic acid sequence (e.g., guide RNA) and the target DNA. For example, the nuclease activity may be reduced by at least 30%, 35%, 40%, 45%, 50%, 55%, 60%, 65%, 70%, 75%, 80%, 85%, 90%, 95%, or 99% compared to the wildtype domain. In some embodiments, a CRISPR-associated protein domain described herein is catalytically inactive (“dead”). Examples of such domains include, for example, dCas9 (“dead” Cas9), dCpf1, ddCpf1, dCasPhi, ddCas12a, dLbCpf1, and dFnCpf1. A dCas9 protein domain, for example, may comprise one, two, or more mutations as compared to wildtype Cas9 that abrogate its nuclease activity. The DNA cleavage domain of Cas9 is known to include two subdomains: the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A (in RuvC1) and H840A (in HNH) completely inactivate the nuclease activity of SpCas9. SaCas9, similarly, may be inactivated by the mutations D10A and N580A. In some embodiments, the dCas9 comprises at least one mutation in the HNH subdomain and/or the RuvC1 subdomain that reduces or abrogates nuclease activity. In some embodiments, the dCas9 only comprises a RuvC1 subdomain, or only comprises an HNH subdomain. It is to be understood that any mutation that inactivates the RuvC1 and/or the HNH domain may be included in a dCas9 herein, e.g., insertion, deletion, or single or multiple amino acid substitution in the RuvC1 domain and/or the HNH domain.

In some embodiments, a dCas9 protein herein comprises a mutation at position(s) corresponding to position D10 (e.g., D10A), H840 (e.g., H840A), or both, of a wildtype SpCas9 sequence as numbered in the sequence provided at UniProt Accession No. Q99ZW2 (SEQ ID NO: 2). In particular embodiments, the dCas9 comprises the amino acid sequence of dSpCas9 (D10A and H840A) (SEQ ID NO: 12).

In some embodiments, a dCas9 protein as described herein comprises a mutation at position(s) corresponding to position D10 (e.g., D10A), N580 (e.g., N580A), or both, of a wildtype SaCas9 sequence (e.g., SEQ ID NO: 3). In particular embodiments, the dCas9 comprises the amino acid sequence of dSaCas9 (D10A and N580A) (SEQ ID NO: 13).

Additional suitable mutations that inactivate Cas9 will be apparent to those of skill in the art based on this disclosure and knowledge in the field and are within the scope of this disclosure. Such mutations may include, but are not limited to, D839A, N863A, and/or K603R in SpCas9. The present disclosure contemplates any mutations that reduce or abrogate the nuclease activity of any Cas9 described herein (e.g., mutations corresponding to any of the Cas9 mutations described herein).

A dCpf1 protein domain may comprise one, two, or more mutations as compared to wildtype Cpf1 that reduce or abrogate its nuclease activity. The Cpf1 protein has a RuvC-like endonuclease domain that is similar to the RuvC domain of Cas9, but does not have an HNH endonuclease domain, and the N-terminal of Cpf1 does not have the alpha-helical recognition lobe of Cas9. In some embodiments, the dCpf1 comprises one or more mutations corresponding to position D917A, E1006A, or D1255A as numbered in the sequence of the Francisella novicida Cpf1 protein (FnCpf1; SEQ ID NO: 4). In certain embodiments, the dCpf1 protein comprises mutations corresponding to D917A, E1006A, D1255A, D917A/E1006A, D917A/D1255A, E1006A/D1255A, or D917A/E1006A/D1255A, or corresponding mutation(s) in any of the Cpf1 amino acid sequences described herein. In some embodiments, the dCpf1 comprises a D917A mutation. In particular embodiments, the dCpf1 comprises the amino acid sequence of dFnCpf1 (SEQ ID NO: 14).

Further nuclease inactive CRISPR-associated protein domains contemplated herein include those from, for example, dNmeCas9 (e.g., SEQ ID NO: 15), dCjCas9 (e.g., SEQ ID NO: 16), dSt1Cas9 (e.g., SEQ ID NO: 17), dSt3Cas9 (e.g., SEQ ID NO: 18), dLbCpf1 (e.g., SEQ ID NO: 19), dAsCpf1 (e.g., SEQ ID NO: 20), denAsCpf1 (e.g., SEQ ID NO: 21), dHFAsCpf1 (e.g., SEQ ID NO: 22), dRVRAsCpf1 (e.g., SEQ ID NO: 23), dRRAsCpf1 (e.g., SEQ ID NO: 24), dCasX (e.g., SEQ ID NO: 25), and dCasPhi (e.g., SEQ ID NO: 26).

In some embodiments, a Cas9 domain described herein may be a high fidelity Cas9 domain, e.g., comprising one or more mutations that decrease electrostatic interactions between the Cas9 domain and the sugar-phosphate backbone of DNA to confer increased target binding specificity. In certain embodiments, the high fidelity Cas9 domain may be nuclease inactive as described herein.

A CRISPR-associated protein domain described herein may recognize a protospacer adjacent motif (PAM) sequence in a target gene. A “PAM” sequence is typically a 2 to 6 bp DNA sequence immediately following the sequence targeted by the CRISPR-associated protein domain. The PAM sequence is required for CRISPR protein binding and cleavage but is not part of the target sequence. The CRISPR-associated protein domain may either recognize a naturally occurring or canonical PAM sequence or may have altered PAM specificity. CRISPR-associated protein domains that bind to non-canonical PAM sequences have been described in the art. For example, Cas9 domains that bind non-canonical PAM sequences have been described in Kleinstiver et al., Nature (2015) 523 (7561): 481-5 and Kleinstiver et al., Nat Biotechnol. (2015) 33:1293-8. Such Cas9 domains may include, for example, those from “VRER” SpCas9, “EQR” SpCas9, “VQR” SpCas9, “SpG Cas9,” “SpRYCas9,” and “KKH” SaCas9. Nuclease inactive versions of these Cas9 domains are also contemplated, such as nuclease inactive VRER SpCas9 (e.g., SEQ ID NO: 27), nuclease inactive EQR SpCas9 (e.g., SEQ ID NO: 28), nuclease inactive VQR SpCas9 (e.g., SEQ ID NO: 29), nuclease inactive SpG Cas9 (e.g., SEQ ID NO: 30), nuclease inactive SpRY Cas9 (e.g., SEQ ID NO: 31), and nuclease inactive KKH SaCas9 (e.g., SEQ ID NO: 32). Another example is the Cas9 of Francisella novicida engineered to recognize 5′-YG-3′ (where “Y” is a pyrimidine).

Additional suitable CRISPR-associated proteins, orthologs, and variants, including nuclease inactive variants and sequences, will be apparent to those of skill in the art based on this disclosure.

Guide RNAs that can be used in conjunction with the CRISPR-associated protein domains herein are further described in Section II below.

### B. Zinc Finger Protein Domains

In some embodiments, the DNA-binding domain of an epigenetic editor described herein comprises a zinc finger protein (ZFP) domain (or “ZF domain” as used herein). ZFPs are proteins that have at least one zinc finger, and bind to DNA in a sequence-specific manner. A “zinc finger” (ZF) or “zinc finger motif” (ZF motif) refers to a polypeptide domain comprising a beta-beta-alpha (ββα)-protein fold stabilized by a zinc ion. A ZF binds from two to four base pairs of nucleotides, typically three or four base pairs (contiguous or noncontiguous). Each ZF typically comprises approximately 30 amino acids. ZFP domains may contain multiple ZFs that make tandem contacts with their target nucleic acid sequence. A tandem array of ZFs may be engineered to generate artificial ZFPs that bind desired nucleic acid targets. ZFPs may be rationally designed by using databases comprising triplet (or quadruplet) nucleotide sequences and individual ZF amino acid sequences, in which each triplet or quadruplet nucleotide sequence is associated with one or more amino acid sequences of ZFs that bind the particular triplet or quadruplet sequence. See, e.g., U.S. Pat. Nos. 6,453,242, 6,534,261, and 8,772,453.

ZFPs are widespread in eukaryotic cells, and may belong to, e.g., C2H2 class, CCHC class, PHD class, or RING class. An exemplary motif characterizing one class of these proteins (C2H2 class) is -Cys-(X)2-4-Cys-(X)12-His-(X)3-5-His- (SEQ ID NO: 657), where X is any independently chosen amino acid. In some embodiments, a ZFP domain herein may comprise a ZF array comprising sequential C2H2-ZFs each contacting three or more sequential nucleotides.

A ZFP domain of an epigenetic editor described herein may include 2, 3, 4, 5, 6, 7, 8, 9, 10, 11, 12, or more ZFs. The ZFP domain may include an array of two-finger or three-finger units, e.g., 3, 4, 5, 6, 7, 8, 9 or 10 or more units, wherein each unit binds a sub-site in the target sequence. In some embodiments, a ZFP domain comprising at least three ZFs recognizes a target DNA sequence of 9 or 10 nucleotides. In some embodiments, a ZFP domain comprising at least four ZFs recognizes a target DNA sequence of 12 to 14 nucleotides. In some embodiments, a ZFP domain comprising at least six ZFs recognizes a target DNA sequence of 18 to 21 nucleotides.

In some embodiments, ZFs in a ZFP domain described herein are connected via peptide linkers. The peptide linkers may be, e.g., 2, 3, 4, 5, 6, 7, 8, 9, 10, 11, 12, 13, 14, 15, 16, 17, 18, 19, 20, or more amino acids in length. In some embodiments, a linker comprises 5 or more amino acids. In some embodiments, a linker comprises 7-17 amino acids. The linker may be flexible or rigid.

In some embodiments a zinc finger array may have the sequence:

(SEQ ID NO: 650)
SRPGERPFQCRICMRNFSXXXXXXXHXXTHTGEKPFQCRICMRNFS
XXXXXXXHXXTH[linker]FQCRICMRNFSXXXXXXXHXXTHTGE
KPFQCRICMRNFSXXXXXXXHXXTH[linker]PFQCRICMRNFSX
XXXXXXHXXTHTGEKPFQCRICMRNFSXXXXXXXHXXTHLRGS,

or a sequence at least 70%, 75%, 80%, 85%, 90%, 95%, 96%, 97%, 98%, or 99% identical thereto, where “XXXXXXX” represents the amino acids of the ZF recognition helix, which confers DNA-binding specificity upon the zinc finger; each X may be independently chosen. In the above sequence, “XX” in italics may be TR, LR or LK, and “[linker]” represents a linker sequence. In some embodiments, the linker sequence is TGSQKP (SEQ ID NO: 651); this linker may be used when sub-sites targeted by the ZFs are adjacent. In some embodiments, the linker sequence is TGGGGSQKP (SEQ ID NO: 652); this linker may be used when there is a base between the sub-sites targeted by the zinc fingers. The two indicated linkers may be the same or different.

ZFP domains herein may contain arrays of two or more adjacent ZFs that are directly adjacent to one another (e.g., separated by a short (canonical) linker sequence), or are separated by longer, flexible or structured polypeptide sequences. In some embodiments, directly adjacent fingers bind to contiguous nucleic acid sequences, i.e., to adjacent trinucleotides/triplets. In some embodiments, adjacent fingers cross-bind between each other's respective target triplets, which may help to strengthen or enhance the recognition of the target sequence, leading to the binding of overlapping sequences. In some embodiments, distant ZFs within the ZFP domain may recognize (or bind to) non-contiguous nucleotide sequences.

Exemplary target sequences are shown in Tables 1-3 below.

TABLE 1
ZFP Target Sequences Within B2M

| ZF Target No. | B2M Target Site | SEQ ID NO |
|---|---|---|
| ZFTAR001 | GCATCTCTGGGGCCAGTC | 700 |
| ZFTAR002 | CTGGGGCCAGTCTGCAAAG | 701 |
| ZFTAR003 | GCAAAGCGAGGGGGCAGCC | 702 |
| ZFTAR004 | TCGGAGGGCGCCGATGTA | 703 |
| ZFTAR005 | GATGTTTAAGAAGGCATGC | 704 |
| ZFTAR006 | GTCTCGTGATGTTTAAGAA | 705 |
| ZFTAR007 | GGAAACTGAAAACGGGAAA | 706 |
| ZFTAR008 | GGTTAGAGAGAGGGACTTT | 707 |
| ZFTAR009 | GTGGAGGCGTCGCGCTGGC | 708 |
| ZFTAR010 | TAGGAGAGACTCACGCTGGA | 709 |
| ZFTAR011 | GAGGAAGGACCAGAGCGGGA | 710 |
| ZFTAR012 | GCGGGAGAGGAAGGACCAG | 711 |
| ZFTAR013 | GTCACGGAGCGAGAGAGCA | 712 |
| ZFTAR014 | TTGGAGAAGGGAAGTCACG | 713 |
| ZFTAR015 | GGGGAAGCGGCGGGGTGGC | 714 |
| ZFTAR016 | GGGTGCGCACCCGGGACG | 715 |
| ZFTAR017 | GCCGAAAGGGGCAAGTAGCG | 716 |
| ZFTAR018 | GTCGCCGTAGGCCAAAGG | 717 |
| ZFTAR019 | GGCGACGGGAGGGTCGGG | 718 |
| ZFTAR020 | GGCGACGGGAGGGTCGGGA | 719 |
| ZFTAR021 | TCAGAGCGCCGAGGTTGGG | 720 |
| ZFTAR022 | AGCGCCGAGGTTGGGGGA | 721 |
| ZFTAR023 | AGCGCCGAGGTTGGGGGAG | 722 |
| ZFTAR024 | GCTGGGGGAGCCAGAGGCC | 723 |
| ZFTAR025 | GCAGCTGGAGTGGGGGACG | 724 |
| ZFTAR026 | GCTGGAGTGGGGGACGGG | 725 |
| ZFTAR027 | GGAGTGGGGGACGGGTAGG | 726 |
| ZFTAR028 | GAGTGGGGGACGGGTAGG | 727 |
| ZFTAR029 | GAGTGGGGGACGGGTAGGC | 728 |
| ZFTAR030 | GTGGGGGACGGGTAGGCT | 729 |
| ZFTAR031 | GAGGTTTGTGAACGCGTGG | 730 |
| ZFTAR032 | TGTGAACGCGTGGAGGGGC | 731 |
| ZFTAR033 | GTGAACGCGTGGAGGGGC | 732 |
| ZFTAR034 | GTGAACGCGTGGAGGGGCG | 733 |
| ZFTAR035 | GTCGCCCGGGTAAGCCTGT | 734 |
| ZFTAR036 | TAAGCCTGTCTGCTGCGGCT | 735 |
| ZFTAR037 | GAACTTAGCGGGCGCCTAG | 736 |
| ZFTAR038 | GAGGTGCTAGGACATGCGAA | 737 |
| ZFTAR039 | AGTGACATGTGATGGGAAC | 738 |
| ZFTAR040 | GATTGAAGTCAAGCCTAA | 739 |
| ZFTAR041 | AGTCAAGCCTAACCAGGGC | 740 |

TABLE 2
ZFP Target Sequences Within CIITA

| ZF Target No. | CIITA Target Site | SEQ ID NO |
|---|---|---|
| ZFTAR001 | GAAAATGACAGGTGGGCC | 741 |
| ZFTAR002 | TTGGTGTGGGAGTAGGCA | 742 |
| ZFTAR003 | GGGTGTTCAGAAACAGAA | 743 |
| ZFTAR004 | GGGGAAGCTGAGGGCACG | 744 |
| ZFTAR005 | GGTCAGGGGACGGGGGAA | 745 |
| ZFTAR006 | GGCTGGGAAGGGTGTGCC | 746 |
| ZFTAR007 | GCCGGTGTGGTTGGGTGC | 747 |
| ZFTAR008 | TAAGTTGCTACGGGAAAG | 748 |
| ZFTAR009 | GTTGGAGGGGGAGAAGTC | 749 |
| ZFTAR010 | GAGGACCCAGCAGGAATC | 750 |
| ZFTAR011 | GACAGGTAGGACCCAGCA | 751 |
| ZFTAR012 | CTGGAATGGCAGGACCAG | 752 |
| ZFTAR013 | GGAGTGGAGTCTGGCAGA | 753 |
| ZFTAR014 | GCCAACGGTGTAGCTGCC | 754 |
| ZFTAR015 | GGAGTCTGAGAAAAGGGA | 755 |
| ZFTAR016 | GCATGAGACTTTGAGGTG | 756 |
| ZFTAR017 | GATACTGTTGACTGTAGAA | 757 |
| ZFTAR018 | GACTGTAGAAGGCTCTGAG | 758 |
| ZFTAR019 | GCAATTTGGTGTGGGAGTA | 759 |
| ZFTAR020 | GAAGTCCCCAGCAGAGGCT | 760 |
| ZFTAR021 | GAGGCATCTGGGCGGAGGG | 761 |
| ZFTAR022 | TGGGGAAGCTGAGGGCACG | 762 |
| ZFTAR023 | GCTGAGGGCACGAGGAGGG | 763 |
| ZFTAR024 | AGGGCACGAGGAGGGGCTG | 764 |
| ZFTAR025 | GGAGCTGCTGCCTGGCTGG | 765 |
| ZFTAR026 | TGGGGATGGGGGTGAGGAT | 766 |
| ZFTAR027 | TGGGGGTGAGGATGGGAAC | 767 |
| ZFTAR028 | GAGGATGGGAACAGGAGTC | 768 |
| ZFTAR029 | GCAGCTGGGGAGAGCTGGC | 769 |
| ZFTAR030 | GCTGGGGAGAGCTGGCGGA | 770 |
| ZFTAR031 | GGGGAGAGCTGGCGGATGC | 771 |
| ZFTAR032 | GACGGGGGAACAGATGGTG | 772 |
| ZFTAR033 | GAACAGATGGTGGGAAGAG | 773 |
| ZFTAR034 | GTTGGCTGGGAAGGGTGTG | 774 |
| ZFTAR035 | TAGGAACTGGAATTGAGCT | 775 |
| ZFTAR036 | GATGGTTAAAGGGGTGGTA | 776 |
| ZFTAR037 | GGTGTGGTTGGGTGCAGCC | 777 |
| ZFTAR038 | GCAGCCTTAAGTTGCTACG | 778 |
| ZFTAR039 | GCTACGGGAAAGTGTTGGA | 779 |
| ZFTAR040 | GGAGGGGGAGAAGTCAGAG | 780 |
| ZFTAR041 | GGGGGAGAAGTCAGAGGTA | 781 |
| ZFTAR042 | GGAGGACCCAGCAGGAATC | 782 |
| ZFTAR043 | GTGGTATCAAGGAGGAAGT | 783 |
| ZFTAR044 | GTAGTCAGATGGTTTGGGG | 784 |
| ZFTAR045 | GAGAGCCTCTGCTGGGGAC | 785 |
| ZFTAR046 | ACAGAAAGTCTGTTGGGGG | 786 |
| ZFTAR047 | GAATTTGGTGAAGACAGAA | 787 |
| ZFTAR048 | GTGGACTGAATTTGGTGAA | 788 |
| ZFTAR049 | GCAACGCATTGTGTAGGAA | 789 |
| ZFTAR050 | TAGGACCCAGCAGGGCGTG | 790 |
| ZFTAR051 | GACAGGTAGGACCCAGCAG | 791 |
| ZFTAR052 | TGGGTATGGAGTGGAGTCT | 792 |
| ZFTAR053 | GGGGAGGGCAGCATCCGCC | 793 |
| ZFTAR054 | GTTGACTGTAGAAGGCTCTG | 794 |
| ZFTAR055 | GAGGCTGTGTGCTTCTGAG | 795 |

TABLE 3
ZFP Target Sequences Within TRAC

| ZF Target No. | TRAC Target Site | SEQ ID NO |
|---|---|---|
| ZFTAR001 | AGAGCAGTAAGGGGCAAAC | 796 |
| ZFTAR002 | AGGGGCTTAGAATGAGGC | 797 |
| ZFTAR003 | AGGGGCTTAGAATGAGGCC | 798 |
| ZFTAR004 | GAACACCTGCGTCTAAGCC | 799 |
| ZFTAR005 | GAGGCCTGGGACAGGAGCT | 800 |
| ZFTAR006 | GAGGGTTTTGGTGGCAATG | 801 |
| ZFTAR007 | GATGTAAGGAGCTGCTGTG | 802 |
| ZFTAR008 | GCAAAGGCAGGCAGGCAGG | 803 |
| ZFTAR009 | GCAGCTGGTTTCTAAGAT | 804 |
| ZFTAR010 | GCAGGAGCTGCTGGAGGCT | 805 |
| ZFTAR011 | GGAAACAGCCTGCGAAGGC | 806 |
| ZFTAR012 | GGAGGAAACAGAGGTTTGC | 807 |
| ZFTAR013 | GGTGGCAGGAGAGGGCACG | 808 |
| ZFTAR014 | GGTGTTGAAGTGGAGGAA | 809 |
| ZFTAR015 | GTAAACGGTAGTGCTGGGG | 810 |
| ZFTAR016 | GTCTGAGCAAAGGCAGGC | 811 |
| ZFTAR017 | GGGGCTCTGTGGGGCTGGC | 812 |
| ZFTAR018 | TAGGAAGGTGGATGAGGC | 813 |
| ZFTAR019 | TAGGAAGGTGGATGAGGCA | 814 |
| ZFTAR020 | TCAGAAAGCAGGAGCTGCT | 815 |
| ZFTAR021 | TGCGTCTAAGCCCCAGCA | 816 |
| ZFTAR022 | TGGGCTGGGGAAGAAGGT | 817 |
| ZFTAR023 | TGGGCTGGGGAAGAAGGTG | 818 |
| ZFTAR024 | AAAGCAGGAGCTGCTGGA | 819 |
| ZFTAR025 | AAAGCAGGAGCTGCTGGAG | 820 |
| ZFTAR026 | AAGGTGGCAGGAGAGGGC | 821 |
| ZFTAR027 | AAGGTGGCAGGAGAGGGCA | 822 |
| ZFTAR028 | AATGAGGCCTAGAAGAGCA | 823 |
| ZFTAR029 | AGGGTTTTGGTGGCAATG | 824 |
| ZFTAR030 | ATTGTGCCGGCACATGAA | 825 |
| ZFTAR031 | GAAAAAAGCAGATGAAGAG | 826 |
| ZFTAR032 | GAAACAGAGGTTTGCCGGA | 827 |
| ZFTAR033 | GAAACAGCCTGCGAAGGC | 828 |
| ZFTAR034 | GAGGAAACAGAGGTTTGC | 829 |
| ZFTAR035 | GAGGAAACAGAGGTTTGCC | 830 |
| ZFTAR036 | GCCTAGAAGAGCAGTAAGG | 831 |
| ZFTAR037 | GCTGGGGAAGAAGGTGTC | 832 |
| ZFTAR038 | GGAGAAATAAGGAGAGGCA | 833 |
| ZFTAR039 | GGCGGGGCTCTGTGGGGC | 834 |
| ZFTAR040 | GGCGGGGCTCTGTGGGGCT | 835 |
| ZFTAR041 | GGCTGGGGAAGAAGGTGTC | 836 |
| ZFTAR042 | GGTTGGGGCAAAGAGGGAA | 837 |
| ZFTAR043 | GTAAGGGCAGCTTTGGTG | 838 |
| ZFTAR044 | GTGGCAGGAGAGGGCACG | 839 |
| ZFTAR045 | GTTGGGGCAAAGAGGGAA | 840 |
| ZFTAR046 | GTTGGGGCAAAGAGGGAAA | 841 |
| ZFTAR047 | TAAGGGGCAAACAGTCTGA | 842 |
| ZFTAR048 | TGGGTTAATGAGTGACTGC | 843 |
| ZFTAR049 | GAAGAGAAGGTGGCAGGA | 844 |
| ZFTAR050 | GAAGCAAGGAAACAGCCTGC | 845 |
| ZFTAR051 | GAAGGCGTTTGCACATGCA | 846 |
| ZFTAR052 | GACCAGAGCTCTGGGCAGA | 847 |
| ZFTAR053 | GACTTTGCATGTGCAAAC | 848 |
| ZFTAR054 | GAGATGTAAGGAGCTGCT | 849 |
| ZFTAR055 | GATTGTGCCGGCACATGAA | 850 |
| ZFTAR056 | GCTGTTGTTGAAGGCGTT | 851 |
| ZFTAR057 | GGAATCTGGAGTGGTCTCC | 852 |
| ZFTAR058 | GGAGAGACTGAGGCTGGG | 853 |
| ZFTAR059 | GTTGTTGAAGGCGTTTGC | 854 |
| ZFTAR060 | TCTGAGGGTGAAGGATAG | 855 |
| ZFTAR061 | TGAGGAGGAAACAGAGGTT | 856 |

In some embodiments, the ZFP domain of the present epigenetic editor binds to a target sequence selected from any one of SEQ ID NOs: 700-740. In some embodiments, the ZFP domain of the present epigenetic editor binds to a target sequence selected from any one of SEQ ID NOs: 741-795. In some embodiments, the ZFP domain of the present epigenetic editor binds to a target sequence selected from any one of SEQ ID NOs: 796-856. The ZF may comprise the ZF framework sequence of SEQ ID NO: 650, or any other ZF framework known in the art.

### C. TALEs

In some embodiments, the DNA-binding domain of an epigenetic editor described herein comprises a transcription activator-like effector (TALE) domain. The DNA-binding domain of a TALE comprises a highly conserved sequence of about 33-34 amino acids, with a repeat variable di-residue (RVD) at positions 12 and 13 that is central to the recognition of specific nucleotides. TALEs can be engineered to bind practically any desired DNA sequence. Methods for programming TALEs are known in the art. For example, such methods are described in Carroll et al., Genet Soc Amer. (2011) 188 (4): 773-82; Miller et al., Nat Biotechnol. (2007) 25 (7): 778-85; Christian et al., Genetics (2008) 186 (2): 757-61; Li et al., Nucl Acids Res. (2010) 39 (1): 359-72; and Moscou et al., Science (2009) 326 (5959): 1501.

### D. Other DNA-Binding Domains

Other DNA-binding domains are contemplated for the epigenetic editors described herein. In some embodiments, the DNA-binding domain comprises an argonaute protein domain, e.g., from Natronobacterium gregoryi (NgAgo). NgAgo is a ssDNA-guided endonuclease that is guided to its target site by 5′ phosphorylated ssDNA (gDNA), where it produces double-strand breaks. In contrast to Cas9, the NgAgo-gDNA system does not require a protospacer-adjacent motif (PAM). Thus, using a nuclease inactive NgAgo (dNgAgo) can greatly expand the bases that may be targeted. The characterization and use of NgAgo have been described, e.g., in Gao et al., Nat Biotechnol. (2016) 34 (7): 768-73; Swarts et al., Nature (2014) 507 (7491): 258-61; and Swarts et al., Nucl Acids Res. (2015) 43 (10): 5120-9.

In some embodiments, the DNA-binding domain comprises an inactivated nuclease, for example, an inactivated meganuclease. Additional non-limiting examples of DNA-binding domains include tetracycline-controlled repressor (tetR) DNA-binding domains, leucine zippers, helix-loop-helix (HLH) domains, helix-turn-helix domains, β-sheet motifs, steroid receptor motifs, bZIP domains homeodomains, and AT-hooks.

### II. GUIDE POLYNUCLEOTIDES

Epigenetic editors described herein that comprise a polynucleotide guided DNA-binding domain may also include a guide polynucleotide that is capable of forming a complex with the DNA-binding domain. The guide polynucleotide may comprise RNA, DNA, or a mixture of both. For example, where the polynucleotide guided DNA-binding domain is a CRISPR-associated protein domain, the guide polynucleotide may be a guide RNA (gRNA). A “guide RNA” or “gRNA” refers to a nucleic acid that is able to hybridize to a target sequence and direct binding of the CRISPR-Cas complex to the target sequence. Methods of using guide polynucleotide sequences with programmable DNA-binding proteins (e.g., CRISPR-associated protein domains) for site-specific DNA targeting (e.g., to modify a genome) are known in the art.

A guide polynucleotide sequence (e.g., a gRNA sequence) may comprise two parts: 1) a nucleotide sequence comprising a “targeting sequence” that is complementary to a target nucleic acid sequence (“target sequence”), e.g., to a nucleic acid sequence comprised in a genomic target site; and 2) a nucleotide sequence that binds a polynucleotide guided DNA-binding domain (e.g., a CRISPR-Cas protein domain). The nucleotide sequence in 1) may comprise a targeting sequence that is 100% complementary to a genomic nucleic acid sequence, e.g., a nucleic acid sequence comprised in a genomic target site, and thus may hybridize to the target nucleic acid sequence. The nucleotide sequence in 1) may be referred to as, e.g., a CRISPR RNA, or crRNA. The nucleotide sequence in 2) may be referred to as a scaffold sequence of a guide nucleic acid, e.g., a tracrRNA, or an activating region of a guide nucleic acid, and may comprise a stem-loop structure. Parts 1) and 2) as described above may be fused to form one single guide (e.g., a single guide RNA, or sgRNA), or may be on two separate nucleic acid molecules. In some embodiments, a guide polynucleotide comprises parts 1) and 2) connected by a linker. In some embodiments, a guide polynucleotide comprises parts 1) and 2) connected by a non-nucleic acid linker, for example, a peptide linker or a chemical linker.

Part 2 (the scaffold sequence) of a guide polynucleotide as described herein may be, for example, as described in Jinek et al., Science (2012) 337:816-21; U.S. Patent Publication 2016/0208288; or U.S. Patent Publication 2016/0200779. Variants of part 2) are also contemplated by the present disclosure. For example, the tetraloop and stem loop of a gRNA scaffold (tracrRNA) sequence may be modified to include RNA aptamers, which can be bound by specific protein domains. In some embodiments, such modified gRNAs can be used to facilitate the recruitment of repressive or activating domains fused to the protein-interacting RNA aptamers.

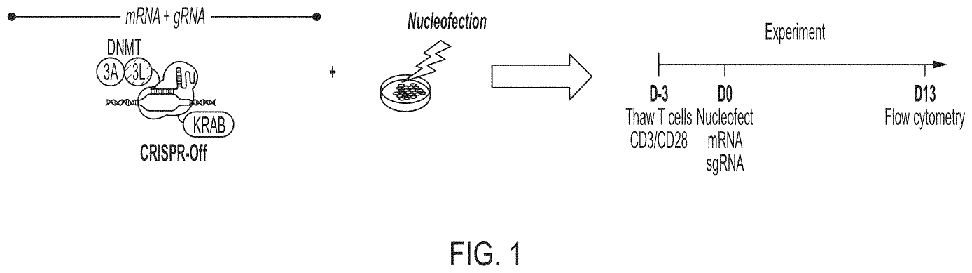

A gRNA as provided herein typically comprises a targeting domain and a binding domain. The targeting domain (also termed “targeting sequence”) may comprise a nucleic acid sequence that binds to a target site, e.g., to a genomic nucleic acid molecule within a cell. The target site may be a double-stranded DNA sequence comprising a PAM sequence as well as the target sequence, which is located on the same strand as, and directly adjacent to, the PAM sequence. The targeting domain of the gRNA may comprise an RNA sequence that corresponds to the target sequence, i.e., it resembles the sequence of the target domain, sometimes with one or more mismatches, but typically comprising an RNA sequence instead of a DNA sequence. The targeting domain of the gRNA thus may base pair (in full or partial complementarity) with the sequence of the double-stranded target site that is complementary to the target sequence, and thus with the strand complementary to the strand that comprises the PAM sequence. It will be understood that the targeting domain of the gRNA typically does not include a sequence that resembles the PAM sequence. It will further be understood that the location of the PAM may be 5′ or 3′ of the target sequence, depending on the nuclease employed. For example, the PAM is typically 3′ of the target sequence for Cas9 nucleases, and 5′ of the target sequence for Cas12a nucleases. For an illustration of the location of the PAM and the mechanism of gRNA binding to a target site, see, e.g., FIG. 1 of Vanegas et al., Fungal Biol Biotechnol. (2019) 6:6, which is incorporated by reference herein. For additional illustration and description of the mechanism of gRNA targeting of an RNA-guided nuclease to a target site, see Fu et al., Nat Biotechnol (2014) 32 (3): 279-84 and Sternberg et al., Nature (2014) 507 (7490): 62-7, each incorporated herein by reference.

In some embodiments, the targeting domain sequence comprises between 17 and 30 nucleotides and corresponds fully to the target sequence (i.e., without any mismatch nucleotides). In some embodiments, however, the targeting domain sequence may comprise one or more, but typically not more than 4, mismatches, e.g., 1, 2, 3, or 4 mismatches. As the targeting domain is part of gRNA, which is an RNA molecule, it will typically comprise ribonucleotides, while the DNA targeting domain will comprise deoxyribonucleotides.

An exemplary illustration of a Cas9 target site, comprising a 22 nucleotide target domain, and an NGG PAM sequence, as well as of a gRNA comprising a targeting domain that fully corresponds to the target sequence (and thus base pairs with full complementarity with the DNA strand complementary to the strand comprising the target sequence and PAM) is provided below:

[ target domain (DNA) ][ PAM ]
5′-N-N-N-N-N-N-N-N-N-N-N-N-N-N-N-N-N-N-N-N-N-N-N-G-G-3′ (DNA)
3′-N-N-N-N-N-N-N-N-N-N-N-N-N-N-N-N-N-N-N-N-N-N-N-C-C-5′ (DNA)
| | | | | | | | | | | | | | | | | | | | | |
5′-N-N-N-N-N-N-N-N-N-N-N-N-N-N-N-N-N-N-N-N-N-N-[ gRNA scaffold]-3′ (RNA)
[ target domain ( RNA) ][ binding domain ]

An exemplary illustration of a Cas12a target site, comprising a 22 nucleotide target domain, and a TTN PAM sequence, as well as of a gRNA comprising a targeting domain that fully corresponds to the target sequence (and thus base pairs with full complementarity with the DNA strand complementary to the strand comprising the target sequence and PAM) is provided below:

```
                    [ PAM ][ target domain ( DNA) ]
                    5′-T-T-N-N-N-N-N-N-N-N-N-N-N-N-N-N-N-N-N-N-N-N-N-N-N-3′ (DNA)
                    3′-A-A-N-N-N-N-N-N-N-N-N-N-N-N-N-N-N-N-N-N-N-N-N-N-N-5′ (DNA)
                                      | | | | | | | | | | | | | | | | | | | | | |
5′-[gRNA scaffold]-N-N-N-N-N-N-N-N-N-N-N-N-N-N-N-N-N-N-N-N-N-N-3′ (RNA)
[ binding domain ][ targeting domain ( RNA) ]
```

While not wishing to be bound by theory, at least in some embodiments, it is believed that the length and complementarity of the targeting domain with the target sequence contributes to specificity of the interaction of the gRNA/Cas9 molecule complex with a target nucleic acid. In some embodiments, the targeting domain of a gRNA provided herein is 5 to 50 nucleotides in length. In some embodiments, the targeting domain is 15 to 25 nucleotides in length. In some embodiments, the targeting domain is 18 to 22 nucleotides in length. In some embodiments, the targeting domain is 19-21 nucleotides in length. In some embodiments, the targeting domain is 15 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In certain embodiments, the targeting domain fully corresponds, without mismatch, to a target sequence provided herein, or a part thereof. In some embodiments, the targeting domain of a gRNA provided herein comprises 1 mismatch relative to a target sequence provided herein. In some embodiments, the targeting domain comprises 2 mismatches relative to the target sequence. In some embodiments, the target domain comprises 3 mismatches relative to the target sequence.

Methods for designing, selecting, and validating gRNAs are described herein and known in the art. Software tools can be used to optimize the gRNAs corresponding to a target DNA sequence, e.g., to minimize total off-target activity across the genome. For example, DNA sequence searching algorithms can be used to identify a target sequence in crRNAs of a gRNA for use with Cas9. Exemplary gRNA design tools include the ones described in Bae et al., Bioinformatics (2014) 30:1473-5.

Guide polynucleotides (e.g., gRNAs) described herein may be of various lengths. In some embodiments, the length of the spacer or targeting sequence depends on the CRISPR-associated protein component of the epigenetic editor system used. For example, Cas proteins from different bacterial species have varying optimal targeting sequence lengths. Accordingly, the spacer sequence may comprise, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more than 50 nucleotides in length. In some embodiments, the spacer comprises 10-24, 11-20, 11-16, 18-24, 19-21, or 20 nucleotides in length. In some embodiments, a guide polynucleotide (e.g., gRNA) is from 15-100 (e.g., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotides in length and comprises a spacer sequence of at least 10 (e.g., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) contiguous nucleotides complementary to the target sequence. In some embodiments, a guide polynucleotide described herein may be truncated, e.g., by 1, 2, 3, 4, 5, 10, 15, 20, 25, 30, 40, 50 or more nucleotides.

In certain embodiments, the 3′ end of the target gene target sequence is immediately adjacent to a PAM sequence (e.g., a canonical PAM sequence such as NGG for SpCas9). The degree of complementarity between the targeting sequence of the guide polynucleotide (e.g., the spacer sequence of a gRNA) and the target sequence may be at least 90%, 91%, 92%, 93%, 94%, 95%, 96%, 97%, 98%, 99%, or 100%. In particular embodiments, the targeting and the target sequence may be 100% complementary. In other embodiments, the targeting sequence and the target sequence may contain, e.g., 1, 2, 3, 4, 5, 6, 7, 8, 9, or 10 mismatches.

A guide polynucleotide (e.g., gRNA) may be modified with, for example, chemical alterations and synthetic modifications. A modified gRNA, for instance, can include an alteration or replacement of one or both of the non-linking phosphate oxygens and/or of one or more of the linking phosphate oxygens in the phosphodiester backbone linkage, an alteration of the ribose sugar (e.g., of the 2′ hydroxyl on the ribose sugar), an alteration of the phosphate moiety, modification or replacement of a naturally occurring nucleobase, modification or replacement of the ribose-phosphate backbone, modification of the 3′ end and/or 5′ end of the oligonucleotide, replacement of a terminal phosphate group or conjugation of a moiety, cap, or linker, or any combination thereof.

In some embodiments, one or more ribose groups of the gRNA may be modified. Examples of chemical modifications to the ribose group include, but are not limited to, 2′-O-methyl(2′-OMe), 2′-fluoro (2′-F), 2′-deoxy, 2′-O-(2-methoxyethyl) (2′-MOE), 2′-NH2, 2′-O-allyl, 2′-O-ethylamine, 2′-O-cyanoethyl, 2′-O-acetalester, or a bicyclic nucleotide such as locked nucleic acid (LNA), 2′-(5-constrained ethyl (S-cEt)), constrained MOE, or 2′-0,4′-C-aminomethylene bridged nucleic acid (2′,4′-BNANC). 2′-O-methyl modification and/or 2′-fluoro modification may increase binding affinity and/or nuclease stability of the gRNA oligonucleotides.

In some embodiments, one or more phosphate groups of the gRNA may be chemically modified. Examples of chemical modifications to a phosphate group include, but are not limited to, a phosphorothioate (PS), phosphonoacetate (PACE), thiophosphonoacetate (thioPACE), amide, triazole, phosphonate, and phosphotriester modification. In some embodiments, a guide polynucleotide described herein may comprise one, two, three, or more PS linkages at or near the 5′ end and/or the 3′ end; the PS linkages may be contiguous or noncontiguous.

In some embodiments, the gRNA herein comprises a mixture of ribonucleotides and deoxyribonucleotides and/or one or more PS linkages.

In some embodiments, one or more nucleobases of the gRNA may be chemically modified. Examples of chemically modified nucleobases include, but are not limited to, 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, and nucleobases with halogenated aromatic groups. Chemical modifications can be made in the spacer region, the tracr RNA region, the stem loop, or any combination thereof.

Tables 4-11 below list exemplary gRNA target sequences for epigenetic modification of human target genes, as well as the coordinates of the start positions of the targeted site on the relevant human chromosome (“SEQ” indicates SEQ ID NO). Some tables also show the distance from the start coordinate to the TSS coordinate of the target gene.

TABLE 4
Exemplary Target Sequences of gRNAs Targeting B2M

| | gRNA Targeting | SEQ | TSS | % | Start |
|---|---|---|---|---|---|
| gRNA | Sequence | ID | Distance | B2M | Nucleotide |
| number | (5′-3′) | NO | (bp) | pos. | on Chr15 |
| gRNA246 | CGGCUCUGCUUCCCUUAGAC | 679 | 570 | 29.6 | 44712088 |
| gRNA146 | UCUCCUUGGUGGCCCGCCGU | 680 | 198 | 30.2 | 44711716 |
| gRNA083 | CUCAUUCUAGGACUUCAGGC | 681 | −278 | 30.4 | 44711262 |
| gRNA119 | GGGCACGCGUUUAAUAUAAG | 682 | −43 | 31.1 | 44711475 |
| gRNA242 | CGCAGCAGACAGGCUUACCC | 683 | 549 | 31.6 | 44712089 |
| gRNA177 | CGCGCGCUACUUGCCCCUUU | 684 | 293 | 31.9 | 44711811 |
| gRNA154 | CCGUGGGGCUAGUCCAGGGC | 685 | 214 | 32.2 | 44711732 |
| gRNA247 | UCCCUUAGACUGGAGAGCUG | 686 | 580 | 32.8 | 44712098 |
| gRNA197 | GAGGGUCGGGACAAAGUUUA | 687 | 352 | 33.5 | 44711870 |
| gRNA249 | GUCCACAGCUCUCCAGUCUA | 688 | 582 | 34.1 | 44712122 |
| gRNA196 | GGAGGGUCGGGACAAAGUUU | 689 | 351 | 34.2 | 44711869 |
| gRNA105 | GCAGUGCCAGGUUAGAGAGA | 690 | −119 | 34.5 | 44711421 |
| gRNA271 | GGCCACGGAGCGAGACAUCU | 691 | 24 | 34.6 | 44711545 |
| gRNA245 | GAAGCAGAGCCGCAGCAGAC | 692 | 559 | 35.3 | 44712099 |
| gRNA248 | UCCACAGCUCUCCAGUCUAA | 693 | 581 | 35.4 | 44712121 |
| gRNA013 | UCCUGAAGCUGACAGCAUUC | 694 | 1 | 35.7 | 44711519 |
| gRNA223 | GACGGGUAGGCUCGUCCCAA | 695 | 468 | 36 | 44711986 |
| gRNA125 | AGGGUAGGAGAGACUCACGC | 696 | 91 | 36.4 | 44711631 |
| gRNA176 | GGGGCAAGUAGCGCGCGUCC | 697 | 287 | 36.4 | 44711827 |
| gRNA214 | UCCCCCAGCGCAGCUGGAGU | 698 | 444 | 36.7 | 44711962 |
| gRNA262 | CUAUGUGGGGCCACACCGUG | 699 | 651 | 36.7 | 44712169 |
| gRNA137 | GGAGCGAGAGAGCACAGCGA | 857 | 155 | 36.9 | 44711695 |
| gRNA189 | GACCUUUGGCCUACGGCGAC | 858 | 330 | 37.2 | 44711848 |
| gRNA139 | AACUUGGAGAAGGGAAGUCA | 859 | 176 | 37.4 | 44711716 |
| gRNA224 | GUAGGCUCGUCCCAAAGGCG | 860 | 473 | 37.6 | 44711991 |
| gRNA003 | GAAAGUCCCUCUCUCUAACC | 861 | −125 | 38 | 44711393 |
| gRNA015 | GAGUAGCGCGAGCACAGCUA | 862 | 45 | 38 | 44711585 |
| gRNA011 | AAGUGGAGGCGUCGCGCUGG | 863 | −26 | 38.1 | 44711492 |
| gRNA129 | GGGAGAGGAAGGACCAGAGC | 864 | 114 | 38.1 | 44711654 |
| gRNA140 | UCCCUUCUCCAAGUUCUCCU | 865 | 184 | 38.1 | 44711702 |
| gRNA162 | UGGAUCUCGGGGAAGCGGCG | 866 | 234 | 38.2 | 44711752 |
| gRNA006 | CGCGAGCACAGCUAAGGCCA | 867 | 39 | 38.5 | 44711579 |
| gRNA007 | ACUCUCUCUUUCUGGCCUGG | 868 | 64 | 38.8 | 44711582 |
| gRNA122 | GGCCGAGAUGUCUCGCUCCG | 869 | 22 | 38.8 | 44711540 |
| gRNA231 | AGGUUUGUGAACGCGUGGAG | 870 | 501 | 38.8 | 44712019 |
| gRNA016 | ACUCACGCUGGAUAGCCUCC | 871 | 79 | 39.2 | 44711619 |
| gRNA238 | GAGGGGCGCUUGGGGUCUGG | 872 | 518 | 39.2 | 44712036 |
| gRNA192 | UUUGGCCUACGGCGACGGGA | 873 | 334 | 39.5 | 44711852 |
| gRNA230 | GAGGUUUGUGAACGCGUGGA | 874 | 500 | 39.6 | 44712018 |
| gRNA147 | CUCCUUGGUGGCCCGCCGUG | 875 | 199 | 39.7 | 44711717 |
| gRNA106 | CGCAGUGCCAGGUUAGAGAG | 876 | −118 | 39.9 | 44711422 |

TABLE 5
Exemplary Target Sequences of gRNAs Targeting CIITA

| | Chr. 16 | | gRNA Target Sequence | SEQ ID | TSS1 |
|---|---|---|---|---|---|
| gRNA No. | Strand | START | (DNA, 5′ to 3′) | NO | Distance |
| gRNA001 | + | 10876478 | GAGTGATCATATATTAGAAG | 877 | −724 |
| gRNA002 | − | 10876545 | GTCTCTGCCCATCCTCCTGG | 878 | −657 |
| gRNA003 | + | 10876605 | ATTATTTAGCATCACTTTGG | 879 | −597 |
| gRNA004 | + | 10876645 | GACTACAAGGGTACCATATT | 880 | −557 |
| gRNA005 | + | 10876738 | TATGATCTCCAGAGAAATTC | 881 | −464 |
| gRNA006 | + | 10876770 | TGTGGGAGTAGGCATGGTAG | 882 | −432 |
| gRNA007 | + | 10876831 | CTTGTCTTGAGGCATCTGGG | 883 | −371 |
| gRNA008 | − | 10876872 | ATCTGCCACCTTCTGCAGGA | 884 | −330 |
| gRNA009 | − | 10876962 | TTGCACAGAAAGTCTGTTGG | 885 | −240 |
| gRNA010 | + | 10877023 | TGAAATTAATTTCAGAGGTG | 886 | −179 |
| gRNA011 | + | 10877056 | AGGGAGTGTGGTAAAATTAG | 887 | −146 |
| gRNA012 | − | 10876520 | TCAAGGAGGAAGTCTCAGCT | 888 | −682 |
| gRNA013 | + | 10876548 | CCAGGAGGATGGGCAGAGAC | 889 | −654 |
| gRNA014 | + | 10876606 | TTATTTAGCATCACTTTGGC | 890 | −596 |
| gRNA015 | + | 10876646 | ACTACAAGGGTACCATATTT | 891 | −556 |
| gRNA016 | + | 10876739 | ATGATCTCCAGAGAAATTCA | 892 | −463 |
| gRNA017 | + | 10876801 | ATCTAAGAAGTCCCCAGCAG | 893 | −401 |
| gRNA018 | + | 10876834 | GTCTTGAGGCATCTGGGCGG | 894 | −368 |
| gRNA019 | − | 10876876 | CAATATCTGCCACCTTCTGC | 895 | −326 |
| gRNA020 | − | 10876963 | GTTGCACAGAAAGTCTGTTG | 896 | −239 |
| gRNA021 | + | 10877024 | GAAATTAATTTCAGAGGTGT | 897 | −178 |
| gRNA022 | + | 10877058 | GGGAGTGTGGTAAAATTAGA | 898 | −144 |
| gRNA023 | + | 10876530 | ACTTCCTCCTTGATACCACC | 899 | −672 |
| gRNA024 | − | 10876548 | CCAGTCTCTGCCCATCCTCC | 900 | −654 |
| gRNA025 | + | 10876632 | CCCCAAACCATCTGACTACA | 901 | −570 |
| gRNA026 | − | 10876658 | AAAGAGTGTTAACCCAAATA | 902 | −544 |
| gRNA027 | − | 10876746 | CAAATTGCCCTGAATTTCTC | 903 | −456 |
| gRNA028 | − | 10876812 | CAAGCTGAGAGCCTCTGCTG | 904 | −390 |
| gRNA029 | + | 10876835 | TCTTGAGGCATCTGGGCGGA | 905 | −367 |
| gRNA030 | + | 10876877 | CTGCAGAAGGTGGCAGATAT | 906 | −325 |
| gRNA031 | − | 10876964 | AGTTGCACAGAAAGTCTGTT | 907 | −238 |
| gRNA032 | + | 10877025 | AAATTAATTTCAGAGGTGTG | 908 | −177 |
| gRNA033 | + | 10877086 | GAAACAGAAATCTGACCGCT | 909 | −116 |
| gRNA034 | + | 10876533 | TCCTCCTTGATACCACCAGG | 910 | −669 |
| gRNA035 | + | 10876574 | AGACACTAACTTTCTCCCTA | 911 | −628 |
| gRNA036 | − | 10876632 | CCTTGTAGTCAGATGGTTTG | 912 | −570 |
| gRNA037 | + | 10876661 | TATTTGGGTTAACACTCTTT | 913 | −541 |
| gRNA038 | + | 10876747 | CAGAGAAATTCAGGGCAATT | 914 | −455 |
| gRNA039 | − | 10876813 | ACAAGCTGAGAGCCTCTGCT | 915 | −389 |
| gRNA040 | + | 10876847 | TGGGCGGAGGGCTATGATAC | 916 | −355 |
| gRNA041 | + | 10876884 | AGGTGGCAGATATTGGCAGC | 917 | −318 |
| gRNA042 | − | 10876965 | AAGTTGCACAGAAAGTCTGT | 918 | −237 |
| gRNA043 | + | 10877028 | TTAATTTCAGAGGTGTGGGG | 919 | −174 |
| gRNA044 | + | 10877087 | AAACAGAAATCTGACCGCTT | 920 | −115 |
| gRNA045 | − | 10876534 | TCCTCCTGGTGGTATCAAGG | 921 | −668 |
| gRNA046 | + | 10876575 | GACACTAACTTTCTCCCTAT | 922 | −627 |
| gRNA047 | + | 10876633 | CCCAAACCATCTGACTACAA | 923 | −569 |
| gRNA048 | + | 10876706 | TGTCTTGGGATGAAAATGAC | 924 | −496 |
| gRNA049 | + | 10876752 | AAATTCAGGGCAATTTGGTG | 925 | −450 |
| gRNA050 | − | 10876814 | GACAAGCTGAGAGCCTCTGC | 926 | −388 |
| gRNA051 | + | 10876864 | TACTGGCCCCATCCTGCAGA | 927 | −338 |
| gRNA052 | + | 10876895 | ATTGGCAGCTGGCACCAGTG | 928 | −307 |
| gRNA053 | + | 10876997 | ACCAAATTCAGTCCACAGTA | 929 | −205 |
| gRNA054 | + | 10877029 | TAATTTCAGAGGTGTGGGGA | 930 | −173 |
| gRNA055 | + | 10877088 | AACAGAAATCTGACCGCTTG | 931 | −114 |
| gRNA056 | − | 10876537 | CCATCCTCCTGGTGGTATCA | 932 | −665 |
| gRNA057 | − | 10876589 | AAATAATACTGACTCCCATA | 933 | −613 |
| gRNA058 | − | 10876633 | CCCTTGTAGTCAGATGGTTT | 934 | −569 |
| gRNA059 | + | 10876709 | CTTGGGATGAAAATGACAGG | 935 | −493 |
| gRNA060 | + | 10876753 | AATTCAGGGCAATTTGGTGT | 936 | −449 |
| gRNA061 | + | 10876820 | GAGGCTCTCAGCTTGTCTTG | 937 | −382 |
| gRNA062 | + | 10876867 | TGGCCCCATCCTGCAGAAGG | 938 | −335 |
| gRNA063 | − | 10876909 | TGATCACAATGGAACCGCAC | 939 | −293 |
| gRNA064 | − | 10877002 | TCCTTACTGTGGACTGAATT | 940 | −200 |
| gRNA065 | + | 10877036 | AGAGGTGTGGGGAGGGCTTA | 941 | −166 |
| gRNA066 | + | 10877100 | ACCGCTTGGGGCCACCTTGC | 942 | −102 |
| gRNA067 | + | 10876537 | CCTTGATACCACCAGGAGGA | 943 | −665 |
| gRNA068 | − | 10876590 | TAAATAATACTGACTCCCAT | 944 | −612 |
| gRNA069 | − | 10876634 | ACCCTTGTAGTCAGATGGTT | 945 | −568 |
| gRNA070 | + | 10876710 | TTGGGATGAAAATGACAGGT | 946 | −492 |
| gRNA071 | + | 10876759 | GGGCAATTTGGTGTGGGAGT | 947 | −443 |
| gRNA072 | + | 10876827 | TCAGCTTGTCTTGAGGCATC | 948 | −375 |
| gRNA073 | − | 10876870 | CTGCCACCTTCTGCAGGATG | 949 | −332 |
| gRNA074 | − | 10876920 | TTCAGAAATGATGATCACAA | 950 | −282 |
| gRNA075 | − | 10877009 | TTAATTTCACTTCCTTACTG | 951 | −193 |
| gRNA076 | + | 10877037 | GAGGTGTGGGGAGGGCTTAA | 952 | −165 |
| gRNA077 | + | 10876538 | CTTGATACCACCAGGAGGAT | 953 | −664 |
| gRNA078 | + | 10876602 | AGTATTATTTAGCATCACTT | 954 | −600 |
| gRNA079 | − | 10876639 | ATGGTACCCTTGTAGTCAGA | 955 | −563 |
| gRNA080 | − | 10876733 | ATTTCTCTGGAGATCATAAG | 956 | −469 |
| gRNA081 | + | 10876764 | ATTTGGTGTGGGAGTAGGCA | 957 | −438 |
| gRNA082 | + | 10876828 | CAGCTTGTCTTGAGGCATCT | 958 | −374 |
| gRNA083 | − | 10876871 | TCTGCCACCTTCTGCAGGAT | 959 | −331 |
| gRNA084 | + | 10876937 | TCTGAACGTCAGACTGTTGA | 960 | −265 |
| gRNA085 | + | 10877018 | GGAAGTGAAATTAATTTCAG | 961 | −184 |
| gRNA086 | + | 10877044 | GGGGAGGGCTTAAGGGAGTG | 962 | −158 |
| gRNA087 | − | 10877105 | CCCTGCAAGGTGGCCCCAAG | 963 | −97 |
| gRNA088 | + | 10877157 | TGCATGTTGGCTTAGCTTGG | 964 | −45 |
| gRNA089 | + | 10877220 | GAGGCTGTGTGCTTCTGAG | 795 | 18 |
| gRNA090 | + | 10877251 | GGCATCCTTGGGGAAGCTGA | 965 | 49 |
| gRNA091 | − | 10877289 | AGGCAGCAGCTCCCGGAGTC | 966 | 87 |
| gRNA092 | + | 10877336 | TGCCTGGCTCCACGCCCTGC | 967 | 134 |
| gRNA093 | + | 10877368 | GTCAGAGCCCCAAGGTAAAA | 968 | 166 |
| gRNA094 | + | 10877448 | ACAGAGAAACCATTCTGAAT | 969 | 246 |
| gRNA095 | + | 10877462 | CTGAATTGGGGATGGGGGTG | 970 | 260 |
| gRNA096 | + | 10877503 | GTCCTGCTGGGGCAGGCCAT | 971 | 301 |
| gRNA097 | − | 10877554 | GACCTGAAGTCTCCCTCCGG | 972 | 352 |
| gRNA098 | + | 10877101 | CCGCTTGGGGCCACCTTGCA | 973 | −101 |
| gRNA099 | + | 10877158 | GCATGTTGGCTTAGCTTGGC | 974 | −44 |
| gRNA100 | + | 10877220 | AGGCTGTGTGCTTCTGAGCT | 975 | 18 |
| gRNA101 | − | 10877256 | TCGTGCCCTCAGCTTCCCCA | 976 | 54 |
| gRNA102 | + | 10877291 | AGACTCCGGGAGCTGCTGCC | 977 | 89 |
| gRNA103 | + | 10877338 | GCCTGGCTCCACGCCCTGCT | 978 | 136 |
| gRNA104 | + | 10877372 | GAGCCCCAAGGTAAAAAGGC | 979 | 170 |
| gRNA105 | + | 10877449 | CAGAGAAACCATTCTGAATT | 980 | 247 |
| gRNA106 | + | 10877466 | ATTGGGGATGGGGGTGAGGA | 981 | 264 |
| gRNA107 | − | 10877505 | TTCCAATGGCCTGCCCCAGC | 982 | 303 |
| gRNA108 | − | 10877557 | GCTGACCTGAAGTCTCCCTC | 983 | 355 |
| gRNA109 | − | 10877111 | AAAAAACTCTCCCTGCAAGG | 984 | −91 |
| gRNA110 | + | 10877169 | TAGCTTGGCGGGCTCCCAAC | 985 | −33 |
| gRNA111 | + | 10877230 | CTTCTGAGCTGGGCATCCGA | 986 | 28 |
| gRNA112 | + | 10877258 | TTGGGGAAGCTGAGGGCACG | 987 | 56 |
| gRNA113 | + | 10877295 | TCCGGGAGCTGCTGCCTGGC | 988 | 93 |
| gRNA114 | − | 10877338 | ACCCAGCAGGGCGTGGAGCC | 989 | 136 |
| gRNA115 | + | 10877373 | AGCCCCAAGGTAAAAAGGCC | 990 | 171 |
| gRNA116 | + | 10877450 | AGAGAAACCATTCTGAATTG | 991 | 248 |
| gRNA117 | + | 10877467 | TTGGGGATGGGGGTGAGGAT | 992 | 265 |
| gRNA118 | − | 10877519 | AACTCTTTCACATCTTCCAA | 993 | 317 |
| gRNA119 | + | 10877561 | AGGGAGACTTCAGGTCAGCC | 994 | 359 |
| gRNA120 | − | 10877114 | TCAAAAAAACTCTCCCTGCA | 995 | −88 |
| gRNA121 | + | 10877177 | GCGGGCTCCCAACTGGTGAC | 996 | −25 |
| gRNA122 | + | 10877239 | TGGGCATCCGAAGGCATCCT | 997 | 37 |
| gRNA123 | + | 10877261 | GGGAAGCTGAGGGCACGAGG | 998 | 59 |
| gRNA124 | − | 10877296 | CCCAGCCAGGCAGCAGCTCC | 999 | 94 |
| gRNA125 | − | 10877345 | AGGTAGGACCCAGCAGGGCG | 1000 | 143 |
| gRNA126 | I | 10877391 | GCTAAATTAAGATGCTTTCC | 1001 | 189 |
| gRNA127 | + | 10877454 | AAACCATTCTGAATTGGGGA | 1002 | 252 |
| gRNA128 | + | 10877473 | ATGGGGGTGAGGATGGGAAC | 1003 | 271 |
| gRNA129 | + | 10877538 | AGTTGTCTATTTCCTTCCAC | 1004 | 336 |
| gRNA130 | + | 10877568 | CTTCAGGTCAGCCAGGTGTC | 1005 | 366 |
| gRNA131 | − | 10877141 | ACATGCAAAGAAACAAGTGA | 1006 | −61 |
| gRNA132 | − | 10877183 | TCACTAACCAGTCACCAGTT | 1007 | −19 |
| gRNA133 | + | 10877240 | GGGCATCCGAAGGCATCCTT | 1008 | 38 |
| gRNA134 | + | 10877262 | GGAAGCTGAGGGCACGAGGA | 1009 | 60 |
| gRNA135 | + | 10877296 | CCGGGAGCTGCTGCCTGGCT | 1010 | 94 |
| gRNA136 | − | 10877350 | CTGACAGGTAGGACCCAGCA | 1011 | 148 |
| gRNA137 | + | 10877407 | ATTTAGCGTGCAGTCTCAGC | 1012 | 205 |
| gRNA138 | + | 10877455 | AACCATTCTGAATTGGGGAT | 1013 | 253 |
| gRNA139 | + | 10877490 | AACAGGAGTCTGTGTCCTGC | 1014 | 288 |
| gRNA140 | + | 10877541 | TGTCTATTTCCTTCCACCGG | 1015 | 339 |
| gRNA141 | − | 10877579 | TGGTTCATACTCCAGACACC | 1016 | 377 |
| gRNA142 | − | 10877142 | AACATGCAAAGAAACAAGTG | 1017 | −60 |
| gRNA143 | − | 10877184 | ATCACTAACCAGTCACCAGT | 1018 | −18 |
| gRNA144 | + | 10877241 | GGCATCCGAAGGCATCCTTG | 1019 | 39 |
| gRNA145 | + | 10877263 | GAAGCTGAGGGCACGAGGAG | 1020 | 61 |
| gRNA146 | − | 10877309 | ATTGTGTAGGAATCCCAGCC | 1021 | 107 |
| gRNA147 | − | 10877351 | TCTGACAGGTAGGACCCAGC | 1022 | 149 |
| gRNA148 | − | 10877431 | CTCTGTTTATCTGGAATGGC | 1023 | 229 |
| gRNA149 | + | 10877456 | ACCATTCTGAATTGGGGATG | 1024 | 254 |
| gRNA150 | + | 10877491 | ACAGGAGTCTGTGTCCTGCT | 1025 | 289 |
| gRNA151 | + | 10877542 | GTCTATTTCCTTCCACCGGA | 1026 | 340 |
| gRNA152 | + | 10877597 | AACCATGTATCAGCACCGAA | 1027 | 395 |
| gRNA153 | + | 10877144 | TCACTTGTTTCTTTGCATGT | 1028 | −58 |
| gRNA154 | + | 10877188 | CTGGTGACTGGTTAGTGATG | 1029 | −14 |
| gRNA155 | − | 10877246 | AGCTTCCCCAAGGATGCCTT | 1030 | 44 |
| gRNA156 | + | 10877278 | GAGGAGGGGCTGCCAGACTC | 1031 | 76 |
| gRNA157 | + | 10877320 | TTCCTACACAATGCGTTGCC | 1032 | 118 |
| gRNA158 | + | 10877360 | TCCTACCTGTCAGAGCCCCA | 1033 | 158 |
| gRNA159 | − | 10877439 | GTTTCTCTGTTTATCTGGAA | 1034 | 237 |
| gRNA160 | + | 10877457 | CCATTCTGAATTGGGGATGG | 1035 | 255 |
| gRNA161 | + | 10877492 | CAGGAGTCTGTGTCCTGCTG | 1036 | 290 |
| gRNA162 | − | 10877550 | TGAAGTCTCCCTCCGGTGGA | 1037 | 348 |
| gRNA163 | + | 10877154 | CTTTGCATGTTGGCTTAGCT | 1038 | −48 |
| gRNA164 | + | 10877200 | TAGTGATGAGGCTAGTGATG | 1039 | −2 |
| gRNA165 | + | 10877250 | AGGCATCCTTGGGGAAGCTG | 1040 | 48 |
| gRNA166 | + | 10877278 | AGGAGGGGCTGCCAGACTCC | 1041 | 76 |
| gRNA167 | − | 10877322 | AGCCAGGCAACGCATTGTGT | 1042 | 120 |
| gRNA168 | − | 10877361 | ACCTTGGGGCTCTGACAGGT | 1043 | 159 |
| gRNA169 | − | 10877440 | GAATGGTTTCTCTGTTTATC | 1044 | 238 |
| gRNA170 | − | 10877457 | CCCCCATCCCCAATTCAGAA | 1045 | 255 |
| gRNA171 | + | 10877496 | AGTCTGTGTCCTGCTGGGGC | 1046 | 294 |
| gRNA172 | + | 10877552 | TTCCACCGGAGGGAGACTTC | 1047 | 350 |
| gRNA173 | − | 10877599 | AACCTTTCGGTGCTGATACA | 1048 | 397 |
| gRNA174 | − | 10877671 | ACCTTGCTGTGGGCCGAGCC | 1049 | 469 |
| gRNA175 | − | 10877704 | CCCCTCAGTATTTACCCAAA | 1050 | 502 |
| gRNA176 | − | 10877751 | TCTGGGTATGGAGTGGAGTC | 1051 | 549 |
| gRNA177 | + | 10877787 | CAAAATTGGAGTCAGCCTTG | 1052 | 585 |
| gRNA178 | + | 10877827 | CAGCAGCTGGGGAGAGCTGG | 1053 | 625 |
| gRNA179 | − | 10877864 | AAACAACACCATTAGGAAAC | 1054 | 662 |
| gRNA180 | + | 10877885 | TTAAAAAGGGTCAGGGGACG | 1055 | 683 |
| gRNA181 | − | 10877940 | CTGCCTTCAGAGCCGGTGCC | 1056 | 738 |
| gRNA182 | + | 10878974 | GCCGCGGCCCCAGAGCTGGC | 1057 | 1772 |
| gRNA183 | + | 10879002 | GAGGCCACCAGCAGCGCGCG | 1058 | 1800 |
| gRNA184 | − | 10877612 | GTCTGACTTCTAGAACCTTT | 1059 | 410 |
| gRNA185 | − | 10877681 | AGGCGAGAAGACCTTGCTGT | 1060 | 479 |
| gRNA186 | + | 10877704 | CCCTTTGGGTAAATACTGAG | 1061 | 502 |
| gRNA187 | − | 10877758 | CTGCTTCTCTGGGTATGGAG | 1062 | 556 |
| gRNA188 | − | 10877802 | GGCTCAACAGCTACACCTCA | 1063 | 600 |
| gRNA189 | − | 10877856 | CCATTAGGAAACTGGGGGGA | 1064 | 654 |
| gRNA190 | + | 10877871 | CCTAATGGTGTTGTTTAAAA | 1065 | 669 |
| gRNA191 | + | 10877886 | TAAAAAGGGTCAGGGGACGG | 1066 | 684 |
| gRNA192 | + | 10877945 | CACCGGCTCTGAAGGCAGCA | 1067 | 743 |
| gRNA193 | − | 10878975 | TCCCGCCAGCTCTGGGGCCG | 1068 | 1773 |
| gRNA194 | + | 10879003 | AGGCCACCAGCAGCGCGCGC | 1069 | 1801 |
| gRNA195 | + | 10877618 | GGTTCTAGAAGTCAGACTTT | 1070 | 416 |
| gRNA196 | − | 10877682 | GAGGCGAGAAGACCTTGCTG | 1071 | 480 |
| gRNA197 | − | 10877705 | ACCCCTCAGTATTTACCCAA | 1072 | 503 |
| gRNA198 | + | 10877759 | CACTCCATACCCAGAGAAGC | 1073 | 557 |
| gRNA199 | + | 10877814 | GCTGTTGAGCCCTCAGCAGC | 1074 | 612 |
| gRNA200 | + | 10877856 | CCCTCCCCCCAGTTTCCTAA | 1075 | 654 |
| gRNA201 | + | 10877872 | CTAATGGTGTTGTTTAAAAA | 1076 | 670 |
| gRNA202 | + | 10877895 | TCAGGGGACGGGGGAACAGA | 1077 | 693 |
| gRNA203 | − | 10877947 | TGCCATGCTGCCTTCAGAGC | 1078 | 745 |
| gRNA204 | + | 10878977 | GCGGCCCCAGAGCTGGCGGG | 1079 | 1775 |
| gRNA205 | − | 10879006 | GCTCCCGCGCGCGCTGCTGG | 1080 | 1804 |
| gRNA206 | + | 10877619 | GTTCTAGAAGTCAGACTTTC | 1081 | 417 |
| gRNA207 | + | 10877689 | AAGGTCTTCTCGCCTCCCTT | 1082 | 487 |
| gRNA208 | + | 10877716 | ATACTGAGGGGTGCCTCTGC | 1083 | 514 |
| gRNA209 | + | 10877760 | ACTCCATACCCAGAGAAGCA | 1084 | 558 |
| gRNA210 | + | 10877815 | CTGTTGAGCCCTCAGCAGCT | 1085 | 613 |
| gRNA211 | − | 10877857 | ACCATTAGGAAACTGGGGGG | 1086 | 655 |
| gRNA212 | + | 10877877 | GGTGTTGTTTAAAAAGGGTC | 1087 | 675 |
| gRNA213 | + | 10877898 | GGGGACGGGGGAACAGATGG | 1088 | 696 |
| gRNA214 | + | 10878933 | CGGGGAGGTAGGATGACCAG | 1089 | 1731 |
| gRNA215 | + | 10878978 | CGGCCCCAGAGCTGGCGGGA | 1090 | 1776 |
| gRNA216 | + | 10879020 | CGCGGGAGCCCGGGGAACAG | 1091 | 1818 |
| gRNA217 | + | 10877648 | GTCACTAACTCTCAGCATGC | 1092 | 446 |
| gRNA218 | + | 10877690 | AGGTCTTCTCGCCTCCCTTT | 1093 | 488 |
| gRNA219 | + | 10877720 | TGAGGGGTGCCTCTGCAGGA | 1094 | 518 |
| gRNA220 | − | 10877763 | TTTCCCTGCTTCTCTGGGTA | 1095 | 561 |
| gRNA221 | + | 10877816 | TGTTGAGCCCTCAGCAGCTG | 1096 | 614 |
| gRNA222 | − | 10877860 | AACACCATTAGGAAACTGGG | 1097 | 658 |
| gRNA223 | + | 10877878 | GTGTTGTTTAAAAAGGGTCA | 1098 | 676 |
| gRNA224 | + | 10877899 | GGGACGGGGGAACAGATGGT | 1099 | 697 |
| gRNA225 | − | 10878949 | GTCTGTGGCAGCTCGTCCGC | 1100 | 1747 |
| gRNA226 | − | 10878981 | TCTCCCTCCCGCCAGCTCTG | 1101 | 1779 |
| gRNA227 | + | 10879024 | GGAGCCCGGGGAACAGCGGT | 1102 | 1822 |
| gRNA228 | + | 10877653 | TAACTCTCAGCATGCTGGCC | 1103 | 451 |
| gRNA229 | − | 10877701 | CTCAGTATTTACCCAAAGGG | 1104 | 499 |
| gRNA230 | + | 10877721 | GAGGGGTGCCTCTGCAGGAC | 1105 | 519 |
| gRNA231 | − | 10877768 | TTTGGTTTCCCTGCTTCTCT | 1106 | 566 |
| gRNA232 | − | 10877823 | CAGCTCTCCCCAGCTGCTGA | 1107 | 621 |
| gRNA233 | − | 10877861 | CAACACCATTAGGAAACTGG | 1108 | 659 |
| gRNA234 | + | 10877879 | TGTTGTTTAAAAAGGGTCAG | 1109 | 677 |
| gRNA235 | + | 10877922 | AAGAGCACAGTGCAGACACC | 1110 | 720 |
| gRNA236 | + | 10878958 | GAGCTGCCACAGACTTGCCG | 1111 | 1756 |
| gRNA237 | − | 10878982 | CTCTCCCTCCCGCCAGCTCT | 1112 | 1780 |
| gRNA238 | − | 10879028 | GTCACCTACCGCTGTTCCCC | 1113 | 1826 |
| gRNA239 | + | 10877658 | CTCAGCATGCTGGCCTGGCT | 1114 | 456 |
| gRNA240 | + | 10877702 | CTCCCTTTGGGTAAATACTG | 1115 | 500 |
| gRNA241 | − | 10877729 | GCAGAGGTCCCGTCCTGCAG | 1116 | 527 |
| gRNA242 | + | 10877773 | AGAAGCAGGGAAACCAAAAT | 1117 | 571 |
| gRNA243 | − | 10877824 | CCAGCTCTCCCCAGCTGCTG | 1118 | 622 |
| gRNA244 | − | 10877862 | ACAACACCATTAGGAAACTG | 1119 | 660 |
| gRNA245 | + | 10877883 | GTTTAAAAAGGGTCAGGGGA | 1120 | 681 |
| gRNA246 | + | 10877928 | ACAGTGCAGACACCTGGCAC | 1121 | 726 |
| gRNA247 | + | 10878970 | ACTTGCCGCGGCCCCAGAGC | 1122 | 1768 |
| gRNA248 | + | 10878983 | CCAGAGCTGGCGGGAGGGAG | 1123 | 1781 |
| gRNA249 | + | 10877670 | GCCTGGCTCGGCCCACAGCA | 1124 | 468 |
| gRNA250 | + | 10877703 | TCCCTTTGGGTAAATACTGA | 1125 | 501 |
| gRNA251 | − | 10877745 | TATGGAGTGGAGTCTGGCAG | 1126 | 543 |
| gRNA252 | − | 10877786 | CTCAAGGCTGACTCCAATTT | 1127 | 584 |
| gRNA253 | + | 10877824 | CCTCAGCAGCTGGGGAGAGC | 1128 | 622 |
| gRNA254 | − | 10877863 | AACAACACCATTAGGAAACT | 1129 | 661 |
| gRNA255 | + | 10877884 | TTTAAAAAGGGTCAGGGGAC | 1130 | 682 |
| gRNA256 | + | 10877937 | ACACCTGGCACCGGCTCTGA | 1131 | 735 |
| gRNA257 | + | 10878973 | TGCCGCGGCCCCAGAGCTGG | 1132 | 1771 |
| gRNA258 | − | 10878983 | CCTCTCCCTCCCGCCAGCTC | 1133 | 1781 |
| gRNA259 | − | 10879029 | GGTCACCTACCGCTGTTCCC | 1134 | 1827 |
| gRNA260 | − | 10879193 | CAGAGTCGTTGCGGGGATGC | 1135 | 1991 |
| gRNA261 | + | 10879227 | GGAGCCGGGAACCCGGAGCT | 1136 | 2025 |
| gRNA262 | − | 10879050 | AGGGGTTACAGAGGAGACTT | 1137 | 1848 |
| gRNA263 | + | 10879198 | ATCCCCGCAACGACTCTGCG | 1138 | 1996 |
| gRNA264 | − | 10879231 | CAAGCCAAGCTCCGGGTTCC | 1139 | 2029 |
| gRNA265 | + | 10879054 | AGTCTCCTCTGTAACCCCTA | 1140 | 1852 |
| gRNA266 | + | 10879199 | TCCCCGCAACGACTCTGCGC | 1141 | 1997 |
| gRNA267 | − | 10879238 | GGCACAGCAAGCCAAGCTCC | 1142 | 2036 |
| gRNA268 | + | 10879058 | TCCTCTGTAACCCCTAAGGT | 1143 | 1856 |
| gRNA269 | + | 10879206 | AACGACTCTGCGCGGGAACC | 1144 | 2004 |
| gRNA270 | − | 10879239 | GGGCACAGCAAGCCAAGCTC | 1145 | 2037 |
| gRNA271 | − | 10879059 | CCCGACCTTAGGGGTTACAG | 1146 | 1857 |
| gRNA272 | + | 10879212 | TCTGCGCGGGAACCAGGAGC | 1147 | 2010 |
| gRNA273 | + | 10879249 | GCTTGCTGTGCCCAGAGCTC | 1148 | 2047 |
| gRNA274 | + | 10879059 | CCTCTGTAACCCCTAAGGTC | 1149 | 1857 |
| gRNA275 | + | 10879213 | CTGCGCGGGAACCAGGAGCC | 1150 | 2011 |
| gRNA276 | − | 10879175 | GCTGGAAACGAGGTGTTTCC | 1151 | 1973 |
| gRNA277 | + | 10879220 | GGAACCAGGAGCCGGGAACC | 1152 | 2018 |
| gRNA278 | − | 10879185 | TTGCGGGGATGCTGGAAACG | 1153 | 1983 |
| gRNA279 | − | 10879224 | AGCTCCGGGTTCCCGGCTCC | 1154 | 2022 |

TABLE 6
Exemplary Target Sequences of gRNAs Targeting TRAC

| | Chr. | | | SEQ | |
|---|---|---|---|---|---|
| | 14 | | gRNA Target Sequence | ID | TSS |
| Target No. | Strand | START | (DNA, 5′ to 3′) | NO | Distance |
| TAR1172 | − | 22547530 | AGAGTCTCTCAGCTGGTACA | 1155 | 24 |
| TAR1327 | − | 22546514 | AGGAAACAGAGGTTTGCCGG | 1156 | −992 |
| TAR1328 | − | 22546517 | AGGAGGAAACAGAGGTTTGC | 1157 | −989 |
| TAR1329 | + | 22546524 | ACCTCTGTTTCCTCCTCAAA | 1158 | −982 |
| TAR1330 | − | 22546525 | GCCTTTTGAGGAGGAAACAG | 1159 | −981 |
| TAR1331 | − | 22546534 | CGACCTCCTGCCTTTTGAGG | 1160 | −972 |
| TAR1332 | − | 22546537 | TTCCGACCTCCTGCCTTTTG | 1161 | −969 |
| TAR1333 | − | 22546597 | TCATTCTTCAGTATTTCCGT | 1162 | −909 |
| TAR1334 | + | 22546612 | AAGAATGAGTCTCAGCACTA | 1163 | −894 |
| TAR1335 | − | 22546640 | CAGAAAGCAGGAGCTGCTGG | 1164 | −866 |
| TAR1336 | − | 22546643 | CCTCAGAAAGCAGGAGCTGC | 1165 | −863 |
| TAR1337 | + | 22546643 | CCAGCAGCTCCTGCTTTCTG | 1166 | −863 |
| TAR1338 | + | 22546644 | CAGCAGCTCCTGCTTTCTGA | 1167 | −862 |
| TAR1339 | + | 22546650 | CTCCTGCTTTCTGAGGGTGA | 1168 | −856 |
| TAR1340 | − | 22546652 | ATCCTTCACCCTCAGAAAGC | 1169 | −854 |
| TAR1341 | + | 22546663 | AGGGTGAAGGATAGACGCTG | 1170 | −843 |
| TAR1342 | + | 22546694 | GACTCACTAGCACTCTATCA | 1171 | −812 |
| TAR1343 | + | 22546705 | ACTCTATCACGGCCATATTC | 1172 | −801 |
| TAR1344 | + | 22546709 | TATCACGGCCATATTCTGGC | 1173 | −797 |
| TAR1345 | + | 22546710 | ATCACGGCCATATTCTGGCA | 1174 | −796 |
| TAR1346 | − | 22546717 | CCACTGACCCTGCCAGAATA | 1175 | −789 |
| TAR1347 | + | 22546717 | CCATATTCTGGCAGGGTCAG | 1176 | −789 |
| TAR1348 | + | 22546738 | GGCTCCAACTAACATTTGTT | 1177 | −768 |
| TAR1349 | − | 22546742 | AGTACCAAACAAATGTTAGT | 1178 | −764 |
| TAR1350 | + | 22546772 | TTATTAAATAGATGTTTATA | 1179 | −734 |
| TAR1351 | + | 22546805 | ATTTCTTTCTCAGAAGAGCC | 1180 | −701 |
| TAR1352 | + | 22546810 | TTTCTCAGAAGAGCCTGGCT | 1181 | −696 |
| TAR1353 | + | 22546814 | TCAGAAGAGCCTGGCTAGGA | 1182 | −692 |
| TAR1354 | + | 22546817 | GAAGAGCCTGGCTAGGAAGG | 1183 | −689 |
| TAR1355 | − | 22546823 | CCTCATCCACCTTCCTAGCC | 1184 | −683 |
| TAR1356 | + | 22546823 | CCTGGCTAGGAAGGTGGATG | 1185 | −683 |
| TAR1357 | + | 22546843 | AGGCACCATATTCATTTTGC | 1186 | −663 |
| TAR1358 | + | 22546864 | GGTGAAATTCCTGAGATGTA | 1187 | −642 |
| TAR1359 | − | 22546873 | ACAGCAGCTCCTTACATCTC | 1188 | −633 |
| TAR1360 | + | 22546886 | GAGCTGCTGTGACTTGCTCA | 1189 | −620 |
| TAR1361 | + | 22546905 | AAGGCCTTATATCGAGTAAA | 1190 | −601 |
| TAR1362 | − | 22546909 | ACTACCGTTTACTCGATATA | 1191 | −597 |
| TAR1363 | + | 22546914 | TATCGAGTAAACGGTAGTGC | 1192 | −592 |
| TAR1364 | + | 22546915 | ATCGAGTAAACGGTAGTGCT | 1193 | −591 |
| TAR1365 | + | 22546916 | TCGAGTAAACGGTAGTGCTG | 1194 | −590 |
| TAR1366 | + | 22546928 | TAGTGCTGGGGCTTAGACGC | 1195 | −578 |
| TAR1367 | − | 22546973 | GATTGCTCTCTCATTGATAG | 1196 | −533 |
| TAR1368 | + | 22546979 | TCAATGAGAGAGCAATCTCC | 1197 | −527 |
| TAR1369 | − | 22546997 | GGGAAATCTATCACATTACC | 1198 | −509 |
| TAR1370 | − | 22547017 | TGGTATGTTGGCATTAAGTT | 1199 | −489 |
| TAR1371 | − | 22547018 | ATGGTATGTTGGCATTAAGT | 1200 | −488 |
| TAR1372 | − | 22547029 | ATGGGAGGTTTATGGTATGT | 1201 | −477 |
| TAR1373 | − | 22547037 | TTAGCAGAATGGGAGGTTTA | 1202 | −469 |
| TAR1374 | − | 22547044 | CTGGGCATTAGCAGAATGGG | 1203 | −462 |
| TAR1375 | − | 22547047 | AGGCTGGGCATTAGCAGAAT | 1204 | −459 |
| TAR1376 | − | 22547048 | TAGGCTGGGCATTAGCAGAA | 1205 | −458 |
| TAR1377 | + | 22547054 | TGCTAATGCCCAGCCTAAGT | 1206 | −452 |
| TAR1378 | + | 22547055 | GCTAATGCCCAGCCTAAGTT | 1207 | −451 |
| TAR1379 | + | 22547056 | CTAATGCCCAGCCTAAGTTG | 1208 | −450 |
| TAR1380 | − | 22547062 | TGGTCTCCCCAACTTAGGCT | 1209 | −444 |
| TAR1381 | − | 22547063 | GTGGTCTCCCCAACTTAGGC | 1210 | −443 |
| TAR1382 | − | 22547067 | TGGAGTGGTCTCCCCAACTT | 1211 | −439 |
| TAR1383 | − | 22547082 | GTACATCTTGGAATCTGGAG | 1212 | −424 |
| TAR1384 | − | 22547087 | AAACTGTACATCTTGGAATC | 1213 | −419 |
| TAR1385 | | 22547094 | GCAAAGCAAACTGTACATCT | 1214 | −412 |
| TAR1386 | + | 22547097 | AGATGTACAGTTTGCTTTGC | 1215 | −409 |
| TAR1387 | + | 22547098 | GATGTACAGTTTGCTTTGCT | 1216 | −408 |
| TAR1388 | − | 22547128 | GGCAGAGTAAAGGCAGGCAT | 1217 | −378 |
| TAR1389 | − | 22547129 | TGGCAGAGTAAAGGCAGGCA | 1218 | −377 |
| TAR1390 | − | 22547134 | AACTCTGGCAGAGTAAAGGC | 1221 | −372 |
| TAR1391 | − | 22547138 | ATATAACTCTGGCAGAGTAA | 1222 | −368 |
| TAR1392 | + | 22547144 | CTCTGCCAGAGTTATATTGC | 1223 | −362 |
| TAR1393 | + | 22547145 | TCTGCCAGAGTTATATTGCT | 1224 | −361 |
| TAR1394 | + | 22547146 | CTGCCAGAGTTATATTGCTG | 1225 | −360 |
| TAR1395 | − | 22547149 | AAACCCCAGCAATATAACTC | 1226 | −357 |
| TAR1396 | − | 22547182 | TGCTTATTCTTTTATTTAAT | 1227 | −324 |
| TAR1397 | + | 22547210 | ATTAAGTAGCCCTGCATTTC | 1228 | −296 |
| TAR1398 | − | 22547219 | TCAAGGAAACCTGAAATGCA | 1229 | −287 |
| TAR1399 | − | 22547220 | CTCAAGGAAACCTGAAATGC | 1230 | −286 |
| TAR1400 | + | 22547223 | GCATTTCAGGTTTCCTTGAG | 1231 | −283 |
| TAR1401 | + | 22547227 | TTCAGGTTTCCTTGAGTGGC | 1232 | −279 |
| TAR1402 | + | 22547232 | GTTTCCTTGAGTGGCAGGCC | 1233 | −274 |
| TAR1403 | − | 22547236 | CAGGCCTGGCCTGCCACTCA | 1234 | −270 |
| TAR1404 | + | 22547237 | CTTGAGTGGCAGGCCAGGCC | 1235 | −269 |
| TAR1405 | − | 22547250 | TGAACGTTCACGGCCAGGCC | 1236 | −256 |
| TAR1406 | − | 22547255 | TTCAGTGAACGTTCACGGCC | 1237 | −251 |
| TAR1407 | − | 22547260 | ATGATTTCAGTGAACGTTCA | 1238 | −246 |
| TAR1408 | + | 22547270 | TCACTGAAATCATGGCCTCT | 1239 | −236 |
| TAR1409 | − | 22547285 | AGCTATCAATCTTGGCCAAG | 1240 | −221 |
| TAR1410 | − | 22547293 | CAGGCACAAGCTATCAATCT | 1241 | −213 |
| TAR1411 | − | 22547312 | ATGGACTGGGACTCAGGGAC | 1242 | −194 |
| TAR1412 | − | 22547317 | TCGTGATGGACTGGGACTCA | 1243 | −189 |
| TAR1413 | − | 22547318 | CTCGTGATGGACTGGGACTC | 1244 | −188 |
| TAR1414 | − | 22547325 | CCAGCTGCTCGTGATGGACT | 1245 | −181 |
| TAR1415 | + | 22547325 | CCCAGTCCATCACGAGCAGC | 1246 | −181 |
| TAR1416 | − | 22547326 | ACCAGCTGCTCGTGATGGAC | 1247 | −180 |
| TAR1417 | − | 22547331 | TAGAAACCAGCTGCTCGTGA | 1248 | −175 |
| TAR1418 | − | 22547365 | CACGGTCTCATGCTTTATAC | 1249 | −141 |
| TAR1419 | − | 22547366 | TCACGGTCTCATGCTTTATA | 1250 | −140 |
| TAR1420 | − | 22547383 | TCTGTGGGGCTGGCAAGTCA | 1251 | −123 |
| TAR1421 | − | 22547393 | AGGGCGGGGCTCTGTGGGGC | 1252 | −113 |
| TAR1422 | − | 22547397 | GACAAGGGCGGGGCTCTGTG | 1253 | −109 |
| TAR1423 | − | 22547399 | TGGACAAGGGCGGGGCTCTG | 1254 | −107 |
| TAR1424 | + | 22547406 | GCCCCGCCCTTGTCCATCAC | 1255 | −100 |
| TAR1425 | − | 22547407 | GCCAGTGATGGACAAGGGCG | 1256 | −99 |
| TAR1426 | − | 22547408 | TGCCAGTGATGGACAAGGGC | 1257 | −98 |
| TAR1427 | − | 22547409 | ATGCCAGTGATGGACAAGGG | 1258 | −97 |
| TAR1428 | − | 22547412 | CAGATGCCAGTGATGGACAA | 1259 | −94 |
| TAR1429 | − | 22547413 | CCAGATGCCAGTGATGGACA | 1260 | −93 |
| TAR1430 | + | 22547413 | CCTTGTCCATCACTGGCATC | 1261 | −93 |
| TAR1431 | − | 22547419 | TGGAGTCCAGATGCCAGTGA | 1262 | −87 |
| TAR1432 | + | 22547425 | CTGGCATCTGGACTCCAGCC | 1263 | −81 |
| TAR1433 | + | 22547426 | TGGCATCTGGACTCCAGCCT | 1264 | −80 |
| TAR1434 | + | 22547430 | ATCTGGACTCCAGCCTGGGT | 1265 | −76 |
| TAR1435 | + | 22547432 | CTGGACTCCAGCCTGGGTTG | 1266 | −74 |
| TAR1436 | − | 22547439 | CTCTTTGCCCCAACCCAGGC | 1267 | −67 |
| TAR1437 | + | 22547440 | CAGCCTGGGTTGGGGCAAAG | 1268 | −66 |
| TAR1438 | + | 22547441 | AGCCTGGGTTGGGGCAAAGA | 1269 | −65 |
| TAR1439 | − | 22547443 | TTCCCTCTTTGCCCCAACCC | 1270 | −63 |
| TAR1440 | − | 22547483 | TCTGTGGGACAAGAGGATCA | 1271 | −23 |
| TAR1441 | − | 22547484 | ATCTGTGGGACAAGAGGATC | 1272 | −22 |
| TAR1442 | − | 22547490 | CTGGATATCTGTGGGACAAG | 1273 | −16 |
| TAR1443 | − | 22547498 | TCAGGGTTCTGGATATCTGT | 1274 | −8 |
| TAR1444 | − | 22547499 | GTCAGGGTTCTGGATATCTG | 1275 | −7 |
| TAR1445 | | 22547509 | ACACGGCAGGGTCAGGGTTC | 1276 | 3 |
| TAR1446 | − | 22547516 | AGCTGGTACACGGCAGGGTC | 1277 | 10 |
| TAR1447 | − | 22547521 | CTCTCAGCTGGTACACGGCA | 1278 | 15 |
| TAR1448 | − | 22547522 | TCTCTCAGCTGGTACACGGC | 1279 | 16 |
| TAR1449 | − | 22547533 | TGGATTTAGAGTCTCTCAGC | 1280 | 27 |
| TAR1450 | + | 22547596 | AACAAATGTGTCACAAAGTA | 1281 | 90 |
| TAR1451 | + | 22547671 | CTTCAAGAGCAACAGTGCTG | 1282 | 165 |
| TAR1452 | + | 22547676 | AGAGCAACAGTGCTGTGGCC | 1289 | 170 |
| TAR1453 | − | 22547694 | AAAGTCAGATTTGTTGCTCC | 1290 | 188 |
| TAR1454 | − | 22547730 | TGGAATAATGCTGTTGTTGA | 1291 | 224 |
| TAR1455 | − | 22547750 | CTGGGGAAGAAGGTGTCTTC | 1292 | 244 |
| TAR1456 | − | 22547760 | CTTACCTGGGCTGGGGAAGA | 1293 | 254 |
| TAR1457 | + | 22547761 | CTTCTTCCCCAGCCCAGGTA | 1294 | 255 |
| TAR1458 | + | 22547762 | TTCTTCCCCAGCCCAGGTAA | 1295 | 256 |
| TAR1459 | − | 22547767 | AGCTGCCCTTACCTGGGCTG | 1296 | 261 |
| TAR1460 | − | 22547768 | AAGCTGCCCTTACCTGGGCT | 1297 | 262 |
| TAR1461 | − | 22547769 | AAAGCTGCCCTTACCTGGGC | 1298 | 263 |
| TAR1462 | + | 22547771 | AGCCCAGGTAAGGGCAGCTT | 1299 | 265 |
| TAR1463 | − | 22547773 | CACCAAAGCTGCCCTTACCT | 1300 | 267 |
| TAR1464 | − | 22547774 | GCACCAAAGCTGCCCTTACC | 1301 | 268 |
| TAR1465 | + | 22547783 | GGCAGCTTTGGTGCCTTCGC | 1302 | 277 |
| TAR1466 | − | 22547796 | AGCAAGGAAACAGCCTGCGA | 1303 | 290 |
| TAR1467 | + | 22547801 | GCAGGCTGTTTCCTTGCTTC | 1304 | 295 |
| TAR1468 | + | 22547806 | CTGTTTCCTTGCTTCAGGAA | 1305 | 300 |
| TAR1469 | + | 22547811 | TCCTTGCTTCAGGAATGGCC | 1306 | 305 |
| TAR1470 | − | 22547812 | ACCTGGCCATTCCTGAAGCA | 1307 | 306 |
| TAR1471 | − | 22547829 | CCAGAGCTCTGGGCAGAACC | 1308 | 323 |
| TAR1472 | + | 22547829 | CCAGGTTCTGCCCAGAGCTC | 1309 | 323 |
| TAR1473 | − | 22547839 | ACATCATTGACCAGAGCTCT | 1310 | 333 |
| TAR1474 | − | 22547840 | GACATCATTGACCAGAGCTC | 1311 | 334 |
| TAR1475 | + | 22547867 | ACTCCTCTGATTGGTGGTCT | 1312 | 361 |
| TAR1476 | − | 22547870 | AGGCCGAGACCACCAATCAG | 1315 | 364 |
| TAR1477 | − | 22547890 | GGTTTTGGTGGCAATGGATA | 1316 | 384 |
| TAR1478 | − | 22547896 | AAAGAGGGTTTTGGTGGCAA | 1317 | 390 |
| TAR1479 | + | 22547925 | AGAAACAGTGAGCCTTGTTC | 1318 | 419 |
| TAR1480 | − | 22547937 | TTCTCTGGACTGCCAGAACA | 1319 | 431 |
| TAR1481 | + | 22547945 | TGGCAGTCCAGAGAATGACA | 1320 | 439 |
| TAR1482 | + | 22547946 | GGCAGTCCAGAGAATGACAC | 1321 | 440 |
| TAR1483 | − | 22547952 | TTTTTTCCCGTGTCATTCTC | 1322 | 446 |
| TAR1484 | + | 22547975 | GCAGATGAAGAGAAGGTGGC | 1323 | 469 |
| TAR1485 | + | 22547980 | TGAAGAGAAGGTGGCAGGAG | 1324 | 474 |
| TAR1486 | + | 22547981 | GAAGAGAAGGTGGCAGGAGA | 1325 | 475 |
| TAR1487 | + | 22547988 | AGGTGGCAGGAGAGGGCACG | 1326 | 482 |
| TAR1488 | − | 22548011 | CAGTTGGAGAGACTGAGGCT | 1327 | 505 |
| TAR1489 | − | 22548012 | TCAGTTGGAGAGACTGAGGC | 1328 | 506 |
| TAR1490 | − | 22548016 | GAACTCAGTTGGAGAGACTG | 1329 | 510 |
| TAR1491 | − | 22548027 | CAGGCAGGCAGGAACTCAGT | 1330 | 521 |
| TAR1492 | − | 22548038 | CTGAGCAAAGGCAGGCAGGC | 1331 | 532 |
| TAR1493 | − | 22548042 | CAGTCTGAGCAAAGGCAGGC | 1332 | 536 |
| TAR1494 | − | 22548046 | CAAACAGTCTGAGCAAAGGC | 1333 | 540 |
| TAR1495 | − | 22548050 | GGGGCAAACAGTCTGAGCAA | 1334 | 544 |
| TAR1496 | + | 22548066 | TTGCCCCTTACTGCTCTTCT | 1335 | 560 |
| TAR1497 | − | 22548069 | AGGCCTAGAAGAGCAGTAAG | 1336 | 563 |
| TAR1498 | − | 22548070 | GAGGCCTAGAAGAGCAGTAA | 1337 | 564 |
| TAR1499 | − | 22548071 | TGAGGCCTAGAAGAGCAGTA | 1338 | 565 |
| TAR1500 | − | 22548089 | TGGAGAAGGGGCTTAGAATG | 1339 | 583 |
| TAR1501 | − | 22548101 | GGAGAGGCAACTTGGAGAAG | 1340 | 595 |
| TAR1502 | − | 22548102 | AGGAGAGGCAACTTGGAGAA | 1341 | 596 |
| TAR1503 | − | 22548103 | AAGGAGAGGCAACTTGGAGA | 1342 | 597 |
| TAR1504 | − | 22548109 | AGAAATAAGGAGAGGCAACT | 1343 | 603 |
| TAR1505 | − | 22548117 | AGACAGGGAGAAATAAGGAG | 1344 | 611 |
| TAR1506 | − | 22548122 | TTGGCAGACAGGGAGAAATA | 1345 | 616 |
| TAR1507 | − | 22548132 | GAAAGATTTTTTGGCAGACA | 1346 | 626 |
| TAR1508 | − | 22548133 | GGAAAGATTTTTTGGCAGAC | 1347 | 627 |
| TAR1509 | − | 22548141 | GTGAGCTGGGAAAGATTTTT | 1348 | 635 |
| TAR1510 | − | 22548154 | TGAGACTGACTTAGTGAGCT | 1349 | 648 |
| TAR1511 | − | 22548155 | GTGAGACTGACTTAGTGAGC | 1350 | 649 |
| TAR1512 | − | 22548193 | CGGCACAATCAGTGATTGGT | 1351 | 687 |
| TAR1513 | − | 22548194 | CCGGCACAATCAGTGATTGG | 1352 | 688 |
| TAR1514 | + | 22548194 | CCACCAATCACTGATTGTGC | 1353 | 688 |
| TAR1515 | − | 22548197 | GTGCCGGCACAATCAGTGAT | 1354 | 691 |
| TAR1516 | + | 22548211 | TGCCGGCACATGAATGCACC | 1355 | 705 |
| TAR1517 | − | 22548213 | CACCTGGTGCATTCATGTGC | 1356 | 707 |
| TAR1518 | + | 22548222 | GAATGCACCAGGTGTTGAAG | 1357 | 716 |
| TAR1519 | + | 22548225 | TGCACCAGGTGTTGAAGTGG | 1358 | 719 |
| TAR1520 | − | 22548229 | AATTCCTCCACTTCAACACC | 1359 | 723 |
| TAR1521 | − | 22548259 | CAGATGAGGGGTGTGCCCAG | 1360 | 753 |
| TAR1522 | − | 22548274 | ACTAGAATGGTGCTTCCTCT | 1361 | 768 |
| TAR1523 | − | 22548275 | AACTAGAATGGTGCTTCCTC | 1362 | 769 |
| TAR1524 | + | 22548277 | AGAGGAAGCACCATTCTAGT | 1363 | 771 |
| TAR1525 | + | 22548278 | GAGGAAGCACCATTCTAGTT | 1364 | 772 |
| TAR1526 | + | 22548279 | AGGAAGCACCATTCTAGTTG | 1365 | 773 |
| TAR1527 | + | 22548280 | GGAAGCACCATTCTAGTTGG | 1366 | 774 |
| TAR1528 | − | 22548287 | ATGGGCTCCCCCAACTAGAA | 1367 | 781 |
| TAR1529 | + | 22548298 | GGGGGAGCCCATCTGTCAGC | 1368 | 792 |
| TAR1530 | + | 22548299 | GGGGAGCCCATCTGTCAGCT | 1369 | 793 |
| TAR1531 | − | 22548305 | ACTTTTCCCAGCTGACAGAT | 1370 | 799 |
| TAR1532 | − | 22548306 | GACTTTTCCCAGCTGACAGA | 1371 | 800 |
| TAR1533 | + | 22548324 | AAGTCCAAATAACTTCAGAT | 1372 | 818 |
| TAR1534 | − | 22548328 | CATTCCAATCTGAAGTTATT | 1373 | 822 |
| TAR1535 | + | 22548342 | ATTGGAATGTGTTTTAACTC | 1374 | 836 |
| TAR1536 | + | 22548343 | TTGGAATGTGTTTTAACTCA | 1375 | 837 |
| TAR1537 | − | 22548381 | TTCCCTGACTTTTGTCCTGA | 1376 | 875 |
| TAR1538 | + | 22548427 | TGAAGATACCAGCCCTACCA | 1377 | 921 |
| TAR1539 | + | 22548428 | GAAGATACCAGCCCTACCAA | 1378 | 922 |
| TAR1540 | + | 22548432 | ATACCAGCCCTACCAAGGGC | 1379 | 926 |
| TAR1541 | + | 22548433 | TACCAGCCCTACCAAGGGCA | 1380 | 927 |
| TAR1542 | − | 22548435 | CTCCCTGCCCTTGGTAGGGC | 1381 | 929 |
| TAR1543 | + | 22548438 | GCCCTACCAAGGGCAGGGAG | 1382 | 932 |
| TAR1544 | − | 22548439 | TCCTCTCCCTGCCCTTGGTA | 1383 | 933 |
| TAR1545 | − | 22548440 | GTCCTCTCCCTGCCCTTGGT | 1384 | 934 |
| TAR1546 | − | 22548444 | TAGGGTCCTCTCCCTGCCCT | 1385 | 938 |
| TAR1547 | + | 22548450 | GCAGGGAGAGGACCCTATAG | 1386 | 944 |
| TAR1548 | + | 22548455 | GAGAGGACCCTATAGAGGCC | 1387 | 949 |
| TAR1549 | + | 22548456 | AGAGGACCCTATAGAGGCCT | 1388 | 950 |
| TAR1550 | + | 22548461 | ACCCTATAGAGGCCTGGGAC | 1389 | 955 |
| TAR1551 | − | 22548462 | TCCTGTCCCAGGCCTCTATA | 1390 | 956 |
| TAR1552 | − | 22548463 | CTCCTGTCCCAGGCCTCTAT | 1391 | 957 |
| TAR1553 | − | 22548473 | TCTCATTGAGCTCCTGTCCC | 1392 | 967 |
| TAR1554 | + | 22548477 | GGACAGGAGCTCAATGAGAA | 1393 | 971 |

TABLE 7
Exemplary Target Sequences of gRNAs Targeting CD247

| Target | | gRNA Target Sequence | SEQ ID |
|---|---|---|---|
| No. | START | (DNA, 5′ to 3′) | NO |
| TAR2302 | 167517437 | GCTCAGGGGGATCAGCCTCG | 1394 |
| TAR2310 | 167517483 | TGGTTTTCTTCCTCATTACC | 1395 |
| TAR2318 | 167517527 | TAAAGGCTTGCAGCAAAGCT | 1396 |
| TAR2326 | 167517575 | GAGCAAGCACTCTTGTTGGC | 1397 |
| TAR2334 | 167517643 | CCCCGTGTTCTGTCAATGCC | 1398 |
| TAR2342 | 167517666 | GGCCATCTAGCCCAGCCAGA | 1399 |
| TAR2350 | 167517686 | GCCGCCTAAGGGGCAGGCTG | 1400 |
| TAR2358 | 167517721 | GCCGGGTGACGCACCATCCC | 1401 |
| TAR2366 | 167517743 | CAGTCCCAGGCGTGGGTGTG | 1402 |
| TAR2374 | 167517790 | GACACGGACGCTTGGCCATG | 1403 |
| TAR2382 | 167517839 | GGGCACAGGCAGGAAGCACC | 1404 |
| TAR2303 | 167517450 | GACAGGACAAACTGCTCAGG | 1405 |
| TAR2311 | 167517484 | GGTTTTCTTCCTCATTACCT | 1406 |
| TAR2319 | 167517540 | CAAAGCTGGGCTCCCACGAT | 1407 |
| TAR2327 | 167517576 | AGCAAGCACTCTTGTTGGCT | 1408 |
| TAR2335 | 167517644 | GCCCGGCATTGACAGAACAC | 1409 |
| TAR2343 | 167517667 | CGGCCATCTAGCCCAGCCAG | 1410 |
| TAR2351 | 167517687 | ACCCCAGCCTGCCCCTTAGG | 1411 |
| TAR2359 | 167517730 | GTGCGTCACCCGGCAGTCCC | 1412 |
| TAR2367 | 167517747 | CTGTCCCCACACCCACGCCT | 1413 |
| TAR2375 | 167517793 | ACGGACGCTTGGCCATGAGG | 1414 |
| TAR2383 | 167517840 | GGGGCACAGGCAGGAAGCAC | 1415 |
| TAR2304 | 167517451 | GGACAGGACAAACTGCTCAG | 1416 |
| TAR2312 | 167517493 | GTTTTCTAGCCCAGGTAATG | 1417 |
| TAR2320 | 167517541 | AAAGCTGGGCTCCCACGATC | 1418 |
| TAR2328 | 167517588 | TGTTGGCTGGGCGCTGCTCG | 1419 |
| TAR2336 | 167517645 | GGCCCGGCATTGACAGAACA | 1420 |
| TAR2344 | 167517674 | CTGGGCTAGATGGCCGCCTA | 1421 |
| TAR2352 | 167517690 | CTCACCCCAGCCTGCCCCTT | 1422 |
| TAR2360 | 167517735 | TCACCCGGCAGTCCCAGGCG | 1423 |
| TAR2368 | 167517748 | GCTGTCCCCACACCCACGCC | 1424 |
| TAR2376 | 167517799 | GCTTGGCCATGAGGTGGCGA | 1425 |
| TAR2384 | 167517849 | AGGAGGAGAGGGGCACAGGC | 1426 |
| TAR2305 | 167517452 | AGGACAGGACAAACTGCTCA | 1427 |
| TAR2313 | 167517497 | ATTACCTGGGCTAGAAAACA | 1428 |
| TAR2321 | 167517548 | GGCTCCCACGATCGGGAATG | 1429 |
| TAR2329 | 167517591 | TGGCTGGGCGCTGCTCGTGG | 1430 |
| TAR2337 | 167517651 | TCTGTCAATGCCGGGCCCTC | 1431 |
| TAR2345 | 167517675 | TGGGCTAGATGGCCGCCTAA | 1432 |
| TAR2353 | 167517696 | GGGCAGGCTGGGGTGAGTCA | 1433 |
| TAR2361 | 167517736 | CACCCGGCAGTCCCAGGCGT | 1434 |
| TAR2369 | 167517758 | GTGTGGGGACAGCCATGACA | 1435 |
| TAR2377 | 167517805 | ACAGCGCCTTCGCCACCTCA | 1436 |
| TAR2385 | 167517852 | GCCTGTGCCCCTCTCCTCCT | 1437 |
| TAR2306 | 167517453 | GAGGACAGGACAAACTGCTC | 1438 |
| TAR2314 | 167517500 | ACCTGGGCTAGAAAACATGG | 1439 |
| TAR2322 | 167517552 | CTCACCTCATTCCCGATCGT | 1440 |
| TAR2330 | 167517604 | CTCGTGGTGGTGACTTAACC | 1441 |
| TAR2338 | 167517655 | TCAATGCCGGGCCCTCTGGC | 1442 |
| TAR2346 | 167517676 | GGGCTAGATGGCCGCCTAAG | 1443 |
| TAR2354 | 167517703 | CTGGGGTGAGTCACGGAGCC | 1444 |
| TAR2362 | 167517738 | CACCCACGCCTGGGACTGCC | 1445 |
| TAR2370 | 167517770 | GTCTAGGCTCAGCCTTGTCA | 1446 |
| TAR2378 | 167517807 | ATGAGGTGGCGAAGGCGCTG | 1447 |
| TAR2307 | 167517463 | TTGTCCTGTCCTCTTTTCAC | 1448 |
| TAR2315 | 167517501 | TCCACCATGTTTTCTAGCCC | 1449 |
| TAR2323 | 167517553 | CCTCACCTCATTCCCGATCG | 1450 |
| TAR2331 | 167517622 | GGCAGATGAGACGATGTGCC | 1451 |
| TAR2339 | 167517656 | CAATGCCGGGCCCTCTGGCT | 1452 |
| TAR2347 | 167517680 | TAGATGGCCGCCTAAGGGGC | 1453 |
| TAR2355 | 167517704 | TGGGGTGAGTCACGGAGCCT | 1454 |
| TAR2363 | 167517739 | ACACCCACGCCTGGGACTGC | 1455 |
| TAR2371 | 167517774 | GACAAGGCTGAGCCTAGACA | 1456 |
| TAR2379 | 167517810 | AGGTGGCGAAGGCGCTGTGG | 1457 |
| TAR2308 | 167517467 | AAAACCAGTGAAAAGAGGAC | 1458 |
| TAR2316 | 167517510 | GAAAACATGGTGGAAGCTAA | 1459 |
| TAR2324 | 167517553 | CCACGATCGGGAATGAGGTG | 1460 |
| TAR2332 | 167517642 | GCCCCGTGTTCTGTCAATGC | 1461 |
| TAR2340 | 167517661 | TCTAGCCCAGCCAGAGGGCC | 1462 |
| TAR2348 | 167517684 | TGGCCGCCTAAGGGGCAGGC | 1463 |
| TAR2356 | 167517708 | GTGAGTCACGGAGCCTGGGA | 1464 |
| TAR2364 | 167517741 | GGCAGTCCCAGGCGTGGGTG | 1465 |
| TAR2372 | 167517782 | TGAGCCTAGACACGGACGCT | 1466 |
| TAR2380 | 167517821 | GCGCTGTGGAGGCTGCCACC | 1467 |
| TAR2309 | 167517472 | GGAAGAAAACCAGTGAAAAG | 1468 |
| TAR2317 | 167517526 | CTAAAGGCTTGCAGCAAAGC | 1469 |
| TAR2325 | 167517571 | TGAGGAGCAAGCACTCTTGT | 1470 |
| TAR2333 | 167517643 | CCCGGCATTGACAGAACACG | 1471 |
| TAR2341 | 167517664 | GGCCCTCTGGCTGGGCTAGA | 1472 |
| TAR2349 | 167517685 | GGCCGCCTAAGGGGCAGGCT | 1473 |
| TAR2357 | 167517720 | GCCTGGGATGGTGCGTCACC | 1474 |
| TAR2365 | 167517742 | GCAGTCCCAGGCGTGGGTGT | 1475 |
| TAR2373 | 167517786 | ATGGCCAAGCGTCCGTGTCT | 1476 |
| TAR2381 | 167517836 | CACAGGCAGGAAGCACCGGG | 1477 |
| TAR2390 | 167517866 | AGTAAGTGGGGTAACCAAGG | 1478 |
| TAR2398 | 167517889 | TGAAACACTCCAATGGGCCC | 1479 |
| TAR2406 | 167517930 | GCCAACAAAGAAGATTTTGT | 1480 |
| TAR2414 | 167517985 | AGCTTCTTTCACCCTCTGGC | 1481 |
| TAR2422 | 167518061 | GTGTTTCACAGAAGCTGGAC | 1482 |
| TAR2430 | 167518102 | GAGCAACCGAGGCAGGCCCA | 1483 |
| TAR2438 | 167518156 | CTCGAAAAATCTGTACCTGG | 1484 |
| TAR2446 | 167518229 | ACGGAGCAGATGCCAGCACC | 1485 |
| TAR2454 | 167518267 | CTCACTTGCCCATTGATTTG | 1486 |
| TAR2462 | 167518311 | GTGGAGGGTGGTGAGGGGCT | 1487 |
| TAR2470 | 167518330 | AGGGGAGTGGGTGTGGGTAG | 1488 |
| TAR2391 | 167517869 | GGGAGTAAGTGGGGTAACCA | 1489 |
| TAR2399 | 167517890 | CTGAAACACTCCAATGGGCC | 1490 |
| TAR2407 | 167517935 | AAAATCTTCTTTGTTGGCAA | 1491 |
| TAR2415 | 167517989 | CTTCAGCTTCTTTCACCCTC | 1492 |
| TAR2423 | 167518066 | AGGGAGTGTTTCACAGAAGC | 1493 |
| TAR2431 | 167518103 | AGAGCAACCGAGGCAGGCCC | 1494 |
| TAR2439 | 167518171 | CTAGAATACACAGCTCCTCC | 1499 |
| TAR2447 | 167518233 | AGCAGATGCCAGCACCGGGC | 1500 |
| TAR2455 | 167518270 | ACTTGCCCATTGATTTGAGG | 1501 |
| TAR2463 | 167518312 | AGTGGAGGGTGGTGAGGGGC | 1502 |
| TAR2471 | 167518336 | ATGTGTAGGGGAGTGGGTGT | 1503 |
| TAR2392 | 167517871 | TTGGTTACCCCACTTACTCC | 1504 |
| TAR2400 | 167517891 | CGGGCCCATTGGAGTGTTTC | 1505 |
| TAR2408 | 167517947 | GTTGGCAAAGGAAGAAAGAA | 1506 |
| TAR2416 | 167517990 | CAGAGGGTGAAAGAAGCTGA | 1507 |
| TAR2424 | 167518085 | CCAGGGTAGAGAAACGGAGA | 1508 |
| TAR2432 | 167518109 | AGCAAGAGAGCAACCGAGGC | 1509 |
| TAR2440 | 167518178 | GAGCTGTGTATTCTAGCAGC | 1510 |
| TAR2448 | 167518241 | TGAGGGGTCCGGCCCGGTGC | 1511 |
| TAR2456 | 167518271 | CTTGCCCATTGATTTGAGGA | 1512 |
| TAR2464 | 167518316 | GGGTAGTGGAGGGTGGTGAG | 1513 |
| TAR2472 | 167518337 | TATGTGTAGGGGAGTGGGTG | 1514 |
| TAR2393 | 167517872 | TGGTTACCCCACTTACTCCC | 1515 |
| TAR2401 | 167517895 | ATGGCCTGAAACACTCCAAT | 1516 |
| TAR2409 | 167517948 | TTGGCAAAGGAAGAAAGAAA | 1517 |
| TAR2417 | 167517991 | AGAGGGTGAAAGAAGCTGAA | 1518 |
| TAR2425 | 167518085 | CCCTCTCCGTTTCTCTACCC | 1519 |
| TAR2433 | 167518113 | AGGAAGCAAGAGAGCAACCG | 1520 |
| TAR2441 | 167518201 | CTCTGAACTGATGCCCAACT | 1521 |
| TAR2449 | 167518247 | GAGCAGTGAGGGGTCCGGCC | 1522 |
| TAR2457 | 167518275 | CCTGCCCTCCTCAAATCAAT | 1523 |
| TAR2465 | 167518317 | TGGGTAGTGGAGGGTGGTGA | 1524 |
| TAR2473 | 167518342 | TTGTGTATGTGTAGGGGAGT | 1525 |
| TAR2394 | 167517878 | AATGGGCCCGGGAGTAAGTG | 1526 |
| TAR2402 | 167517896 | CATGGCCTGAAACACTCCAA | 1527 |
| TAR2410 | 167517949 | TGGCAAAGGAAGAAAGAAAG | 1528 |
| TAR2418 | 167518018 | GAACGTCGCAGCACGAAACA | 1529 |
| TAR2426 | 167518086 | CCCAGGGTAGAGAAACGGAG | 1530 |
| TAR2434 | 167518133 | TGATAAGAGAGGAAGGAAGA | 1531 |
| TAR2442 | 167518210 | GATGCCCAACTCGGCTGTGA | 1532 |
| TAR2450 | 167518252 | CAAGTGAGCAGTGAGGGGTC | 1533 |
| TAR2458 | 167518275 | CCCATTGATTTGAGGAGGGC | 1534 |
| TAR2466 | 167518318 | GTGGGTAGTGGAGGGTGGTG | 1535 |
| TAR2395 | 167517879 | CAATGGGCCCGGGAGTAAGT | 1536 |
| TAR2403 | 167517914 | TTGTTGGTCAGTCAAGGTCA | 1537 |
| TAR2411 | 167517960 | GAAAGAAAGGGGCTGAGTGA | 1538 |
| TAR2419 | 167518023 | TCGCAGCACGAAACATGGCC | 1539 |
| TAR2427 | 167518086 | CCTCTCCGTTTCTCTACCCT | 1540 |
| TAR2435 | 167518140 | TTTCGAGTGATAAGAGAGGA | 1541 |
| TAR2443 | 167518214 | TGCTCCGTCACAGCCGAGTT | 1542 |
| TAR2451 | 167518257 | ATGGGCAAGTGAGCAGTGAG | 1543 |
| TAR2459 | 167518276 | TCCTGCCCTCCTCAAATCAA | 1544 |
| TAR2467 | 167518323 | TGGGTGTGGGTAGTGGAGGG | 1545 |
| TAR2396 | 167517880 | CCAATGGGCCCGGGAGTAAG | 1546 |
| TAR2404 | 167517920 | AAGATTTTGTTGGTCAGTCA | 1547 |
| TAR2412 | 167517973 | TGAGTGACGGAGCCAGCCAG | 1548 |
| TAR2420 | 167518041 | AGGATCGGAAAATGTCAGCC | 1549 |
| TAR2428 | 167518091 | GCAGGCCCAGGGTAGAGAAA | 1550 |
| TAR2436 | 167518144 | GATTTTTCGAGTGATAAGAG | 1551 |
| TAR2444 | 167518215 | CTGCTCCGTCACAGCCGAGT | 1552 |
| TAR2452 | 167518258 | AATGGGCAAGTGAGCAGTGA | 1553 |
| TAR2460 | 167518284 | TTGAGGAGGGCAGGATTTGA | 1554 |
| TAR2468 | 167518326 | GAGTGGGTGTGGGTAGTGGA | 1555 |
| TAR2397 | 167517880 | CCACTTACTCCCGGGCCCAT | 1556 |
| TAR2405 | 167517929 | ACCAACAAAATCTTCTTTGT | 1557 |
| TAR2413 | 167517974 | GAGTGACGGAGCCAGCCAGA | 1558 |
| TAR2421 | 167518056 | TCACAGAAGCTGGACAGGAT | 1559 |
| TAR2429 | 167518096 | TCTCTACCCTGGGCCTGCCT | 1560 |
| TAR2437 | 167518153 | TCACTCGAAAAATCTGTACC | 1561 |
| TAR2445 | 167518228 | GACGGAGCAGATGCCAGCAC | 1562 |
| TAR2453 | 167518259 | CAATGGGCAAGTGAGCAGTG | 1563 |
| TAR2461 | 167518310 | TGGAGGGTGGTGAGGGGCTG | 1564 |
| TAR2469 | 167518327 | GGAGTGGGTGTGGGTAGTGG | 1565 |
| TAR2478 | 167518378 | GTTCCCTGCCGTCGACACGT | 1566 |
| TAR2485 | 167518410 | TAATCGGCAACTGTGCCTGC | 1567 |
| TAR2493 | 167518470 | CCAGCCTCTTTCTGAGGGAA | 1568 |
| TAR2501 | 167518492 | GGAGGCAGAGGCTGAGGCAG | 1569 |
| TAR2509 | 167518529 | AGGAGAAAAGGAGTCTCTGC | 1570 |
| TAR2517 | 167518572 | CCCAGGAAGTGGAGGACTGT | 1571 |
| TAR2525 | 167518623 | AAGGGGGAGTGCGAATTTCT | 1572 |
| TAR2533 | 167518648 | GGTTCACAGGCAGGAAGCCC | 1573 |
| TAR2541 | 167518666 | CCTGGAGGTTTGAGGGTTTG | 1574 |
| TAR2549 | 167518859 | CATAAAATATTTGACAGTCT | 1575 |
| TAR2557 | 167518917 | ATCTGTCTCTACATTTGTCC | 1576 |
| TAR2479 | 167518381 | GGGCCGACGTGTCGACGGCA | 1577 |
| TAR2486 | 167518414 | CGGCAACTGTGCCTGCAGGA | 1578 |
| TAR2494 | 167518470 | CCTTTCCCTCAGAAAGAGGC | 1579 |
| TAR2502 | 167518495 | GGCAGAGGCTGAGGCAGCGG | 1580 |
| TAR2510 | 167518555 | TATTCTGCAGCTACCTCCCC | 1581 |
| TAR2518 | 167518573 | CCCCACAGTCCTCCACTTCC | 1582 |
| TAR2526 | 167518627 | GAAATTCGCACTCCCCCTTT | 1583 |
| TAR2534 | 167518651 | TCACAGGCAGGAAGCCCTGG | 1584 |
| TAR2542 | 167518737 | AAAAAGGAGCATTAAAACAA | 1585 |
| TAR2550 | 167518860 | CTAGACTGTCAAATATTTTA | 1586 |
| TAR2558 | 167518924 | AAATGTAGAGACAGATTCCT | 1587 |
| TAR2480 | 167518382 | AGGGCCGACGTGTCGACGGC | 1588 |
| TAR2487 | 167518420 | CTGTGCCTGCAGGATGGCCG | 1589 |
| TAR2495 | 167518471 | CTTTCCCTCAGAAAGAGGCT | 1590 |
| TAR2503 | 167518499 | GAGGCTGAGGCAGCGGTGGC | 1591 |
| TAR2511 | 167518561 | GCAGCTACCTCCCCAGGAAG | 1592 |
| TAR2519 | 167518573 | CCAGGAAGTGGAGGACTGTG | 1593 |
| TAR2527 | 167518635 | CACTCCCCCTTTCGGTTCAC | 1594 |
| TAR2535 | 167518658 | CAGGAAGCCCTGGAGGTTTG | 1595 |
| TAR2543 | 167518753 | GAATTATTTTTTTTAAAAAA | 1596 |
| TAR2551 | 167518863 | GACTGTCAAATATTTTATGG | 1597 |
| TAR2481 | 167518386 | AGGTAGGGCCGACGTGTCGA | 1598 |
| TAR2488 | 167518425 | TTTCACCGCGGCCATCCTGC | 1599 |
| TAR2496 | 167518474 | TCCCTCAGAAAGAGGCTGGG | 1600 |
| TAR2504 | 167518500 | AGGCTGAGGCAGCGGTGGCC | 1601 |
| TAR2512 | 167518564 | GCTACCTCCCCAGGAAGTGG | 1602 |
| TAR2520 | 167518596 | GTCGGCAGGTGCTTTCTCAA | 1603 |
| TAR2528 | 167518639 | CCTGCCTGTGAACCGAAAGG | 1604 |
| TAR2536 | 167518659 | AGGAAGCCCTGGAGGTTTGA | 1605 |
| TAR2544 | 167518777 | TATCTAATTCCTATCTTGAT | 1606 |
| TAR2552 | 167518867 | GTCAAATATTTTATGGTGGC | 1607 |
| TAR2482 | 167518394 | ACGTCGGCCCTACCTGTAAT | 1608 |
| TAR2489 | 167518437 | GTGGAAGGCGCTTTTCACCG | 1609 |
| TAR2497 | 167518475 | GCCTCCCAGCCTCTTTCTGA | 1610 |
| TAR2505 | 167518504 | TGAGGCAGCGGTGGCCGGGA | 1611 |
| TAR2513 | 167518568 | CAGTCCTCCACTTCCTGGGG | 1612 |
| TAR2521 | 167518599 | TTGAGAAAGCACCTGCCGAC | 1613 |
| TAR2529 | 167518639 | CCCCCTTTCGGTTCACAGGC | 1614 |
| TAR2537 | 167518664 | GCCCTGGAGGTTTGAGGGTT | 1615 |
| TAR2545 | 167518786 | TGGACTTTACCAATCAAGAT | 1616 |
| TAR2553 | 167518886 | CTGGCAAAATGACAGAACCC | 1617 |
| TAR2483 | 167518401 | ACAGTTGCCGATTACAGGTA | 1618 |
| TAR2490 | 167518452 | AAAGGACAAGATGAAGTGGA | 1619 |
| TAR2498 | 167518476 | TGCCTCCCAGCCTCTTTCTG | 1620 |
| TAR2506 | 167518509 | CAGCGGTGGCCGGGACGGTT | 1621 |
| TAR2514 | 167518571 | CCACAGTCCTCCACTTCCTG | 1622 |
| TAR2522 | 167518600 | TGAGAAAGCACCTGCCGACA | 1623 |
| TAR2530 | 167518640 | TCCTGCCTGTGAACCGAAAG | 1624 |
| TAR2538 | 167518665 | CCCAAACCCTCAAACCTCCA | 1625 |
| TAR2546 | 167518794 | GATTGGTAAAGTCCATCTCT | 1626 |
| TAR2554 | 167518899 | AGAACCCTGGATTAGAGTCC | 1627 |
| TAR2277 | 167518406 | CACAGTTGCCGATTACAGGT | 1628 |
| TAR2491 | 167518456 | AGGGAAAGGACAAGATGAAG | 1629 |
| TAR2499 | 167518480 | AGAAAGAGGCTGGGAGGCAG | 1630 |
| TAR2507 | 167518517 | GCCGGGACGGTTAGGAGAAA | 1631 |
| TAR2515 | 167518571 | CCCCAGGAAGTGGAGGACTG | 1632 |
| TAR2523 | 167518610 | AATTTCTTGGCCCTGTCGGC | 1633 |
| TAR2531 | 167518641 | TTCCTGCCTGTGAACCGAAA | 1634 |
| TAR2539 | 167518665 | CCCTGGAGGTTTGAGGGTTT | 1635 |
| TAR2547 | 167518806 | AACTTGTATTTGCCTAGAGA | 1636 |
| TAR2555 | 167518903 | TTGTCCTGGACTCTAATCCA | 1637 |
| TAR2484 | 167518406 | CAGGCACAGTTGCCGATTAC | 1638 |
| TAR2492 | 167518466 | TTGTCCTTTCCCTCAGAAAG | 1639 |
| TAR2500 | 167518486 | AGGCTGGGAGGCAGAGGCTG | 1640 |
| TAR2508 | 167518518 | TCCTTTTCTCCTAACCGTCC | 1641 |
| TAR2516 | 167518572 | CCCACAGTCCTCCACTTCCT | 1642 |
| TAR2524 | 167518614 | TGCGAATTTCTTGGCCCTGT | 1643 |
| TAR2532 | 167518642 | CTTCCTGCCTGTGAACCGAA | 1644 |
| TAR2540 | 167518666 | CCCCAAACCCTCAAACCTCC | 1645 |
| TAR2548 | 167518814 | AGGCAAATACAAGTTCTCGA | 1646 |
| TAR2556 | 167518904 | TTTGTCCTGGACTCTAATCC | 1647 |

TABLE 8
Exemplary Target Sequences of gRNAs Targeting RFX5

| | | gRNA Target Sequence | SEQ ID |
|---|---|---|---|
| gRNA No. | START | (DNA, 5′ to 3′) | NO |
| RNA2714 | 151347166 | TCCAACACAAGCAACAATAA | 1648 |
| RNA2646 | 151347209 | CTGGAGTGAAGGTGGCTACG | 1649 |
| RNA2670 | 151347238 | GCCGGCTGGGCCAGGACCTT | 1650 |
| RNA2727 | 151347015 | TGCGCTGGGCGTCAGAAACC | 1651 |
| RNA2730 | 151347232 | TGGGCCAGGACCTTGGGGCT | 1652 |
| RNA2628 | 151347237 | CCGGCTGGGCCAGGACCTTG | 1653 |
| RNA2689 | 151347217 | GGGCTGGGCTGGAGTGAAGG | 1654 |
| RNA2728 | 151347351 | TGCTGCCACCCGGAGACAGA | 1655 |
| RNA2648 | 151347233 | CTGGGCCAGGACCTTGGGGC | 1656 |
| RNA2704 | 151347149 | GTTGGAAGGCGACTGTTCAG | 1657 |
| RNA2694 | 151347001 | GGTAGCGGAACTCATGCGCT | 1658 |
| RNA2655 | 151347145 | GAAGGCGACTGTTCAGAGGT | 1659 |
| RNA2590 | 151347368 | AGAAGGCGCCGGGCTCGTAC | 1660 |
| RNA2711 | 151347126 | TAGGGAGGGGCAAGATGAGT | 1661 |
| RNA2723 | 151347465 | TGAGTCAAGAGGGGGAAGTC | 1662 |
| RNA2697 | 151347498 | GGTTAAAACTGGGGTCGGTT | 1663 |
| RNA2573 | 151347251 | AAGCAACGCACGAGCCGGCT | 1664 |
| RNA2690 | 151347435 | GGGGAGGGAAAATCCAAAGT | 1665 |
| RNA2625 | 151347237 | CCCCAAGGTCCTGGCCCAGC | 1666 |
| RNA2622 | 151347228 | CCAGCCCAGCCCCAAGGTCC | 1667 |
| RNA2615 | 151347171 | CACAAGCAACAATAAAGGTA | 1668 |
| RNA2634 | 151347140 | CGACTGTTCAGAGGTAGGGA | 1669 |
| RNA2578 | 151347172 | ACAAGCAACAATAAAGGTAA | 1670 |
| RNA2596 | 151347360 | AGCCCGGCGCCTTCTGTCTC | 1671 |
| RNA2729 | 151347033 | TGGGACCCCATTCCCCTGCC | 1672 |
| RNA2603 | 151347125 | AGGGAGGGGCAAGATGAGTT | 1673 |
| RNA2700 | 151347457 | GTCTTATTTGAGTCAAGAGG | 1674 |
| RNA2568 | 151347256 | AAATAAAGCAACGCACGAGC | 1675 |
| RNA2680 | 151347553 | GGACCGCAGGATGAAAGAAC | 1676 |
| RNA2703 | 151346916 | GTGGGGGCGCGCCTCAGGAT | 1677 |
| RNA2737 | 151347163 | TTATTGTTGCTTGTGTTGGA | 1678 |
| RNA2705 | 151347488 | GTTGGGTTTCGGTTAAAACT | 1679 |
| RNA2617 | 151347357 | CACCCGGAGACAGAAGGCGC | 1680 |
| RNA2638 | 151347356 | CGGCGCCTTCTGTCTCCGGG | 1681 |
| RNA2636 | 151347305 | CGCGATCTGCCTAGATACAC | 1682 |
| RNA2740 | 151346948 | TTCCCCCTCCATTCAATCCC | 1683 |
| RNA2595 | 151347314 | AGCAGGCCGCCCGTGTATCT | 1684 |
| RNA2661 | 151347139 | GACTGTTCAGAGGTAGGGAG | 1685 |
| RNA2685 | 151346852 | GGGAGCAGCGGGGGAAGTCG | 1686 |
| RNA2623 | 151347228 | CCAGGACCTTGGGGCTGGGC | 1687 |
| RNA2665 | 151347466 | GAGTCAAGAGGGGGAAGTCC | 1688 |
| RNA2668 | 151347331 | GCAGTGACCAATGACAGAGC | 1689 |
| RNA2570 | 151347570 | AACGGGCGCTCGGCTCCCTT | 1690 |
| RNA2582 | 151347358 | ACCCGGAGACAGAAGGCGCC | 1691 |
| RNA2574 | 151347144 | AAGGCGACTGTTCAGAGGTA | 1692 |
| RNA2667 | 151347056 | GATTCCGGGCACGTGCACAT | 1693 |
| RNA2731 | 151347020 | TGGGCGTCAGAAACCCGGCA | 1694 |
| RNA2683 | 151347477 | GGGAAGTCCGGGTTGGGTTT | 1695 |
| RNA2673 | 151347141 | GCGACTGTTCAGAGGTAGGG | 1696 |
| RNA2663 | 151347122 | GAGGGGCAAGATGAGTTGGG | 1697 |
| RNA2733 | 151347052 | TGTGCACGTGCCCGGAATCT | 1698 |
| RNA2739 | 151347222 | TTCACTCCAGCCCAGCCCCA | 1699 |
| RNA2688 | 151347021 | GGGCGTCAGAAACCCGGCAG | 1700 |
| RNA2707 | 151346970 | GTTTCTGCAAAGTGGAGCGG | 1701 |
| RNA2666 | 151347308 | GATCTGCCTAGATACACGGG | 1702 |
| RNA2736 | 151346892 | TGTTGTGGGAGATGACGTAG | 1703 |
| RNA2618 | 151347027 | CAGAAACCCGGCAGGGGAAT | 1704 |
| RNA2593 | 151346827 | AGATTAAGTAATGAGAACTT | 1705 |
| RNA2669 | 151347359 | GCCCGGCGCCTTCTGTCTCC | 1706 |
| RNA2708 | 151347484 | GTTTTAACCGAAACCCAACC | 1707 |
| RNA2589 | 151346953 | AGAAACCGGGGATTGAATGG | 1708 |
| RNA2639 | 151347324 | CGGGCGGCCTGCTCTGTCAT | 1709 |
| RNA2684 | 151347552 | GGGACCGCAGGATGAAAGAA | 1710 |
| RNA2630 | 151347304 | CCGTGTATCTAGGCAGATCG | 1711 |
| RNA2721 | 151347540 | TCTTCTCTTAAAGGGACCGC | 1712 |
| RNA2695 | 151347456 | GGTCTTATTTGAGTCAAGAG | 1713 |
| RNA2715 | 151346905 | TCCCACAACAGCCCATCCTG | 1714 |
| RNA2698 | 151347487 | GGTTGGGTTTCGGTTAAAAC | 1715 |
| RNA2706 | 151347493 | GTTTCGGTTAAAACTGGGGT | 1716 |
| RNA2678 | 151346894 | GCTGTTGTGGGAGATGACGT | 1717 |
| RNA2656 | 151346753 | GACCTCCCAGGCATATGCAA | 1718 |
| RNA2626 | 151347304 | CCGCGATCTGCCTAGATACA | 1719 |
| RNA2602 | 151347371 | AGGCGCCGGGCTCGTACCGG | 1720 |
| RNA2713 | 151347026 | TCAGAAACCCGGCAGGGGAA | 1721 |
| RNA2620 | 151347341 | CATTGGTCACTGCTGCCACC | 1722 |
| RNA2654 | 151346952 | GAAACCGGGGATTGAATGGA | 1723 |
| RNA2619 | 151347415 | CAGAATCTGATTGGTCAATA | 1724 |
| RNA2726 | 151347653 | TGCCGGAAGAGAAGAATCAA | 1725 |
| RNA2613 | 151347470 | CAAGAGGGGGAAGTCCGGGT | 1726 |
| RNA2716 | 151346747 | TCCTCACCATTGCATATGCC | 1727 |
| RNA2717 | 151347636 | TCTACGGTCCTCAGAGCTGC | 1728 |
| RNA2735 | 151347394 | TGTTACCAGGCAGACTTCAG | 1729 |
| RNA2676 | 151346965 | GCTCCACTTTGCAGAAACCG | 1730 |
| RNA2588 | 151347028 | AGAAACCCGGCAGGGGAATG | 1731 |
| RNA2693 | 151347000 | GGGTAGCGGAACTCATGCGC | 1732 |
| RNA2725 | 151346956 | TGCAGAAACCGGGGATTGAA | 1733 |
| RNA2597 | 151347239 | AGCCGGCTGGGCCAGGACCT | 1734 |
| RNA2671 | 151346906 | GCCTCAGGATGGGCTGTTGT | 1735 |
| RNA2696 | 151346917 | GGTGGGGGCGCGCCTCAGGA | 1736 |
| RNA2701 | 151346888 | GTGGGAGATGACGTAGGGGC | 1737 |
| RNA2640 | 151347497 | CGGTTAAAACTGGGGTCGGT | 1738 |
| RNA2614 | 151347532 | CAAGAGGTTCTTCTCTTAAA | 1739 |
| RNA2719 | 151347420 | TCTGATTGGTCAATAGGGGA | 1740 |
| RNA2633 | 151346786 | CGAACATATTCTGCCCTTCT | 1741 |
| RNA2569 | 151346950 | AACCGGGGATTGAATGGAGG | 1742 |
| RNA2583 | 151347167 | ACCTTTATTGTTGCTTGTGT | 1743 |
| RNA2632 | 151347531 | CCTTTAAGAGAAGAACCTCT | 1744 |
| RNA2699 | 151346969 | GGTTTCTGCAAAGTGGAGCG | 1745 |
| RNA2664 | 151346934 | GAGGGGGAAGGGAAGGAGGT | 1746 |
| RNA2674 | 151347556 | GCGCCCGTTCTTTCATCCTG | 1747 |
| RNA2660 | 151347448 | GACTCAAATAAGACCAACTT | 1748 |
| RNA2584 | 151347246 | ACGCACGAGCCGGCTGGGCC | 1749 |
| RNA2657 | 151346879 | GACGTAGGGGCCGGCGACGT | 1750 |
| RNA2608 | 151346760 | ATATGCCTGGGAGGTCAGAA | 1751 |
| RNA2607 | 151347684 | ATAACTAGACAATGATGGGT | 1752 |
| RNA2738 | 151347060 | TTCACCCATGTGCACGTGCC | 1753 |
| RNA2682 | 151347389 | GGCGGCCGCTGAAGTCTGCC | 1754 |
| RNA2571 | 151347610 | AACTGAAACACAAAAGGGGA | 1755 |
| RNA2645 | 151346974 | CTGCAAAGTGGAGCGGGGGT | 1756 |
| RNA2647 | 151347034 | CTGGGACCCCATTCCCCTGC | 1757 |
| RNA2686 | 151346945 | GGGATTGAATGGAGGGGGAA | 1758 |
| RNA2662 | 151346828 | GAGATTAAGTAATGAGAACT | 1759 |
| RNA2679 | 151347042 | GGAATGGGGTCCCAGATTCC | 1760 |
| RNA2687 | 151346872 | GGGCCGGCGACGTGGGGAGG | 1761 |
| RNA2681 | 151346935 | GGAGGGGGAAGGGAAGGAGG | 1762 |
| RNA2710 | 151347680 | TAAAATAACTAGACAATGAT | 1763 |
| RNA2604 | 151346874 | AGGGGCCGGCGACGTGGGGA | 1764 |
| RNA2722 | 151346880 | TGACGTAGGGGCCGGCGACG | 1765 |
| RNA2712 | 151346875 | TAGGGGCCGGCGACGTGGGG | 1766 |
| RNA2612 | 151347053 | ATGTGCACGTGCCCGGAATC | 1767 |
| RNA2642 | 151346765 | CGTATCCTTTCTGACCTCCC | 1768 |
| RNA2579 | 151347692 | ACAATGATGGGTAGGAGTGA | 1769 |
| RNA2621 | 151347531 | CCAAGAGGTTCTTCTCTTAA | 1770 |
| RNA2659 | 151347615 | GACTAAACTGAAACACAAAA | 1771 |
| RNA2572 | 151347471 | AAGAGGGGGAAGTCCGGGTT | 1772 |
| RNA2600 | 151347560 | AGGATGAAAGAACGGGCGCT | 1773 |
| RNA2616 | 151346751 | CACCATTGCATATGCCTGGG | 1774 |
| RNA2563 | 151346951 | AAACCGGGGATTGAATGGAG | 1775 |
| RNA2591 | 151347416 | AGAATCTGATTGGTCAATAG | 1776 |
| RNA2587 | 151347614 | ACTAAACTGAAACACAAAAG | 1777 |
| RNA2691 | 151346946 | GGGGATTGAATGGAGGGGGA | 1778 |
| RNA2598 | 151347376 | AGCGGCCGCCGGTACGAGCC | 1779 |
| RNA2624 | 151346748 | CCCAGGCATATGCAATGGTG | 1780 |
| RNA2585 | 151346878 | ACGTAGGGGCCGGCGACGTG | 1781 |
| RNA2631 | 151346748 | CCTCACCATTGCATATGCCT | 1782 |
| RNA2734 | 151347280 | TGTTACAACCATTCAGAAAA | 1783 |
| RNA2594 | 151347055 | AGATTCCGGGCACGTGCACA | 1784 |
| RNA2672 | 151347406 | GCCTGGTAACAGAATCTGAT | 1785 |
| RNA2637 | 151346966 | CGCTCCACTTTGCAGAAACC | 1786 |
| RNA2658 | 151346864 | GACGTGGGGAGGGGGAGCAG | 1787 |
| RNA2611 | 151347419 | ATCTGATTGGTCAATAGGGG | 1788 |
| RNA2566 | 151347252 | AAAGCAACGCACGAGCCGGC | 1789 |
| RNA2643 | 151346862 | CGTGGGGAGGGGGAGCAGCG | 1790 |
| RNA2609 | 151346791 | ATATTCTGCCCTTCTAGGCT | 1791 |
| RNA2732 | 151347455 | TGGTCTTATTTGAGTCAAGA | 1792 |
| RNA2635 | 151346907 | CGCCTCAGGATGGGCTGTTG | 1793 |
| RNA2644 | 151346804 | CTAGGCTTGGAAAAAAAACT | 1794 |
| RNA2692 | 151346873 | GGGGCCGGCGACGTGGGGAG | 1795 |
| RNA2724 | 151346975 | TGCAAAGTGGAGCGGGGGTG | 1796 |
| RNA2649 | 151347019 | CTGGGCGTCAGAAACCCGGC | 1797 |
| RNA2564 | 151347611 | AAACTGAAACACAAAAGGGG | 1798 |
| RNA2576 | 151346962 | AATCCCCGGTTTCTGCAAAG | 1799 |
| RNA2581 | 151347407 | ACCAATCAGATTCTGTTACC | 1800 |
| RNA2718 | 151346803 | TCTAGGCTTGGAAAAAAAAC | 1801 |
| RNA2677 | 151346869 | GCTCCCCCTCCCCACGTCGC | 1802 |
| RNA2586 | 151346863 | ACGTGGGGAGGGGGAGCAGC | 1803 |
| RNA2709 | 151346800 | GTTTTTTTTCCAAGCCTAGA | 1804 |
| RNA2606 | 151347679 | ATAAAATAACTAGACAATGA | 1805 |
| RNA2720 | 151346973 | TCTGCAAAGTGGAGCGGGGG | 1806 |
| RNA2605 | 151346980 | AGTGGAGCGGGGGTGGGGCG | 1807 |
| RNA2741 | 151346941 | TTGAATGGAGGGGGAAGGGA | 1808 |
| RNA2629 | 151346967 | CCGGTTTCTGCAAAGTGGAG | 1809 |
| RNA2610 | 151347655 | ATCCATTGATTCTTCTCTTC | 1810 |
| RNA2702 | 151346861 | GTGGGGAGGGGGAGCAGCGG | 1811 |
| RNA2627 | 151346967 | CCGCTCCACTTTGCAGAAAC | 1812 |
| RNA2599 | 151346921 | AGGAGGTGGGGGCGCGCCTC | 1813 |
| RNA2601 | 151347387 | AGGCAGACTTCAGCGGCCGC | 1814 |
| RNA2675 | 151346986 | GCGGGGGTGGGGCGGGGTAG | 1815 |
| RNA2592 | 151347616 | AGACTAAACTGAAACACAAA | 1816 |
| RNA2567 | 151346978 | AAAGTGGAGCGGGGGTGGGG | 1817 |
| RNA2580 | 151347414 | ACAGAATCTGATTGGTCAAT | 1818 |
| RNA2565 | 151347585 | AAAGAAAAGACAGTTCCAAA | 1819 |
| RNA2562 | 151347586 | AAAAGAAAAGACAGTTCCAA | 1820 |
| RNA2575 | 151346979 | AAGTGGAGCGGGGGTGGGGC | 1821 |
| RNA2641 | 151346968 | CGGTTTCTGCAAAGTGGAGC | 1822 |
| RNA2577 | 151346938 | AATGGAGGGGGAAGGGAAGG | 1823 |

TABLE 9
Exemplary Target Sequences of gRNAs Targeting RFXAP

| | | gRNA Target Sequence | SEQ ID |
|---|---|---|---|
| gRNA No. | START | (DNA, 5′ to 3′) | NO |
| RNA2855 | 36819624 | GGCGGACCTGTTAGACACTT | 1824 |
| RNA2856 | 36819566 | CCCTTCGCACAGGTACCTAA | 1825 |
| RNA2857 | 36819672 | GGCTAGTTTGGAGGATCTAG | 1826 |
| RNA2900 | 36818725 | CCGAGTTGTATTTGCATTGA | 1827 |
| RNA2901 | 36818726 | GCCGAGTTGTATTTGCATTG | 1828 |
| RNA2902 | 36818725 | CCCTCAATGCAAATACAACT | 1829 |
| RNA2903 | 36818732 | TGCAAATACAACTCGGCCAA | 1830 |
| RNA2904 | 36818737 | ATACAACTCGGCCAAAGGTG | 1831 |
| RNA2905 | 36818748 | AGAGGGATGGGCCACACCTT | 1832 |
| RNA2906 | 36818760 | ATAGTGGAGCGGAGAGGGAT | 1833 |
| RNA2907 | 36818761 | AATAGTGGAGCGGAGAGGGA | 1834 |
| RNA2908 | 36818765 | TGAAAATAGTGGAGCGGAGA | 1835 |
| RNA2909 | 36818766 | TTGAAAATAGTGGAGCGGAG | 1836 |
| RNA2910 | 36818771 | CTTATTTGAAAATAGTGGAG | 1837 |
| RNA2911 | 36818776 | GGTGGCTTATTTGAAAATAG | 1838 |
| RNA2912 | 36818794 | GCAGTCCATGAAGGCAGAGG | 1839 |
| RNA2913 | 36818797 | CTCGCAGTCCATGAAGGCAG | 1840 |
| RNA2914 | 36818789 | ATAAGCCACCTCTGCCTTCA | 1841 |
| RNA2915 | 36818803 | CACTAGCTCGCAGTCCATGA | 1842 |
| RNA2916 | 36818811 | GACTGCGAGCTAGTGACTCT | 1843 |
| RNA2917 | 36818842 | GATTTGAAGCTGGGATGCAG | 1844 |
| RNA2918 | 36818851 | GCAAGGGGAGATTTGAAGCT | 1845 |
| RNA2919 | 36818852 | TGCAAGGGGAGATTTGAAGC | 1846 |
| RNA2920 | 36818854 | AGCTTCAAATCTCCCCTTGC | 1847 |
| RNA2921 | 36818866 | CCCCACAAACTGCCTGCAAG | 1848 |
| RNA2922 | 36818867 | CCCCCACAAACTGCCTGCAA | 1849 |
| RNA2923 | 36818868 | TCCCCCACAAACTGCCTGCA | 1850 |
| RNA2924 | 36818864 | CTCCCCTTGCAGGCAGTTTG | 1851 |
| RNA2925 | 36818865 | TCCCCTTGCAGGCAGTTTGT | 1852 |
| RNA2926 | 36818866 | CCCCTTGCAGGCAGTTTGTG | 1853 |
| RNA2927 | 36818867 | CCCTTGCAGGCAGTTTGTGG | 1854 |
| RNA2928 | 36818908 | GTCTCACGACTTGTAGTGTT | 1855 |
| RNA2929 | 36818916 | TACAAGTCGTGAGACCGCTC | 1856 |
| RNA2930 | 36818930 | AAATTAGGATTGTTCCTGAG | 1857 |
| RNA2931 | 36818945 | GGAGTTCCAGTATGCAAATT | 1858 |
| RNA2932 | 36818939 | AACAATCCTAATTTGCATAC | 1859 |
| RNA2933 | 36818966 | CGGTTCAAAGTATCTTCTAC | 1860 |
| RNA2934 | 36818986 | TGATCCTATTGTATCTAAAG | 1861 |
| RNA2935 | 36818982 | TGAACCGCTTTAGATACAAT | 1862 |
| RNA2936 | 36818988 | GCTTTAGATACAATAGGATC | 1863 |
| RNA2937 | 36818996 | TACAATAGGATCAGGTAAAA | 1864 |
| RNA2938 | 36819024 | AAAACCTGCAGTGGCTGGCA | 1865 |
| RNA2939 | 36819029 | AGGTGAAAACCTGCAGTGGC | 1866 |
| RNA2940 | 36819020 | GCTGCCTTGCCAGCCACTGC | 1867 |
| RNA2941 | 36819033 | TGTCAGGTGAAAACCTGCAG | 1868 |
| RNA2942 | 36819049 | CAGGGTCTGGGGGCTGTGTC | 1869 |
| RNA2943 | 36819059 | GATTCGTAGACAGGGTCTGG | 1870 |
| RNA2944 | 36819060 | GGATTCGTAGACAGGGTCTG | 1871 |
| RNA2945 | 36819061 | GGGATTCGTAGACAGGGTCT | 1872 |
| RNA2946 | 36819062 | GGGGATTCGTAGACAGGGTC | 1873 |
| RNA2947 | 36819067 | AGCTGGGGGATTCGTAGACA | 1874 |
| RNA2948 | 36819068 | CAGCTGGGGGATTCGTAGAC | 1875 |
| RNA2949 | 36819069 | CTGTCTACGAATCCCCCAGC | 1876 |
| RNA2950 | 36819081 | CGGCCCCTTCGGCCAGCTGG | 1877 |
| RNA2951 | 36819082 | GCGGCCCCTTCGGCCAGCTG | 1878 |
| RNA2952 | 36819083 | CGCGGCCCCTTCGGCCAGCT | 1879 |
| RNA2953 | 36819084 | CCGCGGCCCCTTCGGCCAGC | 1880 |
| RNA2954 | 36819076 | CGAATCCCCCAGCTGGCCGA | 1881 |
| RNA2955 | 36819077 | GAATCCCCCAGCTGGCCGAA | 1882 |
| RNA2956 | 36819078 | AATCCCCCAGCTGGCCGAAG | 1883 |
| RNA2957 | 36819092 | ATGTGCGGCCGCGGCCCCTT | 1884 |
| RNA2958 | 36819084 | CCAGCTGGCCGAAGGGGCCG | 1885 |
| RNA2959 | 36819101 | TGACTGCGCATGTGCGGCCG | 1886 |
| RNA2960 | 36819107 | CGGGACTGACTGCGCATGTG | 1887 |
| RNA2961 | 36819111 | ACATGCGCAGTCAGTCCCGT | 1888 |
| RNA2962 | 36819126 | GGGCTGAGCGCACAACCAAC | 1889 |
| RNA2963 | 36819127 | GGGGCTGAGCGCACAACCAA | 1890 |
| RNA2964 | 36819146 | GCGCGGCGCCTCTGGGGGCG | 1891 |
| RNA2965 | 36819147 | AGCGCGGCGCCTCTGGGGGC | 1892 |
| RNA2966 | 36819148 | GAGCGCGGCGCCTCTGGGGG | 1893 |
| RNA2967 | 36819138 | GCGCTCAGCCCCGCCCCCAG | 1894 |
| RNA2968 | 36819151 | AGGGAGCGCGGCGCCTCTGG | 1895 |
| RNA2969 | 36819152 | CAGGGAGCGCGGCGCCTCTG | 1896 |
| RNA2970 | 36819153 | TCAGGGAGCGCGGCGCCTCT | 1897 |
| RNA2971 | 36819154 | GTCAGGGAGCGCGGCGCCTC | 1898 |
| RNA2972 | 36819163 | CCGCGGGCTGTCAGGGAGCG | 1899 |
| RNA2973 | 36819170 | TGTACGCCCGCGGGCTGTCA | 1900 |
| RNA2974 | 36819171 | GTGTACGCCCGCGGGCTGTC | 1901 |
| RNA2975 | 36819163 | CCGCGCTCCCTGACAGCCCG | 1902 |
| RNA2976 | 36819164 | CGCGCTCCCTGACAGCCCGC | 1903 |
| RNA2977 | 36819179 | GTCAGCGTGTGTACGCCCGC | 1904 |
| RNA2978 | 36819180 | CGTCAGCGTGTGTACGCCCG | 1905 |
| RNA2979 | 36819186 | GCGTACACACGCTGACGCGC | 1906 |
| RNA2980 | 36819192 | ACACGCTGACGCGCAGGCTG | 1907 |
| RNA2981 | 36819201 | CGCGCAGGCTGCGGTCGCGC | 1908 |
| RNA2982 | 36819210 | TGCGGTCGCGCAGGCGCAGT | 1909 |
| RNA2983 | 36819211 | GCGGTCGCGCAGGCGCAGTC | 1910 |
| RNA2984 | 36819212 | CGGTCGCGCAGGCGCAGTCG | 1911 |
| RNA2985 | 36819223 | GCGCAGTCGGGGCGCCTTCC | 1912 |
| RNA2986 | 36819237 | AAAAGGCGCCTATACCGGGA | 1913 |
| RNA2987 | 36819229 | TCGGGGCGCCTTCCCGGTAT | 1914 |
| RNA2988 | 36819241 | GGGTAAAAGGCGCCTATACC | 1915 |
| RNA2989 | 36819242 | GGGGTAAAAGGCGCCTATAC | 1916 |
| RNA2990 | 36819254 | TCAGGACACGCTGGGGTAAA | 1917 |
| RNA2991 | 36819261 | CAAAGACTCAGGACACGCTG | 1918 |
| RNA2992 | 36819262 | CCAAAGACTCAGGACACGCT | 1919 |
| RNA2993 | 36819263 | ACCAAAGACTCAGGACACGC | 1920 |
| RNA2994 | 36819272 | ACTTCGCGAACCAAAGACTC | 1921 |
| RNA2995 | 36819262 | CCCAGCGTGTCCTGAGTCTT | 1922 |
| RNA2996 | 36819281 | TTGGTTCGCGAAGTGCCGTT | 1923 |
| RNA2997 | 36819296 | TTAGCACCTGCTTGGCCTAA | 1924 |
| RNA2998 | 36819290 | GAAGTGCCGTTAGGCCAAGC | 1925 |
| RNA2999 | 36819304 | CCGGGCTTTTAGCACCTGCT | 1926 |
| RNA3000 | 36819304 | CCAAGCAGGTGCTAAAAGCC | 1927 |
| RNA3001 | 36819305 | CAAGCAGGTGCTAAAAGCCC | 1928 |
| RNA3002 | 36819306 | AAGCAGGTGCTAAAAGCCCG | 1929 |
| RNA3003 | 36819322 | TGGCCGGGGTCCACGACCCC | 1930 |
| RNA3004 | 36819312 | GTGCTAAAAGCCCGGGGTCG | 1931 |
| RNA3005 | 36819323 | CTGGCCGGGGTCCACGACCC | 1932 |
| RNA3006 | 36819319 | AAGCCCGGGGTCGTGGACCC | 1933 |
| RNA3007 | 36819324 | CGGGGTCGTGGACCCCGGCC | 1934 |
| RNA3008 | 36819336 | ATGCTGCTAAGACCTGGCCG | 1935 |
| RNA3009 | 36819337 | CATGCTGCTAAGACCTGGCC | 1936 |
| RNA3010 | 36819338 | CCATGCTGCTAAGACCTGGC | 1937 |
| RNA3011 | 36819342 | GCCTCCATGCTGCTAAGACC | 1938 |
| RNA3012 | 36819338 | CCGGCCAGGTCTTAGCAGCA | 1939 |
| RNA3013 | 36819341 | GCCAGGTCTTAGCAGCATGG | 1940 |
| RNA3014 | 36819347 | TCTTAGCAGCATGGAGGCGC | 1941 |
| RNA3015 | 36819348 | CTTAGCAGCATGGAGGCGCA | 1942 |
| RNA3016 | 36819356 | CATGGAGGCGCAGGGTGTAG | 1943 |
| RNA3017 | 36819359 | GGAGGCGCAGGGTGTAGCGG | 1944 |
| RNA3018 | 36819360 | GAGGCGCAGGGTGTAGCGGA | 1945 |
| RNA3019 | 36819365 | GCAGGGTGTAGCGGAGGGCG | 1946 |
| RNA3020 | 36819366 | CAGGGTGTAGCGGAGGGCGC | 1947 |
| RNA3021 | 36819367 | AGGGTGTAGCGGAGGGCGCG | 1948 |
| RNA3022 | 36819371 | TGTAGCGGAGGGCGCGGGGC | 1949 |
| RNA3023 | 36819372 | GTAGCGGAGGGCGCGGGGCC | 1950 |
| RNA3024 | 36819390 | GGCACGCCGCTGGCGGCGCC | 1951 |
| RNA3025 | 36819384 | GCGGGGCCGGGCGCCGCCAG | 1952 |
| RNA3026 | 36819397 | GGGGTGGGGCACGCCGCTGG | 1953 |
| RNA3027 | 36819400 | CGCGGGGTGGGGCACGCCGC | 1954 |
| RNA3028 | 36819411 | GGGGCTAGGGCCGCGGGGTG | 1955 |
| RNA3029 | 36819401 | CAGCGGCGTGCCCCACCCCG | 1956 |
| RNA3030 | 36819412 | CGGGGCTAGGGCCGCGGGGT | 1957 |
| RNA3031 | 36819413 | CCGGGGCTAGGGCCGCGGGG | 1958 |
| RNA3032 | 36819416 | CAGCCGGGGCTAGGGCCGCG | 1959 |
| RNA3033 | 36819417 | GCAGCCGGGGCTAGGGCCGC | 1960 |
| RNA3034 | 36819418 | CGCAGCCGGGGCTAGGGCCG | 1961 |
| RNA3035 | 36819413 | CCACCCCGCGGCCCTAGCCC | 1962 |
| RNA3036 | 36819424 | GGGAGCCGCAGCCGGGGCTA | 1963 |
| RNA3037 | 36819425 | TGGGAGCCGCAGCCGGGGCT | 1964 |
| RNA3038 | 36819419 | CGCGGCCCTAGCCCCGGCTG | 1965 |
| RNA3039 | 36819430 | CAAGGTGGGAGCCGCAGCCG | 1966 |
| RNA3040 | 36819431 | CCAAGGTGGGAGCCGCAGCC | 1967 |
| RNA3041 | 36819432 | GCCAAGGTGGGAGCCGCAGC | 1968 |
| RNA3042 | 36819431 | CCCGGCTGCGGCTCCCACCT | 1969 |
| RNA3043 | 36819444 | ACCGAGGCTGGCGCCAAGGT | 1970 |
| RNA3044 | 36819445 | CACCGAGGCTGGCGCCAAGG | 1971 |
| RNA3045 | 36819448 | CGCCACCGAGGCTGGCGCCA | 1972 |
| RNA3046 | 36819443 | TCCCACCTTGGCGCCAGCCT | 1973 |
| RNA3047 | 36819456 | GCCGCGGCCGCCACCGAGGC | 1974 |
| RNA3048 | 36819446 | CACCTTGGCGCCAGCCTCGG | 1975 |
| RNA3049 | 36819449 | CTTGGCGCCAGCCTCGGTGG | 1976 |
| RNA3050 | 36819460 | AGAGGCCGCGGCCGCCACCG | 1977 |
| RNA3051 | 36819455 | GCCAGCCTCGGTGGCGGCCG | 1978 |
| RNA3052 | 36819472 | CAGGGTGAATTGAGAGGCCG | 1979 |
| RNA3053 | 36819478 | CACTAGCAGGGTGAATTGAG | 1980 |
| RNA3054 | 36819490 | ACAGGGTTGCATCACTAGCA | 1981 |
| RNA3055 | 36819491 | CACAGGGTTGCATCACTAGC | 1982 |
| RNA3056 | 36819495 | CTAGTGATGCAACCCTGTGC | 1983 |
| RNA3057 | 36819496 | TAGTGATGCAACCCTGTGCT | 1984 |
| RNA3058 | 36819507 | GCCTCGTCCTGCCCAGCACA | 1985 |
| RNA3059 | 36819508 | AGCCTCGTCCTGCCCAGCAC | 1986 |
| RNA3060 | 36819500 | GATGCAACCCTGTGCTGGGC | 1987 |
| RNA3061 | 36819506 | ACCCTGTGCTGGGCAGGACG | 1988 |
| RNA3062 | 36819512 | TGCTGGGCAGGACGAGGCTG | 1989 |
| RNA3063 | 36819519 | CAGGACGAGGCTGCGGCCCC | 1990 |
| RNA3064 | 36819520 | AGGACGAGGCTGCGGCCCCC | 1991 |
| RNA3065 | 36819521 | GGACGAGGCTGCGGCCCCCG | 1992 |
| RNA3066 | 36819522 | GACGAGGCTGCGGCCCCCGG | 1993 |
| RNA3067 | 36819535 | CGCCCCAACGCTGCCCCCGG | 1994 |
| RNA3068 | 36819536 | CCGCCCCAACGCTGCCCCCG | 1995 |
| RNA3069 | 36819537 | CCCGCCCCAACGCTGCCCCC | 1996 |
| RNA3070 | 36819538 | GCCCGCCCCAACGCTGCCCC | 1997 |
| RNA3071 | 36819531 | GCGGCCCCCGGGGGCAGCGT | 1998 |
| RNA3072 | 36819532 | CGGCCCCCGGGGGCAGCGTT | 1999 |
| RNA3073 | 36819533 | GGCCCCCGGGGGCAGCGTTG | 2000 |
| RNA3074 | 36819536 | CCCCGGGGGCAGCGTTGGGG | 2001 |
| RNA3075 | 36819537 | CCCGGGGGCAGCGTTGGGGC | 2002 |
| RNA3076 | 36819550 | TTGGGGCGGGCAAGCCCGTT | 2003 |
| RNA3077 | 36819564 | CCTTCGCACAGGTACCTAAC | 2004 |
| RNA3078 | 36819564 | CCCGTTAGGTACCTGTGCGA | 2005 |
| RNA3079 | 36819575 | CATCCCCGGCCCCTTCGCAC | 2006 |
| RNA3080 | 36819565 | CCGTTAGGTACCTGTGCGAA | 2007 |
| RNA3081 | 36819566 | CGTTAGGTACCTGTGCGAAG | 2008 |
| RNA3082 | 36819570 | AGGTACCTGTGCGAAGGGGC | 2009 |
| RNA3083 | 36819571 | GGTACCTGTGCGAAGGGGCC | 2010 |
| RNA3084 | 36819572 | GTACCTGTGCGAAGGGGCCG | 2011 |
| RNA3085 | 36819576 | CTGTGCGAAGGGGCCGGGGA | 2012 |
| RNA3086 | 36819589 | AGCCTCCTCTTCGCCATCCC | 2013 |
| RNA3087 | 36819584 | AGGGGCCGGGGATGGCGAAG | 2014 |
| RNA3088 | 36819587 | GGCCGGGGATGGCGAAGAGG | 2015 |
| RNA3089 | 36819591 | GGGGATGGCGAAGAGGAGGC | 2016 |
| RNA3090 | 36819592 | GGGATGGCGAAGAGGAGGCT | 2017 |
| RNA3091 | 36819593 | GGATGGCGAAGAGGAGGCTG | 2018 |
| RNA3092 | 36819596 | TGGCGAAGAGGAGGCTGGGG | 2019 |
| RNA3093 | 36819602 | AGAGGAGGCTGGGGAGGACG | 2020 |
| RNA3094 | 36819605 | GGAGGCTGGGGAGGACGAGG | 2021 |
| RNA3095 | 36819629 | GAGGGTCCGAAGTGTCTAAC | 2022 |
| RNA3096 | 36819632 | GTTAGACACTTCGGACCCTC | 2023 |
| RNA3097 | 36819633 | TTAGACACTTCGGACCCTCC | 2024 |
| RNA3098 | 36819634 | TAGACACTTCGGACCCTCCG | 2025 |
| RNA3099 | 36819635 | AGACACTTCGGACCCTCCGG | 2026 |
| RNA3100 | 36819636 | GACACTTCGGACCCTCCGGG | 2027 |
| RNA3101 | 36819647 | CGCTCTCGCCTCCCCCCGGA | 2028 |
| RNA3102 | 36819648 | GCGCTCTCGCCTCCCCCCGG | 2029 |
| RNA3103 | 36819639 | ACTTCGGACCCTCCGGGGGG | 2030 |
| RNA3104 | 36819651 | GCCGCGCTCTCGCCTCCCCC | 2031 |
| RNA3105 | 36819650 | TCCGGGGGGAGGCGAGAGCG | 2032 |
| RNA3106 | 36819659 | AGGCGAGAGCGCGGCTAGTT | 2033 |
| RNA3107 | 36819662 | CGAGAGCGCGGCTAGTTTGG | 2034 |
| RNA3108 | 36819677 | TTTGGAGGATCTAGAGGACG | 2035 |
| RNA3109 | 36819689 | AGAGGACGAGGAGACTCACT | 2036 |
| RNA3110 | 36819690 | GAGGACGAGGAGACTCACTC | 2037 |
| RNA3111 | 36819691 | AGGACGAGGAGACTCACTCG | 2038 |
| RNA3112 | 36819692 | GGACGAGGAGACTCACTCGG | 2039 |
| RNA3113 | 36819693 | GACGAGGAGACTCACTCGGG | 2040 |

TABLE 10
Exemplary sequences of gRNAs Targeting CD3D/G

| | Gene | | gRNA Target Sequence | SEQ ID |
|---|---|---|---|---|
| gRNA No. | Targeted | START | (DNA, 5′ to 3′) | NO |
| RNA1294 | CD3D | 118340372 | ACTTCGATAATGAACTTGCA | 2041 |
| RNA1295 | CD3D | 118340445 | AAACGCATCCTGGACCCACG | 2042 |
| RNA2298 | CD3D | 118342497 | CCATCAGTAATGTCCCTCTC | 2043 |
| RNA2299 | CD3D | 118342497 | CCTGAGAGGGACATTACTGA | 2044 |
| RNA2300 | CD3D | 11834250 | GTGGGGGAAGGGACCTGAGA | 2045 |
| RNA2301 | CD3D | 118342511 | GGTGGGGGAAGGGACCTGAG | 2046 |
| RNA2302 | CD3D | 118342518 | GGTCCCTTCCCCCACCCACC | 2047 |
| RNA2303 | CD3D | 118342521 | ACTCCAGGTGGGTGGGGGAA | 2048 |
| RNA2304 | CD3D | 118342522 | TACTCCAGGTGGGTGGGGGA | 2049 |
| RNA2305 | CD3D | 118342526 | AGGCTACTCCAGGTGGGTGG | 2050 |
| RNA2306 | CD3D | 118342527 | AAGGCTACTCCAGGTGGGTG | 2051 |
| RNA2307 | CD3D | 118342528 | TAAGGCTACTCCAGGTGGGT | 2052 |
| RNA2308 | CD3D | 118342529 | GTAAGGCTACTCCAGGTGGG | 2053 |
| RNA2309 | CD3D | 118342532 | AAGGTAAGGCTACTCCAGGT | 2054 |
| RNA2310 | CD3D | 118342533 | CAAGGTAAGGCTACTCCAGG | 2055 |
| RNA2311 | CD3D | 118342536 | TCGCAAGGTAAGGCTACTCC | 2056 |
| RNA2312 | CD3D | 118342543 | TAGCCTTACCTTGCGAGAGA | 2057 |
| RNA2313 | CD3D | 118342544 | AGCCTTACCTTGCGAGAGAA | 2058 |
| RNA2314 | CD3D | 118342546 | TACCCTTCTCTCGCAAGGTA | 2059 |
| RNA2315 | CD3D | 118342551 | CTGGCTACCCTTCTCTCGCA | 2060 |
| RNA2316 | CD3D | 118342558 | AGAGAAGGGTAGCCAGTACC | 2061 |
| RNA2317 | CD3D | 118342570 | GTTTCTCTCTGGCCTGGTAC | 2062 |
| RNA2318 | CD3D | 118342576 | TAGCACGTTTCTCTCTGGCC | 2063 |
| RNA2319 | CD3D | 118342581 | GAACATAGCACGTTTCTCTC | 2064 |
| RNA2320 | CD3D | 118342594 | TGCTATGTTCCATCTCCCAG | 2065 |
| RNA2321 | CD3D | 118342603 | GGATGAGTTCCGCTGGGAGA | 2066 |
| RNA2322 | CD3D | 118342609 | TCTACTGGATGAGTTCCGCT | 2067 |
| RNA2323 | CD3D | 118342610 | ATCTACTGGATGAGTTCCGC | 2068 |
| RNA2324 | CD3D | 118342619 | CTCATCCAGTAGATAAAGCC | 2069 |
| RNA2325 | CD3D | 118342624 | GGTGACCTGGCTTTATCTAC | 2070 |
| RNA2326 | CD3D | 118342637 | AGGCTGATAGTTCGGTGACC | 2071 |
| RNA2327 | CD3D | 118342639 | AGGTCACCGAACTATCAGCC | 2072 |

TABLE 11
Exemplary sequences of gRNAs selected for multiplexing

| | | Target | | | SEQ |
|---|---|---|---|---|---|
| | Gene | Sequence | Start | | ID |
| gRNA No. | Target | ID | Position | gRNA target sequence | NO |
| RNA101 | B2M | TAR179 | 44711559 | CGCGAGCACAGCTAAGGCCA | 2073 |
| RNA110 | B2M | TAR582 | 44711267 | GCGCCCCAGCTTGGGACACC | 2074 |
| RNA2973 | RFXAP | TAR4256 | 36819170 | TGTACGCCCGCGGGCTGTCA | 1900 |
| RNA2330 | CD3D | TAR3575 | 118342657 | GGGGAGGGCAGCTCTCACCC | 2075 |
| RNA2505 | CD3G | TAR3750 | 118344343 | AGTCTAGCTGCTGCACAGGC | 2076 |

Any tracr sequence known in the art is contemplated for a gRNA described herein. In some embodiments, a gRNA described herein has a tracr sequence shown in Table 12 below, or a tracr sequence at least 70%, 75%, 80%, 85%, 90%, 95%, 96%, 97%, 98%, or 99% identical to the tracr sequence shown below (“SEQ” indicates SEQ ID NO).

TABLE 12
Exemplary TRACR Sequences

| SEQ | Sequence (5′ to 3′) |
|---|---|
| 653 | GUUUAAGAGCUAUGCUGGAAACAGCAUAGCAAGUUUAAAUAAGGCUAGUCC |
| | GUUAUCAACUUGAAAAAGUGGCACCGAGUCGGUGCUUUUUUU |
| 654 | GUUUUAGAGCUAGAAAUAGCAAGUUAAAAUAAGGCUAGUCCGUUAUCAACU |
| | UGAAAAAGUGGCACCGAGUCGGUGCUUUU |
| 655 | GUUUAAGAGCUAAGCUGGAAACAGCAUAGCAAGUUUAAAUAAGGCUAGUCC |
| | GUUAUCAACUUGAAAAAGUGGCACCGAGUCGGUGCUUUUUU |
| 656 | GUUUAAGAGCUAAGCUGGAAACAGCAUAGCAAGUUUAAAUAAGGCUAGUCC |
| | GUUAUCAACUUGAAAAAGUGGCACCGAGUCGGUGCUUUUUUU |

In some embodiments, the gRNA herein is provided to the cell directly (e.g., through an RNP complex together with the CRISPR-associated protein domain). In some embodiments, the gRNA is provided to the cell through an expression vector (e.g., a plasmid vector or a viral vector) introduced into the cell, where the cell then expresses the gRNA from the expression vector. Methods of introducing gRNAs and expression vectors into cells are well known in the art.

### III. EFFECTOR DOMAINS

Epigenetic editors described herein include one or more effector protein domains (also “epigenetic effector domains,” or “effector domains,” as used herein) that effect epigenetic modification of a target gene. An epigenetic editor with one or more effector domains may modulate expression of a target gene without altering its nucleobase sequence. In some embodiments, an effector domain described herein may provide repression or silencing of expression of a target gene such as B2M, CIITA, TRAC, RFX5, RFXAP, CD3D, and/or CD3G, e.g., by repressing transcription or by modifying or remodeling chromatin. Such effector domains are also referred to herein as “repression domains,” “repressor domains,” or “epigenetic repressor domains.” Non-limiting examples of chemical modifications that may be mediated by effector domains include methylation, demethylation, acetylation, deacetylation, phosphorylation, SUMOylation and/or ubiquitination of DNA or histone residues.

In some embodiments, an effector domain of an epigenetic editor described herein may make histone tail modifications, e.g., by adding or removing active marks on histone tails.

In some embodiments, an effector domain of an epigenetic editor described herein may comprise or recruit a transcription-related protein, e.g., a transcription repressor. The transcription-related protein may be endogenous or exogenous.

In some embodiments, an effector domain of an epigenetic editor described herein may, for example, comprise a protein that directly or indirectly blocks access of a transcription factor to the gene of interest harboring the target sequence.

An effector domain may be a full-length protein or a fragment thereof that retains the epigenetic effector function (a “functional domain”). Functional domains that are capable of modulating (e.g., repressing) gene expression can be derived from a larger protein. For example, functional domains that can reduce target gene expression may be identified based on sequences of repressor proteins. Amino acid sequences of gene expression-modulating proteins may be obtained from available genome browsers, such as the UCSD genome browser or Ensembl genome browser. Protein annotation databases such as UniProt or Pfam can be used to identify functional domains within the full protein sequence. As a starting point, the largest sequence, encompassing all regions identified by different databases, may be tested for gene expression modulation activity. Various truncations then may be tested to identify the minimal functional unit.

Variants of effector domains described herein are also contemplated by the present disclosure. A variant may, for example, refer to a polypeptide with at least about 85%, 90%, 91%, 92%, 93%, 94%, 95%, 96%, 97%, 98%, 99%, or 100% sequence identity and/or sequence similarity to a wildtype effector domain described herein. In particular embodiments, the variant retains at least about 85%, 90%, 91%, 92%, 93%, 94%, 95%, 96%, 97%, 98%, 99%, or 100% of the epigenetic effector function of the wildtype effector domain.

In some embodiments, an effector domain described herein may comprise a fusion of two or more effector domains (e.g., KOX1 KRAB and ZIM3). The effector domain may, for example, comprise a fusion of 2, 3, 4, 5, 6, 7, 8, 9, or 10 effector domains, such as effector domains described herein. In certain embodiments, an effector domain comprises a fusion of a truncated form of an effector domain and a second effector domain. In certain embodiments, an effector domain comprises a fusion of the truncated forms of two effector domains (e.g., fusions of the N- and C-terminal portions of the two effector domains).

In some embodiments, an epigenetic editor described herein may comprise 1 effector domain, 2 effector domains, 3 effector domains, 4 effector domains, 5 effector domains, 6 effector domains, 7 effector domains, 8 effector domains, 9 effector domains, 10 effector domains, or more. In certain embodiments, the epigenetic editor comprises one or more fusion proteins (e.g., one, two, or three fusion proteins), each with one or more effector domains (e.g., one, two, or three effector domains) linked to a DNA-binding domain. In some embodiments, the effector domains may induce a combination of epigenetic modifications, e.g., transcription repression and DNA methylation, DNA methylation and histone deacetylation, DNA methylation and histone demethylation, DNA methylation and histone methylation, DNA methylation and histone phosphorylation, DNA methylation and histone ubiquitylation, DNA methylation, and histone SUMOylation.

In certain embodiments, an effector domain described herein (e.g., DNMT3A and/or DNMT3L) is encoded by a nucleotide sequence as found in the native genome (e.g., human or murine) for that effector domain. In other embodiments, an effector domain described herein is encoded by a nucleotide sequence that has been codon-optimized for optimal expression in human cells.

Effector domains described herein may include, for example, transcriptional repressors, DNA methyltransferases, and/or histone modifiers, as further detailed below.

### A. Transcriptional Repressors

In some embodiments, an epigenetic effector domain described herein mediates repression of a target gene's expression (e.g., transcription). The effector domain may comprise, e.g., a Krüppel-associated box (KRAB) repressor domain, a Repressor Element Silencing Transcription Factor (REST) repressor domain, a KRAB-associated protein 1 (KAP1) domain, a MAD domain, a FKHR (forkhead in rhabdosarcoma gene) repressor domain, an EGR-1 (early growth response gene product-1) repressor domain, an ets2 repressor factor repressor domain (ERD), a MAD smSIN3 interaction domain (SID), a WRPW (SEQ ID NO: 2077) motif of the hairy-related basic helix-loop-helix (bHLH) repressor proteins, an HP1 alpha chromo-shadow repressor domain, an HP1 beta repressor domain, or any combination thereof. The effector domain may recruit one or more protein domains that repress expression of the target gene, e.g., through a scaffold protein. In some embodiments, the effector domain may recruit or interact with a scaffold protein domain that recruits a PRMT protein, a HDAC protein, a SETDB1 protein, or a NuRD protein domain. In some embodiments, the effector domain comprises a functional domain derived from a zinc finger repressor protein, such as a KRAB domain. KRAB domains are found in approximately 400 human ZFP-based transcription factors. Descriptions of KRAB domains may be found, for example, in Ecco et al., Development (2017) 144 (15): 2719-29 and Lambert et al., Cell (2018) 172:650-65.

In certain embodiments, the effector domain comprises a repressor domain (e.g., KRAB) derived from KOX1/ZNF10, KOX8/ZNF708, ZNF43, ZNF184, ZNF91, HPF4, HTF10, or HTF34. In some embodiments, the effector domain comprises a repressor domain (e.g., KRAB) derived from ZIM3, ZNF436, ZNF257, ZNF675, ZNF490, ZNF320, ZNF331, ZNF816, ZNF680, ZNF41, ZNF189, ZNF528, ZNF543, ZNF554, ZNF140, ZNF610, ZNF264, ZNF350, ZNF8, ZNF582, ZNF30, ZNF324, ZNF98, ZNF669, ZNF677, ZNF596, ZNF214, ZNF37, ZNF34, ZNF250, ZNF547, ZNF273, ZNF354, ZFP82, ZNF224, ZNF33, ZNF45, ZNF175, ZNF595, ZNF184, ZNF419, ZFP28-1, ZFP28-2, ZNF18, ZNF213, ZNF394, ZFP1, ZFP14, ZNF416, ZNF557, ZNF566, ZNF729, ZIM2, ZNF254, ZNF764, ZNF785, or any combination thereof. For example, the repressor domain may be a KRAB domain derived from KOX1, ZIM3, ZFP28, or ZN627. In particular embodiments, the repressor domain is a ZIM3 KRAB domain. In further embodiments, the effector domain is derived from a human protein, e.g., a human ZIM3, a human KOX1, a human ZFP28, or a human ZN627.

Sequences of exemplary effector domains that may reduce or silence target gene expression, or protein sequences that contain them, are provided in Table 13 below (“SEQ” indicates SEQ ID NO). Further examples of repressors and transcriptional repressor domains can be found, e.g., in PCT Patent Publication WO 2021/226077 and Tycko et al., Cell (2020) 183(7):2020-35, each of which is incorporated herein by reference in its entirety.

TABLE 13
Exemplary Effector Domains That May
Reduce or Silence Gene Expression

| Protein | SEQ |
|---|---|
| ZIM3 | 33 |
| ZNF436 | 34 |
| ZNF257 | 35 |
| ZNF675 | 36 |
| ZNF490 | 37 |
| ZNF320 | 38 |
| ZNF331 | 39 |
| ZNF816 | 40 |
| ZNF680 | 41 |
| ZNF41 | 42 |
| ZNF189 | 43 |
| ZNF528 | 44 |
| ZNF543 | 45 |
| ZNF554 | 46 |
| ZNF140 | 47 |
| ZNF610 | 48 |
| ZNF264 | 49 |
| ZNF350 | 50 |
| ZNF8 | 51 |
| ZNF582 | 52 |
| ZNF30 | 53 |
| ZNF324 | 54 |
| ZNF98 | 55 |
| ZNF669 | 56 |
| ZNF677 | 57 |
| ZNF596 | 58 |
| ZNF214 | 59 |
| ZNF37A | 60 |
| ZNF34 | 61 |
| ZNF250 | 62 |
| ZNF547 | 63 |
| ZNF273 | 64 |
| ZNF354A | 65 |
| ZFP82 | 66 |
| ZNF224 | 67 |
| ZNF33A | 68 |
| ZNF45 | 69 |
| ZNF175 | 70 |
| ZNF595 | 71 |
| ZNF184 | 72 |
| ZNF419 | 73 |
| ZFP28-1 | 74 |
| ZFP28-2 | 75 |
| ZNF18 | 76 |
| ZNF213 | 77 |
| ZNF394 | 78 |
| ZFP1 | 79 |
| ZFP14 | 80 |
| ZNF416 | 81 |
| ZNF557 | 82 |
| ZNF566 | 83 |
| ZNF729 | 84 |
| ZIM2 | 85 |
| ZNF254 | 86 |
| ZNF764 | 87 |
| ZNF785 | 88 |
| ZNF10 (KOX1) | 89 |
| CBX5 (chromoshadow domain) | 90 |
| RYBP (YAF2_RYBP | 91 |
| component of PRC1) | |
| YAF2 (YAF2_RYBP | 92 |
| component of PRC1) | |
| MGA (component of PRC1.6) | 93 |
| CBX1 (chromoshadow) | 94 |
| SCMH1 (SAM_1/SPM) | 95 |
| MPP8 (Chromodomain) | 96 |
| SUMO3 (Rad60-SLD) | 97 |
| HERC2 (Cyt-b5) | 98 |
| BIN1 (SH3_9) | 99 |
| PCGF2 (RING finger protein | 100 |
| domain) | |
| TOX (HMG box) | 101 |
| FOXA1 (HNF3A C-terminal | 102 |
| domain) | |
| FOXA2 (HNF3B C-terminal | 103 |
| domain) | |
| IRF2BP1 (IRF-2BP1_2 N- | 104 |
| terminal domain) | |
| IRF2BP2 (IRF-2BP1_2 N- | 105 |
| terminal domain) | |
| IRF2BPL IRF-2BP1_2 N- | 106 |
| terminal domain | |
| HOXA13 (homeodomain) | 107 |
| HOXB13 (homeodomain) | 108 |
| HOXC13 (homeodomain) | 109 |
| HOXA11 (homeodomain) | 110 |
| HOXC11 (homeodomain) | 111 |
| HOXC10 (homeodomain) | 112 |
| HOXA10 (homeodomain) | 113 |
| HOXB9 (homeodomain) | 114 |
| HOXA9 (homeodomain) | 115 |
| ZFP28_HUMAN | 116 |
| ZN334_HUMAN | 117 |
| ZN568_HUMAN | 118 |
| ZN37A_HUMAN | 119 |
| ZN181_HUMAN | 120 |
| ZN510_HUMAN | 121 |
| ZN862_HUMAN | 122 |
| ZN140_HUMAN | 123 |
| ZN208_HUMAN | 124 |
| ZN248_HUMAN | 125 |
| ZN571_HUMAN | 126 |
| ZN699_HUMAN | 127 |
| ZN726_HUMAN | 128 |
| ZIK1_HUMAN | 129 |
| ZNF2_HUMAN | 130 |
| Z705F_HUMAN | 131 |
| ZNF14_HUMAN | 132 |
| ZN471_HUMAN | 133 |
| ZN624_HUMAN | 134 |
| ZNF84_HUMAN | 135 |
| ZNF7_HUMAN | 136 |
| ZN891_HUMAN | 137 |
| ZN337_HUMAN | 138 |
| Z705G_HUMAN | 139 |
| ZN529_HUMAN | 140 |
| ZN729_HUMAN | 141 |
| ZN419_HUMAN | 142 |
| Z705A_HUMAN | 143 |
| ZNF45_HUMAN | 144 |
| ZN302_HUMAN | 145 |
| ZN486_HUMAN | 146 |
| ZN621_HUMAN | 147 |
| ZN688_HUMAN | 148 |
| ZN33A_HUMAN | 149 |
| ZN554_HUMAN | 150 |
| ZN878_HUMAN | 151 |
| ZN772_HUMAN | 152 |
| ZN224_HUMAN | 153 |
| ZN184_HUMAN | 154 |
| ZN544_HUMAN | 155 |
| ZNF57_HUMAN | 156 |
| ZN283_HUMAN | 157 |
| ZN549_HUMAN | 158 |
| ZN211_HUMAN | 159 |
| ZN615_HUMAN | 160 |
| ZN253_HUMAN | 161 |
| ZN226_HUMAN | 162 |
| ZN730_HUMAN | 163 |
| Z585A_HUMAN | 164 |
| ZN732_HUMAN | 165 |
| ZN681_HUMAN | 166 |
| ZN667_HUMAN | 167 |
| ZN649_HUMAN | 168 |
| ZN470_HUMAN | 169 |
| ZN484_HUMAN | 170 |
| ZN431_HUMAN | 171 |
| ZN382_HUMAN | 172 |
| ZN254_HUMAN | 173 |
| ZN124_HUMAN | 174 |
| ZN607_HUMAN | 175 |
| ZN317_HUMAN | 176 |
| ZN620_HUMAN | 177 |
| ZN141_HUMAN | 178 |
| ZN584_HUMAN | 179 |
| ZN540_HUMAN | 180 |
| ZN75D_HUMAN | 181 |
| ZN555_HUMAN | 182 |
| ZN658_HUMAN | 183 |
| ZN684_HUMAN | 184 |
| RBAK_HUMAN | 185 |
| ZN829_HUMAN | 186 |
| ZN582_HUMAN | 187 |
| ZN112_HUMAN | 188 |
| ZN716_HUMAN | 189 |
| HKR1_HUMAN | 190 |
| ZN350_HUMAN | 191 |
| ZN480_HUMAN | 192 |
| ZN416_HUMAN | 193 |
| ZNF92_HUMAN | 194 |
| ZN100_HUMAN | 195 |
| ZN736_HUMAN | 196 |
| ZNF74_HUMAN | 197 |
| CBX1_HUMAN | 198 |
| ZN443_HUMAN | 199 |
| ZN195_HUMAN | 200 |
| ZN530_HUMAN | 201 |
| ZN782_HUMAN | 202 |
| ZN791_HUMAN | 203 |
| ZN331_HUMAN | 204 |
| Z354C_HUMAN | 205 |
| ZN157_HUMAN | 206 |
| ZN727_HUMAN | 207 |
| ZN550_HUMAN | 208 |
| ZN793_HUMAN | 209 |
| ZN235_HUMAN | 210 |
| ZNF8_HUMAN | 211 |
| ZN724_HUMAN | 212 |
| ZN573_HUMAN | 213 |
| ZN577_HUMAN | 214 |
| ZN789_HUMAN | 215 |
| ZN718_HUMAN | 216 |
| ZN300_HUMAN | 217 |
| ZN383_HUMAN | 218 |
| ZN429_HUMAN | 219 |
| ZN677_HUMAN | 220 |
| ZN850_HUMAN | 221 |
| ZN454_HUMAN | 222 |
| ZN257_HUMAN | 223 |
| ZN264_HUMAN | 224 |
| ZFP82_HUMAN | 225 |
| ZFP14_HUMAN | 226 |
| ZN485_HUMAN | 227 |
| ZN737_HUMAN | 228 |
| ZNF44_HUMAN | 229 |
| ZN596_HUMAN | 230 |
| ZN565_HUMAN | 231 |
| ZN543_HUMAN | 232 |
| ZFP69_HUMAN | 233 |
| SUMO1_HUMAN | 234 |
| ZNF12_HUMAN | 235 |
| ZN169_HUMAN | 236 |
| ZN433_HUMAN | 237 |
| SUMO3_HUMAN | 238 |
| ZNF98_HUMAN | 239 |
| ZN175_HUMAN | 240 |
| ZN347_HUMAN | 241 |
| ZNF25_HUMAN | 242 |
| ZN519_HUMAN | 243 |
| Z585B_HUMAN | 244 |
| ZIM3_HUMAN | 245 |
| ZN517_HUMAN | 246 |
| ZN846_HUMAN | 247 |
| ZN230_HUMAN | 248 |
| ZNF66_HUMAN | 249 |
| ZFP1_HUMAN | 250 |
| ZN713_HUMAN | 251 |
| ZN816_HUMAN | 252 |
| ZN426_HUMAN | 253 |
| ZN674_HUMAN | 254 |
| ZN627_HUMAN | 255 |
| ZNF20_HUMAN | 256 |
| Z587B_HUMAN | 257 |
| ZN316_HUMAN | 258 |
| ZN233_HUMAN | 259 |
| ZN611_HUMAN | 260 |
| ZN556_HUMAN | 261 |
| ZN234_HUMAN | 262 |
| ZN560_HUMAN | 263 |
| ZNF77_HUMAN | 264 |
| ZN682_HUMAN | 265 |
| ZN614_HUMAN | 266 |
| ZN785_HUMAN | 267 |
| ZN445_HUMAN | 268 |
| ZFP30_HUMAN | 269 |
| ZN225_HUMAN | 270 |
| ZN551_HUMAN | 271 |
| ZN610_HUMAN | 272 |
| ZN528_HUMAN | 273 |
| ZN284_HUMAN | 274 |
| ZN418_HUMAN | 275 |
| MPP8_HUMAN | 276 |
| ZN490_HUMAN | 277 |
| ZN805_HUMAN | 278 |
| Z780B_HUMAN | 279 |
| ZN763_HUMAN | 280 |
| ZN285_HUMAN | 281 |
| ZNF85_HUMAN | 282 |
| ZN223_HUMAN | 283 |
| ZNF90_HUMAN | 284 |
| ZN557_HUMAN | 285 |
| ZN425_HUMAN | 286 |
| ZN229_HUMAN | 287 |
| ZN606_HUMAN | 288 |
| ZN155_HUMAN | 289 |
| ZN222_HUMAN | 290 |
| ZN442_HUMAN | 291 |
| ZNF91_HUMAN | 292 |
| ZN135_HUMAN | 293 |
| ZN778_HUMAN | 294 |
| RYBP_HUMAN | 295 |
| ZN534_HUMAN | 296 |
| ZN586_HUMAN | 297 |
| ZN567_HUMAN | 298 |
| ZN440_HUMAN | 299 |
| ZN583_HUMAN | 300 |
| ZN441_HUMAN | 301 |
| ZNF43_HUMAN | 302 |
| CBX5_HUMAN | 303 |
| ZN589_HUMAN | 304 |
| ZNF10_HUMAN | 305 |
| ZN563_HUMAN | 306 |
| ZN561_HUMAN | 307 |
| ZN136_HUMAN | 308 |
| ZN630_HUMAN | 309 |
| ZN527_HUMAN | 310 |
| ZN333_HUMAN | 311 |
| Z324B_HUMAN | 312 |
| ZN786_HUMAN | 313 |
| ZN709_HUMAN | 314 |
| ZN792_HUMAN | 315 |
| ZN599_HUMAN | 316 |
| ZN613_HUMAN | 317 |
| ZF69B_HUMAN | 318 |
| ZN799_HUMAN | 319 |
| ZN569_HUMAN | 320 |
| ZN564_HUMAN | 321 |
| ZN546_HUMAN | 322 |
| ZFP92_HUMAN | 323 |
| YAF2_HUMAN | 324 |
| ZN723_HUMAN | 325 |
| ZNF34_HUMAN | 326 |
| ZN439_HUMAN | 327 |
| ZFP57_HUMAN | 328 |
| ZNF19_HUMAN | 329 |
| ZN404_HUMAN | 330 |
| ZN274_HUMAN | 331 |
| CBX3_HUMAN | 332 |
| ZNF30_HUMAN | 333 |
| ZN250_HUMAN | 334 |
| ZN570_HUMAN | 335 |
| ZN675_HUMAN | 336 |
| ZN695_HUMAN | 337 |
| ZN548_HUMAN | 338 |
| ZN132_HUMAN | 339 |
| ZN738_HUMAN | 340 |
| ZN420_HUMAN | 341 |
| ZN626_HUMAN | 342 |
| ZN559_HUMAN | 343 |
| ZN460_HUMAN | 344 |
| ZN268_HUMAN | 345 |
| ZN304_HUMAN | 346 |
| ZIM2_HUMAN | 347 |
| ZN605_HUMAN | 348 |
| ZN844_HUMAN | 349 |
| SUMO5_HUMAN | 350 |
| ZN101_HUMAN | 351 |
| ZN783_HUMAN | 352 |
| ZN417_HUMAN | 353 |
| ZN182_HUMAN | 354 |
| ZN823_HUMAN | 355 |
| ZN177_HUMAN | 356 |
| ZN197_HUMAN | 357 |
| ZN717_HUMAN | 358 |
| ZN669_HUMAN | 359 |
| ZN256_HUMAN | 360 |
| ZN251_HUMAN | 361 |
| CBX4_HUMAN | 362 |
| PCGF2_HUMAN | 363 |
| CDY2_HUMAN | 364 |
| CDYL2_HUMAN | 365 |
| HERC2_HUMAN | 366 |
| ZN562_HUMAN | 367 |
| ZN461_HUMAN | 368 |
| Z324A_HUMAN | 369 |
| ZN766_HUMAN | 370 |
| ID2_HUMAN | 371 |
| TOX_HUMAN | 372 |
| ZN274_HUMAN | 373 |
| SCMH1_HUMAN | 374 |
| ZN214_HUMAN | 375 |
| CBX7_HUMAN | 376 |
| ID1_HUMAN | 377 |
| CREM_HUMAN | 378 |
| SCX_HUMAN | 379 |
| ASCL1_HUMAN | 380 |
| ZN764_HUMAN | 381 |
| SCML2_HUMAN | 382 |
| TWST1_HUMAN | 383 |
| CREB1_HUMAN | 384 |
| TERF1_HUMAN | 385 |
| ID3_HUMAN | 386 |
| CBX8_HUMAN | 387 |
| CBX4_HUMAN | 388 |
| GSX1_HUMAN | 389 |
| NKX22_HUMAN | 390 |
| ATF1_HUMAN | 391 |
| TWST2_HUMAN | 392 |
| ZNF17_HUMAN | 393 |
| TOX3_HUMAN | 394 |
| TOX4_HUMAN | 395 |
| ZMYM3_HUMAN | 396 |
| I2BP1_HUMAN | 397 |
| RHXF1_HUMAN | 398 |
| SSX2_HUMAN | 399 |
| I2BPL_HUMAN | 400 |
| ZN680_HUMAN | 401 |
| CBX1_HUMAN | 402 |
| TRI68_HUMAN | 403 |
| HXA13_HUMAN | 404 |
| PHC3_HUMAN | 405 |
| TCF24_HUMAN | 406 |
| CBX3_HUMAN | 407 |
| HXB13_HUMAN | 408 |
| HEY1_HUMAN | 409 |
| PHC2_HUMAN | 410 |
| ZNF81_HUMAN | 411 |
| FIGLA_HUMAN | 412 |
| SAM11_HUMAN | 413 |
| KMT2B_HUMAN | 414 |
| HEY2_HUMAN | 415 |
| JDP2_HUMAN | 416 |
| HXC13_HUMAN | 417 |
| ASCL4_HUMAN | 418 |
| HHEX_HUMAN | 419 |
| HERC2_HUMAN | 420 |
| GSX2_HUMAN | 421 |
| BIN1_HUMAN | 422 |
| ETV7_HUMAN | 423 |
| ASCL3_HUMAN | 424 |
| PHC1_HUMAN | 425 |
| OTP_HUMAN | 426 |
| I2BP2_HUMAN | 427 |
| VGLL2_HUMAN | 428 |
| HXA11_HUMAN | 429 |
| PDLI4_HUMAN | 430 |
| ASCL2_HUMAN | 431 |
| CDX4_HUMAN | 432 |
| ZN860_HUMAN | 433 |
| LMBL4_HUMAN | 434 |
| PDIP3_HUMAN | 435 |
| NKX25_HUMAN | 436 |
| CEBPB_HUMAN | 437 |
| ISL1_HUMAN | 438 |
| CDX2_HUMAN | 439 |
| PROP1_HUMAN | 440 |
| SIN3B_HUMAN | 441 |
| SMBT1_HUMAN | 442 |
| HXC11_HUMAN | 443 |
| HXC10_HUMAN | 444 |
| PRS6A_HUMAN | 445 |
| VSX1_HUMAN | 446 |
| NKX23_HUMAN | 447 |
| MTG16_HUMAN | 448 |
| HMX3_HUMAN | 449 |
| HMX1_HUMAN | 450 |
| KIF22_HUMAN | 451 |
| CSTF2_HUMAN | 452 |
| CEBPE_HUMAN | 453 |
| DLX2_HUMAN | 454 |
| ZMYM3_HUMAN | 455 |
| PPARG_HUMAN | 456 |
| PRIC1_HUMAN | 457 |
| UNC4_HUMAN | 458 |
| BARX2_HUMAN | 459 |
| ALX3_HUMAN | 460 |
| TCF15_HUMAN | 461 |
| TERA_HUMAN | 462 |
| VSX2_HUMAN | 463 |
| HXD12_HUMAN | 464 |
| CDX1_HUMAN | 465 |
| TCF23_HUMAN | 466 |
| ALX1_HUMAN | 467 |
| HXA10_HUMAN | 468 |
| RX_HUMAN | 469 |
| CXXC5_HUMAN | 470 |
| SCML1_HUMAN | 471 |
| NFIL3_HUMAN | 472 |
| DLX6_HUMAN | 473 |
| MTG8_HUMAN | 474 |
| CBX8_HUMAN | 475 |
| CEBPD_HUMAN | 476 |
| SEC13_HUMAN | 477 |
| FIP1_HUMAN | 478 |
| ALX4_HUMAN | 479 |
| LHX3_HUMAN | 480 |
| PRIC2_HUMAN | 481 |
| MAGI3_HUMAN | 482 |
| NELL1_HUMAN | 483 |
| PRRX1_HUMAN | 484 |
| MTG8R_HUMAN | 485 |
| RAX2_HUMAN | 486 |
| DLX3_HUMAN | 487 |
| DLX1_HUMAN | 488 |
| NKX26_HUMAN | 489 |
| NAB1_HUMAN | 490 |
| SAMD7_HUMAN | 491 |
| PITX3_HUMAN | 492 |
| WDR5_HUMAN | 493 |
| MEOX2_HUMAN | 494 |
| NAB2_HUMAN | 495 |
| DHX8_HUMAN | 496 |
| FOXA2_HUMAN | 497 |
| CBX6_HUMAN | 498 |
| EMX2_HUMAN | 499 |
| CPSF6_HUMAN | 500 |
| HXC12_HUMAN | 501 |
| KDM4B_HUMAN | 502 |
| LMBL3_HUMAN | 503 |
| PHX2A_HUMAN | 504 |
| EMX1_HUMAN | 505 |
| NC2B_HUMAN | 506 |
| DLX4_HUMAN | 507 |
| SRY_HUMAN | 508 |
| ZN777_HUMAN | 509 |
| NELL1_HUMAN | 510 |
| ZN398_HUMAN | 511 |
| GATA3_HUMAN | 512 |
| BSH_HUMAN | 513 |
| SF3B4_HUMAN | 514 |
| TEAD1_HUMAN | 515 |
| TEAD3_HUMAN | 516 |
| RGAP1_HUMAN | 517 |
| PHF1_HUMAN | 518 |
| FOXA1_HUMAN | 519 |
| GATA2_HUMAN | 520 |
| FOXO3_HUMAN | 521 |
| ZN212_HUMAN | 522 |
| IRX4_HUMAN | 523 |
| ZBED6_HUMAN | 524 |
| LHX4_HUMAN | 525 |
| SIN3A_HUMAN | 526 |
| RBBP7_HUMAN | 527 |
| NKX61_HUMAN | 528 |
| TRI68_HUMAN | 529 |
| R51A1_HUMAN | 530 |
| MB3L1_HUMAN | 531 |
| DLX5_HUMAN | 532 |
| NOTC1_HUMAN | 533 |
| TERF2_HUMAN | 534 |
| ZN282_HUMAN | 535 |
| RGS12_HUMAN | 536 |
| ZN840_HUMAN | 537 |
| SPI2B_HUMAN_1 | 538 |
| PAX7_HUMAN | 539 |
| NKX62_HUMAN | 540 |
| ASXL2_HUMAN | 541 |
| FOXO1_HUMAN | 542 |
| GATA3_HUMAN | 543 |
| GATA1_HUMAN | 544 |
| ZMYM5_HUMAN | 545 |
| ZN783_HUMAN | 546 |
| SPI2B_HUMAN_2 | 547 |
| LRP1_HUMAN | 548 |
| MIXL1_HUMAN | 549 |
| SGT1_HUMAN | 550 |
| LMCD1_HUMAN | 551 |
| CEBPA_HUMAN | 552 |
| GATA2_HUMAN | 553 |
| SOX14_HUMAN | 554 |
| WTIP_HUMAN | 555 |
| PRP19_HUMAN | 556 |
| CBX6_HUMAN | 557 |
| NKX11_HUMAN | 558 |
| RBBP4_HUMAN | 559 |
| DMRT2_HUMAN | 560 |
| SMCA2_HUMAN | 561 |
| ZNF10_HUMAN | 562 |
| EED_HUMAN | 563 |
| RCOR1_HUMAN | 564 |

A functional analog of any one of the above-listed proteins, i.e., a molecule having the same or substantially the same biological function (e.g., retaining 70% or more, 80% or more, 90% or more, 95% or more, or 98% or more) of the protein's transcription factor function) is encompassed by the present disclosure. For example, the functional analog may be an isoform or a variant of the above-listed protein, e.g., containing a portion of the above protein with or without additional amino acid residues and/or containing mutations relative to the above protein. In some embodiments, the functional analog has a sequence identity that is at least 75, 80, 85, 90, 95, 98, or 99% to one of the sequences listed in Table 13. Homologs, orthologs, and mutants of the above-listed proteins are also contemplated.

In certain embodiments, an epigenetic editor described herein comprises a KRAB domain derived from KOX1, ZIM3, ZFP28, or ZN627, and/or an effector domain derived from KAP1, MECP2, HP1a, HP1b, CBX8, CDYL2, TOX, TOX3, TOX4, EED, EZH2, RBBP4, RCOR1, or SCML2, optionally wherein the parental protein is a human protein. In particular embodiments, an epigenetic editor described herein comprises a domain derived from KOX1, ZIM3, ZFP28, and/or ZN627, optionally wherein the parental protein is a human protein. In certain embodiments, the epigenetic editor may comprise a KRAB domain derived from KOX1 (ZNF10), e.g., a human KOX1. In certain embodiments, the epigenetic editor may comprise a KRAB domain derived from ZIM3 (ZNF657 or ZNF264), e.g., a human ZIM3. In certain embodiments, the epigenetic editor may comprise a KRAB domain derived from ZFP28, e.g., a human ZFP28. In certain embodiments, the epigenetic editor may comprise a KRAB domain derived from ZN627, e.g., a human ZN627. In certain embodiments, an epigenetic editor described herein may comprise a CDYL2, e.g., a human CDYL2, and/or a TOX domain (e.g., a human TOX domain) in combination with a KOX1 KRAB domain (e.g., a human KOX1 KRAB domain).

In certain embodiments, an epigenetic effector described herein comprises a repressor domain derived from KOX1/ZNF10 (SEQ ID NO: 89). For example, the repressor domain may comprise the sequence of SEQ ID NO: 89, or a sequence at least 75%, 80%, 85%, 90%, 91%, 92%, 93%, 94%, 95%, 96%, 97%, 98%, or 99% identical to SEQ ID NO: 89.

In certain embodiments, an epigenetic effector described herein comprises a repressor domain derived from KOX1/ZNF10, as shown in Table 14 below:

TABLE 14
Exemplary Effector Domains Derived from KOX1/ZNF10

| Protein | Protein Sequence |
|---|---|
| KOX1/ZNF10 KRAB 1 | SEQ ID NO: 565 |
| KOX1/ZNF10 KRAB 2 | SEQ ID NO: 566 |
| KOX1/ZNF10 KRAB 3 | SEQ ID NO: 567 |
| KOX1/ZNF10 (aa 11-72) | SEQ ID NO: 568 |
| KOX1/ZNF10 (aa 11-108) | SEQ ID NO: 569 |
| KOX1/ZNF10 variant | SEQ ID NO: 570 |
| KOX1 KRAB-ZIM3 chimera | SEQ ID NO: 571 |
| ZIM3-KOX1 KRAB chimera | SEQ ID NO: 572 |

In particular embodiments, the repressor domain may comprise the amino acid sequence of SEQ ID NO: 565, or a sequence at least 75%, 80%, 85%, 90%, 91%, 92%, 93%, 94%, 95%, 96%, 97%, 98%, or 99% identical to SEQ ID NO: 565.

In particular embodiments, the repressor domain may comprise the amino acid sequence of SEQ ID NO: 566, or a sequence at least 75%, 80%, 85%, 90%, 91%, 92%, 93%, 94%, 95%, 96%, 97%, 98%, or 99% identical to SEQ ID NO: 566.

In particular embodiments, the repressor domain may comprise the amino acid sequence of SEQ ID NO: 567, or a sequence at least 75%, 80%, 85%, 90%, 91%, 92%, 93%, 94%, 95%, 96%, 97%, 98%, or 99% identical to SEQ ID NO: 567.

In particular embodiments, the repressor domain may comprise the amino acid sequence of SEQ ID NO: 568, or a sequence at least 75%, 80%, 85%, 90%, 91%, 92%, 93%, 94%, 95%, 96%, 97%, 98%, or 99% identical to SEQ ID NO: 568.

In particular embodiments, the repressor domain may comprise the amino acid sequence of SEQ ID NO: 569, or a sequence at least 75%, 80%, 85%, 90%, 91%, 92%, 93%, 94%, 95%, 96%, 97%, 98%, or 99% identical to SEQ ID NO: 569.

In particular embodiments, the repressor domain may comprise the amino acid sequence of SEQ ID NO: 570, or a sequence at least 75%, 80%, 85%, 90%, 91%, 92%, 93%, 94%, 95%, 96%, 97%, 98%, or 99% identical to SEQ ID NO: 570.

In particular embodiments, the repressor domain may comprise the amino acid sequence of SEQ ID NO: 571, or a sequence at least 75%, 80%, 85%, 90%, 91%, 92%, 93%, 94%, 95%, 96%, 97%, 98%, or 99% identical to SEQ ID NO: 571.

In particular embodiments, the repressor domain may comprise the amino acid sequence of SEQ ID NO: 572, or a sequence at least 75%, 80%, 85%, 90%, 91%, 92%, 93%, 94%, 95%, 96%, 97%, 98%, or 99% identical to SEQ ID NO: 572.

### B. DNA Methyltransferases

In some embodiments, an effector domain of an epigenetic editor described herein alters target gene expression through DNA modification, such as methylation. Highly methylated areas of DNA tend to be less transcriptionally active than less methylated areas. DNA methylation occurs primarily at CpG sites (shorthand for “C-phosphate-G-” or “cytosine-phosphate-guanine” sites). Many mammalian genes have promoter regions near or including CpG islands (nucleic acid regions with a high frequency of CpG dinucleotides).

An effector domain described herein may be, e.g., a DNA methyltransferase (DNMT) or a catalytic domain thereof, or may be capable of recruiting a DNA methyltransferase. DNMTs encompass enzymes that catalyze the transfer of a methyl group to a DNA nucleotide, such as canonical cytosine-5 DNMTs that catalyze the addition of methyl groups to genomic DNA (e.g., DNMT1, DNMT3A, DNMT3B, and DNMT3C). This term also encompasses non-canonical family members that do not catalyze methylation themselves but that recruit (including activate) catalytically active DNMTs; a non-limiting example of such a DNMT is DNMT3L. See, e.g., Lyko, Nat Review (2018) 19:81-92. Unless otherwise indicated, a DNMT domain may refer to a polypeptide domain derived from a catalytically active DNMT (e.g., DNMT1, DNMT3A, and DNMT3B) or from a catalytically inactive DNMT (e.g., DNMT3L). A DNMT may repress expression of the target gene through the recruitment of repressive regulatory proteins. In some embodiments, the methylation is at a CG (or CpG) dinucleotide sequence. In some embodiments, the methylation is at a CHG or CHH sequence, where H is any one of A, T, or C.

In some embodiments, a DNMT described herein can be an animal DNMT (e.g., a mammalian DNMT), a plant DNMT, a fungal DNMT, or a bacterial DNMT. A bacterial DNMT can be obtained from a bacterial species (e.g., a coccus bacterium, bacillus bacterium, spiral bacterium, or an intracellular, gram-positive, or gram-negative bacterium. In certain embodiments, the bacterial species is Mycoplasmatales bacterium, Mycoplasma marinum, or Spiroplasma chinense. In certain embodiments, the bacterial species is not M. penetrans, S. monbiae, H. parainfluenzae, A. luteus, H. aegyptius, H. haemolyticus, Moraxella, E. coli, T. aquaticus, C. crescentus, or C. difficile. In certain embodiments, an epigenetic editor described herein comprises a DNMT domain comprising SEQ ID NO: 601, or a sequence at least 75%, 80%, 85%, 90%, 91%, 92%, 93%, 94%, 95%, 96%, 97%, 98%, or 99% identical to SEQ ID NO: 601. In certain embodiments, an epigenetic editor described herein comprises a DNMT domain comprising SEQ ID NO: 602, or a sequence at least 75%, 80%, 85%, 90%, 91%, 92%, 93%, 94%, 95%, 96%, 97%, 98%, or 99% identical to SEQ ID NO: 602. In certain embodiments, an epigenetic editor described herein comprises a DNMT domain comprising SEQ ID NO: 603, or a sequence at least 75%, 80%, 85%, 90%, 91%, 92%, 93%, 94%, 95%, 96%, 97%, 98%, or 99% identical to SEQ ID NO: 603.

In certain embodiments, DNMTs in the epigenetic editors described herein may include, e.g., DNMT1, DNMT3A, DNMT3B, and/or DNMT3C. In some embodiments, the DNMT is a mammalian (e.g., human or murine) DNMT. In particular embodiments, the DNMT is DNMT3A (e.g., human DNMT3A). In certain embodiments, an epigenetic editor described herein comprises a DNMT3A domain comprising SEQ ID NO: 574, or a sequence at least 75%, 80%, 85%, 90%, 91%, 92%, 93%, 94%, 95%, 96%, 97%, 98%, or 99% identical to SEQ ID NO: 574. In certain embodiments, an epigenetic editor described herein comprises a DNMT3A domain comprising SEQ ID NO: 575, or a sequence at least 75%, 80%, 85%, 90%, 91%, 92%, 93%, 94%, 95%, 96%, 97%, 98%, or 99% identical to SEQ ID NO: 575. In some embodiments, the DNMT3A domain may have, e.g., a mutation at position H739 (such as H739A or H739E), R771 (such as R771L) and/or R836 (such as R836A or R836Q), or any combination thereof (numbering according to SEQ ID NO: 574).

In some embodiments, an effector domain described herein may be a DNMT-like domain. As used herein a “DNMT-like domain” is a regulatory factor of DNMT that may activate or recruit other DNMT domains, but does not itself possess methylation activity. In some embodiments, the DNMT-like domain is a mammalian (e.g., human or mouse) DNMT-like domain. In certain embodiments, the DNMT-like domain is DNMT3L, which may be, for example, human DNMT3L or mouse DNMT3L. In certain embodiments, an epigenetic editor described herein comprises a DNMT3L domain comprising SEQ ID NO: 578, or a sequence at least 75%, 80%, 85%, 90%, 91%, 92%, 93%, 94%, 95%, 96%, 97%, 98%, or 99% identical to SEQ ID NO: 578. In certain embodiments, an epigenetic editor herein comprises a DNMT3L domain comprising SEQ ID NO: 579, or a sequence at least 75%, 80%, 85%, 90%, 91%, 92%, 93%, 94%, 95%, 96%, 97%, 98%, or 99% identical to SEQ ID NO: 579. In certain embodiments, an epigenetic editor described herein comprises a DNMT3L domain comprising SEQ ID NO: 580, or a sequence at least 75%, 80%, 85%, 90%, 91%, 92%, 93%, 94%, 95%, 96%, 97%, 98%, or 99% identical to SEQ ID NO: 580. In certain embodiments, an epigenetic editor described herein comprises a DNMT3L domain comprising SEQ ID NO: 581, or a sequence at least 75%, 80%, 85%, 90%, 91%, 92%, 93%, 94%, 95%, 96%, 97%, 98%, or 99% identical to SEQ ID NO: 581. In some embodiments, the DNMT3L domain may have, e.g., a mutation corresponding to that at position D226 (such as D226V), Q268 (such as Q268K), or both (numbering according to SEQ ID NO: 578).

In certain embodiments, an epigenetic editor herein may comprise comprising both DNMT and DNMT-like effector domains. For example, the epigenetic editor may comprise a DNMT3A-3L domain, wherein DNMT3A and DNMT3L may be covalently linked. In other embodiments, an epigenetic editor described herein may comprise an effector domain that comprises only a DNMT3A domain (e.g., human DNMT3A), or only a DNMT-like domain (e.g., DNMT3L, which may be human or mouse DNMT3L).

Table 15 below provides exemplary DNMTs that may be part of an epigenetic effector described herein, or from which an effector domain of an epigenetic editor described herein may be derived.

TABLE 15
Exemplary DNMT Sequences

| Protein Name | Species | Target | Protein Sequence |
|---|---|---|---|
| DNMT1 | Human | 5mC | SEQ ID NO: 573 |
| DNMT3A (h3A) | Human | 5mC | SEQ ID NO: 574 |
| DNMT3A | Human | 5mC | SEQ ID NO: 575 |
| (catalytic domain) | | | |
| (h3As) | | | |
| DNMT3B | Human | 5mC | SEQ ID NO: 576 |
| DNMT3C | Mouse | 5mC | SEQ ID NO: 577 |
| DNMT3L (h3L) | Human | 5mC | SEQ ID NO: 578 |
| DNMT3L | Human | 5mC | SEQ ID NO: 579 |
| (catalytic domain) | | | |
| (h3Ls) | | | |
| DNMT3L (m3L) | Mouse | 5mC | SEQ ID NO: 580 |
| DNMT3L | Mouse | 5mC | SEQ ID NO: 581 |
| (catalytic domain) | | | |
| (m3Ls) | | | |
| DNMT3L | Ailuropoda melanoleuca | 5mC | SEQ ID NO: 582 |
| DNMT3L | Ailuropoda melanoleuca | 5mC | SEQ ID NO: 583 |
| (catalytic domain) | | | |
| DNMT3L | Carlito syrichta | 5mC | SEQ ID NO: 584 |
| DNMT3L | Carlito syrichta | 5mC | SEQ ID NO: 585 |
| (catalytic domain) | | | |
| DNMT3L | Meriones unguiculatus | 5mC | SEQ ID NO: 586 |
| DNMT3L | Meriones unguiculatus | 5mC | SEQ ID NO: 587 |
| (catalytic domain) | | | |
| DNMT3L | Ochotona princeps | 5mC | SEQ ID NO: 588 |
| DNMT3L | Ochotona princeps | 5mC | SEQ ID NO: 589 |
| (catalytic domain) | | | |
| DNMT3L | Neosciurus carolinensis | 5mC | SEQ ID NO: 590 |
| DNMT3L | Neosciurus carolinensis | 5mC | SEQ ID NO: 591 |
| (catalytic domain) | | | |
| DNMT3L | Bison bison | 5mC | SEQ ID NO: 592 |
| DNMT3L | Bison bison | 5mC | SEQ ID NO: 593 |
| (catalytic domain) | | | |
| DNMT3L | Equus przewalskii | 5mC | SEQ ID NO: 594 |
| DNMT3L | Equus przewalskii | 5mC | SEQ ID NO: 595 |
| (catalytic domain) | | | |
| DNMT3L | Mus caroli | 5mC | SEQ ID NO: 596 |
| DNMT3L | Mus caroli | 5mC | SEQ ID NO: 597 |
| (catalytic domain) | | | |
| DNMT3L | Pan troglodytes | 5mC | SEQ ID NO: 598 |
| DNMT3L | Pan troglodytes | 5mC | SEQ ID NO: 599 |
| (catalytic domain) | | | |
| TRDMT1 | Human | tRNA 5mC | SEQ ID NO: 600 |
| (DNMT2) | | | |
| DNA cytosine | Mycoplasmatales | 5mC | SEQ ID NO: 601 |
| methyltransferase | bacterium | | |
| DNA cytosine | Mycoplasma marinum | 5mC | SEQ ID NO: 602 |
| methyltransferase | | | |
| DNA (cytosine-5-)- | Spiroplasma chinense | 5mC | SEQ ID NO: 603 |
| methyltransferase | | | |
| M.MpeI | Mycoplasma penetrans | 5mC | SEQ ID NO: 604 |
| M.SssI | Spiroplasma monobiae | 5mC | SEQ ID NO: 605 |
| M.HpaII | Haemophilus | 5mC (CCGG) | SEQ ID NO: 606 |
| | parainfluenzae | | |
| M.AluI | Arthrobacter luteus | 5mC (AGCT) | SEQ ID NO: 607 |
| M.HaeIII | Haemophilus aegyptius | 5mC (GGCC) | SEQ ID NO: 608 |
| M.HhaI | Haemophilus | 5mC (GCGC) | SEQ ID NO: 609 |
| | haemolyticus | | |
| M.MspI | Moraxella | 5mC (CCGG) | SEQ ID NO: 610 |
| Masc1 | Ascobolus | 5mC | SEQ ID NO: 611 |
| MET1 | Arabidopsis | 5mC | SEQ ID NO: 612 |
| Masc2 | Ascobolus | 5mC | SEQ ID NO: 613 |
| Dim-2 | Neurospora | 5mC | SEQ ID NO: 614 |
| dDnmt2 | Drosophila | 5mC | SEQ ID NO: 615 |
| Pmt1 | S. pombe | 5mC | SEQ ID NO: 616 |
| DRM1 | Arabidopsis | 5mC | SEQ ID NO: 617 |
| DRM2 | Arabidopsis | 5mC | SEQ ID NO: 618 |
| CMT1 | Arabidopsis | 5mC | SEQ ID NO: 619 |
| CMT2 | Arabidopsis | 5mC | SEQ ID NO: 620 |
| CMT3 | Arabidopsis | 5mC | SEQ ID NO: 621 |
| Rid | Neurospora | 5mC | SEQ ID NO: 622 |
| hsdM gene | bacteria (E. coli, strain 12) | m6A | SEQ ID NO: 623 |
| hsdS gene | bacteria (E. coli, strain 12) | m6A | SEQ ID NO: 624 |
| M.TaqI | Bacteria (Thermus | m6A | SEQ ID NO: 625 |
| | aquaticus) | | |
| M.EcoDam | E. coli | m6A | SEQ ID NO: 626 |
| M.CcrMI | Caulobacter crescentus | m6A | SEQ ID NO: 627 |
| CamA | Clostridioides difficile | m6A | SEQ ID NO: 628 |

A functional analog of any one of the above-listed proteins, i.e., a molecule having the same or substantially the same biological function (e.g., retaining 70% or more, 80% or more, 90% or more, 95% or more, or 98% or more) of the protein's DNA methylation function or recruiting function is encompassed by the present disclosure. For example, the functional analog may be an isoform or a variant of the above-listed protein, e.g., containing a portion of the above protein with or without additional amino acid residues and/or containing mutations relative to the above protein. In some embodiments, the functional analog has a sequence identity that is at least 75, 80, 85, 90, 95, 98, or 99% to one of the sequences listed in Table 15. In some embodiments, the effector domain herein comprises only the functional domain (or functional analog thereof), e.g., the catalytic domain or recruiting domain, of an above-listed protein. In some embodiments, the effector domain herein comprises one or more epigenetic effector domains selected from Table 15, or functional homologs, orthologs, or variants thereof.

As used herein, a DNMT domain (e.g., a DNMT3A domain or a DNMT3L domain) refers to a protein domain that is identical to the parental protein (e.g., a human or murine DNMT3A or DNMT3L) or a functional analog thereof (e.g., having a functional fragment, such as a catalytic fragment or recruiting fragment, of the parental protein; and/or having mutations that improve the activity of the DNMT protein).

An epigenetic editor herein may effect methylation at, e.g., 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, or 1000 or more CpG dinucleotide sequences in the target gene or chromosome. The CpG dinucleotide sequences may be located within or near the target gene in CpG islands, or may be located in a region that is not a CpG island. A CpG island generally refers to a nucleic acid sequence or chromosome region that comprises a high frequency of CpG dinucleotides. For example, a CpG island may comprise at least 50% GC content. The CpG island may have a high observed-to-expected CpG ratio, for example, an observed-to-expected CpG ratio of at least 60%. As used herein, an observed-to-expected CpG ratio is determined by Number of CpG*(sequence length)/(Number of C*Number of G). In some embodiments, the CpG island has an observed-to-expected CpG ratio of at least 60%, 70%, 80%, 90% or more. A CpG island may be a sequence or region of, e.g., at least 200, 250, 300, 350, 400, 450, 500, 550, 600, 650, 700, 750, or 800 nucleotides. In some embodiments, only 1, or less than 2, 3, 4, 5, 6, 7, 8, 9, 10, 11, 12, 13, 14, 15, 16, 17, 18, 19, 20, 30, 40, or 50 CpG dinucleotides are methylated by the epigenetic editor.

In some embodiments, an epigenetic editor herein effects methylation at a hypomethylated nucleic acid sequence, i.e., a sequence that may lack methyl groups on the 5-methyl cytosine nucleotides (e.g., in CpG) as compared to a standard control. Hypomethylation may occur, for example, in aging cells or in cancer (e.g., early stages of neoplasia) relative to a younger cell or non-cancer cell, respectively.

In some embodiments, an epigenetic editor described herein induces methylation at a hypermethylated nucleic acid sequence.

In some embodiments, methylation may be introduced by the epigenetic editor at a site other than a CpG dinucleotide. For example, the target gene sequence may be methylated at the C nucleotide of CpA, CpT, or CpC sequences. In some embodiments, an epigenetic editor comprises a DNMT3A domain and effects methylation at CpG, CpA, CpT, CpC sequences, or any combination thereof. In some embodiments, an epigenetic editor comprises a DNMT3A domain that lacks a regulatory subdomain and only maintains a catalytic domain. In some embodiments, the epigenetic editor comprising a DNMT3A catalytic domain effects methylation exclusively at CpG sequences. In some embodiments, an epigenetic editor comprising a DNMT3A domain that comprises a mutation, e.g. a R836A or R836Q mutation (numbering according to SEQ ID NO: 574), has higher methylation activity at CpA, CpC, and/or CpT sequences as compared to an epigenetic editor comprising a wildtype DNMT3A domain.

### C. Histone Modifiers

In some embodiments, an effector domain of an epigenetic editor herein mediates histone modification. Histone modifications play a structural and biochemical role in gene transcription, such as by formation or disruption of the nucleosome structure that binds to the histone and prevents gene transcription. Histone modifications may include, for example, acetylation, deacetylation, methylation, phosphorylation, ubiquitination, SUMOylation and the like, e.g., at their N-terminal ends (“histone tails”). These modifications maintain or specifically convert chromatin structure, thereby controlling responses such as gene expression, DNA replication, DNA repair, and the like, which occur on chromosomal DNA. Post-translational modification of histones is an epigenetic regulatory mechanism and is considered essential for the genetic regulation of eukaryotic cells. Recent studies have revealed that chromatin remodeling factors such as SWI/SNF, RSC, NURF, NRD, and the like, which facilitate transcription factor access to DNA by modifying the nucleosome structure; histone acetyltransferases (HATs) that regulate the acetylation state of histones; and histone deacetylases (HDACs), act as important regulators.

In particular, the unstructured N-termini of histones may be modified by acetylation, deacetylation, methylation, ubiquitylation, phosphorylation, SUMOylation, ribosylation, citrullination O-GlcNAcylation, crotonylation, or any combination thereof. For example, histone acetyltransferases (HATs) utilize acetyl-CoA as a cofactor and catalyze the transfer of an acetyl group to the epsilon amino group of the lysine side chains. This neutralizes the lysine's positive charge and weakens the interactions between histones and DNA, thus opening the chromosomes for transcription factors to bind and initiate transcription. Acetylation of K14 and K9 lysines of histone H3 by histone acetyltransferase enzymes may be linked to transcriptional competence in humans. Lysine acetylation may directly or indirectly create binding sites for chromatin-modifying enzymes that regulate transcriptional activation. On the other hand, histone methylation of lysine 9 of histone H3 may be associated with heterochromatin, or transcriptionally silent chromatin.

In certain embodiments, an effector domain of an epigenetic editor described herein comprises a histone methyltransferase domain. The effector domain may comprise, for example, a DOT1L domain, a SET domain, a SUV39H1 domain, a G9a/EHMT2 protein domain, an EZH1 domain, an EZH2 domain, a SETDB1 domain, or any combination thereof. In particular embodiments, the effector domain comprises a histone-lysine-N-methyltransferase SETDB1 domain.

In some embodiments, the effector domain comprises a histone deacetylase protein domain. In certain embodiments, the effector domain comprises a HDAC family protein domain, for example, a HDAC1, HDAC3, HDAC5, HDAC7, or HDAC9 protein domain. In particular embodiments, the effector domain comprises a nucleosome remodeling and deacetylase complex (NURD), which removes acetyl groups from histones.

### D. Other Effector Domains

In some embodiments, the effector domain comprises a tripartite motif containing protein (TRIM28, TIF1-beta, or KAP1). In certain embodiments, the effector domain comprises one or more KAP1 proteins. A KAP1 protein in an epigenetic editor herein may form a complex with one or more other effector domains of the epigenetic editor or one or more proteins involved in modulation of gene expression in a cellular environment. For example, KAP1 may be recruited by a KRAB domain of a transcriptional repressor. A KAP1 protein domain may interact with or recruit one or more protein complexes that reduces or silences gene expression. In some embodiments, KAP1 interacts with or recruits a histone deacetylase protein, a histone-lysine methyltransferase protein, a chromatin remodeling protein, and/or a heterochromatin protein. For example, a KAP1 protein domain may interact with or recruit a heterochromatin protein 1 (HP1) protein, a SETDB1 protein, an HDAC protein, and/or a NuRD protein complex component. In some embodiments, a KAP1 protein domain interacts with or recruits a ZFP90 protein (e.g., isoform 2 of ZFP90), and/or a FOXP3 protein. An exemplary KAP1 amino acid sequence is shown in SEQ ID NO: 629.

In some embodiments, the effector domain comprises a protein domain that interacts with or is recruited by one or more DNA epigenetic marks. For example, the effector domain may comprise a methyl CpG binding protein 2 (MECP2) protein that interacts with methylated DNA nucleotides in the target gene (which may or may not be at a CpG island of the target gene). An MECP2 protein domain in an epigenetic editor described herein may induce condensed chromatin structure, thereby reducing or silencing expression of the target gene. In some embodiments, an MECP2 protein domain in an epigenetic editor described herein may interact with a histone deacetylase (e.g. HDAC), thereby repressing or silencing expression of the target gene. In some embodiments, an MECP2 protein domain in an epigenetic editor described herein may block access of a transcription factor or transcriptional activator to the target sequence, thereby repressing or silencing expression of the target gene. An exemplary MECP2 amino acid sequence is shown in SEQ ID NO: 630.

Also contemplated as effector domains for the epigenetic editors described herein are, e.g., a chromoshadow domain, a ubiquitin-2 like Rad60 SUMO-like (Rad60-SLD/SUMO) domain, a chromatin organization modifier domain (Chromo) domain, a Yaf2/RYBP C-terminal binding motif domain (YAF2_RYBP), a CBX family C-terminal motif domain (CBX7_C), a zinc finger C3HC4 type (RING finger) domain (ZF-C3HC4_2), a cytochrome b5 domain (Cyt-b5), a helix-loop-helix domain (HLH), a helix-hairpin-helix motif domain (e.g., HHH_3), a high mobility group box domain (HMG-box), a basic leucine zipper domain (e.g., bZIP_1 or bZIP_2), a Myb_DNA-binding domain, a homeodomain, a MYM-type zinc finger with FCS sequence domain (ZF-FCS), an interferon regulatory factor 2-binding protein zinc finger domain (IRF-2BP1_2), an SSX repressor domain (SSXRD), a B-box-type zinc finger domain (ZF-B_box), a CXXC zinc finger domain (ZF-CXXC), a regulator of chromosome condensation 1 domain (RCC1), an SRC homology 3 domain (SH3_9), a sterile alpha motif domain (SAM_1), a sterile alpha motif domain (SAM_2), a sterile alpha motif/Pointed domain (SAM_PNT), a Vestigial/Tondu family domain (Vg_Tdu), a LIM domain, an RNA recognition motif domain (RRM_1), a paired amphipathic helix domain (PAH), a proteasomal ATPase OB C-terminal domain (Prot_ATP_ID_OB), a nervy homology 2 domain (NHR2), a hinge domain of cleavage stimulation factor subunit 2 (CSTF2_hinge), a PPAR gamma N-terminal region domain (PPARgamma_N), a CDC48 N-terminal domain (CDC48_2), a WD40 repeat domain (WD40), a Fip1 motif domain (Fip1), a PDZ domain (PDZ_6), a Von Willebrand factor type C domain (VWC), a NAB conserved region 1 domain (NCD1), an S1 RNA-binding domain (S1), an HNF3 C-terminal domain (HNF_C), a Tudor domain (Tudor_2), a histone-like transcription factor (CBF/NF-Y) and archaeal histone domain (CBFD_NFYB_HMF), a zinc finger protein domain (DUF3669), an EGF-like domain (cEGF), a GATA zinc finger domain (GATA), a TEA/ATTS domain (TEA), a phorbol esters/diacylglycerol binding domain (C1-1), polycomb-like MTF2 factor 2 domain (Mtf2_C), a transactivation domain of FOXO protein family (FOXO-TAD), a homeobox KN domain (Homeobox_KN), a BED zinc finger domain (ZF-BED), a zinc finger of C3HC4-type RING domain (ZF-C3HC4_4), a RAD51 interacting motif domain (RAD51_interact), a p55-binding region of a methyl-CpG-binding domain protein MBD (MBDa), a Notch domain, a Raf-like Ras-binding domain (RBD), a Spin/Ssty family domain (Spin-Ssty), a PHD finger domain (PHD_3), a Low-density lipoprotein receptor domain class A (Ldl_recept_a), a CS domain, a DM DNA-binding domain, and a QLQ domain.

In some embodiments, the effector domain is a protein domain comprising a YAF2_RYBP domain or homeodomain or any combination thereof. In certain embodiments, the homeodomain of the YAF2_RYBP domain is a PRD domain, an NKL domain, a HOXL domain, or a LIM domain. In particular embodiments, the YAF2_RYBP domain may comprise a 32 amino acid Yaf2/RYBP C-terminal binding motif domain (32 aa RYBP).

In some embodiments, the effector domain comprises a protein domain selected from a group consisting of SUMO3 domain, Chromo domain from M phase phosphoprotein 8 (MPP8), chromoshadow domain from Chromobox 1 (CBX1), and SAM_1/SPM domain from Scm Polycomb Group Protein Homolog 1 (SCMH1).

In some embodiments, the effector domain comprises an HNF3 C-terminal domain (HNF_C). The HNF_C domain may be from FOXA1 or FOXA2. In certain embodiments, the HNF_C domain comprises an EH1 (engrailed homology 1) motif.

In some embodiments, the effector domain may comprise an interferon regulatory factor 2-binding protein zinc finger domain (IRF-2BP1_2), a Cyt-b5 domain from DNA repair factor HERC2 E3 ligase, a variant SH3 domain (SH3_9) from Bridging Integrator 1 (BIN1), an HMG-box domain from transcription factor TOX or ZF-C3HC4_2 RING finger domain from the polycomb component PCGF2, a Chromodomain-helicase-DNA binding protein 3 (CHD3) domain, or a ZNF783 domain.

### IV. EPIGENETIC EDITORS

Provided herein are epigenetic editors (i.e., epigenetic editing systems) that direct epigenetic modification(s) to a target sequence in a gene of interest, e.g., using one or more DNA-binding domains as described herein and one or more effector domains (e.g., epigenetic repressor domains) as described herein, in any combination. The DNA-binding domain (in concert with a guide polynucleotide such as one described herein, where the DNA-binding domain is a polynucleotide guided DNA-binding domain) directs the effector domain to epigenetically modify the target sequence, resulting in gene repression or silencing that may be durable and inheritable across cell generations. In some aspects, the epigenetic editors described herein can repress or silence genes reversibly or irreversibly in cells.

In particular embodiments, an epigenetic editor described herein comprises one or more fusion proteins, each comprising (1) DNA-binding domain(s) and (2) effector domain(s). The effector domains may be on one or more fusion proteins comprised by the epigenetic editor. For example, a single fusion protein may comprise all of the effector domains with a DNA-binding domain. Alternatively, the effector domains or subsets thereof may be on separate fusion proteins, each with a DNA-binding domain (which may be the same or different). A fusion protein described herein may further comprise one or more linkers (e.g., peptide linkers), detectable tags, nuclear localization signals (NLSs), or any combination thereof. As used herein, a “fusion protein” refers to a chimeric protein in which two or more coding sequences (e.g., for DNA-binding domain(s) and/or effector domain(s)) are covalently or non-covalently joined, directly or indirectly.

In some embodiments, an epigenetic editor described herein comprises 2, 3, 4, 5, 6, 7, 8, 9, 10, or more effector (e.g., repression/repressor) domains, which may be identical or different. In certain embodiments, two or more of said effector domains function synergistically. Combinations of effector domains may comprise DNA methylation domains, histone deacetylation domains, histone methylation domains, and/or scaffold domains that recruit any of the above. For example, an epigenetic editor described herein may comprise one or more transcriptional repressor domains (e.g., a KRAB domain such as KOX1, ZIM3, ZFP28, or ZN627 KRAB) in combination with one or more DNA methylation domains (e.g., a DNMT domain) and/or recruiter domain (e.g., a DNMT3L domain). Such an epigenetic editor may comprise, for instance, a KRAB domain, a DNMT3A domain, and a DNMT3L domain. In some embodiments, the epigenetic editor further comprises an additional effector domain (e.g., a KAP1, MECP2, HP1b, CBX8, CDYL2, TOX, TOX3, TOX4, EED, RBBP4, RCOR1, or SCML2 domain). In some embodiments, the additional effector domain is a CDYL2, TOX, TOX3, TOX4, or HP1a domain. For example, an epigenetic editor described herein may comprise a CDYL2 and/or a TOX domain in combination with a KRAB domain (e.g., a KOX1 KRAB domain).

### A. Linkers

A fusion protein as described herein may comprise one or more linkers that connect components of the epigenetic editor. A linker may be a peptide or non-peptide linker. In some embodiments, one or more linkers utilized in an epigenetic editor provided herein is a peptide linker, i.e., a linker comprising a peptide moiety. A peptide linker can be any length applicable to the epigenetic editor fusion proteins described herein. In some embodiments, the linker can comprise a peptide between 1 and 200 (e.g., between 1 and 80) amino acids. In some embodiments, the linker comprises from 1 to 5, 1 to 10, 1 to 20, 1 to 30, 1 to 40, 1 to 50, 1 to 60, 1 to 80, 1 to 100, 1 to 150, 1 to 200, 5 to 10, 5 to 20, 5 to 30, 5 to 40, 5 to 60, 5 to 80, 5 to 100, 5 to 150, 5 to 200, 10 to 20, 10 to 30, 10 to 40, 10 to 50, 10 to 60, 10 to 80, 10 to 100, 10 to 150, 10 to 200, 20 to 30, 20 to 40, 20 to 50, 20 to 60, 20 to 80, 20 to 100, 20 to 150, 20 to 200, 30 to 40, 30 to 50, 30 to 60, 30 to 80, 30 to 100, 30 to 150, 30 to 200, 40 to 50, 40 to 60, 40 to 80, 40 to 100, 40 to 150, 40 to 200, 50 to 60 50 to 80, 50 to 100, 50 to 150, 50 to 200, 60 to 80, 60 to 100, 60 to 150, 60 to 200, 80 to 100, 80 to 150, 80 to 200, 100 to 150, 100 to 200, or 150 to 200 amino acids in length. Longer or shorter linkers are also contemplated. In some embodiments, the peptide linker is 3, 4, 5, 6, 7, 8, 9, 10, 11, 12, 13, 14, 15, 16, 17, 18, 19, 20, 25, 30, 25, 40, 45, 50, 55, 60, 65, 70, 75, 80, 85, 90, 95, or 100 amino acids in length. For example, the peptide linker may be 4, 5, 16, 20, 24, 27, 32, 40, 64, 92, or 104 amino acids in length. The peptide linker may be a flexible or rigid linker. In particular embodiments, the peptide linker comprises the amino acid sequence of any one of SEQ ID NOs: 631-637 and 664-666 or a sequence at least 75%, 80%, 85%, 90%, 91%, 92%, 93%, 94%, 95%, 96%, 97%, 98%, or 99% identical thereto.

In certain embodiments, the peptide linker is an XTEN linker. Such a linker may comprise part of the XTEN sequence (Schellenberger et al., Nat Biotechnol (2009) 27 (1): 1186-90), an unstructured hydrophilic polypeptide consisting only of residues G, S, P, T, E, and A. The term “XTEN” as used herein refers to a recombinant peptide or polypeptide lacking hydrophobic amino acid residues. XTEN linkers typically are unstructured and comprise a limited set of natural amino acids. Fusion of XTEN to proteins alters its hydrodynamic properties and reduces the rate of clearance and degradation of the fusion protein. These XTEN fusion proteins are produced using recombinant technology, without the need for chemical modifications, and degraded by natural pathways. The XTEN linker may be, for example, 5, 10, 16, 20, 26, or 80 amino acids in length. In some embodiments, the XTEN linker is 16 amino acids in length. In some embodiments, the XTEN linker is 80 amino acids in length. In certain embodiments, the XTEN linker may be XTEN10, XTEN16, XTEN20, or XTEN80. In certain embodiments, the XTEN linker may comprise the amino acid sequence of any one of SEQ ID NOs: 638-643 or a sequence at least 75%, 80%, 85%, 90%, 91%, 92%, 93%, 94%, 95%, 96%, 97%, 98%, or 99% identical thereto. In particular embodiments, the XTEN linker comprises the amino acid sequence of SEQ ID NO: 638. In particular embodiments, the XTEN linker comprises the amino acid sequence of SEQ ID NO: 643.

In some embodiments, one or more linkers utilized in an epigenetic editor provided herein is a non-peptide linker. For example, the linker may be a carbon bond, a disulfide bond, or carbon-heteroatom bond. In certain embodiments, the linker is a carbon-nitrogen bond of an amide linkage. In certain embodiments, the linker is a cyclic or acyclic, substituted or unsubstituted, or branched or unbranched aliphatic or heteroaliphatic linker. In some embodiments, one or more linkers utilized in an epigenetic editor provided herein is polymeric (e.g., polyethylene, polyethylene glycol, polyamide, polyester, etc.). The linker may comprise, for example, a monomer, dimer, or polymer of aminoalkanoic acid; an aminoalkanoic acid (e.g., glycine, ethanoic acid, alanine, beta-alanine, 3-aminopropanoic acid, 4-aminobutanoic acid, 5-pentanoic acid, etc.); a monomer, dimer, or polymer of aminohexanoic acid (Ahx); or a polyethylene glycol moiety (PEG); or an aryl or heteroaryl moiety. In certain embodiments, the linker may be based on a carbocyclic moiety (e.g., cyclopentane or cyclohexane) or a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, alkyl halides, aryl halides, acyl halides, and isothiocyanates.

Various linker lengths and flexibilities can be employed between any two components of an epigenetic editor (e.g., between an effector domain (e.g., a repressor domain) and a DNA-binding domain (e.g., a Cas9 domain), between a first effector domain and a second effector domain, etc.). The linkers may range from very flexible linkers, such as glycine/serine-rich linkers, to more rigid linkers, in order to achieve the optimal length for effector domain activity for the specific application. In some embodiments, the more flexible linkers are glycine/serine-rich linkers (GS-rich linkers), where more than 45% (e.g., more than 48, 50, 55, 60, 70, 80, or 90%) of the residues are glycine or serine residues. Non-limiting examples of the GS-rich linkers are (GGGGS)n (SEQ ID NO: 664), (G)n (SEQ ID NO: 2078), and W linker (SEQ ID NO: 637). In some embodiments, the more rigid linkers are in the form of the form (EAAAK)n (SEQ ID NO: 665), (SGGS)n (SEQ ID NO: 666), and (XP)n (SEQ ID NO: 2079). In the aforementioned formulae of flexible and rigid linkers, n may be any integer between 1 and 30. In some embodiments, n is 1, 2, 3, 4, 5, 6, 7, 8, 9, 10, 11, 12, 13, 14, or 15. In some embodiments, the linker comprises a (GGS)n (SEQ ID NO: 2080) motif, wherein n is 1, 3, or 7. In some embodiments, the linker comprises a (GGGGS)n motif, wherein n is 4 (SEQ ID NO: 636).

In some embodiments, a linker in an epigenetic editor described herein comprises a nuclear localization signal, for example, with the amino acid sequence of any one of SEQ ID NOs: 644-649. In some embodiments, a linker in an epigenetic editor described herein comprises an expression tag, e.g., a detectable tag such as a green fluorescent protein.

### B. Nuclear Localization Signals

A fusion protein described herein may comprise one or more nuclear localization signals, and in certain embodiments, may comprise two or more nuclear localization signals. For example, the fusion protein may comprise 1, 2, 3, 4, 5, 6, 7, 8, 9, 10, or more nuclear localization signals. As used herein, a “nuclear localization signal” (NLS) is an amino acid sequence that directs proteins to the nucleus. In certain embodiments, the NLS may be an SV40 NLS (e.g., with the amino acid sequence of SEQ ID NO: 644). The fusion protein may comprise an NLS at its N-terminus, C-terminus, or both, and/or an NLS may be embedded in the middle of the fusion protein (e.g., at the N- or C-terminus of a DNA-binding domain or an effector domain).

In some embodiments, the fusion protein may comprise two NLSs. The fusion protein may comprise two NLSs at its N-terminus or C-terminus. The fusion protein may comprise one NLS located at its N-terminus and one NLS embedded in the middle of the fusion protein, or one NLS located at its C-terminus and one NLS embedded in the middle of the fusion protein. The fusion protein may comprise two NLSs embedded in the middle of the fusion protein.

In some embodiments, the fusion protein may comprise four NLSs. The fusion protein may comprise at least two (e.g., two, three, or four) NLSs at its N-terminus or C-terminus. The fusion protein may comprise at least one (e.g., one, two, three, or four) NLSs embedded in the middle of the fusion protein. In particular embodiments, the fusion protein may comprise two NLSs at its N-terminus and two NLSs at its C-terminus.

An NLS described herein may be an endogenous NLS sequence. In certain embodiments, an NLS described herein comprises the amino acid sequence of any one of SEQ ID NOs: 644-649, or a sequence at least 75%, 80%, 85%, 90%, 91%, 92%, 93%, 94%, 95%, 96%, 97%, 98%, or 99% identical to the selected sequence. In particular embodiments, the NLS comprises the amino acid sequence of SEQ ID NO: 644. Additional NLSs are known in the art.

In some embodiments, an epigenetic editor comprising a fusion protein that comprises at least one NLS at the N-terminus and at least one NLS at the C-terminus may increase the efficiency of the epigenetic editor by at least 5%, at least 10%, at least 15%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1,000%, at least 5,000%, at least 10,000%, at least 50,000%, at least 100,000%, or more as compared to an epigenetic editor with a corresponding fusion protein that does not have at least one NLS at the N-terminus and at least one NLS at the C-terminus.

In some embodiments, an epigenetic editor comprising a fusion protein that comprises two NLSs at the N-terminus and two NLSs at the C-terminus may increase the efficiency of the epigenetic editor by at least 5%, at least 10%, at least 15%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1,000%, at least 5,000%, at least 10,000%, at least 50,000%, at least 100,000%, or more as compared to an epigenetic editor with a corresponding fusion protein that does not have two NLSs at the N-terminus and two NLSs at the C-terminus.

### C. Tags

Epigenetic editors provided herein may comprise one or more additional sequences (“tags”) for tracking, detection, and localization of the editors. In some embodiments, the epigenetic editor comprises 1, 2, 3, 4, 5, 6, 7, 8, 9, 10 or more detectable tags. Each of the detectable tags may be the same or different.

For example, an epigenetic editor fusion protein may comprise cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, poly-histidine tags (also referred to as histidine tags or His-tags), maltose binding protein (MBP)-tags, nus-tags, glutathione-S-transferase (GST)-tags, green fluorescent protein (GFP)-tags, thioredoxin-tags, S-tags, Softags (e.g., Softag 1 or Softag 3), strep-tags, biotin ligase tags, FlAsH tags, V5 tags, and SBP-tags. Additional suitable sequences will be apparent to those of skill in the art.

### D. Fusion Protein Configurations

A fusion protein of an epigenetic editor described herein may have its components structured in different configurations. For example, the DNA-binding domain may be at the C-terminus, the N-terminus, or in between two or more epigenetic effector domains or additional domains. In some embodiments, the DNA-binding domain is at the C-terminus of the epigenetic editor. In some embodiments, the DNA-binding domain is at the N-terminus of the epigenetic editor. In some embodiments, the DNA-binding domain is linked to one or more nuclear localization signals. In some embodiments, the DNA-binding domain is flanked by an epigenetic effector domain and/or an additional domain on both sides. In some embodiments, where “DBD” indicates DNA-binding domain and “ED” indicates effector domain, the epigenetic editor comprises the configuration of:
N′]-[ED1]-[DBD]-[ED2]-[C′
N′]-[ED1]-[DBD]-[ED2]-[ED3]-[C′
N′]-[ED1]-[ED2]-[DBD]-[ED3]-[C′
or
N′]-[ED1]-[ED2]-DBD]-[ED3]-[ED4]-[C′.

In some embodiments, an epigenetic editor comprises a DNA-binding domain (DBD), a DNA methyltransferase (DNMT) domain, and a transcriptional repressor (“repressor”) domain that represses or silences expression of a target gene. The DBD, DNMT, and transcriptional repressor domains may be any as described herein, in any combination. The DBD, DNMT domain, and repressor domain may be in any configuration, e.g., with any of said domains at the N-terminus, at the C-terminus, or in the middle of the fusion protein. In some embodiments, the epigenetic editor comprises a fusion protein with the configuration of:
N′]-[DNMT domain]-[DBD]-[repressor domain]-[C′
N′]-[repressor domain]-[DBD]-[DNMT domain]-[C′
N′]-[DNMT domain]-[repressor domain]-[DBD]-[C′
or
N′]-[repressor domain]-[DNMT domain]-[DBD]-[C′.

In some embodiments, a connecting structure “]-[” in any one of the epigenetic editor structures is a linker, e.g., a peptide linker; a detectable tag; a peptide bond; a nuclear localization signal; and/or a promoter or regulatory sequence. In an epigenetic editor structure, the multiple connecting structures “]-[” may be the same or may each be a different linker, tag, NLS, or peptide bond. In some embodiments, the DNMT domain may comprise any one of the domains in Table 7, or any combinations or homologs thereof. In particular embodiments, the DNMT domain comprises DNMT3A or a truncated version thereof, DNMT3L or a truncated version thereof, or both. In particular embodiments, the DBD is a catalytically inactive polynucleotide guided DNA-binding domain (e.g., a dCas9) or a ZFP domain. In certain embodiments, the repressor domain comprises any one of the domains shown in Table 5 or 6, or any combinations or homologs thereof. For example, the repressor domain may be a KRAB domain. In certain embodiments, the repressor domain is a ZFP28, ZN627, KAP1, MeCP2, HP1b, CBX8, CDYL2, TOX, Tox3, Tox4, EED, RBBP4, RCOR1, or SCML2 domain, or a fusion of two of said domains (e.g., a fusion of the N- and C-terminal regions of ZIM3 and KOX1 KRAB). In particular embodiments, the repressor domain is a KRAB domain from ZFP28, ZN627, ZIM3, or KOX1.

In some embodiments, the epigenetic editor comprises a configuration selected from
N′]-[DNMT3A-DNMT3L]-[DBD]-[repressor]-[C′
N′]-[repressor]-[DBD]-[DNMT3A-DNMT3L]-[C′
N′]-[repressor]-[DBD]-[DNMT3A]-[C′
N]-[DNMT3A]-[DBD]-[repressor]-[C′
N′]-[repressor]-[DBD]-[DNMT3A]-[DNMT3L]-[C′
N′]-[DNMT3A]-[DNMT3L]-[DBD]-[repressor]-[C′
N′]-[DNMT3A]-[DBD]-[C′
N′]-[DBD]-[DNMT3A]-[C′
N′]-[DNMT3L]-[DBD]-[C′
N′]-[DBD]-[DNMT3L]-[C′
   wherein [DNMT3A-DNMT3L] indicates that the DNMT3A and DNMT3L domains are directly fused via a peptide bond, and wherein the connecting structure ]-[ is any one of the linkers as described herein, a detectable tag, an affinity domain, a peptide bond, a nuclear localization signal, a promoter, and/or a regulatory sequence. The DBD, repressor, DNMT3A, and DNMT3L domains may be any as described herein, in any combination. For example, the DNMT3A and DNMT3L domains may be selected from those in Table 7. In particular embodiments, the DBD is a CRISPR-associated protein domain (e.g., dCas9) or a ZFP domain; the repressor domain is a KRAB domain derived from KOX1, ZIM3, ZFP28, or ZN627; the DNMT3A domain is a human DNMT3A domain; and the DNMT3L domain is a human or mouse DNMT3L domain; any combination of these components is also contemplated by the present disclosure.

In some embodiments, the epigenetic editor comprises a configuration selected from
N′]-[DNMT3A]-[DBD]-[SETDB1]-[C′
N′]-[DNMT3A]-[DNMT3L]-[DBD]-[SETDB1]-[C′
N′]-[DNMT3A-DNMT3L]-[DBD]-[SETDB1]-[C′
N′]-[SETDB1]-[DBD]-[DNMT3A]-[DNMT3L]-[C′
N′]-[SETDB1]-[DBD]-[DNMT3A]-[C′
   wherein [DNMT3A-DNMT3L] indicates that the DNMT3A and DNMT3L domains are directly fused via a peptide bond, and wherein the connecting structure ]-[ is any one of the linkers as described herein, a detectable tag, an affinity domain, a peptide bond, a nuclear localization signal, a promoter, and/or a regulatory sequence. The DBD, SETDB1, DNMT3A, and DNMT3L domains may be any as described herein, in any combination. In particular embodiments, the DBD is a CRISPR-associated protein domain (e.g., dCas9) or a ZFP domain; the SETDB 1 domain is derived from human SETDB1, ZIM3, ZFP28, or ZN627; the DNMT3A domain is a human DNMT3A domain; and the DNMT3L domain is a human or mouse DNMT3L domain; any combination of these components is also contemplated by the present disclosure.

Particular constructs contemplated herein include:
DNMT3A-DNMT3L-XTEN80-NLS-dCas9-NLS-XTEN16-KOX1 KRAB (Configuration 1),
DNMT3A-DNMT3L-XTEN80-NLS-ZFP domain-NLS-XTEN16-KOX1 KRAB (Configuration 2),
NLS-DNMT3A-DNMT3L-XTEN80-dCas9-XTEN16-KOX1 KRAB-NLS (Configuration 3),
NLS-DNMT3A-DNMT3L-XTEN80-ZFP domain-XTEN16-KOX1 KRAB-NLS (Configuration 4),
NLS-NLS-DNMT3A-DNMT3L-XTEN80-dCas9-XTEN16-KOX1 KRAB-NLS-NLS (Configuration 5), and
NLS-NLS-DNMT3A-DNMT3L-XTEN80-ZFP domain-XTEN16-KOX1 KRAB-NLS-NLS (Configuration 6).

The DNMT3L and DNMT3A may be derived from human parental proteins, mouse parental proteins, or any combination thereof. In certain embodiments, the DNMT3L and DNMT3A are derived from mouse and human parental proteins, respectively (mDNMT3L and hDNMT3A). In certain embodiments, the DNMT3L and DNMT3A are both derived from human parental proteins (hDNMT3L and hDNMT3A). In some embodiments, the dCas9 is dSpCas9. In some embodiments, the KOX1 is human KOX1. Also contemplated is any of Configurations 1-6 wherein the KOX1 KRAB domain is replaced by a ZFP28, ZN627, or ZIM3 KRAB domain. In some embodiments, the ZFP28, ZN627, and ZIM3 are human ZFP28, ZN627, and ZIM3, respectively. In particular embodiments, the fusion construct may have the configuration:
NLS-NLS-hDNMT3A-hDNMT3L-XTEN80-dCas9-XTEN16-KOX1 KRAB-NLS-NLS (Configuration 7),
NLS-NLS-DNMT3A-DNMT3L-XTEN80-ZFP domain-XTEN16-KOX1 KRAB-NLS-NLS (Configuration 8),
NLS-NLS-hDNMT3A-hDNMT3L-XTEN80-dCas9-XTEN16-ZFP28 KRAB-NLS-NLS (Configuration 9),
NLS-NLS-DNMT3A-DNMT3L-XTEN80-ZFP domain-XTEN16-ZFP28 KRAB-NLS-NLS (Configuration 10),
NLS-NLS-hDNMT3A-hDNMT3L-XTEN80-dCas9-XTEN16-ZN627 KRAB-NLS-NLS (Configuration 11),
NLS-NLS-DNMT3A-DNMT3L-XTEN80-ZFP domain-XTEN16-ZN627 KRAB-NLS-NLS (Configuration 12),
NLS-NLS-hDNMT3A-hDNMT3L-XTEN80-dCas9-XTEN16-ZIM3 KRAB-NLS-NLS (Configuration 13), or
NLS-NLS-DNMT3A-DNMT3L-XTEN80-ZFP domain-XTEN16-ZIM3 KRAB-NLS-NLS (Configuration 14).

In particular embodiments, a fusion construct described herein may have Configuration 1 and comprise SEQ ID NO: 658, or a sequence at least 75%, 80%, 85%, 90%, 91%, 92%, 93%, 94%, 95%, 96%, 97%, 98%, or 99% identical thereto. In SEQ ID NO: 658 below, the XTEN linkers are underlined, the W linker is bolded, underlined, and italicized, the NLS sequences are bolded, the DNMT3A sequence is italicized, the DNMT3L sequence is underlined and italicized, the dCas9 domain is bolded and italicized, and the KOX1 KRAB domain is underlined and bolded:

(SEQ ID NO: 658)
MNHDQEFDPPKVYPPVPAEKRKPIRVLSLFDGIATGLLVLKDLGIQVDRYIASEVCE
DSITVGMVRHQGKIMYVGDVRSVTQKHIQEWGPFDLVIGGSPCNDLSIVNPARKGLY
EGTGRLFFEFYRLLHDARPKEGDDRPFFWLFENVVAMGVSDKRDISRFLESNPVMID
AKEVSAAHRARYFWGNLPGMNRPLASTVNDKLELQECLEHGRIAKFSKVRTITTRSN
SIKQGKDQHFPVFMNEKEDILWCTEMERVFGFPVHYTDVSNMSRLARQRLLGRSWSV
PVIRHLFAPLKEYFACVSSGNSNANSRGPSFSSGLVPLSLRGSHMGPMEIYKTVSAW
KRQPVRVLSLFRNIDKVLKSLGFLESGSGSGGGTLKYVEDVTNVVRRDVEKWGPFDL
VYGSTQPLGSSCDRCPGWYMFQFHRILQYALPRQESQRPFFWIFMDNLLLTEDDQET
TTRFLQTEAVTLQDVRGRDYQNAMRVWSNIPGLKSKHAPLTPKEEEYLQAQVRSRSK
LDAPKVDLLVKNCLLPLREYFKYFSQNSLPLGGPSSGAPPPSGGSPAGSPTSTEEGT
SESATPESGPGTSTEPSEGSAPGSPAGSPTSTEEGTSTEPSEGSAPGTSTEPSEPKK
KRKVYMDKKYSIGLAIGTNSVGWAVITDEYKVPSKKFKVLGNTDRHSIKKNLIGALL
FDSGETAEATRLKRTARRRYTRRKNRICYLQEIFSNEMAKVDDSFFHRLEESELVEE
DKKHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRRLE
NLIAQLPGEKKNGLFGNLIALSLGLTPNFKSNEDLAEDAKLQLSKDTYDDDLDNLLA
QIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLN
REDLLRKQRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKNLPNEKVL
PKHSLLYEYFTVYNELTKVKYVTEGMRKPAFLSGEQKKAIVDLLFKTNRKVTVKQLK
EDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTI
LDFLKSDGFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEEGIKELGS
QILKEHPVENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVS
DFRKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDKGR
DFATVRKVLSMPQVNIVKKTEVQTGGFSKESILPKRNSDKLIARKKDWDPKKYGGFD
SPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSP
EDNEQKQLFVEQHKHYLDEIIEQISEFSKRVILADANLDKVLSAYNKHRDKPIREQA
ENIIHLFTLTNLGAPAAFKYFDTTIDRKRYTSTKEVLDATLIHQSITGLYETRIDLS
QLGGDPKKKRKVSGSETPGTSESATPESTGRTLVTFKDVFVDETREEWKLLDTAQQI
VYRNVMLENYKNLVSLGYQLTKPDVILRLEKGEEP

In particular embodiments, a fusion construct described herein may comprise the sequence provided below (SEQ ID NO: 1495), or a sequence at least 75%, 80%, 85%, 90%, 91%, 92%, 93%, 94%, 95%, 96%, 97%, 98%, or 99% identical thereto. In SEQ ID NO: 1495 below, the XTEN linkers are underlined, the W linker is bolded, underlined, and italicized, the NLS sequences are bolded, the DNMT3A sequence is italicized, the DNMT3L sequence is underlined and italicized, the dCas9 domain is bolded and italicized, and the KOX1 KRAB domain is underlined and bolded:

(SEQ ID NO: 1495)
MNHDQEFDPPKVYPPVPAEKRKPIRVLSLFDGIATGLLVLKDLGIQVDRYIASEVCE
DSITVGMVRHQGKIMYVGDVRSVTQKHIQEWGPFDLVIGGSPCNDLSIVNPARKGLY
EGTGRLFFEFYRLLHDARPKEGDDRPFFWLFENVVAMGVSDKRDISRFLESNPVMID
AKEVSAAHRARYFWGNLPGMNRPLASTVNDKLELQECLEHGRIAKFSKVRTITTRSN
SIKQGKDQHFPVFMNEKEDILWCTEMERVFGFPVHYTDVSNMSRLARQRLLGRSWSV
PVIRHLFAPLKEYFACVSSGNSNANSRGPSFSSGLVPLSLRGSHMNPLEMFETVPVW
RRQPVRVLSLFEDIKKELTSLGFLESGSDPGQLKHVVDVTDTVRKDVEEWGPFDLVY
GATPPLGHTCDRPPSWYLFQFHRLLQYARPKPGSPRPFFWMFVDNLVLNKEDLDVAS
RFLEMEPVTIPDVHGGSLQNAVRVWSNIPAIRSRHWALVSEEELSLLAQNKQSSKLA
AKWPTKLVKNCFLPLREYFKYFSTELTSSLGGPSSGAPPPSGGSPAGSPTSTEEGTS
ESATPESGPGTSTEPSEGSAPGSPAGSPTSTEEGTSTEPSEGSAPGTSTEPSEPKKK
RKVYMDKKYSIGLAIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRGH
FLIEGDLNPDNSDVDKLFIQLVQTYNQLFEENPINASGVDAKAILSARLSKSRRLEN
LIAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLNR
EDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTL
TLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGI
LQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLTKAERGGL
SELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFDS
PTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPE
DNEQKQLFVEQHKHYLDEIIEQISEFSKRVILADANLDKVLSAYNKHRDKPIREQAE
NIIHLFTLTNLGAPAAFKYFDTTIDRKRYTSTKEVLDATLIHQSITGLYETRIDLSQ
LGGDPKKKRKVSGSETPGTSESATPESTGRTLVTFKDVFVDFTREEWKLLDTAQQIV
YRNVMLENYKNLVSLGYQLTKPDVILRLEKGEEP

In particular embodiments, a fusion construct described herein may have Configuration 2 and comprise SEQ ID NO: 659, or a sequence at least 75%, 80%, 85%, 90%, 91%, 92%, 93%, 94%, 95%, 96%, 97%, 98%, or 99% identical thereto. In SEQ ID NO: 659 below, the XTEN linkers are underlined, the W linker is bolded, underlined, and italicized, the NLS sequences are bolded and underlined, the DNMT3A sequence is italicized, the DNMT3L sequence is underlined and italicized, the ZFP domain is bolded, and the KOX1 KRAB domain is underlined and bolded. Variable amino acids represented by Xs are the amino acids of the DNA-recognition helix of the zinc finger and XX in italics may be either TR, LR or LK.

(SEQ ID NO: 659)
MNHDQEFDPPKVYPPVPAEKRKPIRVLSLFDGIATGLLVLKDLGIQVDRYIASEVCE
DSITVGMVRHQGKIMYVGDVRSVTQKHIQEWGPFDLVIGGSPCNDLSIVNPARKGLY
EGTGRLFFEFYRLLHDARPKEGDDRPFFWLFENVVAMGVSDKRDISRFLESNPVMID
AKEVSAAHRARYFWGNLPGMNRPLASTVNDKLELQECLEHGRIAKFSKVRTITTRSN
SIKQGKDQHFPVFMNEKEDILWCTEMERVFGFPVHYTDVSNMSRLARQRLLGRSWSV
PVIRHLFAPLKEYFACVSSGNSNANSRGPSFSSGLVPLSLRGSHMGPMEIYKTVSAW
KRQPVRVLSLFRNIDKVLKSLGFLESGSGSGGGTLKYVEDVTNVVRRDVEKWGPFDL
VYGSTQPLGSSCDRCPGWYMFQFHRILQYALPRQESQRPFFWIFMDNLLLTEDDQET
TTRFLQTEAVTLQDVRGRDYQNAMRVWSNIPGLKSKHAPLTPKEEEYLQAQVRSRSK
LDAPKVDLLVKNCLLPLREYFKYFSQNSLPLGGPSSGAPPPSGGSPAGSPTSTEEGT
SESATPESGPGTSTEPSEGSAPGSPAGSPTSTEEGTSTEPSEGSAPGTSTEPSEPKK
KRKVYSRPGERPFQCRICMRNFSXXXXXXXHXXTHTGEKPFQCRICMRNFSXXXXXX
XHXXTH[linker]PFQCRICMRNFSXXXXXXXXXXTHTGEKPFQCRICMRNFSXXX
XXXXHXXTH[linker]PFQCRICMRNFSXXXXXXXXXXTHTGEKPFQCRICMRNFS
XXXXXXXXXXTHLRGSPKKKRKVSGSETPGTSESATPESTGRTLVTFKDVFVDFTRE
EWKLLDTAQQIVYRNVMLENYKNLVSLGYQLTKPDVILRLEKGEEP

In certain embodiments, the six “XXXXXXX” regions in SEQ ID NO: 659 comprise amino acid sequences that form a zinc finger. In the sequence above, [linker] represents a linker sequence. In some embodiments, one or both linker sequences may be TGSQKP (SEQ ID NO: 651). In some embodiments, one or both linker sequences may be TGGGGSQKP (SEQ ID NO: 652). In some embodiments, one linker sequence may have the amino acid sequence of SEQ ID NO: 651 and the other linker sequence may have the amino acid sequence of SEQ ID NO: 652.

In particular embodiments, a fusion construct described herein may comprise the sequence provided below (SEQ ID NO: 1496), or a sequence at least 75%, 80%, 85%, 90%, 91%, 92%, 93%, 94%, 95%, 96%, 97%, 98%, or 99% identical thereto. In SEQ ID NO: 1496 below, the XTEN linkers are underlined, the W linker is bolded, underlined, and italicized, the NLS sequences are bolded and underlined, the DNMT3A sequence is italicized, the DNMT3L sequence is underlined and italicized, the ZFP domain is bolded, and the KOX1 KRAB domain is underlined and bolded. Variable amino acids represented by Xs are the amino acids of the DNA-recognition helix of the zinc finger and XX in italics may be either TR, LR or LK.

(SEQ ID NO: 1496)
MNHDQEFDPPKVYPPVPAEKRKPIRVLSLFDGIATGLLVLKDLGIQVDRYIASEVCE
DSITVGMVRHQGKIMYVGDVRSVTQKHIQEWGPFDLVIGGSPCNDLSIVNPARKGLY
EGTGRLFFEFYRLLHDARPKEGDDRPFFWLFENVVAMGVSDKRDISRFLESNPVMID
AKEVSAAHRARYFWGNLPGMNRPLASTVNDKLELQECLEHGRIAKFSKVRTITTRSN
SIKQGKDQHFPVFMNEKEDILWCTEMERVFGFPVHYTDVSNMSRLARQRLLGRSWSV
PVIRHLFAPLKEYFACVSSGNSNANSRGPSFSSGLVPLSLRGSHMNPLEMFETVPVW
RRQPVRVLSLFEDIKKELTSLGFLESGSDPGQLKHVVDVTDTVRKDVEEWGPFDLVY
GATPPLGHTCDRPPSWYLFQFHRLLQYARPKPGSPRPFFWMFVDNLVLNKEDLDVAS
RFLEMEPVTIPDVHGGSLQNAVRVWSNIPAIRSRHWALVSEEELSLLAONKQSSKLA
AKWPTKLVKNCFLPLREYFKYFSTELTSSLGGPSSGAPPPSGGSPAGSPTSTEEGTS
ESATPESGPGTSTEPSEGSAPGSPAGSPTSTEEGTSTEPSEGSAPGTSTEPSEPKKK
RKVYSRPGERPFQCRICMRNFSXXXXXXXHXXTHTGEKPFQCRICMRNFSXXXXXXX
HXXTH[linker]PFQCRICMRNFSXXXXXXXXXXTHTGEKPFQCRICMRNFSXXXX
XXXHXXTH[linker]PFQCRICMRNFSXXXXXXXHXXTHTGEKPFQCRICMRNFSX
XXXXXXXXXTHLRGSPKKKRKVSGSETPGTSESATPESTGRTLVTFKDVFVDFTREE
WKLLDTAQQIVYRNVMLENYKNLVSLGYOLTKPDVILRLEKGEEP

In certain embodiments, the six “XXXXXXX” regions in SEQ ID NO: 1496 comprise, in order, the F1-F6 amino acid sequences shown in Tables 1-3 for any one of ZF001-ZF048. [linker] represents a linker sequence. In some embodiments, one or both linker sequences may be TGSQKP (SEQ ID NO: 651). In some embodiments, one or both linker sequences may be TGGGGSQKP (SEQ ID NO: 652). In some embodiments, one linker sequence may have the amino acid sequence of SEQ ID NO: 651 and the other linker sequence may have the amino acid sequence of SEQ ID NO: 652.

In some embodiments, the fusion protein may further comprise a Dnmt3A ADD domain, e.g., downstream of the Dnmt3A domain sequence disclosed in SEQ ID Nos 658, 659, 1495, or 1496 disclosed above. In some embodiments, the ADD sequence is situated between the Dnmt3A and the Dnmt3L sequence of the fusion protein. In some embodiments, the ADD sequence is at the C-terminal end of the Dnmt3A domain. In some embodiments, the Dnmt3A sequence and the ADD sequence are separated by a linker, e.g., a linker disclosed herein. In some embodiments, the ADD sequence and the Dnmt3L sequence are separated by a linker, e.g., a linker disclosed herein. In some embodiments, the ADD domain comprises the sequence:

(SEQ ID NO: 1497)
MAAIPALDPEAEPSMDVILVGSSELSSSVSPGTGRDLIAYEVKANQRNIE
DICICCGSLQVHTQHPLFEGGICAPCKDKFLDALFLYDDDGYQSYCSICC
SGETLLICGNPDCTRCYCFECVDSLVGPGTSGKVHAMSNWVCYLCLPSSR
SGLLORRRKWRSQLKAFYDRESENPLEMFETVPVWRRQPVRVLSLFEDIK
KELTSLGFLESGSDPGQLKHVVDVTDTVRKDVEEWGPFDLVYGATPPLGH
TCDRPPSWYLFQFHRLLQYARPKPGSPRPFFWMFVDNLVLNKEDLDVASR
FLEMEPVTIPDVHGGSLQNAVRVWSNIPAIRSRHWALVSEEELSLLAQNK
QSSKLAAKWPTKLVKNCFLPLREYFKYFSTELTSSL.

In particular embodiments, a fusion construct described herein may have Configuration 7 and comprise SEQ ID NO: 660, or a sequence at least 75%, 80%, 85%, 90%, 91%, 92%, 93%, 94%, 95%, 96%, 97%, 98%, or 99% identical thereto.

In particular embodiments, a fusion construct described herein may have Configuration 9 and comprise SEQ ID NO: 661, or a sequence at least 75%, 80%, 85%, 90%, 91%, 92%, 93%, 94%, 95%, 96%, 97%, 98%, or 99% identical thereto.

In particular embodiments, a fusion construct described herein may have Configuration 11 and comprise SEQ ID NO: 662, or a sequence at least 75%, 80%, 85%, 90%, 91%, 92%, 93%, 94%, 95%, 96%, 97%, 98%, or 99% identical thereto.

In particular embodiments, a fusion construct described herein may have Configuration 13 and comprise SEQ ID NO: 663, or a sequence at least 75%, 80%, 85%, 90%, 91%, 92%, 93%, 94%, 95%, 96%, 97%, 98%, or 99% identical thereto.

In some embodiments, a fusion construct described herein (e.g., the fusion construct of any one of Configurations 1-14) is within an expression construct that comprises a WPRE sequence, a polyadenylation site, or both. In certain embodiments, the WPRE sequence is in a 3′ noncoding region. In certain embodiments, the WPRE sequence is upstream from a poly-adenylation site. In particular embodiments, the expression construct comprises the fusion construct (e.g., of any one of Configurations 1-14) and a WPRE sequence in a 3′ noncoding region upstream from a polyadenylation site.

### V. MULTIPLEX EPIGENETIC REGULATION

Multiple fusion proteins may be used to effect activation or repression of a target gene or multiple target genes. For example, an epigenetic editor fusion protein comprising a DNA-binding domain (e.g., a dCas9 domain) and an effector domain may be co-delivered with two or more guide polynucleotides (e.g., gRNAs), each targeting a different target DNA sequence. The target sites for two of the DNA-binding domains may be the same or in the vicinity of each other, or separated by, for example, about 100 base pairs, about 200 base pairs, about 300 base pairs, about 400 base pairs, about 500 base pairs, or about 600 or more base pairs. In addition, when targeting double-strand DNA, such as an endogenous gene locus, the guide polynucleotides may target the same or different strands (one or more to the positive strand and/or one or more to the negative strand).

In some embodiments, multiplex epigenetic regulation comprises the use of one epigenetic editor per target gene. In some embodiments, multiple epigenetic regulation comprises the use of two or more epigenetic editors per target gene. In some embodiments, one epigenetic editor is used to target some target genes and more than one epigenetic editor is used to target one or more other target genes. In some embodiments, a single epigenetic editor fusion protein is used to target all target genes.

In some embodiments, an epigenetic editor is used in combination with epigenetic editor(s) targeting B2M, RFX5, RFXAP, CD3D, CD3G, TRAC, CD247, TRBC, CIITA, PDCDI, TIM-3, TIGIT, LAG3, CTLA4, AAVSI, CCR5, TET2, TGFBR2, A2AR, CISH, PTPN11, PTPN6, PTPA, PTPN2, JUNB, TOX, TOX2, NR4A1, NR4A2, NR4A3, MAP4K1, REL, IRF4, DGKA, PIK3CD, HLA-A, USP16, DCK, FAS, or any combination thereof.

In some embodiments, two epigenetic editors are used in combination. In some embodiments, an epigenetic editor targeting B2M is used in combination with an epigenetic editor targeting CIITA. In some embodiments, an epigenetic editor targeting B2M is used in combination with an epigenetic editor targeting TRAC. In some embodiments, an epigenetic editor targeting B2M is used in combination with an epigenetic editor targeting RFX5. In some embodiments, an epigenetic editor targeting B2M is used in combination with an epigenetic editor targeting RFXAP. In some embodiments, an epigenetic editor targeting B2M is used in combination with an epigenetic editor targeting CD3D. In some embodiments, an epigenetic editor targeting B2M is used in combination with an epigenetic editor targeting CD3G. In some embodiments, an epigenetic editor targeting B2M is used in combination with an epigenetic editor targeting CD3D/G-CRE. In some embodiments, an epigenetic editor targeting B2M is used in combination with an epigenetic editor targeting CD247. In some embodiments, an epigenetic editor targeting CIITA is used in combination with an epigenetic editor targeting TRAC. In some embodiments, an epigenetic editor targeting CIITA is used in combination with an epigenetic editor targeting RFX5. In some embodiments, an epigenetic editor targeting CIITA is used in combination with an epigenetic editor targeting RFXAP. In some embodiments, an epigenetic editor targeting CIITA is used in combination with an epigenetic editor targeting CD3D. In some embodiments, an epigenetic editor targeting CIITA is used in combination with an epigenetic editor targeting CD3G. In some embodiments, an epigenetic editor targeting CIITA is used in combination with an epigenetic editor targeting CD3D/G-CRE. In some embodiments, an epigenetic editor targeting CIITA is used in combination with an epigenetic editor targeting CD247. In some embodiments, an epigenetic editor targeting TRAC is used in combination with an epigenetic editor targeting RFX5. In some embodiments, an epigenetic editor targeting TRAC is used in combination with an epigenetic editor targeting RFXAP. In some embodiments, an epigenetic editor targeting TRAC is used in combination with an epigenetic editor targeting CD3D. In some embodiments, an epigenetic editor targeting TRAC is used in combination with an epigenetic editor targeting CD3G. In some embodiments, an epigenetic editor targeting TRAC is used in combination with an epigenetic editor targeting CD3D/G-CRE. In some embodiments, an epigenetic editor targeting TRAC is used in combination with an epigenetic editor targeting CD247. In some embodiments, an epigenetic editor targeting RFX5 is used in combination with an epigenetic editor targeting CD3D. In some embodiments, an epigenetic editor targeting RFX5 is used in combination with an epigenetic editor targeting RFXAP. In some embodiments, an epigenetic editor targeting RFX5 is used in combination with an epigenetic editor targeting CD3G. In some embodiments, an epigenetic editor targeting RFX5 is used in combination with an epigenetic editor targeting CD3D/G. In some embodiments, an epigenetic editor targeting RFX5 is used in combination with an epigenetic editor targeting CD247. In some embodiments, an epigenetic editor targeting CD3D is used in combination with an epigenetic editor targeting CD247. In some embodiments, an epigenetic editor targeting CD3G is used in combination with an epigenetic editor targeting CD247. In some embodiments, an epigenetic editor targeting CDC3D/G-CRE is used in combination with an epigenetic editor targeting CD247. In some embodiments, an epigenetic editor targeting CD3D/G-CRE is used in combination with an epigenetic editor targeting CD247.

In some embodiments, three epigenetic editors are used in combination. In some embodiments, an epigenetic editor targeting B2M is used in combination with an epigenetic editor targeting CIITA and an epigenetic editor targeting TRAC. In some embodiments, an epigenetic editor targeting B2M is used in combination with an epigenetic editor targeting CIITA and an epigenetic editor targeting RFX5. In some embodiments, an epigenetic editor targeting B2M is used in combination with an epigenetic editor targeting CIITA and an epigenetic editor targeting CD3D. In some embodiments, an epigenetic editor targeting B2M is used in combination with an epigenetic editor targeting CIITA and an epigenetic editor targeting CD3G. In some embodiments, an epigenetic editor targeting B2M is used in combination with an epigenetic editor targeting CIITA and an epigenetic editor targeting CD3D/G-CRE. In some embodiments, an epigenetic editor targeting B2M is used in combination with an epigenetic editor targeting CIITA and an epigenetic editor targeting CD247. In some embodiments, an epigenetic editor targeting B2M is used in combination with an epigenetic editor targeting RFX5 and an epigenetic editor targeting CD3D. In some embodiments, an epigenetic editor targeting B2M is used in combination with an epigenetic editor targeting RFX5 and an epigenetic editor targeting CD3G. In some embodiments, an epigenetic editor targeting B2M is used in combination with an epigenetic editor targeting RFX5 and an epigenetic editor targeting CD3D/G-CRE. In some embodiments, an epigenetic editor targeting B2M is used in combination with an epigenetic editor targeting RFXAP and an epigenetic editor targeting CD3D. In some embodiments, an epigenetic editor targeting B2M is used in combination with an epigenetic editor targeting RFXAP and an epigenetic editor targeting CD3G. In some embodiments, an epigenetic editor targeting B2M is used in combination with an epigenetic editor targeting RFXAP and an epigenetic editor targeting CD3D/G-CRE. In some embodiments, an epigenetic editor targeting B2M is used in combination with an epigenetic editor targeting RFX5 and an epigenetic editor targeting CD247. In some embodiments, an epigenetic editor targeting B2M is used in combination with an epigenetic editor targeting RFX5 and an epigenetic editor targeting TRAC. In some embodiments, an epigenetic editor targeting B2M is used in combination with an epigenetic editor targeting CD3D and an epigenetic editor targeting CD247. In some embodiments, an epigenetic editor targeting B2M is used in combination with an epigenetic editor targeting CD3G and an epigenetic editor targeting CD247. In some embodiments, an epigenetic editor targeting B2M is used in combination with an epigenetic editor targeting CD3D/G-CRE and an epigenetic editor targeting CD247. In some embodiments, an epigenetic editor targeting B2M is used in combination with an epigenetic editor targeting CD3G and an epigenetic editor targeting CD3D. In some embodiments, an epigenetic editor targeting B2M is used in combination with an epigenetic editor targeting CD3D/G-CRE and an epigenetic editor targeting CD3D. In some embodiments, an epigenetic editor targeting B2M is used in combination with an epigenetic editor targeting CD3D/G-CRE and an epigenetic editor targeting CD3G. In some embodiments, an epigenetic editor targeting CIITA is used in combination with an epigenetic editor targeting TRAC and an epigenetic editor targeting RFX5. In some embodiments, an epigenetic editor targeting CIITA is used in combination with an epigenetic editor targeting TRAC and an epigenetic editor targeting CD3D. In some embodiments, an epigenetic editor targeting CIITA is used in combination with an epigenetic editor targeting TRAC and an epigenetic editor targeting CD3G. In some embodiments, an epigenetic editor targeting CIITA is used in combination with an epigenetic editor targeting TRAC and an epigenetic editor targeting CD3D/G-CRE. In some embodiments, an epigenetic editor targeting CIITA is used in combination with an epigenetic editor targeting TRAC and an epigenetic editor targeting CD247. In some embodiments, an epigenetic editor targeting CIITA is used in combination with an epigenetic editor targeting RFX5 and an epigenetic editor targeting CD3G. In some embodiments, an epigenetic editor targeting CIITA is used in combination with an epigenetic editor targeting RFX5 and an epigenetic editor targeting CD3D. In some embodiments, an epigenetic editor targeting CIITA is used in combination with an epigenetic editor targeting CD3D and an epigenetic editor targeting CD3G.

In some embodiments, four epigenetic editors are used in combination. In some embodiments, an epigenetic editor targeting B2M is used in combination with an epigenetic editor targeting CIITA, an epigenetic editor targeting TRAC, and an epigenetic editor targeting RFX5 or RFXAP. In some embodiments, an epigenetic editor targeting B2M is used in combination with an epigenetic editor targeting CIITA, an epigenetic editor targeting TRAC, and an epigenetic editor targeting CD247. In some embodiments, an epigenetic editor targeting B2M is used in combination with an epigenetic editor targeting CIITA, an epigenetic editor targeting TRAC, and an epigenetic editor targeting CD3D. In some embodiments, an epigenetic editor targeting B2M is used in combination with an epigenetic editor targeting CIITA, an epigenetic editor targeting TRAC, and an epigenetic editor targeting CD3G. In some embodiments, an epigenetic editor targeting B2M is used in combination with an epigenetic editor targeting CIITA, an epigenetic editor targeting TRAC, and an epigenetic editor targeting CD3D/G-CRE.

In some embodiments, five or more epigenetic editors are used in combination. In some embodiments, an epigenetic editor targeting B2M is used in combination with an epigenetic editor targeting CIITA, an epigenetic editor targeting TRAC, an epigenetic editor targeting RFX5 or RFXAP and an epigenetic editor targeting CD3D. In some embodiments, an epigenetic editor targeting B2M is used in combination with an epigenetic editor targeting CIITA, an epigenetic editor targeting TRAC, an epigenetic editor targeting CD3G, and an epigenetic editor targeting CD3D. In some embodiments, an epigenetic editor targeting B2M is used in combination with an epigenetic editor targeting CIITA, an epigenetic editor targeting TRAC, an epigenetic editor targeting CD247, and an epigenetic editor targeting CD3D. In some embodiments, an epigenetic editor targeting B2M is used in combination with an epigenetic editor targeting CIITA, an epigenetic editor targeting CD247, an epigenetic editor targeting RFX5 or RFXAP, and an epigenetic editor targeting CD3D.

The HLA-E fusion protein consists of the HLA-E heavy chain covalently fused to B2M and to the leader sequence derived from an HLA class I allele. The HLA-E fusion protein can interact with CD94/NKG2A or CD94/NKG2C heterodimers expressed on the surface of NK cells and result in inhibition (NKG2A) of or activation of NK cytotoxicity (NKG2C). Any epigenetic editor and multiplexing approach of the present disclosure can be combined with delivery of an HLA class I histocompatibility antigen, alpha chain E (HLA-E) fusion protein, including variant sequences, to achieve an NK “shield”, minimizing activation through NKG2C while providing protection from natural killer cells. In some embodiments, the variants comprise the sequence VMAPRTLIL (SEQ ID NO: 674), VMAPRTLLL (SEQ ID NO: 675), VMAPRTLVL (SEQ ID NO: 676), VMAPRTVLL (SEQ ID NO: 677), and/or VMAPRTLFL (SEQ ID NO: 678).

### VI. TARGET SEQUENCES

An epigenetic editor herein may be directed to a target sequence to effect epigenetic modification of a target gene.

As used herein, a “target sequence,” a “target site,” or a “target region” is a nucleic acid sequence present in a gene of interest; in some instances, the target sequence may be outside but in the vicinity of the gene of interest wherein methylation or binding by a repressor of the target sequence represses expression of the gene. In some embodiments, the target sequence may be a hypomethylated or hypermethylated nucleic acid sequence.

The target sequence may be in any part of a target gene. In some embodiments, the target sequence is part of or near a noncoding sequence of the gene. In some embodiments, the target sequence is part of an exon of the gene. In some embodiments, the target sequence is part of or near a transcriptional regulatory sequence of the gene, such as a promoter or an enhancer. In some embodiments, the target sequence is adjacent to, overlaps with, or encompasses a CpG island. In certain embodiments, the target sequence is within about 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, or 100 base pairs (bp) flanking a target gene TSS. In certain embodiments, the target sequence is within 500 bp flanking the target gene TSS. In certain embodiments, the target sequence is within 1000 bp flanking the target gene TSS.

In some embodiments, the target sequence may hybridize to a guide polynucleotide sequence (e.g., gRNA) complexed with a fusion protein comprising a polynucleotide guided DNA-binding domain (e.g., a CRISPR protein such as dCas9) and effector domain(s). The guide polynucleotide sequence may be designed to have complementarity to the target sequence, or identity to the opposing strand of the target sequence. In some embodiments, the guide polynucleotide comprises a spacer sequence that is about 80%, 85%, 90%, 91%, 92%, 93%, 94%, 95%, 96%, 97%, 98% or 99% identical to a protospacer sequence in the target sequence. In particular embodiments, the guide polynucleotide comprises a spacer sequence that is 100% identical to a protospacer sequence in the target sequence.

In some embodiments, where the DNA-binding domain of an epigenetic editor described herein is a zinc finger array, the target sequence may be recognized by said zinc finger array.

In some embodiments, where the DNA-binding domain of an epigenetic editor described herein is a TALE, the target sequence may be recognized by said TALE. A target sequence described herein may be specific to one copy of a target gene, or may be specific to one allele of a target gene. Accordingly, the epigenetic modification and modulation of expression thereof may be specific to one copy or one allele of the target gene. For example, an epigenetic editor may repress expression of a specific copy harboring a target sequence recognized by the DNA-binding domain (e.g., a copy associated with a disease or condition, or that harbors a mutation associated with a disease or condition). In some embodiments, the target genomic region may fall within the sequence shown in SEQ ID NO: 1219, 1220, 1283, 1284, 1285, 1286, 1287, 1288, 1313, or 1314.

### VII. EPIGENETIC MODIFICATIONS

An epigenetic editor described herein may perform sequence-specific epigenetic modification(s) (e.g., alteration of chemical modification(s)) of a target gene that harbors the target sequence. Such epigenetic modulation may be safer and more easily reversible than modulation due to gene editing, e.g., with generation of DNA double-strand breaks. In some embodiments, the epigenetic modulation may reduce or silence the target gene. In some embodiments, the modification is at a specific site of the target sequence. In some embodiments, the modification is at a specific allele of the target gene. Accordingly, the epigenetic modification may result in modulated (e.g., reduced) expression of one copy of a target gene harboring a specific allele, and not the other copy of the target gene. In some embodiments, the specific allele is associated with a disease, condition, or disorder.

In some embodiments, the epigenetic modification reduces or abolishes transcription of the target gene harboring the target sequence. In some embodiments, the epigenetic modification reduces or abolishes transcription of a copy of the target gene harboring a specific allele recognized by the epigenetic editor. In some embodiments, the epigenetic editor reduces the level of or eliminates expression of a protein encoded by the target gene. In some embodiments, the epigenetic editor reduces the level of or eliminates expression of a protein encoded by a copy of the target gene harboring a specific allele recognized by the epigenetic editor. The target gene may be epigenetically modified in vitro, ex vivo, or in vivo.

The effector domain of an epigenetic editor described herein may alter (e.g., deposit or remove) a chemical modification at a nucleotide of the target gene or at a histone associated with the target gene. The chemical modification may be altered at a single nucleotide or a single histone, or may be altered at 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 1500, 2000, 2500, 3000 or more nucleotides.

In some embodiments, an effector domain of an epigenetic editor described herein may alter a CpG dinucleotide within the target gene. In some embodiments, all CpG dinucleotides within 2000, 1500, 1000, 500, or 200 bps flanking a target sequence (e.g., in an alteration site as described herein) are altered according to a modification type described herein, as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more of the CpG dinucleotides are altered as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the CpG dinucleotides are altered as compared to the original state of the gene or the gene in a comparable cell not contacted with the epigenetic editor. In some embodiments, one single CpG dinucleotide is altered, as compared to the original state of the gene or the gene in a comparable cell not contacted with the epigenetic editor.

An effector domain of an epigenetic editor described herein may alter a histone modification state of a histone associated with or bound to the target gene. For example, an effector domain may deposit a modification on one or more lysine residues of histone tails of histones associated with the target gene. In some embodiments, the effector domain may result in deacetylation of one or more histone tails of histones associated with the target gene, thereby reducing or silencing expression of the target gene. In some embodiments, the histone modification state is a methylation state. For example, the effector domain may result in a H3K9, H3K27 or H4K20 methylation (e.g., one or more of a H3K9me2, H3K9me3, H3K27me2, H3K27me3, and H4K20me3 methylation) at one or more histone tails associated with the target gene, thereby reducing or silencing expression of the target gene.

In some embodiments, all histone tails of histones bound to DNA nucleotides within 2000, 1500, 1000, 500, or 200 bps flanking the target sequence are altered according to a modification type as described herein, as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of the bound histones are altered as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of the bound histones are altered as compared to the original state of the chromosome or the chromosome in a comparable cell not contacted with the epigenetic editor. For example, one single histone tail of the bound histones may be altered as compared to the original state of the chromosome or the chromosome in a comparable cell not contacted with the epigenetic editor. As another example, one single bound histone octamer may be altered as compared to the original state of the chromosome or the chromosome in a comparable cell not contacted with the epigenetic editor.

The chemical modification deposited at target gene DNA nucleotides or histone residues may be at or in close proximity to a target sequence in the target gene. In some embodiments, an effector domain of an epigenetic editor described herein alters a chemical modification state of a nucleotide or histone tail bound to a nucleotide 100-200, 200-300, 300-400, 400-55, 500-600, 600-700, or 700-800 nucleotides 5′ or 3′ to the target sequence in the target gene. In some embodiments, an effector domain alters a chemical modification state of a nucleotide or histone tail bound to a nucleotide within 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, or 2000 nucleotides flanking the target sequence. As used herein, “flanking” refers to nucleotide positions 5′ to the 5′ end of and 3′ to the 3′ end of a particular sequence, e.g., a target sequence.

In some embodiments, an effector domain mediates or induces a chemical modification change of a nucleotide or a histone tail bound to a nucleotide distant from a target sequence. Such modification may be initiated near the target sequence, and may subsequently spread to one or more nucleotides in the target gene distant from the target sequence. For example, an effector domain may initiate alteration of a chemical modification state of one or more nucleotides or one or more histone residues bound to one or more nucleotides within 10, 20, 30, 40, 50, 60, 70, 80, 90, 100, 200, 300, 400, 500 nucleotides flanking the target sequence, and the chemical modification state alteration may spread to one or more nucleotides at least 100, 200, 300, 400, 500, 600, 700, 800, 900, 1000, 1100, 1200, 1300, 1400, 1500, 1600, 1700, 1800, 1900, 2000, 2500, 3000, or more nucleotides from the target sequence in the target gene, either upstream or downstream of the target sequence. In certain embodiments, the chemical modification may be initiated at less than 2, 3, 5, 10, 20, 30, 40, 50, or 100 nucleotides in the target gene and spread to at least 100, 200, 300, 400, 500, 600, 700, 800, 900, 1000, 2000, or more nucleotides in the target gene. In some embodiments, the chemical modification spreads to nucleotides in the entire target gene. Additional proteins or transcription factors, for example, transcription repressors, methyltransferases, or transcription regulation scaffold proteins, may be involved in the spreading of the chemical modification. Alternatively, the epigenetic editor alone may be involved.

In some embodiments, an epigenetic editor described herein reduces expression of a target gene by at least about 20%, at least about 30%, at least about 40%, at least about 50%, at least about 60%, at least about 70%, at least about 80%, at least about 90%, at least about 95%, at least about 99%, or more, as measured by transcription of the target gene in a cell, a tissue, or a subject as compared to a control cell, control tissue, or a control subject (e.g., in the absence of the epigenetic editor). In some embodiments, the epigenetic editors described herein reduces expression of a copy of target gene by at least about 20%, at least about 30%, at least about 40%, at least about 50%, at least about 60%, at least about 70%, at least about 80%, at least about 90%, at least about 95%, at least about 99%, or more, as measured by transcription of the copy of the target gene in a cell, a tissue, or a subject as compared to a control cell, control tissue, or a control subject. In certain embodiments, the copy of the target gene harbors a specific sequence or allele recognized by the epigenetic editor. In particular embodiments, the epigenetically modified copy encodes a functional protein, and accordingly an epigenetic editor disclosed herein may reduce or abolish expression and/or function of the protein. For example, an epigenetic editor described herein may reduce expression and/or function of a protein encoded by the target gene by at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold, at least 60-fold, at least 70-fold, at least 80-fold, at least 90-fold, or at least 100 fold in a cell, a tissue, or a subject as compared to a control cell, control tissue, or a control subject.

Modulation of target gene expression can be assayed by determining any parameter that is indirectly or directly affected by the expression of the target gene. Such parameters include, e.g., changes in RNA or protein levels; changes in protein activity; changes in product levels; changes in downstream gene expression; changes in transcription or activity of reporter genes such as, for example, luciferase, CAT, beta-galactosidase, or GFP; changes in signal transduction; changes in phosphorylation and dephosphorylation; changes in receptor-ligand interactions; changes in concentrations of second messengers such as, for example, cGMP, CAMP, IP3, and Ca2+; changes in cell growth; changes in neovascularization; and/or changes in any functional effect of gene expression. Measurements can be made in vitro, in vivo, and/or ex vivo, and can be made by conventional methods, e.g., measurement of RNA or protein levels, measurement of RNA stability, and/or identification of downstream or reporter gene expression. Readout can be by way of, for example, chemiluminescence, fluorescence, colorimetric reactions, antibody binding, inducible markers, ligand binding assays, changes in intracellular second messengers such as cGMP and inositol triphosphate (IP3), changes in intracellular calcium levels; cytokine release, and the like.

Methods for determining the expression level of a gene, for example the target of an epigenetic editor, may include, e.g., determining the transcript level of a gene by reverse transcription PCR, quantitative RT-PCR, droplet digital PCR (ddPCR), Northern blot, RNA sequencing, DNA sequencing (e.g., sequencing of complementary deoxyribonucleic acid (cDNA) obtained from RNA); next generation (Next-Gen) sequencing, nanopore sequencing, pyrosequencing, or Nanostring sequencing. Levels of protein expressed from a gene may be determined, e.g., by Western blotting, enzyme linked immuno-absorbance assays, mass-spectrometry, immunohistochemistry, or flow cytometry analysis. Gene expression product levels may be normalized to an internal standard such as total messenger ribonucleic acid (mRNA) or the expression level of a particular gene, e.g., a housekeeping gene.

In some embodiments, the effect of an epigenetic editor in modulating target gene expression may be examined using a reporter system. For example, an epigenetic editor may be designed to target a reporter gene encoding a reporter protein, such as a fluorescent protein. Expression of the reporter gene in such a model system may be monitored by, e.g., flow cytometry, fluorescence-activated cell sorting (FACS), or fluorescence microscopy. In some embodiments, a population of cells may be transfected with a vector that harbors a reporter gene. The vector may be constructed such that the reporter gene is expressed when the vector transfects a cell. Suitable reporter genes include genes encoding fluorescent proteins, for example green, yellow, cherry, cyan or orange fluorescent proteins. The population of cells carrying the reporter system may be transfected with DNA, mRNA, or vectors encoding the epigenetic editor targeting the reporter gene.

### VIII. EPIGENETICALLY MODIFIED CELLS

In one aspect, the present disclosure provides cells that have been modified using one or more epigenetic editor(s) described herein. In some embodiments, nucleic acid molecule(s) encoding said epigenetic editor(s) or component(s) thereof are administered to the cells. Any type of cell may be modified as described herein. The cells may be modified in vitro, in vivo, or ex vivo. Cells suitable for modification may be procured from a patient or a healthy donor.

In some embodiments, the cell is an immune cell. Immune cells may include T cells, B cells, natural killer (NK) cells, dendritic cells, and monocytes/macrophages. In some embodiments, the cell is an alpha/beta T cell. In some embodiments, the cell is a gamma/delta T cell. In some embodiments, the cell is a cytotoxic T cell, e.g., a CD8+ cytotoxic T cell. In some embodiments, the cell is a T helper cell, e.g., a CD4+ T helper cell. In some embodiments, the cell is a regulatory T cell. In some embodiments, the cell is an NK cell. In some embodiments, the cell is a dendritic cell. In some embodiments, the cell is a macrophage.

In some embodiments, the cell is a stem cell. A “stem cell” refers to an undifferentiated cell which is capable of indefinitely giving rise to more stem cells of the same type, and from which other specialized cells may arise by differentiation. Adult stem cells are usually multipotent, while induced or embryonic-derived stem cells are pluripotent. In some embodiments, the cell is a progenitor cell. A “progenitor cell” refers to a cell which is able to differentiate to form one or more types of cells, but has limited self-renewal in vitro and in vivo.

In some embodiments, the cell is capable of differentiating into an immune cell described above. The cell may be, for example, an embryonic stem cell (ESC), a hematopoietic stem cell (HSC), a hematopoietic progenitor cell (HPC), or a hematopoietic stem and progenitor cell (HSPC). A “hematopoietic stem and progenitor cell” or “HSPC” refers to a cell which expresses the antigenic marker CD34 (CD34+). In particular embodiments, the term “HSPC” refers to a cell identified by the presence of the antigenic marker CD34 (CD34+) and the absence of lineage (lin) markers. The population of cells that are CD34+ and/or Lin-includes hematopoietic stem cells and hematopoietic progenitor cells. In some embodiments, the cell is an induced pluripotent stem cell (iPSC) reprogrammed from a somatic cell such as a T cell.

In some embodiments, the cell is obtained from umbilical cord blood of a healthy donor. In some embodiments, the cell is obtained from adult peripheral blood or mobilized from the bone marrow of a healthy donor.

In some embodiments, a cell as described above is modified by a method comprising transfecting the cell with a system comprising (a) one or more epigenetic editor(s) described herein, or (b) nucleic acid molecule(s) encoding said epigenetic editor(s). In certain embodiments, the modified cell is a T cell. In some embodiments, the modified T cell expresses one or more epigenetic editor(s) that are able to selectively reduce or silence the expression of one or more target gene(s) in the cell. In particular embodiments, the target gene is B2M, CIITA, TRAC, CD247, RFX5, RFXAP, CD3D, or CD3G. In some embodiments, the T cells are modified ex vivo. The modified T cell may, in some embodiments, further express an engineered TCR or CAR directed against at least one antigen expressed at the surface of a target cell (e.g., a malignant or infected cell). In some embodiments, the modified T cell does not express at least one gene encoding an endogenous TCR component. In particular embodiments, the modified T cells are non-alloreactive. In particular embodiments, the modified T cells are particularly suitable for allogeneic transplantation.

### IX. PHARMACEUTICAL COMPOSITIONS

In one aspect, the present disclosure provides a pharmaceutical composition comprising as an active ingredient (or as the sole active ingredient) one or more epigenetic editors described herein or component(s) (e.g., fusion proteins and/or guide polynucleotides) thereof, or nucleic acid molecule(s) encoding said epigenetic editors or component(s) thereof. For example, a pharmaceutical composition may comprise nucleic acid molecule(s) encoding the fusion protein(s) (and guide polynucleotides, where applicable) of an epigenetic editor described herein. In some embodiments, separate pharmaceutical compositions comprise the fusion protein(s) and the guide polynucleotide(s).

In one aspect, the present disclosure provides a pharmaceutical composition comprising as an active ingredient (or as the sole active ingredient) cells that have undergone epigenetic modification(s) mediated or induced by (a) one or more epigenetic editor(s) provided herein, e.g., wherein nucleic acid molecule(s) encoding said epigenetic editor(s) were administered to said cells ex vivo.

Generally, the epigenetic editors described herein or component(s) thereof, nucleic acid molecule(s) encoding said epigenetic editors or component(s) thereof, or cells modified by the epigenetic editors of the present disclosure, are suitable to be administered as a formulation in association with one or more pharmaceutically acceptable excipient(s), e.g., as described below.

The term “excipient” is used herein to describe any ingredient other than the compound(s) of the present disclosure. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives, or buffers, which enhance the shelf life or effectiveness of the antibody.

Formulations of a pharmaceutical composition suitable for parenteral administration typically comprise the active ingredient combined with a pharmaceutically acceptable carrier, such as sterile water or sterile isotonic saline. Such formulations may be prepared, packaged, or sold in a form suitable for bolus administration or for continuous administration. The pharmaceutical compositions described herein may be administered to a subject, e.g., subcutaneously, intradermally, intratumorally, intranodally, intramuscularly, intravenously, intralymphatically, or intraperitoneally. In particular embodiments, a pharmaceutical composition of the present disclosure is administered intravenously to the subject.

### X. DELIVERY METHODS

In some embodiments, the epigenetic editor or its component(s) are introduced to target cells in the form of nucleic acid molecule(s) encoding the epigenetic editor or its component(s); accordingly, the pharmaceutical compositions herein comprise the nucleic acid molecule(s). Such nucleic acid molecule(s) may be, for example, DNA, RNA, or mRNA, and/or modified nucleic acid sequence(s) (e.g., with chemical modifications, a 5′ cap, or one or more 3′ modifications). In some embodiments, the nucleic acid molecule(s) may be delivered as naked DNA or RNA, for instance by means of transfection or electroporation, or can be conjugated to molecules (e.g., N-acetylgalactosamine) promoting uptake by target cells. In some embodiments, the nucleic acid molecule(s) may be in nucleic acid expression vector(s), which may include expression control sequences such as promoters, enhancers, transcription signal sequences, transcription termination sequences, introns, polyadenylation signals, Kozak consensus sequences, internal ribosome entry sites (IRES), etc. Such expression control sequences are well known in the art. A vector may also comprise a sequence encoding a signal peptide (e.g., for nuclear localization, nucleolar localization, or mitochondrial localization), associated with (e.g., inserted into or fused to) a sequence coding for a protein.

Examples of vectors include, but are not limited to, plasmid vectors; viral vectors based on vaccinia virus, poliovirus, adenovirus, adeno-associated virus, SV40, herpes simplex virus, human immunodeficiency virus, retrovirus (e.g., Murine Leukemia Virus, or spleen necrosis virus, vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and other recombinant vectors. In certain embodiments, the vector is a plasmid or a viral vector. Viral particles or virus-like particles (VLPs) may also be used to deliver nucleic acid molecule(s) encoding epigenetic editors or component(s) thereof as described herein. For example, “empty” viral particles can be assembled to contain any suitable cargo. Viral vectors and viral particles may also be engineered to incorporate targeting ligands to alter target tissue specificity.

In certain embodiments, an epigenetic editor as described herein or component(s) thereof are encoded by nucleic acid sequence(s) present in one or more viral vectors, or a suitable capsid protein of any viral vector. Examples of viral vectors include adeno-associated viral vectors (e.g., derived from AAV3, AAV3b, AAV4, AAV5, AAV6, AAV7, AAV8, AAV9, AAVrh8, AAV10, and/or variants thereof); retroviral vectors (e.g., Maloney murine leukemia virus, MML-V), adenoviral vectors (e.g., AD100), lentiviral vectors (e.g., HIV and FIV-based vectors), and herpesvirus vectors (e.g., HSV-2).

In some embodiments, delivery involves an adeno-associated virus (AAV) vector. AAV vector delivery may be particularly useful where the DNA-binding domain of an epigenetic editor fusion protein is a zinc finger array. Without wishing to be bound by any theory, the smaller size of zinc finger arrays compared to larger DNA-binding domains such as Cas protein domains may allow such a fusion protein to be conveniently packed in viral vectors such as an AAV vector.

Any AAV serotype, e.g., human AAV serotype, can be used for an AAV vector as described herein, including, but not limited to, AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), and AAV serotype 11 (AAV11), as well as variants thereof. In some embodiments, an AAV variant has at least 90%, 91%, 92%, 93%, 94%, 95%, 96%, 97%, 98%, or 99% amino acid sequence identity to a wildtype AAV. In certain embodiments, the AAV variant may be engineered such that its capsid proteins have reduced immunogenicity or enhanced transduction ability in humans. In some instances, one or more regions of at least two different AAV serotype viruses are shuffled and reassembled to generate a chimeric variant. For example, a chimeric AAV may comprise inverted terminal repeats (ITRs) that are of a heterologous serotype compared to the serotype of the capsid. The resulting chimeric AAV can have a different antigenic reactivity or recognition compared to its parental serotypes. In some embodiments, a chimeric variant of an AAV includes amino acid sequences from 2, 3, 4, 5, or more different AAV serotypes.

Non-viral systems are also contemplated for delivery as described herein. Non-viral systems include, but are not limited to, nucleic acid transfection methods including electroporation, sonoporation, calcium phosphate transfection, microinjection, DNA biolistics, lipid-mediated transfection, transfection through heat shock, compacted DNA-mediated transfection, lipofection, cationic agent-mediated transfection, and transfection with liposomes, immunoliposomes, exosomes, or cationic facial amphiphiles (CFAs). In certain embodiments, one or more mRNAs encoding epigenetic editor fusion proteins as described herein may be co-electroporated with one or more guide polynucleotides (e.g., gRNAs) as described herein. One important category of non-viral nucleic acid vectors is nanoparticles, which can be organic (e.g., lipid) or inorganic (e.g., gold). For instance, organic (e.g. lipid and/or polymer) nanoparticles can be suitable for use as delivery vehicles in certain embodiments of this disclosure.

In some embodiments, delivery is accomplished using a lipid nanoparticle (LNP). LNP compositions are typically sized on the order of micrometers or smaller and may include a lipid bilayer. In some embodiments, an LNP refers to any particle that has a diameter of less than 1000 nm, 500 nm, 250 nm, 200 nm, 150 nm, 100 nm, 75 nm, 50 nm, or 25 nm. In some embodiments, a nanoparticle may range in size from 1-1000 nm, 1-500 nm, 1-250 nm, 25-200 nm, 25-100 nm, 35-75 nm, or 25-60 nm. Nanoparticle compositions encompass lipid nanoparticles (LNPs), liposomes (e.g., lipid vesicles), and lipoplexes.

An LNP as described herein may be made from cationic, anionic, or neutral lipids. In some embodiments, an LNP may comprise neutral lipids, such as the fusogenic phospholipid 1,2-Dioleoyl-sn-glycero-3-phosphoethanolamine (DOPE) or the membrane component cholesterol, as helper lipids to enhance transfection activity and nanoparticle stability. In some embodiments, an LNP may comprise hydrophobic lipids, hydrophilic lipids, or both hydrophobic and hydrophilic lipids. Any lipid or combination of lipids that are known in the art can be used to produce an LNP. The lipids may be combined in any molar ratios to produce the LNP. In some embodiments, the LNP is a T cell-targeting (e.g., preferentially or specifically targeting the T cell) LNP.

### XI. THERAPEUTIC USES OF EPIGENETIC EDITORS AND MODIFIED CELLS

The present disclosure also provides methods for treating or preventing a condition in a subject, comprising administering to the subject a) one or more epigenetic editor(s) as described herein, b) nucleic acid molecule(s) encoding the epigenetic editor(s), c) cells modified by the epigenetic editor(s), or d) pharmaceutical compositions comprising any of a)-c).

In one aspect, the epigenetic editor may effect an epigenetic modification of a target polynucleotide sequence in a target gene associated with a disease, condition, or disorder in the subject, thereby modulating expression of the target gene to treat or prevent the disease, condition, or disorder. In some embodiments, the epigenetic editor reduces the expression of the target gene to an extent sufficient to achieve a desired effect, e.g., a therapeutically relevant effect such as the prevention or treatment of the disease, condition, or disorder.

In one aspect, a cell (e.g., an allogeneic cell) modified by one or more epigenetic editor(s) of the present disclosure may be administered as a medicament to a subject with a disease, condition, or disorder, thereby treating the disease, condition, or disorder. In some embodiments, the subject is administered allogeneic T cells which have been epigenetically modified as described herein, e.g., to have reduced or silenced target gene expression. In some embodiments, the modified T cells further express an engineered TCR or CAR directed against at least one antigen expressed at the surface of a target cell (e.g., a malignant or infected cell). In some embodiments, the modified T cells do not express at least one gene encoding an endogenous TCR component.

In some embodiments, the subject may be a mammal, e.g., a human. In some embodiments, the subject is selected from a non-human primate such as chimpanzee, cynomolgus monkey, or macaque, and other ape and monkey species.

### XII. DEFINITIONS

The term “nucleic acid” as used herein refers to any oligonucleotide or polynucleotide containing nucleotides (e.g., deoxyribonucleotides or ribonucleotides) in either single- or double-strand form, and includes DNA and RNA. “Nucleotides” contain a sugar deoxyribose (DNA) or ribose (RNA), a base, and a phosphate group, and are linked together through the phosphate groups. “Bases” include purines and pyrimidines, which include natural compounds such as adenine, thymine, guanine, cytosine, uracil, inosine, and natural analogs; as well as synthetic derivatives of purines and pyrimidines, which include, but are not limited to, modified versions which place new reactive groups such as amines, alcohols, thiols, carboxylates, alkylhalides, etc. Nucleic acids may contain known nucleotide analogs and/or modified backbone residues or linkages, which may be synthetic, naturally occurring, and non-naturally occurring. Such nucleotide analogs, modified residues, and modified linkages are well known in the art, and may provide a nucleic acid molecule with enhanced cellular uptake, reduced immunogenicity, and/or increased stability in the presence of nucleases.

As used herein, an “isolated” or “purified” nucleic acid molecule is a nucleic acid molecule that exists apart from its native environment. For example, an “isolated” or “purified” nucleic acid molecule (1) has been separated away from the nucleic acids of the genomic DNA or cellular RNA of its source of origin; and/or (2) does not occur in nature. In some embodiments, an “isolated” or “purified” nucleic acid molecule is a recombinant nucleic acid molecule.

It will be understood that in addition to the specific proteins and nucleic acid molecules mentioned herein, the present disclosure also contemplates the use of variants, derivatives, homologs, and fragments thereof. A variant of any given sequence may have the specific sequence of residues (whether amino acid or nucleic acid residues) modified in such a manner that the polypeptide or polynucleotide in question substantially retains at least one of its endogenous functions. A variant sequence can be obtained by addition, deletion, substitution, modification, replacement and/or variation of at least one residue present in the naturally-occurring sequence (in some embodiments, no more than 1, 2, 3, 4, 5, 6, 7, 8, 9, 10, 15, or 20 residues). For specific proteins described herein (e.g., KRAB, dCas9, DNMT3A, and DNMT3L proteins described herein), the present disclosure also contemplates any of the protein's naturally occurring forms, or variants or homologs that retain at least one of its endogenous functions (e.g., at least 50%, 60%, 70%, 80%, 90%, 85%, 96%, 97%, 98%, or 99% of its function as compared to the specific protein described).

As used herein, a homologue of any polypeptide or nucleic acid sequence contemplated herein includes sequences having a certain homology with the wildtype amino acid and nucleic sequence. A homologous sequence may include a sequence, e.g., an amino acid sequence which may be at least 50%, 55%, 65%, 75%, 85%, 90%, 91%, 92%<93%, 94%, 95%, 96%, 97%, 98%, or 99% identical to the subject sequence. The term “percent identical” in the context of amino acid or nucleotide sequences refers to the percent of residues in two sequences that are the same when aligned for maximum correspondence. In some embodiments, the length of a reference sequence aligned for comparison purposes is at least 30%, (e.g., at least 40, 50, 60, 70, 80, or 90%, or 100%) of the reference sequence.

Sequence identity may be measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e-3 and e-100 indicating a closely related sequence.

The percent identity of two nucleotide or polypeptide sequences is determined by, e.g., BLAST® using default parameters (available at the U.S. National Library of Medicine's National Center for Biotechnology Information website). In some embodiments, the length of a reference sequence aligned for comparison purposes is at least 30%, (e.g., at least 40, 50, 60, 70, 80, or 90%) of the reference sequence.

It will be understood that the numbering of the specific positions or residues in polypeptide sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

The term “modulate” or “alter” refers to a change in the quantity, degree, or extent of a function. For example, an epigenetic editor as described herein may modulate the activity of a promoter sequence by binding to a motif within the promoter, thereby inducing, enhancing, or suppressing transcription of a gene operatively linked to the promoter sequence. As other examples, an epigenetic editor as described herein may block RNA polymerase from transcribing a gene, or may inhibit translation of an mRNA transcript. The terms “inhibit,” “repress,” “suppress,” “silence” and the like, when used in reference to an epigenetic editor or a component thereof as described herein, refers to decreasing or preventing the activity (e.g., transcription) of a nucleic acid sequence (e.g., a target gene) or protein relative to the activity of the nucleic acid sequence or protein in the absence of the epigenetic editor or component thereof. The term may include partially or totally blocking activity, or preventing or delaying activity. The inhibited activity may be, e.g., 10%, 20%, 30%, 40%, 50%, 60%, 70%, 75%, 80%, 85%, 90%, 91%, 92%, 93%, 94%, 95%, 96%, 97%, 98%, or 99% less than that of a control, or may be, e.g., at least 1.5-fold, 2-fold, 3-fold, 4-fold, 5-fold, or 10-fold less than that of a control.

The term “about” or “approximately” means within an acceptable error range for the particular value as determined by one of ordinary skill in the art, which will depend in part on how the value is measured or determined, e.g., the limitations of the measurement system. For example, “about” can mean within one or more than one standard deviation, per the practice in the given value. Where particular values are described in the application and claims, unless otherwise stated, the term “about” should be assumed to mean an acceptable error range for the particular value.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. Unless otherwise indicated, the recitation of a listing of elements herein includes any of the elements singly or in any combination. The recitation of an embodiment herein includes that embodiment as a single embodiment, or in combination with any other embodiment(s) herein. All publications, patents, patent applications, and other references mentioned herein are incorporated by reference in their entirety. To the extent that references incorporated by reference contradict the disclosure contained in the specification, the specification is intended to supersede and/or take precedence over any such contradictory material. Although a number of documents are cited herein, this citation does not constitute an admission that any of these documents forms part of the common general knowledge in the art.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that the present disclosure may be better understood, the following examples are set forth. These examples are for purposes of illustration only and are not to be construed as limiting the scope of the present disclosure in any manner.

### EXAMPLES

### Example 1: Fusion Protein Design and Synthesis

A fusion protein comprising dCas9, DNMT3A, DNMT3L, and KOX1 KRAB (“CRISPR-off”) was produced. From N terminus to C terminus, the protein had the following functional domains and linkers: huDNMT3A-linker-huDNMT3L-XTEN80-NLS-dSpCas9-NLS-XTEN16-huKOX1 KRAB (SEQ ID NO: 658). The CRISPR-off plasmid construct is described in Nuñez et al., Cell (2021) 184 (9): 2503-19.

ZF fusion proteins (“ZF-off”) comprising DNMT3A, 3L, and KOX1 KRAB were also produced. These fusion proteins had the following general structure: huDNMT3A-linker-huDNMT3L-XTEN80-NLS-ZFP domain-NLS-XTEN16-huKOX1 KRAB (SEQ ID NO: 659).

### Example 2: Selection of Regions for gRNA Targeting

gRNAs targeting genomic regions within 1 kb of the TSS of a human B2M, CIITA, TRAC, CD247, RFX5, CD3D, or CD3G gene or a CD3D/G-CRE region were computationally designed using the Benchling gRNA platform for human (GRCh38). gRNAs containing poly-TTTT sequences were first discarded. gRNA off-target analysis using CasOFFinder (Bae et al., Bioinformatics (2014) 30 (10): 1473-5) was performed. gRNAs were discarded if they matched to multiple locations across the target genome.

Exemplary gRNA sequences are shown in Tables 4-11.

gRNAs were generated to target the sequences in Tables 4-11 by standard methods using a tracr sequence from Table 12 and modifications as described below:

(SEQ ID NO: 2081)
mN*mN*mN*rNrNrNrNrNrNrNrNrNrNrNrNrNrNrNrNrNrGrUrUr
UrUrArGrAmGmCmUmAmGmAmAmAmUmAmGmCrArArGrUrUrArArAr
ArUrArArGrGrCrUrArGrUrCrCrGrUrUrArUrCrAmAmCmUmUmGm
AmAmAmAmAmGmUmGmGmCmAmCmCmGmAmGmUmCmGmGmUmGmCmU*mU
*mU*mU

where “N” indicates an RNA base in the SPACR/target region, “r” indicates ribose, “m” means 2′O-me ribose, and “*” indicates a phosphorothioate modification.

### Example 3: Multiplex Silencing of T-Cell Targets in Primary Human T Cells

This example describes a study in which combinations of gRNAs targeting B2M, CIITA, TRAC, CD247, RFX5, RFXAP, CD3D, or CD3G were used for multiplexed epigenetic gene silencing in primary human T cells.

The guide RNAs used in the multiplexing experiments described below are provided in Table 16 below:

TABLE 16
gRNAs used in multiplexing experiments

| | | | SEQ | | SEQ |
|---|---|---|---|---|---|
| | | Target Sequence | ID | | ID |
| gRNA | Target | (DNA) | NO | gRNA sequence | NO |
| RNA1 | B2M | GGCCACGGAGCGAGACA | 2082 | GGCCACGGAGCGAGACATCTG | 2089 |
| 04 | | TCT | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA1 | B2M | GCGCCCCAGCTTGGGACA | 2074 | GCGCCCCAGCTTGGGACACCG | 2090 |
| 10 | | CC | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA1 | B2M | GCGCCCCAGCTTGGGACA | 2074 | GCGCCCCAGCTTGGGACACCG | 2090 |
| 10 | | CC | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA1 | B2M | CGCAGTGCCAGGTTAGAG | 2083 | CGCAGTGCCAGGTTAGAGAGG | 2091 |
| 12 | | AG | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA1 | B2M | CGCAGTGCCAGGTTAGAG | 2083 | CGCAGTGCCAGGTTAGAGAGG | 2091 |
| 12 | | AG | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA1 | B2M | CGGCTCTGCTTCCCTTAG | 2084 | CGGCTCTGCTTCCCTTAGACGT | 2092 |
| 38 | | AC | | TTAAGAGCTAAGCTGGAAACA | |
| | | | | GCATAGCAAGTTTAAATAAGG | |
| | | | | CTAGTCCGTTATCAACTTGAA | |
| | | | | AAAGTGGCACCGAGTCGGTGC | |
| | | | | TTTTTT | |
| RNA1 | B2M | CGGCTCTGCTTCCCTTAG | 2084 | CGGCTCTGCTTCCCTTAGACGT | 2092 |
| 38 | | AC | | TTAAGAGCTAAGCTGGAAACA | |
| | | | | GCATAGCAAGTTTAAATAAGG | |
| | | | | CTAGTCCGTTATCAACTTGAA | |
| | | | | AAAGTGGCACCGAGTCGGTGC | |
| | | | | TTTTTT | |
| RNA9 | B2M | GCGCCCGGTGTCCCAAGC | 2085 | GCGCCCGGTGTCCCAAGCTGG | 2093 |
| 49 | | TG | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA9 | B2M | GCGCCCGGTGTCCCAAGC | 2085 | GCGCCCGGTGTCCCAAGCTGG | 2093 |
| 49 | | TG | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA1 | CD24 | CACAGTTGCCGATTACAG | 1628 | CACAGTTGCCGATTACAGGTG | 2094 |
| 307 | 7 | GT | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA1 | CD24 | AGGTGGCGAAGGCGCTG | 1457 | AGGTGGCGAAGGCGCTGTGGG | 2095 |
| 409 | 7 | TGG | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA1 | CD24 | GAGCAACCGAGGCAGGC | 1483 | GAGCAACCGAGGCAGGCCCA | 2096 |
| 460 | 7 | CCA | | GTTTAAGAGCTAAGCTGGAAA | |
| | | | | CAGCATAGCAAGTTTAAATAA | |
| | | | | GGCTAGTCCGTTATCAACTTG | |
| | | | | AAAAAGTGGCACCGAGTCGGT | |
| | | | | GCTTTTTT | |
| RNA1 | CD24 | TGCTCCGTCACAGCCGAG | 1542 | TGCTCCGTCACAGCCGAGTTG | 2097 |
| 473 | 7 | TT | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA1 | CD24 | CCACAGTCCTCCACTTCC | 1622 | CCACAGTCCTCCACTTCCTGGT | 2098 |
| 544 | 7 | TG | | TTAAGAGCTAAGCTGGAAACA | |
| | | | | GCATAGCAAGTTTAAATAAGG | |
| | | | | CTAGTCCGTTATCAACTTGAA | |
| | | | | AAAGTGGCACCGAGTCGGTGC | |
| | | | | TTTTTT | |
| RNA1 | CD24 | CCACAGTCCTCCACTTCC | 1622 | CCACAGTCCTCCACTTCCTGGT | 2098 |
| 544 | 7 | TG | | TTAAGAGCTAAGCTGGAAACA | |
| | | | | GCATAGCAAGTTTAAATAAGG | |
| | | | | CTAGTCCGTTATCAACTTGAA | |
| | | | | AAAGTGGCACCGAGTCGGTGC | |
| | | | | TTTTTT | |
| RNA1 | CD24 | AGGAAGCCCTGGAGGTTT | 1605 | AGGAAGCCCTGGAGGTTTGAG | 2099 |
| 566 | 7 | GA | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA2 | CD3D | TCTCACCCAGGCTGATAG | 2086 | TCTCACCCAGGCTGATAGTTG | 2100 |
| 329 | | TT | | TTTTAGAGCTAGAAATAGCAA | |
| | | | | GTTAAAATAAGGCTAGTCCGT | |
| | | | | TATCAACTTGAAAAAGTGGCA | |
| | | | | CCGAGTCGGTGCTTTT | |
| RNA2 | CD3G | GCTGGCTGGCTGGCTGCT | 2087 | GCTGGCTGGCTGGCTGCTAAG | 2101 |
| 510 | | AA | | TTTTAGAGCTAGAAATAGCAA | |
| | | | | GTTAAAATAAGGCTAGTCCGT | |
| | | | | TATCAACTTGAAAAAGTGGCA | |
| | | | | CCGAGTCGGTGCTTTT | |
| RNA2 | CIITA | ATTGGCAGCTGGCACCAG | 928 | ATTGGCAGCTGGCACCAGTGG | 2102 |
| 32 | | TG | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA2 | CIITA | CCCTGCAAGGTGGCCCCA | 963 | CCCTGCAAGGTGGCCCCAAGG | 2103 |
| 58 | | AG | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA2 | CIITA | CCCTGCAAGGTGGCCCCA | 963 | CCCTGCAAGGTGGCCCCAAGG | 2103 |
| 58 | | AG | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA2 | CIITA | TAGCTTGGCGGGCTCCCA | 985 | TAGCTTGGCGGGCTCCCAACG | 2104 |
| 68 | | AC | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA2 | CIITA | GCGGGCTCCCAACTGGTG | 996 | GCGGGCTCCCAACTGGTGACG | 2105 |
| 69 | | AC | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA2 | CIITA | GCGGGCTCCCAACTGGTG | 996 | GCGGGCTCCCAACTGGTGACG | 2105 |
| 69 | | AC | | TTTAAGAGCTAAGCTGGAAAC | |
| | | | | AGCATAGCAAGTTTAAATAAG | |
| | | | | GCTAGTCCGTTATCAACTTGA | |
| | | | | AAAAGTGGCACCGAGTCGGTG | |
| | | | | CTTTTTT | |
| RNA4 | CIITA | GGCTTATGCCAATATCGG | 2088 | GGCTTATGCCAATATCGGTGG | 2106 |
| 64 | | TG | | TTTAAGAGCTAGAAATAGCAA | |
| | | | | GTTTAAATAAGGCTAGTCCGT | |
| | | | | TATCAACTTGAAAAAGTGGCA | |
| | | | | CCGAGTCGGTGCTTTTTT | |
| RNA2 | RFX5 | CCGGCTGGGCCAGGACCT | 1653 | CCGGCTGGGCCAGGACCTTGG | 2107 |
| 628 | | TG | | TTTTAGAGCTAGAAATAGCAA | |
| | | | | GTTAAAATAAGGCTAGTCCGT | |
| | | | | TATCAACTTGAAAAAGTGGCA | |
| | | | | CCGAGTCGGTGCTTTT | |
| RNA2 | RFX5 | GCAGTGACCAATGACAG | 1689 | GCAGTGACCAATGACAGAGCG | 2108 |
| 668 | | AGC | | TTTTAGAGCTAGAAATAGCAA | |
| | | | | GTTAAAATAAGGCTAGTCCGT | |
| | | | | TATCAACTTGAAAAAGTGGCA | |
| | | | | CCGAGTCGGTGCTTTT | |
| RNA2 | RFXA | GTCAGCGTGTGTACGCCC | 1904 | GTCAGCGTGTGTACGCCCGCG | 2109 |
| 977 | P | GC | | TTTTAGAGCTAGAAATAGCAA | |
| | | | | GTTAAAATAAGGCTAGTCCGT | |
| | | | | TATCAACTTGAAAAAGTGGCA | |
| | | | | CCGAGTCGGTGCTTTT | |

The respective gRNAs were generated standard methods using a tracr sequence as indicated in Table 12, and with the three 5′-terminal nucleotides and the three 3′-terminal nucleotides modified with 2′-O-Me and phosphorothioate modifications.

The epigenetic editor used in these experiments was the following CRISPR-off fusion protein:

(SEQ ID NO: 663)
MGTMPKKKRKVPKKKRKVYNHDQEFDPPKVYPPVPAEKRKPIRVLSLEDGI
ATGLLVLKDLGIQVDRYIASEVCEDSITVGMVRHQGKIMYVGDVRSVTQK
HIQEWGPFDLVIGGSPCNDLSIVNPARKGLYEGTGRLFFEFYRLLHDARP
KEGDDRPFFWLFENVVAMGVSDKRDISRFLESNPVMIDAKEVSAAHRARY
FWGNLPGMNRPLASTVNDKLELQECLEHGRIAKFSKVRTITTRSNSIKQG
KDQHFPVEMNEKEDILWCTEMERVFGFPVHYTDVSNMSRLARQRLLGRSW
SVPVIRHLFAPLKEYFACVSSGNSNANSRGPSFSSGLVPLSLRGSHMAAI
PALDPEAEPSMDVILVGSSELSSSVSPGTGRDLIAYEVKANQRNIEDICI
CCGSLQVHTQHPLFEGGICAPCKDKFLDALFLYDDDGYQSYCSICCSGET
LLICGNPDCTRCYCFECVDSLVGPGTSGKVHAMSNWVCYLCLPSSRSGLL
QRRRKWRSQLKAFYDRESENPLEMFETVPVWRRQPVRVLSLFEDIKKELT
SLGFLESGSDPGQLKHVVDVTDTVRKDVEEWGPFDLVYGATPPLGHTCDR
PPSWYLFQFHRLLQYARPKPGSPRPFFWMFVDNLVLNKEDLDVASRFLEM
EPVTIPDVHGGSLQNAVRVWSNIPAIRSRHWALVSEEELSLLAQNKQSSK
LAAKWPTKLVKNCFLPLREYFKYFSTELTSSLGGPSSGAPPPSGGSPAGS
PTSTEEGTSESATPESGPGTSTEPSEGSAPGSPAGSPTSTEEGTSTEPSE
GSAPGTSTEPSELEDKKYSIGLAIGTNSVGWAVITDEYKVPSKKFKVLGN
TDRHSIKKNLIGALLFDSGETAEATRLKRTARRRYTRRKNRICYLQEIFS
NEMAKVDDSFFHRLEESFLVEEDKKHERHPIFGNIVDEVAYHEKYPTIYH
LRKKLVDSTDKADLRLIYLALAHMIKFRGHFLIEGDLNPDNSDVDKLFIQ
LVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTE
ELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKP
AFLSGEQKKAIVDLLFKTNRKVTVKQLKEDYFKKIECFDSVEISGVEDRF
NASLGTYHDLLKIIKDKDELDNEENEDILEDIVLILILFEDREMIEERLK
TYAHLEDDKVMKQLKRRRYTGWGRLSRKLINGIRDKQSGKTILDFLKSDG
FANRNFMQLIHDDSLTFKEDIQKAQVSGQGDSLHEHIANLAGSPAIKKGI
LQTVKVVDELVKVMGRHKPENIVIEMARENQTTQKGQKNSRERMKRIEEG
IKELGSQILKEHPVENTQLQNEKLYLYYLQNGRDMYVDQELDINRLSDYD
VDAIVPQSFLKDDSIDNKVLTRSDKNRGKSDNVPSEEVVKKMKNYWRQLL
NAKLITQRKEDNLTKAERGGLSELDKAGFIKRQLVETRQITKHVAQILDS
RMNTKYDENDKLIREVKVITLKSKLVSDERKDFQFYKVREINNYHHAHDA
YLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSP
TVAYSVLVVAKVEKGKSKKLKSVKELLGITIMERSSFEKNPIDFLEAKGY
KEVKKDLIIKLPKYSLFELENGRKRMLASAGELQKGNELALPSKYVNFLY
LASHYEKLKGSPEDNEQKQLFVEQHKHYLDEIIEQISEFSKRVILADANL
DKVLSAYNKHRDKPIREQAENIIHLFTLTNLGAPAAFKYFDTTIDRKRYT
STKEVLDATLIHQSITGLYETRIDLSQLGGDSPKKKRKVGVDGSSGSETP
GTSESATPESTGMNNSQGRVTFEDVTVNFTQGEWQRLNPEQRNLYRDVML
ENYSNLVSVGQGETTKPDVILRLEQGKEPWLEEEEVLGSGRAEKNGDIGG
QIWKPKDVKESLSADYKDDDDKAPKKKRKVPKKKRKV

Human primary T cells obtained from human donors using standard protocols were thawed and cultured under standard T-cell culture conditions. An exemplary outline of the general experimental design for certain multiplexing experiments described herein is provided in FIG. 1. Briefly, effector proteins, such as epigenetic repressor fusion proteins (e.g., CRISPR-off fusion proteins), wildtype Cas9 protein, or dCas9 protein, was delivered to the primary T cells in the form of mRNA via nucleofection. Where a guide RNA or a combination of guide RNAs was delivered, mRNA and guide RNA(s) were nucleofected together. Nucleofection was performed at different time points after thawing the primary human T cells, e.g., on day 2 or day 3 after thawing, and the day of nucleofection was designated day 0 of the experiment. Assessments of the effects of the various repressive proteins was performed at specific times after nucleofection, e.g., an assessment at day 7 was performed on the seventh day after nucleofection, a day 13 assessment on day 14 was performed on the 14^{th} day after nucleofection, and so on. Where cells were modified to express a chimeric antigen receptor (CAR), the CAR was delivered to the cells via transduction with a lentiviral vector encoding the CAR. Lentiviral transduction was performed at different times after thawing the cells, as indicated in the context of the respective experiments.

B2M expression was measured by cell surface B2M staining and flow cytometry. CIITA and RFX5 expression was assessed by measuring cell surface expression of HLA-DR by flow cytometry (as it is believed that CIITA and RFX5 are required for HLA-DR expression). TRAC, CD247, CD3D, and CD3G expression was assessed by measuring surface expression of CD3/TCR by flow cytometry (as each one of these genes is believed to be required for CD3/TCR expression on the surface of T cells).

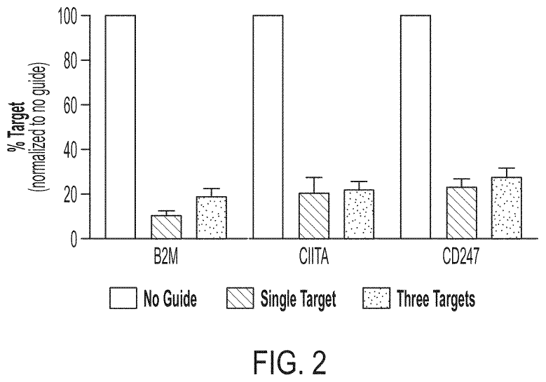

An initial multiplexing experiment was conducted in which multiplex epigenetic silencing of three exemplary genes (B2M, CIITA, and CD247) in primary human T cells was effected. FIG. 2 compares epigenetic silencing activity in T cell populations in which single-gene silencing was performed with epigenetic silencing activity in T cell populations in which multiplex epigenetic gene silencing of all three genes was performed. Guide RNAs used in the multiplexing group included: RNA949 (B2M), RNA138 (B2M), RNA232 (CIITA), RNA268 (CIITA), RNA1409 (CD247), and RNA1566 (CD247). Multiplex silencing showed efficacy close to single-target epigenetic silencing at day 13 (FIG. 2), indicating that multiplex silencing results in treated cells that are characterized by significantly reduced levels of B2M, HLA-DR, and CD3 surface expression.

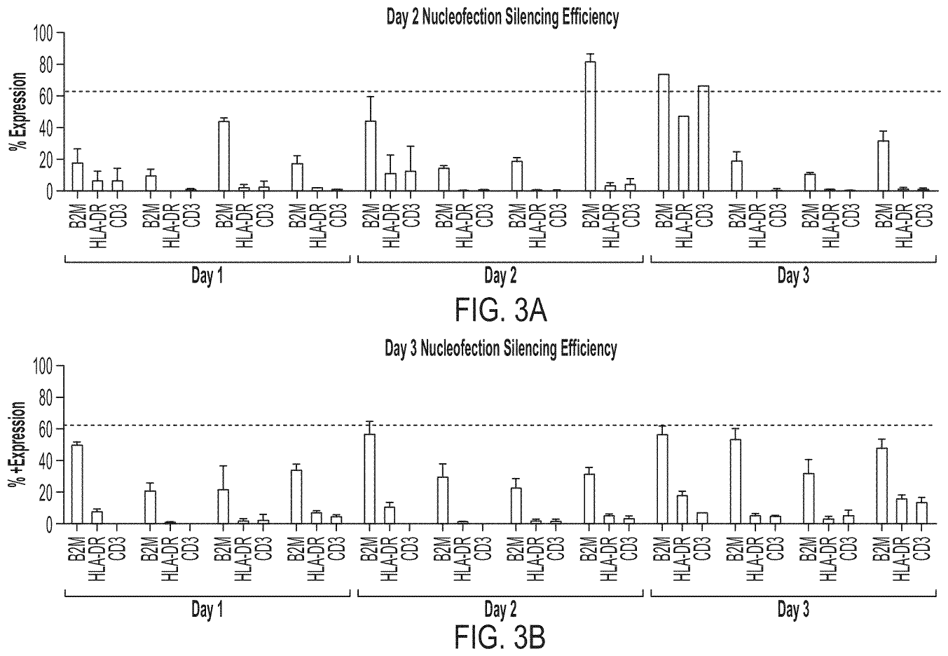

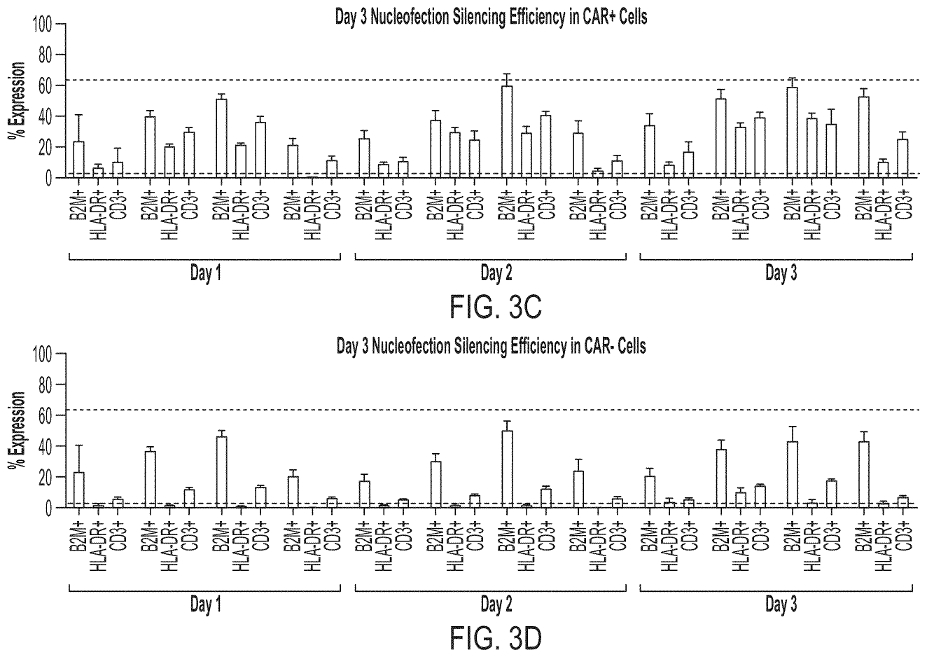

The results of another multiplexing experiment are depicted in FIGS. 3A-3D and show the effect of simultaneous epigenetic silencing of B2M, CIITA, and CD247 in human T cells, using the following six gRNAs: RNA949 (B2M), RNA138 (B2M), RNA232 (CIITA), RNA268 (CIITA), RNA1409 (CD247), and RNA1566 (CD247). CIITA silencing was assessed by measuring HLA-DR cell surface expression and CD247 silencing was assessed by measuring CD3 cell surface expression in the treated cells. FIG. 3A shows epigenetic silencing of target genes after nucleofection of the T cells with epigenetic suppressor (CRISPR-off) and the gRNAs on Day 2, with lentiviral transduction (encoding a BCMA CAR) on Day 1, Day 2, or Day 3. FIG. 3B shows epigenetic silencing of target genes after nucleofection on Day 3, with lentiviral transduction on Day 1, Day 2, or Day 3. FIGS. 3C-3D show epigenetic silencing of target genes after nucleofection on Day 3, with transduction on Day 1, Day 2, or Day 3, in both chimeric antigen receptor (BCMA CAR) positive (FIG. 3C) and CAR negative (empty lentiviral vector, FIG. 3D) cells from the same well.

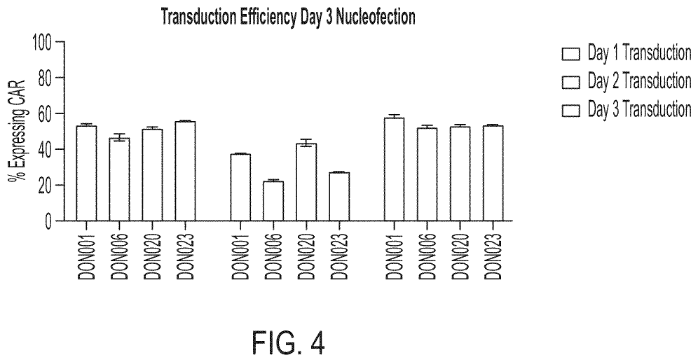

The lentiviral transduction efficiency for day 3 nucleofection was assessed by measuring the percentage of cells expressing the BCMA CAR, and the resulting data are shown, separated by cell donor and day of transfection, in FIG. 4.

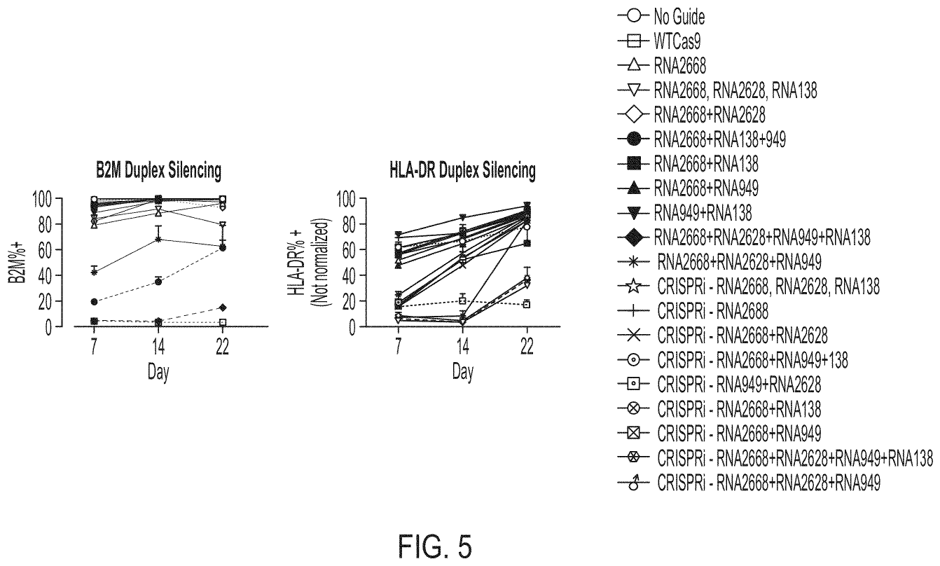

Time course experiments of multiplexed (here: duplexed) epigenetic silencing of B2M (left) and RFX5 (right) in human T cells over 22 days were performed (FIG. 5, guide RNAs as indicated in figure legend). Treated primary human T cells were restimulated at Day 14 with CD3/CD28 antibodies using standard protocols. RFX5 silencing was assessed by measuring cell surface expression of HLA-DR in the treated cell populations.

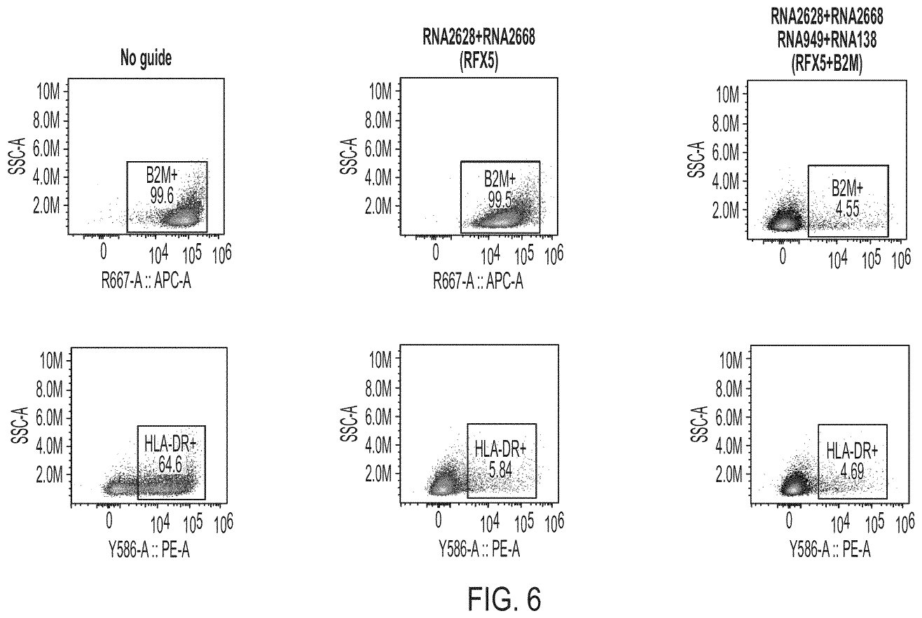

A substantial portion of the duplex-edited (B2M- and RFX5-silenced) primary T cells were determined to be B2M and HLA-DR negative (FIG. 6) as measured by flow cytometry.

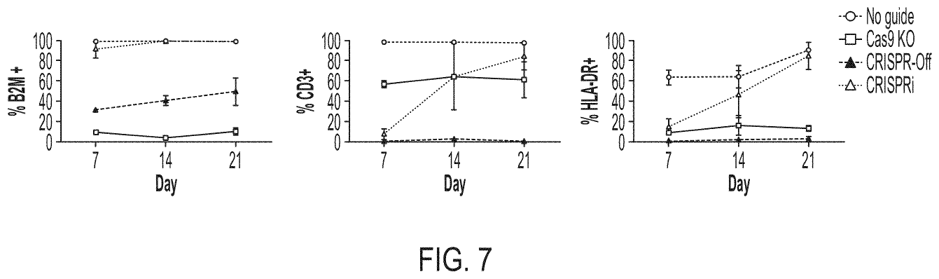

Multiplex silencing of B2M, CD3D/G, and RFX5 was performed in primary human T cells by current methods (Cas9 KO-wtCas9, and CRISPRi-dCas9) and compared to multiplexed epigenetic silencing using an epigenetic repressor fusion protein (CRISPR-Off) as described above together with a combination of gRNAs targeting the three genes (RNA138, RNA949, RNA2628, RNA2668, RNA2329, RNA2510). RFX5 silencing was assessed by measuring cell surface expression of HLA-DR in the treated cell populations. Guides used included RNA138, RNA949, RNA2628, RNA2668, RNA2329, and RNA2510. Controls included wt Cas9 (positive control, Cas9 KO), epigenetic editor without guide (no guide, negative control), CRISPRi (dCas9, negative control for durability). Data shown in FIG. 7 illustrate that multiplexed, epigenetic editing achieved a significantly improved, durable reduction in the portion of CD3 and HLA-DR-expressing cells as compared to both wtCas9 and CRISPRi. B2M expression was also significantly reduced in cell populations treated with the multiplexed epigenetic editor, albeit not to the level observed in wtCas9 treated cells.

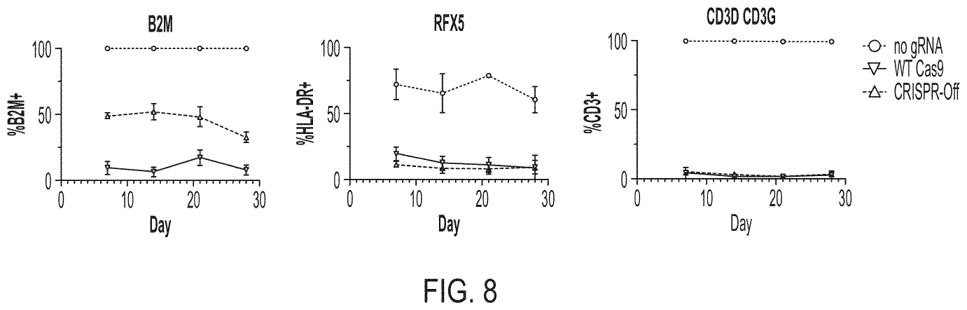

Similar results were observed in experiments performing multiplex silencing of B2M, RFX5, and CD3D/G in primary human T cells expressing a BCMA CAR over time (FIG. 8). Guides used included RNA138, RNA949, RNA2628, RNA2668, RNA2329, and RNA2510. At Day 14 cells were restimulated with BCMA and RPMI 8226 cells to test durability of silencing post-CAR stimulation. Data shown in FIG. 8 illustrate that multiplexed, epigenetic editing achieved a durable reduction in the portion of CD3 and HLA-DR-expressing cells that was comparable in its level to the level of repression observed in the wtCas9 treated cells and significantly improved over the CRISPRi treated cells. B2M expression was also significantly reduced in cell populations treated with the multiplexed epigenetic editor, albeit not to the level observed in wtCas9 treated cells.

Allogeneic response was tested using an allo-stealth MLR assay. WT T cells (effector) were derived from a donor and silenced or knocked out for B2M, RFX5, or both genes. PMBCs were derived from another donor and used as target cells. The effector and target cells were combined in a 1:1 ratio and alloresponse was tested using a CellTrace Violet (CTV) assay, and % diluted CTV was used as a measure of proliferation. In both CD8 and CD4 T cells, proliferation was more greatly reduced by multiplexing epigenetic silencing of both B2M and RFX5 than by silencing either gene alone.

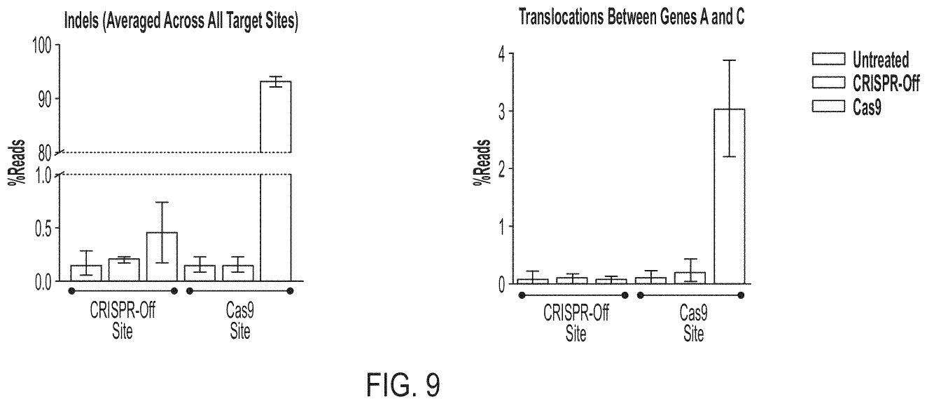

Next, the frequency of adverse mutation events was compared between multiplexed gene editing and multiplexed epigenetic editing methodologies. For these experiments, primary human T cll populations were either treated with wildtype Cas9 with guide RNAs targeting B2M (RNA104), CIITA (RNA464), and CD247 (RNA1307), or with an epigenetic repressor fusion protein (CRISPR-off) targeting the same genes. The guide RNAs used for the epigenetic multiplexed silencing included: RNA110 (B2M), RNA112 (B2M), RNA258 (CIITA), RNA269 (CIITA), RNA1473 (CD247), and RNA1544 (CD247). The results are shown in FIGS. 9-26.

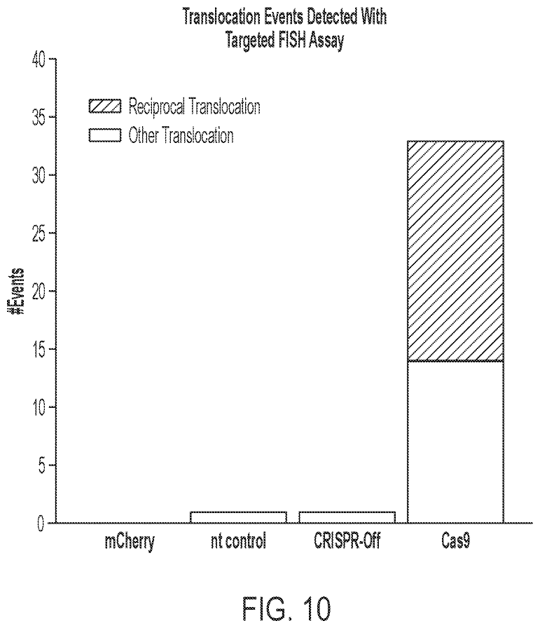

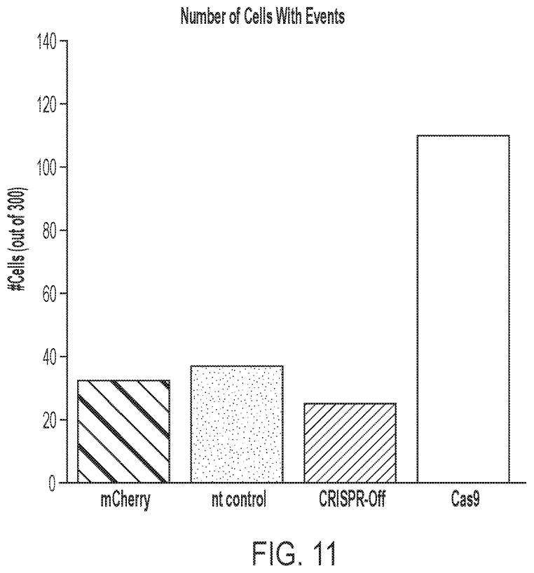

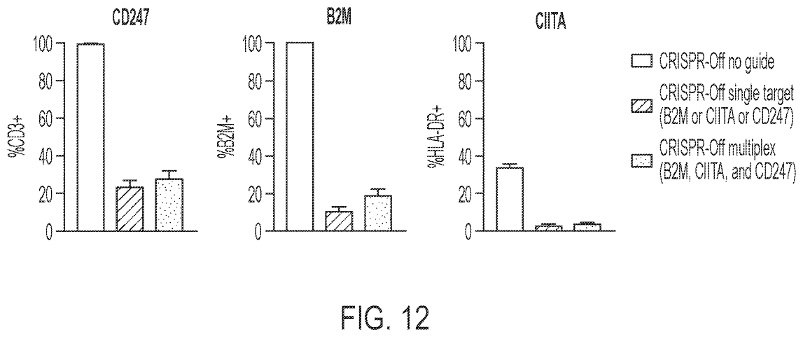

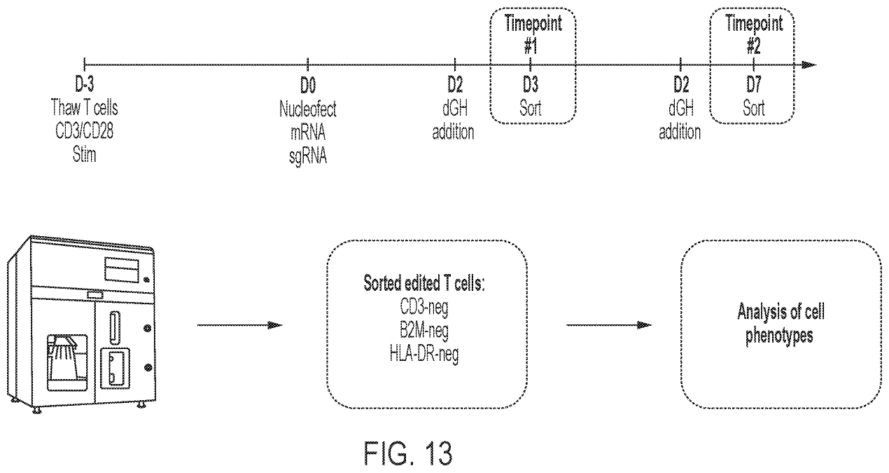

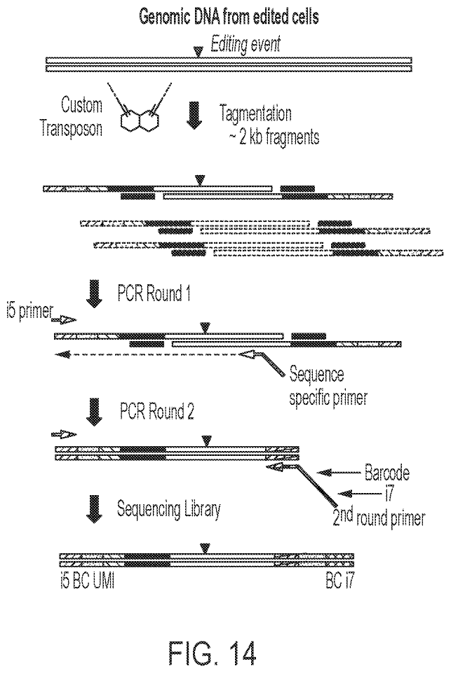

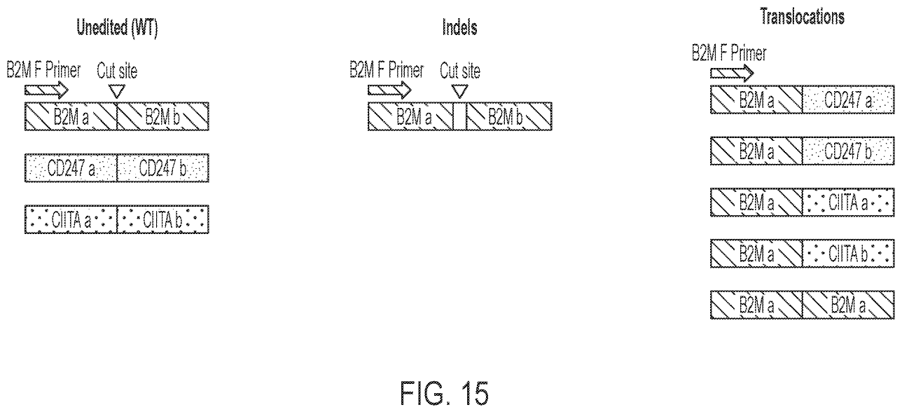

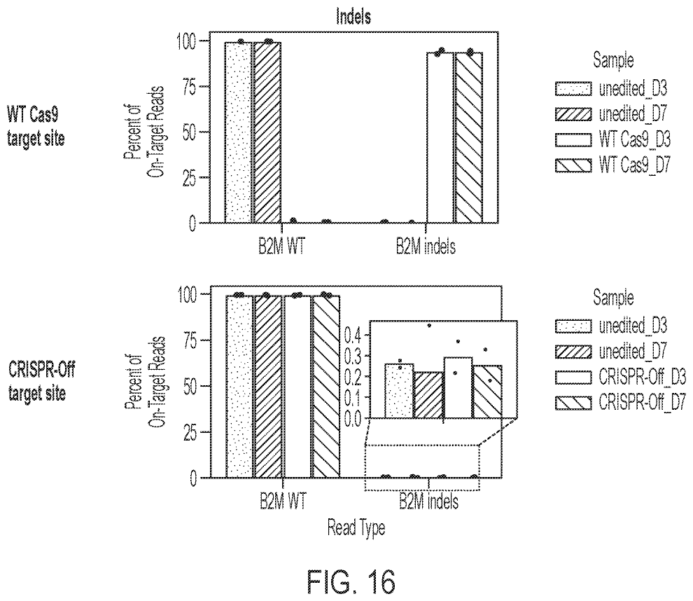

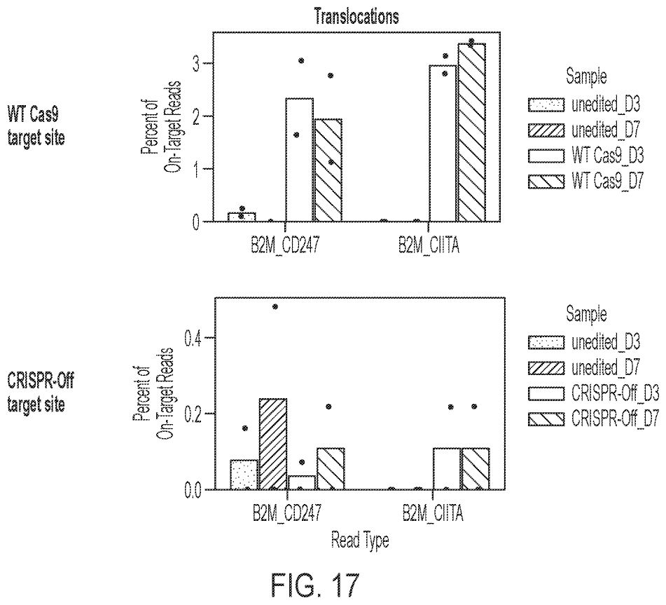

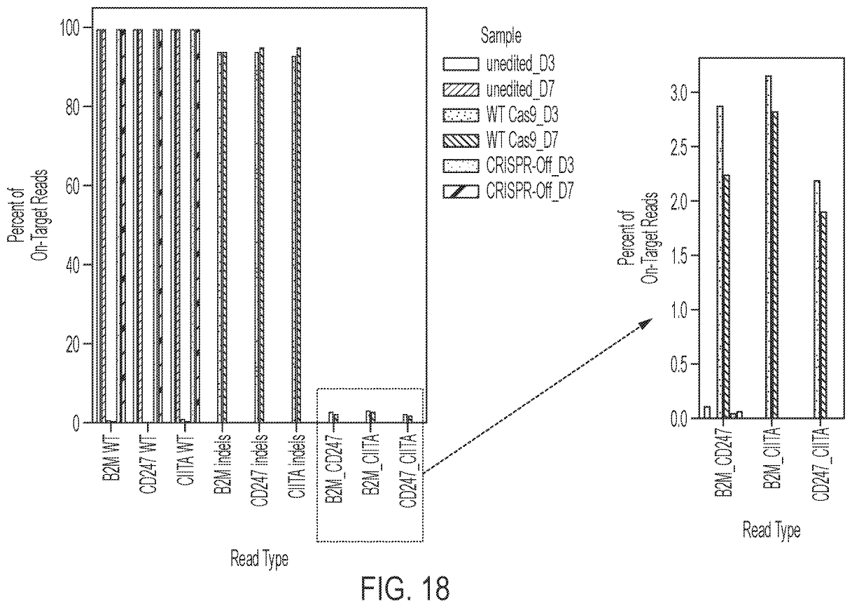

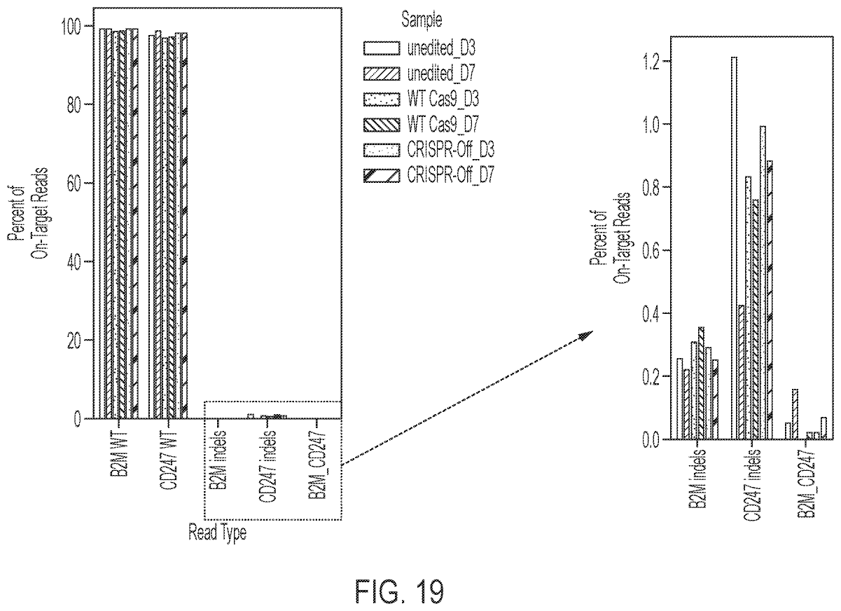

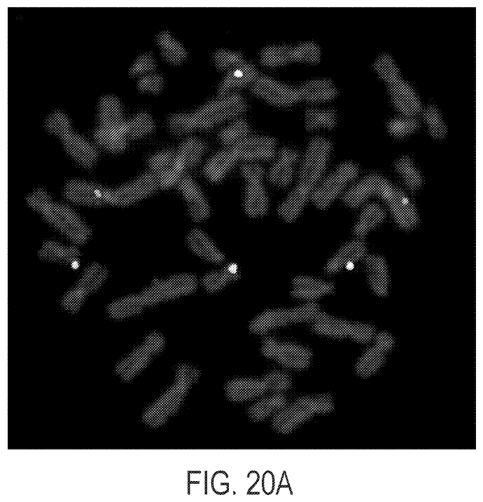

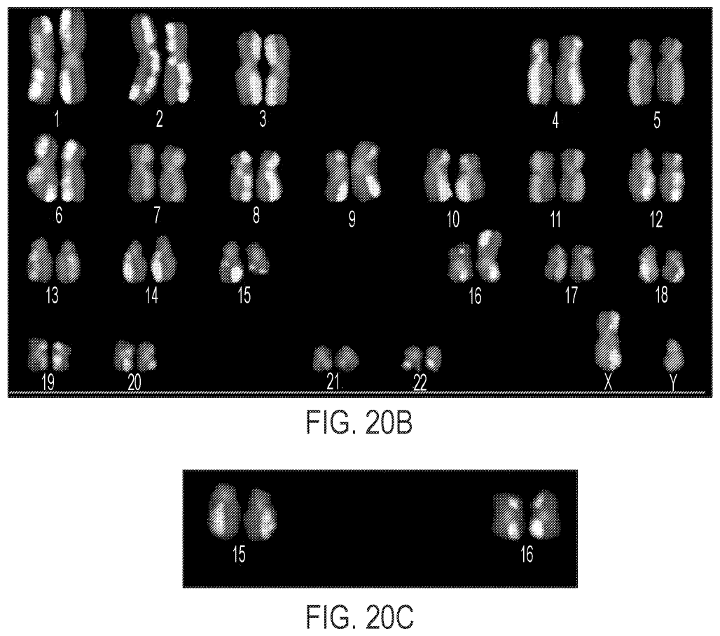

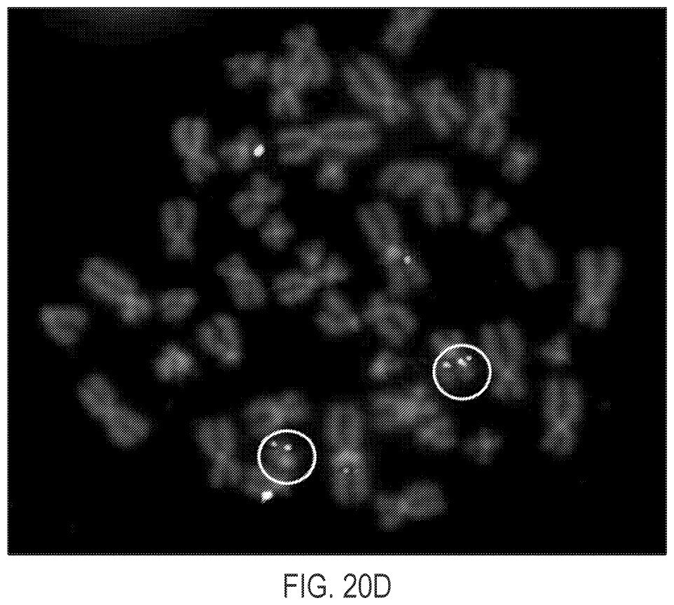

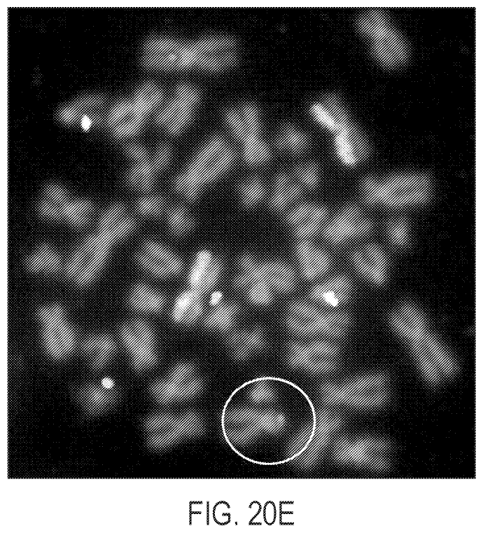

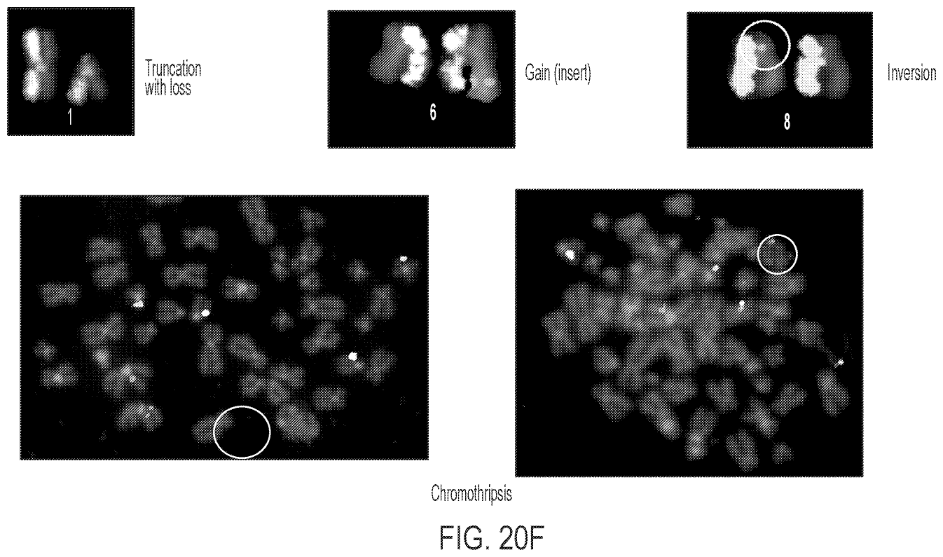

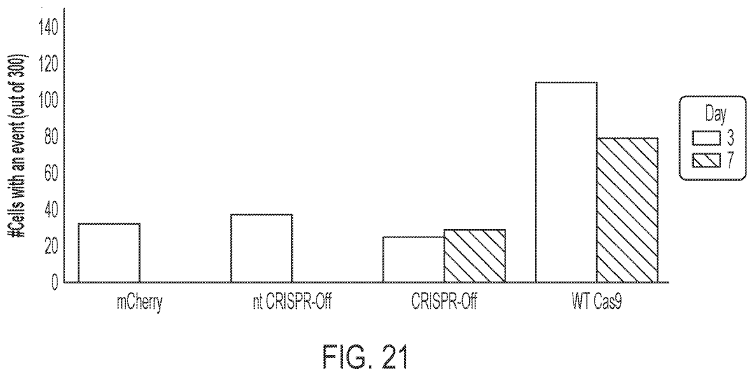

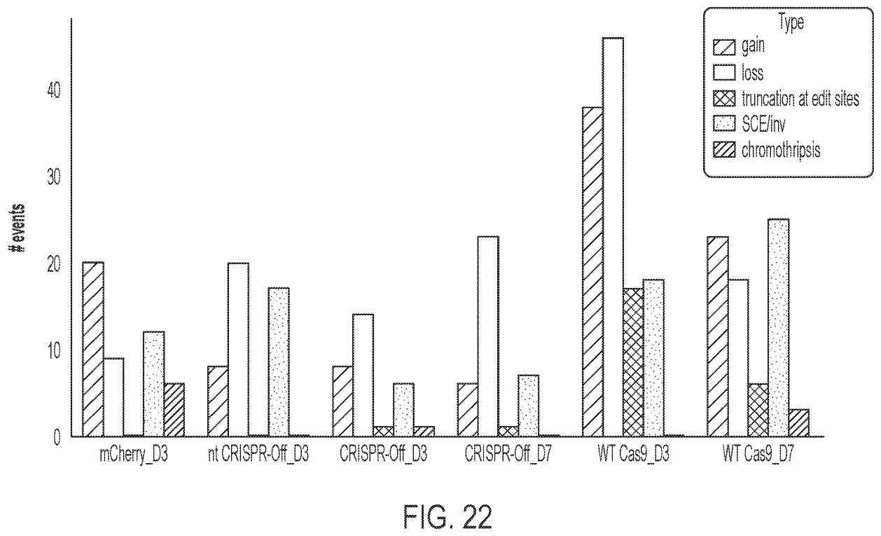

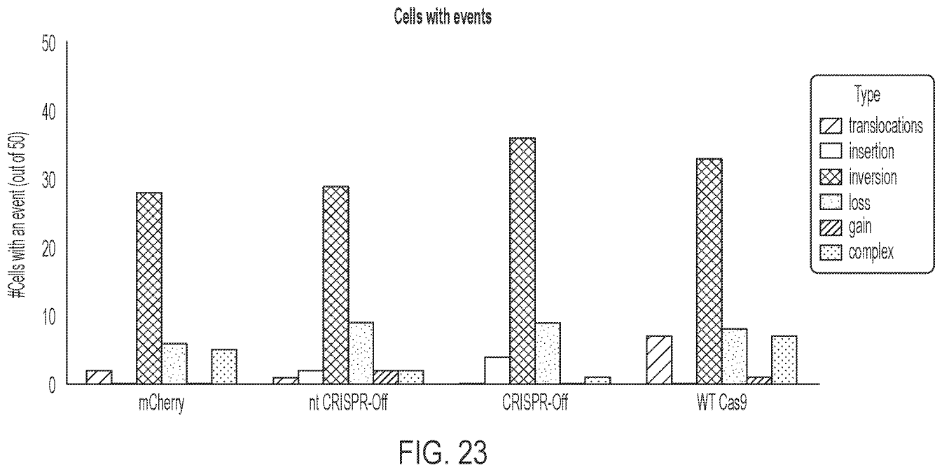

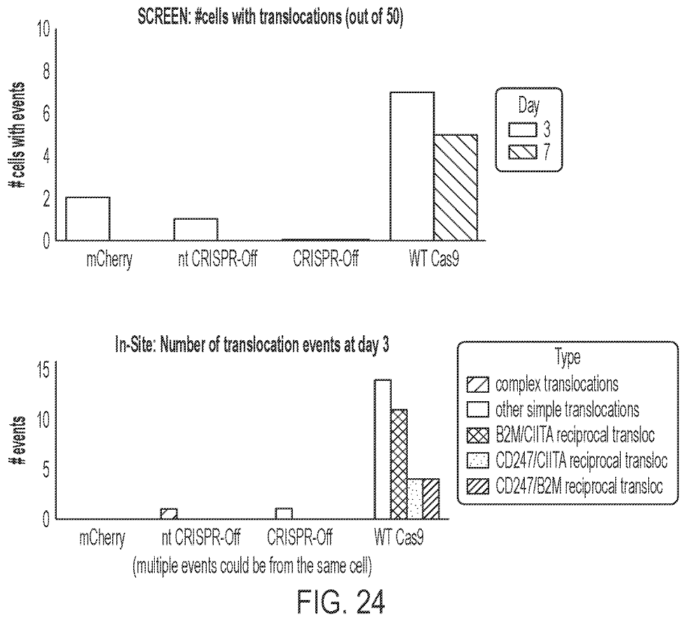

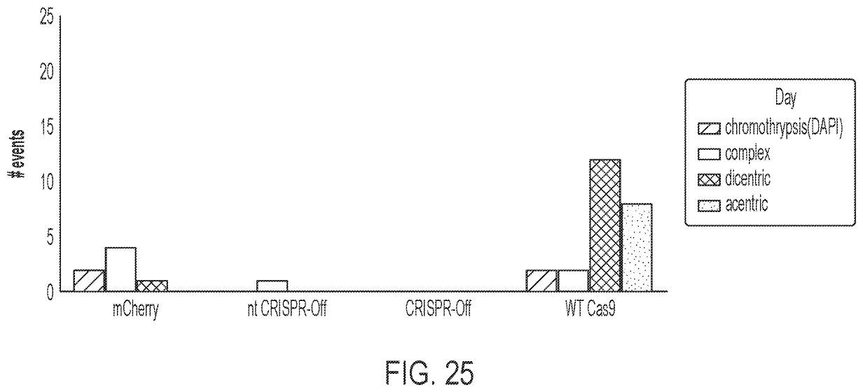

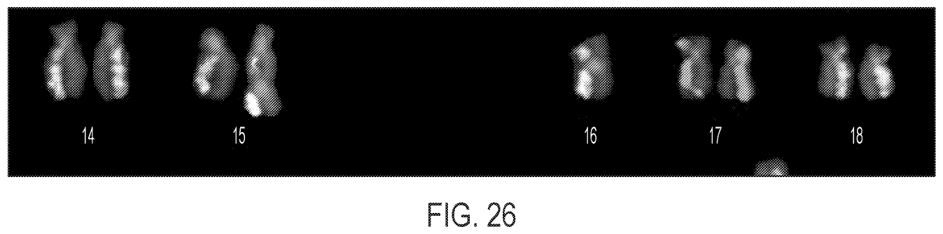

FIG. 9 shows that cells treated with exemplary epigenetic repressor fusion proteins (CRISPR-off) of this disclosure and multiplex targeting guide RNA combinations show significantly fewer adverse genetic events, such as indels (left) or translocations (right) compared to current Cas9-based, DNA-cutting gene editing methods. Events were identified using an unbiased, sequence-based methodology. FIG. 10 shows a graph of translocation events in treated cells observed using fluorescence in situ hybridization (FISH). FIG. 11 shows a graph of genomic rearrangement events in treated cells observed using fluorescence in situ hybridization (FISH). FIG. 12 shows silencing of CD247, B2M and CIITA in multiplex epigenetically edited cells used to assess genomic integrity post-epigenetic editing. FIG. 13 shows a non-limiting experimental workflow for assessing genomic integrity in epigenetically edited cells. FIG. 14 shows a diagram of a non-limiting example of an unbiased, sequence-based methodology. FIG. 15 is a diagram showing examples of genetic events that may be detected by unbiased, sequence-based methods, such as indels (middle) and translocations (right). FIG. 16 shows the percent of B2M reads with indel events in cells treated with current CRISPR methods (top) or a non-limiting example of the compositions of this disclosure (bottom). FIG. 17 shows the percent of B2M reads with translocation events in cells treated with current CRISPR methods (top) or a non-limiting example of the compositions of this disclosure (bottom). FIG. 18 shows all indel and translocation events observed in wildtype Cas9-target sites after treatment of primary human T cells with either multiplexed wtCas9 gene editing systems targeting B2M, CD247, and CIITA; multiplexed epigenetic editor fusion proteins (CRISPR-off) targeting B2M, CD247, and CIITA; and in unedited human primary T cells. For each genotype observation, there are three pairs of bars. The first two bars show the occurrence of the genotype in an unedited cell; the second pair indicate the occurrence of the genotype in wild type Cas9-edited cells; and the third pair indicate the occurrence of the genotype in epigenetically edited cells. Within each pair, the left bar indicates day 3 results and the right bar indicates day 7 results. WT: naturally-occurring (wildtype) genomic sequence at the target site; indels: indel mutation at the target site; translocations are expressed by a pairing of genes, e.g., B2M_CD247 indicate a translocation between the B2M and CD247 target sites. FIG. 19 shows all indel and translocation events observed in target sites used for multiplex epigenetic editing of B2M and CD247 after treatment of primary human T cells with either multiplexed wtCas9 gene editing systems targeting B2M, CD247, and CIITA; multiplexed epigenetic editor fusion proteins (CRISPR-off) targeting B2M, CD247, and CIITA; and in unedited human primary T cells. Only data for B2M and CD247 target sites are shown. For each genotype observation, there are three pairs of bars. The first two bars show the occurrence of the genotype in an unedited cell; the second pair indicate the occurrence of the genotype in wild type Cas9-edited cells; and the third pair indicate the occurrence of the genotype in epigenetically edited cells. Within each pair, the left bar indicates day 3 results and the right bar indicates day 7 results. FIGS. 20A-F show directional genomic hybridization images indicating genetic events in treated cells. FIG. 20A shows cells with no adverse events. FIG. 20B shows a reciprocal translocation between B2M (chr. 15) and CIITA (chr. 16) on day 7 in wildtype Cas9 treated cells. FIG. 20C shows chromosomes 15 and 16 in a cell with no translocations for comparison. FIG. 20D shows a reciprocal translocation between B2M and CIITA on day 3 in wildtype Cas9-treated cells. FIG. 20E shows an edit site truncation at CD247 on day 7 in wildtype Cas9-treated cells. FIG. 20F shows cells with additional abnormalities. FIG. 21 shows a summary of genomic event occurrences observed. FIG. 22 shows a summary of genomic events observed by type of event as measured by in-Site. Within each group of bars, the types of events from left to right are: gain, loss, truncation at edit sites, SCE/inv, and chromothripsis. FIG. 23 shows a summary of genomic events observed by type of event as measured by SCREEN. Within each group of bars, the types of events from left to right are: translocations, insertion, inversion, loss, gain, and complex. FIG. 24 shows a summary of translocation occurrences using SCREEN (top) and In-Site (bottom). Within each group of bars for the In-Site panel, the types of events from left to right are: complex translocations, other simple translocations, B2M/CIITA reciprocal translocations, CD247/CIITA reciprocal translocations, and CD247/B2M reciprocal translocations. FIG. 25 shows a summary of dicentric, acentric, and complex events. Within each group of bars, the types of events from left to right are: chromothripsis (DAPI), complex, dicentric, and acentric. FIG. 26 shows a translocation observed, at day 3 in a triple Cas9 KO sample, between Chr15q and Chr16p forming a dicentric event. The acentric partner is missing from the spread leading to monosomy of Chr 16.

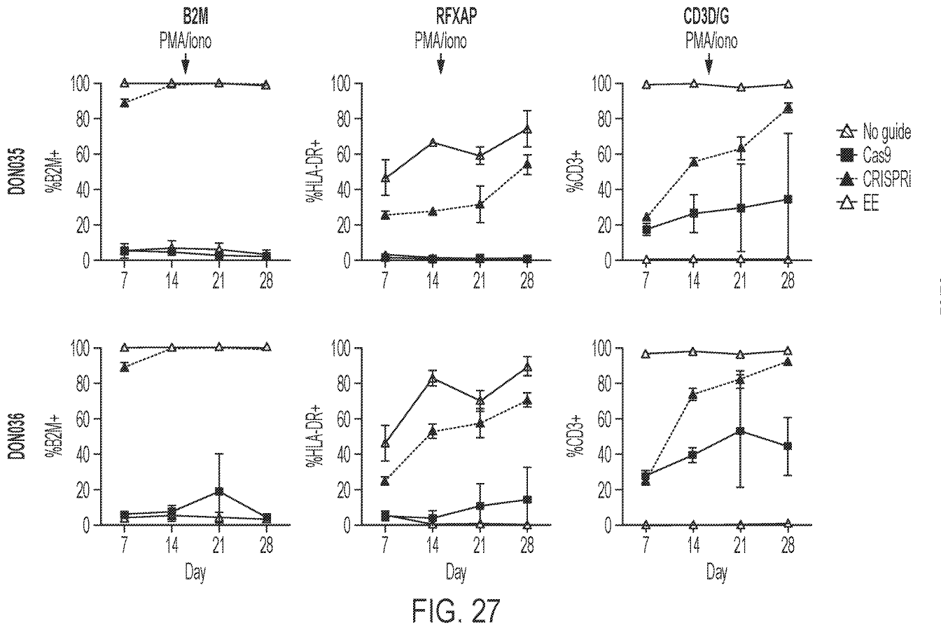

Multiplex silencing of B2M, CD3D/G, and RFXAP was performed in primary human T cells from two donors by current methods (Cas9 KO-wtCas9, and CRISPRi-dCas9) and compared to multiplexed epigenetic silencing using an epigenetic repressor fusion protein (CRISPR-Off) as described above together with a combination of gRNAs targeting the three genes (RNA138, RNA988, RNA2977, RNA2329, RNA2510). 2.5 ug mRNA, 3 ug each B2M guide, 2.5 ug RFXAP guide, and 0.5 ug each CD3 guide were used in each nucleofection well. RFXAP silencing was assessed by measuring cell surface expression of HLA-DR in the treated cell populations. Controls included wt Cas9 (positive control, Cas9 KO), epigenetic editor without guide (no guide, negative control), CRISPRi (dCas9, negative control for durability). Data shown in FIG. 27 illustrate that multiplexed, epigenetic editing achieved a comparable or significantly improved, durable reduction in B2M, CD3, and HLA-DR as compared to both wtCas9 and CRISPRi through a PMA/ionomycin restimulation at Day 14.

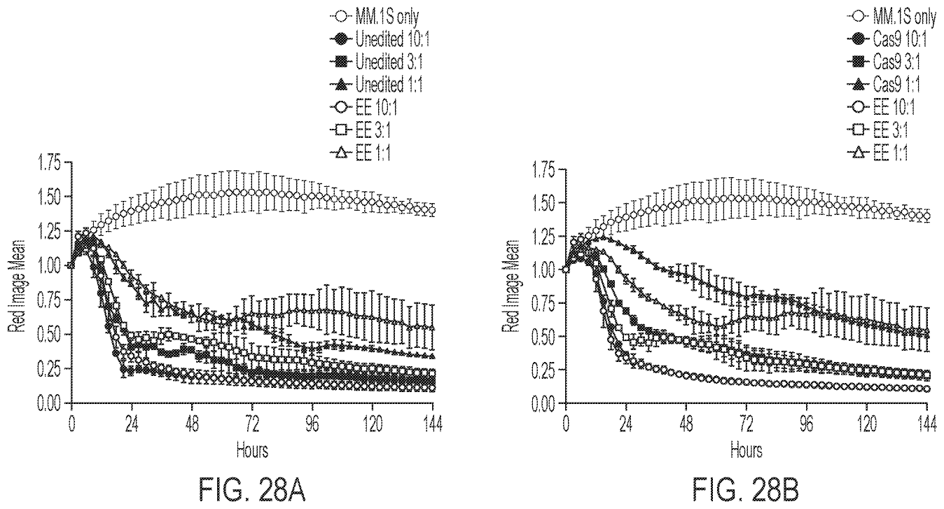

CAR-based repeat stimulation was performed by restimulating with either PMA/iono or tumor restimulation at Day 14, followed by tumor restimulation at Day 17, Day 21, and D24. % B2M+, % RFXAP+, and % CD3+ was tested at four time points, with silencing of each gene being durable through multiple tumor restimulations. Potency of CAR T cells epigenetically silenced for B2M, CD3D/G, and RFXAP was tested in an Incucyte killing assay using MM.1S myeloma cells. Multiplex silencing of B2M, CD3D/G, and RFXAP was performed in primary human T cells by multiplexed epigenetic silencing using an epigenetic repressor fusion protein (CRISPR-Off) as described above together with a combination of gRNAs targeting the three genes (RNA138, RNA988, RNA2977, RNA2329, RNA2519). 2.5 ug mRNA, 3 ug each B2M guide, 2.5 ug RFXAP guide, and 0.5 ug each CD3 guide were used in each nucleofection well. For the killing assay, several different effector:target ratios were used, as well as a myeloma cell-only control sample. Red Image Mean was used as a measure of cell survival, as the MM.1S cells were transduced to express a red fluorescent marker. The fluorescence intensity for each timepoint was normalized to the initial imaging timepoint, given as time 0 (x-axis). Images were taken every three hours for a total of six days to track CAR-mediated killing over time. A reduction in the normalized red image mean (y-axis)—as indicated by values below 1—shows loss of tumor cells due to CAR T cell-mediated killing. Triple silenced CAR T have similar initial killing potency to unedited CAR T (FIG. 28A) and potentially improved potency compared to triple KO CAR T (FIG. 28B).

### Example 4: Design and Selection of HLA-E Peptide Variants and NK Degranulation Assay

A total of 5 peptide variants, derived from HLA class I leader sequences and loaded to an HLA-E fusion protein, were designed based on previous work showing protection from NKG2C+ cytotoxicity (Llano et al., Eur. J. Immunol. 1998). These 5 peptide variants include VMAPRTLIL (SEQ ID NO: 674), VMAPRTLLL (SEQ ID NO: 675), VMAPRTLVL (SEQ ID NO: 676), VMAPRTVLL (SEQ ID NO: 677), and VMAPRTLFL (SEQ ID NO: 678) and were constructed in an EF1A-HLAE-WPRE lentiviral vector.

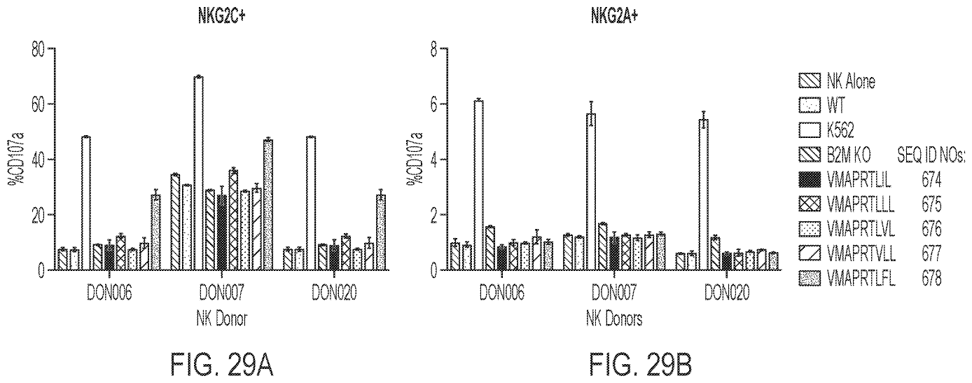

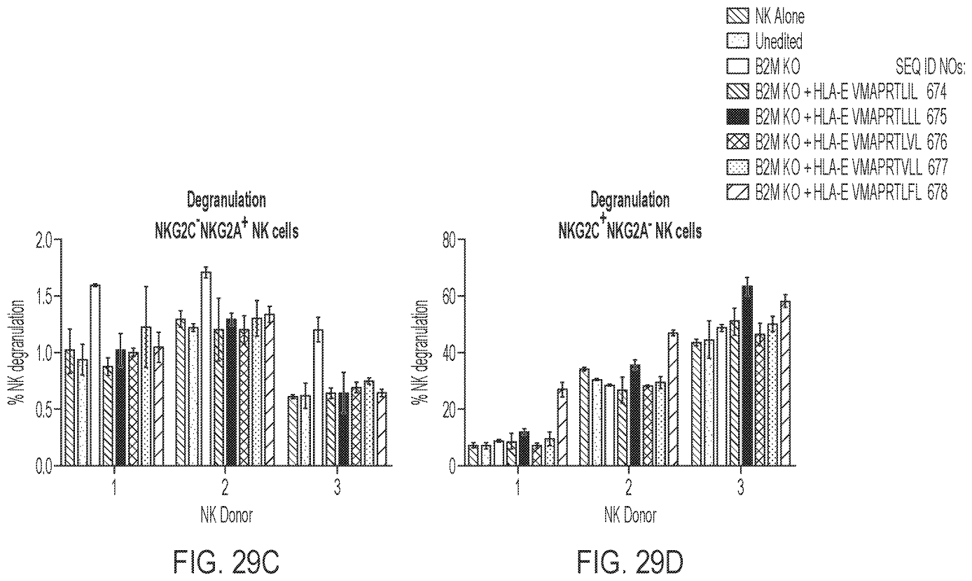

An NK degranulation assay was performed to assess NK cell responses to B2M knockout T cells expressing HLA-E with different peptide variants. T cells were transduced with a lentiviral construct consisting of each of the HLA-E fusion proteins with the peptide variants, and then nucleofected with an mRNA complex consisting of 2.5 ug of CRISPR-Cas9 and 1.25 ug of a B2M-target Guide RNA (gRNA) to generate B2M T cell knockouts. B2M- and B2M-HLAE+ T cells were sorted by FACS and then expanded for 1 week. The evening before the degranulation, NK cells from 3 healthy donors were thawed and cultured in NK MACS medium (Miltenyi Biotec) supplemented with 1000 ng/ml of IL-2 (Miltenyi Biotec), 5 ng/ml of IL-15 (Peprotech), and 5% Human AB serum (GemCell) and incubated overnight at 37 C and 5% CO2. The following morning, T cells and K562s were stained with 5 mM CFSE for 3 min at RT and then co-cultured with NK cells at a 1:1 ratio in a 96-well U-bottom plate in 150 ul of medium with 1 ul of anti-CD107a antibody (Biolegend). After 1 h, 50 ul of pre-warmed media containing Monensin (Biolegend) was added to the co-cultures and incubated for an additional 5h. Cells were then stained with anti-NKG2A (Beckman Coulter), anti-NKG2C (Biolegend), anti-CD3 (Biolegend), Zombie-NIR viability dye (Biolegend), anti-CD56 (Biolegend) antibodies and then assessed by flow cytometry. Results for NKG2C+ and NKG2A+ cells as determined by flow cytometry are shown in FIGS. 29A and 29B, respectively. NK degranulation results are shown in FIGS. 29C and 29D. HLA-E variants that induced the lowest CD107a expression on NKG2C+ NK cells were determined as the least triggering of NK cytotoxicity.

Together, the results shown here demonstrate that multiplexed epigenetic silencing achieves robust, durable repression of multiple target genes with greatly reduced frequencies of detrimental mutation events as compared to conventional gene editing methods.

### Example 5: Specific Killing of Primary T Cells Transduced with HLA-E Variants

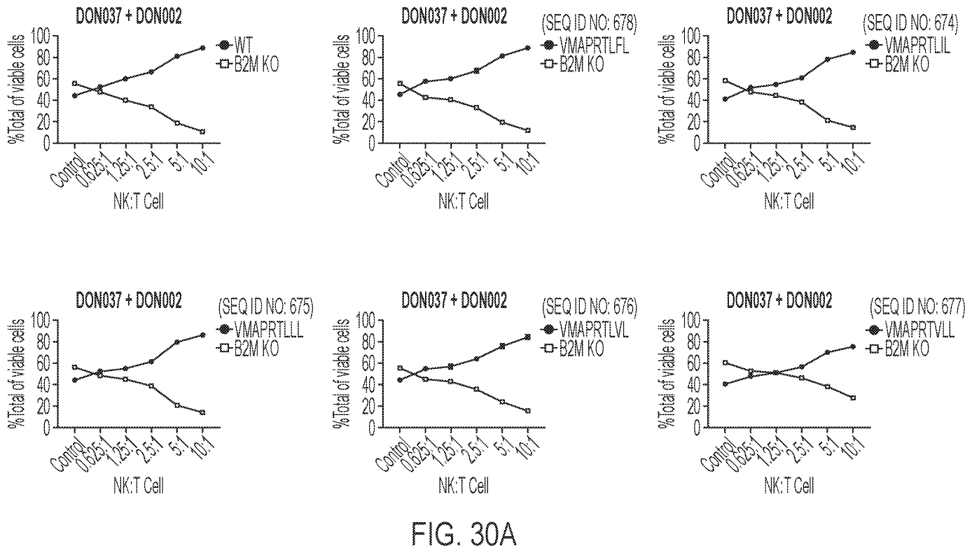

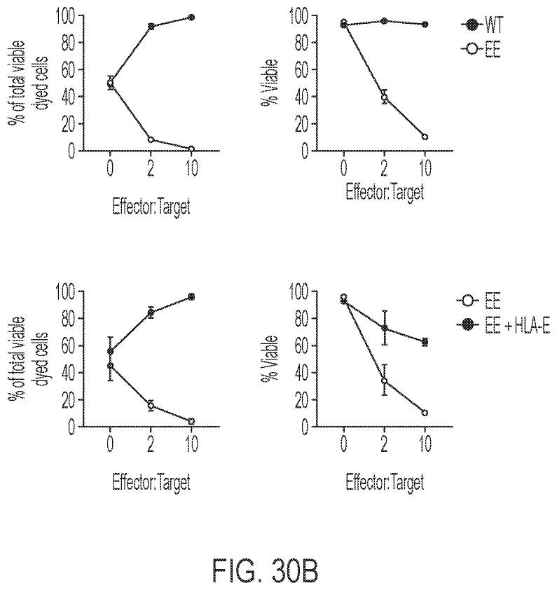

HLA-E variants were assessed for the ability to reduce NK cell lysis of B2M knockout (KO) T cells in a specific killing assay. Sequences for the transduced HLA-E transgenes are SEQ ID NOs: 667-672. Cells were engineered as above to produce sorted B2M KO T cells expressing an HLA-E variant. The evening before the specific killing assay, NK cells from 2 healthy donors that had previously been expanded and activated for 12 days in NK MACS medium (Miltenyi Biotec, Cat. No. 130-114-429) supplemented with 1000 ng/ml of IL-2 (Miltenyi Biotec), 20 ng/ml of IL-15 (Peprotech), and 10% Human AB serum (GemCell, Cat. No. 100-512), were thawed and cultured overnight in similar media. The following morning, B2M KO T cells were stained with 0.5 μM CFSE (BioLegend, Cat. No. 423801) for 3 min at RT and B2M KO+HLA-E T cells or WT T cells were stained with 1 μM CellTrace Violet (CTV; Invitrogen, Cat. No. C34557) for 5 min at RT. The indicated CFSE- and CTV-stained cells were mixed at approximately a 1:1 ratio and the mixture co-cultured with NK cells in a 96-well U-bottom plate in 200 ul of medium at the NK:T cell ratios indicated. Approximately 24 hours later, co-cultures were stained with Zombie-NIR viability dye (BioLegend, Cat. No. 423106) and assessed by flow cytometry. Results for various co-culture conditions are shown in FIG. 30A. Results for a similar experiment using a B2M epigenetic editor rather than a knockout are shown in FIG. 30B. An increase in the frequency of HLA-E+ cells with increasing NK:T cell ratio was determined to indicate protection of B2M KO HLA-E variant-expressing cells from NK cell lysis relative to the susceptibility of B2M KO T cells to NK killing.

### Example 6: Specific Killing of K562 Cells Transduced with HLA-E Variants

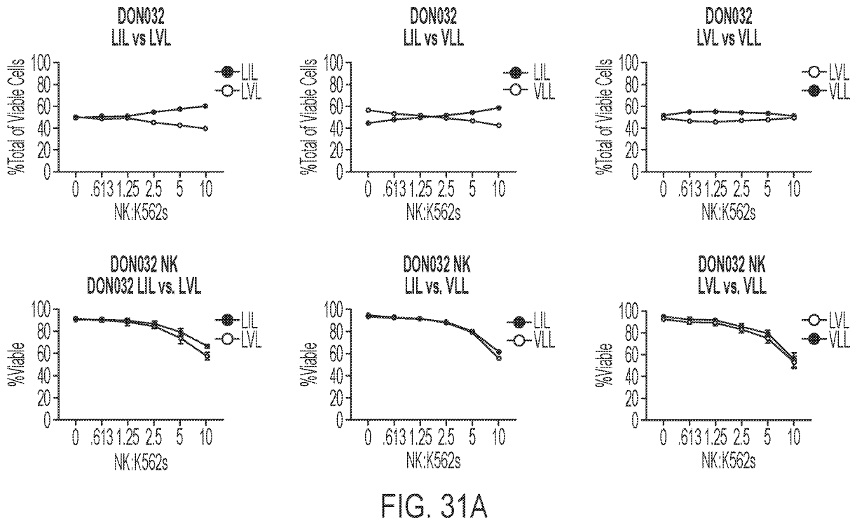

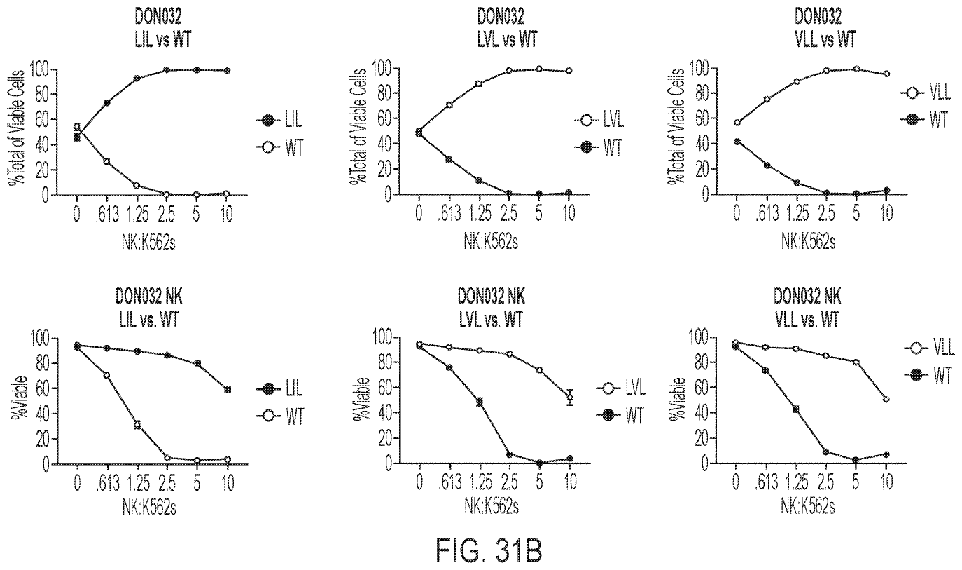

HLA-E variants were assessed for the ability to reduce NK cell lysis of K562 cells, which are highly susceptible to NK cell killing, in a specific killing assay. Sequences for the transduced HLA-E transgenes are SEQ ID NOs. 667-672. The evening before the specific killing assay, NK cells from 2 healthy donors that had previously been expanded and activated for 12 days in NK MACS medium (Miltenyi Biotec, Cat. No. 130-114-429) supplemented with 1000 ng/ml of IL-2 (Miltenyi Biotec), 20 ng/ml of IL-15 (Peprotech), and 10% Human AB serum (GemCell, Cat. No. 100-512), were thawed and cultured overnight in similar media. The following morning, K562 cells with or without expression of HLA-E variants were stained with 0.5 M CFSE (BioLegend, Cat. No. 423801) for 3 min at RT or 1 μM CellTrace Violet (CTV; Invitrogen, Cat. No. C34557) for 5 min at RT. In each assay, CFSE- and CTV-stained cells were mixed at approximately a 1:1 ratio and the mixture co-cultured with NK cells in a 96-well U-bottom plate in 200 ul of medium at the NK:K562 ratios indicated. Approximately 24 hours later, co-cultures were stained with Zombie-NIR viability dye (BioLegend, Cat. No. 423106) and assessed by flow cytometry. Results for the indicated co-culture conditions are shown in FIGS. 31A and 31B. An increase in the frequency of HLA-E variant positive cells with increasing NK:K562 ratio and a maintenance of higher viability within the HLA-E variant-positive population was determined to indicate protection of K562 HLA-E variant-expressing cells from NK cell lysis relative to the susceptibility of the other K562 cell population to NK killing.

### Example 7: Silencing of Three Allo Targets in Primary T Cells

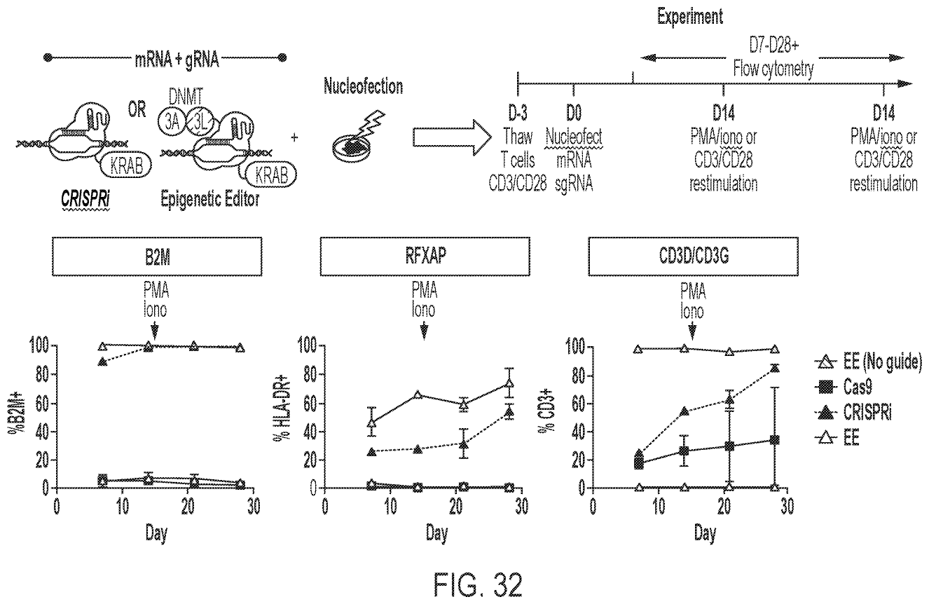

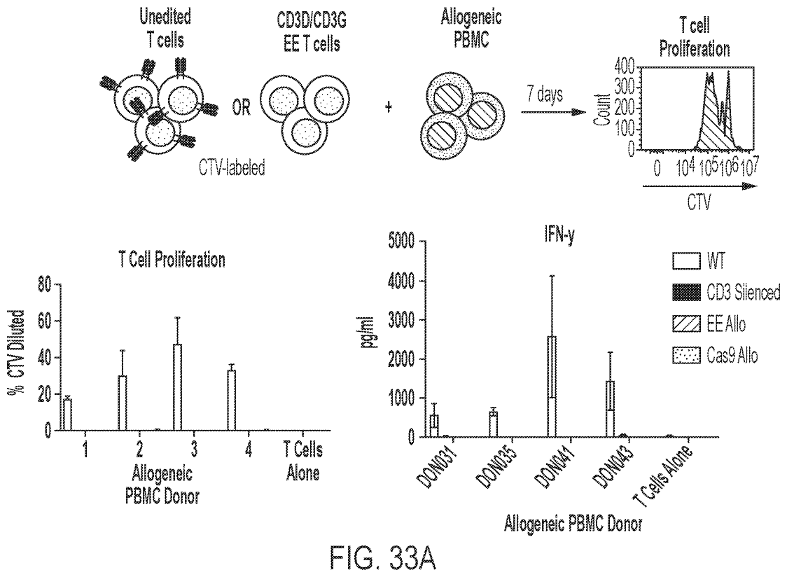

Multiplex editing of B2M, CD3D/CD3G, and RFXAP was performed in primary human T cells by current methods (Cas9 KO-wtCas9, and CRISPRi-dCas9) and compared to multiplexed epigenetic silencing using an epigenetic repressor fusion protein (CRISPR-Off) as described above by nucleofection of 2e5 activated T cells with 2.5 μg of CRISPR-Off mRNA together with a combination of gRNAs targeting the three genes (3 μg RNA138, 3 μg RNA988 2.5 μg RNA2977, 0.5 μg RNA2329, 0.5 μg RNA2510). RFXAP silencing was assessed by measuring cell surface expression of HLA-DR in the treated cell populations. Controls included wt Cas9 (positive control, Cas9 KO), epigenetic editor without guide (no guide, negative control), CRISPRi (dCas9, negative control for durability). Data shown in FIG. 32 illustrate that multiplexed, epigenetic editing achieved a comparable or significantly improved, durable reduction in B2M, CD3, and HLA-DR as compared to both wtCas9 and CRISPRi through a PMA/ionomycin restimulation at Day 14.

### Example 8: Allogeneic Function Assay Using MLR for GvHD

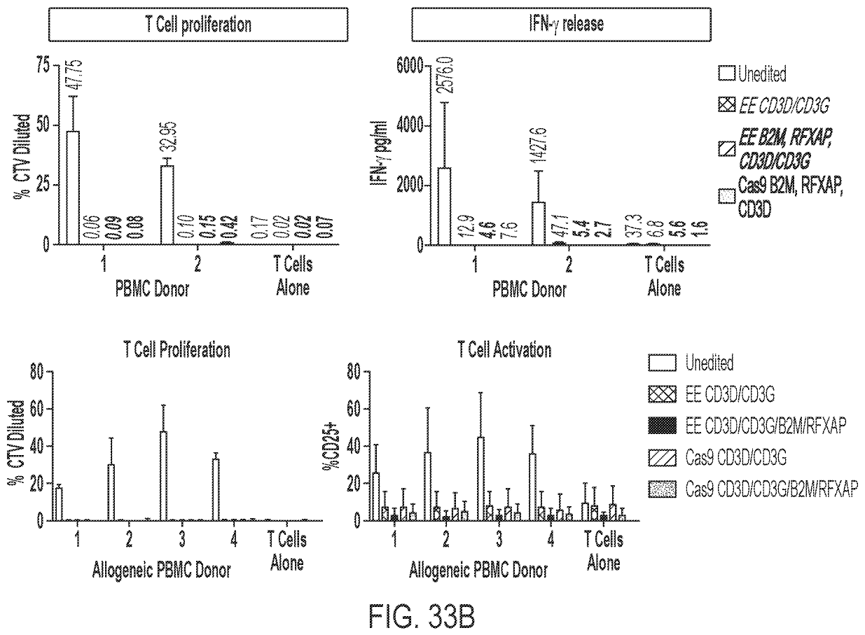

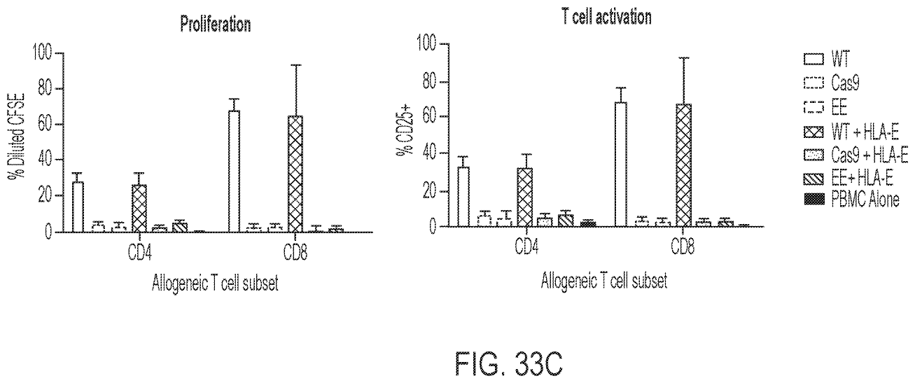

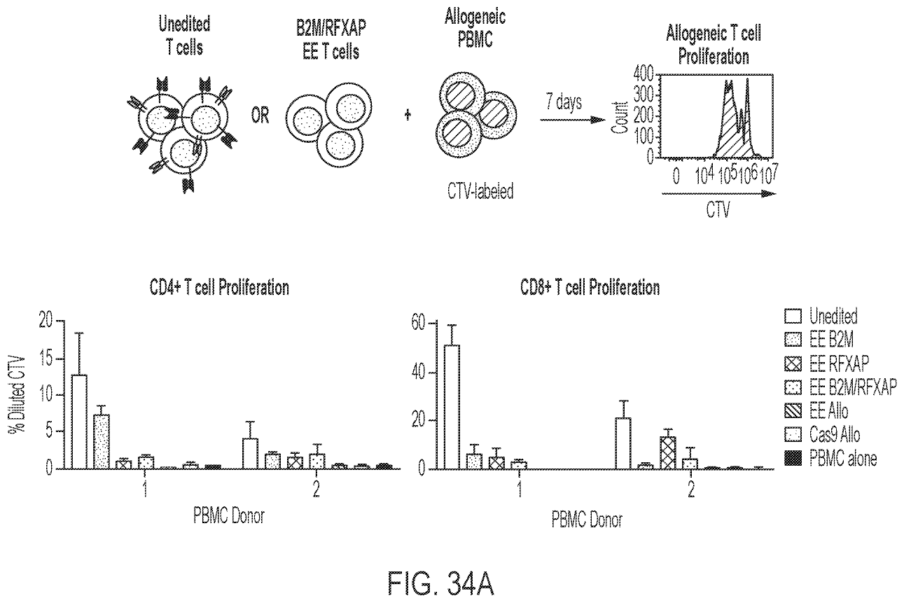

The allogeneic response by epigenetically-edited primary T cells was tested using a GvHD MLR assay (FIG. 33A, top). WT T cells (effector) were derived from a donor and epigenetically or genetically edited for the genes indicated (FIG. 33, bottom). Cells were nucleofected with 2.5 μg of CRISPR-Off mRNA together with a combination of gRNAs targeting the three genes (3 μg RNA138, 3 μg RNA949 2.5 μg RNA2977, 0.5 μg RNA2329, 0.5 μg RNA2510 per well). PBMCs were derived from another donor and used as target cells. The effector cells were labeled with 1 μM CellTrace Violet (CTV; Invitrogen, Cat. No. C34557) for 5 min at RT and combined in a 1:1 ratio with target cells and cocultured for 7 days, with supernatant collected at day 5 for assessment of cytokine release. GvHD response was evaluated by measuring proliferation of effector cells by quantifying the frequency of cells in which the CTV dye intensity was diluted by cell division (% diluted CTV). An additional measure of the GvHD response was the release of IFN-γ into the co-culture media, as assessed by Meso Scale Diagnostics assay. As shown in FIGS. 33A-33B, T cell proliferation and IFN-γ release in response to allogeneic PBMC was significantly reduced by epigenetic silencing. Cells epigenetically edited for multiple genes do not exhibit a GvHD response in vitro.

### Example 9: Allogeneic Function Assay Using MLR for Alloresponse Resistance

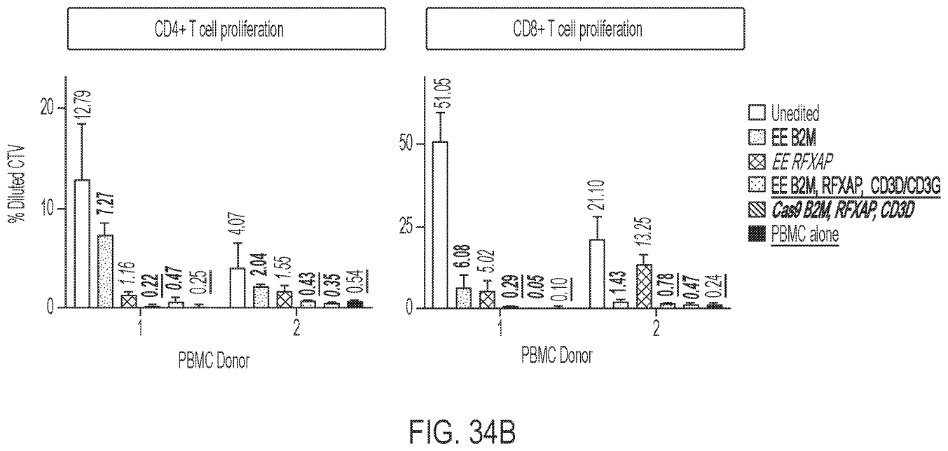

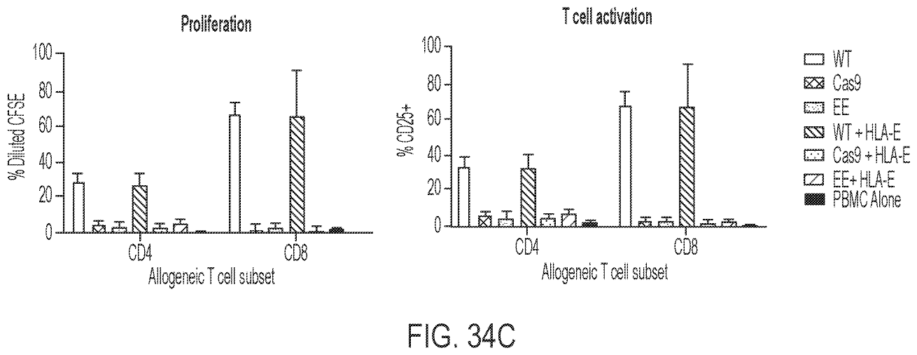

The allogeneic response to epigenetically edited primary T cells was tested using a alloresponse resistance MLR assay (FIG. 34A, top). WT T cells (target) were derived from a donor and epigenetically or genetically edited for the genes indicated (FIG. 34A, bottom; FIG. 34B). Cells were nucleofected with 2.5 μg of CRISPR-Off mRNA together with a combination of gRNAs targeting the three genes (3 μg RNA138, 3 μg RNA949 2.5 μg RNA2977, 0.5 μg RNA2329, 0.5 μg RNA2510 per well). PBMCs were derived from another donor and used as effector cells. The effector cells were labeled with 1 μM CellTrace Violet (CTV; Invitrogen, Cat. No. C34557) for 5 min at RT and combined in a 1:1 ratio with target cells and cocultured for 7 days. The alloresponse to edited T cells was evaluated by measuring proliferation of CD4+ and CD8+ T cells from the effector cell population by quantifying the frequency of cells in which the CTV dye intensity was diluted by cell division (% diluted CTV). As shown in FIG. 34, CD4+ and CD8+ T cell proliferation in response to allogeneic T cells was significantly reduced by epigenetic silencing. Cells epigenetically edited for multiple genes do not elicit a T cell alloresponse response in vitro.

### Example 10: Anti-Tumor Function of Multi-Edited Cells

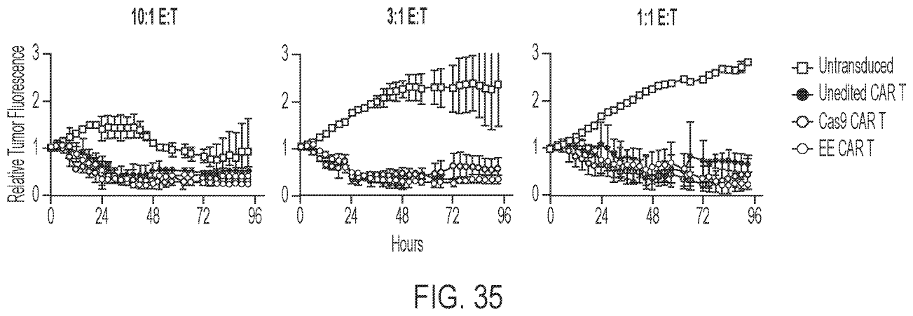

Potency of CAR T cells epigenetically silenced for B2M, CD3D/G, and RFXAP was tested in an Incucyte killing assay using MM.1S myeloma cells. Silencing of B2M, CD3D/G, and RFXAP was performed in primary human T cells by multiplexed epigenetic silencing using an epigenetic repressor fusion protein (CRISPR-Off) as described above together with a combination of gRNAs targeting the three genes (RNA101, RNA110, RNA2973, RNA2330, RNA2505). 2.5 ug mRNA, 3 ug each B2M guide, 1.5 ug RFXAP guide, and 0.5 ug each CD3 guide were used in each nucleofection well. For the killing assay, several different effector:target ratios were used, and a control sample with untransduced T cells was included. Tumor cell fluorescence intensity was used as a measure of cell survival, as the MM.1S cells were transduced to express a red fluorescent marker. The fluorescence intensity for each timepoint was normalized to the initial imaging timepoint, given as time 0 (x-axis). Images were taken every three hours for a total of six days to track CAR-mediated killing over time. A reduction in the relative tumor fluorescence (y-axis)—as indicated by values below 1—shows loss of tumor cells due to CAR T cell-mediated killing. Triple silenced CAR T have similar killing potency to unedited CAR T at three different effector:target ratios (FIG. 35).

### Example 11: In Vivo Testing of Multiplexing in an NSG MM.1S Model

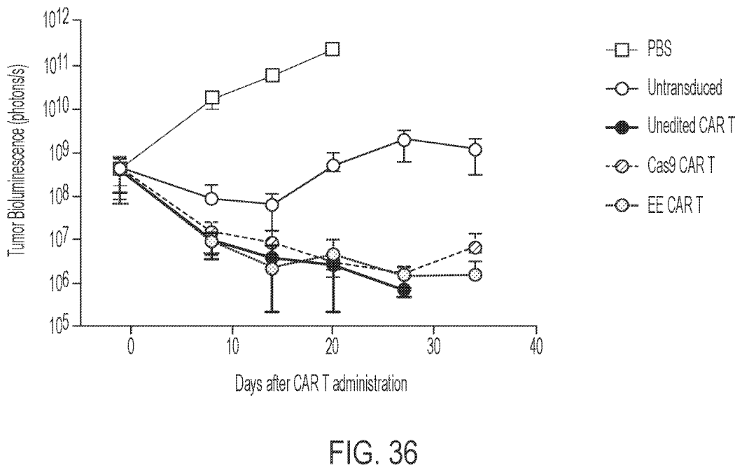

Potency of CAR T cells epigenetically silenced for B2M, CD3D/G, and RFXAP was tested in vivo in an NSG MM.1S model. Primary human T cells were transduced for expression of an anti-BCMA CAR and silencing of B2M, CD3D/G, and RFXAP was performed in transduced CAR T cells by multiplexed epigenetic silencing using an epigenetic repressor fusion protein (CRISPR-Off) as described above together with a combination of gRNAs targeting the three genes (RNA101, RNA110, RNA2973, RNA2330, RNA2505). 2.5 ug mRNA, 3 ug each B2M guide, 1.5 ug RFXAP guide, and 0.5 ug each CD3 guide were used in each nucleofection well. NSG mice were engrafted with 5e6 MM.1S-luciferase tumor cells and dosed with 5e6 CAR+ T cells intravenously 10 days later. Control mice were dosed with PBS or untransduced T cells equivalent to the total number of T cells dosed in CAR T-dosed animals. Tumor growth was monitored weekly by intraperitoneal injection of luciferin, followed by IVIS imaging weekly to quantify tumor bioluminescence. A sustained reduction in bioluminescence shows control of tumor cells by to CAR T cells. Epigenetically-edited (EE) CAR T have similar killing potency to unedited CAR T and Cas9-edited CAR T (FIG. 36). Furthermore, a GvHD response required all animals treated with unedited CAR T to be euthanized, whereas animals in the EE CAR T-treated and Cas9 CAR T-treated conditions survived longer without clinical manifestations of GvHD.

### Example 12: Anti-Tumor Function of Multi-Edited Cells

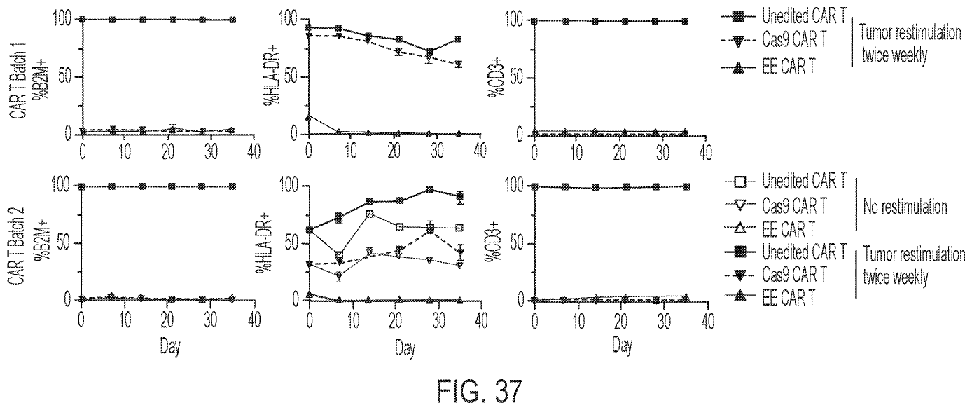

CAR T cells epigenetically silenced for B2M, CD3D/CD3G, and RFXAP were tested for sustained silencing upon repeated restimulation with tumor cells. Silencing of B2M, CD3D/CD3G, and RFXAP was performed in CAR T cells by multiplexed epigenetic silencing using an epigenetic repressor fusion protein (CRISPR-Off) together with a combination of gRNAs targeting the three genes (RNA101, RNA110, RNA2973, RNA2330, RNA2505). 2.5 ug mRNA, 3 ug each B2M guide, 1.5 ug RFXAP guide, and 0.5 ug each CD3 guide were used in each nucleofection well. RFXAP silencing was assessed by measuring cell surface expression of HLA-DR, as RFXAP is believed to be essential for HLA-DR expression. Controls included Cas9 CAR T, which were knocked out at B2M, RFXAP, and CD3D, and unedited CAR T cells. After silencing, CAR T cells were expanded and banked frozen. CAR T cells were then thawed and repeatedly exposed to tumor cells for 5 weeks post-thaw. Data shown in FIG. 37 illustrate that multiplexed, epigenetic editing achieved a durable reduction in B2M, CD3, and HLA-DR as compared to unedited CAR T through twice weekly tumor restimulation through day 35 post-thaw.

### Example 13: In Vivo Testing of Multiplexing in an NSG MM.1S Model (Lower Dose)

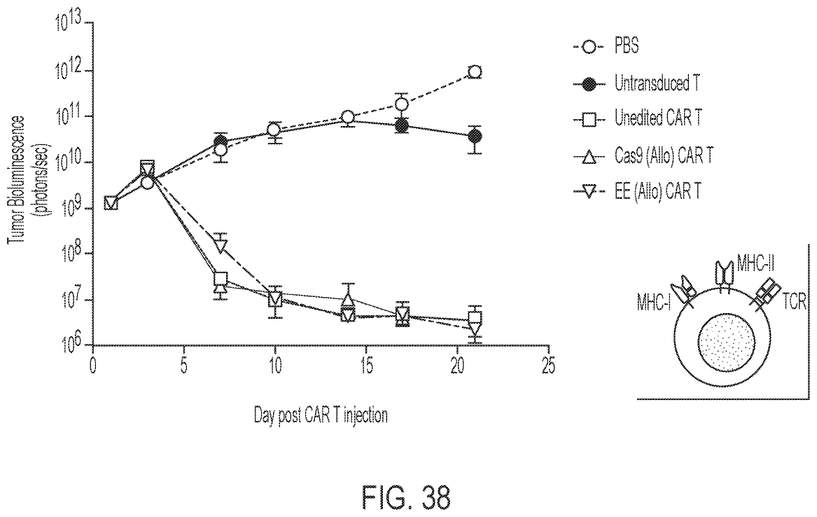

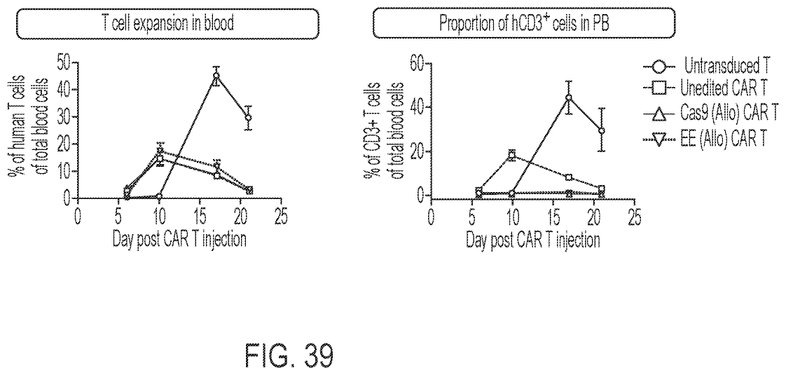

Potency of CAR T cells epigenetically silenced for B2M, CD3D/G, and RFXAP was tested in vivo in an NSG MM.1S model. Primary human T cells were transduced for expression of an anti-BCMA CAR and silencing of B2M, CD3D/G, and RFXAP was performed in transduced CAR T cells by multiplexed epigenetic silencing using an epigenetic repressor fusion protein (CRISPR-Off) as described above together with a combination of gRNAs targeting the three genes (RNA101, RNA110, RNA2973, RNA2330, RNA2505). 2.5 ug mRNA, 3 ug each B2M guide, 1.5 ug RFXAP guide, and 0.5 ug each CD3 guide were used in each nucleofection well. NSG mice were engrafted with 5e6 MM.1S-luciferase tumor cells and dosed with 1 M CAR+ T cells intravenously 10 days later. Control mice were dosed with PBS or untransduced T cells equivalent to the total number of T cells dosed in CAR T-dosed animals. Tumor growth was monitored weekly by intraperitoneal injection of luciferin, followed by IVIS imaging weekly to quantify tumor bioluminescence. A sustained reduction in bioluminescence shows control of tumor cells by to CAR T cells. Epigenetically-edited (EE) CAR T have similar killing potency to unedited CAR T and Cas9-edited CAR T (FIG. 38). Furthermore, a GvHD response required all animals treated with unedited CAR T to be euthanized, whereas animals in the EE CAR T-treated and Cas9 CAR T-treated conditions survived longer without clinical manifestations of GvHD. T cell expansion in blood and proportion of hCD3+ cells are shown in FIG. 39 (left and right respectively). Epigenetically edited CAR T cells exhibit comparable expansion in response to tumor as unedited CAR T cells and maintain silencing of CD3 target comparable to Cas9 control. Silencing is also maintained for B2M and RFXAP (data not shown).

### Example 14: Functional Assay in Primary T Cells

Multiplex silenced CAR T cells was assessed via a tumor-mediated cell killing assay.

### Method

Cytotoxicity against tumor cells in the presence of healthy donor-derived (from two different donors), B2M-RFXAP-CD3D/G-silenced or KO CAR T cells with either WT Cas9 or epigenetic editor for B2M-RFXAP-CD3D/G was assessed following co-culture with tumor cells expressing the CAR target antigen. T cells were activated and subsequently transduced with lentivirus encoding an EF1a promoter driving expression of a CAR construct. Edited CAR T cells were generated by nucleofection of the epigenetic editor mRNA and combinations of guides as unedited CAR T cells. Untransduced cells were also used as a control for non-specific killing. CAR T cells were expanded out to day 12 post-nucleofection (day 15 post-thaw) and frozen in cryopreservative media.

On the day of assay setup, CAR T cells were thawed and plated into poly-L-ornithine-coated flat-bottom 96-well plates. A set number of CAR target antigen-expressing tumor cells was added to each well. The plates were imaged using the IncuCyte to track loss of the fluorescently-labeled tumor cells over time. Dose-dependent killing was observed in all test (non-control) conditions (data not shown).

### Example 15: Selection of Optimized Guide Combinations

The below combinations of guide RNAs previously recited and tested in the Examples of this application are proposed as optimal for production of multiplex edited T cells.
1st combination: RNA2978, RNA2326, RNA2510, RNA101, and RNA110
2^{nd} combination: RNA2976, RNA2549, RNA2330, RNA102, RNA111
3^{rd} combination: RNA2973, RNA2549, RNA2330, RNA101, RNA110

### Example 16. CD3D/CD3G Methylation Profiling of Single and Multiplex Epigenetic

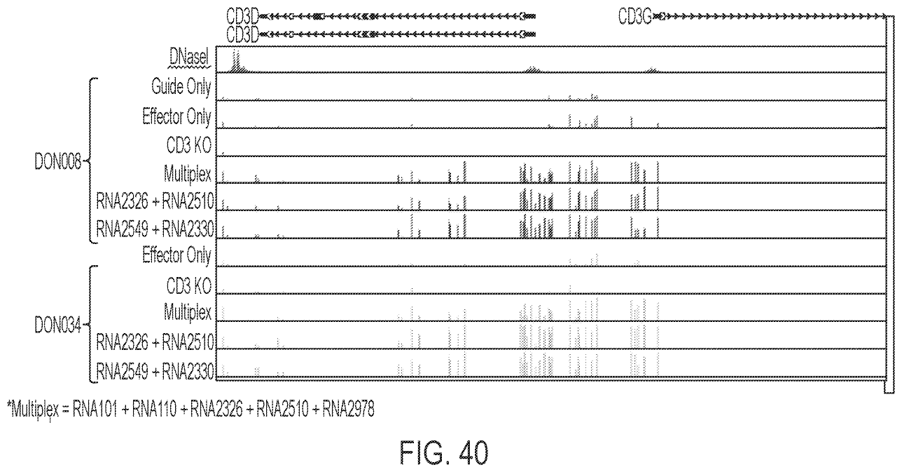

Methylation profiling of the CD3D/CD3G locus was performed in a variety of test conditions, including CD3D/CD3G knockout, CD3D/CD3G epigenetic editing, and multiplex editing (B2M, CD3D/CD3G, and RFXAP). Results are shown in FIG. 40.

### SEQUENCES

The SEQ ID NOs (SEQ) of nucleotide (nt) and amino acid (aa) sequences described in the present disclosure are listed below.

| SEQ | Description | Sequence |
|---|---|---|
| 1 | S. pyogenes WT | ATGGATAAGAAATACTCAATAGGCTTAGATATCGGCACAA |
| | Cas9 Sequence (nt) | ATAGCGTCGGATGGGCGGTGATCACTGATGAATATAAGGT |
| | | TCCGTCTAAAAAGTTCAAGGTTCTGGGAAATACAGACCGCC |
| | | ACAGTATCAAAAAAAATCTTATAGGGGCTCTTTTATTTGAC |
| | | AGTGGAGAGACAGCGGAAGCGACTCGTCTCAAACGGACAG |
| | | CTCGTAGAAGGTATACACGTCGGAAGAATCGTATTTGTTAT |
| | | CTACAGGAGATTTTTTCAAATGAGATGGCGAAAGTAGATG |
| | | ATAGTTTCTTTCATCGACTTGAAGAGTCTTTTTTGGTGGAAG |
| | | AAGACAAGAAGCATGAACGTCATCCTATTTTTGGAAATATA |
| | | GTAGATGAAGTTGCTTATCATGAGAAATATCCAACTATCTA |
| | | TCATCTGCGAAAAAAATTGGTAGATTCTACTGATAAAGCGG |
| | | ATTTGCGCTTAATCTATTTGGCCTTAGCGCATATGATTAAGT |
| | | TTCGTGGTCATTTTTTGATTGAGGGAGATTTAAATCCTGAT |
| | | AATAGTGATGTGGACAAACTATTTATCCAGTTGGTACAAAC |
| | | CTACAATCAATTATTTGAAGAAAACCCTATTAACGCAAGTG |
| | | GAGTAGATGCTAAAGCGATTCTTTCTGCACGATTGAGTAAA |
| | | TCAAGACGATTAGAAAATCTCATTGCTCAGCTCCCCGGTGA |
| | | GAAGAAAAATGGCTTATTTGGGAATCTCATTGCTTTGTCAT |
| | | TGGGTTTGACCCCTAATTTTAAATCAAATTTTGATTTGGCA |
| | | GAAGATGCTAAATTACAGCTTTCAAAAGATACTTACGATGA |
| | | TGATTTAGATAATTTATTGGCGCAAATTGGAGATCAATATG |
| | | CTGATTTGTTTTTGGCAGCTAAGAATTTATCAGATGCTATTT |
| | | TACTTTCAGATATCCTAAGAGTAAATACTGAAATAACTAAG |
| | | GCTCCCCTATCAGCTTCAATGATTAAACGCTACGATGAACA |
| | | TCATCAAGACTTGACTCTTTTAAAAGCTTTAGTTCGACAAC |
| | | AACTTCCAGAAAAGTATAAAGAAATCTTTTTTGATCAATCA |
| | | AAAAACGGATATGCAGGTTATATTGATGGGGGAGCTAGCC |
| | | AAGAAGAATTTTATAAATTTATCAAACCAATTTTAGAAAAA |
| | | ATGGATGGTACTGAGGAATTATTGGTGAAACTAAATCGTG |
| | | AAGATTTGCTGCGCAAGCAACGGACCTTTGACAACGGCTCT |
| | | ATTCCCCATCAAATTCACTTGGGTGAGCTGCATGCTATTTT |
| | | GAGAAGACAAGAAGACTTTTATCCATTTTTAAAAGACAATC |
| | | GTGAGAAGATTGAAAAAATCTTGACTTTTCGAATTCCTTAT |
| | | TATGTTGGTCCATTGGCGCGTGGCAATAGTCGTTTTGCATG |
| | | GATGACTCGGAAGTCTGAAGAAACAATTACCCCATGGAAT |
| | | TTTGAAGAAGTTGTCGATAAAGGTGCTTCAGCTCAATCATT |
| | | TATTGAACGCATGACAAACTTTGATAAAAATCTTCCAAATG |
| | | AAAAAGTACTACCAAAACATAGTTTGCTTTATGAGTATTTT |
| | | ACGGTTTATAACGAATTGACAAAGGTCAAATATGTTACTGA |
| | | AGGAATGCGAAAACCAGCATTTCTTTCAGGTGAACAGAAG |
| | | AAAGCCATTGTTGATTTACTCTTCAAAACAAATCGAAAAGT |
| | | AACCGTTAAGCAATTAAAAGAAGATTATTTCAAAAAAATA |
| | | GAATGTTTTGATAGTGTTGAAATTTCAGGAGTTGAAGATAG |
| | | ATTTAATGCTTCATTAGGTACCTACCATGATTTGCTAAAAA |
| | | TTATTAAAGATAAAGATTTTTTGGATAATGAAGAAAATGAA |
| | | GATATCTTAGAGGATATTGTTTTAACATTGACCTTATTTGA |
| | | AGATAGGGAGATGATTGAGGAAAGACTTAAAACATATGCT |
| | | CACCTCTTTGATGATAAGGTGATGAAACAGCTTAAACGTCG |
| | | CCGTTATACTGGTTGGGGACGTTTGTCTCGAAAATTGATTA |
| | | ATGGTATTAGGGATAAGCAATCTGGCAAAACAATATTAGA |
| | | TTTTTTGAAATCAGATGGTTTTGCCAATCGCAATTTTATGCA |
| | | GCTGATCCATGATGATAGTTTGACATTTAAAGAAGACATTC |
| | | AAAAAGCACAAGTGTCTGGACAAGGCGATAGTTTACATGA |
| | | ACATATTGCAAATTTAGCTGGTAGCCCTGCTATTAAAAAAG |
| | | GTATTTTACAGACTGTAAAAGTTGTTGATGAATTGGTCAAA |
| | | GTAATGGGGCGGCATAAGCCAGAAAATATCGTTATTGAAA |
| | | TGGCACGTGAAAATCAGACAACTCAAAAGGGCCAGAAAAA |
| | | TTCGCGAGAGCGTATGAAACGAATCGAAGAAGGTATCAAA |
| | | GAATTAGGAAGTCAGATTCTTAAAGAGCATCCTGTTGAAA |
| | | ATACTCAATTGCAAAATGAAAAGCTCTATCTCTATTATCTC |
| | | CAAAATGGAAGAGACATGTATGTGGACCAAGAATTAGATA |
| | | TTAATCGTTTAAGTGATTATGATGTCGATCACATTGTTCCAC |
| | | AAAGTTTCCTTAAAGACGATTCAATAGACAATAAGGTCTTA |
| | | ACGCGTTCTGATAAAAATCGTGGTAAATCGGATAACGTTCC |
| | | AAGTGAAGAAGTAGTCAAAAAGATGAAAAACTATTGGAGA |
| | | CAACTTCTAAACGCCAAGTTAATCACTCAACGTAAGTTTGA |
| | | TAATTTAACGAAAGCTGAACGTGGAGGTTTGAGTGAACTTG |
| | | ATAAAGCTGGTTTTATCAAACGCCAATTGGTTGAAACTCGC |
| | | CAAATCACTAAGCATGTGGCACAAATTTTGGATAGTCGCAT |
| | | GAATACTAAATACGATGAAAATGATAAACTTATTCGAGAG |
| | | GTTAAAGTGATTACCTTAAAATCTAAATTAGTTTCTGACTT |
| | | CCGAAAAGATTTCCAATTCTATAAAGTACGTGAGATTAACA |
| | | ATTACCATCATGCCCATGATGCGTATCTAAATGCCGTCGTT |
| | | GGAACTGCTTTGATTAAGAAATATCCAAAACTTGAATCGGA |
| | | GTTTGTCTATGGTGATTATAAAGTTTATGATGTTCGTAAAA |
| | | TGATTGCTAAGTCTGAGCAAGAAATAGGCAAAGCAACCGC |
| | | AAAATATTTCTTTTACTCTAATATCATGAACTTCTTCAAAAC |
| | | AGAAATTACACTTGCAAATGGAGAGATTCGCAAACGCCCT |
| | | CTAATCGAAACTAATGGGGAAACTGGAGAAATTGTCTGGG |
| | | ATAAAGGGCGAGATTTTGCCACAGTGCGCAAAGTATTGTCC |
| | | ATGCCCCAAGTCAATATTGTCAAGAAAACAGAAGTACAGA |
| | | CAGGCGGATTCTCCAAGGAGTCAATTTTACCAAAAAGAAA |
| | | TTCGGACAAGCTTATTGCTCGTAAAAAAGACTGGGATCCAA |
| | | AAAAATATGGTGGTTTTGATAGTCCAACGGTAGCTTATTCA |
| | | GTCCTAGTGGTTGCTAAGGTGGAAAAAGGGAAATCGAAGA |
| | | AGTTAAAATCCGTTAAAGAGTTACTAGGGATCACAATTATG |
| | | GAAAGAAGTTCCTTTGAAAAAAATCCGATTGACTTTTTAGA |
| | | AGCTAAAGGATATAAGGAAGTTAAAAAAGACTTAATCATT |
| | | AAACTACCTAAATATAGTCTTTTTGAGTTAGAAAACGGTCG |
| | | TAAACGGATGCTGGCTAGTGCCGGAGAATTACAAAAAGGA |
| | | AATGAGCTGGCTCTGCCAAGCAAATATGTGAATTTTTTATA |
| | | TTTAGCTAGTCATTATGAAAAGTTGAAGGGTAGTCCAGAAG |
| | | ATAACGAACAAAAACAATTGTTTGTGGAGCAGCATAAGCA |
| | | TTATTTAGATGAGATTATTGAGCAAATCAGTGAATTTTCTA |
| | | AGCGTGTTATTTTAGCAGATGCCAATTTAGATAAAGTTCTT |
| | | AGTGCATATAACAAACATAGAGACAAACCAATACGTGAAC |
| | | AAGCAGAAAATATTATTCATTTATTTACGTTGACGAATCTT |
| | | GGAGCTCCCGCTGCTTTTAAATATTTTGATACAACAATTGA |
| | | TCGTAAACGATATACGTCTACAAAAGAAGTTTTAGATGCCA |
| | | CTCTTATCCATCAATCCATCACTGGTCTTTATGAAACACGC |
| | | ATTGATTTGAGTCAGCTAGGAGGTGACTGA |
| 2 | S. pyogenes WT | MDKKYSIGLDIGTNSVGWAVITDEYKVPSKKFKVLGNTDRHS |
| | Cas9 Sequence (aa) | IKKNLIGALLFDSGETAEATRLKRTARRRYTRRKNRICYLQEIF |
| | | SNEMAKVDDSFFHRLEESFLVEEDKKHERHPIFGNIVDEVAYH |
| | | EKYPTIYHLRKKLVDSTDKADLRLIYLALAHMIKFRGHFLIEG |
| | | DLNPDNSDVDKLFIQLVQTYNQLFEENPINASGVDAKAILSAR |
| | | LSKSRRLENLIAQLPGEKKNGLFGNLIALSLGLTPNFKSNFDLA |
| | | EDAKLQLSKDTYDDDLDNLLAQIGDQYADLFLAAKNLSDAIL |
| | | LSDILRVNTEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFYP |
| | | FLKDNREKIEKILTFRIPYYVGPLARGNSRFAWMTRKSEETITP |
| | | WNFEEVVDKGASAQSFIERMTNFDKNLPNEKVLPKHSLLYEY |
| | | FTVYNELTKVKYVTEGMRKPAFLSGEQKKAIVDLLFKTNRKV |
| | | TVKQLKEDYFKKIECFDSVEISGVEDRFNASLGTYHDLLKIIKD |
| | | KDFLDNEENEDILEDIVLTLTLFEDREMIEERLKTYAHLFDDK |
| | | VMKQLKRRRYTGWGRLSRKLINGIRDKQSGKTILDFLKSDGF |
| | | ANRNFMQLIHDDSLTFKEDIQKAQVSGQGDSLHEHIANLAGSP |
| | | AIKKGILQTVKVVDELVKVMGRHKPENIVIEMARENQTTQKG |
| | | QKNSRERMKRIEEGIKELGSQILKEHPVENTQLQNEKLYLYYL |
| | | QNGRDMYVDQELDINRLSDYDVDHIVPQSFLKDDSIDNKVLT |
| | | RSDKNRGKSDNVPSEEVVKKMKNYWRQLLNAKLITQRKFDN |
| | | LTKAERGGLSELDKAGFIKRQLVETRQITKHVAQILDSRMNTK |
| | | YDENDKLIREVKVITLKSKLVSDFRKDFQFYKVREINNYHHA |
| | | HDAYLNAVVGTALIKKYPKLESEFVYGDYKVYDVRKMIAKS |
| | | EQEIGKATAKYFFYSNIMNFFKTEITLANGEIRKRPLIETNGET |
| | | GEIVWDKGRDFATVRKVLSMPQVNIVKKTEVQTGGFSKESIL |
| | | PKRNSDKLIARKKDWDPKKYGGFDSPTVAYSVLVVAKVEKG |
| | | KSKKLKSVKELLGITIMERSSFEKNPIDFLEAKGYKEVKKDLII |
| | | KLPKYSLFELENGRKRMLASAGELQKGNELALPSKYVNFLYL |
| | | ASHYEKLKGSPEDNEQKQLFVEQHKHYLDEIIEQISEFSKRVIL |
| | | ADANLDKVLSAYNKHRDKPIREQAENIIHLFTLTNLGAPAAFK |
| | | YFDTTIDRKRYTSTKEVLDATLIHQSITGLYETRIDLSQLGGD |
| 3 | SaCas9 | MKRNYILGLDIGITSVGYGIIDYETRDVIDAGVRLFKEANVEN |
| | | NEGRRSKRGARRLKRRRRHRIQRVKKLLFDYNLLTDHSELSGI |
| | | NPYEARVKGLSQKLSEEEFSAALLHLAKRRGVHNVNEVEEDT |
| | | GNELSTKEQISRNSKALEEKYVAELQLERLKKDGEVRGSINRF |
| | | KTSDYVKEAKQLLKVQKAYHQLDQSFIDTYIDLLETRRTYYE |
| | | GPGEGSPFGWKDIKEWYEMLMGHCTYFPEELRSVKYAYNAD |
| | | LYNALNDLNNLVITRDENEKLEYYEKFQIIENVFKQKKKPTLK |
| | | QIAKEILVNEEDIKGYRVTSTGKPEFTNLKVYHDIKDITARKEII |
| | | ENAELLDQIAKILTIYQSSEDIQEELTNLNSELTQEEIEQISNLK |
| | | GYTGTHNLSLKAINLILDELWHTNDNQIAIFNRLKLVPKKVDL |
| | | SQQKEIPTTLVDDFILSPVVKRSFIQSIKVINAIIKKYGLPNDIIIE |
| | | LAREKNSKDAQKMINEMQKRNRQTNERIEEIIRTTGKENAKY |
| | | LIEKIKLHDMQEGKCLYSLEAIPLEDLLNNPFNYEVDHIIPRSV |
| | | SFDNSFNNKVLVKQEENSKKGNRTPFQYLSSSDSKISYETFKK |
| | | HILNLAKGKGRISKTKKEYLLEERDINRFSVQKDFINRNLVDT |
| | | RYATRGLMNLLRSYFRVNNLDVKVKSINGGFTSFLRRKWKFK |
| | | KERNKGYKHHAEDALIIANADFIFKEWKKLDKAKKVMENQM |
| | | FEEKQAESMPEIETEQEYKEIFITPHQIKHIKDFKDYKYSHRVD |
| | | KKPNRELINDTLYSTRKDDKGNTLIVNNLNGLYDKDNDKLKK |
| | | LINKSPEKLLMYHHDPQTYQKLKLIMEQYGDEKNPLYKYYEE |
| | | TGNYLTKYSKKDNGPVIKKIKYYGNKLNAHLDITDDYPNSRN |
| | | KVVKLSLKPYRFDVYLDNGVYKFVTVKNLDVIKKENYYEVN |
| | | SKCYEEAKKLKKISNQAEFIASFYNNDLIKINGELYRVIGVNN |
| | | DLLNRIEVNMIDITYREYLENMNDKRPPRIIKTIASKTQSIKKYS |
| | | TDILGNLYEVKSKKHPQIIKKG |
| 4 | F. novicida WT | MSIYQEFVNKYSLSKTLRFELIPQGKTLENIKARGLILDDEKRA |
| | Cpf1 | KDYKKAKQIIDKYHQFFIEEILSSVCISEDLLQNYSDVYFKLKK |
| | | SDDDNLQKDFKSAKDTIKKQISEYIKDSEKFKNLFNQNLIDAK |
| | | KGQESDLILWLKQSKDNGIELFKANSDITDIDEALEIIKSFKGW |
| | | TTYFKGFHENRKNVYSSNDIPTSIIYRIVDDNLPKFLENKAKYE |
| | | SLKDKAPEAINYEQIKKDLAEELTFDIDYKTSEVNQRVFSLDE |
| | | VFEIANFNNYLNQSGITKFNTIIGGKFVNGENTKRKGINEYINL |
| | | YSQQINDKTLKKYKMSVLFKQILSDTESKSFVIDKLEDDSDVV |
| | | TTMQSFYEQIAAFKTVEEKSIKETLSLLFDDLKAQKLDLSKIYF |
| | | KNDKSLTDLSQQVEDDYSVIGTAVLEYITQQIAPKNLDNPSKK |
| | | EQELIAKKTEKAKYLSLETIKLALEEFNKHRDIDKQCRFEEILA |
| | | NFAAIPMIFDEIAQNKDNLAQISIKYQNQGKKDLLQASAEDDV |
| | | KAIKDLLDQTNNLLHKLKIFHISQSEDKANILDKDEHFYLVFEE |
| | | CYFELANIVPLYNKIRNYITQKPYSDEKFKLNFENSTLANGWD |
| | | KNKEPDNTAILFIKDDKYYLGVMNKKNNKIFDDKAIKENKGE |
| | | GYKKIVYKLLPGANKMLPKVFFSAKSIKFYNPSEDILRIRNHST |
| | | HTKNGSPQKGYEKFEFNIEDCRKFIDFYKQSISKHPEWKDFGF |
| | | RFSDTQRYNSIDEFYREVENQGYKLTFENISESYIDSVVNQGK |
| | | LYLFQIYNKDFSAYSKGRPNLHTLYWKALFDERNLQDVVYKL |
| | | NGEAELFYRKQSIPKKITHPAKEAIANKNKDNPKKESVFEYDL |
| | | IKDKRFTEDKFFFHCPITINFKSSGANKFNDEINLLLKEKANDV |
| | | HILSIDRGERHLAYYTLVDGKGNIIKQDTFNIIGNDRMKTNYH |
| | | DKLAAIEKDRDSARKDWKKINNIKEMKEGYLSQVVHEIAKLV |
| | | IEYNAIVVFEDLNFGFKRGRFKVEKQVYQKLEKMLIEKLNYL |
| | | VFKDNEFDKTGGVLRAYQLTAPFETFKKMGKQTGIIYYVPAG |
| | | FTSKICPVTGFVNQLYPKYESVSKSQEFFSKFDKICYNLDKGYF |
| | | EFSFDYKNFGDKAAKGKWTIASFGSRLINFRNSDKNHNWDTR |
| | | EVYPTKELEKLLKDYSIEYGHGECIKAAICGESDKKFFAKLTS |
| | | VLNTILQMRNSKTGTELDYLISPVADVNGNFFDSRQAPKNMP |
| | | QDADANGAYHIGLKGLMLLGRIKNNQEGKKLNLVIKNEEYFE |
| | | FVQNRNN |
| 5 | CasX | MEKRINKIRKKLSADNATKPVSRSGPMKTLLVRVMTDDLKKR |
| | | LEKRRKKPEVMPQVISNNAANNLRMLLDDYTKMKEAILQVY |
| | | WQEFKDDHVGLMCKFAQPASKKIDQNKLKPEMDEKGNLTTA |
| | | GFACSQCGQPLFVYKLEQVSEKGKAYTNYFGRCNVAEHEKLI |
| | | LLAQLKPEKDSDEAVTYSLGKFGQRALDFYSIHVTKESTHPVK |
| | | PLAQIAGNRYASGPVGKALSDACMGTIASFLSKYQDIIIEHQK |
| | | VVKGNQKRLESLRELAGKENLEYPSVTLPPQPHTKEGVDAYN |
| | | EVIARVRMWVNLNLWQKLKLSRDDAKPLLRLKGFPSFPVVE |
| | | RRENEVDWWNTINEVKKLIDAKRDMGRVFWSGVTAEKRNTI |
| | | LEGYNYLPNENDHKKREGSLENPKKPAKRQFGDLLLYLEKKY |
| | | AGDWGKVFDEAWERIDKKIAGLTSHIEREEARNAEDAQSKAV |
| | | LTDWLRAKASFVLERLKEMDEKEFYACEIQLQKWYGDLRGN |
| | | PFAVEAENRVVDISGFSIGSDGHSIQYRNLLAWKYLENGKREF |
| | | YLLMNYGKKGRIRFTDGTDIKKSGKWQGLLYGGGKAKVIDL |
| | | TFDPDDEQLIILPLAFGTRQGREFIWNDLLSLETGLIKLANGRVI |
| | | EKTIYNKKIGRDEPALFVALTFERREVVDPSNIKPVNLIGVDRG |
| | | ENIPAVIALTDPEGCPLPEFKDSSGGPTDILRIGEGYKEKQRAIQ |
| | | AAKEVEQRRAGGYSRKFASKSRNLADDMVRNSARDLFYHAV |
| | | THDAVLVFENLSRGFGRQGKRTFMTERQYTKMEDWLTAKLA |
| | | YEGLTSKTYLSKTLAQYTSKTCSNCGFTITTADYDGMLVRLK |
| | | KTSDGWATTLNNKELKAEGQITYYNRYKRQTVEKELSAELD |
| | | RLSEESGNNDISKWTKGRRDEALFLLKKRFSHRPVQEQFVCL |
| | | DCGHEVHADEQAALNIARSWLFLNSNSTEFKSYKSGKQPFVG |
| | | AWQAFYKRRLKEVWKPNA |
| 6 | CasY | MRKKLFKGYILHNKRLVYTGKAAIRSIKYPLVAPNKTALNNL |
| | | SEKIIYDYEHLFGPLNVASYARNSNRYSLVDFWIDSLRAGVIW |
| | | QSKSTSLIDLISKLEGSKSPSEKIFEQIDFELKNKLDKEQFKDIIL |
| | | LNTGIRSSSNVRSLRGRFLKCFKEEFRDTEEVIACVDKWSKDLI |
| | | VEGKSILVSKQFLYWEEEFGIKIFPHFKDNHDLPKLTFFVEPSL |
| | | EFSPHLPLANCLERLKKFDISRESLLGLDNNFSAFSNYFNELFN |
| | | LLSRGEIKKIVTAVLAVSKSWENEPELEKRLHFLSEKAKLLGY |
| | | PKLTSSWADYRMIIGGKIKSWHSNYTEQLIKVREDLKKHQIAL |
| | | DKLQEDLKKVVDSSLREQIEAQREALLPLLDTMLKEKDFSDD |
| | | LELYRFILSDFKSLLNGSYQRYIQTEEERKEDRDVTKKYKDLY |
| | | SNLRNIPRFFGESKKEQFNKFINKSLPTIDVGLKILEDIRNALET |
| | | VSVRKPPSITEEYVTKQLEKLSRKYKINAFNSNRFKQITEQVLR |
| | | KYNNGELPKISEVFYRYPRESHVAIRILPVKISNPRKDISYLLDK |
| | | YQISPDWKNSNPGEVVDLIEIYKLTLGWLLSCNKDFSMDFSSY |
| | | DLKLFPEAASLIKNFGSCLSGYYLSKMIFNCITSEIKGMITLYTR |
| | | DKFVVRYVTQMIGSNQKFPLLCLVGEKQTKNFSRNWGVLIEE |
| | | KGDLGEEKNQEKCLIFKDKTDFAKAKEVEIFKNNIWRIRTSKY |
| | | QIQFLNRLFKKTKEWDLMNLVLSEPSLVLEEEWGVSWDKDK |
| | | LLPLLKKEKSCEERLYYSLPLNLVPATDYKEQSAEIEQRNTYL |
| | | GLDVGEFGVAYAVVRIVRDRIELLSWGFLKDPALRKIRERVQ |
| | | DMKKKQVMAVFSSSSTAVARVREMAIHSLRNQIHSIALAYKA |
| | | KIIYEISISNFETGGNRMAKIYRSIKVSDVYRESGADTLVSEMI |
| | | WGKKNKQMGNHISSYATSYTCCNCARTPFELVIDNDKEYEK |
| | | GGDEFIFNVGDEKKVRGFLQKSLLGKTIKGKEVLKSIKEYARP |
| | | PIREVLLEGEDVEQLLKRRGNSYIYRCPFCGYKTDADIQAALN |
| | | IACRGYISDNAKDAVKEGERKLDYILEVRKLWEKNGAVLRSA |
| | | KFL |
| 7 | CasPhi | MADTPTLFTQFLRHHLPGQRFRKDILKQAGRILANKGEDATIA |
| | | FLRGKSEESPPDFQPPVKCPIIACSRPLTEWPIYQASVAIQGYV |
| | | YGQSLAEFEASDPGCSKDGLLGWFDKTGVCTDYFSVQGLNLI |
| | | FQNARKRYIGVQTKVTNRNEKRHKKLKRINAKRIAEGLPELTS |
| | | DEPESALDETGHLIDPPGLNTNIYCYQQVSPKPLALSEVNQLPT |
| | | AYAGYSTSGDDPIQPMVTKDRLSISKGQPGYIPEHQRALLSQK |
| | | KHRRMRGYGLKARALLVIVRIQDDWAVIDLRSLLRNAYWRRI |
| | | VQTKEPSTITKLLKLVTGDPVLDATRMVATFTYKPGIVQVRSA |
| | | KCLKNKQGSKLFSERYLNETVSVTSIDLGSNNLVAVATYRLV |
| | | NGNTPELLQRFTLPSHLVKDFERYKQAHDTLEDSIQKTAVASL |
| | | PQGQQTEIRMWSMYGFREAQERVCQELGLADGSIPWNVMTA |
| | | TSTILTDLFLARGGDPKKCMFTSEPKKKKNSKQVLYKIRDRA |
| | | WAKMYRTLLSKETREAWNKALWGLKRGSPDYARLSKRKEE |
| | | LARRCVNYTISTAEKRAQCGRTIVALEDLNIGFFHGRGKQEPG |
| | | WVGLFTRKKENRWLMQALHKAFLELAHHRGYHVIEVNPAYT |
| | | SQTCPVCRHCDPDNRDQHNREAFHCIGCGFRGNADLDVATH |
| | | NIAMVAITGESLKRARGSVASKTPQPLAAE |
| 8 | Cas12f1 (Cas14a) | MIKVYRYEIVKPLDLDWKEFGTILRQLQQETRFALNKATQLA |
| | | WEWMGFSSDYKDNHGEYPKSKDILGYTNVHGYAYHTIKTKA |
| | | YRLNSGNLSQTIKRATDRFKAYQKEILRGDMSIPSYKRDIPLDL |
| | | IKENISVNRMNHGDYIASLSLLSNPAKQEMNVKRKISVIIIVRG |
| | | AGKTIMDRILSGEYQVSASQIIHDDRKNKWYLNISYDFEPQTR |
| | | VLDLNKIMGIDLGVAVAVYMAFQHTPARYKLEGGEIENFRRQ |
| | | VESRRISMLRQGKYAGGARGGHGRDKRIKPIEQLRDKIANFR |
| | | DTTNHRYSRYIVDMAIKEGCGTIQMEDLTNIRDIGSRFLQNWT |
| | | YYDLQQKIIYKAEEAGIKVIKIDPQYTSQRCSECGNIDSGNRIG |
| | | QAIFKCRACGYEANADYNAARNIAIPNIDKIIAESIKSGGS |
| 9 | Cas12f2 (Cas14b) | NAMIAQKTIKIKLNPTKEQIIKLNSIIEEYIKVSNFTAKKIAEIQE |
| | | SFTDSGLTQGTCSECGKEKTYRKYHLLKKDNKLFCITCYKRK |
| | | YSQFTLQKVEFQNKTGLRNVAKLPKTYYTNAIRFASDTFSGFD |
| | | EIIKKKQNRLNSIQNRLNFWKELLYNPSNRNEIKIKVVKYAPK |
| | | TDTREHPHYYSEAEIKGRIKRLEKQLKKFKMPKYPEFTSETISL |
| | | QRELYSWKNPDELKISSITDKNESMNYYGKEYLKRYIDLINSQ |
| | | TPQILLEKENNSFYLCFPITKNIEMPKIDDTFEPVGIDWGITRNI |
| | | AVVSILDSKTKKPKFVKFYSAGYILGKRKHYKSLRKHFGQKK |
| | | RQDKINKLGTKEDRFIDSNIHKLAFLIVKEIRNHSNKPIILMENI |
| | | TDNREEAEKSMRQNILLHSVKSRLQNYIAYKALWNNIPTNLV |
| | | KPEHTSQICNRCGHQDRENRPKGSKLFKCVKCNYMSNADFN |
| | | ASINIARKFYIGEYEPFYKDNEKMKSGVNSISM |
| 10 | Cas12f3 (Cas14c) | MEVQKTVMKTLSLRILRPLYSQEIEKEIKEEEKERRKQAGGTG |
| | | ELDGGFYKKLEKKHSEMFSFDRLNLLLNQLQREIAKVYNHAI |
| | | SELYIATIAQGNKSNKHYISSIVYNRAYGYFYNAYIALGICSKV |
| | | EANFRSNELLTQQSALPTAKSDNFPIVLHKQKGAEGEDGGFRI |
| | | STEGSDLIFEIPIPFYEYNGENRKEPYKWVKKGGQKPVLKLILS |
| | | TFRRQRNKGWAKDEGTDAEIRKVTEGKYQVSQIEINRGKKLG |
| | | EHQKWFANFSIEQPIYERKPNRSIVGGLDVGIRSPLVCAINNSF |
| | | SRYSVDSNDVFKFSKQVFAFRRRLLSKNSLKRKHGHAAHKLE |
| | | PITEMTEKNDKFRKKIIERWAKEVTNFFVKNQVGIVQIEDLST |
| | | MKDREDHFFNQYLRGFWPYYQMQTLIENKLKEYGIEVKRVQ |
| | | AKYTSQLCSNPNCRYWNNYFNFEYRKVNKFPKFKCEKCNLEI |
| | | SADYNAARNLSTPDIEKFVAKATKGINLPEK |
| 11 | C2c8 | MKVLEFKIHPTEEQVSKIDQSLAACKLLWNLSIALKEESKQRY |
| | | YRKKHKFDEFSPEIWGLSYSGHYDEKEFKTLKDKEKKLLIGNP |
| | | CCKIAYFKKTSNGKEYTPLNSIPIRRFMNAENIDKDAVNYLNR |
| | | KKLAFYFRENTAKFIGEIETEFKKGFFKSVIKPAYDAAKKGIRG |
| | | IPRFKGRRDKVETLVNGQPETIKIKSNGVIVSSKIGLLKIRGLDR |
| | | LQGKAPRMAKITRKATGYYLQLTIETDDTIYKESDKCVGLDM |
| | | GAVAIFTDDLGRQSEAKRYAKIQKKRLNRLQRQASRQKDNSN |
| | | NQRKTYAKLARVHEKIARQRKGRNAQLAHKITSEYQSVILED |
| | | LNLKNMTAAAKPKEREDGDGYKQNGKKRKSGLNKALLDNAI |
| | | GQLRTFIENKANERGRKIIRVNPKHTSQTCPNCGNIDKANRVS |
| | | QSKFKCVSCGYEAHADQNAAANILIRGLRDEFLRAIGSLYKFP |
| | | VSMIGKYPGLAGEFTPDLDANQESIGDAPIENAEHSISKQMKQ |
| | | EGNRTPTQPENGSQSLIFLSAPPQPCGDSHGTNNPKALPNKAS |
| | | KRSSKKPRGAIPENPDQLTIWDLLD |
| 12 | dSpCas9 | MDKKYSIGLAIGTNSVGWAVITDEYKVPSKKFKVLGNTDRHS |
| | | IKKNLIGALLFDSGETAEATRLKRTARRRYTRRKNRICYLQEIF |
| | | SNEMAKVDDSFFHRLEESFLVEEDKKHERHPIFGNIVDEVAYH |
| | | EKYPTIYHLRKKLVDSTDKADLRLIYLALAHMIKFRGHFLIEG |
| | | DLNPDNSDVDKLFIQLVQTYNQLFEENPINASGVDAKAILSAR |
| | | LSKSRRLENLIAQLPGEKKNGLFGNLIALSLGLTPNFKSNFDLA |
| | | EDAKLQLSKDTYDDDLDNLLAQIGDQYADLFLAAKNLSDAIL |
| | | LSDILRVNTEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFYP |
| | | FLKDNREKIEKILTFRIPYYVGPLARGNSRFAWMTRKSEETITP |
| | | WNFEEVVDKGASAQSFIERMTNFDKNLPNEKVLPKHSLLYEY |
| | | FTVYNELTKVKYVTEGMRKPAFLSGEQKKAIVDLLFKTNRKV |
| | | TVKQLKEDYFKKIECFDSVEISGVEDRFNASLGTYHDLLKIIKD |
| | | KDFLDNEENEDILEDIVLTLTLFEDREMIEERLKTYAHLFDDK |
| | | VMKQLKRRRYTGWGRLSRKLINGIRDKQSGKTILDFLKSDGF |
| | | ANRNFMQLIHDDSLTFKEDIQKAQVSGQGDSLHEHIANLAGSP |
| | | AIKKGILQTVKVVDELVKVMGRHKPENIVIEMARENQTTQKG |
| | | QKNSRERMKRIEEGIKELGSQILKEHPVENTQLQNEKLYLYYL |
| | | QNGRDMYVDQELDINRLSDYDVDAIVPQSFLKDDSIDNKVLT |
| | | RSDKNRGKSDNVPSEEVVKKMKNYWRQLLNAKLITQRKFDN |
| | | LTKAERGGLSELDKAGFIKRQLVETRQITKHVAQILDSRMNTK |
| | | YDENDKLIREVKVITLKSKLVSDFRKDFQFYKVREINNYHHA |
| | | HDAYLNAVVGTALIKKYPKLESEFVYGDYKVYDVRKMIAKS |
| | | EQEIGKATAKYFFYSNIMNFFKTEITLANGEIRKRPLIETNGET |
| | | GEIVWDKGRDFATVRKVLSMPQVNIVKKTEVQTGGFSKESIL |
| | | PKRNSDKLIARKKDWDPKKYGGFDSPTVAYSVLVVAKVEKG |
| | | KSKKLKSVKELLGITIMERSSFEKNPIDFLEAKGYKEVKKDLII |
| | | KLPKYSLFELENGRKRMLASAGELQKGNELALPSKYVNFLYL |
| | | ASHYEKLKGSPEDNEQKQLFVEQHKHYLDEIIEQISEFSKRVIL |
| | | ADANLDKVLSAYNKHRDKPIREQAENIIHLFTLTNLGAPAAFK |
| | | YFDTTIDRKRYTSTKEVLDATLIHQSITGLYETRIDLSQLGGD |
| 13 | dSaCas9 | MKRNYILGLAIGITSVGYGIIDYETRDVIDAGVRLFKEANVEN |
| | | NEGRRSKRGARRLKRRRRHRIQRVKKLLFDYNLLTDHSELSGI |
| | | NPYEARVKGLSQKLSEEEFSAALLHLAKRRGVHNVNEVEEDT |
| | | GNELSTKEQISRNSKALEEKYVAELQLERLKKDGEVRGSINRF |
| | | KTSDYVKEAKQLLKVQKAYHQLDQSFIDTYIDLLETRRTYYE |
| | | GPGEGSPFGWKDIKEWYEMLMGHCTYFPEELRSVKYAYNAD |
| | | LYNALNDLNNLVITRDENEKLEYYEKFQIIENVFKQKKKPTLK |
| | | QIAKEILVNEEDIKGYRVTSTGKPEFTNLKVYHDIKDITARKEII |
| | | ENAELLDQIAKILTIYQSSEDIQEELTNLNSELTQEEIEQISNLK |
| | | GYTGTHNLSLKAINLILDELWHTNDNQIAIFNRLKLVPKKVDL |
| | | SQQKEIPTTLVDDFILSPVVKRSFIQSIKVINAIIKKYGLPNDIIIE |
| | | LAREKNSKDAQKMINEMQKRNRQTNERIEEIIRTTGKENAKY |
| | | LIEKIKLHDMQEGKCLYSLEAIPLEDLLNNPFNYEVDHIIPRSV |
| | | SFDNSFNNKVLVKQEEASKKGNRTPFQYLSSSDSKISYETFKK |
| | | HILNLAKGKGRISKTKKEYLLEERDINRFSVQKDFINRNLVDT |
| | | RYATRGLMNLLRSYFRVNNLDVKVKSINGGFTSFLRRKWKFK |
| | | KERNKGYKHHAEDALIIANADFIFKEWKKLDKAKKVMENQM |
| | | FEEKQAESMPEIETEQEYKEIFITPHQIKHIKDFKDYKYSHRVD |
| | | KKPNRELINDTLYSTRKDDKGNTLIVNNLNGLYDKDNDKLKK |
| | | LINKSPEKLLMYHHDPQTYQKLKLIMEQYGDEKNPLYKYYEE |
| | | TGNYLTKYSKKDNGPVIKKIKYYGNKLNAHLDITDDYPNSRN |
| | | KVVKLSLKPYRFDVYLDNGVYKFVTVKNLDVIKKENYYEVN |
| | | SKCYEEAKKLKKISNQAEFIASFYNNDLIKINGELYRVIGVNN |
| | | DLLNRIEVNMIDITYREYLENMNDKRPPRIIKTIASKTQSIKKYS |
| | | TDILGNLYEVKSKKHPQIIKKG |
| 14 | inactive FnCpf1 | MSIYQEFVNKYSLSKTLRFELIPQGKTLENIKARGLILDDEKRA |
| | | KDYKKAKQIIDKYHQFFIEEILSSVCISEDLLQNYSDVYFKLKK |
| | | SDDDNLQKDFKSAKDTIKKQISEYIKDSEKFKNLFNQNLIDAK |
| | | KGQESDLILWLKQSKDNGIELFKANSDITDIDEALEIIKSFKGW |
| | | TTYFKGFHENRKNVYSSNDIPTSIIYRIVDDNLPKFLENKAKYE |
| | | SLKDKAPEAINYEQIKKDLAEELTFDIDYKTSEVNQRVFSLDE |
| | | VFEIANFNNYLNQSGITKFNTIIGGKFVNGENTKRKGINEYINL |
| | | YSQQINDKTLKKYKMSVLFKQILSDTESKSFVIDKLEDDSDVV |
| | | TTMQSFYEQIAAFKTVEEKSIKETLSLLFDDLKAQKLDLSKIYF |
| | | KNDKSLTDLSQQVEDDYSVIGTAVLEYITQQIAPKNLDNPSKK |
| | | EQELIAKKTEKAKYLSLETIKLALEEFNKHRDIDKQCRFEEILA |
| | | NFAAIPMIFDEIAQNKDNLAQISIKYQNQGKKDLLQASAEDDV |
| | | KAIKDLLDQTNNLLHKLKIFHISQSEDKANILDKDEHFYLVFEE |
| | | CYFELANIVPLYNKIRNYITQKPYSDEKFKLNFENSTLANGWD |
| | | KNKEPDNTAILFIKDDKYYLGVMNKKNNKIFDDKAIKENKGE |
| | | GYKKIVYKLLPGANKMLPKVFFSAKSIKFYNPSEDILRIRNHST |
| | | HTKNGSPQKGYEKFEFNIEDCRKFIDFYKQSISKHPEWKDFGF |
| | | RFSDTQRYNSIDEFYREVENQGYKLTFENISESYIDSVVNQGK |
| | | LYLFQIYNKDFSAYSKGRPNLHTLYWKALFDERNLQDVVYKL |
| | | NGEAELFYRKQSIPKKITHPAKEAIANKNKDNPKKESVFEYDL |
| | | IKDKRFTEDKFFFHCPITINFKSSGANKENDEINLLLKEKANDV |
| | | HILSIARGERHLAYYTLVDGKGNIIKQDTFNIIGNDRMKTNYH |
| | | DKLAAIEKDRDSARKDWKKINNIKEMKEGYLSQVVHEIAKLV |
| | | IEYNAIVVFEDLNFGFKRGRFKVEKQVYQKLEKMLIEKLNYL |
| | | VFKDNEFDKTGGVLRAYQLTAPFETFKKMGKQTGIIYYVPAG |
| | | FTSKICPVTGFVNQLYPKYESVSKSQEFFSKFDKICYNLDKGYF |
| | | EFSFDYKNFGDKAAKGKWTIASFGSRLINFRNSDKNHNWDTR |
| | | EVYPTKELEKLLKDYSIEYGHGECIKAAICGESDKKFFAKLTS |
| | | VLNTILQMRNSKTGTELDYLISPVADVNGNFFDSRQAPKNMP |
| | | QDADANGAYHIGLKGLMLLGRIKNNQEGKKLNLVIKNEEYFE |
| | | FVQNRNN |
| 15 | dNmeCas9 | MAAFKPNSINYILGLAIGIASVGWAMVEIDEEENPIRLIDLGVR |
| | | VFERAEVPKTGDSLAMARRLARSVRRLTRRRAHRLLRTRRLL |
| | | KREGVLQAANFDENGLIKSLPNTPWQLRAAALDRKLTPLEWS |
| | | AVLLHLIKHRGYLSQRKNEGETADKELGALLKGVAGNAHAL |
| | | QTGDFRTPAELALNKFEKESGHIRNQRSDYSHTFSRKDLQAEL |
| | | ILLFEKQKEFGNPHVSGGLKEGIETLLMTQRPALSGDAVQKM |
| | | LGHCTFEPAEPKAAKNTYTAERFIWLTKLNNLRILEQGSERPL |
| | | TDTERATLMDEPYRKSKLTYAQARKLLGLEDTAFFKGLRYGK |
| | | DNAEASTLMEMKAYHAISRALEKEGLKDKKSPLNLSPELQDE |
| | | IGTAFSLFKTDEDITGRLKDRIQPEILEALLKHISFDKFVQISLK |
| | | ALRRIVPLMEQGKRYDEACAEIYGDHYGKKNTEEKIYLPPIPA |
| | | DEIRNPVVLRALSQARKVINGVVRRYGSPARIHIETAREVGKS |
| | | FKDRKEIEKRQEENRKDREKAAAKFREYFPNFVGEPKSKDILK |
| | | LRLYEQQHGKCLYSGKEINLGRLNEKGYVEIDAALPFSRTWD |
| | | DSFNNKVLVLGSENQNKGNQTPYEYFNGKDNSREWQEFKAR |
| | | VETSRFPRSKKQRILLQKFDEDGFKERNLNDTRYVNRFLCQFV |
| | | ADRMRLTGKGKKRVFASNGQITNLLRGFWGLRKVRAENDRH |
| | | HALDAVVVACSTVAMQQKITRFVRYKEMNAFDGKTIDKETG |
| | | EVLHQKTHFPQPWEFFAQEVMIRVFGKPDGKPEFEEADTLEK |
| | | LRTLLAEKLSSRPEAVHEYVTPLFVSRAPNRKMSGQGHMETV |
| | | KSAKRLDEGVSVLRVPLTQLKLKDLEKMVNREREPKLYEALK |
| | | ARLEAHKDDPAKAFAEPFYKYDKAGNRTQQVKAVRVEQVQ |
| | | KTGVWVRNHNGIADNATMVRVDVFEKGDKYYLVPIYSWQV |
| | | AKGILPDRAVVQGKDEEDWQLIDDSFNFKFSLHPNDLVEVITK |
| | | KARMFGYFASCHRGTGNINIRIHDLDHKIGKNGILEGIGVKTA |
| | | LSFQKYQIDELGKEIRPCRLKKRPPVR |
| 16 | dCjCas9 | MARILAFAIGISSIGWAFSENDELKDCGVRIFTKVENPKTGESL |
| | | ALPRRLARSARKRLARRKARLNHLKHLIANEFKLNYEDYQSF |
| | | DESLAKAYKGSLISPYELRFRALNELLSKQDFARVILHIAKRRG |
| | | YDDIKNSDDKEKGAILKAIKQNEEKLANYQSVGEYLYKEYFQ |
| | | KFKENSKEFTNVRNKKESYERCIAQSFLKDELKLIFKKQREFG |
| | | FSFSKKFEEEVLSVAFYKRALKDFSHLVGNCSFFTDEKRAPKN |
| | | SPLAFMFVALTRIINLLNNLKNTEGILYTKDDLNALLNEVLKN |
| | | GTLTYKQTKKLLGLSDDYEFKGEKGTYFIEFKKYKEFIKALGE |
| | | HNLSQDDLNEIAKDITLIKDEIKLKKALAKYDLNQNQIDSLSK |
| | | LEFKDHLNISFKALKLVTPLMLEGKKYDEACNELNLKVAINE |
| | | DKKDFLPAFNETYYKDEVTNPVVLRAIKEYRKVLNALLKKYG |
| | | KVHKINIELAREVGKNHSQRAKIEKEQNENYKAKKDAELECE |
| | | KLGLKINSKNILKLRLFKEQKEFCAYSGEKIKISDLQDEKMLEI |
| | | DAIYPYSRSFDDSYMNKVLVFTKQNQEKLNQTPFEAFGNDSA |
| | | KWQKIEVLAKNLPTKKQKRILDKNYKDKEQKNFKDRNLNDT |
| | | RYIARLVLNYTKDYLDFLPLSDDENTKLNDTQKGSKVHVEAK |
| | | SGMLTSALRHTWGFSAKDRNNHLHHAIDAVIIAYANNSIVKA |
| | | FSDFKKEQESNSAELYAKKISELDYKNKRKFFEPFSGFRQKVL |
| | | DKIDEIFVSKPERKKPSGALHEETFRKEEEFYQSYGGKEGVLK |
| | | ALELGKIRKVNGKIVKNGDMFRVDIFKHKKTNKFYAVPIYTM |
| | | DFALKVLPNKAVARSKKGEIKDWILMDENYEFCFSLYKDSLIL |
| | | IQTKDMQEPEFVYYNAFTSSTVSLIVSKHDNKFETLSKNQKILF |
| | | KNANEKEVIAKSIGIQNLKVFEKYIVSALGEVTKAEFRQREDF |
| | | KK |
| 17 | dSt1Cas9 | MGSDLVLGLAIGIGSVGVGILNKVTGEIIHKNSRIFPAAQAENN |
| | | LVRRTNRQGRRLARRKKHRRVRLNRLFEESGLITDFTKISINL |
| | | NPYQLRVKGLTDELSNEELFIALKNMVKHRGISYLDDASDDG |
| | | NSSVGDYAQIVKENSKQLETKTPGQIQLERYQTYGQLRGDFT |
| | | VEKDGKKHRLINVFPTSAYRSEALRILQTQQEFNPQITDEFINR |
| | | YLEILTGKRKYYHGPGNEKSRTDYGRYRTSGETLDNIFGILIG |
| | | KCTFYPDEFRAAKASYTAQEFNLLNDLNNLTVPTETKKLSKE |
| | | QKNQIINYVKNEKAMGPAKLFKYIAKLLSCDVADIKGYRIDKS |
| | | GKAEIHTFEAYRKMKTLETLDIEQMDRETLDKLAYVLTLNTE |
| | | REGIQEALEHEFADGSFSQKQVDELVQFRKANSSIFGKGWHN |
| | | FSVKLMMELIPELYETSEEQMTILTRLGKQKTTSSSNKTKYIDE |
| | | KLLTEEIYNPVVAKSVRQAIKIVNAAIKEYGDFDNIVIEMARET |
| | | NEDDEKKAIQKIQKANKDEKDAAMLKAANQYNGKAELPHSV |
| | | FHGHKQLATKIRLWHQQGERCLYTGKTISIHDLINNSNQFEVD |
| | | AILPLSITFDDSLANKVLVYATANQEKGQRTPYQALDSMDDA |
| | | WSFRELKAFVRESKTLSNKKKEYLLTEEDISKFDVRKKFIERN |
| | | LVDTRYASRVVLNALQEHFRAHKIDTKVSVVRGQFTSQLRRH |
| | | WGIEKTRDTYHHHAVDALIIAASSQLNLWKKQKNTLVSYSED |
| | | QLLDIETGELISDDEYKESVFKAPYQHFVDTLKSKEFEDSILFS |
| | | YQVDSKFNRKISDATIYATRQAKVGKDKADETYVLGKIKDIY |
| | | TQDGYDAFMKIYKKDKSKFLMYRHDPQTFEKVIEPILENYPN |
| | | KQINEKGKEVPCNPFLKYKEEHGYIRKYSKKGNGPEIKSLKYY |
| | | DSKLGNHIDITPKDSNNKVVLQSVSPWRADVYFNKTTGKYEI |
| | | LGLKYADLQFEKGTGTYKISQEKYNDIKKKEGVDSDSEFKFTL |
| | | YKNDLLLVKDTETKEQQLFRFLSRTMPKQKHYVELKPYDKQ |
| | | KFEGGEALIKVLGNVANSGQCKKGLGKSNISIYKVRTDVLGN |
| | | QHIIKNEGDKPKLDF |
| 18 | dSt3Cas9 | MTKPYSIGLAIGTNSVGWAVITDNYKVPSKKMKVLGNTSKKY |
| | | IKKNLLGVLLFDSGITAEGRRLKRTARRRYTRRRNRILYLQEIF |
| | | STEMATLDDAFFQRLDDSFLVPDDKRDSKYPIFGNLVEEKVY |
| | | HDEFPTIYHLRKYLADSTKKADLRLVYLALAHMIKYRGHFLIE |
| | | GEFNSKNNDIQKNFQDFLDTYNAIFESDLSLENSKQLEEIVKD |
| | | KISKLEKKDRILKLFPGEKNSGIFSEFLKLIVGNQADFRKCFNL |
| | | DEKASLHESKESYDEDLETLLGYIGDDYSDVFLKAKKLYDAIL |
| | | LSGFLTVTDNETEAPLSSAMIKRYNEHKEDLALLKEYIRNISLK |
| | | TYNEVFKDDTKNGYAGYIDGKTNQEDFYVYLKNLLAEFEGA |
| | | DYFLEKIDREDFLRKQRTFDNGSIPYQIHLQEMRAILDKQAKF |
| | | YPFLAKNKERIEKILTFRIPYYVGPLARGNSDFAWSIRKRNEKI |
| | | TPWNFEDVIDKESSAEAFINRMTSFDLYLPEEKVLPKHSLLYE |
| | | TFNVYNELTKVRFIAESMRDYQFLDSKQKKDIVRLYFKDKRK |
| | | VTDKDIIEYLHAIYGYDGIELKGIEKQFNSSLSTYHDLLNIINDK |
| | | EFLDDSSNEAIIEEIIHTLTIFEDREMIKQRLSKFENIFDKSVLKK |
| | | LSRRHYTGWGKLSAKLINGIRDEKSGNTILDYLIDDGISNRNF |
| | | MQLIHDDALSFKKKIQKAQIIGDEDKGNIKEVVKSLPGSPAIKK |
| | | GILQSIKIVDELVKVMGGRKPESIVVEMARENQYTNQGKSNS |
| | | QQRLKRLEKSLKELGSKILKENIPAKLSKIDNNALQNDRLYLY |
| | | YLQNGKDMYTGDDLDIDRLSNYDIDHIIPQAFLKDNSIDNKVL |
| | | VSSASARGKSDDFPSLEVVKKRKTFWYQLLKSKLISQRKFDN |
| | | LTKAERGGLLPEDKAGFIQRQLVETRQITKHVARLLDEKENN |
| | | KKDENNRAVRTVKIITLKSTLVSQFRKDFELYKVREINDFHHA |
| | | HDAYLNAVIASALLKKYPKLEPEFVYGDYPKYNSFRERKSAT |
| | | EKVYFYSNIMNIFKKSISLADGRVIERPLIEVNEETGESVWNKE |
| | | SDLATVRRVLSYPQVNVVKKVEEQNHGLDRGKPKGLFNANL |
| | | SSKPKPNSNENLVGAKEYLDPKKYGGYAGISNSFAVLVKGTIE |
| | | KGAKKKITNVLEFQGISILDRINYRKDKLNFLLEKGYKDIELIIE |
| | | LPKYSLFELSDGSRRMLASILSTNNKRGEIHKGNQIFLSQKFVK |
| | | LLYHAKRISNTINENHRKYVENHKKEFEELFYYILEFNENYVG |
| | | AKKNGKLLNSAFQSWQNHSIDELCSSFIGPTGSERKGLFELTS |
| | | RGSAADFEFLGVKIPRYRDYTPSSLLKDATLIHQSVTGLYETRI |
| | | DLAKLGEG |
| 19 | dLbCpf1 | MSKLEKFTNCYSLSKTLRFKAIPVGKTQENIDNKRLLVEDEKR |
| | | AEDYKGVKKLLDRYYLSFINDVLHSIKLKNLNNYISLFRKKTR |
| | | TEKENKELENLEINLRKEIAKAFKGNEGYKSLFKKDIIETILPEF |
| | | LDDKDEIALVNSFNGFTTAFTGFFDNRENMFSEEAKSTSIAFRC |
| | | INENLTRYISNMDIFEKVDAIFDKHEVQEIKEKILNSDYDVEDF |
| | | FEGEFFNFVLTQEGIDVYNAIIGGFVTESGEKIKGLNEYINLYN |
| | | QKTKQKLPKFKPLYKQVLSDRESLSFYGEGYTSDEEVLEVFR |
| | | NTLNKNSEIFSSIKKLEKLFKNFDEYSSAGIFVKNGPAISTISKDI |
| | | FGEWNVIRDKWNAEYDDIHLKKKAVVTEKYEDDRRKSFKKI |
| | | GSFSLEQLQEYADADLSVVEKLKEIIIQKVDEIYKVYGSSEKLF |
| | | DADFVLEKSLKKNDAVVAIMKDLLDSVKSFENYIKAFFGEGK |
| | | ETNRDESFYGDFVLAYDILLKVDHIYDAIRNYVTQKPYSKDKF |
| | | KLYFQNPQFMGGWDKDKETDYRATILRYGSKYYLAIMDKKY |
| | | AKCLQKIDKDDVNGNYEKINYKLLPGPNKMLPKVFFSKKWM |
| | | AYYNPSEDIQKIYKNGTFKKGDMFNLNDCHKLIDFFKDSISRY |
| | | PKWSNAYDFNFSETEKYKDIAGFYREVEEQGYKVSFESASKK |
| | | EVDKLVEEGKLYMFQIYNKDFSDKSHGTPNLHTMYFKLLFDE |
| | | NNHGQIRLSGGAELFMRRASLKKEELVVHPANSPIANKNPDN |
| | | PKKTTTLSYDVYKDKRFSEDQYELHIPIAINKCPKNIFKINTEV |
| | | RVLLKHDDNPYVIGIARGERNLLYIVVVDGKGNIVEQYSLNEII |
| | | NNFNGIRIKTDYHSLLDKKEKERFEARQNWTSIENIKELKAGY |
| | | ISQVVHKICELVEKYDAVIALEDLNSGFKNSRVKVEKQVYQK |
| | | FEKMLIDKLNYMVDKKSNPCATGGALKGYQITNKFESFKSMS |
| | | TQNGFIFYIPAWLTSKIDPSTGFVNLLKTKYTSIADSKKFISSFD |
| | | RIMYVPEEDLFEFALDYKNFSRTDADYIKKWKLYSYGNRIRIF |
| | | RNPKKNNVFDWEEVCLTSAYKELFNKYGINYQQGDIRALLCE |
| | | QSDKAFYSSFMALMSLMLQMRNSITGRTDVDFLISPVKNSDGI |
| | | FYDSRNYEAQENAILPKNADANGAYNIARKVLWAIGQFKKAE |
| | | DEKLDKVKIAISNKEWLEYAQTSVKH |
| 20 | inactive AsCpf1 | MTQFEGFTNLYQVSKTLRFELIPQGKTLKHIQEQGFIEEDKAR |
| | | NDHYKELKPIIDRIYKTYADQCLQLVQLDWENLSAAIDSYRKE |
| | | KTEETRNALIEEQATYRNAIHDYFIGRTDNLTDAINKRHAEIY |
| | | KGLFKAELFNGKVLKQLGTVTTTEHENALLRSFDKFTTYFSGF |
| | | YENRKNVFSAEDISTAIPHRIVQDNFPKFKENCHIFTRLITAVPS |
| | | LREHFENVKKAIGIFVSTSIEEVFSFPFYNQLLTQTQIDLYNQLL |
| | | GGISREAGTEKIKGLNEVLNLAIQKNDETAHIIASLPHRFIPLFK |
| | | QILSDRNTLSFILEEFKSDEEVIQSFCKYKTLLRNENVLETAEA |
| | | LFNELNSIDLTHIFISHKKLETISSALCDHWDTLRNALYERRISE |
| | | LTGKITKSAKEKVQRSLKHEDINLQEIISAAGKELSEAFKQKTS |
| | | EILSHAHAALDQPLPTTLKKQEEKEILKSQLDSLLGLYHLLDW |
| | | FAVDESNEVDPEFSARLTGIKLEMEPSLSFYNKARNYATKKPY |
| | | SVEKFKLNFQMPTLASGWDVNKEKNNGAILFVKNGLYYLGI |
| | | MPKQKGRYKALSFEPTEKTSEGFDKMYYDYFPDAAKMIPKCS |
| | | TQLKAVTAHFQTHTTPILLSNNFIEPLEITKEIYDLNNPEKEPKK |
| | | FQTAYAKKTGDQKGYREALCKWIDFTRDFLSKYTKTTSIDLSS |
| | | LRPSSQYKDLGEYYAELNPLLYHISFQRIAEKEIMDAVETGKL |
| | | YLFQIYNKDFAKGHHGKPNLHTLYWTGLFSPENLAKTSIKLN |
| | | GQAELFYRPKSRMKRMAHRLGEKMLNKKLKDQKTPIPDTLY |
| | | QELYDYVNHRLSHDLSDEARALLPNVITKEVSHEIIKDRRFTS |
| | | DKFFFHVPITLNYQAANSPSKFNQRVNAYLKEHPETPIIGIARG |
| | | ERNLIYITVIDSTGKILEQRSLNTIQQFDYQKKLDNREKERVAA |
| | | RQAWSVVGTIKDLKQGYLSQVIHEIVDLMIHYQAVVVLENLN |
| | | FGFKSKRTGIAEKAVYQQFEKMLIDKLNCLVLKDYPAEKVGG |
| | | VLNPYQLTDQFTSFAKMGTQSGFLFYVPAPYTSKIDPLTGFVD |
| | | PFVWKTIKNHESRKHFLEGFDFLHYDVKTGDFILHFKMNRNL |
| | | SFQRGLPGFMPAWDIVFEKNETQFDAKGTPFIAGKRIVPVIEN |
| | | HRFTGRYRDLYPANELIALLEEKGIVFRDGSNILPKLLENDDSH |
| | | AIDTMVALIRSVLQMRNSNAATGEDYINSPVRDLNGVCFDSR |
| | | FQNPEWPMDADANGAYHIALKGQLLLNHLKESKDLKLQNGI |
| | | SNQDWLAYIQELRN |
| 21 | inactive enAsCpf1 | MTQFEGFTNLYQVSKTLRFELIPQGKTLKHIQEQGFIEEDKAR |
| | | NDHYKELKPIIDRIYKTYADQCLQLVQLDWENLSAAIDSYRKE |
| | | KTEETRNALIEEQATYRNAIHDYFIGRTDNLTDAINKRHAEIY |
| | | KGLFKAELFNGKVLKQLGTVTTTEHENALLRSFDKFTTYFSGF |
| | | YRNRKNVFSAEDISTAIPHRIVQDNFPKFKENCHIFTRLITAVPS |
| | | LREHFENVKKAIGIFVSTSIEEVFSFPFYNQLLTQTQIDLYNQLL |
| | | GGISREAGTEKIKGLNEVLNLAIQKNDETAHIIASLPHRFIPLFK |
| | | QILSDRNTLSFILEEFKSDEEVIQSFCKYKTLLRNENVLETAEA |
| | | LFNELNSIDLTHIFISHKKLETISSALCDHWDTLRNALYERRISE |
| | | LTGKITKSAKEKVQRSLKHEDINLQEIISAAGKELSEAFKQKTS |
| | | EILSHAHAALDQPLPTTLKKQEEKEILKSQLDSLLGLYHLLDW |
| | | FAVDESNEVDPEFSARLTGIKLEMEPSLSFYNKARNYATKKPY |
| | | SVEKFKLNFQMPTLARGWDVNREKNNGAILFVKNGLYYLGI |
| | | MPKQKGRYKALSFEPTEKTSEGFDKMYYDYFPDAAKMIPKCS |
| | | TQLKAVTAHFQTHTTPILLSNNFIEPLEITKEIYDLNNPEKEPKK |
| | | FQTAYAKKTGDQKGYREALCKWIDFTRDFLSKYTKTTSIDLSS |
| | | LRPSSQYKDLGEYYAELNPLLYHISFQRIAEKEIMDAVETGKL |
| | | YLFQIYNKDFAKGHHGKPNLHTLYWTGLFSPENLAKTSIKLN |
| | | GQAELFYRPKSRMKRMAHRLGEKMLNKKLKDQKTPIPDTLY |
| | | QELYDYVNHRLSHDLSDEARALLPNVITKEVSHEIIKDRRFTS |
| | | DKFFFHVPITLNYQAANSPSKFNQRVNAYLKEHPETPIIGIARG |
| | | ERNLIYITVIDSTGKILEQRSLNTIQQFDYQKKLDNREKERVAA |
| | | RQAWSVVGTIKDLKQGYLSQVIHEIVDLMIHYQAVVVLENLN |
| | | FGFKSKRTGIAEKAVYQQFEKMLIDKLNCLVLKDYPAEKVGG |
| | | VLNPYQLTDQFTSFAKMGTQSGFLFYVPAPYTSKIDPLTGFVD |
| | | PFVWKTIKNHESRKHFLEGFDFLHYDVKTGDFILHFKMNRNL |
| | | SFQRGLPGFMPAWDIVFEKNETQFDAKGTPFIAGKRIVPVIEN |
| | | HRFTGRYRDLYPANELIALLEEKGIVFRDGSNILPKLLENDDSH |
| | | AIDTMVALIRSVLQMRNSNAATGEDYINSPVRDLNGVCFDSR |
| | | FQNPEWPMDADANGAYHIALKGQLLLNHLKESKDLKLQNGI |
| | | SNQDWLAYIQELRN |
| 22 | inactive HFAsCpf1 | MTQFEGFTNLYQVSKTLRFELIPQGKTLKHIQEQGFIEEDKAR |
| | | NDHYKELKPIIDRIYKTYADQCLQLVQLDWENLSAAIDSYRKE |
| | | KTEETRNALIEEQATYRNAIHDYFIGRTDNLTDAINKRHAEIY |
| | | KGLFKAELFNGKVLKQLGTVTTTEHENALLRSFDKFTTYFSGF |
| | | YRNRKNVFSAEDISTAIPHRIVQDNFPKFKENCHIFTRLITAVPS |
| | | LREHFENVKKAIGIFVSTSIEEVFSFPFYNQLLTQTQIDLYNQLL |
| | | GGISREAGTEKIKGLNEVLALAIQKNDETAHIIASLPHRFIPLFK |
| | | QILSDRNTLSFILEEFKSDEEVIQSFCKYKTLLRNENVLETAEA |
| | | LFNELNSIDLTHIFISHKKLETISSALCDHWDTLRNALYERRISE |
| | | LTGKITKSAKEKVQRSLKHEDINLQEIISAAGKELSEAFKQKTS |
| | | EILSHAHAALDQPLPTTLKKQEEKEILKSQLDSLLGLYHLLDW |
| | | FAVDESNEVDPEFSARLTGIKLEMEPSLSFYNKARNYATKKPY |
| | | SVEKFKLNFQMPTLARGWDVNREKNNGAILFVKNGLYYLGI |
| | | MPKQKGRYKALSFEPTEKTSEGFDKMYYDYFPDAAKMIPKCS |
| | | TQLKAVTAHFQTHTTPILLSNNFIEPLEITKEIYDLNNPEKEPKK |
| | | FQTAYAKKTGDQKGYREALCKWIDFTRDFLSKYTKTTSIDLSS |
| | | LRPSSQYKDLGEYYAELNPLLYHISFQRIAEKEIMDAVETGKL |
| | | YLFQIYNKDFAKGHHGKPNLHTLYWTGLFSPENLAKTSIKLN |
| | | GQAELFYRPKSRMKRMAHRLGEKMLNKKLKDQKTPIPDTLY |
| | | QELYDYVNHRLSHDLSDEARALLPNVITKEVSHEIIKDRRFTS |
| | | DKFFFHVPITLNYQAANSPSKFNQRVNAYLKEHPETPIIGIARG |
| | | ERNLIYITVIDSTGKILEQRSLNTIQQFDYQKKLDNREKERVAA |
| | | RQAWSVVGTIKDLKQGYLSQVIHEIVDLMIHYQAVVVLENLN |
| | | FGFKSKRTGIAEKAVYQQFEKMLIDKLNCLVLKDYPAEKVGG |
| | | VLNPYQLTDQFTSFAKMGTQSGFLFYVPAPYTSKIDPLTGFVD |
| | | PFVWKTIKNHESRKHFLEGFDFLHYDVKTGDFILHFKMNRNL |
| | | SFQRGLPGFMPAWDIVFEKNETQFDAKGTPFIAGKRIVPVIEN |
| | | HRFTGRYRDLYPANELIALLEEKGIVFRDGSNILPKLLENDDSH |
| | | AIDTMVALIRSVLQMRNSNAATGEDYINSPVRDLNGVCFDSR |
| | | FQNPEWPMDADANGAYHIALKGQLLLNHLKESKDLKLQNGI |
| | | SNQDWLAYIQELRN |
| 23 | inactive | MTQFEGFTNLYQVSKTLRFELIPQGKTLKHIQEQGFIEEDKAR |
| | RVRAsCpf1 | NDHYKELKPIIDRIYKTYADQCLQLVQLDWENLSAAIDSYRKE |
| | | KTEETRNALIEEQATYRNAIHDYFIGRTDNLTDAINKRHAEIY |
| | | KGLFKAELFNGKVLKQLGTVTTTEHENALLRSFDKFTTYFSGF |
| | | YENRKNVFSAEDISTAIPHRIVQDNFPKFKENCHIFTRLITAVPS |
| | | LREHFENVKKAIGIFVSTSIEEVFSFPFYNQLLTQTQIDLYNQLL |
| | | GGISREAGTEKIKGLNEVLNLAIQKNDETAHIIASLPHRFIPLFK |
| | | QILSDRNTLSFILEEFKSDEEVIQSFCKYKTLLRNENVLETAEA |
| | | LFNELNSIDLTHIFISHKKLETISSALCDHWDTLRNALYERRISE |
| | | LTGKITKSAKEKVQRSLKHEDINLQEIISAAGKELSEAFKQKTS |
| | | EILSHAHAALDQPLPTTLKKQEEKEILKSQLDSLLGLYHLLDW |
| | | FAVDESNEVDPEFSARLTGIKLEMEPSLSFYNKARNYATKKPY |
| | | SVEKFKLNFQMPTLARGWDVNVEKNRGAILFVKNGLYYLGI |
| | | MPKQKGRYKALSFEPTEKTSEGFDKMYYDYFPDAAKMIPKCS |
| | | TQLKAVTAHFQTHTTPILLSNNFIEPLEITKEIYDLNNPEKEPKK |
| | | FQTAYAKKTGDQKGYREALCKWIDFTRDFLSKYTKTTSIDLSS |
| | | LRPSSQYKDLGEYYAELNPLLYHISFQRIAEKEIMDAVETGKL |
| | | YLFQIYNKDFAKGHHGKPNLHTLYWTGLFSPENLAKTSIKLN |
| | | GQAELFYRPKSRMKRMAHRLGEKMLNKKLKDQKTPIPDTLY |
| | | QELYDYVNHRLSHDLSDEARALLPNVITKEVSHEIIKDRRFTS |
| | | DKFFFHVPITLNYQAANSPSKFNQRVNAYLKEHPETPIIGIARG |
| | | ERNLIYITVIDSTGKILEQRSLNTIQQFDYQKKLDNREKERVAA |
| | | RQAWSVVGTIKDLKQGYLSQVIHEIVDLMIHYQAVVVLENLN |
| | | FGFKSKRTGIAEKAVYQQFEKMLIDKLNCLVLKDYPAEKVGG |
| | | VLNPYQLTDQFTSFAKMGTQSGFLFYVPAPYTSKIDPLTGFVD |
| | | PFVWKTIKNHESRKHFLEGFDFLHYDVKTGDFILHFKMNRNL |
| | | SFQRGLPGFMPAWDIVFEKNETQFDAKGTPFIAGKRIVPVIEN |
| | | HRFTGRYRDLYPANELIALLEEKGIVFRDGSNILPKLLENDDSH |
| | | AIDTMVALIRSVLQMRNSNAATGEDYINSPVRDLNGVCFDSR |
| | | FQNPEWPMDADANGAYHIALKGQLLLNHLKESKDLKLQNGI |
| | | SNQDWLAYIQELRN |
| 24 | inactive | MTQFEGFTNLYQVSKTLRFELIPQGKTLKHIQEQGFIEEDKAR |
| | RRAsCpf1 | NDHYKELKPIIDRIYKTYADQCLQLVQLDWENLSAAIDSYRKE |
| | | KTEETRNALIEEQATYRNAIHDYFIGRTDNLTDAINKRHAEIY |
| | | KGLFKAELFNGKVLKQLGTVTTTEHENALLRSFDKFTTYFSGF |
| | | YENRKNVFSAEDISTAIPHRIVQDNFPKFKENCHIFTRLITAVPS |
| | | LREHFENVKKAIGIFVSTSIEEVFSFPFYNQLLTQTQIDLYNQLL |
| | | GGISREAGTEKIKGLNEVLNLAIQKNDETAHIIASLPHRFIPLFK |
| | | QILSDRNTLSFILEEFKSDEEVIQSFCKYKTLLRNENVLETAEA |
| | | LFNELNSIDLTHIFISHKKLETISSALCDHWDTLRNALYERRISE |
| | | LTGKITKSAKEKVQRSLKHEDINLQEIISAAGKELSEAFKQKTS |
| | | EILSHAHAALDQPLPTTLKKQEEKEILKSQLDSLLGLYHLLDW |
| | | FAVDESNEVDPEFSARLTGIKLEMEPSLSFYNKARNYATKKPY |
| | | SVEKFKLNFQMPTLARGWDVNKEKNNGAILFVKNGLYYLGI |
| | | MPKQKGRYKALSFEPTEKTSEGFDKMYYDYFPDAAKMIPRCS |
| | | TQLKAVTAHFQTHTTPILLSNNFIEPLEITKEIYDLNNPEKEPKK |
| | | FQTAYAKKTGDQKGYREALCKWIDFTRDFLSKYTKTTSIDLSS |
| | | LRPSSQYKDLGEYYAELNPLLYHISFQRIAEKEIMDAVETGKL |
| | | YLFQIYNKDFAKGHHGKPNLHTLYWTGLFSPENLAKTSIKLN |
| | | GQAELFYRPKSRMKRMAHRLGEKMLNKKLKDQKTPIPDTLY |
| | | QELYDYVNHRLSHDLSDEARALLPNVITKEVSHEIIKDRRFTS |
| | | DKFFFHVPITLNYQAANSPSKFNQRVNAYLKEHPETPIIGIARG |
| | | ERNLIYITVIDSTGKILEQRSLNTIQQFDYQKKLDNREKERVAA |
| | | RQAWSVVGTIKDLKQGYLSQVIHEIVDLMIHYQAVVVLENLN |
| | | FGFKSKRTGIAEKAVYQQFEKMLIDKLNCLVLKDYPAEKVGG |
| | | VLNPYQLTDQFTSFAKMGTQSGFLFYVPAPYTSKIDPLTGFVD |
| | | PFVWKTIKNHESRKHFLEGFDFLHYDVKTGDFILHFKMNRNL |
| | | SFQRGLPGFMPAWDIVFEKNETQFDAKGTPFIAGKRIVPVIEN |
| | | HRFTGRYRDLYPANELIALLEEKGIVFRDGSNILPKLLENDDSH |
| | | AIDTMVALIRSVLQMRNSNAATGEDYINSPVRDLNGVCFDSR |
| | | FQNPEWPMDADANGAYHIALKGQLLLNHLKESKDLKLQNGI |
| | | SNQDWLAYIQELRN |
| 25 | dCasX | MEKRINKIRKKLSADNATKPVSRSGPMKTLLVRVMTDDLKKR |
| | | LEKRRKKPEVMPQVISNNAANNLRMLLDDYTKMKEAILQVY |
| | | WQEFKDDHVGLMCKFAQPASKKIDQNKLKPEMDEKGNLTTA |
| | | GFACSQCGQPLFVYKLEQVSEKGKAYTNYFGRCNVAEHEKLI |
| | | LLAQLKPEKDSDEAVTYSLGKFGQRALDFYSIHVTKESTHPVK |
| | | PLAQIAGNRYASGPVGKALSDACMGTIASFLSKYQDIIIEHQK |
| | | VVKGNQKRLESLRELAGKENLEYPSVTLPPQPHTKEGVDAYN |
| | | EVIARVRMWVNLNLWQKLKLSRDDAKPLLRLKGFPSFPVVE |
| | | RRENEVDWWNTINEVKKLIDAKRDMGRVFWSGVTAEKRNTI |
| | | LEGYNYLPNENDHKKREGSLENPKKPAKRQFGDLLLYLEKKY |
| | | AGDWGKVFDEAWERIDKKIAGLTSHIEREEARNAEDAQSKAV |
| | | LTDWLRAKASFVLERLKEMDEKEFYACEIQLQKWYGDLRGN |
| | | PFAVEAENRVVDISGFSIGSDGHSIQYRNLLAWKYLENGKREF |
| | | YLLMNYGKKGRIRFTDGTDIKKSGKWQGLLYGGGKAKVIDL |
| | | TFDPDDEQLIILPLAFGTRQGREFIWNDLLSLETGLIKLANGRVI |
| | | EKTIYNKKIGRDEPALFVALTFERREVVDPSNIKPVNLIGVARG |
| | | ENIPAVIALTDPEGCPLPEFKDSSGGPTDILRIGEGYKEKQRAIQ |
| | | AAKEVEQRRAGGYSRKFASKSRNLADDMVRNSARDLFYHAV |
| | | THDAVLVFANLSRGFGRQGKRTFMTERQYTKMEDWLTAKLA |
| | | YEGLTSKTYLSKTLAQYTSKTCSNCGFTITTADYDGMLVRLK |
| | | KTSDGWATTLNNKELKAEGQITYYNRYKRQTVEKELSAELD |
| | | RLSEESGNNDISKWTKGRRDEALFLLKKRFSHRPVQEQFVCL |
| | | DCGHEVHAAEQAALNIARSWLFLNSNSTEFKSYKSGKQPFVG |
| | | AWQAFYKRRLKEVWKPNA |
| 26 | dCasPhi | MPKPAVESEFSKVLKKHFPGERFRSSYMKRGGKILAAQGEEA |
| | | VVAYLQGKSEEEPPNFQPPAKCHVVTKSRDFAEWPIMKASEA |
| | | IQRYIYALSTTERAACKPGKSSESHAAWFAATGVSNHGYSHV |
| | | QGLNLIFDHTLGRYDGVLKKVQLRNEKARARLESINASRADE |
| | | GLPEIKAEEEEVATNETGHLLQPPGINPSFYVYQTISPQAYRPR |
| | | DEIVLPPEYAGYVRDPNAPIPLGVVRNRCDIQKGCPGYIPEWQ |
| | | REAGTAISPKTGKAVTVPGLSPKKNKRMRRYWRSEKEKAQD |
| | | ALLVTVRIGTDWVVIDVRGLLRNARWRTIAPKDISLNALLDLF |
| | | TGDPVIDVRRNIVTFTYTLDACGTYARKWTLKGKQTKATLDK |
| | | LTATQTVALVAIALGQTNPISAGISRVTQENGALQCEPLDRFTL |
| | | PDDLLKDISAYRIAWDRNEEELRARSVEALPEAQQAEVRALD |
| | | GVSKETARTQLCADFGLDPKRLPWDKMSSNTTFISEALLSNSV |
| | | SRDQVFFTPAPKKGAKKKAPVEVMRKDRTWARAYKPRLSVE |
| | | AQKLKNEALWALKRTSPEYLKLSRRKEELCRRSINYVIEKTRR |
| | | RTQCQIVIPVIEDLNVRFFHGSGKRLPGWDNFFTAKKENRWFI |
| | | QGLHKAFSDLRTHRSFYVFEVRPERTSITCPKCGHCEVGNRDG |
| | | EAFQCLSCGKTCNADLDVATHNLTQVALTGKTMPKREEPRD |
| | | AQGTAPARKTKKASKSKAPPAEREDQTPAQEPSQTS |
| 27 | inactive VRER | MDKKYSIGLAIGTNSVGWAVITDEYKVPSKKFKVLGNTDRHS |
| | SpCas9 | IKKNLIGALLFDSGETAEATRLKRTARRRYTRRKNRICYLQEIF |
| | | SNEMAKVDDSFFHRLEESFLVEEDKKHERHPIFGNIVDEVAYH |
| | | EKYPTIYHLRKKLVDSTDKADLRLIYLALAHMIKFRGHFLIEG |
| | | DLNPDNSDVDKLFIQLVQTYNQLFEENPINASGVDAKAILSAR |
| | | LSKSRRLENLIAQLPGEKKNGLFGNLIALSLGLTPNFKSNFDLA |
| | | EDAKLQLSKDTYDDDLDNLLAQIGDQYADLFLAAKNLSDAIL |
| | | LSDILRVNTEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFYP |
| | | FLKDNREKIEKILTFRIPYYVGPLARGNSRFAWMTRKSEETITP |
| | | WNFEEVVDKGASAQSFIERMTNFDKNLPNEKVLPKHSLLYEY |
| | | FTVYNELTKVKYVTEGMRKPAFLSGEQKKAIVDLLFKTNRKV |
| | | TVKQLKEDYFKKIECFDSVEISGVEDRFNASLGTYHDLLKIIKD |
| | | KDFLDNEENEDILEDIVLTLTLFEDREMIEERLKTYAHLFDDK |
| | | VMKQLKRRRYTGWGRLSRKLINGIRDKQSGKTILDFLKSDGF |
| | | ANRNFMQLIHDDSLTFKEDIQKAQVSGQGDSLHEHIANLAGSP |
| | | AIKKGILQTVKVVDELVKVMGRHKPENIVIEMARENQTTQKG |
| | | QKNSRERMKRIEEGIKELGSQILKEHPVENTQLQNEKLYLYYL |
| | | QNGRDMYVDQELDINRLSDYDVDAIVPQSFLKDDSIDNKVLT |
| | | RSDKNRGKSDNVPSEEVVKKMKNYWRQLLNAKLITQRKFDN |
| | | LTKAERGGLSELDKAGFIKRQLVETRQITKHVAQILDSRMNTK |
| | | YDENDKLIREVKVITLKSKLVSDFRKDFQFYKVREINNYHHA |
| | | HDAYLNAVVGTALIKKYPKLESEFVYGDYKVYDVRKMIAKS |
| | | EQEIGKATAKYFFYSNIMNFFKTEITLANGEIRKRPLIETNGET |
| | | GEIVWDKGRDFATVRKVLSMPQVNIVKKTEVQTGGFSKESIL |
| | | PKRNSDKLIARKKDWDPKKYGGFVSPTVAYSVLVVAKVEKG |
| | | KSKKLKSVKELLGITIMERSSFEKNPIDFLEAKGYKEVKKDLII |
| | | KLPKYSLFELENGRKRMLASARELQKGNELALPSKYVNFLYL |
| | | ASHYEKLKGSPEDNEQKQLFVEQHKHYLDEIIEQISEFSKRVIL |
| | | ADANLDKVLSAYNKHRDKPIREQAENIIHLFTLTNLGAPAAFK |
| | | YFDTTIDRKEYRSTKEVLDATLIHQSITGLYETRIDLSQLGGD |
| 28 | inactive EQR | MDKKYSIGLAIGTNSVGWAVITDEYKVPSKKFKVLGNTDRHS |
| | SpCas9 | IKKNLIGALLFDSGETAEATRLKRTARRRYTRRKNRICYLQEIF |
| | | SNEMAKVDDSFFHRLEESFLVEEDKKHERHPIFGNIVDEVAYH |
| | | EKYPTIYHLRKKLVDSTDKADLRLIYLALAHMIKFRGHFLIEG |
| | | DLNPDNSDVDKLFIQLVQTYNQLFEENPINASGVDAKAILSAR |
| | | LSKSRRLENLIAQLPGEKKNGLFGNLIALSLGLTPNFKSNFDLA |
| | | EDAKLQLSKDTYDDDLDNLLAQIGDQYADLFLAAKNLSDAIL |
| | | LSDILRVNTEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFYP |
| | | FLKDNREKIEKILTFRIPYYVGPLARGNSRFAWMTRKSEETITP |
| | | WNFEEVVDKGASAQSFIERMTNFDKNLPNEKVLPKHSLLYEY |
| | | FTVYNELTKVKYVTEGMRKPAFLSGEQKKAIVDLLFKTNRKV |
| | | TVKQLKEDYFKKIECFDSVEISGVEDRFNASLGTYHDLLKIIKD |
| | | KDFLDNEENEDILEDIVLTLTLFEDREMIEERLKTYAHLFDDK |
| | | VMKQLKRRRYTGWGRLSRKLINGIRDKQSGKTILDFLKSDGF |
| | | ANRNFMQLIHDDSLTFKEDIQKAQVSGQGDSLHEHIANLAGSP |
| | | AIKKGILQTVKVVDELVKVMGRHKPENIVIEMARENQTTQKG |
| | | QKNSRERMKRIEEGIKELGSQILKEHPVENTQLQNEKLYLYYL |
| | | QNGRDMYVDQELDINRLSDYDVDAIVPQSFLKDDSIDNKVLT |
| | | RSDKNRGKSDNVPSEEVVKKMKNYWRQLLNAKLITQRKFDN |
| | | LTKAERGGLSELDKAGFIKRQLVETRQITKHVAQILDSRMNTK |
| | | YDENDKLIREVKVITLKSKLVSDFRKDFQFYKVREINNYHHA |
| | | HDAYLNAVVGTALIKKYPKLESEFVYGDYKVYDVRKMIAKS |
| | | EQEIGKATAKYFFYSNIMNFFKTEITLANGEIRKRPLIETNGET |
| | | GEIVWDKGRDFATVRKVLSMPQVNIVKKTEVQTGGFSKESIL |
| | | PKRNSDKLIARKKDWDPKKYGGFESPTVAYSVLVVAKVEKG |
| | | KSKKLKSVKELLGITIMERSSFEKNPIDFLEAKGYKEVKKDLII |
| | | KLPKYSLFELENGRKRMLASAGELQKGNELALPSKYVNFLYL |
| | | ASHYEKLKGSPEDNEQKQLFVEQHKHYLDEIIEQISEFSKRVIL |
| | | ADANLDKVLSAYNKHRDKPIREQAENIIHLFTLTNLGAPAAFK |
| | | YFDTTIDRKQYRSTKEVLDATLIHQSITGLYETRIDLSQLGGD |
| 29 | inactive VQR | MDKKYSIGLAIGTNSVGWAVITDEYKVPSKKFKVLGNTDRHS |
| | SpCas9 | IKKNLIGALLFDSGETAEATRLKRTARRRYTRRKNRICYLQEIF |
| | | SNEMAKVDDSFFHRLEESFLVEEDKKHERHPIFGNIVDEVAYH |
| | | EKYPTIYHLRKKLVDSTDKADLRLIYLALAHMIKFRGHFLIEG |
| | | DLNPDNSDVDKLFIQLVQTYNQLFEENPINASGVDAKAILSAR |
| | | LSKSRRLENLIAQLPGEKKNGLFGNLIALSLGLTPNFKSNFDLA |
| | | EDAKLQLSKDTYDDDLDNLLAQIGDQYADLFLAAKNLSDAIL |
| | | LSDILRVNTEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFYP |
| | | FLKDNREKIEKILTFRIPYYVGPLARGNSRFAWMTRKSEETITP |
| | | WNFEEVVDKGASAQSFIERMTNFDKNLPNEKVLPKHSLLYEY |
| | | FTVYNELTKVKYVTEGMRKPAFLSGEQKKAIVDLLFKTNRKV |
| | | TVKQLKEDYFKKIECFDSVEISGVEDRFNASLGTYHDLLKIIKD |
| | | KDFLDNEENEDILEDIVLTLTLFEDREMIEERLKTYAHLFDDK |
| | | VMKQLKRRRYTGWGRLSRKLINGIRDKQSGKTILDFLKSDGF |
| | | ANRNFMQLIHDDSLTFKEDIQKAQVSGQGDSLHEHIANLAGSP |
| | | AIKKGILQTVKVVDELVKVMGRHKPENIVIEMARENQTTQKG |
| | | QKNSRERMKRIEEGIKELGSQILKEHPVENTQLQNEKLYLYYL |
| | | QNGRDMYVDQELDINRLSDYDVDAIVPQSFLKDDSIDNKVLT |
| | | RSDKNRGKSDNVPSEEVVKKMKNYWRQLLNAKLITQRKFDN |
| | | LTKAERGGLSELDKAGFIKRQLVETRQITKHVAQILDSRMNTK |
| | | YDENDKLIREVKVITLKSKLVSDFRKDFQFYKVREINNYHHA |
| | | HDAYLNAVVGTALIKKYPKLESEFVYGDYKVYDVRKMIAKS |
| | | EQEIGKATAKYFFYSNIMNFFKTEITLANGEIRKRPLIETNGET |
| | | GEIVWDKGRDFATVRKVLSMPQVNIVKKTEVQTGGFSKESIL |
| | | PKRNSDKLIARKKDWDPKKYGGFVSPTVAYSVLVVAKVEKG |
| | | KSKKLKSVKELLGITIMERSSFEKNPIDFLEAKGYKEVKKDLII |
| | | KLPKYSLFELENGRKRMLASAGELQKGNELALPSKYVNFLYL |
| | | ASHYEKLKGSPEDNEQKQLFVEQHKHYLDEIIEQISEFSKRVIL |
| | | ADANLDKVLSAYNKHRDKPIREQAENIIHLFTLTNLGAPAAFK |
| | | YFDTTIDRKQYRSTKEVLDATLIHQSITGLYETRIDLSQLGGD |
| 30 | inactive SPG | MDKKYSIGLAIGTNSVGWAVITDEYKVPSKKFKVLGNTDRHS |
| | SpCas9 | IKKNLIGALLFDSGETAEATRLKRTARRRYTRRKNRICYLQEIF |
| | | SNEMAKVDDSFFHRLEESFLVEEDKKHERHPIFGNIVDEVAYH |
| | | EKYPTIYHLRKKLVDSTDKADLRLIYLALAHMIKFRGHFLIEG |
| | | DLNPDNSDVDKLFIQLVQTYNQLFEENPINASGVDAKAILSAR |
| | | LSKSRRLENLIAQLPGEKKNGLFGNLIALSLGLTPNFKSNFDLA |
| | | EDAKLQLSKDTYDDDLDNLLAQIGDQYADLFLAAKNLSDAIL |
| | | LSDILRVNTEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFYP |
| | | FLKDNREKIEKILTFRIPYYVGPLARGNSRFAWMTRKSEETITP |
| | | WNFEEVVDKGASAQSFIERMTNFDKNLPNEKVLPKHSLLYEY |
| | | FTVYNELTKVKYVTEGMRKPAFLSGEQKKAIVDLLFKTNRKV |
| | | TVKQLKEDYFKKIECFDSVEISGVEDRFNASLGTYHDLLKIIKD |
| | | KDFLDNEENEDILEDIVLTLTLFEDREMIEERLKTYAHLFDDK |
| | | VMKQLKRRRYTGWGRLSRKLINGIRDKQSGKTILDFLKSDGF |
| | | ANRNFMQLIHDDSLTFKEDIQKAQVSGQGDSLHEHIANLAGSP |
| | | AIKKGILQTVKVVDELVKVMGRHKPENIVIEMARENQTTQKG |
| | | QKNSRERMKRIEEGIKELGSQILKEHPVENTQLQNEKLYLYYL |
| | | QNGRDMYVDQELDINRLSDYDVDAIVPQSFLKDDSIDNKVLT |
| | | RSDKNRGKSDNVPSEEVVKKMKNYWRQLLNAKLITQRKFDN |
| | | LTKAERGGLSELDKAGFIKRQLVETRQITKHVAQILDSRMNTK |
| | | YDENDKLIREVKVITLKSKLVSDFRKDFQFYKVREINNYHHA |
| | | HDAYLNAVVGTALIKKYPKLESEFVYGDYKVYDVRKMIAKS |
| | | EQEIGKATAKYFFYSNIMNFFKTEITLANGEIRKRPLIETNGET |
| | | GEIVWDKGRDFATVRKVLSMPQVNIVKKTEVQTGGFSKESIL |
| | | PKRNSDKLIARKKDWDPKKYGGFLWPTVAYSVLVVAKVEKG |
| | | KSKKLKSVKELLGITIMERSSFEKNPIDFLEAKGYKEVKKDLII |
| | | KLPKYSLFELENGRKRMLASAKQLQKGNELALPSKYVNFLYL |
| | | ASHYEKLKGSPEDNEQKQLFVEQHKHYLDEIIEQISEFSKRVIL |
| | | ADANLDKVLSAYNKHRDKPIREQAENIIHLFTLTNLGAPAAFK |
| | | YFDTTIDRKQYRSTKEVLDATLIHQSITGLYETRIDLSQLGGD |
| 31 | inactive SpRY | MDKKYSIGLAIGTNSVGWAVITDEYKVPSKKFKVLGNTDRHS |
| | Cas9 | IKKNLIGALLFDSGETAERTRLKRTARRRYTRRKNRICYLQEIF |
| | | SNEMAKVDDSFFHRLEESFLVEEDKKHERHPIFGNIVDEVAYH |
| | | EKYPTIYHLRKKLVDSTDKADLRLIYLALAHMIKFRGHFLIEG |
| | | DLNPDNSDVDKLFIQLVQTYNQLFEENPINASGVDAKAILSAR |
| | | LSKSRRLENLIAQLPGEKKNGLFGNLIALSLGLTPNFKSNFDLA |
| | | EDAKLQLSKDTYDDDLDNLLAQIGDQYADLFLAAKNLSDAIL |
| | | LSDILRVNTEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFYP |
| | | FLKDNREKIEKILTFRIPYYVGPLARGNSRFAWMTRKSEETITP |
| | | WNFEEVVDKGASAQSFIERMTNFDKNLPNEKVLPKHSLLYEY |
| | | FTVYNELTKVKYVTEGMRKPAFLSGEQKKAIVDLLFKTNRKV |
| | | TVKQLKEDYFKKIECFDSVEISGVEDRFNASLGTYHDLLKIIKD |
| | | KDFLDNEENEDILEDIVLTLTLFEDREMIEERLKTYAHLFDDK |
| | | VMKQLKRRRYTGWGRLSRKLINGIRDKQSGKTILDFLKSDGF |
| | | ANRNFMQLIHDDSLTFKEDIQKAQVSGQGDSLHEHIANLAGSP |
| | | AIKKGILQTVKVVDELVKVMGRHKPENIVIEMARENQTTQKG |
| | | QKNSRERMKRIEEGIKELGSQILKEHPVENTQLQNEKLYLYYL |
| | | QNGRDMYVDQELDINRLSDYDVDAIVPQSFLKDDSIDNKVLT |
| | | RSDKNRGKSDNVPSEEVVKKMKNYWRQLLNAKLITQRKFDN |
| | | LTKAERGGLSELDKAGFIKRQLVETRQITKHVAQILDSRMNTK |
| | | YDENDKLIREVKVITLKSKLVSDFRKDFQFYKVREINNYHHA |
| | | HDAYLNAVVGTALIKKYPKLESEFVYGDYKVYDVRKMIAKS |
| | | EQEIGKATAKYFFYSNIMNFFKTEITLANGEIRKRPLIETNGET |
| | | GEIVWDKGRDFATVRKVLSMPQVNIVKKTEVQTGGFSKESIR |
| | | PKRNSDKLIARKKDWDPKKYGGFLWPTVAYSVLVVAKVEKG |
| | | KSKKLKSVKELLGITIMERSSFEKNPIDFLEAKGYKEVKKDLII |
| | | KLPKYSLFELENGRKRMLASAKQLQKGNELALPSKYVNFLYL |
| | | ASHYEKLKGSPEDNEQKQLFVEQHKHYLDEIIEQISEFSKRVIL |
| | | ADANLDKVLSAYNKHRDKPIREQAENIIHLFTLTRLGAPRAFK |
| | | YFDTTIDPKQYRSTKEVLDATLIHQSITGLYETRIDLSQLGGD |
| 32 | inactive KKH | MKRNYILGLAIGITSVGYGIIDYETRDVIDAGVRLFKEANVEN |
| | dSaCas9 | NEGRRSKRGARRLKRRRRHRIQRVKKLLFDYNLLTDHSELSGI |
| | | NPYEARVKGLSQKLSEEEFSAALLHLAKRRGVHNVNEVEEDT |
| | | GNELSTKEQISRNSKALEEKYVAELQLERLKKDGEVRGSINRF |
| | | KTSDYVKEAKQLLKVQKAYHQLDQSFIDTYIDLLETRRTYYE |
| | | GPGEGSPFGWKDIKEWYEMLMGHCTYFPEELRSVKYAYNAD |
| | | LYNALNDLNNLVITRDENEKLEYYEKFQIIENVFKQKKKPTLK |
| | | QIAKEILVNEEDIKGYRVTSTGKPEFTNLKVYHDIKDITARKEII |
| | | ENAELLDQIAKILTIYQSSEDIQEELTNLNSELTQEEIEQISNLK |
| | | GYTGTHNLSLKAINLILDELWHTNDNQIAIFNRLKLVPKKVDL |
| | | SQQKEIPTTLVDDFILSPVVKRSFIQSIKVINAIIKKYGLPNDIIIE |
| | | LAREKNSKDAQKMINEMQKRNRQTNERIEEIIRTTGKENAKY |
| | | LIEKIKLHDMQEGKCLYSLEAIPLEDLLNNPFNYEVDHIIPRSV |
| | | SFDNSFNNKVLVKQEEASKKGNRTPFQYLSSSDSKISYETFKK |
| | | HILNLAKGKGRISKTKKEYLLEERDINRFSVQKDFINRNLVDT |
| | | RYATRGLMNLLRSYFRVNNLDVKVKSINGGFTSFLRRKWKFK |
| | | KERNKGYKHHAEDALIIANADFIFKEWKKLDKAKKVMENQM |
| | | FEEKQAESMPEIETEQEYKEIFITPHQIKHIKDFKDYKYSHRVD |
| | | KKPNRKLINDTLYSTRKDDKGNTLIVNNLNGLYDKDNDKLK |
| | | KLINKSPEKLLMYHHDPQTYQKLKLIMEQYGDEKNPLYKYYE |
| | | ETGNYLTKYSKKDNGPVIKKIKYYGNKLNAHLDITDDYPNSR |
| | | NKVVKLSLKPYRFDVYLDNGVYKFVTVKNLDVIKKENYYEV |
| | | NSKCYEEAKKLKKISNQAEFIASFYKNDLIKINGELYRVIGVN |
| | | NDLLNRIEVNMIDITYREYLENMNDKRPPHIIKTIASKTQSIKK |
| | | YSTDILGNLYEVKSKKHPQIIKKG |
| 33 | ZIM3 | MNNSQGRVTFEDVTVNFTQGEWQRLNPEQRNLYRDVMLEN |
| | | YSNLVSVGQGETTKPDVILRLEQGKEPWLEEEEVLGSGRAEK |
| | | NGDIGGQIWKPKDVKESL |
| 34 | ZNF436 | MAATLLMAGSQAPVTFEDMAMYLTREEWRPLDAAQRDLYR |
| | | DVMQENYGNVVSLDFEIRSENEVNPKQEISEDVQFGTTSERPA |
| | | ENAEENPESEEGFESGDRSERQW |
| 35 | ZNF257 | MLENYRNLVFLGIAVSKPDLITCLEQGKEPCNMKRHEMVAKP |
| | | PVMCSHIAEDLCPERDIKYFFQKVILRRYDKCEHENLQLRKGC |
| | | KSVDECKVCK |
| 36 | ZNF675 | MGLLTFRDVAIEFSLEEWQCLDTAQRNLYKNVILENYRNLVF |
| | | LGIAVSKQDLITCLEQEKEPLTVKRHEMVNEPPVMCSHFAQEF |
| | | WPEQNIKDSF |
| 37 | ZNF490 | MLQMQNSEHHGQSIKTQTDSISLEDVAVNFTLEEWALLDPGQ |
| | | RNIYRDVMRATFKNLACIGEKWKDQDIEDEHKNQGRNLRSP |
| | | MVEALCENKEDCPCGKSTSQIPDLNTNLETPTG |
| 38 | ZNF320 | MALSQGLLTFRDVAIEFSQEEWKCLDPAQRTLYRDVMLENYR |
| | | NLVSLDISSKCMMNTLSSTGQGNTEVIHTGTLQRQASYHIGAF |
| | | CSQEIEKDIHDFVFQ |
| 39 | ZNF331 | MAQGLVTFADVAIDFSQEEWACLNSAQRDLYWDVMLENYS |
| | | NLVSLDLESAYENKSLPTKKNIHEIRASKRNSDRRSKSLGRNW |
| | | ICEGTLERPQRSRGR |
| 40 | ZNF816 | MLREEATKKSKEKEPGMALPQGRLTFRDVAIEFSLEEWKCLN |
| | | PAQRALYRAVMLENYRNLEFVDSSLKSMMEFSSTRHSITGEVI |
| | | HTGTLQRHKSHHIGDFCFPEMKKDIHHFEFQWQ |
| 41 | ZNF680 | MPGPPGSLEMGPLTFRDVAIEFSLEEWQCLDTAQRNLYRKVM |
| | | FENYRNLVFLGIAVSKPHLITCLEQGKEPWNRKRQEMVAKPP |
| | | VIYSHFTEDLWPEHSIKDSF |
| 42 | ZNF41 | MSPPWSPALAAEGRGSSCEASVSFEDVTVDFSKEEWQHLDPA |
| | | QRRLYWDVTLENYSHLLSVGYQIPKSEAAFKLEQGEGPWML |
| | | EGEAPHQSCSGEAIGKMQQQGIPGGIFFHC |
| 43 | ZNF189 | MASPSPPPESKEEWDYLDPAQRSLYKDVMMENYGNLVSLDV |
| | | LNRDKDEEPTVKQEIEEIEEEVEPQGVIVTRIKSEIDQDPMGRE |
| | | TFELVGRLDKQRGIFLWEIPRESL |
| 44 | ZNF528 | MALTQGPLKFMDVAIEFSQEEWKCLDPAQRTLYRDVMLENY |
| | | RNLVSLGICLPDLSVTSMLEQKRDPWTLQSEEKIANDPDGREC |
| | | IKGVNTERSSKLGSN |
| 45 | ZNF543 | MAASAQVSVTFEDVAVTFTQEEWGQLDAAQRTLYQEVMLET |
| | | CGLLMSLGCPLFKPELIYQLDHRQELWMATKDLSQSSYPGDN |
| | | TKPKTTEPTFSHLALPE |
| 46 | ZNF554 | MFSQEERMAAGYLPRWSQELVTFEDVSMDFSQEEWELLEPA |
| | | QKNLYREVMLENYRNVVSLEALKNQCTDVGIKEGPLSPAQTS |
| | | QVTSLSSWTGYLLFQPVASSHLEQREALWIEEKGTPQASCSD |
| | | WMTVLRNQDSTYKKVALQE |
| 47 | ZNF140 | MSQGSVTFRDVAIDFSQEEWKWLQPAQRDLYRCVMLENYGH |
| | | LVSLGLSISKPDVVSLLEQGKEPWLGKREVKRDLFSVSESSGEI |
| | | KDFSPKNVIYDD |
| 48 | ZNF610 | MEEAQKRKAKESGMALPQGRLTFMDVAIEFSQEEWKSLDPG |
| | | QRALYRDVMLENYRNLVFLGRSCVLGSNAENKPIKNQLGLTL |
| | | ESHLSELQLFQAGRKIYRSNQVEKFTNHR |
| 49 | ZNF264 | MAAAVLTDRAQVSVTFDDVAVTFTKEEWGQLDLAQRTLYQE |
| | | VMLENCGLLVSLGCPVPKAELICHLEHGQEPWTRKEDLSQDT |
| | | CPGDKGKPKTTEPTTCEPALSE |
| 50 | ZNF350 | MIQAQESITLEDVAVDFTWEEWQLLGAAQKDLYRDVMLENY |
| | | SNLVAVGYQASKPDALFKLEQGEQLWTIEDGIHSGACSDIWK |
| | | VDHVLERLQSESLVNR |
| 51 | ZNF8 | MEGVAGVMSVGPPAARLQEPVTFRDVAVDFTQEEWGQLDPT |
| | | QRILYRDVMLETFGHLLSIGPELPKPEVISQLEQGTELWVAER |
| | | GTTQGCHPAWEPRSESQASRKEEGLPEE |
| 52 | ZNF582 | MSLGSELFRDVAIVFSQEEWQWLAPAQRDLYRDVMLETYSN |
| | | LVSLGLAVSKPDVISFLEQGKEPWMVERVVSGGLCPVLESRY |
| | | DTKELFPKQHVYEV |
| 53 | ZNF30 | MAHKYVGLQYHGSVTFEDVAIAFSQQEWESLDSSQRGLYRD |
| | | VMLENYRNLVSMAGHSRSKPHVIALLEQWKEPEVTVRKDGR |
| | | RWCTDLQLEDDTIGCKEMPTSEN |
| 54 | ZNF324 | MAFEDVAVYFSQEEWGLLDTAQRALYRRVMLDNFALVASLG |
| | | LSTSRPRVVIQLERGEEPWVPSGTDTTLSRTTYRRRNPGSWSL |
| | | TEDRDVSG |
| 55 | ZNF98 | MLENYRNLVFVGIAASKPDLITCLEQGKEPWNVKRHEMVTEP |
| | | PVVYSYFAQDLWPKQGKKNYFQKVILRTYKKCGRENLQLRK |
| | | YCKSMDECKVHKECYNGLNQC |
| 56 | ZNF669 | MHFRRPDPCREPLASPIQDSVAFEDVAVNFTQEEWALLDSSQ |
| | | KNLYREVMQETCRNLASVGSQWKDQNIEDHFEKPGKDIRNHI |
| | | VQRLCESKEDGQYGEVVSQIPNLDLNENISTGLKPCECSICGK |
| 57 | ZNF677 | MALSQGLFTFKDVAIEFSQEEWECLDPAQRALYRDVMLENYR |
| | | NLLSLDEDNIPPEDDISVGFTSKGLSPKENNKEELYHLVILERK |
| | | ESHGINNFDLKEVWENMPKFDSLW |
| 58 | ZNF596 | MTFEDIIVDFTQEEWALLDTSQRKLFQDVMLENISHLVSIGKQ |
| | | LCKSVVLSQLEQVEKLSTQRISLLQGREVGIKHQEIPFIHHIYQ |
| | | KGTSTISTMRS |
| 59 | ZNF214 | MAVTFEDVTIIFTWEEWKFLDSSQKRLYREVMWENYTNVMS |
| | | VENWNESYKSQEEKFRYLEYENFSYWQGWWNAGAQMYEN |
| | | QNYGETVQGTDSKDLTQQDRSQC |
| 60 | ZNF37A | MITSQGSVSFRDVTVGFTQEEWQHLDPAQRTLYRDVMLENYS |
| | | HLVSVGYCIPKPEVILKLEKGEEPWILEEKFPSQSHLELINTSRN |
| | | YSIMKFNEFNKG |
| 61 | ZNF34 | MFEDVAVYLSREEWGRLGPAQRGLYRDVMLETYGNLVSLGV |
| | | GPAGPKPGVISQLERGDEPWVLDVQGTSGKEHLRVNSPALGT |
| | | RTEYKELTSQETFGEEDPQGSEPVEACDHIS |
| 62 | ZNF250 | METYGNVVSLGLPGSKPDIISQLERGEDPWVLDRKGAKKSQG |
| | | LWSDYSDNLKYDHTTACTQQDSLSCPWECETKGESQNTDLSP |
| | | KPLISEQTVILGKTPLGRIDQENNETKQ |
| 63 | ZNF547 | MAEMNPAQGHVVFEDVAIYFSQEEWGHLDEAQRLLYRDVM |
| | | LENLALLSSLGCCHGAEDEEAPLEPGVSVGVSQVMAPKPCLS |
| | | TQNTQPCETCSSLLKDILRL |
| 64 | ZNF273 | MLDNYRNLVFLGIAVSKPDLITCLEQGKEPCNMKRHAMVAK |
| | | PPVVCSHFAQDLWPKQGLKDS |
| 65 | ZNF354A | MAAGQREARPQVSLTFEDVAVLFTRDEWRKLAPSQRNLYRD |
| | | VMLENYRNLVSLGLPFTKPKVISLLQQGEDPWEVEKDGSGVS |
| | | SLGSKSSHKTTKSTQTQDSSFQ |
| 66 | ZFP82 | MALRSVMFSDVSIDFSPEEWEYLDLEQKDLYRDVMLENYSNL |
| | | VSLGCFISKPDVISSLEQGKEPWKVVRKGRRQYPDLETKYETK |
| | | KLSLENDIYEIN |
| 67 | ZNF224 | MTTFKEAMTFKDVAVVFTEEELGLLDLAQRKLYRDVMLENF |
| | | RNLLSVGHQAFHRDTFHFLREEKIWMMKTAIQREGNSGDKIQ |
| | | TEMETVSEAGTHQEW |
| 68 | ZNF33A | MFQVEQKSQESVSFKDVTVGFTQEEWQHLDPSQRALYRDVM |
| | | LENYSNLVSVGYCVHKPEVIFRLQQGEEPWKQEEEFPSQSFPE |
| | | VWTADHLKERSQENQSKHL |
| 69 | ZNF45 | MTKSKEAVTFKDVAVVFSEEELQLLDLAQRKLYRDVMLENF |
| | | RNVVSVGHQSTPDGLPQLEREEKLWMMKMATQRDNSSGAK |
| | | NLKEMETLQEVGLRYLP |
| 70 | ZNF175 | MSQKPQVLGPEKQDGSCEASVSFEDVTVDFSREEWQQLDPAQ |
| | | RCLYRDVMLELYSHLFAVGYHIPNPEVIFRMLKEKEPRVEEAE |
| | | VSHQRCQEREFGLEIPQKEISKKASFQ |
| 71 | ZNF595 | MELVTFRDVAIEFSPEEWKCLDPAQQNLYRDVMLENYRNLVS |
| | | LGFVISNPDLVTCLEQIKEPCNLKIHETAAKPPAICSPFSQDLSP |
| | | VQGIEDSF |
| 72 | ZNF184 | MSTLLQGGHNLLSSASFQESVTFKDVIVDFTQEEWKQLDPGQ |
| | | RDLFRDVTLENYTHLVSIGLQVSKPDVISQLEQGTEPWIMEPSI |
| | | PVGTCADWETRLENSVSAPEPDISEE |
| 73 | ZNF419 | MDPAQVPVAADLLTDHEEGYVTFEDVAVYFSQEEWRLLDDA |
| | | QRLLYRNVMLENFTLLASLGLASSKTHEITQLESWEEPFMPA |
| | | WEVVTSAIPRGCWHGAEAEEAPEQIASVG |
| 74 | ZFP28-1 | MKKLEAVGTGIEPKAMSQGLVTFGDVAVDFSQEEWEWLNPI |
| | | QRNLYRKVMLENYRNLASLGLCVSKPDVISSLEQGKEPWTVK |
| | | RKMTRAWCPDLKAVWKIKELPLKKDFCEG |
| 75 | ZFP28-2 | MSLLGEHWDYDALFETQPGLVTIKNLAVDFRQQLHPAQKNF |
| | | CKNGIWENNSDLGSAGHCVAKPDLVSLLEQEKEPWMVKREL |
| | | TGSLFSGQRSVHETQELFPKQDSYAE |
| 76 | ZNF18 | MLALAASQPARLEERLIRDRDLGASLLPAAPQEQWRQLDSTQ |
| | | KEQYWDLILETYGKMVSGAGISHPKSDLTNSIEFGEELAGIYL |
| | | HVNEKIPRPTCIGDRQENDKENLNLENH |
| 77 | ZNF213 | MEGRPGETTDTCFVSGVHGPVALGDIPFYFSREEWGTLDPAQ |
| | | RDLFWDIKRENSRNTTLGFGLKGQSEKSLLQEMVPVVPGQTG |
| | | SDVTVSWSPEEAEAWESENRPRAALGPVVGARRGRPPTRRRQ |
| | | FRDLA |
| 78 | ZNF394 | MVAVVRALQRALDGTSSQGMVTFEDTAVSLTWEEWERLDPA |
| | | RRDFCRESAQKDSGSTVPPSLESRVENKELIPMQQILEEAEPQG |
| | | QLQEAFQGKRPLFSKCGSTHEDRVEKQSGDP |
| 79 | ZFP1 | MNKSQGSVSFTDVTVDFTQEEWEQLDPSQRILYMDVMLENY |
| | | SNLLSVEVWKADDQMERDHRNPDEQARQFLILKNQTPIEERG |
| | | DLFGKALNLNTDFVSLRQVPYKYDLYEKTL |
| 80 | ZFP14 | MAHGSVTFRDVAIDFSQEEWEFLDPAQRDLYRDVMWENYSN |
| | | FISLGPSISKPDVITLLDEERKEPGMVVREGTRRYCPDLESRYR |
| | | TNTLSPEKDIYEIYSFQWDIMER |
| 81 | ZNF416 | MAAAVLRDSTSVPVTAEAKLMGFTQGCVTFEDVAIYFSQEE |
| | | WGLLDEAQRLLYRDVMLENFALITALVCWHGMEDEETPEQS |
| | | VSVEGVPQVRTPEASPSTQKIQSCDMCVPFLTDILHLTDLPGQ |
| | | ELYLTGACAVFHQDQK |
| 82 | ZNF557 | MLPPTAASQREGHTEGGELVNELLKSWLKGLVTFEDVAVEFT |
| | | QEEWALLDPAQRTLYRDVMLENCRNLASLGNQVDKPRLISQL |
| | | EQEDKVMTEERGILSGTCPDVENPFKAKGLTPKLHVFRKEQS |
| | | RNMKMER |
| 83 | ZNF566 | MAQESVMFSDVSVDFSQEEWECLNDDQRDLYRDVMLENYS |
| | | NLVSMGHSISKPNVISYLEQGKEPWLADRELTRGQWPVLESR |
| | | CETKKLFLKKEIYEIESTQWEIMEK |
| 84 | ZNF729 | MPGAPGSLEMGPLTFRDVTIEFSLEEWQCLDTVQQNLYRDVM |
| | | LENYRNLVFLGMAVFKPDLITCLKQGKEPWNMKRHEMVTKP |
| | | PVMRSHFTQDLWPDQSTKDSFQEVILRTYAR |
| 85 | ZIM2 | MAGSQFPDFKHLGTFLVFEELVTFEDVLVDFSPEELSSLSAAQ |
| | | RNLYREVMLENYRNLVSLGHQFSKPDIISRLEEEESYAMETDS |
| | | RHTVICQGE |
| 86 | ZNF254 | MPGPPRSLEMGLLTFRDVAIEFSLEEWQHLDIAQQNLYRNVM |
| | | LENYRNLAFLGIAVSKPDLITCLEQGKEPWNMKRHE |
| 87 | ZNF764 | MAPPLAPLPPRDPNGAGPEWREPGAVSFADVAVYFCREEWG |
| | | CLRPAQRALYRDVMRETYGHLSALGIGGNKPALISWVEEEAE |
| | | LWGPAAQDPE |
| 88 | ZNF785 | MGPPLAPRPAHVPGEAGPRRTRESRPGAVSFADVAVYFSPEE |
| | | WECLRPAQRALYRDVMRETFGHLGALGFSVPKPAFISWVEGE |
| | | VEAWSPEAQDPDGESS |
| 89 | ZNF10 (KOX1) | MDAKSLTAWSRTLVTFKDVFVDFTREEWKLLDTAQQIVYRN |
| | | VMLENYKNLVSLGYQLTKPDVILRLEKGEEPWLVEREIHQET |
| | | HPDSETAFEIKSSVSSRSIFKDKQSCDIKMEGMARNDLWYLSL |
| | | EEVWKCRDQLDKYQENPERHLRQVAFTQKKVLTQERVSESG |
| | | KYGGNCLLPAQLVLREYFHKRDSHTKSLKHDLVLNGHQDSC |
| | | ASNSNECGQTFCQNIHLIQFARTHTGDKSYKCPDNDNSLTHGS |
| | | SLGISKGIHREKPYECKECGKFFSWRSNLTRHQLIHTGEKPYEC |
| | | KECGKSFSRSSHLIGHQKTHTGEEPYECKECGKSFSWFSHLVT |
| | | HQRTHTGDKLYTCNQCGKSFVHSSRLIRHQRTHTGEKPYECP |
| | | ECGKSFRQSTHLILHQRTHVRVRPYECNECGKSYSQRSHLVV |
| | | HHRIHTGLKPFECKDCGKCFSRSSHLYSHQRTHTGEKPYECHD |
| | | CGKSFSQSSALIVHQRIHTGEKPYECCQCGKAFIRKNDLIKHQ |
| | | RIHVGEETYKCNQCGIIFSQNSPFIVHQIAHTGEQFLTCNQCGT |
| | | ALVNTSNLIGYQTNHIRENAY |
| 90 | CBX5 | MGKKTKRTADSSSSEDEEEYVVEKVLDRRVVKGQVEYLLKW |
| | (chromoshadow | KGFSEEHNTWEPEKNLDCPELISEFMKKYKKMKEGENNKPRE |
| | domain) | KSESNKRKSNFSNSADDIKSKKKREQSNDIARGFERGLEPEKII |
| | | GATDSCGDLMFLMKWKDTDEADLVLAKEANVKCPQIVIAFY |
| | | EERLTWHAYPEDAENKEKETAKS |
| 91 | RYBP | MTMGDKKSPTRPKRQAKPAADEGFWDCSVCTFRNSAEAFKC |
| | (YAF2_RYBP | SICDVRKGTSTRKPRINSQLVAQQVAQQYATPPPPKKEKKEKV |
| | component of | EKQDKEKPEKDKEISPSVTKKNTNKKTKPKSDILKDPPSEANSI |
| | PRC1) | QSANATTKTSETNHTSRPRLKNVDRSTAQQLAVTVGNVTVIIT |
| | | DFKEKTRSSSTSSSTVTSSAGSEQQNQSSSGSESTDKGSSRSST |
| | | PKGDMSAVNDESF |
| 92 | YAF2 | MGDKKSPTRPKRQPKPSSDEGYWDCSVCTFRNSAEAFKCMM |
| | (YAF2_RYBP | CDVRKGTSTRKPRPVSQLVAQQVTQQFVPPTQSKKEKKDKVE |
| | component of | KEKSEKETTSKKNSHKKTRPRLKNVDRSSAQHLEVTVGDLTV |
| | PRC1) | IITDFKEKTKSPPASSAASADQHSQSGSSSDNTERGMSRSSSPR |
| | | GEASSLNGESH |
| 93 | MGA (component | MEEKQQIILANQDGGTVAGAAPTFFVILKQPGNGKTDQGILVT |
| | of PRC1.6) | NQDACALASSVSSPVKSKGKICLPADCTVGGITVTLDNNSMW |
| | | NEFYHRSTEMILTKQGRRMFPYCRYWITGLDSNLKYILVMDIS |
| | | PVDNHRYKWNGRWWEPSGKAEPHVLGRVFIHPESPSTGHYW |
| | | MHQPVSFYKLKLTNNTLDQEGHIILHSMHRYLPRLHLVPAEK |
| | | AVEVIQLNGPGVHTFTFPQTEFFAVTAYQNIQITQLKIDYNPFA |
| | | KGFRDDGLNNKPQRDGKQKNSSDQEGNNISSSSGHRVRLTEG |
| | | QGSEIQPGDLDPLSRGHETSGKGLEKTSLNIKRDFLGFMDTDS |
| | | ALSEVPQLKQEISECLIASSFEDDSRVASPLDQNGSFNVVIKEE |
| | | PLDDYDYELGECPEGVTVKQEETDEETDVYSNSDDDPILEKQ |
| | | LKRHNKVDNPEADHLSSKWLPSSPSGVAKAKMFKLDTGKMP |
| | | VVYLEPCAVTRSTVKISELPDNMLSTSRKDKSSMLAELEYLPT |
| | | YIENSNETAFCLGKESENGLRKHSPDLRVVQKYPLLKEPQWK |
| | | YPDISDSISTERILDDSKDSVGDSLSGKEDLGRKRTTMLKIATA |
| | | AKVVNANQNASPNVPGKRGRPRKLKLCKAGRPPKNTGKSLIS |
| | | TKNTPVSPGSTFPDVKPDLEDVDGVLFVSFESKEALDIHAVDG |
| | | TTEESSSLQASTTNDSGYRARISQLEKELIEDLKTLRHKQVIHP |
| | | GLQEVGLKLNSVDPTMSIDLKYLGVQLPLAPATSFPFWNLTG |
| | | TNPASPDAGFPFVSRTGKTNDFTKIKGWRGKFHSASASRNEG |
| | | GNSESSLKNRSAFCSDKLDEYLENEGKLMETSMGFSSNAPTSP |
| | | VVYQLPTKSTSYVRTLDSVLKKQSTISPSTSYSLKPHSVPPVSR |
| | | KAKSQNRQATFSGRTKSSYKSILPYPVSPKQKYSHVILGDKVT |
| | | KNSSGIISENQANNFVVPTLDENIFPKQISLRQAQQQQQQQQG |
| | | SRPPGLSKSQVKLMDLEDCALWEGKPRTYITEERADVSLTTLL |
| | | TAQASLKTKPIHTIIRKRAPPCNNDFCRLGCVCSSLALEKRQPA |
| | | HCRRPDCMFGCTCLKRKVVLVKGGSKTKHFQRKAAHRDPVF |
| | | YDTLGEEAREEEEGIREEEEQLKEKKKRKKLEYTICETEPEQP |
| | | VRHYPLWVKVEGEVDPEPVYIPTPSVIEPMKPLLLPQPEVLSPT |
| | | VKGKLLTGIKSPRSYTPKPNPVIREEDKDPVYLYFESMMTCAR |
| | | VRVYERKKEDQRQPSSSSSPSPSFQQQTSCHSSPENHNNAKEP |
| | | DSEQQPLKQLTCDLEDDSDKLQEKSWKSSCNEGESSSTSYMH |
| | | QRSPGGPTKLIEIISDCNWEEDRNKILSILSQHINSNMPQSLKVG |
| | | SFIIELASQRKSRGEKNPPVYSSRVKISMPSCQDQDDMAEKSG |
| | | SETPDGPLSPGKMEDISPVQTDALDSVRERLHGGKGLPFYAGL |
| | | SPAGKLVAYKRKPSSSTSGLIQVASNAKVAASRKPRTLLPSTS |
| | | NSKMASSSGTATNRPGKNLKAFVPAKRPIAARPSPGGVFTQFV |
| | | MSKVGALQQKIPGVSTPQTLAGTQKFSIRPSPVMVVTPVVSSE |
| | | PVQVCSPVTAAVTTTTPQVFLENTTAVTPMTAISDVETKETTY |
| | | SSGATTTGVVEVSETNTSTSVTSTQSTATVNLTKTTGITTPVAS |
| | | VAFPKSLVASPSTITLPVASTASTSLVVVTAAASSSMVTTPTSS |
| | | LGSVPIILSGINGSPPVSQRPENAAQIPVATPQVSPNTVKRAGPR |
| | | LLLIPVQQGSPTLRPVSNTQLQGHRMVLQPVRSPSGMNLFRHP |
| | | NGQIVQLLPLHQLRGSNTQPNLQPVMFRNPGSVMGIRLPAPSK |
| | | PSETPPSSTSSSAFSVMNPVIQAVGSSSAVNVITQAPSLLSSGAS |
| | | FVSQAGTLTLRISPPEPQSFASKTGSETKITYSSGGQPVGTASLI |
| | | PLQSGSFALLQLPGQKPVPSSILQHVASLQMKRESQNPDQKDE |
| | | TNSIKREQETKKVLQSEGEAVDPEANVIKQNSGAATSEETLND |
| | | SLEDRGDHLDEECLPEEGCATVKPSEHSCITGSHTDQDYKDV |
| | | NEEYGARNRKSSKEKVAVLEVRTISEKASNKTVQNLSKVQHQ |
| | | KLGDVKVEQQKGFDNPEENSSEFPVTFKEESKFELSGSKVME |
| | | QQSNLQPEAKEKECGDSLEKDRERWRKHLKGPLTRKCVGAS |
| | | QECKKEADEQLIKETKTCQENSDVFQQEQGISDLLGKSGITED |
| | | ARVLKTECDSWSRISNPSAFSIVPRRAAKSSRGNGHFQGHLLL |
| | | PGEQIQPKQEKKGGRSSADFTVLDLEEDDEDDNEKTDDSIDEI |
| | | VDVVSDYQSEEVDDVEKNNCVEYIEDDEEHVDIETVEELSEEI |
| | | NVAHLKTTAAHTQSFKQPSCTHISADEKAAERSRKAPPIPLKL |
| | | KPDYWSDKLQKEAEAFAYYRRTHTANERRRRGEMRDLFEKL |
| | | KITLGLLHSSKVSKSLILTRAFSEIQGLTDQADKLIGQKNLLTR |
| | | KRNILIRKVSSLSGKTEEVVLKKLEYIYAKQQALEAQKRKKK |
| | | MGSDEFDISPRISKQQEGSSASSVDLGQMFINNRRGKPLILSRK |
| | | KDQATENTSPLNTPHTSANLVMTPQGQLLTLKGPLFSGPVVA |
| | | VSPDLLESDLKPQVAGSAVALPENDDLFMMPRIVNVTSLATE |
| | | GGLVDMGGSKYPHEVPDSKPSDHLKDTVRNEDNSLEDKGRIS |
| | | SRGNRDGRVTLGPTQVFLANKDSGYPQIVDVSNMQKAQEFLP |
| | | KKISGDMRGIQYKWKESESRGERVKSKDSSFHKLKMKDLKDS |
| | | SIEMELRKVTSAIEEAALDSSELLTNMEDEDDTDETLTSLLNEI |
| | | AFLNQQLNDDSVGLAELPSSMDTEFPGDARRAFISKVPPGSRA |
| | | TFQVEHLGTGLKELPDVQGESDSISPLLLHLEDDDFSENEKQL |
| | | AEPASEPDVLKIVIDSEIKDSLLSNKKAIDGGKNTSGLPAEPES |
| | | VSSPPTLHMKTGLENSNSTDTLWRPMPKLAPLGLKVANPSSD |
| | | ADGQSLKVMPCLAPIAAKVGSVGHKMNLTGNDQEGRESKV |
| | | MPTLAPVVAKLGNSGASPSSAGK |
| 94 | CBX1 | MGKKQNKKKVEEVLEEEEEEYVVEKVLDRRVVKGKVEYLL |
| | (chromoshadow) | KWKGFSDEDNTWEPEENLDCPDLIAEFLQSQKTAHETDKSEG |
| | | GKRKADSDSEDKGEESKPKKKKEESEKPRGFARGLEPERIIGA |
| | | TDSSGELMFLMKWKNSDEADLVPAKEANVKCPQVVISFYEER |
| | | LTWHSYPSEDDDKKDDKN |
| 95 | SCMH1 | MLVCYSVLACEILWDLPCSIMGSPLGHFTWDKYLKETCSVPA |
| | (SAM_1/SPM) | PVHCFKQSYTPPSNEFKISMKLEAQDPRNTTSTCIATVVGLTG |
| | | ARLRLRLDGSDNKNDFWRLVDSAEIQPIGNCEKNGGMLQPPL |
| | | GFRLNASSWPMFLLKTLNGAEMAPIRIFHKEPPSPSHNFFKMG |
| | | MKLEAVDRKNPHFICPATIGEVRGSEVLVTFDGWRGAFDYW |
| | | CRFDSRDIFPVGWCSLTGDNLQPPGTKVVIPKNPYPASDVNTE |
| | | KPSIHSSTKTVLEHQPGQRGRKPGKKRGRTPKTLISHPISAPSK |
| | | TAEPLKFPKKRGPKPGSKRKPRTLLNPPPASPTTSTPEPDTSTV |
| | | PQDAATIPSSAMQAPTVCIYLNKNGSTGPHLDKKKVQQLPDH |
| | | FGPARASVVLQQAVQACIDCAYHQKTVFSFLKQGHGGEVISA |
| | | VFDREQHTLNLPAVNSITYVLRFLEKLCHNLRSDNLFGNQPFT |
| | | QTHLSLTAIEYSHSHDRYLPGETFVLGNSLARSLEPHSDSMDS |
| | | ASNPTNLVSTSQRHRPLLSSCGLPPSTASAVRRLCSRGVLKGS |
| | | NERRDMESFWKLNRSPGSDRYLESRDASRLSGRDPSSWTVED |
| | | VMQFVREADPQLGPHADLFRKHEIDGKALLLLRSDMMMKY |
| | | MGLKLGPALKLSYHIDRLKQGKF |
| 96 | MPP8 | MEQVAEGARVTAVPVSAADSTEELAEVEEGVGVVGEDNDAA |
| | (Chromodomain) | ARGAEAFGDSEEDGEDVFEVEKILDMKTEGGKVLYKVRWKG |
| | | YTSDDDTWEPEIHLEDCKEVLLEFRKKIAENKAKAVRKDIQRL |
| | | SLNNDIFEANSDSDQQSETKEDTSPKKKKKKLRQREEKSPDDL |
| | | KKKKAKAGKLKDKSKPDLESSLESLVFDLRTKKRISEAKEELK |
| | | ESKKPKKDEVKETKELKKVKKGEIRDLKTKTREDPKENRKTK |
| | | KEKFVESQVESESSVLNDSPFPEDDSEGLHSDSREEKQNTKSA |
| | | RERAGQDMGLEHGFEKPLDSAMSAEEDTDVRGRRKKKTPRK |
| | | AEDTRENRKLENKNAFLEKKTVPKKQRNQDRSKSAAELEKL |
| | | MPVSAQTPKGRRLSGEERGLWSTDSAEEDKETKRNESKEKYQ |
| | | KRHDSDKEEKGRKEPKGLKTLKEIRNAFDLFKLTPEEKNDVSE |
| | | NNRKREEIPLDFKTIDDHKTKENKQSLKERRNTRDETDTWAYI |
| | | AAEGDQEVLDSVCQADENSDGRQQILSLGMDLQLEWMKLED |
| | | FQKHLDGKDENFAATDAIPSNVLRDAVKNGDYITVKVALNSN |
| | | EEYNLDQEDSSGMTLVMLAAAGGQDDLLRLLITKGAKVNGR |
| | | QKNGTTALIHAAEKNFLTTVAILLEAGAFVNVQQSNGETALM |
| | | KACKRGNSDIVRLVIECGADCNILSKHQNSALHFAKQSNNVL |
| | | VYDLLKNHLETLSRVAEETIKDYFEARLALLEPVFPIACHRLC |
| | | EGPDFSTDFNYKPPQNIPEGSGILLFIFHANFLGKEVIARLCGPC |
| | | SVQAVVLNDKFQLPVFLDSHFVYSFSPVAGPNKLFIRLTEAPS |
| | | AKVKLLIGAYRVQLQ |
| 97 | SUMO3 (Rad60- | MSEEKPKEGVKTENDHINLKVAGQDGSVVQFKIKRHTPLSKL |
| | SLD) | MKAYCERQGLSMRQIRFRFDGQPINETDTPAQLEMEDEDTID |
| | | VFQQQTGGVPESSLAGHSF |
| 98 | HERC2 (Cyt-b5) | MPSESFCLAAQARLDSKWLKTDIQLAFTRDGLCGLWNEMVK |
| | | DGEIVYTGTESTQNGELPPRKDDSVEPSGTKKEDLNDKEKKD |
| | | EEETPAPIYRAKSILDSWVWGKQPDVNELKECLSVLVKEQQA |
| | | LAVQSATTTLSALRLKQRLVILERYFIALNRTVFQENVKVKW |
| | | KSSGISLPPVDKKSSRPAGKGVEGLARVGSRAALSFAFAFLRR |
| | | AWRSGEDADLCSELLQESLDALRALPEASLFDESTVSSVWLE |
| | | VVERATRFLRSVVTGDVHGTPATKGPGSIPLQDQHLALAILLE |
| | | LAVQRGTLSQMLSAILLLLQLWDSGAQETDNERSAQGTSAPL |
| | | LPLLQRFQSIICRKDAPHSEGDMHLLSGPLSPNESFLRYLTLPQ |
| | | DNELAIDLRQTAVVVMAHLDRLATPCMPPLCSSPTSHKGSLQ |
| | | EVIGWGLIGWKYYANVIGPIQCEGLANLGVTQIACAEKRFLIL |
| | | SRNGRVYTQAYNSDTLAPQLVQGLASRNIVKIAAHSDGHHYL |
| | | ALAATGEVYSWGCGDGGRLGHGDTVPLEEPKVISAFSGKQA |
| | | GKHVVHIACGSTYSAAITAEGELYTWGRGNYGRLGHGSSEDE |
| | | AIPMLVAGLKGLKVIDVACGSGDAQTLAVTENGQVWSWGD |
| | | GDYGKLGRGGSDGCKTPKLIEKLQDLDVVKVRCGSQFSIALT |
| | | KDGQVYSWGKGDNQRLGHGTEEHVRYPKLLEGLQGKKVID |
| | | VAAGSTHCLALTEDSEVHSWGSNDQCQHFDTLRVTKPEPAAL |
| | | PGLDTKHIVGIACGPAQSFAWSSCSEWSIGLRVPFVVDICSMTF |
| | | EQLDLLLRQVSEGMDGSADWPPPQEKECVAVATLNLLRLQL |
| | | HAAISHQVDPEFLGLGLGSILLNSLKQTVVTLASSAGVLSTVQ |
| | | SAAQAVLQSGWSVLLPTAEERARALSALLPCAVSGNEVNISP |
| | | GRRFMIDLLVGSLMADGGLESALHAAITAEIQDIEAKKEAQKE |
| | | KEIDEQEANASTFHRSRTPLDKDLINTGICESSGKQCLPLVQLI |
| | | QQLLRNIASQTVARLKDVARRISSCLDFEQHSRERSASLDLLL |
| | | RFQRLLISKLYPGESIGQTSDISSPELMGVGSLLKKYTALLCTHI |
| | | GDILPVAASIASTSWRHFAEVAYIVEGDFTGVLLPELVVSIVLL |
| | | LSKNAGLMQEAGAVPLLGGLLEHLDRFNHLAPGKERDDHEE |
| | | LAWPGIMESFFTGQNCRNNEEVTLIRKADLENHNKDGGFWTV |
| | | IDGKVYDIKDFQTQSLTGNSILAQFAGEDPVVALEAALQFEDT |
| | | RESMHAFCVGQYLEPDQEIVTIPDLGSLSSPLIDTERNLGLLLG |
| | | LHASYLAMSTPLSPVEIECAKWLQSSIFSGGLQTSQIHYSYNEE |
| | | KDEDHCSSPGGTPASKSRLCSHRRALGDHSQAFLQAIADNNIQ |
| | | DHNVKDFLCQIERYCRQCHLTTPIMFPPEHPVEEVGRLLLCCL |
| | | LKHEDLGHVALSLVHAGALGIEQVKHRTLPKSVVDVCRVVY |
| | | QAKCSLIKTHQEQGRSYKEVCAPVIERLRFLFNELRPAVCNDL |
| | | SIMSKFKLLSSLPRWRRIAQKIIRERRKKRVPKKPESTDDEEKI |
| | | GNEESDLEEACILPHSPINVDKRPIAIKSPKDKWQPLLSTVTGV |
| | | HKYKWLKQNVQGLYPQSPLLSTIAEFALKEEPVDVEKMRKCL |
| | | LKQLERAEVRLEGIDTILKLASKNFLLPSVQYAMFCGWQRLIP |
| | | EGIDIGEPLTDCLKDVDLIPPFNRMLLEVTFGKLYAWAVQNIR |
| | | NVLMDASAKFKELGIQPVPLQTITNENPSGPSLGTIPQARFLLV |
| | | MLSMLTLQHGANNLDLLLNSGMLALTQTALRLIGPSCDNVEE |
| | | DMNASAQGASATVLEETRKETAPVQLPVSGPELAAMMKIGT |
| | | RVMRGVDWKWGDQDGPPPGLGRVIGELGEDGWIRVQWDTG |
| | | STNSYRMGKEGKYDLKLAELPAAAQPSAEDSDTEDDSEAEQT |
| | | ERNIHPTAMMFTSTINLLQTLCLSAGVHAEIMQSEATKTLCGL |
| | | LRMLVESGTTDKTSSPNRLVYREQHRSWCTLGFVRSIALTPQV |
| | | CGALSSPQWITLLMKVVEGHAPFTATSLQRQILAVHLLQAVLP |
| | | SWDKTERARDMKCLVEKLFDFLGSLLTTCSSDVPLLRESTLRR |
| | | RRVRPQASLTATHSSTLAEEVVALLRTLHSLTQWNGLINKYIN |
| | | SQLRSITHSFVGRPSEGAQLEDYFPDSENPEVGGLMAVLAVIG |
| | | GIDGRLRLGGQVMHDEFGEGTVTRITPKGKITVQFSDMRTCR |
| | | VCPLNQLKPLPAVAFNVNNLPFTEPMLSVWAQLVNLAGSKLE |
| | | KHKIKKSTKQAFAGQVDLDLLRCQQLKLYILKAGRALLSHQD |
| | | KLRQILSQPAVQETGTVHTDDGAVVSPDLGDMSPEGPQPPMIL |
| | | LQQLLASATQPSPVKAIFDKQELEAAALAVCQCLAVESTHPSS |
| | | PGFEDCSSSEATTPVAVQHIRPARVKRRKQSPVPALPIVVQLM |
| | | EMGFSRRNIEFALKSLTGASGNASSLPGVEALVGWLLDHSDIQ |
| | | VTELSDADTVSDEYSDEEVVEDVDDAAYSMSTGAVVTESQT |
| | | YKKRADFLSNDDYAVYVRENIQVGMMVRCCRAYEEVCEGD |
| | | VGKVIKLDRDGLHDLNVQCDWQQKGGTYWVRYIHVELIGYP |
| | | PPSSSSHIKIGDKVRVKASVTTPKYKWGSVTHQSVGVVKAFS |
| | | ANGKDIIVDFPQQSHWTGLLSEMELVPSIHPGVTCDGCQMFPI |
| | | NGSRFKCRNCDDFDFCETCFKTKKHNTRHTFGRINEPGQSAVF |
| | | CGRSGKQLKRCHSSQPGMLLDSWSRMVKSLNVSSSVNQASR |
| | | LIDGSEPCWQSSGSQGKHWIRLEIFPDVLVHRLKMIVDPADSS |
| | | YMPSLVVVSGGNSLNNLIELKTININPSDTTVPLLNDCTEYHR |
| | | YIEIAIKQCRSSGIDCKIHGLILLGRIRAEEEDLAAVPFLASDNE |
| | | EEEDEKGNSGSLIRKKAAGLESAATIRTKVFVWGLNDKDQLG |
| | | GLKGSKIKVPSFSETLSALNVVQVAGGSKSLFAVTVEGKVYA |
| | | CGEATNGRLGLGISSGTVPIPRQITALSSYVVKKVAVHSGGRH |
| | | ATALTVDGKVFSWGEGDDGKLGHFSRMNCDKPRLIEALKTK |
| | | RIRDIACGSSHSAALTSSGELYTWGLGEYGRLGHGDNTTQLKP |
| | | KMVKVLLGHRVIQVACGSRDAQTLALTDEGLVFSWGDGDFG |
| | | KLGRGGSEGCNIPQNIERLNGQGVCQIECGAQFSLALTKSGVV |
| | | WTWGKGDYFRLGHGSDVHVRKPQVVEGLRGKKIVHVAVGA |
| | | LHCLAVTDSGQVYAWGDNDHGQQGNGTTTVNRKPTLVQGL |
| | | EGQKITRVACGSSHSVAWTTVDVATPSVHEPVLFQTARDPLG |
| | | ASYLGVPSDADSSAASNKISGASNSKPNRPSLAKILLSLDGNL |
| | | AKQQALSHILTALQIMYARDAVVGALMPAAMIAPVECPSFSS |
| | | AAPSDASAMASPMNGEECMLAVDIEDRLSPNPWQEKREIVSS |
| | | EDAVTPSAVTPSAPSASARPFIPVTDDLGAASIIAETMTKTKED |
| | | VESQNKAAGPEPQALDEFTSLLIADDTRVVVDLLKLSVCSRA |
| | | GDRGRDVLSAVLSGMGTAYPQVADMLLELCVTELEDVATDS |
| | | QSGRLSSQPVVVESSHPYTDDTSTSGTVKIPGAEGLRVEFDRQ |
| | | CSTERRHDPLTVMDGVNRIVSVRSGREWSDWSSELRIPGDEL |
| | | KWKFISDGSVNGWGWRFTVYPIMPAAGPKELLSDRCVLSCPS |
| | | MDLVTCLLDFRLNLASNRSIVPRLAASLAACAQLSALAASHR |
| | | MWALQRLRKLLTTEFGQSININRLLGENDGETRALSFTGSALA |
| | | ALVKGLPEALQRQFEYEDPIVRGGKQLLHSPFFKVLVALACD |
| | | LELDTLPCCAETHKWAWFRRYCMASRVAVALDKRTPLPRLF |
| | | LDEVAKKIRELMADSENMDVLHESHDIFKREQDEQLVQWMN |
| | | RRPDDWTLSAGGSGTIYGWGHNHRGQLGGIEGAKVKVPTPC |
| | | EALATLRPVQLIGGEQTLFAVTADGKLYATGYGAGGRLGIGG |
| | | TESVSTPTLLESIQHVFIKKVAVNSGGKHCLALSSEGEVYSWG |
| | | EAEDGKLGHGNRSPCDRPRVIESLRGIEVVDVAAGGAHSACV |
| | | TAAGDLYTWGKGRYGRLGHSDSEDQLKPKLVEALQGHRVV |
| | | DIACGSGDAQTLCLTDDDTVWSWGDGDYGKLGRGGSDGCK |
| | | VPMKIDSLTGLGVVKVECGSQFSVALTKSGAVYTWGKGDYH |
| | | RLGHGSDDHVRRPRQVQGLQGKKVIAIATGSLHCVCCTEDGE |
| | | VYTWGDNDEGQLGDGTTNAIQRPRLVAALQGKKVNRVACG |
| | | SAHTLAWSTSKPASAGKLPAQVPMEYNHLQEIPIIALRNRLLL |
| | | LHHLSELFCPCIPMFDLEGSLDETGLGPSVGFDTLRGILISQGK |
| | | EAAFRKVVQATMVRDRQHGPVVELNRIQVKRSRSKGGLAGP |
| | | DGTKSVFGQMCAKMSSFGPDSLLLPHRVWKVKFVGESVDDC |
| | | GGGYSESIAEICEELQNGLTPLLIVTPNGRDESGANRDCYLLSP |
| | | AARAPVHSSMFRFLGVLLGIAIRTGSPLSLNLAEPVWKQLAG |
| | | MSLTIADLSEVDKDFIPGLMYIRDNEATSEEFEAMSLPFTVPSA |
| | | SGQDIQLSSKHTHITLDNRAEYVRLAINYRLHEFDEQVAAVRE |
| | | GMARVVPVPLLSLFTGYELETMVCGSPDIPLHLLKSVATYKGI |
| | | EPSASLIQWFWEVMESFSNTERSLFLRFVWGRTRLPRTIADFR |
| | | GRDFVIQVLDKYNPPDHFLPESYTCFFLLKLPRYSCKQVLEEK |
| | | LKYAIHFCKSIDTDDYARIALTGEPAADDSSDDSDNEDVDSFA |
| | | SDSTQDYLTGH |
| 99 | BIN1 (SH3_9) | MAEMGSKGVTAGKIASNVQKKLTRAQEKVLQKLGKADETK |
| | | DEQFEQCVQNFNKQLTEGTRLQKDLRTYLASVKAMHEASKK |
| | | LNECLQEVYEPDWPGRDEANKIAENNDLLWMDYHQKLVDQ |
| | | ALLTMDTYLGQFPDIKSRIAKRGRKLVDYDSARHHYESLQTA |
| | | KKKDEAKIAKPVSLLEKAAPQWCQGKLQAHLVAQTNLLRNQ |
| | | AEEELIKAQKVFEEMNVDLQEELPSLWNSRVGFYVNTFQSIA |
| | | GLEENFHKEMSKLNQNLNDVLVGLEKQHGSNTFTVKAQPSD |
| | | NAPAKGNKSPSPPDGSPAATPEIRVNHEPEPAGGATPGATLPK |
| | | SPSQLRKGPPVPPPPKHTPSKEVKQEQILSLFEDTFVPEISVTTP |
| | | SQFEAPGPFSEQASLLDLDFDPLPPVTSPVKAPTPSGQSIPWDL |
| | | WEPTESPAGSLPSGEPSAAEGTFAVSWPSQTAEPGPAQPAEAS |
| | | EVAGGTQPAAGAQEPGETAASEAASSSLPAVVVETFPATVNG |
| | | TVEGGSGAGRLDLPPGFMFKVQAQHDYTATDTDELQLKAGD |
| | | VVLVIPFQNPEEQDEGWLMGVKESDWNQHKELEKCRGVFPE |
| | | NFTERVP |
| 100 | PCGF2 (RING | MHRTTRIKITELNPHLMCALCGGYFIDATTIVECLHSFCKTCIV |
| | finger protein | RYLETNKYCPMCDVQVHKTRPLLSIRSDKTLQDIVYKLVPGLF |
| | domain) | KDEMKRRRDFYAAYPLTEVPNGSNEDRGEVLEQEKGALSDD |
| | | EIVSLSIEFYEGARDRDEKKGPLENGDGDKEKTGVRFLRCPAA |
| | | MTVMHLAKFLRNKMDVPSKYKVEVLYEDEPLKEYYTLMDIA |
| | | YIYPWRRNGPLPLKYRVQPACKRLTLATVPTPSEGTNTSGASE |
| | | CESVSDKAPSPATLPATSSSLPSPATPSHGSPSSHGPPATHPTSP |
| | | TPPSTASGATTAANGGSLNCLQTPSSTSRGRKMTVNGAPVPPL |
| | | T |
| 101 | TOX (HMG box) | MDVRFYPPPAQPAAAPDAPCLGPSPCLDPYYCNKFDGENMY |
| | | MSMTEPSQDYVPASQSYPGPSLESEDFNIPPITPPSLPDHSLVHL |
| | | NEVESGYHSLCHPMNHNGLLPFHPQNMDLPEITVSNMLGQDG |
| | | TLLSNSISVMPDIRNPEGTQYSSHPQMAAMRPRGQPADIRQQP |
| | | GMMPHGQLTTINQSQLSAQLGLNMGGSNVPHNSPSPPGSKSA |
| | | TPSPSSSVHEDEGDDTSKINGGEKRPASDMGKKPKTPKKKKK |
| | | KDPNEPQKPVSAYALFFRDTQAAIKGQNPNATFGEVSKIVAS |
| | | MWDGLGEEQKQVYKKKTEAAKKEYLKQLAAYRASLVSKSY |
| | | SEPVDVKTSQPPQLINSKPSVFHGPSQAHSALYLSSHYHQQPG |
| | | MNPHLTAMHPSLPRNIAPKPNNQMPVTVSIANMAVSPPPPLQI |
| | | SPPLHQHLNMQQHQPLTMQQPLGNQLPMQVQSALHSPTMQQ |
| | | GFTLQPDYQTIINPTSTAAQVVTQAMEYVRSGCRNPPPQPVD |
| | | WNNDYCSSGGMQRDKALYLT |
| 102 | FOXA1 (HNF3A | MLGTVKMEGHETSDWNSYYADTQEAYSSVPVSNMNSGLGS |
| | C-terminal domain) | MNSMNTYMTMNTMTTSGNMTPASFNMSYANPGLGAGLSPG |
| | | AVAGMPGGSAGAMNSMTAAGVTAMGTALSPSGMGAMGAQ |
| | | QAASMNGLGPYAAAMNPCMSPMAYAPSNLGRSRAGGGGDA |
| | | KTFKRSYPHAKPPYSYISLITMAIQQAPSKMLTLSEIYQWIMDL |
| | | FPYYRQNQQRWQNSIRHSLSFNDCFVKVARSPDKPGKGSYWT |
| | | LHPDSGNMFENGCYLRRQKRFKCEKQPGAGGGGGSGSGGSG |
| | | AKGGPESRKDPSGASNPSADSPLHRGVHGKTGQLEGAPAPGP |
| | | AASPQTLDHSGATATGGASELKTPASSTAPPISSGPGALASVPA |
| | | SHPAHGLAPHESQLHLKGDPHYSFNHPFSINNLMSSSEQQHKL |
| | | DFKAYEQALQYSPYGSTLPASLPLGSASVTTRSPIEPSALEPAY |
| | | YQGVYSRPVLNTS |
| 103 | FOXA2 (HNF3B | MLGAVKMEGHEPSDWSSYYAEPEGYSSVSNMNAGLGMNGM |
| | C-terminal domain) | NTYMSMSAAAMGSGSGNMSAGSMNMSSYVGAGMSPSLAG |
| | | MSPGAGAMAGMGGSAGAAGVAGMGPHLSPSLSPLGGQAAG |
| | | AMGGLAPYANMNSMSPMYGQAGLSRARDPKTYRRSYTHAK |
| | | PPYSYISLITMAIQQSPNKMLTLSEIYQWIMDLFPFYRQNQQR |
| | | WQNSIRHSLSFNDCFLKVPRSPDKPGKGSFWTLHPDSGNMFE |
| | | NGCYLRRQKRFKCEKQLALKEAAGAAGSGKKAAAGAQASQ |
| | | AQLGEAAGPASETPAGTESPHSSASPCQEHKRGGLGELKGTPA |
| | | AALSPPEPAPSPGQQQQAAAHLLGPPHHPGLPPEAHLKPEHHY |
| | | AFNHPFSINNLMSSEQQHHHSHHHHQPHKMDLKAYEQVMHY |
| | | PGYGSPMPGSLAMGPVTNKTGLDASPLAADTSYYQGVYSRPI |
| | | MNSS |
| 104 | IRF2BP1 (IRF- | MASVQASRRQWCYLCDLPKMPWAMVWDFSEAVCRGCVNFE |
| | 2BP1_2 N-terminal | GADRIELLIDAARQLKRSHVLPEGRSPGPPALKHPATKDLAAA |
| | domain) | AAQGPQLPPPQAQPQPSGTGGGVSGQDRYDRATSSGRLPLPSP |
| | | ALEYTLGSRLANGLGREEAVAEGARRALLGSMPGLMPPGLLA |
| | | AAVSGLGSRGLTLAPGLSPARPLFGSDFEKEKQQRNADCLAE |
| | | LNEAMRGRAEEWHGRPKAVREQLLALSACAPFNVRFKKDHG |
| | | LVGRVFAFDATARPPGYEFELKLFTEYPCGSGNVYAGVLAVA |
| | | RQMFHDALREPGKALASSGFKYLEYERRHGSGEWRQLGELL |
| | | TDGVRSFREPAPAEALPQQYPEPAPAALCGPPPRAPSRNLAPT |
| | | PRRRKASPEPEGEAAGKMTTEEQQQRHWVAPGGPYSAETPG |
| | | VPSPIAALKNVAEALGHSPKDPGGGGGPVRAGGASPAASSTA |
| | | QPPTQHRLVARNGEAEVSPTAGAEAVSGGGSGTGATPGAPLC |
| | | CTLCRERLEDTHFVQCPSVPGHKFCFPCSREFIKAQGPAGEVY |
| | | CPSGDKCPLVGSSVPWAFMQGEIATILAGDIKVKKERDP |
| 105 | IRF2BP2 (IRF- | MAAAVAVAAASRRQSCYLCDLPRMPWAMIWDFTEPVCRGC |
| | 2BP1_2 N-terminal | VNYEGADRVEFVIETARQLKRAHGCFPEGRSPPGAAASAAAK |
| | domain) | PPPLSAKDILLQQQQQLGHGGPEAAPRAPQALERYPLAAAAE |
| | | RPPRLGSDFGSSRPAASLAQPPTPQPPPVNGILVPNGFSKLEEPP |
| | | ELNRQSPNPRRGHAVPPTLVPLMNGSATPLPTALGLGGRAAA |
| | | SLAAVSGTAAASLGSAQPTDLGAHKRPASVSSSAAVEHEQRE |
| | | AAAKEKQPPPPAHRGPADSLSTAAGAAELSAEGAGKSRGSGE |
| | | QDWVNRPKTVRDTLLALHQHGHSGPFESKFKKEPALTAGRLL |
| | | GFEANGANGSKAVARTARKRKPSPEPEGEVGPPKINGEAQPW |
| | | LSTSTEGLKIPMTPTSSFVSPPPPTASPHSNRTTPPEAAQNGQSP |
| | | MAALILVADNAGGSHASKDANQVHSTTRRNSNSPPSPSSMNQ |
| | | RRLGPREVGGQGAGNTGGLEPVHPASLPDSSLATSAPLCCTLC |
| | | HERLEDTHFVQCPSVPSHKFCFPCSRQSIKQQGASGEVYCPSG |
| | | EKCPLVGSNVPWAFMQGEIATILAGDVKVKKERDS |
| 106 | IRF2BPL IRF- | MSAAQVSSSRRQSCYLCDLPRMPWAMIWDFSEPVCRGCVNY |
| | 2BP1_2 N-terminal | EGADRIEFVIETARQLKRAHGCFQDGRSPGPPPPVGVKTVALS |
| | domain | AKEAAAAAAAAAAAAAAAQQQQQQQQQQQQQQQQQQQQQ |
| | | QQQQLNHVDGSSKPAVLAAPSGLERYGLSAAAAAAAAAAAA |
| | | VEQRSRFEYPPPPVSLGSSSHTARLPNGLGGPNGFPKPTPEEGP |
| | | PELNRQSPNSSSAAASVASRRGTHGGLVTGLPNPGGGGGPQL |
| | | TVPPNLLPQTLLNGPASAAVLPPPPPHALGSRGPPTPAPPGAPG |
| | | GPACLGGTPGVSATSSSASSSTSSSVAEVGVGAGGKRPGSVSS |
| | | TDQERELKEKQRNAEALAELSESLRNRAEEWASKPKMVRDT |
| | | LLTLAGCTPYEVRFKKDHSLLGRVFAFDAVSKPGMDYELKLF |
| | | IEYPTGSGNVYSSASGVAKQMYQDCMKDFGRGLSSGFKYLE |
| | | YEKKHGSGDWRLLGDLLPEAVRFFKEGVPGADMLPQPYLDA |
| | | SCPMLPTALVSLSRAPSAPPGTGALPPAAPSGRGAAASLRKRK |
| | | ASPEPPDSAEGALKLGEEQQRQQWMANQSEALKLTMSAGGF |
| | | AAPGHAAGGPPPPPPPLGPHSNRTTPPESAPQNGPSPMAALMS |
| | | VADTLGTAHSPKDGSSVHSTTASARRNSSSPVSPASVPGQRRL |
| | | ASRNGDLNLQVAPPPPSAHPGMDQVHPQNIPDSPMANSGPLC |
| | | CTICHERLEDTHFVQCPSVPSHKFCFPCSRESIKAQGATGEVYC |
| | | PSGEKCPLVGSNVPWAFMQGEIATILAGDVKVKKERDP |
| 107 | HOXA13 | MTASVLLHPRWIEPTVMFLYDNGGGLVADELNKNMEGAAA |
| | (homeodomain) | AAAAAAAAAAAGAGGGGFPHPAAAAAGGNFSVAAAAAAAA |
| | | AAAANQCRNLMAHPAPLAPGAASAYSSAPGEAPPSAAAAAA |
| | | AAAAAAAAAAAASSSGGPGPAGPAGAEAAKQCSPCSAAAQS |
| | | SSGPAALPYGYFGSGYYPCARMGPHPNAIKSCAQPASAAAAA |
| | | AFADKYMDTAGPAAEEFSSRAKEFAFYHQGYAAGPYHHHQP |
| | | MPGYLDMPVVPGLGGPGESRHEPLGLPMESYQPWALPNGWN |
| | | GQMYCPKEQAQPPHLWKSTLPDVVSHPSDASSYRRGRKKRV |
| | | PYTKVQLKELEREYATNKFITKDKRRRISATTNLSERQVTIWF |
| | | QNRRVKEKKVINKLKTTS |
| 108 | HOXB13 | MEPGNYATLDGAKDIEGLLGAGGGRNLVAHSPLTSHPAAPTL |
| | (homeodomain) | MPAVNYAPLDLPGSAEPPKQCHPCPGVPQGTSPAPVPYGYFG |
| | | GGYYSCRVSRSSLKPCAQAATLAAYPAETPTAGEEYPSRPTEF |
| | | AFYPGYPGTYQPMASYLDVSVVQTLGAPGEPRHDSLLPVDSY |
| | | QSWALAGGWNSQMCCQGEQNPPGPFWKAAFADSSGQHPPD |
| | | ACAFRRGRKKRIPYSKGQLRELEREYAANKFITKDKRRKISAA |
| | | TSLSERQITIWFQNRRVKEKKVLAKVKNSATP |
| 109 | HOXC13 | MTTSLLLHPRWPESLMYVYEDSAAESGIGGGGGGGGGGTGG |
| | (homeodomain) | AGGGCSGASPGKAPSMDGLGSSCPASHCRDLLPHPVLGRPPA |
| | | PLGAPQGAVYTDIPAPEAARQCAPPPAPPTSSSATLGYGYPFG |
| | | GSYYGCRLSHNVNLQQKPCAYHPGDKYPEPSGALPGDDLSSR |
| | | AKEFAFYPSFASSYQAMPGYLDVSVVPGISGHPEPRHDALIPV |
| | | EGYQHWALSNGWDSQVYCSKEQSQSAHLWKSPFPDVVPLQP |
| | | EVSSYRRGRKKRVPYTKVQLKELEKEYAASKFITKEKRRRISA |
| | | TTNLSERQVTIWFQNRRVKEKKVVSKSKAPHLHST |
| 110 | HOXA11 | MDFDERGPCSSNMYLPSCTYYVSGPDFSSLPSFLPQTPSSRPM |
| | (homeodomain) | TYSYSSNLPQVQPVREVTFREYAIEPATKWHPRGNLAHCYSA |
| | | EELVHRDCLQAPSAAGVPGDVLAKSSANVYHHPTPAVSSNFY |
| | | STVGRNGVLPQAFDQFFETAYGTPENLASSDYPGDKSAEKGP |
| | | PAATATSAAAAAAATGAPATSSSDSGGGGGCRETAAAAEEKE |
| | | RRRRPESSSSPESSSGHTEDKAGGSSGQRTRKKRCPYTKYQIR |
| | | ELEREFFFSVYINKEKRLQLSRMLNLTDRQVKIWFQNRRMKE |
| | | KKINRDRLQYYSANPLL |
| 111 | HOXC11 | MFNSVNLGNFCSPSRKERGADFGERGSCASNLYLPSCTYYMP |
| | (homeodomain) | EFSTVSSFLPQAPSRQISYPYSAQVPPVREVSYGLEPSGKWHH |
| | | RNSYSSCYAAADELMHRECLPPSTVTEILMKNEGSYGGHHHP |
| | | SAPHATPAGFYSSVNKNSVLPQAFDRFFDNAYCGGGDPPAEP |
| | | PCSGKGEAKGEPEAPPASGLASRAEAGAEAEAEEENTNPSSSG |
| | | SAHSVAKEPAKGAAPNAPRTRKKRCPYSKFQIRELEREFFFNV |
| | | YINKEKRLQLSRMLNLTDRQVKIWFQNRRMKEKKLSRDRLQ |
| | | YFSGNPLL |
| 112 | HOXC10 | MTCPRNVTPNSYAEPLAAPGGGERYSRSAGMYMQSGSDFNC |
| | (homeodomain) | GVMRGCGLAPSLSKRDEGSSPSLALNTYPSYLSQLDSWGDPK |
| | | AAYRLEQPVGRPLSSCSYPPSVKEENVCCMYSAEKRAKSGPE |
| | | AALYSHPLPESCLGEHEVPVPSYYRASPSYSALDKTPHCSGAN |
| | | DFEAPFEQRASLNPRAEHLESPQLGGKVSFPETPKSDSQTPSPN |
| | | EIKTEQSLAGPKGSPSESEKERAKAADSSPDTSDNEAKEEIKAE |
| | | NTTGNWLTAKSGRKKRCPYTKHQTLELEKEFLFNMYLTRERR |
| | | LEISKTINLTDRQVKIWFQNRRMKLKKMNRENRIRELTSNFNF |
| | | T |
| 113 | HOXA10 | MSARKGYLLPSPNYPTTMSCSESPAANSFLVDSLISSGRGEAG |
| | (homeodomain) | GGGGGAGGGGGGGYYAHGGVYLPPAADLPYGLQSCGLFPTL |
| | | GGKRNEAASPGSGGGGGGLGPGAHGYGPSPIDLWLDAPRSCR |
| | | MEPPDGPPPPPQQQPPPPPQPPQPAPQATSCSFAQNIKEESSYC |
| | | LYDSADKCPKVSATAAELAPFPRGPPPDGCALGTSSGVPVPGY |
| | | FRLSQAYGTAKGYGSGGGGAQQLGAGPFPAQPPGRGFDLPPA |
| | | LASGSADAARKERALDSPPPPTLACGSGGGSQGDEEAHASSS |
| | | AAEELSPAPSESSKASPEKDSLGNSKGENAANWLTAKSGRKK |
| | | RCPYTKHQTLELEKEFLFNMYLTRERRLEISRSVHLTDRQVKI |
| | | WFQNRRMKLKKMNRENRIRELTANFNFS |
| 114 | HOXB9 | MSISGTLSSYYVDSIISHESEDAPPAKFPSGQYASSRQPGHAEH |
| | (homeodomain) | LEFPSCSFQPKAPVFGASWAPLSPHASGSLPSVYHPYIQPQGVP |
| | | PAESRYLRTWLEPAPRGEAAPGQGQAAVKAEPLLGAPGELLK |
| | | QGTPEYSLETSAGREAVLSNQRPGYGDNKICEGSEDKERPDQT |
| | | NPSANWLHARSSRKKRCPYTKYQTLELEKEFLFNMYLTRDRR |
| | | HEVARLLNLSERQVKIWFQNRRMKMKKMNKEQGKE |
| 115 | HOXA9 | MATTGALGNYYVDSFLLGADAADELSVGRYAPGTLGQPPRQ |
| | (homeodomain) | AATLAEHPDFSPCSFQSKATVEGASWNPVHAAGANAVPAAV |
| | | YHHHHHHPYVHPQAPVAAAAPDGRYMRSWLEPTPGALSFAG |
| | | LPSSRPYGIKPEPLSARRGDCPTLDTHTLSLTDYACGSPPVDRE |
| | | KQPSEGAFSENNAENESGGDKPPIDPNNPAANWLHARSTRKK |
| | | RCPYTKHQTLELEKEFLFNMYLTRDRRYEVARLLNLTERQVK |
| | | IWFQNRRMKMKKINKDRAKDE |
| 116 | ZFP28_HUMAN | NKKLEAVGTGIEPKAMSQGLVTFGDVAVDFSQEEWEWLNPIQ |
| | | RNLYRKVMLENYRNLASLGLCVSKPDVISSLEQGKEPW |
| 117 | ZN334_HUMAN | KMKKFQIPVSFQDLTVNFTQEEWQQLDPAQRLLYRDVMLEN |
| | | YSNLVSVGYHVSKPDVIFKLEQGEEPWIVEEFSNQNYPD |
| 118 | ZN568_HUMAN | CSQESALSEEEEDTTRPLETVTFKDVAVDLTQEEWEQMKPAQ |
| | | RNLYRDVMLENYSNLVTVGCQVTKPDVIFKLEQEEEPW |
| 119 | ZN37A_HUMAN | ITSQGSVSFRDVTVGFTQEEWQHLDPAQRTLYRDVMLENYSH |
| | | LVSVGYCIPKPEVILKLEKGEEPWILEEKFPSQSHLEL |
| 120 | ZN181_HUMAN | PQVTFNDVAIDFTHEEWGWLSSAQRDLYKDVMVQNYENLVS |
| | | VAGLSVTKPYVITLLEDGKEPWMMEKKLSKGMIPDWESR |
| 121 | ZN510_HUMAN | PLRFSTLFQEQQKMNISQASVSFKDVTIEFTQEEWQQMAPVQ |
| | | KNLYRDVMLENYSNLVSVGYCCFKPEVIFKLEQGEEPW |
| 122 | ZN862_HUMAN | QDPSAEGLSEEVPVVFEELPVVFEDVAVYFTREEWGMLDKRQ |
| | | KELYRDVMRMNYELLASLGPAAAKPDLISKLERRAAPW |
| 123 | ZN140_HUMAN | SQGSVTFRDVAIDFSQEEWKWLQPAQRDLYRCVMLENYGHL |
| | | VSLGLSISKPDVVSLLEQGKEPWLGKREVKRDLFSVSES |
| 124 | ZN208_HUMAN | GSLTFRDVAIEFSLEEWQCLDTAQQNLYRNVMLENYRNLVFL |
| | | GIAAFKPDLIIFLEEGKESWNMKRHEMVEESPVICSHF |
| 125 | ZN248_HUMAN | NKSQEQVSFKDVCVDFTQEEWYLLDPAQKILYRDVILENYSN |
| | | LVSVGYCITKPEVIFKIEQGEEPWILEKGFPSQCHPER |
| 126 | ZN571_HUMAN | PHLLVTFRDVAIDFSQEEWECLDPAQRDLYRDVMLENYSNLIS |
| | | LDLESSCVTKKLSPEKEIYEMESLQWENMGKRINHHL |
| 127 | ZN699_HUMAN | EEERKTAELQKNRIQDSVVFEDVAVDFTQEEWALLDLAQRNL |
| | | YRDVMLENFQNLASLGYPLHTPHLISQWEQEEDLQTVK |
| 128 | ZN726_HUMAN | GLLTFRDVAIEFSLEEWQCLDTAQKNLYRNVMLENYRNLAFL |
| | | GIAVSKPDLIICLEKEKEPWNMKRDEMVDEPPGICPHF |
| 129 | ZIK1_HUMAN | RAPTQVTVSPETHMDLTKGCVTFEDIAIYFSQDEWGLLDEAQ |
| | | RLLYLEVMLENFALVASLGCGHGTEDEETPSDQNVSVG |
| 130 | ZNF2_HUMAN | AAVSPTTRCQESVTFEDVAVVFTDEEWSRLVPIQRDLYKEVM |
| | | LENYNSIVSLGLPVPQPDVIFQLKRGDKPWMVDLHGSE |
| 131 | Z705F_HUMAN | HSLEKVTFEDVAIDFTQEEWDMMDTSKRKLYRDVMLENISHL |
| | | VSLGYQISKSYIILQLEQGKELWREGRVFLQDQNPDRE |
| 132 | ZNF14_HUMAN | DSVSFEDVAVNFTLEEWALLDSSQKKLYEDVMQETFKNLVCL |
| | | GKKWEDQDIEDDHRNQGKNRRCHMVERLCESRRGSKCG |
| 133 | ZN471_HUMAN | NVEVVKVMPQDLVTFKDVAIDFSQEEWQWMNPAQKRLYRS |
| | | MMLENYQSLVSLGLCISKPYVISLLEQGREPWEMTSEMTR |
| 134 | ZN624_HUMAN | TQPDEDLHLQAEETQLVKESVTFKDVAIDFTLEEWRLMDPTQ |
| | | RNLHKDVMLENYRNLVSLGLAVSKPDMISHLENGKGPW |
| 135 | ZNF84_HUMAN | TMLQESFSFDDLSVDFTQKEWQLLDPSQKNLYKDVMLENYSS |
| | | LVSLGYEVMKPDVIFKLEQGEEPWVGDGEIPSSDSPEV |
| 136 | ZNF7_HUMAN | EVVTFGDVAVHFSREEWQCLDPGQRALYREVMLENHSSVAG |
| | | LAGFLVFKPELISRLEQGEEPWVLDLQGAEGTEAPRTSK |
| 137 | ZN891_HUMAN | RNAEEERMIAVFLTTWLQEPMTFKDVAVEFTQEEWMMLDSA |
| | | QRSLYRDVMLENYRNLTSVEYQLYRLTVISPLDQEEIRN |
| 138 | ZN337_HUMAN | GPQGARRQAFLAFGDVTVDFTQKEWRLLSPAQRALYREVTLE |
| | | NYSHLVSLGILHSKPELIRRLEQGEVPWGEERRRRPGP |
| 139 | Z705G_HUMAN | HSLKKLTFEDVAIDFTQEEWAMMDTSKRKLYRDVMLENISHL |
| | | VSLGYQISKSYIILQLEQGKELWREGRVFLQDQNPNRE |
| 140 | ZN529_HUMAN | MPEVEFPDQFFTVLTMDHELVTLRDVVINFSQEEWEYLDSAQ |
| | | RNLYWDVMMENYSNLLSLDLESRNETKHLSVGKDIIQN |
| 141 | ZN729_HUMAN | PGAPGSLEMGPLTFRDVTIEFSLEEWQCLDTVQQNLYRDVML |
| | | ENYRNLVFLGMAVFKPDLITCLKQGKEPWNMKRHEMVT |
| 142 | ZN419_HUMAN | RDPAQVPVAADLLTDHEEGYVTFEDVAVYFSQEEWRLLDDA |
| | | QRLLYRNVMLENFTLLASLGLASSKTHEITQLESWEEPF |
| 143 | Z705A_HUMAN | HSLKKVTFEDVAIDFTQEEWAMMDTSKRKLYRDVMLENISH |
| | | LVSLGYQISKSYIILQLEQGKELWREGREFLQDQNPDRE |
| 144 | ZNF45_HUMAN | TKSKEAVTFKDVAVVFSEEELQLLDLAQRKLYRDVMLENFRN |
| | | VVSVGHQSTPDGLPQLEREEKLWMMKMATQRDNSSGAK |
| 145 | ZN302_HUMAN | SQVTFSDVAIDFSHEEWACLDSAQRDLYKDVMVQNYENLVS |
| | | VGLSVTKPYVIMLLEDGKEPWMMEKKLSKAYPFPLSHSV |
| 146 | ZN486_HUMAN | PGPLRSLEMESLQFRDVAVEFSLEEWHCLDTAQQNLYRDVML |
| | | ENYRHLVFLGIIVSKPDLITCLEQGIKPLTMKRHEMIA |
| 147 | ZN621_HUMAN | LQTTWPQESVTFEDVAVYFTQNQWASLDPAQRALYGEVMLE |
| | | NYANVASLVAFPFPKPALISHLERGEAPWGPDPWDTEIL |
| 148 | ZN688_HUMAN | APLLAPRPGETRPGCRKPGTVSFADVAVYFSPEEWGCLRPAQ |
| | | RALYRDVMQETYGHLGALGFPGPKPALISWMEQESEAW |
| 149 | ZN33A_HUMAN | NKVEQKSQESVSFKDVTVGFTQEEWQHLDPSQRALYRDVML |
| | | ENYSNLVSVGYCVHKPEVIFRLQQGEEPWKQEEEFPSQS |
| 150 | ZN554_HUMAN | CFSQEERMAAGYLPRWSQELVTFEDVSMDFSQEEWELLEPAQ |
| | | KNLYREVMLENYRNVVSLEALKNQCTDVGIKEGPLSPA |
| 151 | ZN878_HUMAN | DSVAFEDVAVNFTQEEWALLDPSQKNLYREVMQETLRNLTSI |
| | | GKKWNNQYIEDEHQNPRRNLRRLIGERLSESKESHQHG |
| 152 | ZN772_HUMAN | MGPAQVPMNSEVIVDPIQGQVNFEDVFVYFSQEEWVLLDEAQ |
| | | RLLYRDVMLENFALMASLGHTSFMSHIVASLVMGSEPW |
| 153 | ZN224_HUMAN | TTFKEAMTFKDVAVVFTEEELGLLDLAQRKLYRDVMLENFR |
| | | NLLSVGHQAFHRDTFHFLREEKIWMMKTAIQREGNSGDK |
| 154 | ZN184_HUMAN | DSTLLQGGHNLLSSASFQEAVTFKDVIVDFTQEEWKQLDPGQ |
| | | RDLFRDVTLENYTHLVSIGLQVSKPDVISQLEQGTEPW |
| 155 | ZN544_HUMAN | EARSMLVPPQASVCFEDVAMAFTQEEWEQLDLAQRTLYREV |
| | | TLETWEHIVSLGLFLSKSDVISQLEQEEDLCRAEQEAPR |
| 156 | ZNF57_HUMAN | DSVVFEDVAVDFTLEEWALLDSAQRDLYRDVMLETFRNLAS |
| | | VDDGTQFKANGSVSLQDMYGQEKSKEQTIPNFTGNNSCA |
| 157 | ZN283_HUMAN | EESHGALISSCNSRTMTDGLVTFRDVAIDFSQEEWECLDPAQR |
| | | DLYVDVMLENYSNLVSLDLESKTYETKKIFSENDIFE |
| 158 | ZN549_HUMAN | VITPQIPMVTEEFVKPSQGHVTFEDIAVYFSQEEWGLLDEAQR |
| | | CLYHDVMLENFSLMASVGCLHGIEAEEAPSEQTLSAQ |
| 159 | ZN211_HUMAN | VQLRPQTRMATALRDPASGSVTFEDVAVYFSWEEWDLLDEA |
| | | QKHLYFDVMLENFALTSSLGCWCGVEHEETPSEQRISGE |
| 160 | ZN615_HUMAN | MQAQESLTLEDVAVDFTWEEWQFLSPAQKDLYRDVMLENYS |
| | | NLVAVGYQASKPDALSKLERGEETCTTEDEIYSRICSEI |
| 161 | ZN253_HUMAN | GPLQFRDVAIEFSLEEWHCLDTAQRNLYRDVMLENYRNLVFL |
| | | GIVVSKPDLVTCLEQGKKPLTMERHEMIAKPPVMSSHF |
| 162 | ZN226_HUMAN | NMFKEAVTFKDVAVAFTEEELGLLGPAQRKLYRDVMVENFR |
| | | NLLSVGHPPFKQDVSPIERNEQLWIMTTATRRQGNLGEK |
| 163 | ZN730_HUMAN | GALTFRDVAIEFSLEEWQCLDTEQQNLYRNVMLDNYRNLVFL |
| | | GIAVSKPDLITCLEQEKEPWNLKTHDMVAKPPVICSHI |
| 164 | Z585A_HUMAN | SPQKSSALAPEDHGSSYEGSVSFRDVAIDFSREEWRHLDPSQR |
| | | NLYRDVMLETYSHLLSVGYQVPEAEVVMLEQGKEPWA |
| 165 | ZN732_HUMAN | ELLTFRDVAIEFSPEEWKCLDPAQQNLYRDVMLENYRNLISLG |
| | | VAISNPDLVIYLEQRKEPYKVKIHETVAKHPAVCSHF |
| 166 | ZN681_HUMAN | EPLKFRDVAIEFSLEEWQCLDTIQQNLYRNVMLENYRNLVFL |
| | | GIVVSKPDLITCLEQEKEPWTRKRHRMVAEPPVICSHF |
| 167 | ZN667_HUMAN | PSARGKSKSKAPITFGDLAIYFSQEEWEWLSPIQKDLYEDVML |
| | | ENYRNLVSLGLSFRRPNVITLLEKGKAPWMVEPVRRR |
| 168 | ZN649_HUMAN | TKAQESLTLEDVAVDFTWEEWQFLSPAQKDLYRDVMLENYS |
| | | NLVSVGYQAGKPDALTKLEQGEPLWTLEDEIHSPAHPEI |
| 169 | ZN470_HUMAN | SQEEVEVAGIKLCKAMSLGSVTFTDVAIDFSQDEWEWLNLAQ |
| | | RSLYKKVMLENYRNLVSVGLCISKPDVISLLEQEKDPW |
| 170 | ZN484_HUMAN | TKSLESVSFKDVTVDFSRDEWQQLDLAQKSLYREVMLENYFN |
| | | LISVGCQVPKPEVIFSLEQEEPCMLDGEIPSQSRPDGD |
| 171 | ZN431_HUMAN | SGCPGAERNLLVYSYFEKETLTFRDVAIEFSLEEWECLNPAQQ |
| | | NLYMNVMLENYKNLVFLGVAVSKQDPVTCLEQEKEPW |
| 172 | ZN382_HUMAN | PLQGSVSFKDVTVDFTQEEWQQLDPAQKALYRDVMLENYCH |
| | | FVSVGFHMAKPDMIRKLEQGEELWTQRIFPSYSYLEEDG |
| 173 | ZN254_HUMAN | PGPPRSLEMGLLTFRDVAIEFSLEEWQHLDIAQQNLYRNVMLE |
| | | NYRNLAFLGIAVSKPDLITCLEQGKEPWNMKRHEMVD |
| 174 | ZN124_HUMAN | SGHPGSWEMNSVAFEDVAVNFTQEEWALLDPSQKNLYRDV |
| | | MQETFRNLASIGNKGEDQSIEDQYKNSSRNLRHIISHSGN |
| 175 | ZN607_HUMAN | SYGSITFGDVAIDFSHQEWEYLSLVQKTLYQEVMMENYDNLV |
| | | SLAGHSVSKPDLITLLEQGKEPWMIVREETRGECTDLD |
| 176 | ZN317_HUMAN | DLFVCSGLEPHTPSVGSQESVTFQDVAVDFTEKEWPLLDSSQR |
| | | KLYKDVMLENYSNLTSLGYQVGKPSLISHLEQEEEPR |
| 177 | ZN620_HUMAN | FQTAWRQEPVTFEDVAVYFTQNEWASLDSVQRALYREVMLE |
| | | NYANVASLAFPFTTPVLVSQLEQGELPWGLDPWEPMGRE |
| 178 | ZN141_HUMAN | ELLTFRDVAIEFSPEEWKCLDPDQQNLYRDVMLENYRNLVSL |
| | | GVAISNPDLVTCLEQRKEPYNVKIHKIVARPPAMCSHF |
| 179 | ZN584_HUMAN | AGEAEAQLDPSLQGLVMFEDVTVYFSREEWGLLNVTQKGLY |
| | | RDVMLENFALVSSLGLAPSRSPVFTQLEDDEQSWVPSWV |
| 180 | ZN540_HUMAN | AHALVTFRDVAIDFSQKEWECLDTTQRKLYRDVMLENYNNL |
| | | VSLGYSGSKPDVITLLEQGKEPCVVARDVTGRQCPGLLS |
| 181 | ZN75D_HUMAN | KRIKHWKMASKLILPESLSLLTFEDVAVYFSEEEWQLLNPLEK |
| | | TLYNDVMQDIYETVISLGLKLKNDTGNDHPISVSTSE |
| 182 | ZN555_HUMAN | DSVVFEDVAVDFTLEEWALLDSAQRDLYRDVMLETFQNLAS |
| | | VDDETQFKASGSVSQQDIYGEKIPKESKIATFTRNVSWA |
| 183 | ZN658_HUMAN | NMSQASVSFQDVTVEFTREEWQHLGPVERTLYRDVMLENYS |
| | | HLISVGYCITKPKVISKLEKGEEPWSLEDEFLNQRYPGY |
| 184 | ZN684_HUMAN | ISFQESVTFQDVAVDFTAEEWQLLDCAERTLYWDVMLENYR |
| | | NLISVGCPITKTKVILKVEQGQEPWMVEGANPHESSPES |
| 185 | RBAK_HUMAN | NTLQGPVSFKDVAVDFTQEEWQQLDPDEKITYRDVMLENYS |
| | | HLVSVGYDTTKPNVIIKLEQGEEPWIMGGEFPCQHSPEA |
| 186 | ZN829_HUMAN | HPEEEERMHDELLQAVSKGPVMFRDVSIDFSQEEWECLDADQ |
| | | MNLYKEVMLENFSNLVSVGLSNSKPAVISLLEQGKEPW |
| 187 | ZN582_HUMAN | SLGSELFRDVAIVFSQEEWQWLAPAQRDLYRDVMLETYSNLV |
| | | SLGLAVSKPDVISFLEQGKEPWMVERVVSGGLCPVLES |
| 188 | ZN112_HUMAN | TKFQEMVTFKDVAVVFTEEELGLLDSVQRKLYRDVMLENFR |
| | | NLLLVAHQPFKPDLISQLEREEKLLMVETETPRDGCSGR |
| 189 | ZN716_HUMAN | AKRPGPPGSREMGLLTFRDIAIEFSLAEWQCLDHAQQNLYRD |
| | | VMLENYRNLVSLGIAVSKPDLITCLEQNKEPQNIKRNE |
| 190 | HKR1_HUMAN | TCMVHRQTMSCSGAGGITAFVAFRDVAVYFTQEEWRLLSPA |
| | | QRTLHREVMLETYNHLVSLEIPSSKPKLIAQLERGEAPW |
| 191 | ZN350_HUMAN | IQAQESITLEDVAVDFTWEEWQLLGAAQKDLYRDVMLENYS |
| | | NLVAVGYQASKPDALFKLEQGEQLWTIEDGIHSGACSDI |
| 192 | ZN480_HUMAN | AQKRRKRKAKESGMALPQGHLTFRDVAIEFSQAEWKCLDPA |
| | | QRALYKDVMLENYRNLVSLGISLPDLNINSMLEQRREPW |
| 193 | ZN416_HUMAN | DSTSVPVTAEAKLMGFTQGCVTFEDVAIYFSQEEWGLLDEAQ |
| | | RLLYRDVMLENFALITALVCWHGMEDEETPEQSVSVEG |
| 194 | ZNF92_HUMAN | GPLTFRDVKIEFSLEEWQCLDTAQRNLYRDVMLENYRNLVFL |
| | | GIAVSKPDLITWLEQGKEPWNLKRHEMVDKTPVMCSHF |
| 195 | ZN100_HUMAN | SGCPGAERSLLVQSYFEKGPLTFRDVAIEFSLEEWQCLDSAQQ |
| | | GLYRKVMLENYRNLVFLAGIALTKPDLITCLEQGKEP |
| 196 | ZN736_HUMAN | GVLTFRDVAVEFSPEEWECLDSAQQRLYRDVMLENYGNLVS |
| | | LGLAIFKPDLMTCLEQRKEPWKVKRQEAVAKHPAGSFHF |
| 197 | ZNF74_HUMAN | KENLEDISGWGLPEARSKESVSFKDVAVDFTQEEWGQLDSPQ |
| | | RALYRDVMLENYQNLLALGPPLHKPDVISHLERGEEPW |
| 198 | CBX1_HUMAN | EESEKPRGFARGLEPERIIGATDSSGELMFLMKWKNSDEADLV |
| | | PAKEANVKCPQVVISFYEERLTWHSYPSEDDDKKDDK |
| 199 | ZN443_HUMAN | ASVALEDVAVNFTREEWALLGPCQKNLYKDVMQETIRNLDC |
| | | VVMKWKDQNIEDQYRYPRKNLRCRMLERFVESKDGTQCG |
| 200 | ZN195_HUMAN | TLLTFRDVAIEFSLEEWKCLDLAQQNLYRDVMLENYRNLFSV |
| | | GLTVCKPGLITCLEQRKEPWNVKRQEAADGHPEMGFHH |
| 201 | ZN530_HUMAN | AAALRAPTQQVFVAFEDVAIYFSQEEWELLDEMQRLLYRDV |
| | | MLENFAVMASLGCWCGAVDEGTPSAESVSVEELSQGRTP |
| 202 | ZN782_HUMAN | NTFQASVSFQDVTVEFSQEEWQHMGPVERTLYRDVMLENYS |
| | | HLVSVGYCFTKPELIFTLEQGEDPWLLEKEKGFLSRNSP |
| 203 | ZN791_HUMAN | DSVAFEDVSVSFSQEEWALLAPSQKKLYRDVMQETFKNLASI |
| | | GEKWEDPNVEDQHKNQGRNLRSHTGERLCEGKEGSQCA |
| 204 | ZN331_HUMAN | AQGLVTFADVAIDFSQEEWACLNSAQRDLYWDVMLENYSNL |
| | | VSLDLESAYENKSLPTEKNIHEIRASKRNSDRRSKSLGR |
| 205 | Z354C_HUMAN | AVDLLSAQEPVTFRDVAVFFSQDEWLHLDSAQRALYREVML |
| | | ENYSSLVSLGIPFSMPKLIHQLQQGEDPCMVEREVPSDT |
| 206 | ZN157_HUMAN | SPQRFPALIPGEPGRSFEGSVSFEDVAVDFTRQEWHRLDPAQR |
| | | TMHKDVMLETYSNLASVGLCVAKPEMIFKLERGEELW |
| 207 | ZN727_HUMAN | RVLTFRDVAVEFSPEEWECLDSAQQRLYRDVMLENYGNLFSL |
| | | GLAIFKPDLITYLEQRKEPWNARRQKTVAKHPAGSLHF |
| 208 | ZN550_HUMAN | AETKDAAQMLVTFKDVAVTFTREEWRQLDLAQRTLYREVML |
| | | ETCGLLVSLGHRVPKPELVHLLEHGQELWIVKRGLSHAT |
| 209 | ZN793_HUMAN | IEYQIPVSFKDVVVGFTQEEWHRLSPAQRALYRDVMLETYSN |
| | | LVSVGYEGTKPDVILRLEQEEAPWIGEAACPGCHCWED |
| 210 | ZN235_HUMAN | TKFQEAVTFKDVAVAFTEEELGLLDSAQRKLYRDVMLENFRN |
| | | LVSVGHQSFKPDMISQLEREEKLWMKELQTQRGKHSGD |
| 211 | ZNF8_HUMAN | DEGVAGVMSVGPPAARLQEPVTFRDVAVDFTQEEWGQLDPT |
| | | QRILYRDVMLETFGHLLSIGPELPKPEVISQLEQGTELW |
| 212 | ZN724_HUMAN | GPLTFMDVAIEFSVEEWQCLDTAQQNLYRNVMLENYRNLVF |
| | | LGIAVSKPDLITCLEQGKEPWNMERHEMVAKPPGMCCYF |
| 213 | ZN573_HUMAN | HQVGLIRSYNSKTMTCFQELVTFRDVAIDFSRQEWEYLDPNQ |
| | | RDLYRDVMLENYRNLVSLGGHSISKPVVVDLLERGKEP |
| 214 | ZN577_HUMAN | NATIVMSVRREQGSSSGEGSLSFEDVAVGFTREEWQFLDQSQ |
| | | KVLYKEVMLENYINLVSIGYRGTKPDSLFKLEQGEPPG |
| 215 | ZN789_HUMAN | FPPARGKELLSFEDVAMYFTREEWGHLNWGQKDLYRDVMLE |
| | | NYRNMVLLGFQFPKPEMICQLENWDEQWILDLPRTGNRK |
| 216 | ZN718_HUMAN | ELLTFKDVAIEFSPEEWKCLDTSQQNLYRDVMLENYRNLVSL |
| | | GVSISNPDLVTSLEQRKEPYNLKIHETAARPPAVCSHF |
| 217 | ZN300_HUMAN | MKSQGLVSFKDVAVDFTQEEWQQLDPSQRTLYRDVMLENYS |
| | | HLVSMGYPVSKPDVISKLEQGEEPWIIKGDISNWIYPDE |
| 218 | ZN383_HUMAN | AEGSVMFSDVSIDFSQEEWDCLDPVQRDLYRDVMLENYGNL |
| | | VSMGLYTPKPQVISLLEQGKEPWMVGRELTRGLCSDLES |
| 219 | ZN429_HUMAN | GPLTFTDVAIEFSLEEWQCLDTAQQNLYRNVMLENYRNLVFL |
| | | GIAVSKPDLITCLEKEKEPCKMKRHEMVDEPPVVCSHF |
| 220 | ZN677_HUMAN | ALSQGLFTFKDVAIEFSQEEWECLDPAQRALYRDVMLENYRN |
| | | LLSLDEDNIPPEDDISVGFTSKGLSPKENNKEELYHLV |
| 221 | ZN850_HUMAN | NMEGLVMFQDLSIDFSQEEWECLDAAQKDLYRDVMMENYSS |
| | | LVSLGLSIPKPDVISLLEQGKEPWMVSRDVLGGWCRDSE |
| 222 | ZN454_HUMAN | AVSHLPTMVQESVTFKDVAILFTQEEWGQLSPAQRALYRDV |
| | | MLENYSNLVSLGLLGPKPDTFSQLEKREVWMPEDTPGGF |
| 223 | ZN257_HUMAN | GPLTIRDVTVEFSLEEWHCLDTAQQNLYRDVMLENYRNLVFL |
| | | GIAVSKPDLITCLEQGKEPCNMKRHEMVAKPPVMCSHI |
| 224 | ZN264_HUMAN | AAAVLTDRAQVSVTFDDVAVTFTKEEWGQLDLAQRTLYQEV |
| | | MLENCGLLVSLGCPVPKAELICHLEHGQEPWTRKEDLSQ |
| 225 | ZFP82_HUMAN | ALRSVMFSDVSIDFSPEEWEYLDLEQKDLYRDVMLENYSNLV |
| | | SLGCFISKPDVISSLEQGKEPWKVVRKGRRQYPDLETK |
| 226 | ZFP14_HUMAN | AHGSVTFRDVAIDFSQEEWEFLDPAQRDLYRDVMWENYSNFI |
| | | SLGPSISKPDVITLLDEERKEPGMVVREGTRRYCPDLE |
| 227 | ZN485_HUMAN | APRAQIQGPLTFGDVAVAFTRIEWRHLDAAQRALYRDVMLE |
| | | NYGNLVSVGLLSSKPKLITQLEQGAEPWTEVREAPSGTH |
| 228 | ZN737_HUMAN | GPLQFRDVAIEFSLEEWHCLDTAQRNLYRNVMLENYRNLVFL |
| | | GIVVSKPDLITCLEQGKKPLTMKKHEMVANPSVTCSHF |
| 229 | ZNF44_HUMAN | TLPRGQPEVLEWGLPKDQDSVAFEDVAVNFTHEEWALLGPSQ |
| | | KNLYRDVMRETIRNLNCIGMKWENQNIDDQHQNLRRNP |
| 230 | ZN596_HUMAN | PSPDSMTFEDIIVDFTQEEWALLDTSQRKLFQDVMLENISHLV |
| | | SIGKQLCKSVVLSQLEQVEKLSTQRISLLQGREVGIK |
| 231 | ZN565_HUMAN | EESREIRAGQIVLKAMAQGLVTFRDVAIEFSLEEWKCLEPAQR |
| | | DLYREVTLENFGHLASLGLSISKPDVVSLLEQGKEPW |
| 232 | ZN543_HUMAN | AASAQVSVTFEDVAVTFTQEEWGQLDAAQRTLYQEVMLETC |
| | | GLLMSLGCPLFKPELIYQLDHRQELWMATKDLSQSSYPG |
| 233 | ZFP69_HUMAN | RESLEDEVTPGLPTAESQELLTFKDISIDFTQEEWGQLAPAHQN |
| | | LYREVMLENYSNLVSVGYQLSKPSVISQLEKGEEPW |
| 234 | SUMO1_HUMAN | EGEYIKLKVIGQDSSEIHFKVKMTTHLKKLKESYCQRQGVPM |
| | | NSLRFLFEGQRIADNHTPKELGMEEEDVIEVYQEQTGG |
| 235 | ZNF12_HUMAN | NKSLGPVSFKDVAVDFTQEEWQQLDPEQKITYRDVMLENYS |
| | | NLVSVGYHIIKPDVISKLEQGEEPWIVEGEFLLQSYPDE |
| 236 | ZN169_HUMAN | SPGLLTTRKEALMAFRDVAVAFTQKEWKLLSSAQRTLYREV |
| | | MLENYSHLVSLGIAFSKPKLIEQLEQGDEPWREENEHLL |
| 237 | ZN433_HUMAN | MFQDSVAFEDVAVTFTQEEWALLDPSQKNLCRDVMQETFRN |
| | | LASIGKKWKPQNIYVEYENLRRNLRIVGERLFESKEGHQ |
| 238 | SUMO3_HUMAN | ENDHINLKVAGQDGSVVQFKIKRHTPLSKLMKAYCERQGLS |
| | | MRQIRFRFDGQPINETDTPAQLEMEDEDTIDVFQQQTGG |
| 239 | ZNF98_HUMAN | PGPLGSLEMGVLTFRDVALEFSLEEWQCLDTAQQNLYRNVM |
| | | LENYRNLVFVGIAASKPDLITCLEQGKEPWNVKRHEMVT |
| 240 | ZN175_HUMAN | LSQKPQVLGPEKQDGSCEASVSFEDVTVDFSREEWQQLDPAQ |
| | | RCLYRDVMLELYSHLFAVGYHIPNPEVIFRMLKEKEPR |
| 241 | ZN347_HUMAN | ALTQGQVTFRDVAIEFSQEEWTCLDPAQRTLYRDVMLENYRN |
| | | LASLGISCFDLSIISMLEQGKEPFTLESQVQIAGNPDG |
| 242 | ZNF25_HUMAN | NKFQGPVTLKDVIVEFTKEEWKLLTPAQRTLYKDVMLENYSH |
| | | LVSVGYHVNKPNAVFKLKQGKEPWILEVEFPHRGFPED |
| 243 | ZN519_HUMAN | ELLTFRDVAIEFSPEEWKCLDPAQQNLYRDVMLENYRNLVSL |
| | | AVYSYYNQGILPEQGIQDSFKKATLGRYGSCGLENICL |
| 244 | Z585B_HUMAN | SPQKSSALAPEDHGSSYEGSVSFRDVAIDFSREEWRHLDLSQR |
| | | NLYRDVMLETYSHLLSVGYQVPKPEVVMLEQGKEPWA |
| 245 | ZIM3_HUMAN | NNSQGRVTFEDVTVNFTQGEWQRLNPEQRNLYRDVMLENYS |
| | | NLVSVGQGETTKPDVILRLEQGKEPWLEEEEVLGSGRAE |
| 246 | ZN517_HUMAN | AMALPMPGPQEAVVFEDVAVYFTRIEWSCLAPDQQALYRDV |
| | | MLENYGNLASLGFLVAKPALISLLEQGEEPGALILQVAE |
| 247 | ZN846_HUMAN | DSSQHLVTFEDVAVDFTQEEWTLLDQAQRDLYRDVMLENYK |
| | | NLIILAGSELFKRSLMSGLEQMEELRTGVTGVLQELDLQ |
| 248 | ZN230_HUMAN | TTFKEAVTFKDVAVFFTEEELGLLDPAQRKLYQDVMLENFTN |
| | | LLSVGHQPFHPFHFLREEKFWMMETATQREGNSGGKTI |
| 249 | ZNF66_HUMAN | GPLQFRDVAIEFSLEEWHCLDMAQRNLYRDVMLENYRNLVF |
| | | LGIVVSKPDLITHLEQGKKPSTMQRHEMVANPSVLCSHF |
| 250 | ZFP1_HUMAN | NKSQGSVSFTDVTVDFTQEEWEQLDPSQRILYMDVMLENYSN |
| | | LLSVEVWKADDQMERDHRNPDEQARQFLILKNQTPIEE |
| 251 | ZN713_HUMAN | EEEEMNDGSQMVRSQESLTFQDVAVDFTREEWDQLYPAQKN |
| | | LYRDVMLENYRNLVALGYQLCKPEVIAQLELEEEWVIER |
| 252 | ZN816_HUMAN | EEATKKSKEKEPGMALPQGRLTFRDVAIEFSLEEWKCLNPAQ |
| | | RALYRAVMLENYRNLEFVDSSLKSMMEFSSTRHSITGE |
| 253 | ZN426_HUMAN | EKTPAGRIVADCLTDCYQDSVTFDDVAVDFTQEEWTLLDSTQ |
| | | RSLYSDVMLENYKNLATVGGQIIKPSLISWLEQEESRT |
| 254 | ZN674_HUMAN | AMSQESLTFKDVFVDFTLEEWQQLDSAQKNLYRDVMLENYS |
| | | HLVSVGHLVGKPDVIFRLGPGDESWMADGGTPVRTCAGE |
| 255 | ZN627_HUMAN | DSVAFEDVAVNFTLEEWALLDPSQKNLYRDVMRETFRNLAS |
| | | VGKQWEDQNIEDPFKIPRRNISHIPERLCESKEGGQGEE |
| 256 | ZNF20_HUMAN | MFQDSVAFEDVAVSFTQEEWALLDPSQKNLYRDVMQETFKN |
| | | LTSVGKTWKVQNIEDEYKNPRRNLSLMREKLCESKESHH |
| 257 | Z587B_HUMAN | AVVATLRLSAQGTVTFEDVAVKFTQEEWNLLSEAQRCLYRD |
| | | VTLENLALMSSLGCWCGVEDEAAPSKQSIYIQRETQVRT |
| 258 | ZN316_HUMAN | EEEEEDEDEDDLLTAGCQELVTFEDVAVYFSLEEWERLEADQ |
| | | RGLYQEVMQENYGILVSLGYPIPKPDLIFRLEQGEEPW |
| 259 | ZN233_HUMAN | TKFQEMVTFKDVAVVFTREELGLLDLAQRKLYQDVMLENFR |
| | | NLLSVGYQPFKLDVILQLGKEDKLRMMETEIQGDGCSGH |
| 260 | ZN611_HUMAN | EEAAQKRKGKEPGMALPQGRLTFRDVAIEFSLAEWKCLNPSQ |
| | | RALYREVMLENYRNLEAVDISSKCMMKEVLSTGQGNTE |
| 261 | ZN556_HUMAN | DTVVFEDVVVDFTLEEWALLNPAQRKLYRDVMLETFKHLAS |
| | | VDNEAQLKASGSISQQDTSGEKLSLKQKIEKFTRKNIWA |
| 262 | ZN234_HUMAN | TTFKEGLTFKDVAVVFTEEELGLLDPVQRNLYQDVMLENFRN |
| | | LLSVGHHPFKHDVFLLEKEKKLDIMKTATQRKGKSADK |
| 263 | ZN560_HUMAN | SALQQEFWKIQTSNGIQMDLVTFDSVAVEFTQEEWTLLDPAQ |
| | | RNLYSDVMLENYKNLSSVGYQLFKPSLISWLEEEEELS |
| 264 | ZNF77_HUMAN | DCVIFEEVAVNFTPEEWALLDHAQRSLYRDVMLETCRNLASL |
| | | DCYIYVRTSGSSSQRDVFGNGISNDEEIVKFTGSDSWS |
| 265 | ZN682_HUMAN | ELLTFRDVTIEFSLEEWEFLNPAQQSLYRKVMLENYRNLVSLG |
| | | LTVSKPELISRLEQRQEPWNVKRHETIAKPPAMSSHY |
| 266 | ZN614_HUMAN | IKTQESLTLEDVAVEFSWEEWQLLDTAQKNLYRDVMVENYN |
| | | HLVSLGYQTSKPDVLSKLAHGQEPWTTDAKIQNKNCPGI |
| 267 | ZN785_HUMAN | PAHVPGEAGPRRTRESRPGAVSFADVAVYFSPEEWECLRPAQ |
| | | RALYRDVMRETFGHLGALGFSVPKPAFISWVEGEVEAW |
| 268 | ZN445_HUMAN | GCPGDQVTPTRSLTAQLQETMTFKDVEVTFSQDEWGWLDSA |
| | | QRNLYRDVMLENYRNMASLVGPFTKPALISWLEAREPWG |
| 269 | ZFP30_HUMAN | ARDLVMFRDVAVDFSQEEWECLNSYQRNLYRDVILENYSNL |
| | | VSLAGCSISKPDVITLLEQGKEPWMVVRDEKRRWTLDLE |
| 270 | ZN225_HUMAN | TTLKEAVTFKDVAVVFTEEELRLLDLAQRKLYREVMLENFRN |
| | | LLSVGHQSLHRDTFHFLKEEKFWMMETATQREGNLGGK |
| 271 | ZN551_HUMAN | SPPSPRSSMAAVALRDSAQGMTFEDVAIYFSQEEWELLDESQR |
| | | FLYCDVMLENFAHVTSLGYCHGMENEAIASEQSVSIQ |
| 272 | ZN610_HUMAN | DEEAQKRKAKESGMALPQGRLTFMDVAIEFSQEEWKSLDPG |
| | | QRALYRDVMLENYRNLVFLGICLPDLSIISMLKQRREPL |
| 273 | ZN528_HUMAN | ALTQGPLKFMDVAIEFSQEEWKCLDPAQRTLYRDVMLENYR |
| | | NLVSLGICLPDLSVTSMLEQKRDPWTLQSEEKIANDPDG |
| 274 | ZN284_HUMAN | TMFKEAVTFKDVAVVFTEEELGLLDVSQRKLYRDVMLENFR |
| | | NLLSVGHQLSHRDTFHFQREEKFWIMETATQREGNSGGK |
| 275 | ZN418_HUMAN | QGTVAFEDVAVNFSQEEWSLLSEVQRCLYHDVMLENWVLISS |
| | | LGCWCGSEDEEAPSKKSISIQRVSQVSTPGAGVSPKKA |
| 276 | MPP8_HUMAN | AEAFGDSEEDGEDVFEVEKILDMKTEGGKVLYKVRWKGYTS |
| | | DDDTWEPEIHLEDCKEVLLEFRKKIAENKAKAVRKDIQR |
| 277 | ZN490_HUMAN | VLQMQNSEHHGQSIKTQTDSISLEDVAVNFTLEEWALLDPGQ |
| | | RNIYRDVMRATFKNLACIGEKWKDQDIEDEHKNQGRNL |
| 278 | ZN805_HUMAN | AMALTDPAQVSVTFDDVAVTFTQEEWGQLDLAQRTLYQEV |
| | | MLENCGLLVSLGCPVPRPELIYHLEHGQEPWTRKEDLSQG |
| 279 | Z780B_HUMAN | VHGSVTFRDVAIDFSQEEWECLQPDQRTLYRDVMLENYSHLI |
| | | SLGSSISKPDVITLLEQEKEPWIVVSKETSRWYPDLES |
| 280 | ZN763_HUMAN | DPVACEDVAVNFTQEEWALLDISQRKLYREVMLETFRNLTSI |
| | | GKKWKDQNIEYEYQNPRRNFRSLIEGNVNEIKEDSHCG |
| 281 | ZN285_HUMAN | IKFQERVTFKDVAVVFTKEELALLDKAQINLYQDVMLENFRN |
| | | LMLVRDGIKNNILNLQAKGLSYLSQEVLHCWQIWKQRI |
| 282 | ZNF85_HUMAN | GPLTFRDVAIEFSLKEWQCLDTAQRNLYRNVMLENYRNLVFL |
| | | GITVSKPDLITCLEQGKEAWSMKRHEIMVAKPTVMCSH |
| 283 | ZN223_HUMAN | TMSKEAVTFKDVAVVFTEEELGLLDLAQRKLYRDVMLENFR |
| | | NLLSVGHQPFHRDTFHFLREEKFWMMDIATQREGNSGGK |
| 284 | ZNF90_HUMAN | GPLEFRDVAIEFSLEEWHCLDTAQQNLYRDVMLENYRHLVFL |
| | | GIVVTKPDLITCLEQGKKPFTVKRHEMIAKSPVMCFHF |
| 285 | ZN557_HUMAN | GHTEGGELVNELLKSWLKGLVTFEDVAVEFTQEEWALLDPA |
| | | QRTLYRDVMLENCRNLASLGNQVDKPRLISQLEQEDKVM |
| 286 | ZN425_HUMAN | AEPASVTVTFDDVALYFSEQEWEILEKWQKQMYKQEMKTNY |
| | | ETLDSLGYAFSKPDLITWMEQGRMLLISEQGCLDKTRRT |
| 287 | ZN229_HUMAN | HSQASAISQDREEKIMSQEPLSFKDVAVVFTEEELELLDSTQR |
| | | QLYQDVMQENFRNLLSVGERNPLGDKNGKDTEYIQDE |
| 288 | ZN606_HUMAN | GSLEEGRRATGLPAAQVQEPVTFKDVAVDFTQEEWGQLDLV |
| | | QRTLYRDVMLETYGHLLSVGNQIAKPEVISLLEQGEEPW |
| 289 | ZN155_HUMAN | TTFKEAVTFKDVAVVFTEEELGLLDPAQRKLYRDVMLENFRN |
| | | LLSVGHQPFHQDTCHFLREEKFWMMGTATQREGNSGGK |
| 290 | ZN222_HUMAN | AKLYEAVTFKDVAVIFTEEELGLLDPAQRKLYRDVMLENFRN |
| | | LLSVGGKIQTEMETVPEAGTHEEFSCKQIWEQIASDLT |
| 291 | ZN442_HUMAN | RSDLFLPDSQTNEERKQYDSVAFEDVAVNFTQEEWALLGPSQ |
| | | KSLYRDVMWETIRNLDCIGMKWEDTNIEDQHRNPRRSL |
| 292 | ZNF91_HUMAN | PGTPGSLEMGLLTFRDVAIEFSPEEWQCLDTAQQNLYRNVML |
| | | ENYRNLAFLGIALSKPDLITYLEQGKEPWNMKQHEMVD |
| 293 | ZN135_HUMAN | TPGVRVSTDPEQVTFEDVVVGFSQEEWGQLKPAQRTLYRDV |
| | | MLDTFRLLVSVGHWLPKPNVISLLEQEAELWAVESRLPQ |
| 294 | ZN778_HUMAN | EQTQAAGMVAGWLINCYQDAVTFDDVAVDFTQEEWTLLDPS |
| | | QRDLYRDVMLENYENLASVEWRLKTKGPALRQDRSWFRA |
| 295 | RYBP_HUMAN | PSEANSIQSANATTKTSETNHTSRPRLKNVDRSTAQQLAVTVG |
| | | NVTVIITDFKEKTRSSSTSSSTVTSSAGSEQQNQSSS |
| 296 | ZN534_HUMAN | ALTQGQLSFSDVAIEFSQEEWKCLDPGQKALYRDVMLENYRN |
| | | LVSLGEDNVRPEACICSGICLPDLSVTSMLEQKRDPWT |
| 297 | ZN586_HUMAN | AAAAALRAPAQSSVTFEDVAVNFSLEEWSLLNEAQRCLYRDV |
| | | MLETLTLISSLGCWHGGEDEAAPSKQSTCIHIYKDQGG |
| 298 | ZN567_HUMAN | AQGSVSFNDVTVDFTQEEWQHLDHAQKTLYMDVMLENYCH |
| | | LISVGCHMTKPDVILKLERGEEPWTSFAGHTCLEENWKAE |
| 299 | ZN440_HUMAN | DPVAFKDVAVNFTQEEWALLDISQRKLYREVMLETFRNLTSL |
| | | GKRWKDQNIEYEHQNPRRNFRSLIEEKVNEIKDDSHCG |
| 300 | ZN583_HUMAN | SKDLVTFGDVAVNFSQEEWEWLNPAQRNLYRKVMLENYRSL |
| | | VSLGVSVSKPDVISLLEQGKEPWMVKKEGTRGPCPDWEY |
| 301 | ZN441_HUMAN | DSVAFEDVAINFTCEEWALLGPSQKSLYRDVMQETIRNLDCIG |
| | | MIWQNHDIEEDQYKDLRRNLRCHMVERACEIKDNSQC |
| 302 | ZNF43_HUMAN | GPLTFMDVAIEFCLEEWQCLDIAQQNLYRNVMLENYRNLVFL |
| | | GIAVSKPDLITCLEQEKEPWEPMRRHEMVAKPPVMCSH |
| 303 | CBX5_HUMAN | QSNDIARGFERGLEPEKIIGATDSCGDLMFLMKWKDTDEADL |
| | | VLAKEANVKCPQIVIAFYEERLTWHAYPEDAENKEKET |
| 304 | ZN589_HUMAN | ALPAKDSAWPWEEKPRYLGPVTFEDVAVLFTEAEWKRLSLE |
| | | QRNLYKEVMLENLRNLVSLAESKPEVHTCPSCPLAFGSQ |
| 305 | ZNF10_HUMAN | DAKSLTAWSRTLVTFKDVFVDFTREEWKLLDTAQQIVYRNV |
| | | MLENYKNLVSLGYQLTKPDVILRLEKGEEPWLVEREIHQ |
| 306 | ZN563_HUMAN | DAVAFEDVAVNFTQEEWALLGPSQKNLYRYVMQETIRNLDCI |
| | | RMIWEEQNTEDQYKNPRRNLRCHMVERFSESKDSSQCG |
| 307 | ZN561_HUMAN | EKTKVERMVEDYLASGYQDSVTFDDVAVDFTPEEWALLDTT |
| | | EKYLYRDVMLENYMNLASVEWEIQPRTKRSSLQQGFLKN |
| 308 | ZN136_HUMAN | DSVAFEDVDVNFTQEEWALLDPSQKNLYRDVMWETMRNLA |
| | | SIGKKWKDQNIKDHYKHRGRNLRSHMLERLYQTKDGSQRG |
| 309 | ZN630_HUMAN | IESQEPVTFEDVAVDFTQEEWQQLNPAQKTLHRDVMLETYNH |
| | | LVSVGCSGIKPDVIFKLEHGKDPWIIESELSRWIYPDR |
| 310 | ZN527_HUMAN | AVGLCKAMSQGLVTFRDVALDFSQEEWEWLKPSQKDLYRDV |
| | | MLENYRNLVWLGLSISKPNMISLLEQGKEPWMVERKMSQ |
| 311 | ZN333_HUMAN | DKVEEEAMAPGLPTACSQEPVTFADVAVVFTPEEWVFLDSTQ |
| | | RSLYRDVMLENYRNLASVADQLCKPNALSYLEERGEQW |
| 312 | Z324B_HUMAN | TFEDVAVYFSQEEWGLLDTAQRALYRHVMLENFTLVTSLGLS |
| | | TSRPRVVIQLERGEEPWVPSGKDMTLARNTYGRLNSGS |
| 313 | ZN786_HUMAN | AEPPRLPLTFEDVAIYFSEQEWQDLEAWQKELYKHVMRSNYE |
| | | TLVSLDDGLPKPELISWIEHGGEPFRKWRESQKSGNII |
| 314 | ZN709_HUMAN | DSVVFEDVAVNFTQEEWALLGPSQKKLYRDVMQETFVNLASI |
| | | GENWEEKNIEDHKNQGRKLRSHMVERLCERKEGSQFGE |
| 315 | ZN792_HUMAN | AAAALRDPAQGCVTFEDVTIYFSQEEWVLLDEAQRLLYCDV |
| | | MLENFALIASLGLISFRSHIVSQLEMGKEPWVPDSVDMT |
| 316 | ZN599_HUMAN | AAPALALVSFEDVVVTFTGEEWGHLDLAQRTLYQEVMLETC |
| | | RLLVSLGHPVPKPELIYLLEHGQELWTVKRGLSQSTCAG |
| 317 | ZN613_HUMAN | IKSQESLTLEDVAVEFTWEEWQLLGPAQKDLYRDVMLENYS |
| | | NLVSVGYQASKPDALFKLEQGEPWTVENEIHSQICPEIK |
| 318 | ZF69B_HUMAN | GESLESRVTLGSLTAESQELLTFKDVSVDFTQEEWGQLAPAHR |
| | | NLYREVMLENYGNLVSVGCQLSKPGVISQLEKGEEPW |
| 319 | ZN799_HUMAN | ASVALEDVAVNFTREEWALLGPCQKNLYKDVMQETIRNLDC |
| | | VGMKWKDQNIEDQYRYPRKNLRCRMLERFVESKDGTQCG |
| 320 | ZN569_HUMAN | TESQGTVTFKDVAIDFTQEEWKRLDPAQRKLYRNVMLENYN |
| | | NLITVGYPFTKPDVIFKLEQEEEPWVMEEEVLRRHWQGE |
| 321 | ZN564_HUMAN | DSVASEDVAVNFTLEEWALLDPSQKKLYRDVMRETFRNLAC |
| | | VGKKWEDQSIEDWYKNQGRILRNHMEEGLSESKEYDQCG |
| 322 | ZN546_HUMAN | EETQGELTSSCGSKTMANVSLAFRDVSIDLSQEEWECLDAVQ |
| | | RDLYKDVMLENYSNLVALGYTIPKPDVITLLEQEKEPW |
| 323 | ZFP92_HUMAN | AAILLTTRPKVPVSFEDVSVYFTKTEWKLLDLRQKVLYKRVM |
| | | LENYSHLVSLGFSFSKPHLISQLERGEGPWVADIPRTW |
| 324 | YAF2_HUMAN | KDKVEKEKSEKETTSKKNSHKKTRPRLKNVDRSSAQHLEVTV |
| | | GDLTVIITDFKEKTKSPPASSAASADQHSQSGSSSDNT |
| 325 | ZN723_HUMAN | GPLTFTDVAIKFSLEEWQFLDTAQQNLYRDVMLENYRNLVFL |
| | | GVGVSKPDLITCLEQGKEPWNMKRHKMVAKPPVVCSHF |
| 326 | ZNF34_HUMAN | RKPNPQAMAALFLSAPPQAEVTFEDVAVYLSREEWGRLGPAQ |
| | | RGLYRDVMLETYGNLVSLGVGPAGPKPGVISQLERGDE |
| 327 | ZN439_HUMAN | LSLSPILLYTCEMFQDPVAFKDVAVNFTQEEWALLDISQKNLY |
| | | REVMLETFWNLTSIGKKWKDQNIEYEYQNPRRNFRSV |
| 328 | ZFP57_HUMAN | AAGEPRSLLFFQKPVTFEDVAVNFTQEEWDCLDASQRVLYQD |
| | | VMSETFKNLTSVARIFLHKPELITKLEQEEEQWRETRV |
| 329 | ZNF19_HUMAN | AAMPLKAQYQEMVTFEDVAVHFTKTEWTGLSPAQRALYRSV |
| | | MLENFGNLTALGYPVPKPALISLLERGDMAWGLEAQDDP |
| 330 | ZN404_HUMAN | ARVPLTFSDVAIDFSQEEWEYLNSDQRDLYRDVMLENYTNLV |
| | | SLDFNFTTESNKLSSEKRNYEVNAYHQETWKRNKTFNL |
| 331 | ZN274_HUMAN | ASRLPTAWSCEPVTFEDVTLGFTPEEWGLLDLKQKSLYREVM |
| | | LENYRNLVSVEHQLSKPDVVSQLEEAEDFWPVERGIPQ |
| 332 | CBX3_HUMAN | SKKKRDAADKPRGFARGLDPERIIGATDSSGELMFLMKWKDS |
| | | DEADLVLAKEANMKCPQIVIAFYEERLTWHSCPEDEAQ |
| 333 | ZNF30_HUMAN | AHKYVGLQYHGSVTFEDVAIAFSQQEWESLDSSQRGLYRDV |
| | | MLENYRNLVSMGHSRSKPHVIALLEQWKEPEVTVRKDGR |
| 334 | ZN250_HUMAN | AAARLLPVPAGPQPLSFQAKLTFEDVAVLLSQDEWDRLCPAQ |
| | | RGLYRNVMMETYGNVVSLGLPGSKPDIISQLERGEDPW |
| 335 | ZN570_HUMAN | AVGLLKAMYQELVTFRDVAVDFSQEEWDCLDSSQRHLYSNV |
| | | MLENYRILVSLGLCFSKPSVILLLEQGKAPWMVKRELTK |
| 336 | ZN675_HUMAN | GLLTFRDVAIEFSLEEWQCLDTAQRNLYKNVILENYRNLVFLG |
| | | IAVSKQDLITCLEQEKEPLTVKRHEMVNEPPVMCSHF |
| 337 | ZN695_HUMAN | GLLAFRDVALEFSPEEWECLDPAQRSLYRDVMLENYRNLISL |
| | | GEDSFNMQFLFHSLAMSKPELIICLEARKEPWNVNTEK |
| 338 | ZN548_HUMAN | NLTEGRVVFEDVAIYFSQEEWGHLDEAQRLLYRDVMLENLA |
| | | LLSSLGSWHGAEDEEAPSQQGFSVGVSEVTASKPCLSSQ |
| 339 | ZN132_HUMAN | GPAQHTSWPCGSAVPTLKSMVTFEDVAVYFSQEEWELLDAA |
| | | QRHLYHSVMLENLELVTSLGSWHGVEGEGAHPKQNVSVE |
| 340 | ZN738_HUMAN | SGYPGAERNLLEYSYFEKGPLTFRDVVIEFSQEEWQCLDTAQQ |
| | | DLYRKVMLENFRNLVFLGIDVSKPDLITCLEQGKDPW |
| 341 | ZN420_HUMAN | ARKLVMFRDVAIDFSQEEWECLDSAQRDLYRDVMLENYSNL |
| | | VSLDLPSRCASKDLSPEKNTYETELSQWEMSDRLENCDL |
| 342 | ZN626_HUMAN | GPLQFRDVAIEFSLEEWHCLDTAQRNLYRNVMLENYSNLVFL |
| | | GITVSKPDLITCLEQGRKPLTMKRNEMIAKPSVMCSHF |
| 343 | ZN559_HUMAN | VAGWLTNYSQDSVTFEDVAVDFTQEEWTLLDQTQRNLYRDV |
| | | MLENYKNLVAVDWESHINTKWSAPQQNFLQGKTSSVVEM |
| 344 | ZN460_HUMAN | AAAWMAPAQESVTFEDVAVTFTQEEWGQLDVTQRALYVEV |
| | | MLETCGLLVALGDSTKPETVEPIPSHLALPEEVSLQEQLA |
| 345 | ZN268_HUMAN | VLEWLFISQEQPKITKSWGPLSFMDVFVDFTWEEWQLLDPAQ |
| | | KCLYRSVMLENYSNLVSLGYQHTKPDIIFKLEQGEELC |
| 346 | ZN304_HUMAN | AAAVLMDRVQSCVTFEDVFVYFSREEWELLEEAQRFLYRDV |
| | | MLENFALVATLGFWCEAEHEAPSEQSVSVEGVSQVRTAE |
| 347 | ZIM2_HUMAN | AGSQFPDFKHLGTFLVFEELVTFEDVLVDFSPEELSSLSAAQR |
| | | NLYREVMLENYRNLVSLGHQFSKPDIISRLEEEESYA |
| 348 | ZN605_HUMAN | IQSQISFEDVAVDFTLEEWQLLNPTQKNLYRDVMLENYSNLV |
| | | FLEVWLDNPKMWLRDNQDNLKSMERGHKYDVFGKIFNS |
| 349 | ZN844_HUMAN | DLVAFEDVAVNFTQEEWSLLDPSQKNLYREVMQETLRNLASI |
| | | GEKWKDQNIEDQYKNPRNNLRSLLGERVDENTEENHCG |
| 350 | SUMO5_HUMAN | KDEDIKLRVIGQDSSEIHFKVKMTTPLKKLKKSYCQRQGVPV |
| | | NSLRFLFEGQRIADNHTPEELGMEEEDVIEVYQEQIGG |
| 351 | ZN101_HUMAN | DSVAFEDVAVNFTQEEWALLSPSQKNLYRDVTLETFRNLASV |
| | | GIQWKDQDIENLYQNLGIKLRSLVERLCGRKEGNEHRE |
| 352 | ZN783_HUMAN | RNFWILRLPPGSKGEAPKVPVTFDDVAVYFSELEWGKLEDWQ |
| | | KELYKHVMRGNYETLVSLDYAISKPDILTRIERGEEPC |
| 353 | ZN417_HUMAN | AAAAPRRPTQQGTVTFEDVAVNFSQEEWCLLSEAQRCLYRD |
| | | VMLENLALISSLGCWCGSKDEEAPCKQRISVQRESQSRT |
| 354 | ZN182_HUMAN | SGEDSGSFYSWQKAKREQGLVTFEDVAVDFTQEEWQYLNPP |
| | | QRTLYRDVMLETYSNLVFVGQQVTKPNLILKLEVEECPA |
| 355 | ZN823_HUMAN | DSVAFEDVAVNFTQEEWALLGPSQKSLYRNVMQETIRNLDCI |
| | | EMKWEDQNIGDQCQNAKRNLRSHTCEIKDDSQCGETFG |
| 356 | ZN177_HUMAN | AAGWLTTWSQNSVTFQEVAVDFSQEEWALLDPAQKNLYKD |
| | | VMLENFRNLASVGYQLCRHSLISKVDQEQLKTDERGILQG |
| 357 | ZN197_HUMAN | ENPRNQLMALMLLTAQPQELVMFEEVSVCFTSEEWACLGPIQ |
| | | RALYWDVMLENYGNVTSLEWETMTENEEVTSKPSSSQR |
| 358 | ZN717_HUMAN | LETYNSLVSLQELVSFEEVAVHFTWEEWQDLDDAQRTLYRD |
| | | VMLETYSSLVSLGHCITKPEMIFKLEQGAEPWIVEETPN |
| 359 | ZN669_HUMAN | RHFRRPEPCREPLASPIQDSVAFEDVAVNFTQEEWALLDSSQK |
| | | NLYREVMQETCRNLASVGSQWKDQNIEDHFEKPGKDI |
| 360 | ZN256_HUMAN | AAAELTAPAQGIVTFEDVAVYFSWKEWGLLDEAQKCLYHDV |
| | | MLENLTLTTSLGGSGAGDEEAPYQQSTSPQRVSQVRIPK |
| 361 | ZN251_HUMAN | AATFQLPGHQEMPLTFQDVAVYFSQAEGRQLGPQQRALYRD |
| | | VMLENYGNVASLGFPVPKPELISQLEQGKELWVLNLLGA |
| 362 | CBX4_HUMAN | RSEAGEPPSSLQVKPETPASAAVAVAAAAAPTTTAEKPPAEAQ |
| | | DEPAESLSEFKPFFGNIIITDVTANCLTVTFKEYVTV |
| 363 | PCGF2_HUMAN | HRTTRIKITELNPHLMCALCGGYFIDATTIVECLHSFCKTCIVR |
| | | YLETNKYCPMCDVQVHKTRPLLSIRSDKTLQDIVYK |
| 364 | CDY2_HUMAN | ASQEFEVEAIVDKRQDKNGNTQYLVRWKGYDKQDDTWEPE |
| | | QHLMNCEKCVHDFNRRQTEKQKKLTWTTTSRIFSNNARRR |
| 365 | CDYL2_HUMAN | ASGDLYEVERIVDKRKNKKGKWEYLIRWKGYGSTEDTWEPE |
| | | HHLLHCEEFIDEFNGLHMSKDKRIKSGKQSSTSKLLRDS |
| 366 | HERC2_HUMAN | TLIRKADLENHNKDGGFWTVIDGKVYDIKDFQTQSLTGNSILA |
| | | QFAGEDPVVALEAALQFEDTRESMHAFCVGQYLEPDQ |
| 367 | ZN562_HUMAN | EKTKIGTMVEDHRSNSYQDSVTFDDVAVEFTPEEWALLDTTQ |
| | | KYLYRDVMLENYMNLASVDFFFCLTSEWEIQPRTKRSS |
| 368 | ZN461_HUMAN | AHELVMFRDVAIDVSQEEWECLNPAQRNLYKEVMLENYSNL |
| | | VSLGLSVSKPAVISSLEQGKEPWMVVREETGRWCPGTWK |
| 369 | Z324A_HUMAN | AFEDVAVYFSQEEWGLLDTAQRALYRRVMLDNFALVASLGL |
| | | STSRPRVVIQLERGEEPWVPSGTDTTLSRTTYRRRNPGS |
| 370 | ZN766_HUMAN | AQLRRGHLTFRDVAIEFSQEEWKCLDPVQKALYRDVMLENY |
| | | RNLVSLGICLPDLSIISMMKQRTEPWTVENEMKVAKNPD |
| 371 | ID2_HUMAN | SDHSLGISRSKTPVDDPMSLLYNMNDCYSKLKELVPSIPQNKK |
| | | VSKMEILQHVIDYILDLQIALDSHPTIVSLHHQRPGQ |
| 372 | TOX_HUMAN | KDPNEPQKPVSAYALFFRDTQAAIKGQNPNATFGEVSKIVAS |
| | | MWDGLGEEQKQVYKKKTEAAKKEYLKQLAAYRASLVSK |
| 373 | ZN274_HUMAN | QEEKQEDAAICPVTVLPEEPVTFQDVAVDFSREEWGLLGPTQ |
| | | RTEYRDVMLETFGHLVSVGWETTLENKELAPNSDIPEE |
| 374 | SCMH1_HUMAN | DASRLSGRDPSSWTVEDVMQFVREADPQLGPHADLFRKHEID |
| | | GKALLLLRSDMMMKYMGLKLGPALKLSYHIDRLKQGKF |
| 375 | ZN214_HUMAN | AVTFEDVTIIFTWEEWKFLDSSQKRLYREVMWENYTNVMSV |
| | | ENWNESYKSQEEKFRYLEYENFSYWQGWWNAGAQMYENQ |
| 376 | CBX7_HUMAN | ELSAIGEQVFAVESIRKKRVRKGKVEYLVKWKGWPPKYSTW |
| | | EPEEHILDPRLVMAYEEKEERDRASGYRKRGPKPKRLLL |
| 377 | ID1_HUMAN | GGAGARLPALLDEQQVNVLLYDMNGCYSRLKELVPTLPQNR |
| | | KVSKVEILQHVIDYIRDLQLELNSESEVGTPGGRGLPVR |
| 378 | CREM_HUMAN | VVMAASPGSLHSPQQLAEEATRKRELRLMKNREAAKECRRR |
| | | KKEYVKCLESRVAVLEVQNKKLIEELETLKDICSPKTDY |
| 379 | SCX_HUMAN | GGGPGGRPGREPRQRHTANARERDRTNSVNTAFTALRTLIPTE |
| | | PADRKLSKIETLRLASSYISHLGNVLLAGEACGDGQP |
| 380 | ASCL1_HUMAN | SGFGYSLPQQQPAAVARRNERERNRVKLVNLGFATLREHVPN |
| | | GAANKKMSKVETLRSAVEYIRALQQLLDEHDAVSAAFQ |
| 381 | ZN764_HUMAN | APLPPRDPNGAGPEWREPGAVSFADVAVYFCREEWGCLRPA |
| | | QRALYRDVMRETYGHLSALGIGGNKPALISWVEEEAELW |
| 382 | SCML2_HUMAN | KQGFSKDPSTWSVDEVIQFMKHTDPQISGPLADLFRQHEIDGK |
| | | ALFLLKSDVMMKYMGLKLGPALKLCYYIEKLKEGKYS |
| 383 | TWST1_HUMAN | SGGGSPQSYEELQTQRVMANVRERQRTQSLNEAFAALRKIIPT |
| | | LPSDKLSKIQTLKLAARYIDFLYQVLQSDELDSKMAS |
| 384 | CREB1_HUMAN | IAPGVVMASSPALPTQPAEEAARKREVRLMKNREAARECRRK |
| | | KKEYVKCLENRVAVLENQNKTLIEELKALKDLYCHKSD |
| 385 | TERF1_HUMAN | SRIPVSKSQPVTPEKHRARKRQAWLWEEDKNLRSGVRKYGE |
| | | GNWSKILLHYKFNNRTSVMLKDRWRTMKKLKLISSDSED |
| 386 | ID3_HUMAN | SLAIARGRGKGPAAEEPLSLLDDMNHCYSRLRELVPGVPRGT |
| | | QLSQVEILQRVIDYILDLQVVLAEPAPGPPDGPHLPIQ |
| 387 | CBX8_HUMAN | GSGPPSSGGGLYRDMGAQGGRPSLIARIPVARILGDPEEESWS |
| | | PSLTNLEKVVVTDVTSNFLTVTIKESNTDQGFFKEKR |
| 388 | CBX4_HUMAN | ELPAVGEHVFAVESIEKKRIRKGRVEYLVKWRGWSPKYNTW |
| | | EPEENILDPRLLIAFQNRERQEQLMGYRKRGPKPKPLVV |
| 389 | GSX1_HUMAN | VDSSSNQLPSSKRMRTAFTSTQLLELEREFASNMYLSRLRRIEI |
| | | ATYLNLSEKQVKIWFQNRRVKHKKEGKGSNHRGGGG |
| 390 | NKX22_HUMAN | TPGGGGDAGKKRKRRVLFSKAQTYELERRFRQQRYLSAPERE |
| | | HLASLIRLTPTQVKIWFQNHRYKMKRARAEKGMEVTPL |
| 391 | ATF1_HUMAN | QTVVMTSPVTLTSQTTKTDDPQLKREIRLMKNREAARECRRK |
| | | KKEYVKCLENRVAVLENQNKTLIEELKTLKDLYSNKSV |
| 392 | TWST2_HUMAN | KGSPSAQSFEELQSQRILANVRERQRTQSLNEAFAALRKIIPTL |
| | | PSDKLSKIQTLKLAARYIDFLYQVLQSDEMDNKMTS |
| 393 | ZNF17_HUMAN | NLTEDYMVFEDVAIHFSQEEWGILNDVQRHLHSDVMLENFAL |
| | | LSSVGCWHGAKDEEAPSKQCVSVGVSQVTTLKPALSTQ |
| 394 | TOX3_HUMAN | KDPNEPQKPVSAYALFFRDTQAAIKGQNPNATFGEVSKIVAS |
| | | MWDSLGEEQKQVYKRKTEAAKKEYLKALAAYRASLVSK |
| 395 | TOX4_HUMAN | KDPNEPQKPVSAYALFFRDTQAAIKGQNPNATFGEVSKIVAS |
| | | MWDSLGEEQKQVYKRKTEAAKKEYLKALAAYKDNQECQ |
| 396 | ZMYM3_HUMAN | LDGSTWDFCSEDCKSKYLLWYCKAARCHACKRQGKLLETIH |
| | | WRGQIRHFCNQQCLLRFYSQQNQPNLDTQSGPESLLNSQ |
| 397 | I2BP1_HUMAN | ASVQASRRQWCYLCDLPKMPWAMVWDFSEAVCRGCVNFEG |
| | | ADRIELLIDAARQLKRSHVLPEGRSPGPPALKHPATKDLA |
| 398 | RHXF1_HUMAN | MEGPQPENMQPRTRRTKFTLLQVEELESVFRHTQYPDVPTRR |
| | | ELAENLGVTEDKVRVWFKNKRARCRRHQRELMLANELR |
| 399 | SSX2_HUMAN | PKIMPKKPAEEGNDSEEVPEASGPQNDGKELCPPGKPTTSEKI |
| | | HERSGPKRGEHAWTHRLRERKQLVIYEEISDPEEDDE |
| 400 | I2BPL_HUMAN | SAAQVSSSRRQSCYLCDLPRMPWAMIWDFSEPVCRGCVNYE |
| | | GADRIEFVIETARQLKRAHGCFQDGRSPGPPPPVGVKTV |
| 401 | ZN680_HUMAN | PGPPGSLEMGPLTFRDVAIEFSLEEWQCLDTAQRNLYRKVMF |
| | | ENYRNLVFLGIAVSKPHLITCLEQGKEPWNRKRQEMVA |
| 402 | CBX1_HUMAN | NKKKVEEVLEEEEEEYVVEKVLDRRVVKGKVEYLLKWKGFS |
| | | DEDNTWEPEENLDCPDLIAEFLQSQKTAHETDKSEGGKR |
| 403 | TRI68_HUMAN | LANVVEKVRLLRLHPGMGLKGDLCERHGEKLKMFCKEDVLI |
| | | MCEACSQSPEHEAHSVVPMEDVAWEYKWELHEALEHLKK |
| 404 | HXA13_HUMAN | VVSHPSDASSYRRGRKKRVPYTKVQLKELEREYATNKFITKD |
| | | KRRRISATTNLSERQVTIWFQNRRVKEKKVINKLKTTS |
| 405 | PHC3_HUMAN | ENSDLLPVAQTEPSIWTVDDVWAFIHSLPGCQDIADEFRAQEI |
| | | DGQALLLLKEDHLMSAMNIKLGPALKICARINSLKES |
| 406 | TCF24_HUMAN | AGPGGGSRSGSGRPAAANAARERSRVQTLRHAFLELQRTLPS |
| | | VPPDTKLSKLDVLLLATTYIAHLTRSLQDDAEAPADAG |
| 407 | CBX3_HUMAN | QNGKSKKVEEAEPEEFVVEKVLDRRVVNGKVEYFLKWKGFT |
| | | DADNTWEPEENLDCPELIEAFLNSQKAGKEKDGTKRKSL |
| 408 | HXB13_HUMAN | QHPPDACAFRRGRKKRIPYSKGQLRELEREYAANKFITKDKR |
| | | RKISAATSLSERQITIWFQNRRVKEKKVLAKVKNSATP |
| 409 | HEY1_HUMAN | SMSPTTSSQILARKRRRGIIEKRRRDRINNSLSELRRLVPSAFEK |
| | | QGSAKLEKAEILQMTVDHLKMLHTAGGKGYFDAHA |
| 410 | PHC2_HUMAN | LVGMGHHFLPSEPTKWNVEDVYEFIRSLPGCQEIAEEFRAQEI |
| | | DGQALLLLKEDHLMSAMNIKLGPALKIYARISMLKDS |
| 411 | ZNF81_HUMAN | PANEDAPQPGEHGSACEVSVSFEDVTVDFSREEWQQLDSTQR |
| | | RLYQDVMLENYSHLLSVGFEVPKPEVIFKLEQGEGPWT |
| 412 | FIGLA_HUMAN | GYSSTENLQLVLERRRVANAKERERIKNLNRGFARLKALVPF |
| | | LPQSRKPSKVDILKGATEYIQVLSDLLEGAKDSKKQDP |
| 413 | SAM11_HUMAN | EEAPAPEDVTKWTVDDVCSFVGGLSGCGEYTRVFREQGIDGE |
| | | TLPLLTEEHLLTNMGLKLGPALKIRAQVARRLGRVFYV |
| 414 | KMT2B_HUMAN | GGTLAHTPRRSLPSHHGKKMRMARCGHCRGCLRVQDCGSCV |
| | | NCLDKPKFGGPNTKKQCCVYRKCDKIEARKMERLAKKGR |
| 415 | HEY2_HUMAN | LNSPTTTSQIMARKKRRGIIEKRRRDRINNSLSELRRLVPTAFE |
| | | KQGSAKLEKAEILQMTVDHLKMLQATGGKGYFDAHA |
| 416 | JDP2_HUMAN | QPVKSELDEEEERRKRRREKNKVAAARCRNKKKERTEFLQRE |
| | | SERLELMNAELKTQIEELKQERQQLILMLNRHRPTCIV |
| 417 | HXC13_HUMAN | LQPEVSSYRRGRKKRVPYTKVQLKELEKEYAASKFITKEKRR |
| | | RISATTNLSERQVTIWFQNRRVKEKKVVSKSKAPHLHS |
| 418 | ASCL4_HUMAN | LPVPLDSAFEPAFLRKRNERERQRVRCVNEGYARLRDHLPRE |
| | | LADKRLSKVETLRAAIDYIKHLQELLERQAWGLEGAAG |
| 419 | HHEX_HUMAN | SPFLQRPLHKRKGGQVRFSNDQTIELEKKFETQKYLSPPERKR |
| | | LAKMLQLSERQVKTWFQNRRAKWRRLKQENPQSNKKE |
| 420 | HERC2_HUMAN | IAIATGSLHCVCCTEDGEVYTWGDNDEGQLGDGTTNAIQRPR |
| | | LVAALQGKKVNRVACGSAHTLAWSTSKPASAGKLPAQV |
| 421 | GSX2_HUMAN | GGSDASQVPNGKRMRTAFTSTQLLELEREFSSNMYLSRLRRIE |
| | | IATYLNLSEKQVKIWFQNRRVKHKKEGKGTQRNSHAG |
| 422 | BIN1_HUMAN | RLDLPPGFMFKVQAQHDYTATDTDELQLKAGDVVLVIPFQNP |
| | | EEQDEGWLMGVKESDWNQHKELEKCRGVFPENFTERVP |
| 423 | ETV7_HUMAN | GICKLPGRLRIQPALWSREDVLHWLRWAEQEYSLPCTAEHGF |
| | | EMNGRALCILTKDDFRHRAPSSGDVLYELLQYIKTQRR |
| 424 | ASCL3_HUMAN | PNYRGCEYSYGPAFTRKRNERERQRVKCVNEGYAQLRHHLP |
| | | EEYLEKRLSKVETLRAAIKYINYLQSLLYPDKAETKNNP |
| 425 | PHC1_HUMAN | LHGINPVFLSSNPSRWSVEEVYEFIASLQGCQEIAEEFRSQEIDG |
| | | QALLLLKEEHLMSAMNIKLGPALKICAKINVLKET |
| 426 | OTP_HUMAN | QAGQQQGQQKQKRHRTRFTPAQLNELERSFAKTHYPDIFMRE |
| | | ELALRIGLTESRVQVWFQNRRAKWKKRKKTTNVFRAPG |
| 427 | I2BP2_HUMAN | AAAVAVAAASRRQSCYLCDLPRMPWAMIWDFTEPVCRGCV |
| | | NYEGADRVEFVIETARQLKRAHGCFPEGRSPPGAAASAAA |
| 428 | VGLL2_HUMAN | FSSQTPASIKEEEGSPEKERPPEAEYINSRCVLFTYFQGDISSVV |
| | | DEHFSRALSQPSSYSPSCTSSKAPRSSGPWRDCSF |
| 429 | HXA11_HUMAN | DKAGGSSGQRTRKKRCPYTKYQIRELEREFFFSVYINKEKRLQ |
| | | LSRMLNLTDRQVKIWFQNRRMKEKKINRDRLQYYSAN |
| 430 | PDLI4_HUMAN | GAPLSGLQGLPECTRCGHGIVGTIVKARDKLYHPECFMCSDC |
| | | GLNLKQRGYFFLDERLYCESHAKARVKPPEGYDVVAVY |
| 431 | ASCL2_HUMAN | RRPATAETGGGAAAVARRNERERNRVKLVNLGFQALRQHVP |
| | | HGGASKKLSKVETLRSAVEYIRALQRLLAEHDAVRNALA |
| 432 | CDX4_HUMAN | TVQVTGKTRTKEKYRVVYTDHQRLELEKEFHCNRYITIQRKS |
| | | ELAVNLGLSERQVKIWFQNRRAKERKMIKKKISQFENS |
| 433 | ZN860_HUMAN | EEAAQKRKEKEPGMALPQGHLTFRDVAIEFSLEEWKCLDPTQ |
| | | RALYRAMMLENYRNLHSVDISSKCMMKKFSSTAQGNTE |
| 434 | LMBL4_HUMAN | DIRASQVARWTVDEVAEFVQSLLGCEEHAKCFKKEQIDGKAF |
| | | LLLTQTDIVKVMKIKLGPALKIYNSILMFRHSQELPEE |
| 435 | PDIP3_HUMAN | LSPLEGTKMTVNNLHPRVTEEDIVELFCVCGALKRARLVHPG |
| | | VAEVVFVKKDDAITAYKKYNNRCLDGQPMKCNLHMNGN |
| 436 | NKX25_HUMAN | DNAERPRARRRRKPRVLFSQAQVYELERRFKQQRYLSAPERD |
| | | QLASVLKLTSTQVKIWFQNRRYKCKRQRQDQTLELVGL |
| 437 | CEBPB_HUMAN | SQVKSKAKKTVDKHSDEYKIRRERNNIAVRKSRDKAKMRNL |
| | | ETQHKVLELTAENERLQKKVEQLSRELSTLRNLFKQLPE |
| 438 | ISL1_HUMAN | KRDYIRLYGIKCAKCSIGFSKNDFVMRARSKVYHIECFRCVAC |
| | | SRQLIPGDEFALREDGLFCRADHDVVERASLGAGDPL |
| 439 | CDX2_HUMAN | SLGSQVKTRTKDKYRVVYTDHQRLELEKEFHYSRYITIRRKAE |
| | | LAATLGLSERQVKIWFQNRRAKERKINKKKLQQQQQQ |
| 440 | PROP1_HUMAN | QGGQRGRPHSRRRHRTTFSPVQLEQLESAFGRNQYPDIWARE |
| | | SLARDTGLSEARIQVWFQNRRAKQRKQERSLLQPLAHL |
| 441 | SIN3B_HUMAN | DALTYLDQVKIRFGSDPATYNGFLEIMKEFKSQSIDTPGVIRRV |
| | | SQLFHEHPDLIVGFNAFLPLGYRIDIPKNGKLNIQS |
| 442 | SMBT1_HUMAN | RLHLDSNPLKWSVADVVRFIRSTDCAPLARIFLDQEIDGQALL |
| | | LLTLPTVQECMDLKLGPAIKLCHHIERIKFAFYEQFA |
| 443 | HXC11_HUMAN | AKGAAPNAPRTRKKRCPYSKFQIRELEREFFFNVYINKEKRLQ |
| | | LSRMLNLTDRQVKIWFQNRRMKEKKLSRDRLQYFSGN |
| 444 | HXC10_HUMAN | TTGNWLTAKSGRKKRCPYTKHQTLELEKEFLFNMYLTRERRL |
| | | EISKTINLTDRQVKIWFQNRRMKLKKMNRENRIRELTS |
| 445 | PRS6A_HUMAN | YLVSNVIELLDVDPNDQEEDGANIDLDSQRKGKCAVIKTSTRQ |
| | | TYFLPVIGLVDAEKLKPGDLVGVNKDSYLILETLPTE |
| 446 | VSX1_HUMAN | KASPTLGKRKKRRHRTVFTAHQLEELEKAFSEAHYPDVYARE |
| | | MLAVKTELPEDRIQVWFQNRRAKWRKREKRWGGSSVMA |
| 447 | NKX23_HUMAN | EESERPKPRSRRKPRVLFSQAQVFELERRFKQQRYLSAPEREH |
| | | LASSLKLTSTQVKIWFQNRRYKCKRQRQDKSLELGAH |
| 448 | MTG16_HUMAN | VVPGSRQEEVIDHKLTEREWAEEWKHLNNLLNCIMDMVEKT |
| | | RRSLTVLRRCQEADREELNHWARRYSDAEDTKKGPAPAA |
| 449 | HMX3_HUMAN | ESPEKKPACRKKKTRTVFSRSQVFQLESTFDMKRYLSSSERAG |
| | | LAASLHLTETQVKIWFQNRRNKWKRQLAAELEAANLS |
| 450 | HMX1_HUMAN | RGGVGVGGGRKKKTRTVFSRSQVFQLESTFDLKRYLSSAERA |
| | | GLAASLQLTETQVKIWFQNRRNKWKRQLAAELEAASLS |
| 451 | KIF22_HUMAN | ELLAHGRQKILDLLNEGSARDLRSLQRIGPKKAQLIVGWRELH |
| | | GPFSQVEDLERVEGITGKQMESFLKANILGLAAGQRC |
| 452 | CSTF2_HUMAN | ESPYGETISPEDAPESISKAVASLPPEQMFELMKQMKLCVQNS |
| | | PQEARNMLLQNPQLAYALLQAQVVMRIVDPEIALKIL |
| 453 | CEBPE_HUMAN | AGPLHKGKKAVNKDSLEYRLRRERNNIAVRKSRDKAKRRILE |
| | | TQQKVLEYMAENERLRSRVEQLTQELDTLRNLFRQIPE |
| 454 | DLX2_HUMAN | IRIVNGKPKKVRKPRTIYSSFQLAALQRRFQKTQYLALPERAE |
| | | LAASLGLTQTQVKIWFQNRRSKFKKMWKSGEIPSEQH |
| 455 | ZMYM3_HUMAN | TVYQFCSPSCWTKFQRTSPEGGIHLSCHYCHSLFSGKPEVLDW |
| | | QDQVFQFCCRDCCEDFKRLRGVVSQCEHCRQEKLLHE |
| 456 | PPARG_HUMAN | TMVDTEMPFWPTNFGISSVDLSVMEDHSHSFDIKPFTTVDFSSI |
| | | STPHYEDIPFTRTDPVVADYKYDLKLQEYQSAIKVE |
| 457 | PRIC1_HUMAN | GRHHAELLKPRCSACDEIIFADECTEAEGRHWHMKHFCCLEC |
| | | ETVLGGQRYIMKDGRPFCCGCFESLYAEYCETCGEHIG |
| 458 | UNC4_HUMAN | DPDKESPGCKRRRTRTNFTGWQLEELEKAFNESHYPDVFMRE |
| | | ALALRLDLVESRVQVWFQNRRAKWRKKENTKKGPGRPA |
| 459 | BARX2_HUMAN | TEQPTPRQKKPRRSRTIFTELQLMGLEKKFQKQKYLSTPDRLD |
| | | LAQSLGLTQLQVKTWYQNRRMKWKKMVLKGGQEAPTK |
| 460 | ALX3_HUMAN | SMELAKNKSKKRRNRTTFSTFQLEELEKVFQKTHYPDVYARE |
| | | QLALRTDLTEARVQVWFQNRRAKWRKRERYGKIQEGRN |
| 461 | TCF15_HUMAN | GGGGGAGPVVVVRQRQAANARERDRTQSVNTAFTALRTLIPT |
| | | EPVDRKLSKIETVRLASSYIAHLANVLLLGDSADDGQP |
| 462 | TERA_HUMAN | IDDTVEGITGNLFEVYLKPYFLEAYRPIRKGDIFLVRGGMRAV |
| | | EFKVVETDPSPYCIVAPDTVIHCEGEPIKREDEEESL |
| 463 | VSX2_HUMAN | SALNQTKKRKKRRHRTIFTSYQLEELEKAFNEAHYPDVYARE |
| | | MLAMKTELPEDRIQVWFQNRRAKWRKREKCWGRSSVMA |
| 464 | HXD12_HUMAN | DGLPWGAAPGRARKKRKPYTKQQIAELENEFLVNEFINRQKR |
| | | KELSNRLNLSDQQVKIWFQNRRMKKKRVVLREQALALY |
| 465 | CDX1HUMAN | GGGGSGKTRTKDKYRVVYTDHQRLELEKEFHYSRYITIRRKS |
| | | ELAANLGLTERQVKIWFQNRRAKERKVNKKKQQQQQPP |
| 466 | TCF23_HUMAN | TRAGGLALGRSEASPENAARERSRVRTLRQAFLALQAALPAV |
| | | PPDTKLSKLDVLVLAASYIAHLTRTLGHELPGPAWPPF |
| 467 | ALX1_HUMAN | KCDSNVSSSKKRRHRTTFTSLQLEELEKVFQKTHYPDVYVRE |
| | | QLALRTELTEARVQVWFQNRRAKWRKRERYGQIQQAKS |
| 468 | HXA10_HUMAN | NAANWLTAKSGRKKRCPYTKHQTLELEKEFLFNMYLTRERR |
| | | LEISRSVHLTDRQVKIWFQNRRMKLKKMNRENRIRELTA |
| 469 | RX_HUMAN | LSEEEQPKKKHRRNRTTFTTYQLHELERAFEKSHYPDVYSREE |
| | | LAGKVNLPEVRVQVWFQNRRAKWRRQEKLEVSSMKLQ |
| 470 | CXXC5_HUMAN | HMAGLAEYPMQGELASAISSGKKKRKRCGMCAPCRRRINCE |
| | | QCSSCRNRKTGHQICKFRKCEELKKKPSAALEKVMLPTG |
| 471 | SCML1_HUMAN | SITKHPSTWSVEAVVLFLKQTDPLALCPLVDLFRSHEIDGKAL |
| | | LLLTSDVLLKHLGVKLGTAVKLCYYIDRLKQGKCFEN |
| 472 | NFIL3_HUMAN | ACRRKREFIPDEKKDAMYWEKRRKNNEAAKRSREKRRLNDL |
| | | VLENKLIALGEENATLKAELLSLKLKFGLISSTAYAQEI |
| 473 | DLX6_HUMAN | EIRFNGKGKKIRKPRTIYSSLQLQALNHRFQQTQYLALPERAE |
| | | LAASLGLTQTQVKIWFQNKRSKFKKLLKQGSNPHESD |
| 474 | MTG8_HUMAN | GLHGTRQEEMIDHRLTDREWAEEWKHLDHLLNCIMDMVEKT |
| | | RRSLTVLRRCQEADREELNYWIRRYSDAEDLKKGGGSSS |
| 475 | CBX8_HUMAN | ELSAVGERVFAAEALLKRRIRKGRMEYLVKWKGWSQKYST |
| | | WEPEENILDARLLAAFEEREREMELYGPKKRGPKPKTFLL |
| 476 | CEBPD_HUMAN | AREKSAGKRGPDRGSPEYRQRRERNNIAVRKSRDKAKRRNQE |
| | | MQQKLVELSAENEKLHQRVEQLTRDLAGLRQFFKQLPS |
| 477 | SEC13_HUMAN | SGGCDNLIKLWKEEEDGQWKEEQKLEAHSDWVRDVAWAPSI |
| | | GLPTSTIASCSQDGRVFIWTCDDASSNTWSPKLLHKEND |
| 478 | FIP1_HUMAN | VKGVDLDAPGSINGVPLLEVDLDSFEDKPWRKPGADLSDYFN |
| | | YGFNEDTWKAYCEKQKRIRMGLEVIPVTSTTNKITAED |
| 479 | ALX4_HUMAN | KADSESNKGKKRRNRTTFTSYQLEELEKVFQKTHYPDVYARE |
| | | QLAMRTDLTEARVQVWFQNRRAKWRKRERFGQMQQVRT |
| 480 | LHX3_HUMAN | TAKQREAEATAKRPRTTITAKQLETLKSAYNTSPKPARHVRE |
| | | QLSSETGLDMRVVQVWFQNRRAKEKRLKKDAGRQRWGQ |
| 481 | PRIC2_HUMAN | GRHHAECLKPRCAACDEIIFADECTEAEGRHWHMKHFCCFEC |
| | | ETVLGGQRYIMKEGRPYCCHCFESLYAEYCDTCAQHIG |
| 482 | MAGI3_HUMAN | IIGGDRPDEFLQVKNVLKDGPAAQDGKIAPGDVIVDINGNCVL |
| | | GHTHADVVQMFQLVPVNQYVNLTLCRGYPLPDDSEDP |
| 483 | NELL1_HUMAN | CCPECDTRVTSQCLDQNGHKLYRSGDNWTHSCQQCRCLEGE |
| | | VDCWPLTCPNLSCEYTAILEGECCPRCVSDPCLADNITY |
| 484 | PRRX1_HUMAN | LNSEEKKKRKQRRNRTTFNSSQLQALERVFERTHYPDAFVRE |
| | | DLARRVNLTEARVQVWFQNRRAKFRRNERAMLANKNAS |
| 485 | MTG8R_HUMAN | GLNGGYQDELVDHRLTEREWADEWKHLDHALNCIMEMVEK |
| | | TRRSMAVLRRCQESDREELNYWKRRYNENTELRKTGTELV |
| 486 | RAX2_HUMAN | GPGEEAPKKKHRRNRTTFTTYQLHQLERAFEASHYPDVYSRE |
| | | ELAAKVHLPEVRVQVWFQNRRAKWRRQERLESGSGAVA |
| 487 | DLX3_HUMAN | VRMVNGKPKKVRKPRTIYSSYQLAALQRRFQKAQYLALPER |
| | | AELAAQLGLTQTQVKIWFQNRRSKFKKLYKNGEVPLEHS |
| 488 | DLX1_HUMAN | EVRFNGKGKKIRKPRTIYSSLQLQALNRRFQQTQYLALPERAE |
| | | LAASLGLTQTQVKIWFQNKRSKFKKLMKQGGAALEGS |
| 489 | NKX26_HUMAN | GRSEQPKARQRRKPRVLFSQAQVLALERRFKQQRYLSAPERE |
| | | HLASALQLTSTQVKIWFQNRRYKCKRQRQDKSLELAGH |
| 490 | NAB1_HUMAN | LPRTLGELQLYRILQKANLLSYFDAFIQQGGDDVQQLCEAGEE |
| | | EFLEIMALVGMASKPLHVRRLQKALRDWVTNPGLFNQ |
| 491 | SAMD7_HUMAN | NLSLDEDIQKWTVDDVHSFIRSLPGCSDYAQVFKDHAIDGETL |
| | | PLLTEEHLRGTMGLKLGPALKIQSQVSQHVGSMFYKK |
| 492 | PITX3_HUMAN | SPEDGSLKKKQRRQRTHFTSQQLQELEATFQRNRYPDMSTRE |
| | | EIAVWTNLTEARVRVWFKNRRAKWRKRERSQQAELCKG |
| 493 | WDR5_HUMAN | SNLLVSASDDKTLKIWDVSSGKCLKTLKGHSNYVFCCNFNPQ |
| | | SNLIVSGSFDESVRIWDVKTGKCLKTLPAHSDPVSAVH |
| 494 | MEOX2_HUMAN | GNYKSEVNSKPRKERTAFTKEQIRELEAEFAHHNYLTRLRRYE |
| | | IAVNLDLTERQVKVWFQNRRMKWKRVKGGQQGAAARE |
| 495 | NAB2_HUMAN | LPRTLGELQLYRVLQRANLLSYYETFIQQGGDDVQQLCEAGE |
| | | EEFLEIMALVGMATKPLHVRRLQKALREWATNPGLFSQ |
| 496 | DHX8_HUMAN | PEEPTIGDIYNGKVTSIMQFGCFVQLEGLRKRWEGLVHISELR |
| | | REGRVANVADVVSKGQRVKVKVLSFTGTKTSLSMKDV |
| 497 | FOXA2_HUMAN | YAFNHPFSINNLMSSEQQHHHSHHHHQPHKMDLKAYEQVMH |
| | | YPGYGSPMPGSLAMGPVTNKTGLDASPLAADTSYYQGVY |
| 498 | CBX6_HUMAN | TAAAGPAPPTAPEPAGASSEPEAGDWRPEMSPCSNVVVTDVT |
| | | SNLLTVTIKEFCNPEDFEKVAAGVAGAAGGGGSIGASK |
| 499 | EMX2_HUMAN | FLLHNALARKPKRIRTAFSPSQLLRLEHAFEKNHYVVGAERK |
| | | QLAHSLSLTETQVKVWFQNRRTKFKRQKLEEEGSDSQQ |
| 500 | CPSF6_HUMAN | KRIALYIGNLTWWTTDEDLTEAVHSLGVNDILEIKFFENRANG |
| | | QSKGFALVGVGSEASSKKLMDLLPKRELHGQNPVVTP |
| 501 | HXC12_HUMAN | SGAPWYPINSRSRKKRKPYSKLQLAELEGEFLVNEFITRQRRR |
| | | ELSDRLNLSDQQVKIWFQNRRMKKKRLLLREQALSFF |
| 502 | KDM4B_HUMAN | SDNLYPESITSRDCVQLGPPSEGELVELRWTDGNLYKAKFISS |
| | | VTSHIYQVEFEDGSQLTVKRGDIFTLEEELPKRVRSR |
| 503 | LMBL3_HUMAN | GIPASKVSKWSTDEVSEFIQSLPGCEEHGKVFKDEQIDGEAFLL |
| | | MTQTDIVKIMSIKLGPALKIFNSILMFKAAEKNSHN |
| 504 | PHX2A_HUMAN | EPSGLHEKRKQRRIRTTFTSAQLKELERVFAETHYPDIYTREEL |
| | | ALKIDLTEARVQVWFQNRRAKFRKQERAASAKGAAG |
| 505 | EMX1_HUMAN | LLLHGPFARKPKRIRTAFSPSQLLRLERAFEKNHYVVGAERKQ |
| | | LAGSLSLSETQVKVWFQNRRTKYKRQKLEEEGPESEQ |
| 506 | NC2B_HUMAN | SSGNDDDLTIPRAAINKMIKETLPNVRVANDARELVVNCCTEF |
| | | IHLISSEANEICNKSEKKTISPEHVIQALESLGFGSY |
| 507 | DLX4_HUMAN | ERRPQAPAKKLRKPRTIYSSLQLQHLNQRFQHTQYLALPERAQ |
| | | LAAQLGLTQTQVKIWFQNKRSKYKKLLKQNSGGQEGD |
| 508 | SRY_HUMAN | NVQDRVKRPMNAFIVWSRDQRRKMALENPRMRNSEISKQLG |
| | | YQWKMLTEAEKWPFFQEAQKLQAMHREKYPNYKYRPRRK |
| 509 | ZN777_HUMAN | EITRLAVWAAVQAVERKLEAQAMRLLTLEGRTGTNEKKIAD |
| | | CEKTAVEFANHLESKWVVLGTLLQEYGLLQRRLENMENL |
| 510 | NELL1_HUMAN | CEKDIDECSEGIIECHNHSRCVNLPGWYHCECRSGFHDDGTYS |
| | | LSGESCIDIDECALRTHTCWNDSACINLAGGFDCLCP |
| 511 | ZN398_HUMAN | AAISLWTVVAAVQAIERKVEIHSRRLLHLEGRTGTAEKKLASC |
| | | EKTVTELGNQLEGKWAVLGTLLQEYGLLQRRLENLEN |
| 512 | GATA3_HUMAN | GQNRPLIKPKRRLSAARRAGTSCANCQTTTTTLWRRNANGDP |
| | | VCNACGLYYKLHNINRPLTMKKEGIQTRNRKMSSKSKK |
| 513 | BSH_HUMAN | HAELPGKHCRRRKARTVFSDSQLSGLEKRFEIQRYLSTPERVE |
| | | LATALSLSETQVKTWFQNRRMKHKKQLRKSQDEPKAP |
| 514 | SF3B4_HUMAN | QDATVYVGGLDEKVSEPLLWELFLQAGPVVNTHMPKDRVTG |
| | | QHQGYGFVEFLSEEDADYAIKIMNMIKLYGKPIRVNKAS |
| 515 | TEAD1_HUMAN | PIDNDAEGVWSPDIEQSFQEALAIYPPCGRRKIILSDEGKMYGR |
| | | NELIARYIKLRTGKTRTRKQVSSHIQVLARRKSRDF |
| 516 | TEAD3_HUMAN | GLDNDAEGVWSPDIEQSFQEALAIYPPCGRRKIILSDEGKMYG |
| | | RNELIARYIKLRTGKTRTRKQVSSHIQVLARKKVREY |
| 517 | RGAP1_HUMAN | DSVGTPQSNGGMRLHDFVSKTVIKPESCVPCGKRIKFGKLSLK |
| | | CRDCRVVSHPECRDRCPLPCIPTLIGTPVKIGEGMLA |
| 518 | PHF1_HUMAN | SAPHSMTASSSSVSSPSPGLPRRSAPPSPLCRSLSPGTGGGVRG |
| | | GVGYLSRGDPVRVLARRVRPDGSVQYLVEWGGGGIF |
| 519 | FOXA1_HUMAN | GDPHYSFNHPFSINNLMSSSEQQHKLDFKAYEQALQYSPYGST |
| | | LPASLPLGSASVTTRSPIEPSALEPAYYQGVYSRPVL |
| 520 | GATA2_HUMAN | GQNRPLIKPKRRLSAARRAGTCCANCQTTTTTLWRRNANGDP |
| | | VCNACGLYYKLHNVNRPLTMKKEGIQTRNRKMSNKSKK |
| 521 | FOXO3_HUMAN | DSLSGSSLYSTSANLPVMGHEKFPSDLDLDMFNGSLECDMESI |
| | | IRSELMDADGLDFNFDSLISTQNVVGLNVGNFTGAKQ |
| 522 | ZN212_HUMAN | TEISLWTVVAAIQAVEKKMESQAARLQSLEGRTGTAEKKLAD |
| | | CEKMAVEFGNQLEGKWAVLGTLLQEYGLLQRRLENVEN |
| 523 | IRX4_HUMAN | MDSGTRRKNATRETTSTLKAWLQEHRKNPYPTKGEKIMLAII |
| | | TKMTLTQVSTWFANARRRLKKENKMTWPPRNKCADEKR |
| 524 | ZBED6_HUMAN | NIEKQIYLPSTRAKTSIVWHFFHVDPQYTWRAICNLCEKSVSR |
| | | GKPGSHLGTSTLQRHLQARHSPHWTRANKFGVASGEE |
| 525 | LHX4_HUMAN | AKQNDDSEAGAKRPRTTITAKQLETLKNAYKNSPKPARHVRE |
| | | QLSSETGLDMRVVQVWFQNRRAKEKRLKKDAGRHRWGQ |
| 526 | SIN3A_HUMAN | DALSYLDQVKLQFGSQPQVYNDFLDIMKEFKSQSIDTPGVISR |
| | | VSQLFKGHPDLIMGFNTFLPPGYKIEVQTNDMVNVTT |
| 527 | RBBP7_HUMAN | DDHTVCLWDINAGPKEGKIVDAKAIFTGHSAVVEDVAWHLL |
| | | HESLFGSVADDQKLMIWDTRSNTTSKPSHLVDAHTAEVN |
| 528 | NKX61_HUMAN | GSILLDKDGKRKHTRPTFSGQQIFALEKTFEQTKYLAGPERAR |
| | | LAYSLGMTESQVKVWFQNRRTKWRKKHAAEMATAKKK |
| 529 | TRI68_HUMAN | DPTALVEAIVEEVACPICMTFLREPMSIDCGHSFCHSCLSGLW |
| | | EIPGESQNWGYTCPLCRAPVQPRNLRPNWQLANVVEK |
| 530 | R51A1_HUMAN | QSLPKKVSLSSDTTRKPLEIRSPSAESKKPKWVPPAASGGSRSS |
| | | SSPLVVVSVKSPNQSLRLGLSRLARVKPLHPNATST |
| 531 | MB3L1_HUMAN | AKSSQRKQRDCVNQCKSKPGLSTSIPLRMSSYTFKRPVTRITP |
| | | HPGNEVRYHQWEESLEKPQQVCWQRRLQGLQAYSSAG |
| 532 | DLX5_HUMAN | VRMVNGKPKKVRKPRTIYSSFQLAALQRRFQKTQYLALPERA |
| | | ELAASLGLTQTQVKIWFQNKRSKIKKIMKNGEMPPEHS |
| 533 | NOTC1_HUMAN | LQCNNHACGWDGGDCSLNFNDPWKNCTQSLQCWKYFSDGH |
| | | CDSQCNSAGCLFDGFDCQRAEGQCNPLYDQYCKDHFSDGH |
| 534 | TERF2_HUMAN | ETWVEEDELFQVQAAPDEDSTTNITKKQKWTVEESEWVKAG |
| | | VQKYGEGNWAAISKNYPFVNRTAVMIKDRWRTMKRLGMN |
| 535 | ZN282_HUMAN | AEISLWTVVAAIQAVERKVDAQASQLLNLEGRTGTAEKKLAD |
| | | CEKTAVEFGNHMESKWAVLGTLLQEYGLLQRRLENLEN |
| 536 | RGS12_HUMAN | LEKRTLFRLDLVPINRSVGLKAKPTKPVTEVLRPVVARYGLDL |
| | | SGLLVRLSGEKEPLDLGAPISSLDGQRVVLEEKDPSR |
| 537 | ZN840_HUMAN | PNCLSSSMQLPHGGGRHQELVRFRDVAVVFSPEEWDHLTPEQ |
| | | RNLYKDVMLDNCKYLASLGNWTYKAHVMSSLKQGKEPW |
| 538 | SPI2B_HUMAN | DDYKEGDLRIMPESSESPPTEREPGGVVDGLIGKHVEYTKEDG |
| | | SKRIGMVIHQVEAKPSVYFIKFDDDFHIYVYDLVKKS |
| 539 | PAX7_HUMAN | SEPDLPLKRKQRRSRTTFTAEQLEELEKAFERTHYPDIYTREEL |
| | | AQRTKLTEARVQVWFSNRRARWRKQAGANQLAAFNH |
| 540 | NKX62_HUMAN | AGGVLDKDGKKKHSRPTFSGQQIFALEKTFEQTKYLAGPERA |
| | | RLAYSLGMTESQVKVWFQNRRTKWRKRHAVEMASAKKK |
| 541 | ASXL2_HUMAN | DVMSFSVTVTTIPASQAMNPSSHGQTIPVQAFSEENSIEGTPSK |
| | | CYCRLKAMIMCKGCGAFCHDDCIGPSKLCVSCLVVR |
| 542 | FOX01_HUMAN | GGYSSVSSCNGYGRMGLLHQEKLPSDLDGMFIERLDCDMESII |
| | | RNDLMDGDTLDFNFDNVLPNQSFPHSVKTTTHSWVSG |
| 543 | GATA3_HUMAN | GGSPTGFGCKSRPKARSSTGRECVNCGATSTPLWRRDGTGHY |
| | | LCNACGLYHKMNGQNRPLIKPKRRLSAARRAGTSCANC |
| 544 | GATA1_HUMAN | GQNRPLIRPKKRLIVSKRAGTQCTNCQTTTTTLWRRNASGDP |
| | | VCNACGLYYKLHQVNRPLTMRKDGIQTRNRKASGKGKK |
| 545 | ZMYM5_HUMAN | PVALLRKQNFQPTAQQQLTKPAKITCANCKKPLQKGQTAYQR |
| | | KGSAHLFCSTTCLSSFSHKRTQNTRSIICKKDASTKKA |
| 546 | ZN783_HUMAN | TEITLWTVVAAIQALEKKVDSCLTRLLTLEGRTGTAEKKLAD |
| | | CEKTAVEFGNQLEGKWAVLGTLLQEYGLLQRRLENVEN |
| 547 | SPI2B_HUMAN | KKQRGRPSSQPRRNIVGCRISHGWKEGDEPITQWKGTVLDQV |
| | | PINPSLYLVKYDGIDCVYGLELHRDERVLSLKILSDRV |
| 548 | LRP1_HUMAN | WTCDLDDDCGDRSDESASCAYPTCFPLTQFTCNNGRCININW |
| | | RCDNDNDCGDNSDEAGCSHSCSSTQFKCNSGRCIPEHW |
| 549 | MIXL1_HUMAN | PKGAAAPSASQRRKRTSFSAEQLQLLELVFRRTRYPDIHLRER |
| | | LAALTLLPESRIQVWFQNRRAKSRRQSGKSFQPLARP |
| 550 | SGT1_HUMAN | KIKYDWYQTESQVVITLMIKNVQKNDVNVEFSEKELSALVKL |
| | | PSGEDYNLKLELLHPIIPEQSTFKVLSTKIEIKLKKPE |
| 551 | LMCD1_HUMAN | DPSKEVEYVCELCKGAAPPDSPVVYSDRAGYNKQWHPTCFV |
| | | CAKCSEPLVDLIYFWKDGAPWCGRHYCESLRPRCSGCDE |
| 552 | CEBPA_HUMAN | GSGAGKAKKSVDKNSNEYRVRRERNNIAVRKSRDKAKQRNV |
| | | ETQQKVLELTSDNDRLRKRVEQLSRELDTLRGIFRQLPE |
| 553 | GATA2_HUMAN | GPASSFTPKQRSKARSCSEGRECVNCGATATPLWRRDGTGHY |
| | | LCNACGLYHKMNGQNRPLIKPKRRLSAARRAGTCCANC |
| 554 | SOX14_HUMAN | KPSDHIKRPMNAFMVWSRGQRRKMAQENPKMHNSEISKRLG |
| | | AEWKLLSEAEKRPYIDEAKRLRAQHMKEHPDYKYRPRRK |
| 555 | WTIP_HUMAN | LYSGFQQTADKCSVCGHLIMEMILQALGKSYHPGCFRCSVCN |
| | | ECLDGVPFTVDVENNIYCVRDYHTVFAPKCASCARPIL |
| 556 | PRP19_HUMAN | HPSQDLVFSASPDATIRIWSVPNASCVQVVRAHESAVTGLSLH |
| | | ATGDYLLSSSDDQYWAFSDIQTGRVLTKVTDETSGCS |
| 557 | CBX6_HUMAN | ELSAVGERVFAAESIIKRRIRKGRIEYLVKWKGWAIKYSTWEP |
| | | EENILDSRLIAAFEQKERERELYGPKKRGPKPKTFLL |
| 558 | NKX11_HUMAN | RTGSDSKSGKPRRARTAFTYEQLVALENKFKATRYLSVCERL |
| | | NLALSLSLTETQVKIWFQNRRTKWKKQNPGADTSAPTG |
| 559 | RBBP4_HUMAN | VWDLSKIGEEQSPEDAEDGPPELLFIHGGHTAKISDFSWNPNE |
| | | PWVICSVSEDNIMQVWQMAENIYNDEDPEGSVDPEGQ |
| 560 | DMRT2_HUMAN | ERCTPAGGGAEPRKLSRTPKCARCRNHGVVSCLKGHKRFCR |
| | | WRDCQCANCLLVVERQRVMAAQVALRRQQATEDKKGLSG |
| 561 | SMCA2_HUMAN | SQPGALIPGDPQAMSQPNRGPSPFSPVQLHQLRAQILAYKMLA |
| | | RGQPLPETLQLAVQGKRTLPGLQQQQQQQQQQQQQQQ |
| 562 | ZNF10 | MDAKSLTAWSRTLVTFKDVFVDFTREEWKLLDTAQQIVYRN |
| | | VMLENYKNLVSLGYQLTKPDVILRLEKGEEPWLVEREIHQET |
| | | HPDSETAFEIKSSVSSRSIFKDKQSCDIKMEGMARNDLWYLSL |
| | | EEVWKCRDQLDKYQENPERHLRQVAFTQKKVLTQERVSESG |
| | | KYGGNCLLPAQLVLREYFHKRDSHTKSLKHDLVLNGHQDSC |
| | | ASNSNECGQTFCQNIHLIQFARTHTGDKSYKCPDNDNSLTHGS |
| | | SLGISKGIHREKPYECKECGKFFSWRSNLTRHQLIHTGEKPYEC |
| | | KECGKSFSRSSHLIGHQKTHTGEEPYECKECGKSFSWFSHLVT |
| | | HQRTHTGDKLYTCNQCGKSFVHSSRLIRHQRTHTGEKPYECP |
| | | ECGKSFRQSTHLILHQRTHVRVRPYECNECGKSYSQRSHLVV |
| | | HHRIHTGLKPFECKDCGKCFSRSSHLYSHQRTHTGEKPYECHD |
| | | CGKSFSQSSALIVHQRIHTGEKPYECCQCGKAFIRKNDLIKHQ |
| | | RIHVGEETYKCNQCGIIFSQNSPFIVHQIAHTGEQFLTCNQCGT |
| | | ALVNTSNLIGYQTNHIRENAY |
| 563 | EED_HUMAN | MSEREVSTAPAGTDMPAAKKQKLSSDENSNPDLSGDENDDA |
| | | VSIESGTNTERPDTPTNTPNAPGRKSWGKGKWKSKKCKYSFK |
| | | CVNSLKEDHNQPLFGVQFNWHSKEGDPLVFATVGSNRVTLY |
| | | ECHSQGEIRLLQSYVDADADENFYTCAWTYDSNTSHPLLAVA |
| | | GSRGIIRIINPITMQCIKHYVGHGNAINELKFHPRDPNLLLSVSK |
| | | DHALRLWNIQTDTLVAIFGGVEGHRDEVLSADYDLLGEKIMS |
| | | CGMDHSLKLWRINSKRMMNAIKESYDYNPNKTNRPFISQKIH |
| | | FPDFSTRDIHRNYVDCVRWLGDLILSKSCENAIVCWKPGKME |
| | | DDIDKIKPSESNVTILGRFDYSQCDIWYMRFSMDFWQKMLAL |
| | | GNQVGKLYVWDLEVEDPHKAKCTTLTHHKCGAAIRQTSFSR |
| | | DSSILIAVCDDASIWRWDRLR |
| 564 | RCOR1_HUMAN | MPAMVEKGPEVSGKRRGRNNAAASASAAAASAAASAACASP |
| | | AATAASGAAASSASAAAASAAAAPNNGQNKSLAAAAPNGNS |
| | | SSNSWEEGSSGSSSDEEHGGGGMRVGPQYQAVVPDFDPAKL |
| | | ARRSQERDNLGMLVWSPNQNLSEAKLDEYIAIAKEKHGYNM |
| | | EQALGMLFWHKHNIEKSLADLPNFTPFPDEWTVEDKVLFEQA |
| | | FSFHGKTFHRIQQMLPDKSIASLVKFYYSWKKTRTKTSVMDR |
| | | HARKQKREREESEDELEEANGNNPIDIEVDQNKESKKEVPPTE |
| | | TVPQVKKEKHSTQAKNRAKRKPPKGMFLSQEDVEAVSANAT |
| | | AATTVLRQLDMELVSVKRQIQNIKQTNSALKEKLDGGIEPYRL |
| | | PEVIQKCNARWTTEEQLLAVQAIRKYGRDFQAISDVIGNKSVV |
| | | QVKNFFVNYRRRFNIDEVLQEWEAEHGKEETNGPSNQKPVKS |
| | | PDNSIKMPEEEDEAPVLDVRYASAS |
| 565 | KOX1/ZNF10 | TGRTLVTFKDVFVDFTREEWKLLDTAQQIVYRNVMLENYKN |
| | KRAB 1 | LVSLGYQLTKPDVILRLEKGEEPLEINLWITKFVKD |
| 566 | KOX1/ZNF10 | MYPYDVPDYASPKKKRKVGGGASMDAKSLTAWSRTLVTFK |
| | KRAB 2 | DVFVDFTREEWKLLDTAQQIVYRNVMLENYKNLVSLGYQLT |
| | | KPDVILRLEKGEEPWLVEREIHQETHPDSETAFEIKSSV |
| 567 | KOX1/ZNF10 | ALSPQHSAVTQGSIIKNKEGMDAKSLTAWSRTLVTFKDVFVD |
| | KRAB 3 | FTREEWKLLDTAQQIVYRNVMLENYKNLVSLGYQLTKPDVIL |
| | | RLEKGEEPWLVEREIHQETHPDSETAFEIKSSV |
| 568 | KOX1/ZNF10 (aa | RTLVTFKDVFVDFTREEWKLLDTAQQIVYRNVMLENYKNLV |
| | 11-72) | SLGYQLTKPDVILRLEKGEEP |
| 569 | KOX1/ZNF10 (aa | RTLVTFKDVFVDFTREEWKLLDTAQQIVYRNVMLENYKNLV |
| | 11-108) | SLGYQLTKPDVILRLEKGEEPWLVEREIHQETHPDSETAFEIKS |
| | | SVSSRSIFKDKQS |
| 570 | KOX1/ZNF10 | RTLVTFKDVAVDFTQEEWQQLDPAQKIVYRDVMLENYSNLV |
| | variant | SVGYQLTKPDVILRLEQKGEEPWLVEEEIHQETHPDSETAFEIK |
| | | SSVSSRSIFKDKQS |
| 571 | KOX1 KRAB- | RTLVTFKDVFVDFTREEWKLLDTAQQIVYRNVMLENYKNLV |
| | ZIM3 chimera | SLGYQLTKPDVILRLEKGEEPWLEEEEVLGSGRAEKNGDIGGQ |
| | | IWKPKDVKESL |
| 572 | ZIM3-KOX1 | MNNSQGRVTFEDVTVNFTQGEWQRLNPEQRNLYRDVMLEN |
| | KRAB chimera | YSNLVSVGQGETTKPDVILRLEQGKEPWLVEREIHQETHPDSE |
| | | TAFEIKSSVSSRSIFKDKQS |
| 573 | human DNMT1 | MPARTAPARVPTLAVPAISLPDDVRRRLKDLERDSLTEKECVK |
| | | EKLNLLHEFLQTEIKNQLCDLETKLRKEELSEEGYLAKVKSLL |
| | | NKDLSLENGAHAYNREVNGRLENGNQARSEARRVGMADAN |
| | | SPPKPLSKPRTPRRSKSDGEAKPEPSPSPRITRKSTRQTTITSHF |
| | | AKGPAKRKPQEESERAKSDESIKEEDKDQDEKRRRVTSRERV |
| | | ARPLPAEEPERAKSGTRTEKEEERDEKEEKRLRSQTKEPTPKQ |
| | | KLKEEPDREARAGVQADEDEDGDEKDEKKHRSQPKDLAAKR |
| | | RPEEKEPEKVNPQISDEKDEDEKEEKRRKTTPKEPTEKKMARA |
| | | KTVMNSKTHPPKCIQCGQYLDDPLKYGQHPPDAVDEPQMLT |
| | | NEKLSIFDANESGFESYEALPQHKLTCFSVYCKHGHLCPIDTG |
| | | LIEKNIELFFSGSAKPIYDDDPSLEGGVNGKNLGPINEWWITGF |
| | | DGGEKALIGFSTSFAEYILMDPSPEYAPIFGLMQEKIYISKIVVE |
| | | FLQSNSDSTYEDLINKIETTVPPSGLNLNRFTEDSLLRHAQFVV |
| | | EQVESYDEAGDSDEQPIFLTPCMRDLIKLAGVTLGQRRAQAR |
| | | RQTIRHSTREKDRGPTKATTTKLVYQIFDTFFAEQIEKDDRED |
| | | KENAFKRRRCGVCEVCQQPECGKCKACKDMVKFGGSGRSKQ |
| | | ACQERRCPNMAMKEADDDEEVDDNIPEMPSPKKMHQGKKK |
| | | KQNKNRISWVGEAVKTDGKKSYYKKVCIDAETLEVGDCVSVI |
| | | PDDSSKPLYLARVTALWEDSSNGQMFHAHWFCAGTDTVLGA |
| | | TSDPLELFLVDECEDMQLSYIHSKVKVIYKAPSENWAMEGGM |
| | | DPESLLEGDDGKTYFYQLWYDQDYARFESPPKTQPTEDNKFK |
| | | FCVSCARLAEMRQKEIPRVLEQLEDLDSRVLYYSATKNGILYR |
| | | VGDGVYLPPEAFTFNIKLSSPVKRPRKEPVDEDLYPEHYRKYS |
| | | DYIKGSNLDAPEPYRIGRIKEIFCPKKSNGRPNETDIKIRVNKFY |
| | | RPENTHKSTPASYHADINLLYWSDEEAVVDFKAVQGRCTVEY |
| | | GEDLPECVQVYSMGGPNRFYFLEAYNAKSKSFEDPPNHARSP |
| | | GNKGKGKGKGKGKPKSQACEPSEPEIEIKLPKLRTLDVFSGCG |
| | | GLSEGFHQAGISDTLWAIEMWDPAAQAFRLNNPGSTVFTEDC |
| | | NILLKLVMAGETTNSRGQRLPQKGDVEMLCGGPPCQGFSGM |
| | | NRFNSRTYSKFKNSLVVSFLSYCDYYRPRFFLLENVRNFVSFK |
| | | RSMVLKLTLRCLVRMGYQCTFGVLQAGQYGVAQTRRRAIIL |
| | | AAAPGEKLPLFPEPLHVFAPRACQLSVVVDDKKFVSNITRLSS |
| | | GPFRTITVRDTMSDLPEVRNGASALEISYNGEPQSWFQRQLRG |
| | | AQYQPILRDHICKDMSALVAARMRHIPLAPGSDWRDLPNIEV |
| | | RLSDGTMARKLRYTHHDRKNGRSSSGALRGVCSCVEAGKAC |
| | | DPAARQFNTLIPWCLPHTGNRHNHWAGLYGRLEWDGFFSTT |
| | | VTNPEPMGKQGRVLHPEQHRVVSVRECARSQGFPDTYRLFGN |
| | | ILDKHRQVGNAVPPPLAKAIGLEIKLCMLAKARESASAKIKEE |
| | | EAAKD |
| 574 | human DNMT3A | MPAMPSSGPGDTSSSAAEREEDRKDGEEQEEPRGKEERQEPST |
| | | TARKVGRPGRKRKHPPVESGDTPKDPAVISKSPSMAQDSGAS |
| | | ELLPNGDLEKRSEPQPEEGSPAGGQKGGAPAEGEGAAETLPE |
| | | ASRAVENGCCTPKEGRGAPAEAGKEQKETNIESMKMEGSRG |
| | | RLRGGLGWESSLRQRPMPRLTFQAGDPYYISKRKRDEWLAR |
| | | WKREAEKKAKVIAGMNAVEENQGPGESQKVEEASPPAVQQP |
| | | TDPASPTVATTPEPVGSDAGDKNATKAGDDEPEYEDGRGFGI |
| | | GELVWGKLRGFSWWPGRIVSWWMTGRSRAAEGTRWVMWF |
| | | GDGKFSVVCVEKLMPLSSFCSAFHQATYNKQPMYRKAIYEVL |
| | | QVASSRAGKLFPVCHDSDESDTAKAVEVQNKPMIEWALGGF |
| | | QPSGPKGLEPPEEEKNPYKEVYTDMWVEPEAAAYAPPPPAKK |
| | | PRKSTAEKPKVKEIIDERTRERLVYEVRQKCRNIEDICISCGSL |
| | | NVTLEHPLFVGGMCQNCKNCFLECAYQYDDDGYQSYCTICC |
| | | GGREVLMCGNNNCCRCFCVECVDLLVGPGAAQAAIKEDPWN |
| | | CYMCGHKGTYGLLRRREDWPSRLQMFFANNHDQEFDPPKVY |
| | | PPVPAEKRKPIRVLSLFDGIATGLLVLKDLGIQVDRYIASEVCE |
| | | DSITVGMVRHQGKIMYVGDVRSVTQKHIQEWGPFDLVIGGSP |
| | | CNDLSIVNPARKGLYEGTGRLFFEFYRLLHDARPKEGDDRPFF |
| | | WLFENVVAMGVSDKRDISRFLESNPVMIDAKEVSAAHRARYF |
| | | WGNLPGMNRPLASTVNDKLELQECLEHGRIAKFSKVRTITTRS |
| | | NSIKQGKDQHFPVFMNEKEDILWCTEMERVFGFPVHYTDVSN |
| | | MSRLARQRLLGRSWSVPVIRHLFAPLKEYFACV |
| 575 | human DNMT3A | NHDQEFDPPKVYPPVPAEKRKPIRVLSLFDGIATGLLVLKDLGI |
| | catalytic domain | QVDRYIASEVCEDSITVGMVRHQGKIMYVGDVRSVTQKHIQE |
| | | WGPFDLVIGGSPCNDLSIVNPARKGLYEGTGRLFFEFYRLLHD |
| | | ARPKEGDDRPFFWLFENVVAMGVSDKRDISRFLESNPVMIDA |
| | | KEVSAAHRARYFWGNLPGMNRPLASTVNDKLELQECLEHGR |
| | | IAKFSKVRTITTRSNSIKQGKDQHFPVFMNEKEDILWCTEMER |
| | | VFGFPVHYTDVSNMSRLARQRLLGRSWSVPVIRHLFAPLKEY |
| | | FACV |
| 576 | human DNMT3B | MKGDTRHLNGEEDAGGREDSILVNGACSDQSSDSPPILEAIRT |
| | | PEIRGRRSSSRLSKREVSSLLSYTQDLTGDGDGEDGDGSDTPV |
| | | MPKLFRETRTRSESPAVRTRNNNSVSSRERHRPSPRSTRGRQG |
| | | RNHVDESPVEFPATRSLRRRATASAGTPWPSPPSSYLTIDLTDD |
| | | TEDTHGTPQSSSTPYARLAQDSQQGGMESPQVEADSGDGDSS |
| | | EYQDGKEFGIGDLVWGKIKGFSWWPAMVVSWKATSKRQAM |
| | | SGMRWVQWFGDGKFSEVSADKLVALGLFSQHFNLATFNKLV |
| | | SYRKAMYHALEKARVRAGKTFPSSPGDSLEDQLKPMLEWAH |
| | | GGFKPTGIEGLKPNNTQPVVNKSKVRRAGSRKLESRKYENKT |
| | | RRRTADDSATSDYCPAPKRLKTNCYNNGKDRGDEDQSREQM |
| | | ASDVANNKSSLEDGCLSCGRKNPVSFHPLFEGGLCQTCRDRF |
| | | LELFYMYDDDGYQSYCTVCCEGRELLLCSNTSCCRCFCVECL |
| | | EVLVGTGTAAEAKLQEPWSCYMCLPQRCHGVLRRRKDWNV |
| | | RLQAFFTSDTGLEYEAPKLYPAIPAARRRPIRVLSLFDGIATGY |
| | | LVLKELGIKVGKYVASEVCEESIAVGTVKHEGNIKYVNDVRNI |
| | | TKKNIEEWGPFDLVIGGSPCNDLSNVNPARKGLYEGTGRLFFE |
| | | FYHLLNYSRPKEGDDRPFFWMFENVVAMKVGDKRDISRFLEC |
| | | NPVMIDAIKVSAAHRARYFWGNLPGMNRPVIASKNDKLELQ |
| | | DCLEYNRIAKLKKVQTITTKSNSIKQGKNQLFPVVMNGKEDV |
| | | LWCTELERIFGFPVHYTDVSNMGRGARQKLLGRSWSVPVIRH |
| | | LFAPLKDYFACE |
| 577 | mouse DNMT3C | MRGGSRHLSNEEDVSGCEDCIIISGTCSDQSSDPKTVPLTQVLE |
| | | AVCTVENRGCRTSSQPSKRKASSLISYVQDLTGDGDEDRDGE |
| | | VGGSSGSGTPVMPQLFCETRIPSKTPAPLSWQANTSASTPWLS |
| | | PASPYPIIDLTDEDVIPQSISTPSVDWSQDSHQEGMDTTQVDAE |
| | | SRDGGNIEYQVSADKLLLSQSCILAAFYKLVPYRESIYRTLEK |
| | | ARVRAGKACPSSPGESLEDQLKPMLEWAHGGFKPTGIEGLKP |
| | | NKKQPENKSRRRTTNDPAASESSPPKRLKTNSYGGKDRGEDE |
| | | ESREQMASDVTNNKGNLEDHCLSCGRKDPVSFHPLFEGGLCQ |
| | | SCRDRFLELFYMYDEDGYQSYCTVCCEGRELLLCSNTSCCRC |
| | | FCVECLEVLVGAGTAEDVKLQEPWSCYMCLPQRCHGVLRRR |
| | | KDWNMRLQDFFTTDPDLEEFEPPKLYPAIPAAKRRPIRVLSLF |
| | | DGIATGYLVLKELGIKVEKYIASEVCAESIAVGTVKHEGQIKY |
| | | VDDIRNITKEHIDEWGPFDLVIGGSPCNDLSCVNPVRKGLFEG |
| | | TGRLFFEFYRLLNYSCPEEEDDRPFFWMFENVVAMEVGDKRD |
| | | ISRFLECNPVMIDAIKVSAAHRARYFWGNLPGMNRPVMASKN |
| | | DKLELQDCLEFSRTAKLKKVQTITTKSNSIRQGKNQLFPVVMN |
| | | GKDDVLWCTELERIFGFPEHYTDVSNMGRGARQKLLGRSWS |
| | | VPVIRHLFAPLKDHFACE |
| 578 | human DNMT3L | MAAIPALDPEAEPSMDVILVGSSELSSSVSPGTGRDLIAYEVKA |
| | | NQRNIEDICICCGSLQVHTQHPLFEGGICAPCKDKFLDALFLYD |
| | | DDGYQSYCSICCSGETLLICGNPDCTRCYCFECVDSLVGPGTS |
| | | GKVHAMSNWVCYLCLPSSRSGLLQRRRKWRSQLKAFYDRES |
| | | ENPLEMFETVPVWRRQPVRVLSLFEDIKKELTSLGFLESGSDP |
| | | GQLKHVVDVTDTVRKDVEEWGPFDLVYGATPPLGHTCDRPP |
| | | SWYLFQFHRLLQYARPKPGSPRPFFWMFVDNLVLNKEDLDV |
| | | ASRFLEMEPVTIPDVHGGSLQNAVRVWSNIPAIRSSRHWALVS |
| | | EEELSLLAQNKQSSKLAAKWPTKLVKNCFLPLREYFKYFSTEL |
| | | TSSL |
| 579 | human DNMT3L | NPLEMFETVPVWRRQPVRVLSLFEDIKKELTSLGFLESGSDPG |
| | catalytic domain | QLKHVVDVTDTVRKDVEEWGPFDLVYGATPPLGHTCDRPPS |
| | | WYLFQFHRLLQYARPKPGSPRPFFWMFVDNLVLNKEDLDVA |
| | | SRFLEMEPVTIPDVHGGSLQNAVRVWSNIPAIRSRHWALVSEE |
| | | ELSLLAQNKQSSKLAAKWPTKLVKNCFLPLREYFKYFSTELTS |
| | | SL |
| 580 | mouse DNMT3L | MGSRETPSSCSKTLETLDLETSDSSSPDADSPLEEQWLKSSPAL |
| | | KEDSVDVVLEDCKEPLSPSSPPTGREMIRYEVKVNRRSIEDICL |
| | | CCGTLQVYTRHPLFEGGLCAPCKDKFLESLFLYDDDGHQSYC |
| | | TICCSGGTLFICESPDCTRCYCFECVDILVGPGTSERINAMACW |
| | | VCFLCLPFSRSGLLQRRKRWRHQLKAFHDQEGAGPMEIYKTV |
| | | SAWKRQPVRVLSLFRNIDKVLKSLGFLESGSGSGGGTLKYVE |
| | | DVTNVVRRDVEKWGPFDLVYGSTQPLGSSCDRCPGWYMFQF |
| | | HRILQYALPRQESQRPFFWIFMDNLLLTEDDQETTTRFLQTEA |
| | | VTLQDVRGRDYQNAMRVWSNIPGLKSKHAPLTPKEEEYLQA |
| | | QVRSRSKLDAPKVDLLVKNCLLPLREYFKYFSQNSLPL |
| 581 | mouse DNMT3L | GPMEIYKTVSAWKRQPVRVLSLFRNIDKVLKSLGFLESGSGSG |
| | catalytic domain | GGTLKYVEDVTNVVRRDVEKWGPFDLVYGSTQPLGSSCDRC |
| | | PGWYMFQFHRILQYALPRQESQRPFFWIFMDNLLLTEDDQET |
| | | TTRFLQTEAVTLQDVRGRDYQNAMRVWSNIPGLKSKHAPLTP |
| | | KEEEYLQAQVRSRSKLDAPKVDLLVKNCLLPLREYFKYFSQN |
| | | SLPL |
| 582 | Ailuropoda | MALSPTGTLSVETLDRSDPDPLDEGPWQATCEILLEPDAEHST |
| | melanoleuca | DVILVGSSELSAPASPGPRRDLLAYEVKVNQRDIEDVCICCGSL |
| | DNMT3L | RVHTQHPLFEGGMCAPCKDKFLDCLFLYDDDGYQSYCSICCA |
| | | GETLLICENPDCTRPSLMMKLRLFRECACLIFPSEGMLLQTVW |
| | | FWKMTVVWQPGLRHLPQENPLETYKTVPVWKREPVRVLSLF |
| | | GDIRRELMSLGFLESGSAPGRLKHLDDVTDVVRKDVEGWGPF |
| | | DLVYGSTPPIGHACDHPPVWYLLQFHRILQYARPRPGSQQPFF |
| | | WMFVDNLVLSQDDQTAATRFLEADPVTIQDVCGRAVRNTVH |
| | | VWSNIPAVRSRHSALALCEELSLLAQDRQRTKPPAQGPAQLV |
| | | KNCFLPLREYFKYFSTELTSSL |
| 583 | Ailuropoda | NPLETYKTVPVWKREPVRVLSLFGDIRRELMSLGFLESGSAPG |
| | melanoleuca | RLKHLDDVTDVVRKDVEGWGPFDLVYGSTPPIGHACDHPPV |
| | DNMT3L catalytic | WYLLQFHRILQYARPRPGSQQPFFWMFVDNLVLSQDDQTAA |
| | domain | TRFLEADPVTIQDVCGRAVRNTVHVWSNIPAVRSRHSALALC |
| | | EELSLLAQDRQRTKPPAQGPAQLVKNCFLPLREYFKYFSTELT |
| | | SSL |
| 584 | Carlito syrichta | MALSCRRTLPLESLHSSNSDLASQLDKEQWRPPCETHGIPVAA |
| | DNMT3L | APVLDLEAECSLDVILVGSSELSTSSSPRLGRDHIAYEVKVNQR |
| | | NIEDICLCCGSFLVHTQHPLFEGGMCAPCKDKFLDTLFLYDED |
| | | GYQSYCSICCSGETLLICENPDCTRCYCFECLDTLVSPGTSEKV |
| | | HAMSNWVCFLCLPFTRSGLLQRRRKWRGQLKAFYDRESESSL |
| | | EMYKTVPVWKREPVRVLSLFGDIKKELMSLGFVETGSDPGRL |
| | | RHLDDTTNIVRRNVEEWGPFHLLYGATPPLGHTCDRPPGWYL |
| | | FQFHRLLQYARPQPGSPQPFFWMFVDNVMLTREDRAIASRFL |
| | | ETEPVTIPDIHGRALQNAVCVWSNIPAVRSKHSALVSEEELSLL |
| | | AQDRQRAKLPTQGPTKLVKNCFLPLREYFKYFSTELTSFL |
| 585 | Carlito syrichta | SSLEMYKTVPVWKREPVRVLSLFGDIKKELMSLGFVETGSDP |
| | DNMT3L catalytic | GRLRHLDDTTNIVRRNVEEWGPFHLLYGATPPLGHTCDRPPG |
| | domain | WYLFQFHRLLQYARPQPGSPQPFFWMFVDNVMLTREDRAIAS |
| | | RFLETEPVTIPDIHGRALQNAVCVWSNIPAVRSKHSALVSEEEL |
| | | SLLAQDRQRAKLPTQGPTKLVKNCFLPLREYFKYFSTELTSFL |
| 586 | Meriones | MGSQETPSTRAKTPGTWNLESTDSSSPESLGHLEEQWANSSPD |
| | unguiculatus | LKDEHSKDVEPEDSKELISSASPPSGREIIRYEISVNQRNIEDICL |
| | DNMT3L | CCGTLQVYKQHPLFEGGICAPCKDKFLETFFLYDEDGHQSYCS |
| | | ICCSGGTLFICESPDCTRCYCFECVDILVGPGTSERINAMPCWV |
| | | CFLCLPFTRSGLLQRRRKWRHQLKAFFDEGGASPLEMYKTVS |
| | | AWKRKPMRVLSLFKNIDKELKNLGFLESGSGSEEERLKYLED |
| | | VTNVVRRDVEKWGPFDLVYGSTRPRGSSCDHCPAWYMFQFH |
| | | RILQYARPPSGSEQPFFWVFVDNLLMTEDDQITADRFLQMKA |
| | | VTLQDVRGRVLQNAVRVWSNIPGVKSKHMALTEKEEQSLEA |
| | | QAGTRTKLSAQKVDPLVKNCLLPLREYFKFFSQNSLPLDK |
| 587 | Meriones | SPLEMYKTVSAWKRKPMRVLSLFKNIDKELKNLGFLESGSGS |
| | unguiculatus | EEERLKYLEDVINVVRRDVEKWGPFDLVYGSTRPRGSSCDHC |
| | DNMT3L catalytic | PAWYMFQFHRILQYARPPSGSEQPFFWVFVDNLLMTEDDQIT |
| | domain | ADRFLQMKAVTLQDVRGRVLQNAVRVWSNIPGVKSKHMAL |
| | | TEKEEQSLEAQAGTRTKLSAQKVDPLVKNCLLPLREYFKFFSQ |
| | | NSLPLDK |
| 588 | Ochotona | MALPSPETLDSLDRVPASHPDEQHWTVCDNSDPILEVEAEGS |
| | princeps | MDVILVDDSPAPSGRDRIELEVKVNQRSIEDLCLCCGSSQVHR |
| | DNMT3L | QHPLFQGGLCAPCKDKFLEALFLYDEDGYQSYCSICGLGDTLL |
| | | VCESPDCTRGYCFACVDGLVGAGSSGHMHTVSPWVCFLCVP |
| | | GSRHGLLQRRRRWRTQLKVFHEQEAAQPLEIYETVPACRRKP |
| | | LRVLSLFEHIEKELASLGFLETGSSPGRIRHLDDVTDVVRRDVE |
| | | QWGPFDLVYGSTPPLGHASPRSPGWYLFQFHRMLQYTQPTAS |
| | | TQRPFFWMFVDNLLLTRDDLVTATRFLEVEPATLQDVRGRVL |
| | | QGAMRVWSNIPAVNSRHTELAPEAETALLAQSCRRAKASGEG |
| | | LARLLKSCFLPLREYFKYFPQSPLPLRK |
| 589 | Ochotona | QPLEIYETVPACRRKPLRVLSLFEHIEKELASLGFLETGSSPGRI |
| | princeps | RHLDDVTDVVRRDVEQWGPFDLVYGSTPPLGHASPRSPGWY |
| | DNMT3L catalytic | LFQFHRMLQYTQPTASTQRPFFWMFVDNLLLTRDDLVTATRF |
| | domain | LEVEPATLQDVRGRVLQGAMRVWSNIPAVNSRHTELAPEAET |
| | | ALLAQSCRRAKASGEGLARLLKSCFLPLREYFKYFPQSPLPLR |
| | | K |
| 590 | Neosciurus | MGGPRPAAVEESPHEIYKTVPAWKREPMRVLSLFGDIGKELTS |
| | carolinensis | LGFLETGSEAGRLKHLEDVTDTVRRDVEEWGPFDLVYGSTPA |
| | DNMT3L | LGHSCDRSPGWYLFQFHRLLQYARPRLGSPKPFFWMFVDNLL |
| | | LTKDDQAIASRFLEMEPVTLQDVHGRVLQNAVRVWTNVPAV |
| | | KSRHSALASEEELLLVQDGQRGRLPAQGPAALVKHCFLPLRE |
| | | YFKYFSQNTLPLYK |
| 591 | Neosciurus | SPHEIYKTVPAWKREPMRVLSLFGDIGKELTSLGFLETGSEAG |
| | carolinensis | RLKHLEDVTDTVRRDVEEWGPFDLVYGSTPALGHSCDRSPG |
| | DNMT3L catalytic | WYLFQFHRLLQYARPRLGSPKPFFWMFVDNLLLTKDDQAIAS |
| | domain | RFLEMEPVTLQDVHGRVLQNAVRVWTNVPAVKSRHSALASE |
| | | EELLLVQDGQRGRLPAQGPAALVKHCFLPLREYFKYFSQNTL |
| | | PLYK |
| 592 | Bison bison | MARSSPGTLNLEIMDGSDPDPALPPDREQWPPPCEILLDPEPEH |
| | DNMT3L | SLDIILVGSSELSSPPSPGPRRDFIAYEVKVNQRDIEDVCICCGS |
| | | LQLHTQHPLFEGGMCAPCKDKFLECLFLYDDDGYQSYCSICC |
| | | AGETLLICENPDCTRCYCFECVDTLVGPGTSGKVHAMSNWVC |
| | | FLCLPFPRSGLLQRRRKWRTWLKAFYDREAESPLVMYKTVPV |
| | | WKREPIRVLSLFGDIKKELTSLGFLEDGSKPGRLKHLDDVTNI |
| | | VRRDIDEWGPFDLTYGSTPTLGHTCDHPPGWYVYQFHRILQY |
| | | ARPLPGSPQPFFWMFVDNLVLTEEDLDVATRFLETDPVTIQDV |
| | | RGRTVQNAVHVWSNIPAVKSRHSALVSQEELSLLAQDRQRV |
| | | KSPVQGPATLVKNCFLPLREYFKYFSTELTSSL |
| 593 | Bison bison | SPLVMYKTVPVWKREPIRVLSLFGDIKKELTSLGFLEDGSKPG |
| | DNMT3L catalytic | RLKHLDDVTNIVRRDIDEWGPFDLTYGSTPTLGHTCDHPPGW |
| | domain | YVYQFHRILQYARPLPGSPQPFFWMFVDNLVLTEEDLDVATR |
| | | FLETDPVTIQDVRGRTVQNAVHVWSNIPAVKSRHSALVSQEE |
| | | LSLLAQDRQRVKSPVQGPATLVKNCFLPLREYFKYFSTELTSS |
| | | L |
| 594 | Equus przewalskii | MALSSPGTLSLETLDSWDPDVAGQLDEERWQPSSEIVGRPMA |
| | DNMT3L | AAPVLDLEEEPSMDIILVDSSELSSPPSPGPSRDMCICCGSFQVH |
| | | TQHPLFEGGMCAACKDKFLSCLFLYDDDGNQSYCSICCSGET |
| | | LLICENPDCTRCYCFECVDTLVSPRTSEKVQAMSNWVCFLCLP |
| | | FPRSGLLQRRRKWRGWLKAFYDQEAVRSRSAWGRRMRSGP |
| | | HLVGFLWLLVAKCPSALESPLEMYKTVPVWKREPVRVLSLFG |
| | | DIKKELTTLGFLENGSDPGRLKHLDDVTNTVRRDVEEWGPFD |
| | | LVYGSTPPLGHACDHPPGWYLFQFHRVLQYARPRPGSPQAFF |
| | | WMFVDNLVLTEDDRAVATRFLETDPVTIQDVCGRAVRNAVH |
| | | VWSNIPAVKSRHSALFSQEESFLRAQDRQRAKPPARGPAKLV |
| | | KNCFLPLREYFKYFSTEFTSSL |
| 595 | Equus przewalskii | SPLEMYKTVPVWKREPVRVLSLFGDIKKELTTLGFLENGSDPG |
| | DNMT3L catalytic | RLKHLDDVTNTVRRDVEEWGPFDLVYGSTPPLGHACDHPPG |
| | domain | WYLFQFHRVLQYARPRPGSPQAFFWMFVDNLVLTEDDRAVA |
| | | TRFLETDPVTIQDVCGRAVRNAVHVWSNIPAVKSRHSALFSQ |
| | | EESFLRAQDRQRAKPPARGPAKLVKNCFLPLREYFKYFSTEFT |
| | | SSL |
| 596 | Mus caroli | MGSRETPSSFSKTLETLDLETSDSSSPDADSPLEEQWLKSSPAL |
| | DNMT3L | KEDNVDMVLEDCKEPLSPSSPPTGREMIRYEVKVNRRSIEDIC |
| | | LCCGTLQVYTQHPLFEGGICAPCKDKFLESLFLYDDDGHQSY |
| | | CTICCSGGTLFICESPDCTRCYCFECVDILVGPGTSERINAMAC |
| | | WVCFLCLPFSRSGLLQRRKRWRHQLKAFHDQEGAGPMEIYK |
| | | TVSTWKRQPVRVLSLFGNIDKVLKSLGFLESGSGSGGGTLKY |
| | | VEDVTNVVRRDVEKWGPFDLVYGSTQPLGSSCDRCPGWYMF |
| | | QFHRILQYALPRQESQRPFFWIFMDNLLMTEDDQETTARFLQT |
| | | EAVTLQDVRGRDYQNVMRVWSNIPGLKSKHVPLTPKEEEYL |
| | | QAQVRTRSKLDAQKVDLLVKNCLLPLREYFKYFS |
| 597 | Mus caroli | GPMEIYKTVSTWKRQPVRVLSLFGNIDKVLKSLGFLESGSGSG |
| | DNMT3L catalytic | GGTLKYVEDVTNVVRRDVEKWGPFDLVYGSTQPLGSSCDRC |
| | domain | PGWYMFQFHRILQYALPRQESQRPFFWIFMDNLLMTEDDQET |
| | | TARFLQTEAVTLQDVRGRDYQNVMRVWSNIPGLKSKHVPLT |
| | | PKEEEYLQAQVRTRSKLDAQKVDLLVKNCLLPLREYFKYFS |
| 598 | Pan troglodytes | MAAIPALDPEAEPSMDVILVGSSELSSSISPRTGRDLIAYEVKA |
| | DNMT3L | NQRNIEDICICCGSLQVHTQHPLFEGGICAPCKDKSLDALFLYD |
| | | DDGYQSYCSICCSGETLLICGNPDCTRCYCFECVDSLVGPGTS |
| | | GKVHAMSNWVCYLCLPSSRSGLLQRRRKWRSQLKAFYDRES |
| | | ENPLEMFETVPVWRRQPVRVLSLFEDIKKELTSLGFLESGSDP |
| | | GQLKHVVDVTDTVRKDVEEWGPFDLVYGATPPLGHTCDRPP |
| | | SWYLFQFHRLLQYARPKPGSPRPFFWMFVDNLVLNKEDLDV |
| | | ASRFLEMEPVTIPDVHGGSLQNAVRVWSNIPAIRSSRHWALVS |
| | | EEELSLLAQNKQSSKLAAKWPTKLVKNCFLPLREYFKYFSTEL |
| | | TSSL |
| 599 | Pan troglodytes | NPLEMFETVPVWRRQPVRVLSLFEDIKKELTSLGFLESGSDPG |
| | DNMT3L catalytic | QLKHVVDVTDTVRKDVEEWGPFDLVYGATPPLGHTCDRPPS |
| | domain | WYLFQFHRLLQYARPKPGSPRPFFWMFVDNLVLNKEDLDVA |
| | | SRFLEMEPVTIPDVHGGSLQNAVRVWSNIPAIRSSRHWALVSE |
| | | EELSLLAQNKQSSKLAAKWPTKLVKNCFLPLREYFKYFSTELT |
| | | SSL |
| 600 | human TRDMT1 | MEPLRVLELYSGVGGMHHALRESCIPAQVVAAIDVNTVANEV |
| | (DNMT2) | YKYNFPHTQLLAKTIEGITLEEFDRLSFDMILMSPPCQPFTRIGR |
| | | QGDMTDSRTNSFLHILDILPRLQKLPKYILLENVKGFEVSSTRD |
| | | LLIQTIENCGFQYQEFLLSPTSLGIPNSRLRYFLIAKLQSEPLPFQ |
| | | APGQVLMEFPKIESVHPQKYAMDVENKIQEKNVEPNISFDGSI |
| | | QCSGKDAILFKLETAEEIHRKNQQDSDLSVKMLKDFLEDDTD |
| | | VNQYLLPPKSLLRYALLLDIVQPTCRRSVCFTKGYGSYIEGTG |
| | | SVLQTAEDVQVENIYKSLTNLSQEEQITKLLILKLRYFTPKEIA |
| | | NLLGFPPEFGFPEKITVKQRYRLLGNSLNVHVVAKLIKILYE |
| 601 | M. bacterium | MAEWYIPAIVSYQAIHNGFTLNKINHKIELQTMIDYLESKTLS |
| | methyltransferase | MNSKEPVKRGFWYKKHLDEIRIVYTAVKMSEQEGNIFDVRTL |
| | | FERGLSDIDLLTYSFPCQDLSQQGKQKGMGRDSQTRSGLLWEI |
| | | EKALDTSKKEDLPKYLLMENVVALTHKVNAEELDEWMMKL |
| | | ESLGYKNDLRILNAGDFGSSQARRRTFMISTLNEKVELPVGNK |
| | | KPKSMNKILNDEPTRKDFLPALDKFDLTEYKWTKSNINKAKLI |
| | | NYSTFNSEAYVYDSNFTGPTLTASGANSRIKFEYNGKIRKIGA |
| | | EEAYAYMGFKKSDYIKVNKLNYLNETKMIYTCGNSISVEVLR |
| | | SIMTNINNNFKENK |
| 602 | M. marinum | MLFLIGTFKYVLIYITKVIRIFEAFAGIGAQRKALRNIKSNYEVS |
| | methyltransferase | GMAEWYIPAIVSYQAIHNGFTLSRVDKKTKLTEMIKYLESKTL |
| | | SMDSKEPVRTGYWFKKHKDMVRIVYSAVKLSEAEGNIFDVR |
| | | TLHERKLEDIDLLTYSFPCQDLSQQGKQRGMKKDSGTRSGLL |
| | | WEIEKALEATPKDKLPKYLLMENVVALTHKTNKKDLDNWKR |
| | | KLRSLGYYNDINVLNAGDFGSSQARRRAFMISTLDSKVTLPLG |
| | | DKKPQAISKILNKETRSQDFMPALDEYEKTDFKRTLSNIKKCK |
| | | LIDYTSFNSEAYVYDPKYTGPTLTASGANSRIKFTHQGKMRKI |
| | | NAEEAYRYMGFSTNDYKKVNNLNFLSETKMIYTCGNSISVEV |
| | | LEEIMLKIIREDNNG |
| 603 | S. chinense | MKKIRLFEAFAGIGSQRRALKSVVGNNFEIAGLAEWYVPAIV |
| | methyltransferase | MYQIINNDFSKKNVLDNVPRDEVIDYLNSKCLSWDSKKPVSK |
| | | NFWNRKSQDILNVIYSAVKKSEEEGNIFDVRTLHERTLESIDIL |
| | | TYSFPCQDLSQQGIQKGMKKNSGTRSGLLWEIEKAIDNTPKN |
| | | NLPKILLMENVPALLNKTNELELKEWLIKLENMGYKNSIGILN |
| | | AADFGSPQARRRVFMISSRNKKIELPVGKSKPGKLNDILEKNV |
| | | EDKFIMTNLEKYDFSEFSLTKSNIKKCSLINYTKFNSEAYVYDP |
| | | DFTGPTLTASGANSRIKIYDKGFIRRMSPLESFRYMGFDDEDY |
| | | KKIDEFEFLTDTQKIFVCGNSISIEVLKAIFERIDSNE |
| 604 | M. penetrans M | MNSNKDKIKVIKVFEAFAGIGSQFKALKNIARSKNWEIQHSG |
| | MpeI | MVEWFVDAIVSYVAIHSKNFNPKIEQLDKDILSISNDSKMPISE |
| | | YGIKKINNTIKASYLNYAKKHENNLFDIKKVNKDNFPKNIDIFT |
| | | YSFPCQDLSVQGLQKGIDKELNTRSGLLWEIERILEEIKNSFSK |
| | | EEMPKYLLMENVKNLLSHKNKKNYNTWLKQLEKFGYKSKT |
| | | YLLNSKNFDNCQNRERVFCLSIRDDYLEKTGFKFKELEKVKN |
| | | PPKKIKDILVDSSNYKYLNLNKYETTTFRETKSNIISRSLKNYT |
| | | TFNSENYVYNINGIGPTLTASGANSRIKIETQQGVRYLTPLECF |
| | | KYMQFDVNDFKKVQSTNLISENKMIYIAGNSIPVKILEAIFNTL |
| | | EFVNNEE |
| 605 | S. monobiae | MSKVENKTKKLRVFEAFAGIGAQRKALEKVRKDEYEIVGLAE |
| | M SssI | WYVPAIVMYQAIHNNFHTKLEYKSVSREEMIDYLENKTLSWN |
| | | SKNPVSNGYWKRKKDDELKIIYNAIKLSEKEGNIFDIRDLYKR |
| | | TLKNIDLLTYSFPCQDLSQQGIQKGMKRGSGTRSGLLWEIERA |
| | | LDSTEKNDLPKYLLMENVGALLHKKNEEELNQWKQKLESLG |
| | | YQNSIEVLNAADFGSSQARRRVFMISTLNEFVELPKGDKKPKS |
| | | IKKVLNKIVSEKDILNNLLKYNLTEFKKTKSNINKASLIGYSKF |
| | | NSEGYVYDPEFTGPTLTASGANSRIKIKDGSNIRKMNSDETFL |
| | | YIGFDSQDGKRVNEIEFLTENQKIFVCGNSISVEVLEAIIDKIGG |
| 606 | H. parainfluenzae | MKDVLDDNLLEEPAAQYSLFEPESNPNLREKFTFIDLFAGIGG |
| | M HpaII | FRIAMQNLGGKCIFSSEWDEQAQKTYEANFGDLPYGDITLEET |
| | | KAFIPEKFDILCAGFPCQAFSIAGKRGGFEDTRGTLFFDVAEIIR |
| | | RHQPKAFFLENVKGLKNHDKGRTLKTILNVLREDLGYFVPEP |
| | | AIVNAKNFGVPQNRERIYIVGFHKSTGVNSFSYPEPLDKIVTFA |
| | | DIREEKTVPTKYYLSTQYIDTLRKHKERHESKGNGFGYEIIPDD |
| | | GIANAIVVGGMGRERNLVIDHRITDFTPTTNIKGEVNREGIRK |
| | | MTPREWARLQGFPDSYVIPVSDASAYKQFGNSVAVPAIQATG |
| | | KKILEKLGNLYD |
| 607 | A. luteus | MSKANAKYSFVDLFAGIGGFHAALAATGGVCEYAVEIDREA |
| | M AluI | AAVYERNWNKPALGDITDDANDEGVTLRGYDGPIDVLTGGF |
| | | PCQPFSKSGAQHGMAETRGTLFWNIARIIEEREPTVLILENVRN |
| | | LVGPRHRHEWLTIIETLRFFGYEVSGAPAIFSPHLLPAWMGGT |
| | | PQVRERVFITATLVPERMRDERIPRTETGEIDAEAIGPKPVATM |
| | | NDRFPIKKGGTELFHPGDRKSGWNLLTSGIIREGDPEPSNVDL |
| | | RLTETETLWIDAWDDLESTIRRATGRPLEGFPYWADSWTDFR |
| | | ELSRLVVIRGFQAPEREVVGDRKRYVARTDMPEGFVPASVTR |
| | | PAIDETLPAWKQSHLRRNYDFFERHFAEVVAWAYRWGVYTD |
| | | LFPASRRKLEWQAQDAPRLWDTVMHFRPSGIRAKRPTYLPAL |
| | | VAITQTSIVGPLERRLSPRETARLQGLPEWFDFGEQRAAATYK |
| | | QMGNGVNVGVVRHILREHVRRDRALLKLTPAGQRIINAVLAD |
| | | EPDATVGALGAAE |
| 608 | H. aegyptius M | MNLISLFSGAGGLDLGFQKAGFRIICANEYDKSIWKTYESNHS |
| | HaeIII | AKLIKGDISKISSDEFPKCDGIIGGPPCQSWSEGGSLRGIDDPRG |
| | | KLFYEYIRILKQKKPIFFLAENVKGMMAQRHNKAVQEFIQEFD |
| | | NAGYDVHIILLNANDYGVAQDRKRVFYIGFRKELNINYLPPIP |
| | | HLIKPTFKDVIWDLKDNPIPALDKNKTNGNKCIYPNHEYFIGS |
| | | YSTIFMSRNRVRQWNEPAFTVQASGRQCQLHPQAPVMLKVS |
| | | KNLNKFVEGKEHLYRRLTVRECARVQGFPDDFIFHYESLNDG |
| | | YKMIGNAVPVNLAYEIAKTIKSALEICKGN |
| 609 | H. haemolyticus M | MIEIKDKQLTGLRFIDLFAGLGGFRLALESCGAECVYSNEWDK |
| | HhaI | YAQEVYEMNFGEKPEGDITQVNEKTIPDHDILCAGFPCQAFSIS |
| | | GKQKGFEDSRGTLFFDIARIVREKKPKVVFMENVKNFASHDN |
| | | GNTLEVVKNTMNELDYSFHAKVLNALDYGIPQKRERIYMICF |
| | | RNDLNIQNFQFPKPFELNTFVKDLLLPDSEVEHLVIDRKDLVM |
| | | TNQEIEQTTPKTVRLGIVGKGGQGERIYSTRGIAITLSAYGGGI |
| | | FAKTGGYLVNGKTRKLHPRECARVMGYPDSYKVHPSTSQAY |
| | | KQFGNSVVINVLQYIAYNIGSSLNFKPY |
| 610 | Moraxella M MspI | MKPEILKLIRSKLDLTQKQASEIIEVSDKTWQQWESGKTEMHP |
| | | AYYSFLQEKLKDKINFEELSAQKTLQKKIFDKYNQNQITKNAE |
| | | ELAEITHIEERKDAYSSDFKFIDLFSGIGGIRQSFEVNGGKCVFS |
| | | SEIDPFAKFTYYTNFGVVPFGDITKVEATTIPQHDILCAGFPCQ |
| | | PFSHIGKREGFEHPTQGTMFHEIVRIIETKKTPVLFLENVPGLIN |
| | | HDDGNTLKVIIETLEDMGYKVHHTVLDASHFGIPQKRKRFYL |
| | | VAFLNQNIHFEFPKPPMISKDIGEVLESDVTGYSISEHLQKSYL |
| | | FKKDDGKPSLIDKNTTGAVKTLVSTYHKIQRLTGTFVKDGET |
| | | GIRLLTTNECKAIMGFPKDFVIPVSRTQMYRQMGNSVVVPVV |
| | | TKIAEQISLALKTVNQQSPQENFELELV |
| 611 | Ascobolus Masc1 | MSERRYEAGMTVALHEGSFLKIQRVYIRQYHADNRREHMLV |
| | | GPLFRRTKYLKALSKKVNEVAIVHESIHVPVQDVIGVRELIITN |
| | | RPFPECRKGDEHTGRLVCRWVYNLDERAKGREYKKQRYIRRI |
| | | TEAEADPEYRVEDRVLRRRWFQEGYIGDEISYKEHGNGDIVDI |
| | | RSESPLQVLDGWGGDLVDLENGEETSIPGPCRSASSYGRLMKP |
| | | PLAQAADSNTSRKYTFGDTFCGGGGVSLGARQAGLEVKWAF |
| | | DMNPNAGANYRRNFPNTDFFLAEAEQFIQLSVGISQHVDILHL |
| | | SPPCQTFSRAHTIAGKNDENNEASFFAVVNLIKAVRPRLFTVE |
| | | ETDGIMDRQSRQFIDTALMGITELGYSFRICVLNAIEYGVCQN |
| | | RKRLIIIGAAPGEELPPFPLPTHQDFFSKDPRRDLLPAVTLDDAL |
| | | STITPESTDHHLNHVWQPAEWKTPYDAHRPFKNAIRAGGGEY |
| | | DIYPDGRRKFTVRELACIQGFPDEYEFVGTLTDKRRIIGNAVPP |
| | | PLSAAIMSTLRQWMTEKDFERME |
| 612 | Arabidopsis MET1 | MVENGAKAAKRKKRPLPEIQEVEDVPRTRRPRRAAACTSFKE |
| | | KSIRVCEKSATIEVKKQQIVEEEFLALRLTALETDVEDRPTRRL |
| | | NDFVLFDSDGVPQPLEMLEIHDIFVSGAILPSDVCTDKEKEKG |
| | | VRCTSFGRVEHWSISGYEDGSPVIWISTELADYDCRKPAASYR |
| | | KVYDYFYEKARASVAVYKKLSKSSGGDPDIGLEELLAAVVRS |
| | | MSSGSKYFSSGAAIIDFVISQGDFIYNQLAGLDETAKKHESSYV |
| | | EIPVLVALREKSSKIDKPLQRERNPSNGVRIKEVSQVAESEALT |
| | | SDQLVDGTDDDRRYAILLQDEENRKSMQQPRKNSSSGSASNM |
| | | FYIKINEDEIANDYPLPSYYKTSEEETDELILYDASYEVQSEHLP |
| | | HRMLHNWALYNSDLRFISLELLPMKQCDDIDVNIFGSGVVTD |
| | | DNGSWISLNDPDSGSQSHDPDGMCIFLSQIKEWMIEFGSDDIISI |
| | | SIRTDVAWYRLGKPSKLYAPWWKPVLKTARVGISILTFLRVES |
| | | RVARLSFADVTKRLSGLQANDKAYISSDPLAVERYLVVHGQII |
| | | LQLFAVYPDDNVKRCPFVVGLASKLEDRHHTKWIIKKKKISL |
| | | KELNLNPRAGMAPVASKRKAMQATTTRLVNRIWGEFYSNYS |
| | | PEDPLQATAAENGEDEVEEEGGNGEEEVEEEGENGLTEDTVP |
| | | EPVEVQKPHTPKKIRGSSGKREIKWDGESLGKTSAGEPLYQQA |
| | | LVGGEMVAVGGAVTLEVDDPDEMPAIYFVEYMFESTDHCKM |
| | | LHGRFLQRGSMTVLGNAANERELFLTNECMTTQLKDIKGVAS |
| | | FEIRSRPWGHQYRKKNITADKLDWARALERKVKDLPTEYYC |
| | | KSLYSPERGGFFSLPLSDIGRSSGFCTSCKIREDEEKRSTIKLNV |
| | | SKTGFFINGIEYSVEDFVYVNPDSIGGLKEGSKTSFKSGRNIGL |
| | | RAYVVCQLLEIVPKESRKADLGSFDVKVRRFYRPEDVSAEKA |
| | | YASDIQELYFSQDTVVLPPGALEGKCEVRKKSDMPLSREYPIS |
| | | DHIFFCDLFFDTSKGSLKQLPANMKPKFSTIKDDTLLRKKKGK |
| | | GVESEIESEIVKPVEPPKEIRLATLDIFAGCGGLSHGLKKAGVS |
| | | DAKWAIEYEEPAGQAFKQNHPESTVFVDNCNVILRAIMEKGG |
| | | DQDDCVSTTEANELAAKLTEEQKSTLPLPGQVDFINGGPPCQG |
| | | FSGMNRFNQSSWSKVQCEMILAFLSFADYFRPRYFLLENVRTF |
| | | VSFNKGQTFQLTLASLLEMGYQVRFGILEAGAYGVSQSRKRA |
| | | FIWAAAPEEVLPEWPEPMHVFGVPKLKISLSQGLHYAAVRST |
| | | ALGAPFRPITVRDTIGDLPSVENGDSRTNKEYKEVAVSWFQKE |
| | | IRGNTIALTDHICKAMNELNLIRCKLIPTRPGADWHDLPKRKV |
| | | TLSDGRVEEMIPFCLPNTAERHNGWKGLYGRLDWQGNFPTSV |
| | | TDPQPMGKVGMCFHPEQHRILTVRECARSQGFPDSYEFAGNI |
| | | NHKHRQIGNAVPPPLAFALGRKLKEALHLKKSPQHQP |
| 613 | Ascobolus Masc2 | MELTPELSGVSTDLGGGGSIFAHWRMKEESPAPTEILDDLNVL |
| | | EWEKTTRDYSKEDLRIADQLFSIEDEHQSLPFETADAEDGTPT |
| | | EEEEEKELPMRTLDNFVLYDASDLELAALDLIGTELNIHAVGT |
| | | VGPIYTEGEEDEQEDEDEDVSPPVRTGTQATSASVTQMTVEL |
| | | YIRNIVQYEFCFNDDGTVETWIQTTNAHYKLLQPAKCYTSLY |
| | | RPVNDCLNVITAIITLAPESTTMSLKDLLKVMDDKAQAVSYEE |
| | | VERMSEFIVQHLDQWMETAPKKKSKLIEKSKVYIDLNNLAGI |
| | | DMVSGVRPPPVRRVTGRSSAPKKRIVRNMNDAVLLHQNETT |
| | | VTNWIHQLSAGMFGRALNVLGAETADVENLTCDPASAKFVV |
| | | PQRRLHKRLKWETRGHIPVSEEEYKHIYQGKKYAKFFEAVRA |
| | | VDESKLTIKLGDLVYVLDQDPKVTQTQFATAGREGRKKGAE |
| | | KEKIQVRFGRVLSIRQPDSNSKDAQNVFIHVQWLVLGCDTILQ |
| | | EMASRRELFLTDSCDTVFADVIYGVAKLTPLGAKDIPTVEFHE |
| | | SMATMMGENEFFVRFKYNYQDGSFTDLKDVDAEQIGTLQPR |
| | | VNTHRNPGYCSNCRIKYDNERTGDKWIYENDTEGEPRLFRSS |
| | | KGWCIYAQEFVYLQPVEKQPGTTFRVGYISEINKSSVIVELLA |
| | | RVDDDDKSGHISYSDPRHLYFTGTDIKVTFDKIIRKCFVFHDSG |
| | | DQKAKAPLMYGTLQRDLYYYRYEKRKGKAELVPVREIRSIHE |
| | | QTLNDWESRTQIERHGAVSGKKLKGLDIFAGCGGLTLGLDLS |
| | | GAVDTKWDIEFAPSAANTLALNFPDAQVFNQCANVLLSRAIQ |
| | | SEDEGSLDIEYDLQGRVLPDLPKKGEVDFIYGGPPCQGFSGVN |
| | | RYKKGNDIKNSLVATFLSYVDHYKPRFVLLENVKGLITTKLG |
| | | NSKNAEGKWEGGISNGVVKFIYRTLISMNYQCRIGLVQSGEY |
| | | GVPQSRPRVIFLAARMGERLPDLPEPMHAFEVLDSQYALPHIK |
| | | RYHTTQNGVAPLPRITIGEAVSDLPKFQYANPGVWPRHDPYSS |
| | | AKAQPSDKTIEKFSVSKATSFVGYLLQPYHSRPQSEFQRRLRT |
| | | KLVPSDEPAEKTSLLTTKLVTAHVTRLFNKETTQRIVCVPMWP |
| | | GADHRSLPKEMRPWCLVDPNSQAEKHRFWPGLFGRLGMEDF |
| | | FSTALTDVQPCGKQGKVLHPTQRRVYTVRELARAQGFPDWF |
| | | AFTDGDADSGLGGVKKWHRNIGNAVPVPLGEQIGRCIGYSV |
| | | WWKDDMIAQLREDGADEDEEMIDGNDQWVEELNTQMAAD |
| | | MPGLPLLVTHLLNLCVYRRLYGPNAKEFLPARVYDKKLEGGR |
| | | RRLVWAML |
| 614 | Neurospora Dim2 | MDSPDRSHGGMFIDVPAETMGFQEDYLDMFASVLSQGLAKE |
| | | GDYAHHQPLPAGKEECLEPIAVATTITPSPDDPQLQLQLELEQ |
| | | QFQTESGLNGVDPAPAPESEDEADLPDGFSDESPDDDFVVQRS |
| | | KHITVDLPVSTLINPRSTFQRIDENDNLVPPPQSTPERVAVEDL |
| | | LKAAKAAGKNKEDYIEFELHDFNFYVNYAYHPQEMRPIQLVA |
| | | TKVLHDKYYFDGVLKYGNTKHYVTGMQVLELPVGNYGASL |
| | | HSVKGQIWVRSKHNAKKEIYYLLKKPAFEYQRYYQPFLWIAD |
| | | LGKHVVDYCTRMVERKREVTLGCFKSDFIQWASKAHGKSKA |
| | | FQNWRAQHPSDDFRTSVAANIGYIWKEINGVAGAKRAAGDQ |
| | | LFRELMIVKPGQYFRQEVPPGPVVTEGDRTVAATIVTPYIKEC |
| | | FGHMILGKVLRLAGEDAEKEKEVKLAKRLKIENKNATKADT |
| | | KDDMKNDTATESLPTPLRSLPVQVLEATPIESDIVSIVSSDLPPS |
| | | ENNPPPLTNGSVKPKAKANPKPKPSTQPLHAAHVKYLSQELV |
| | | NKIKVGDVISTPRDDSSNTDTKWKPTDTDDHRWFGLVQRVHT |
| | | AKTKSSGRGLNSKSFDVIWFYRPEDTPCCAMKYKWRNELFLS |
| | | NHCTCQEGHHARVKGNEVLAVHPVDWFGTPESNKGEFFVRQ |
| | | LYESEQRRWITLQKDHLTCYHNQPPKPPTAPYKPGDTVLATLS |
| | | PSDKFSDPYEVVEYFTQGEKETAFVRLRKLLRRRKVDRQDAP |
| | | ANELVYTEDLVDVRAERIVGKCIMRCFRPDERVPSPYDRGGT |
| | | GNMFFITHRQDHGRCVPLDTLPPTLRQGFNPLGNLGKPKLRG |
| | | MDLYCGGGNFGRGLEEGGVVEMRWANDIWDKAIHTYMANT |
| | | PDPNKTNPFLGSVDDLLRLALEGKFSDNVPRPGEVDFIAAGSP |
| | | CPGFSLLTQDKKVLNQVKNQSLVASFASFVDFYRPKYGVLEN |
| | | VSGIVQTFVNRKQDVLSQLFCALVGMGYQAQLILGDAWAHG |
| | | APQSRERVFLYFAAPGLPLPDPPLPSHSHYRVKNRNIGFLCNG |
| | | ESYVQRSFIPTAFKFVSAGEGTADLPKIGDGKPDACVRFPDHR |
| | | LASGITPYIRAQYACIPTHPYGMNFIKAWNNGNGVMSKSDRD |
| | | LFPSEGKTRTSDASVGWKRLNPKTLFPTVTTTSNPSDARMGPG |
| | | LHWDEDRPYTVQEMRRAQGYLDEEVLVGRTTDQWKLVGNS |
| | | VSRHMALAIGLKFREAWLGTLYDESAVVATATATATTAAAV |
| | | GVTVPVMEEPGIGTTESSRPSRSPVHTAVDLDDSKSERSRSTTP |
| | | ATVLSTSSAAGDGSANAAGLEDDDNDDMEMMEVTRKRSSPA |
| | | VDEEGMRPSKVQKVEVTVASPASRRSSRQASRNPTASPSSKAS |
| | | KATTHEAPAPEELESDAESYSETYDKEGFDGDYHSGHEDQYS |
| | | EEDEEEEYAEPETMTVNGMTIVKL |
| 615 | Drosophila | MVFRVLELFSGIGGMHYAFNYAQLDGQIVAALDVNTVANAV |
| | dDnmt2 | YAHNYGSNLVKTRNIQSLSVKEVTKLQANMLLMSPPCQPHTR |
| | | QGLQRDTEDKRSDALTHLCGLIPECQELEYILMENVKGFESSQ |
| | | ARNQFIESLERSGFHWREFILTPTQFNVPNTRYRYYCIARKGA |
| | | DFPFAGGKIWEEMPGAIAQNQGLSQIAEIVEENVSPDFLVPDD |
| | | VLTKRVLVMDIIHPAQSRSMCFTKGYTHYTEGTGSAYTPLSED |
| | | ESHRIFELVKEIDTSNQDASKSEKILQQRLDLLHQVRLRYFTPR |
| | | EVARLMSFPENFEFPPETTNRQKYRLLGNSINVKVVGELIKLL |
| | | TIK |
| 616 | S. pombe Pmt1 | MLSTKRLRVLELYSGIGGMHYALNLANIPADIVCAIDINPQAN |
| | | EIYNLNHGKLAKHMDISTLTAKDFDAFDCKLWTMSPSCQPFT |
| | | RIGNRKDILDPRSQAFLNILNVLPHVNNLPEYILIENVQGFEES |
| | | KAAEECRKVLRNCGYNLIEGILSPNQFNIPNSRSRWYGLARLN |
| | | FKGEWSIDDVFQFSEVAQKEGEVKRIRDYLEIERDWSSYMVL |
| | | ESVLNKWGHQFDIVKPDSSSCCCFTRGYTHLVQGAGSILQMS |
| | | DHENTHEQFERNRMALQLRYFTAREVARLMGFPESLEWSKS |
| | | NVTEKCMYRLLGNSINVKVVSYLISLLLEPLNF |
| 617 | Arabidopsis DRM1 | MVMSHIFLISQIQEVEHGDSDDVNWNTDDDELAIDNFQFSPSP |
| | | VHISATSPNSIQNRISDETVASFVEMGFSTQMIARAIEETAGAN |
| | | MEPMMILETLFNYSASTEASSSKSKVINHFIAMGFPEEHVIKA |
| | | MQEHGDEDVGEITNALLTYAEVDKLRESEDMNININDDDDDN |
| | | LYSLSSDDEEDELNNSSNEDRILQALIKMGYLREDAAIAIERCG |
| | | EDASMEEVVDFICAAQMARQFDEIYAEPDKKELMNNNKKRR |
| | | TYTETPRKPNTDQLISLPKEMIGFGVPNHPGLMMHRPVPIPDIA |
| | | RGPPFFYYENVAMTPKGVWAKISSHLYDIVPEFVDSKHFCAA |
| | | ARKRGYIHNLPIQNRFQIQPPQHNTIQEAFPLTKRWWPSWDGR |
| | | TKLNCLLTCIASSRLTEKIREALERYDGETPLDVQKWVMYEC |
| | | KKWNLVWVGKNKLAPLDADEMEKLLGFPRDHTRGGGISTTD |
| | | RYKSLGNSFQVDTVAYHLSVLKPLFPNGINVLSLFTGIGGGEV |
| | | ALHRLQIKMNVVVSVEISDANRNILRSFWEQTNQKGILREFKD |
| | | VQKLDDNTIERLMDEYGGFDLVIGGSPCNNLAGGNRHHRVG |
| | | LGGEHSSLFFDYCRILEAVRRKARHMRR |
| 618 | Arabidopsis DRM2 | MVIWNNDDDDFLEIDNFQSSPRSSPIHAMQCRVENLAGVAVT |
| | | TSSLSSPTETTDLVQMGFSDEVFATLFDMGFPVEMISRAIKETG |
| | | PNVETSVIIDTISKYSSDCEAGSSKSKAIDHFLAMGFDEEKVVK |
| | | AIQEHGEDNMEAIANALLSCPEAKKLPAAVEEEDGIDWSSSD |
| | | DDTNYTDMLNSDDEKDPNSNENGSKIRSLVKMGFSELEASLA |
| | | VERCGENVDIAELTDFLCAAQMAREFSEFYTEHEEQKPRHNIK |
| | | KRRFESKGEPRSSVDDEPIRLPNPMIGFGVPNEPGLITHRSLPEL |
| | | ARGPPFFYYENVALTPKGVWETISRHLFEIPPEFVDSKYFCVA |
| | | ARKRGYIHNLPINNRFQIQPPPKYTIHDAFPLSKRWWPEWDKR |
| | | TKLNCILTCTGSAQLTNRIRVALEPYNEEPEPPKHVQRYVIDQ |
| | | CKKWNLVWVGKNKAAPLEPDEMESILGFPKNHTRGGGMSRT |
| | | ERFKSLGNSFQVDTVAYHLSVLKPIFPHGINVLSLFTGIGGGEV |
| | | ALHRLQIKMKLVVSVEISKVNRNILKDFWEQTNQTGELIEFSDI |
| | | QHLTNDTIEGLMEKYGGFDLVIGGSPCNNLAGGNRVSRVGLE |
| | | GDQSSLFFEYCRILEVVRARMRGS |
| 619 | Arabidopsis CMT1 | MAARNKQKKRAEPESDLCFAGKPMSVVESTIRWPHRYQSKK |
| | | TKLQAPTKKPANKGGKKEDEEIIKQAKCHFDKALVDGVLINL |
| | | NDDVYVTGLPGKLKFIAKVIELFEADDGVPYCRFRWYYRPED |
| | | TLIERFSHLVQPKRVFLSNDENDNPLTCIWSKVNIAKVPLPKIT |
| | | SRIEQRVIPPCDYYYDMKYEVPYLNFTSADDGSDASSSLSSDS |
| | | ALNCFENLHKDEKFLLDLYSGCGAMSTGFCMGASISGVKLIT |
| | | KWSVDINKFACDSLKLNHPETEVRNEAAEDFLALLKEWKRLC |
| | | EKFSLVSSTEPVESISELEDEEVEENDDIDEASTGAELEPGEFEV |
| | | EKFLGIMFGDPQGTGEKTLQLMVRWKGYNSSYDTWEPYSGL |
| | | GNCKEKLKEYVIDGFKSHLLPLPGTVYTVCGGPPCQGISGYNR |
| | | YRNNEAPLEDQKNQQLLVFLDIIDFLKPNYVLMENVVDLLRF |
| | | SKGFLARHAVASFVAMNYQTRLGMMAAGSYGLPQLRNRVFL |
| | | WAAQPSEKLPPYPLPTHEVAKKENTPKEFKDLQVGRIQMEFL |
| | | KLDNALTLADAISDLPPVTNYVANDVMDYNDAAPKTEFENFI |
| | | SLKRSETLLPAFGGDPTRRLFDHQPLVLGDDDLERVSYIPKQK |
| | | GANYRDMPGVLVHNNKAEINPRFRAKLKSGKNVVPAYAISFI |
| | | KGKSKKPFGRLWGDEIVNTVVTRAEPHNQCVIHPMQNRVLSV |
| | | RENARLQGFPDCYKLCGTIKEKYIQVGNAVAVPVGVALGYAF |
| | | GMASQGLTDDEPVIKLPFKYPECMQAKDQI |
| 620 | Arabidopsis CMT2 | MLSPAKCESEEAQAPLDLHSSSRSEPECLSLVLWCPNPEEAAP |
| | | SSTRELIKLPDNGEMSLRRSTTLNCNSPEENGGEGRVSQRKSS |
| | | RGKSQPLLMLTNGCQLRRSPRFRALHANFDNVCSVPVTKGGV |
| | | SQRKFSRGKSQPLLTLTNGCQLRRSPRFRAVDGNFDSVCSVPV |
| | | TGKFGSRKRKSNSALDKKESSDSEGLTFKDIAVIAKSLEMEIIS |
| | | ECQYKNNVAEGRSRLQDPAKRKVDSDTLLYSSINSSKQSLGS |
| | | NKRMRRSQRFMKGTENEGEENLGKSKGKGMSLASCSFRRST |
| | | RLSGTVETGNTETLNRRKDCGPALCGAEQVRGTERLVQISKK |
| | | DHCCEAMKKCEGDGLVSSKQELLVFPSGCIKKTVNGCRDRTL |
| | | GKPRSSGLNTDDIHTSSLKISKNDTSNGLTMTTALVEQDAMES |
| | | LLQGKTSACGAADKGKTREMHVNSTVIYLSDSDEPSSIEYLNG |
| | | DNLTQVESGSALSSGGNEGIVSLDLNNPTKSTKRKGKRVTRT |
| | | AVQEQNKRSICFFIGEPLSCEEAQERWRWRYELKERKSKSRG |
| | | QQSEDDEDKIVANVECHYSQAKVDGHTFSLGDFAYIKGEEEE |
| | | THVGQIVEFFKTTDGESYFRVQWFYRATDTIMERQATNHDKR |
| | | RLFYSTVMNDNPVDCLISKVTVLQVSPRVGLKPNSIKSDYYFD |
| | | MEYCVEYSTFQTLRNPKTSENKLECCADVVPTESTESILKKKS |
| | | FSGELPVLDLYSGCGGMSTGLSLGAKISGVDVVTKWAVDQN |
| | | TAACKSLKLNHPNTQVRNDAAGDFLQLLKEWDKLCKRYVEN |
| | | NDQRTDTLRSVNSTKETSGSSSSSDDDSDSEEYEVEKLVDICF |
| | | GDHDKTGKNGLKFKVHWKGYRSDEDTWELAEELSNCQDAIR |
| | | EFVTSGFKSKILPLPGRVGVICGGPPCQGISGYNRHRNVDSPLN |
| | | DERNQQIIVFMDIVEYLKPSYVLMENVVDILRMDKGSLGRYA |
| | | LSRLVNMRYQARLGIMTAGCYGLSQFRSRVFMWGAVPNKNL |
| | | PPFPLPTHDVIVRYGLPLEFERNVVAYAEGQPRKLEKALVLKD |
| | | AISDLPHVSNDEDREKLPYESLPKTDFQRYIRSTKRDLTGSAID |
| | | NCNKRTMLLHDHRPFHINEDDYARVCQIPKRKGANFRDLPGL |
| | | IVRNNTVCRDPSMEPVILPSGKPLVPGYVFTFQQGKSKRPFAR |
| | | LWWDETVPTVLTVPTCHSQALLHPEQDRVLTIRESARLQGFP |
| | | DYFQFCGTIKERYCQIGNAVAVSVSRALGYSLGMAFRGLARD |
| | | EHLIKLPQNFSHSTYPQLQETIPH |
| 621 | Arabidopsis CMT3 | MAPKRKRPATKDDTTKSIPKPKKRAPKRAKTVKEEPVTVVEE |
| | | GEKHVARFLDEPIPESEAKSTWPDRYKPIEVQPPKASSRKKTK |
| | | DDEKVEIIRARCHYRRAIVDERQIYELNDDAYVQSGEGKDPFI |
| | | CKIIEMFEGANGKLYFTARWFYRPSDTVMKEFEILIKKKRVFF |
| | | SEIQDTNELGLLEKKLNILMIPLNENTKETIPATENCDFFCDMN |
| | | YFLPYDTFEAIQQETMMAISESSTISSDTDIREGAAAISEIGECS |
| | | QETEGHKKATLLDLYSGCGAMSTGLCMGAQLSGLNLVTKW |
| | | AVDMNAHACKSLQHNHPETNVRNMTAEDFLFLLKEWEKLCI |
| | | HFSLRNSPNSEEYANLHGLNNVEDNEDVSEESENEDDGEVFT |
| | | VDKIVGISFGVPKKLLKRGLYLKVRWLNYDDSHDTWEPIEGL |
| | | SNCRGKIEEFVKLGYKSGILPLPGGVDVVCGGPPCQGISGHNR |
| | | FRNLLDPLEDQKNKQLLVYMNIVEYLKPKFVLMENVVDMLK |
| | | MAKGYLARFAVGRLLQMNYQVRNGMMAAGAYGLAQFRLR |
| | | FFLWGALPSEIIPQFPLPTHDLVHRGNIVKEFQGNIVAYDEGHT |
| | | VKLADKLLLKDVISDLPAVANSEKRDEITYDKDPTTPFQKFIR |
| | | LRKDEASGSQSKSKSKKHVLYDHHPLNLNINDYERVCQVPKR |
| | | KGANFRDFPGVIVGPGNVVKLEEGKERVKLESGKTLVPDYAL |
| | | TYVDGKSCKPFGRLWWDEIVPTVVTRAEPHNQVIIHPEQNRV |
| | | LSIRENARLQGFPDDYKLFGPPKQKYIQVGNAVAVPVAKALG |
| | | YALGTAFQGLAVGKDPLLTLPEGFAFMKPTLPSELA |
| 622 | Neurospora Rid | MAEQNPFVIDDEDDVIQIHDEEEVEEEVAEVIDITEDDIEPSEL |
| | | DRAFGSRPKEETLPSLLLRDQGFIVRPGMTVELKAPIGRFAISF |
| | | VRVNSIVKVRQAHVNNVTIRGHGFTRAKEMNGMLPKQLNEC |
| | | CLVASIDTRDPRP |
| 623 | E. coli strain 12 | MNNNDLVAKLWKLCDNLRDGGVSYQNYVNELASLLFLKMC |
| | hsdM | KETGQEAEYLPEGYRWDDLKSRIGQEQLQFYRKMLVHLGED |
| | | DKKLVQAVFHNVSTTITEPKQITALVSNMDSLDWYNGAHGKS |
| | | RDDFGDMYEGLLQKNANETKSGAGQYFTPRPLIKTIIHLLKPQ |
| | | PREVVQDPAAGTAGFLIEADRYVKSQTNDLDDLDGDTQDFQI |
| | | HRAFIGLELVPGTRRLALMNCLLHDIEGNLDHGGAIRLGNTLG |
| | | SDGENLPKAHIVATNPPFGSAAGTNITRTFVHPTSNKQLCFMQ |
| | | HIIETLHPGGRAAVVVPDNVLFEGGKGTDIRRDLMDKCHLHTI |
| | | LRLPTGIFYAQGVKTNVLFFTKGTVANPNQDKNCTDDVWVY |
| | | DLRTNMPSFGKRTPFTDEHLQPFERVYGEDPHGLSPRTEGEWS |
| | | FNAEETEVADSEENKNTDQHLATSRWRKFSREWIRTAKSDSL |
| | | DISWLKDKDSIDADSLPEPDVLAAEAMGELVQALSELDALMR |
| | | ELGASDEADLQRQLLEEAFGGVKE |
| 624 | E. coli strain 12 | MSAGKLPEGWVIAPVSTVTTLIRGVTYKKEQAINYLKDDYLP |
| | hsdS | LIRANNIQNGKFDTTDLVFVPKNLVKESQKISPEDIVIAMSSGS |
| | | KSVVGKSAHQHLPFECSFGAFCGVLRPEKLIFSGFIAHFTKSSL |
| | | YRNKISSLSAGANINNIKPASFDLINIPIPPLAEQKIIAEKLDTLL |
| | | AQVDSTKARFEQIPQILKRFRQAVLGGAVNGKLTEKWRNFEP |
| | | QHSVFKKLNFESILTELRNGLSSKPNESGVGHPILRISSVRAGH |
| | | VDQNDIRFLECSESELNRHKLQDGDLLFTRYNGSLEFVGVCGL |
| | | LKKLQHQNLLYPDKLIRARLTKDALPEYIEIFFSSPSARNAMM |
| | | NCVKTTSGQKGISGKDIKSQVVLLPPVKEQAEIVRRVEQLFAY |
| | | ADTIEKQVNNALARVNNLTQSILAKAFRGELTAQWRAENPDL |
| | | ISGENSAAALLEKIKAERAASGGKKASRKKS |
| 625 | T. aquaticus M | MGLPPLLSLPSNSAPRSLGRVETPPEVVDFMVSLAEAPRGGRV |
| | TagI | LEPACAHGPFLRAFREAHGTAYRFVGVEIDPKALDLPPWAEGI |
| | | LADFLLWEPGEAFDLILGNPPYGIVGEASKYPIHVFKAVKDLY |
| | | KKAFSTWKGKYNLYGAFLEKAVRLLKPGGVLVFVVPATWLV |
| | | LEDFALLREFLAREGKTSVYYLGEVFPQKKVSAVVIRFQKSGK |
| | | GLSLWDTQESESGFTPILWAEYPHWEGEIIRFETEETRKLEISG |
| | | MPLGDLFHIRFAARSPEFKKHPAVRKEPGPGLVPVLTGRNLKP |
| | | GWVDYEKNHSGLWMPKERAKELRDFYATPHLVVAHTKGTR |
| | | VVAAWDERAYPWREEFHLLPKEGVRLDPSSLVQWLNSEAMQ |
| | | KHVRTLYRDFVPHLTLRMLERLPVRREYGFHTSPESARNF |
| 626 | E. coli M EcoDam | MKKNRAFLKWAGGKYPLLDDIKRHLPKGECLVEPFVGAGSV |
| | | FLNTDFSRYILADINSDLISLYNIVKMRTDEYVQAARELFVPET |
| | | NCAEVYYQFREEFNKSQDPFRRAVLFLYLNRYGYNGLCRYNL |
| | | RGEFNVPFGRYKKPYFPEAELYHFAEKAQNAFFYCESYADSM |
| | | ARADDASVVYCDPPYAPLSATANFTAYHTNSFTLEQQAHLAE |
| | | IAEGLVERHIPVLISNHDTMLTREWYQRAKLHVVKVRRSISSN |
| | | GGTRKKVDELLALYKPGVVSPAKK |
| 627 | C. crescentus M | MKFGPETIIHGDCIEQMNALPEKSVDLIFADPPYNLQLGGDLL |
| | CcrMI | RPDNSKVDAVDDHWDQFESFAAYDKFTREWLKAARRVLKD |
| | | DGAIWVIGSYHNIFRVGVAVQDLGFWILNDIVWRKSNPMPNF |
| | | KGTRFANAHETLIWASKSQNAKRYTFNYDALKMANDEVQM |
| | | RSDWTIPLCTGEERIKGADGQKAHPTQKPEALLYRVILSTTKP |
| | | GDVILDPFFGVGTTGAAAKRLGRKFIGIEREAEYLEHAKARIA |
| | | KVVPIAPEDLDVMGSKRAEPRVPFGTIVEAGLLSPGDTLYCSK |
| | | GTHVAKVRPDGSITVGDLSGSIHKIGALVQSAPACNGWTYWH |
| | | FKTDAGLAPIDVLRAQVRAGMN |
| 628 | C. difficile CamA | MDDISQDNFLLSKEYENSLDVDTKKASGIYYTPKIIVDYIVKK |
| | | TLKNHDIIKNPYPRILDISCGCGNFLLEVYDILYDLFEENIYELK |
| | | KKYDENYWTVDNIHRHILNYCIYGADIDEKAISILKDSLTNKK |
| | | VVNDLDESDIKINLFCCDSLKKKWRYKFDYIVGNPPYIGHKKL |
| | | EKKYKKFLLEKYSEVYKDKADLYFCFYKKIIDILKQGGIGSVIT |
| | | PRYFLESLSGKDLREYIKSNVNVQEIVDFLGANIFKNIGVSSCIL |
| | | TFDKKKTKETYIDVFKIKNEDICINKFETLEELLKSSKFEHFNIN |
| | | QRLLSDEWILVNKDDETFYNKIQEKCKYSLEDIAISFQGIITGC |
| | | DKAFILSKDDVKLNLVDDKFLKCWIKSKNINKYIVDKSEYRLI |
| | | YSNDIDNENTNKRILDEIIGLYKTKLENRRECKSGIRKWYELQ |
| | | WGREKLFFERKKIMYPYKSNENRFAIDYDNNFSSADVYSFFIK |
| | | EEYLDKFSYEYLVGILNSSVYDKYFKITAKKMSKNIYDYYPN |
| | | KVMKIRIFRDNNYEEIENLSKQIISILLNKSIDKGKVEKLQIKMD |
| | | NLIMDSLGI |
| 629 | KAP1 | MAASAAAASAAAASAASGSPGPGEGSAGGEKRSTAPSAAAS |
| | | ASASAAASSPAGGGAEALELLEHCGVCRERLRPEREPRLLPCL |
| | | HSACSACLGPAAPAAANSSGDGGAAGDGTVVDCPVCKQQCF |
| | | SKDIVENYFMRDSGSKAATDAQDANQCCTSCEDNAPATSYC |
| | | VECSEPLCETCVEAHQRVKYTKDHTVRSTGPAKSRDGERTVY |
| | | CNVHKHEPLVLFCESCDTLTCRDCQLNAHKDHQYQFLEDAV |
| | | RNQRKLLASLVKRLGDKHATLQKSTKEVRSSIRQVSDVQKRV |
| | | QVDVKMAILQIMKELNKRGRVLVNDAQKVTEGQQERLERQH |
| | | WTMTKIQKHQEHILRFASWALESDNNTALLLSKKLIYFQLHR |
| | | ALKMIVDPVEPHGEMKFQWDLNAWTKSAEAFGKIVAERPGT |
| | | NSTGPAPMAPPRAPGPLSKQGSGSSQPMEVQEGYGFGSGDDP |
| | | YSSAEPHVSGVKRSRSGEGEVSGLMRKVPRVSLERLDLDLTA |
| | | DSQPPVFKVFPGSTTEDYNLIVIERGAAAAATGQPGTAPAGTP |
| | | GAPPLAGMAIVKEEETEAAIGAPPTATEGPETKPVLMALAEGP |
| | | GAEGPRLASPSGSTSSGLEVVAPEGTSAPGGGPGTLDDSATIC |
| | | RVCQKPGDLVMCNQCEFCFHLDCHLPALQDVPGEEWSCSLC |
| | | HVLPDLKEEDGSLSLDGADSTGVVAKLSPANQRKCERVLLAL |
| | | FCHEPCRPLHQLATDSTFSLDQPGGTLDLTLIRARLQEKLSPPY |
| | | SSPQEFAQDVGRMFKQFNKLTEDKADVQSIIGLQRFFETRMNE |
| | | AFGDTKFSAVLVEPPPMSLPGAGLSSQELSGGPGDGP |
| 630 | MECP2 | MVAGMLGLREEKSEDQDLQGLKDKPLKFKKVKKDKKEEKE |
| | | GKHEPVQPSAHHSAEPAEAGKAETSEGSGSAPAVPEASASPK |
| | | QRRSIIRDRGPMYDDPTLPEGWTRKLKQRKSGRSAGKYDVYL |
| | | INPQGKAFRSKVELIAYFEKVGDTSLDPNDFDFTVTGRGSPSR |
| | | REQKPPKKPKSPKAPGTGRGRGRPKGSGTTRPKAATSEGVQV |
| | | KRVLEKSPGKLLVKMPFQTSPGGKAEGGGATTSTQVMVIKRP |
| | | GRKRKAEADPQAIPKKRGRKPGSVVAAAAAEAKKKAVKESSI |
| | | RSVQETVLPIKKRKTRETVSIEVKEVVKPLLVSTLGEKSGKGL |
| | | KTCKSPGRKSKESSPKGRSSSASSPPKKEHHHHHHHSESPKAP |
| | | VPLLPPLPPPPPEPESSEDPTSPPEPQDLSSSVCKEEKMPRGGSL |
| | | ESDGCPKEPAKTQPAVATAATAAEKYKHRGEGERKDIVSSSM |
| | | PRPNREEPVDSRTPVTERVS |
| 631 | linker | SGGS |
| 632 | linker | SGGSSGSETPGTSESATPESSGGS |
| 633 | linker | SGGSSGGSSGSETPGTSESATPESSGGSSGGS |
| 634 | linker | GGSGGSPGSPAGSPTSTEEGTSESATPESGPGTSTEPSEGSAPGS |
| | | PAGSPTSTEEGTSTEPSEGSAPGTSTEPSEGSAPGTSESATPESG |
| | | PGSEPATSGGSGGS |
| 635 | G linker | GSGGG |
| 636 | GX4 linker | GGGGSGGGGSGGGGSGGGGS |
| 637 | W linker | SSGNSNANSRGPSFSSGLVPLSLRGSH |
| 638 | XTEN linker | SGSETPGTSESATPES |
| | (XTEN16) | |
| 639 | XTEN linker | SGGSSGGSSGSETPGTSESATPES |
| 640 | XTEN linker | SGGSSGGSSGSETPGTSESATPESSGGSSGGSSGGSSGGS |
| 641 | XTEN linker | SGGSSGGSSGSETPGTSESATPESSGGSSGGSSGGSSGGSSGSE |
| | | TPGTSESATPESSGGSSGGS |
| 642 | XTEN linker | PGSPAGSPTSTEEGTSESATPESGPGTSTEPSEGSAPGSPAGSPT |
| | | STEEGTSTEPSEGSAPGTSTEPSEGSAPGTSESATPESGPGSEPA |
| | | TS |
| 643 | XTEN linker | GGPSSGAPPPSGGSPAGSPTSTEEGTSESATPESGPGTSTEPSEG |
| | (XTEN80) | SAPGSPAGSPTSTEEGTSTEPSEGSAPGTSTEPSE |
| 644 | NLS | PKKKRKV |
| 645 | NLS | AVKRPAATKKAGQAKKKKLD |
| 646 | NLS | MSRRRKANPTKLSENAKKLAKEVEN |
| 647 | NLS | PAAKRVKLD |
| 648 | NLS | KLKIKRPVK |
| 649 | NLS | MDSLLMNRRKFLYQFKNVRWAKGRRETYLC |
| 660 | fusion protein | MGTMPKKKRKVPKKKRKVYNHDQEFDPPKVYPPVPAEKRKP |
| | (Configuration 7) | IRVLSLFDGIATGLLVLKDLGIQVDRYIASEVCEDSITVGMVRH |
| | | QGKIMYVGDVRSVTQKHIQEWGPFDLVIGGSPCNDLSIVNPA |
| | | RKGLYEGTGRLFFEFYRLLHDARPKEGDDRPFFWLFENVVAM |
| | | GVSDKRDISRFLESNPVMIDAKEVSAAHRARYFWGNLPGMNR |
| | | PLASTVNDKLELQECLEHGRIAKFSKVRTITTRSNSIKQGKDQ |
| | | HFPVFMNEKEDILWCTEMERVFGFPVHYTDVSNMSRLARQRL |
| | | LGRSWSVPVIRHLFAPLKEYFACVSSGNSNANSRGPSFSSGLV |
| | | PLSLRGSHMAAIPALDPEAEPSMDVILVGSSELSSSVSPGTGRD |
| | | LIAYEVKANQRNIEDICICCGSLQVHTQHPLFEGGICAPCKDKF |
| | | LDALFLYDDDGYQSYCSICCSGETLLICGNPDCTRCYCFECVD |
| | | SLVGPGTSGKVHAMSNWVCYLCLPSSRSGLLQRRRKWRSQL |
| | | KAFYDRESENPLEMFETVPVWRRQPVRVLSLFEDIKKELTSLG |
| | | FLESGSDPGQLKHVVDVTDTVRKDVEEWGPFDLVYGATPPLG |
| | | HTCDRPPSWYLFQFHRLLQYARPKPGSPRPFFWMFVDNLVLN |
| | | KEDLDVASRFLEMEPVTIPDVHGGSLQNAVRVWSNIPAIRSRH |
| | | WALVSEEELSLLAQNKQSSKLAAKWPTKLVKNCFLPLREYFK |
| | | YFSTELTSSLGGPSSGAPPPSGGSPAGSPTSTEEGTSESATPESG |
| | | PGTSTEPSEGSAPGSPAGSPTSTEEGTSTEPSEGSAPGTSTEPSE |
| | | LEDKKYSIGLAIGTNSVGWAVITDEYKVPSKKFKVLGNTDRH |
| | | SIKKNLIGALLFDSGETAEATRLKRTARRRYTRRKNRICYLQEI |
| | | FSNEMAKVDDSFFHRLEESFLVEEDKKHERHPIFGNIVDEVAY |
| | | HEKYPTIYHLRKKLVDSTDKADLRLIYLALAHMIKFRGHFLIE |
| | | GDLNPDNSDVDKLFIQLVQTYNQLFEENPINASGVDAKAILSA |
| | | RLSKSRRLENLIAQLPGEKKNGLFGNLIALSLGLTPNFKSNFDL |
| | | AEDAKLQLSKDTYDDDLDNLLAQIGDQYADLFLAAKNLSDAI |
| | | LLSDILRVNTEITKAPLSASMIKRYDEHHQDLTLLKALVRQQL |
| | | PEKYKEIFFDQSKNGYAGYIDGGASQEEFYKFIKPILEKMDGT |
| | | EELLVKLNREDLLRKQRTFDNGSIPHQIHLGELHAILRRQEDF |
| | | YPFLKDNREKIEKILTFRIPYYVGPLARGNSRFAWMTRKSEETI |
| | | TPWNFEEVVDKGASAQSFIERMTNFDKNLPNEKVLPKHSLLY |
| | | EYFTVYNELTKVKYVTEGMRKPAFLSGEQKKAIVDLLFKTNR |
| | | KVTVKQLKEDYFKKIECFDSVEISGVEDRFNASLGTYHDLLKII |
| | | KDKDFLDNEENEDILEDIVLTLTLFEDREMIEERLKTYAHLFD |
| | | DKVMKQLKRRRYTGWGRLSRKLINGIRDKQSGKTILDFLKSD |
| | | GFANRNFMQLIHDDSLTFKEDIQKAQVSGQGDSLHEHIANLA |
| | | GSPAIKKGILQTVKVVDELVKVMGRHKPENIVIEMARENQTT |
| | | QKGQKNSRERMKRIEEGIKELGSQILKEHPVENTQLQNEKLYL |
| | | YYLQNGRDMYVDQELDINRLSDYDVDAIVPQSFLKDDSIDNK |
| | | VLTRSDKNRGKSDNVPSEEVVKKMKNYWRQLLNAKLITQRK |
| | | FDNLTKAERGGLSELDKAGFIKRQLVETRQITKHVAQILDSRM |
| | | NTKYDENDKLIREVKVITLKSKLVSDFRKDFQFYKVREINNYH |
| | | HAHDAYLNAVVGTALIKKYPKLESEFVYGDYKVYDVRKMIA |
| | | KSEQEIGKATAKYFFYSNIMNFFKTEITLANGEIRKRPLIETNG |
| | | ETGEIVWDKGRDFATVRKVLSMPQVNIVKKTEVQTGGFSKES |
| | | ILPKRNSDKLIARKKDWDPKKYGGFDSPTVAYSVLVVAKVEK |
| | | GKSKKLKSVKELLGITIMERSSFEKNPIDFLEAKGYKEVKKDLI |
| | | IKLPKYSLFELENGRKRMLASAGELQKGNELALPSKYVNFLY |
| | | LASHYEKLKGSPEDNEQKQLFVEQHKHYLDEIIEQISEFSKRVI |
| | | LADANLDKVLSAYNKHRDKPIREQAENIIHLFTLTNLGAPAAF |
| | | KYFDTTIDRKRYTSTKEVLDATLIHQSITGLYETRIDLSQLGGD |
| | | SPKKKRKVGVDGSSGSETPGTSESATPESRTLVTFKDVFVDFT |
| | | REEWKLLDTAQQIVYRNVMLENYKNLVSLGYQLTKPDVILRL |
| | | EKGEEPSADYKDDDDKAPKKKRKVPKKKRKV |
| 661 | fusion protein | MGTMPKKKRKVPKKKRKVYNHDQEFDPPKVYPPVPAEKRKP |
| | (Configuration 9) | IRVLSLFDGIATGLLVLKDLGIQVDRYIASEVCEDSITVGMVRH |
| | | QGKIMYVGDVRSVTQKHIQEWGPFDLVIGGSPCNDLSIVNPA |
| | | RKGLYEGTGRLFFEFYRLLHDARPKEGDDRPFFWLFENVVAM |
| | | GVSDKRDISRFLESNPVMIDAKEVSAAHRARYFWGNLPGMNR |
| | | PLASTVNDKLELQECLEHGRIAKFSKVRTITTRSNSIKQGKDQ |
| | | HFPVFMNEKEDILWCTEMERVFGFPVHYTDVSNMSRLARQRL |
| | | LGRSWSVPVIRHLFAPLKEYFACVSSGNSNANSRGPSFSSGLV |
| | | PLSLRGSHMAAIPALDPEAEPSMDVILVGSSELSSSVSPGTGRD |
| | | LIAYEVKANQRNIEDICICCGSLQVHTQHPLFEGGICAPCKDKF |
| | | LDALFLYDDDGYQSYCSICCSGETLLICGNPDCTRCYCFECVD |
| | | SLVGPGTSGKVHAMSNWVCYLCLPSSRSGLLQRRRKWRSQL |
| | | KAFYDRESENPLEMFETVPVWRRQPVRVLSLFEDIKKELTSLG |
| | | FLESGSDPGQLKHVVDVTDTVRKDVEEWGPFDLVYGATPPLG |
| | | HTCDRPPSWYLFQFHRLLQYARPKPGSPRPFFWMFVDNLVLN |
| | | KEDLDVASRFLEMEPVTIPDVHGGSLQNAVRVWSNIPAIRSRH |
| | | WALVSEEELSLLAQNKQSSKLAAKWPTKLVKNCFLPLREYFK |
| | | YFSTELTSSLGGPSSGAPPPSGGSPAGSPTSTEEGTSESATPESG |
| | | PGTSTEPSEGSAPGSPAGSPTSTEEGTSTEPSEGSAPGTSTEPSE |
| | | LEDKKYSIGLAIGTNSVGWAVITDEYKVPSKKFKVLGNTDRH |
| | | SIKKNLIGALLFDSGETAEATRLKRTARRRYTRRKNRICYLQEI |
| | | FSNEMAKVDDSFFHRLEESFLVEEDKKHERHPIFGNIVDEVAY |
| | | HEKYPTIYHLRKKLVDSTDKADLRLIYLALAHMIKFRGHFLIE |
| | | GDLNPDNSDVDKLFIQLVQTYNQLFEENPINASGVDAKAILSA |
| | | RLSKSRRLENLIAQLPGEKKNGLFGNLIALSLGLTPNFKSNFDL |
| | | AEDAKLQLSKDTYDDDLDNLLAQIGDQYADLFLAAKNLSDAI |
| | | LLSDILRVNTEITKAPLSASMIKRYDEHHQDLTLLKALVRQQL |
| | | PEKYKEIFFDQSKNGYAGYIDGGASQEEFYKFIKPILEKMDGT |
| | | EELLVKLNREDLLRKQRTFDNGSIPHQIHLGELHAILRRQEDF |
| | | YPFLKDNREKIEKILTFRIPYYVGPLARGNSRFAWMTRKSEETI |
| | | TPWNFEEVVDKGASAQSFIERMTNFDKNLPNEKVLPKHSLLY |
| | | EYFTVYNELTKVKYVTEGMRKPAFLSGEQKKAIVDLLFKTNR |
| | | KVTVKQLKEDYFKKIECFDSVEISGVEDRFNASLGTYHDLLKII |
| | | KDKDFLDNEENEDILEDIVLTLTLFEDREMIEERLKTYAHLFD |
| | | DKVMKQLKRRRYTGWGRLSRKLINGIRDKQSGKTILDFLKSD |
| | | GFANRNFMQLIHDDSLTFKEDIQKAQVSGQGDSLHEHIANLA |
| | | GSPAIKKGILQTVKVVDELVKVMGRHKPENIVIEMARENQTT |
| | | QKGQKNSRERMKRIEEGIKELGSQILKEHPVENTQLQNEKLYL |
| | | YYLQNGRDMYVDQELDINRLSDYDVDAIVPQSFLKDDSIDNK |
| | | VLTRSDKNRGKSDNVPSEEVVKKMKNYWRQLLNAKLITQRK |
| | | FDNLTKAERGGLSELDKAGFIKRQLVETRQITKHVAQILDSRM |
| | | NTKYDENDKLIREVKVITLKSKLVSDFRKDFQFYKVREINNYH |
| | | HAHDAYLNAVVGTALIKKYPKLESEFVYGDYKVYDVRKMIA |
| | | KSEQEIGKATAKYFFYSNIMNFFKTEITLANGEIRKRPLIETNG |
| | | ETGEIVWDKGRDFATVRKVLSMPQVNIVKKTEVQTGGFSKES |
| | | ILPKRNSDKLIARKKDWDPKKYGGFDSPTVAYSVLVVAKVEK |
| | | GKSKKLKSVKELLGITIMERSSFEKNPIDFLEAKGYKEVKKDLI |
| | | IKLPKYSLFELENGRKRMLASAGELQKGNELALPSKYVNFLY |
| | | LASHYEKLKGSPEDNEQKQLFVEQHKHYLDEIIEQISEFSKRVI |
| | | LADANLDKVLSAYNKHRDKPIREQAENIIHLFTLTNLGAPAAF |
| | | KYFDTTIDRKRYTSTKEVLDATLIHQSITGLYETRIDLSQLGGD |
| | | SPKKKRKVGVDGSSGSETPGTSESATPESTGNKKLEAVGTGIE |
| | | PKAMSQGLVTFGDVAVDFSQEEWEWLNPIQRNLYRKVMLEN |
| | | YRNLASLGLCVSKPDVISSLEQGKEPWSADYKDDDDKAPKKK |
| | | RKVPKKKRKV |
| 662 | fusion protein | MGTMPKKKRKVPKKKRKVYNHDQEFDPPKVYPPVPAEKRKP |
| | (Configuration 11) | IRVLSLFDGIATGLLVLKDLGIQVDRYIASEVCEDSITVGMVRH |
| | | QGKIMYVGDVRSVTQKHIQEWGPFDLVIGGSPCNDLSIVNPA |
| | | RKGLYEGTGRLFFEFYRLLHDARPKEGDDRPFFWLFENVVAM |
| | | GVSDKRDISRFLESNPVMIDAKEVSAAHRARYFWGNLPGMNR |
| | | PLASTVNDKLELQECLEHGRIAKFSKVRTITTRSNSIKQGKDQ |
| | | HFPVFMNEKEDILWCTEMERVFGFPVHYTDVSNMSRLARQRL |
| | | LGRSWSVPVIRHLFAPLKEYFACVSSGNSNANSRGPSFSSGLV |
| | | PLSLRGSHMAAIPALDPEAEPSMDVILVGSSELSSSVSPGTGRD |
| | | LIAYEVKANQRNIEDICICCGSLQVHTQHPLFEGGICAPCKDKF |
| | | LDALFLYDDDGYQSYCSICCSGETLLICGNPDCTRCYCFECVD |
| | | SLVGPGTSGKVHAMSNWVCYLCLPSSRSGLLQRRRKWRSQL |
| | | KAFYDRESENPLEMFETVPVWRRQPVRVLSLFEDIKKELTSLG |
| | | FLESGSDPGQLKHVVDVTDTVRKDVEEWGPFDLVYGATPPLG |
| | | HTCDRPPSWYLFQFHRLLQYARPKPGSPRPFFWMFVDNLVLN |
| | | KEDLDVASRFLEMEPVTIPDVHGGSLQNAVRVWSNIPAIRSRH |
| | | WALVSEEELSLLAQNKQSSKLAAKWPTKLVKNCFLPLREYFK |
| | | YFSTELTSSLGGPSSGAPPPSGGSPAGSPTSTEEGTSESATPESG |
| | | PGTSTEPSEGSAPGSPAGSPTSTEEGTSTEPSEGSAPGTSTEPSE |
| | | LEDKKYSIGLAIGTNSVGWAVITDEYKVPSKKFKVLGNTDRH |
| | | SIKKNLIGALLFDSGETAEATRLKRTARRRYTRRKNRICYLQEI |
| | | FSNEMAKVDDSFFHRLEESFLVEEDKKHERHPIFGNIVDEVAY |
| | | HEKYPTIYHLRKKLVDSTDKADLRLIYLALAHMIKFRGHFLIE |
| | | GDLNPDNSDVDKLFIQLVQTYNQLFEENPINASGVDAKAILSA |
| | | RLSKSRRLENLIAQLPGEKKNGLFGNLIALSLGLTPNFKSNFDL |
| | | AEDAKLQLSKDTYDDDLDNLLAQIGDQYADLFLAAKNLSDAI |
| | | LLSDILRVNTEITKAPLSASMIKRYDEHHQDLTLLKALVRQQL |
| | | PEKYKEIFFDQSKNGYAGYIDGGASQEEFYKFIKPILEKMDGT |
| | | EELLVKLNREDLLRKQRTFDNGSIPHQIHLGELHAILRRQEDF |
| | | YPFLKDNREKIEKILTFRIPYYVGPLARGNSRFAWMTRKSEETI |
| | | TPWNFEEVVDKGASAQSFIERMTNFDKNLPNEKVLPKHSLLY |
| | | EYFTVYNELTKVKYVTEGMRKPAFLSGEQKKAIVDLLFKTNR |
| | | KVTVKQLKEDYFKKIECFDSVEISGVEDRFNASLGTYHDLLKII |
| | | KDKDFLDNEENEDILEDIVLTLTLFEDREMIEERLKTYAHLFD |
| | | DKVMKQLKRRRYTGWGRLSRKLINGIRDKQSGKTILDFLKSD |
| | | GFANRNFMQLIHDDSLTFKEDIQKAQVSGQGDSLHEHIANLA |
| | | GSPAIKKGILQTVKVVDELVKVMGRHKPENIVIEMARENQTT |
| | | QKGQKNSRERMKRIEEGIKELGSQILKEHPVENTQLQNEKLYL |
| | | YYLQNGRDMYVDQELDINRLSDYDVDAIVPQSFLKDDSIDNK |
| | | VLTRSDKNRGKSDNVPSEEVVKKMKNYWRQLLNAKLITQRK |
| | | FDNLTKAERGGLSELDKAGFIKRQLVETRQITKHVAQILDSRM |
| | | NTKYDENDKLIREVKVITLKSKLVSDFRKDFQFYKVREINNYH |
| | | HAHDAYLNAVVGTALIKKYPKLESEFVYGDYKVYDVRKMIA |
| | | KSEQEIGKATAKYFFYSNIMNFFKTEITLANGEIRKRPLIETNG |
| | | ETGEIVWDKGRDFATVRKVLSMPQVNIVKKTEVQTGGFSKES |
| | | ILPKRNSDKLIARKKDWDPKKYGGFDSPTVAYSVLVVAKVEK |
| | | GKSKKLKSVKELLGITIMERSSFEKNPIDFLEAKGYKEVKKDLI |
| | | IKLPKYSLFELENGRKRMLASAGELQKGNELALPSKYVNFLY |
| | | LASHYEKLKGSPEDNEQKQLFVEQHKHYLDEIIEQISEFSKRVI |
| | | LADANLDKVLSAYNKHRDKPIREQAENIIHLFTLTNLGAPAAF |
| | | KYFDTTIDRKRYTSTKEVLDATLIHQSITGLYETRIDLSQLGGD |
| | | SPKKKRKVGVDGSSGSETPGTSESATPESTGDSVAFEDVAVNF |
| | | TLEEWALLDPSQKNLYRDVMRETFRNLASVGKQWEDQNIED |
| | | PFKIPRRNISHIPERLCESKEGGQGEESADYKDDDDKAPKKKR |
| | | KVPKKKRKV |
| 663 | fusion protein | MGTMPKKKRKVPKKKRKVYNHDQEFDPPKVYPPVPAEKRKP |
| | (Configuration 13) | IRVLSLFDGIATGLLVLKDLGIQVDRYIASEVCEDSITVGMVRH |
| | | QGKIMYVGDVRSVTQKHIQEWGPFDLVIGGSPCNDLSIVNPA |
| | | RKGLYEGTGRLFFEFYRLLHDARPKEGDDRPFFWLFENVVAM |
| | | GVSDKRDISRFLESNPVMIDAKEVSAAHRARYFWGNLPGMNR |
| | | PLASTVNDKLELQECLEHGRIAKFSKVRTITTRSNSIKQGKDQ |
| | | HFPVFMNEKEDILWCTEMERVFGFPVHYTDVSNMSRLARQRL |
| | | LGRSWSVPVIRHLFAPLKEYFACVSSGNSNANSRGPSFSSGLV |
| | | PLSLRGSHMAAIPALDPEAEPSMDVILVGSSELSSSVSPGTGRD |
| | | LIAYEVKANQRNIEDICICCGSLQVHTQHPLFEGGICAPCKDKF |
| | | LDALFLYDDDGYQSYCSICCSGETLLICGNPDCTRCYCFECVD |
| | | SLVGPGTSGKVHAMSNWVCYLCLPSSRSGLLQRRRKWRSQL |
| | | KAFYDRESENPLEMFETVPVWRRQPVRVLSLFEDIKKELTSLG |
| | | FLESGSDPGQLKHVVDVTDTVRKDVEEWGPFDLVYGATPPLG |
| | | HTCDRPPSWYLFQFHRLLQYARPKPGSPRPFFWMFVDNLVLN |
| | | KEDLDVASRFLEMEPVTIPDVHGGSLQNAVRVWSNIPAIRSRH |
| | | WALVSEEELSLLAQNKQSSKLAAKWPTKLVKNCFLPLREYFK |
| | | YFSTELTSSLGGPSSGAPPPSGGSPAGSPTSTEEGTSESATPESG |
| | | PGTSTEPSEGSAPGSPAGSPTSTEEGTSTEPSEGSAPGTSTEPSE |
| | | LEDKKYSIGLAIGTNSVGWAVITDEYKVPSKKFKVLGNTDRH |
| | | SIKKNLIGALLFDSGETAEATRLKRTARRRYTRRKNRICYLQEI |
| | | FSNEMAKVDDSFFHRLEESFLVEEDKKHERHPIFGNIVDEVAY |
| | | HEKYPTIYHLRKKLVDSTDKADLRLIYLALAHMIKFRGHFLIE |
| | | GDLNPDNSDVDKLFIQLVQTYNQLFEENPINASGVDAKAILSA |
| | | RLSKSRRLENLIAQLPGEKKNGLFGNLIALSLGLTPNFKSNFDL |
| | | AEDAKLQLSKDTYDDDLDNLLAQIGDQYADLFLAAKNLSDAI |
| | | LLSDILRVNTEITKAPLSASMIKRYDEHHQDLTLLKALVRQQL |
| | | PEKYKEIFFDQSKNGYAGYIDGGASQEEFYKFIKPILEKMDGT |
| | | EELLVKLNREDLLRKQRTFDNGSIPHQIHLGELHAILRRQEDF |
| | | YPFLKDNREKIEKILTFRIPYYVGPLARGNSRFAWMTRKSEETI |
| | | TPWNFEEVVDKGASAQSFIERMTNFDKNLPNEKVLPKHSLLY |
| | | EYFTVYNELTKVKYVTEGMRKPAFLSGEQKKAIVDLLFKTNR |
| | | KVTVKQLKEDYFKKIECFDSVEISGVEDRFNASLGTYHDLLKII |
| | | KDKDFLDNEENEDILEDIVLTLTLFEDREMIEERLKTYAHLFD |
| | | DKVMKQLKRRRYTGWGRLSRKLINGIRDKQSGKTILDFLKSD |
| | | GFANRNFMQLIHDDSLTFKEDIQKAQVSGQGDSLHEHIANLA |
| | | GSPAIKKGILQTVKVVDELVKVMGRHKPENIVIEMARENQTT |
| | | QKGQKNSRERMKRIEEGIKELGSQILKEHPVENTQLQNEKLYL |
| | | YYLQNGRDMYVDQELDINRLSDYDVDAIVPQSFLKDDSIDNK |
| | | VLTRSDKNRGKSDNVPSEEVVKKMKNYWRQLLNAKLITQRK |
| | | FDNLTKAERGGLSELDKAGFIKRQLVETRQITKHVAQILDSRM |
| | | NTKYDENDKLIREVKVITLKSKLVSDFRKDFQFYKVREINNYH |
| | | HAHDAYLNAVVGTALIKKYPKLESEFVYGDYKVYDVRKMIA |
| | | KSEQEIGKATAKYFFYSNIMNFFKTEITLANGEIRKRPLIETNG |
| | | ETGEIVWDKGRDFATVRKVLSMPQVNIVKKTEVQTGGFSKES |
| | | ILPKRNSDKLIARKKDWDPKKYGGFDSPTVAYSVLVVAKVEK |
| | | GKSKKLKSVKELLGITIMERSSFEKNPIDFLEAKGYKEVKKDLI |
| | | IKLPKYSLFELENGRKRMLASAGELQKGNELALPSKYVNFLY |
| | | LASHYEKLKGSPEDNEQKQLFVEQHKHYLDEIIEQISEFSKRVI |
| | | LADANLDKVLSAYNKHRDKPIREQAENIIHLFTLTNLGAPAAF |
| | | KYFDTTIDRKRYTSTKEVLDATLIHQSITGLYETRIDLSQLGGD |
| | | SPKKKRKVGVDGSSGSETPGTSESATPESTGMNNSQGRVTFE |
| | | DVTVNFTQGEWQRLNPEQRNLYRDVMLENYSNLVSVGQGET |
| | | TKPDVILRLEQGKEPWLEEEEVLGSGRAEKNGDIGGQIWKPK |
| | | DVKESLSADYKDDDDKAPKKKRKVPKKKRKV |
| 667 | HLA-E variant 1 | ATGAGCCGATCCGTGGCACTGGCTGTCCTGGCTCTGCTGTC |
| | VMAPRTLFL | TCTGAGTGGCCTGGAAGCAGTGATGGCCCCTAGAACACTGT |
| | (SEQ ID NO: 678) | TCCTGGGCGGAGGCGGCTCCGGAGGAGGAGGGTCTGGAGG |
| | | CGGGGGAAGTATCCAGCGGACTCCCAAGATTCAGGTCTAC |
| | | AGCAGACACCCTGCCGAAAACGGGAAATCCAACTTCCTGA |
| | | ATTGCTATGTGTCAGGCTTTCATCCCAGCGACATCGAGGTC |
| | | GATCTGCTGAAGAATGGCGAGCGGATTGAAAAAGTGGAGC |
| | | ACTCTGACCTGTCATTCAGCAAGGATTGGAGCTTTTACCTG |
| | | CTGTACTATACTGAGTTTACCCCAACAGAAAAAGACGAGT |
| | | ATGCCTGTAGGGTGAACCATGTCACCCTGAGTCAGCCCAAG |
| | | ATCGTGAAATGGGACCGCGATATGGGTGGAGGAGGCTCTG |
| | | GGGGAGGCGGCTCCGGAGGCGGGGGAAGTGGCGGGGGAG |
| | | GATCCGGTTCTCACTCCTTGAAGTATTTCCACACTTCCGTGT |
| | | CCCGGCCCGGCCGCGGGGAGCCCCGCTTCATCTCTGTGGGC |
| | | TACGTGGACGACACCCAGTTCGTGCGCTTCGACAACGACGC |
| | | CGCGAGTCCGAGGATGGTGCCGCGGGCGCCGTGGATGGAG |
| | | CAGGAGGGGTCAGAGTATTGGGACCGGGAGACACGGAGCG |
| | | CCAGGGACACCGCACAGATTTTCCGAGTGAATCTGCGGAC |
| | | GCTGCGCGGCTACTACAATCAGAGCGAGGCCGGGTCTCAC |
| | | ACCCTGCAGTGGATGCATGGCTGCGAGCTGGGGCCCGACG |
| | | GGCGCTTCCTCCGCGGGTATGAACAGTTCGCCTACGACGGC |
| | | AAGGATTATCTCACCCTGAATGAGGACCTGCGCTCCTGGAC |
| | | CGCGGTGGACACGGCGGCTCAGATCTCCGAGCAAAAGTCA |
| | | AATGATGCCTCTGAGGCGGAGCACCAGAGAGCCTACCTGG |
| | | AAGACACATGCGTGGAGTGGCTCCACAAATACCTGGAGAA |
| | | GGGGAAGGAGACGCTGCTTCACCTGGAGCCCCCAAAGACA |
| | | CACGTGACTCACCACCCCATCTCTGACCATGAGGCCACCCT |
| | | GAGGTGCTGGGCCCTGGGCTTCTACCCTGCGGAGATCACAC |
| | | TGACCTGGCAGCAGGATGGGGAGGGCCATACCCAGGACAC |
| | | GGAGCTCGTGGAGACCAGGCCTGCAGGGGATGGAACCTTC |
| | | CAGAAGTGGGCAGCTGTGGTGGTGCCTTCTGGAGAGGAGC |
| | | AGAGATACACGTGCCATGTGCAGCATGAGGGGCTACCCGA |
| | | GCCCGTCACCCTGAGATGGAAGCCGGCTTCCCAGCCCACCA |
| | | TCCCCATCGTGGGCATCATTGCTGGCCTGGTTCTCCTTGGAT |
| | | CTGTGGTCTCTGGAGCTGTGGTTGCTGCTGTGATATGGAGG |
| | | AAGAAGAGCTCAGGTGGAAAAGGAGGGAGCTACTCTAAGG |
| | | CTGAGTGGAGCGACAGTGCCCAGGGGTCTGAGTCTCACAG |
| | | CTTGTAA |
| 668 | HLA-E variant 2 | ATGAGCCGATCCGTGGCACTGGCTGTCCTGGCTCTGCTGTC |
| | VMAPRTLIL | TCTGAGTGGCCTGGAAGCAGTGATGGCCCCTAGAACACTG |
| | (SEQ ID NO: 674) | ATCCTGGGCGGAGGCGGCTCCGGAGGAGGAGGGTCTGGAG |
| | | GCGGGGGAAGTATCCAGCGGACTCCCAAGATTCAGGTCTA |
| | | CAGCAGACACCCTGCCGAAAACGGGAAATCCAACTTCCTG |
| | | AATTGCTATGTGTCAGGCTTTCATCCCAGCGACATCGAGGT |
| | | CGATCTGCTGAAGAATGGCGAGCGGATTGAAAAAGTGGAG |
| | | CACTCTGACCTGTCATTCAGCAAGGATTGGAGCTTTTACCT |
| | | GCTGTACTATACTGAGTTTACCCCAACAGAAAAAGACGAG |
| | | TATGCCTGTAGGGTGAACCATGTCACCCTGAGTCAGCCCAA |
| | | GATCGTGAAATGGGACCGCGATATGGGTGGAGGAGGCTCT |
| | | GGGGGAGGCGGCTCCGGAGGCGGGGGAAGTGGCGGGGGA |
| | | GGATCCGGTTCTCACTCCTTGAAGTATTTCCACACTTCCGTG |
| | | TCCCGGCCCGGCCGCGGGGAGCCCCGCTTCATCTCTGTGGG |
| | | CTACGTGGACGACACCCAGTTCGTGCGCTTCGACAACGACG |
| | | CCGCGAGTCCGAGGATGGTGCCGCGGGCGCCGTGGATGGA |
| | | GCAGGAGGGGTCAGAGTATTGGGACCGGGAGACACGGAGC |
| | | GCCAGGGACACCGCACAGATTTTCCGAGTGAATCTGCGGA |
| | | CGCTGCGCGGCTACTACAATCAGAGCGAGGCCGGGTCTCA |
| | | CACCCTGCAGTGGATGCATGGCTGCGAGCTGGGGCCCGAC |
| | | GGGCGCTTCCTCCGCGGGTATGAACAGTTCGCCTACGACGG |
| | | CAAGGATTATCTCACCCTGAATGAGGACCTGCGCTCCTGGA |
| | | CCGCGGTGGACACGGCGGCTCAGATCTCCGAGCAAAAGTC |
| | | AAATGATGCCTCTGAGGCGGAGCACCAGAGAGCCTACCTG |
| | | GAAGACACATGCGTGGAGTGGCTCCACAAATACCTGGAGA |
| | | AGGGGAAGGAGACGCTGCTTCACCTGGAGCCCCCAAAGAC |
| | | ACACGTGACTCACCACCCCATCTCTGACCATGAGGCCACCC |
| | | TGAGGTGCTGGGCCCTGGGCTTCTACCCTGCGGAGATCACA |
| | | CTGACCTGGCAGCAGGATGGGGAGGGCCATACCCAGGACA |
| | | CGGAGCTCGTGGAGACCAGGCCTGCAGGGGATGGAACCTT |
| | | CCAGAAGTGGGCAGCTGTGGTGGTGCCTTCTGGAGAGGAG |
| | | CAGAGATACACGTGCCATGTGCAGCATGAGGGGCTACCCG |
| | | AGCCCGTCACCCTGAGATGGAAGCCGGCTTCCCAGCCCACC |
| | | ATCCCCATCGTGGGCATCATTGCTGGCCTGGTTCTCCTTGG |
| | | ATCTGTGGTCTCTGGAGCTGTGGTTGCTGCTGTGATATGGA |
| | | GGAAGAAGAGCTCAGGTGGAAAAGGAGGGAGCTACTCTAA |
| | | GGCTGAGTGGAGCGACAGTGCCCAGGGGTCTGAGTCTCAC |
| | | AGCTTGTAA |
| 669 | HLA-E variant 3 | |
| | VMAPRTLLL | ATGAGCCGATCCGTGGCACTGGCTGTCCTGGCTCTGCTGTC |
| | (SEQ ID NO: 675) | TCTGAGTGGCCTGGAAGCAGTGATGGCCCCTAGAACACTG |
| | | CTCCTGGGCGGAGGCGGCTCCGGAGGAGGAGGGTCTGGAG |
| | | GCGGGGGAAGTATCCAGCGGACTCCCAAGATTCAGGTCTA |
| | | CAGCAGACACCCTGCCGAAAACGGGAAATCCAACTTCCTG |
| | | AATTGCTATGTGTCAGGCTTTCATCCCAGCGACATCGAGGT |
| | | CGATCTGCTGAAGAATGGCGAGCGGATTGAAAAAGTGGAG |
| | | CACTCTGACCTGTCATTCAGCAAGGATTGGAGCTTTTACCT |
| | | GCTGTACTATACTGAGTTTACCCCAACAGAAAAAGACGAG |
| | | TATGCCTGTAGGGTGAACCATGTCACCCTGAGTCAGCCCAA |
| | | GATCGTGAAATGGGACCGCGATATGGGTGGAGGAGGCTCT |
| | | GGGGGAGGCGGCTCCGGAGGCGGGGGAAGTGGCGGGGGA |
| | | GGATCCGGTTCTCACTCCTTGAAGTATTTCCACACTTCCGTG |
| | | TCCCGGCCCGGCCGCGGGGAGCCCCGCTTCATCTCTGTGGG |
| | | CTACGTGGACGACACCCAGTTCGTGCGCTTCGACAACGACG |
| | | CCGCGAGTCCGAGGATGGTGCCGCGGGCGCCGTGGATGGA |
| | | GCAGGAGGGGTCAGAGTATTGGGACCGGGAGACACGGAGC |
| | | GCCAGGGACACCGCACAGATTTTCCGAGTGAATCTGCGGA |
| | | CGCTGCGCGGCTACTACAATCAGAGCGAGGCCGGGTCTCA |
| | | CACCCTGCAGTGGATGCATGGCTGCGAGCTGGGGCCCGAC |
| | | GGGCGCTTCCTCCGCGGGTATGAACAGTTCGCCTACGACGG |
| | | CAAGGATTATCTCACCCTGAATGAGGACCTGCGCTCCTGGA |
| | | CCGCGGTGGACACGGCGGCTCAGATCTCCGAGCAAAAGTC |
| | | AAATGATGCCTCTGAGGCGGAGCACCAGAGAGCCTACCTG |
| | | GAAGACACATGCGTGGAGTGGCTCCACAAATACCTGGAGA |
| | | AGGGGAAGGAGACGCTGCTTCACCTGGAGCCCCCAAAGAC |
| | | ACACGTGACTCACCACCCCATCTCTGACCATGAGGCCACCC |
| | | TGAGGTGCTGGGCCCTGGGCTTCTACCCTGCGGAGATCACA |
| | | CTGACCTGGCAGCAGGATGGGGAGGGCCATACCCAGGACA |
| | | CGGAGCTCGTGGAGACCAGGCCTGCAGGGGATGGAACCTT |
| | | CCAGAAGTGGGCAGCTGTGGTGGTGCCTTCTGGAGAGGAG |
| | | CAGAGATACACGTGCCATGTGCAGCATGAGGGGCTACCCG |
| | | AGCCCGTCACCCTGAGATGGAAGCCGGCTTCCCAGCCCACC |
| | | ATCCCCATCGTGGGCATCATTGCTGGCCTGGTTCTCCTTGG |
| | | ATCTGTGGTCTCTGGAGCTGTGGTTGCTGCTGTGATATGGA |
| | | GGAAGAAGAGCTCAGGTGGAAAAGGAGGGAGCTACTCTAA |
| | | GGCTGAGTGGAGCGACAGTGCCCAGGGGTCTGAGTCTCAC |
| | | AGCTTGTAA |
| 670 | HLA-E variant 4 | ATGAGCCGATCCGTGGCACTGGCTGTCCTGGCTCTGCTGTC |
| | VMAPRTLVL | TCTGAGTGGCCTGGAAGCAGTGATGGCCCCTAGAACACTG |
| | (SEQ ID NO: 676) | GTGCTGGGCGGAGGCGGCTCCGGAGGAGGAGGGTCTGGAG |
| | | GCGGGGGAAGTATCCAGCGGACTCCCAAGATTCAGGTCTA |
| | | CAGCAGACACCCTGCCGAAAACGGGAAATCCAACTTCCTG |
| | | AATTGCTATGTGTCAGGCTTTCATCCCAGCGACATCGAGGT |
| | | CGATCTGCTGAAGAATGGCGAGCGGATTGAAAAAGTGGAG |
| | | CACTCTGACCTGTCATTCAGCAAGGATTGGAGCTTTTACCT |
| | | GCTGTACTATACTGAGTTTACCCCAACAGAAAAAGACGAG |
| | | TATGCCTGTAGGGTGAACCATGTCACCCTGAGTCAGCCCAA |
| | | GATCGTGAAATGGGACCGCGATATGGGTGGAGGAGGCTCT |
| | | GGGGGAGGCGGCTCCGGAGGCGGGGGAAGTGGCGGGGGA |
| | | GGATCCGGTTCTCACTCCTTGAAGTATTTCCACACTTCCGTG |
| | | TCCCGGCCCGGCCGCGGGGAGCCCCGCTTCATCTCTGTGGG |
| | | CTACGTGGACGACACCCAGTTCGTGCGCTTCGACAACGACG |
| | | CCGCGAGTCCGAGGATGGTGCCGCGGGCGCCGTGGATGGA |
| | | GCAGGAGGGGTCAGAGTATTGGGACCGGGAGACACGGAGC |
| | | GCCAGGGACACCGCACAGATTTTCCGAGTGAATCTGCGGA |
| | | CGCTGCGCGGCTACTACAATCAGAGCGAGGCCGGGTCTCA |
| | | CACCCTGCAGTGGATGCATGGCTGCGAGCTGGGGCCCGAC |
| | | GGGCGCTTCCTCCGCGGGTATGAACAGTTCGCCTACGACGG |
| | | CAAGGATTATCTCACCCTGAATGAGGACCTGCGCTCCTGGA |
| | | CCGCGGTGGACACGGCGGCTCAGATCTCCGAGCAAAAGTC |
| | | AAATGATGCCTCTGAGGCGGAGCACCAGAGAGCCTACCTG |
| | | GAAGACACATGCGTGGAGTGGCTCCACAAATACCTGGAGA |
| | | AGGGGAAGGAGACGCTGCTTCACCTGGAGCCCCCAAAGAC |
| | | ACACGTGACTCACCACCCCATCTCTGACCATGAGGCCACCC |
| | | TGAGGTGCTGGGCCCTGGGCTTCTACCCTGCGGAGATCACA |
| | | CTGACCTGGCAGCAGGATGGGGAGGGCCATACCCAGGACA |
| | | CGGAGCTCGTGGAGACCAGGCCTGCAGGGGATGGAACCTT |
| | | CCAGAAGTGGGCAGCTGTGGTGGTGCCTTCTGGAGAGGAG |
| | | CAGAGATACACGTGCCATGTGCAGCATGAGGGGCTACCCG |
| | | AGCCCGTCACCCTGAGATGGAAGCCGGCTTCCCAGCCCACC |
| | | ATCCCCATCGTGGGCATCATTGCTGGCCTGGTTCTCCTTGG |
| | | ATCTGTGGTCTCTGGAGCTGTGGTTGCTGCTGTGATATGGA |
| | | GGAAGAAGAGCTCAGGTGGAAAAGGAGGGAGCTACTCTAA |
| | | GGCTGAGTGGAGCGACAGTGCCCAGGGGTCTGAGTCTCAC |
| | | AGCTTGTAA |
| 671 | HLA-E variant 5 | ATGAGCCGATCCGTGGCACTGGCTGTCCTGGCTCTGCTGTC |
| | VMAPRTVLL | TCTGAGTGGCCTGGAAGCAGTGATGGCCCCTAGAACAGTG |
| | (SEQ ID NO: 677) | CTGCTGGGCGGAGGCGGCTCCGGAGGAGGAGGGTCTGGAG |
| | | GCGGGGGAAGTATCCAGCGGACTCCCAAGATTCAGGTCTA |
| | | CAGCAGACACCCTGCCGAAAACGGGAAATCCAACTTCCTG |
| | | AATTGCTATGTGTCAGGCTTTCATCCCAGCGACATCGAGGT |
| | | CGATCTGCTGAAGAATGGCGAGCGGATTGAAAAAGTGGAG |
| | | CACTCTGACCTGTCATTCAGCAAGGATTGGAGCTTTTACCT |
| | | GCTGTACTATACTGAGTTTACCCCAACAGAAAAAGACGAG |
| | | TATGCCTGTAGGGTGAACCATGTCACCCTGAGTCAGCCCAA |
| | | GATCGTGAAATGGGACCGCGATATGGGTGGAGGAGGCTCT |
| | | GGGGGAGGCGGCTCCGGAGGCGGGGGAAGTGGCGGGGGA |
| | | GGATCCGGTTCTCACTCCTTGAAGTATTTCCACACTTCCGTG |
| | | TCCCGGCCCGGCCGCGGGGAGCCCCGCTTCATCTCTGTGGG |
| | | CTACGTGGACGACACCCAGTTCGTGCGCTTCGACAACGACG |
| | | CCGCGAGTCCGAGGATGGTGCCGCGGGCGCCGTGGATGGA |
| | | GCAGGAGGGGTCAGAGTATTGGGACCGGGAGACACGGAGC |
| | | GCCAGGGACACCGCACAGATTTTCCGAGTGAATCTGCGGA |
| | | CGCTGCGCGGCTACTACAATCAGAGCGAGGCCGGGTCTCA |
| | | CACCCTGCAGTGGATGCATGGCTGCGAGCTGGGGCCCGAC |
| | | GGGCGCTTCCTCCGCGGGTATGAACAGTTCGCCTACGACGG |
| | | CAAGGATTATCTCACCCTGAATGAGGACCTGCGCTCCTGGA |
| | | CCGCGGTGGACACGGCGGCTCAGATCTCCGAGCAAAAGTC |
| | | AAATGATGCCTCTGAGGCGGAGCACCAGAGAGCCTACCTG |
| | | GAAGACACATGCGTGGAGTGGCTCCACAAATACCTGGAGA |
| | | AGGGGAAGGAGACGCTGCTTCACCTGGAGCCCCCAAAGAC |
| | | ACACGTGACTCACCACCCCATCTCTGACCATGAGGCCACCC |
| | | TGAGGTGCTGGGCCCTGGGCTTCTACCCTGCGGAGATCACA |
| | | CTGACCTGGCAGCAGGATGGGGAGGGCCATACCCAGGACA |
| | | CGGAGCTCGTGGAGACCAGGCCTGCAGGGGATGGAACCTT |
| | | CCAGAAGTGGGCAGCTGTGGTGGTGCCTTCTGGAGAGGAG |
| | | CAGAGATACACGTGCCATGTGCAGCATGAGGGGCTACCCG |
| | | AGCCCGTCACCCTGAGATGGAAGCCGGCTTCCCAGCCCACC |
| | | ATCCCCATCGTGGGCATCATTGCTGGCCTGGTTCTCCTTGG |
| | | ATCTGTGGTCTCTGGAGCTGTGGTTGCTGCTGTGATATGGA |
| | | GGAAGAAGAGCTCAGGTGGAAAAGGAGGGAGCTACTCTAA |
| | | GGCTGAGTGGAGCGACAGTGCCCAGGGGTCTGAGTCTCAC |
| | | AGCTTGTAA |
| 672 | HLA-E variant 6 | ATGAGCCGATCCGTGGCACTGGCTGTCCTGGCTCTGCTGTC |
| | VMPPRTLLL | TCTGAGTGGCCTGGAAGCAGTGATGCCCCCTAGAACACTGC |
| | (SEQ ID NO: | TGCTGGGCGGAGGCGGCTCCGGAGGAGGAGGGTCTGGAGG |
| | 2110) | CGGGGGAAGTATCCAGCGGACTCCCAAGATTCAGGTCTAC |
| | | AGCAGACACCCTGCCGAAAACGGGAAATCCAACTTCCTGA |
| | | ATTGCTATGTGTCAGGCTTTCATCCCAGCGACATCGAGGTC |
| | | GATCTGCTGAAGAATGGCGAGCGGATTGAAAAAGTGGAGC |
| | | ACTCTGACCTGTCATTCAGCAAGGATTGGAGCTTTTACCTG |
| | | CTGTACTATACTGAGTTTACCCCAACAGAAAAAGACGAGT |
| | | ATGCCTGTAGGGTGAACCATGTCACCCTGAGTCAGCCCAAG |
| | | ATCGTGAAATGGGACCGCGATATGGGTGGAGGAGGCTCTG |
| | | GGGGAGGCGGCTCCGGAGGCGGGGGAAGTGGCGGGGGAG |
| | | GATCCGGTTCTCACTCCTTGAAGTATTTCCACACTTCCGTGT |
| | | CCCGGCCCGGCCGCGGGGAGCCCCGCTTCATCTCTGTGGGC |
| | | TACGTGGACGACACCCAGTTCGTGCGCTTCGACAACGACGC |
| | | CGCGAGTCCGAGGATGGTGCCGCGGGCGCCGTGGATGGAG |
| | | CAGGAGGGGTCAGAGTATTGGGACCGGGAGACACGGAGCG |
| | | CCAGGGACACCGCACAGATTTTCCGAGTGAATCTGCGGAC |
| | | GCTGCGCGGCTACTACAATCAGAGCGAGGCCGGGTCTCAC |
| | | ACCCTGCAGTGGATGCATGGCTGCGAGCTGGGGCCCGACG |
| | | GGCGCTTCCTCCGCGGGTATGAACAGTTCGCCTACGACGGC |
| | | AAGGATTATCTCACCCTGAATGAGGACCTGCGCTCCTGGAC |
| | | CGCGGTGGACACGGCGGCTCAGATCTCCGAGCAAAAGTCA |
| | | AATGATGCCTCTGAGGCGGAGCACCAGAGAGCCTACCTGG |
| | | AAGACACATGCGTGGAGTGGCTCCACAAATACCTGGAGAA |
| | | GGGGAAGGAGACGCTGCTTCACCTGGAGCCCCCAAAGACA |
| | | CACGTGACTCACCACCCCATCTCTGACCATGAGGCCACCCT |
| | | GAGGTGCTGGGCCCTGGGCTTCTACCCTGCGGAGATCACAC |
| | | TGACCTGGCAGCAGGATGGGGAGGGCCATACCCAGGACAC |
| | | GGAGCTCGTGGAGACCAGGCCTGCAGGGGATGGAACCTTC |
| | | CAGAAGTGGGCAGCTGTGGTGGTGCCTTCTGGAGAGGAGC |
| | | AGAGATACACGTGCCATGTGCAGCATGAGGGGCTACCCGA |
| | | GCCCGTCACCCTGAGATGGAAGCCGGCTTCCCAGCCCACCA |
| | | TCCCCATCGTGGGCATCATTGCTGGCCTGGTTCTCCTTGGAT |
| | | CTGTGGTCTCTGGAGCTGTGGTTGCTGCTGTGATATGGAGG |
| | | AAGAAGAGCTCAGGTGGAAAAGGAGGGAGCTACTCTAAGG |
| | | CTGAGTGGAGCGACAGTGCCCAGGGGTCTGAGTCTCACAG |
| | | CTTGTAA |
| 2112 | Fusion Protein | MKRPAATKKAGQAKKKYNHDQEFDPPKVYPPVPAEKRKPIR |
| | Configuration 15 | VLSLFDGIATGLLVLKDLGIQVDRYIASEVCEDSITVGMVRHQ |
| | | GKIMYVGDVRSVTQKHIQEWGPFDLVIGGSPCNDLSIVNPAR |
| | | KGLYEGTGRLFFEFYRLLHDARPKEGDDRPFFWLFENVVAMG |
| | | VSDKRDISRFLESNPVMIDAKEVSAAHRARYFWGNLPGMNRP |
| | | LASTVNDKLELQECLEHGRIAKFSKVRTITTRSNSIKQGKDQH |
| | | FPVFMNEKEDILWCTEMERVFGFPVHYTDVSNMSRLARQRLL |
| | | GRSWSVPVIRHLFAPLKEYFACVSSGNSNANSRGPSFSSGLVP |
| | | LSLRGSHSPLEMYKTVPVWKREPVRVLSLFGDIKKELTTLGFL |
| | | ENGSDPGRLKHLDDVTNTVRRDVEEWGPFDLVYGSTPPLGH |
| | | ACDHPPGWYLFQFHRVLQYARPRPGSPQAFFWMFVDNLVLT |
| | | EDDRAVATRFLETDPVTIQDVCGRAVRNAVHVWSNIPAVKSR |
| | | HSALFSQEESFLRAQDRQRAKPPARGPAKLVKNCFLPLREYFK |
| | | YFSTEFTSSLGGPSSGAPPPSGGSPAGSPTSTEEGTSESATPESG |
| | | PGTSTEPSEGSAPGSPAGSPTSTEEGTSTEPSEGSAPGTSTEPSE |
| | | LEDKKYSIGLAIGTNSVGWAVITDEYKVPSKKFKVLGNTDRH |
| | | SIKKNLIGALLFDSGETAEATRLKRTARRRYTRRKNRICYLQEI |
| | | FSNEMAKVDDSFFHRLEESFLVEEDKKHERHPIFGNIVDEVAY |
| | | HEKYPTIYHLRKKLVDSTDKADLRLIYLALAHMIKFRGHFLIE |
| | | GDLNPDNSDVDKLFIQLVQTYNQLFEENPINASGVDAKAILSA |
| | | RLSKSRRLENLIAQLPGEKKNGLFGNLIALSLGLTPNFKSNFDL |
| | | AEDAKLQLSKDTYDDDLDNLLAQIGDQYADLFLAAKNLSDAI |
| | | LLSDILRVNTEITKAPLSASMIKRYDEHHQDLTLLKALVRQQL |
| | | PEKYKEIFFDQSKNGYAGYIDGGASQEEFYKFIKPILEKMDGT |
| | | EELLVKLNREDLLRKQRTFDNGSIPHQIHLGELHAILRRQEDF |
| | | YPFLKDNREKIEKILTFRIPYYVGPLARGNSRFAWMTRKSEETI |
| | | TPWNFEEVVDKGASAQSFIERMTNFDKNLPNEKVLPKHSLLY |
| | | EYFTVYNELTKVKYVTEGMRKPAFLSGEQKKAIVDLLFKTNR |
| | | KVTVKQLKEDYFKKIECFDSVEISGVEDRFNASLGTYHDLLKII |
| | | KDKDFLDNEENEDILEDIVLTLTLFEDREMIEERLKTYAHLFD |
| | | DKVMKQLKRRRYTGWGRLSRKLINGIRDKQSGKTILDFLKSD |
| | | GFANRNFMQLIHDDSLTFKEDIQKAQVSGQGDSLHEHIANLA |
| | | GSPAIKKGILQTVKVVDELVKVMGRHKPENIVIEMARENQTT |
| | | QKGQKNSRERMKRIEEGIKELGSQILKEHPVENTQLQNEKLYL |
| | | YYLQNGRDMYVDQELDINRLSDYDVDAIVPQSFLKDDSIDNK |
| | | VLTRSDKNRGKSDNVPSEEVVKKMKNYWRQLLNAKLITQRK |
| | | FDNLTKAERGGLSELDKAGFIKRQLVETRQITKHVAQILDSRM |
| | | NTKYDENDKLIREVKVITLKSKLVSDFRKDFQFYKVREINNYH |
| | | HAHDAYLNAVVGTALIKKYPKLESEFVYGDYKVYDVRKMIA |
| | | KSEQEIGKATAKYFFYSNIMNFFKTEITLANGEIRKRPLIETNG |
| | | ETGEIVWDKGRDFATVRKVLSMPQVNIVKKTEVQTGGFSKES |
| | | ILPKRNSDKLIARKKDWDPKKYGGFDSPTVAYSVLVVAKVEK |
| | | GKSKKLKSVKELLGITIMERSSFEKNPIDFLEAKGYKEVKKDLI |
| | | IKLPKYSLFELENGRKRMLASAGELQKGNELALPSKYVNFLY |
| | | LASHYEKLKGSPEDNEQKQLFVEQHKHYLDEIIEQISEFSKRVI |
| | | LADANLDKVLSAYNKHRDKPIREQAENIIHLFTLTNLGAPAAF |
| | | KYFDTTIDRKRYTSTKEVLDATLIHQSITGLYETRIDLSQLGGD |
| | | SPKKKRKVGVDGSSGSETPGTSESATPESTGMNNSQGRVTFE |
| | | DVTVNFTQGEWQRLNPEQRNLYRDVMLENYSNLVSVGQGET |
| | | TKPDVILRLEQGKEPWLEEEEVLGSGRAEKNGDIGGQIWKPK |
| | | DVKESLSAKRPAATKKAGQAKKK |
| 2113 | Fusion Protein | AGGGGCGCTCGAGCAGGTTCAGAAGGAGATCAAAAACCCC |
| | Configuration 15 | CAAGGATCAAACATGAAGAGACCTGCTGCCACCAAGAAGG |
| | DNA | CCGGCCAGGCCAAGAAAAAGTACAATCACGATCAGGAGTT |
| | | CGACCCCCCTAAGGTGTACCCACCAGTGCCTGCAGAGAAG |
| | | AGGAAGCCAATCCGGGTGCTGAGCCTGTTTGATGGCATCGC |
| | | CACCGGCCTGCTGGTGCTGAAGGATCTGGGCATCCAGGTG |
| | | GACCGGTACATCGCCTCCGAGGTGTGCGAGGATTCTATCAC |
| | | CGTGGGCATGGTGCGCCACCAGGGCAAGATCATGTATGTG |
| | | GGCGACGTGCGGTCCGTGACACAGAAGCACATCCAGGAGT |
| | | GGGGCCCATTCGATCTGGTGATCGGCGGCAGCCCCTGTAAT |
| | | GACCTGTCCATCGTGAACCCTGCAAGGAAGGGACTGTACG |
| | | AGGGAACCGGCCGGCTGTTCTTTGAGTTTTATAGACTGCTG |
| | | CACGACGCCAGGCCTAAGGAGGGCGACGATAGACCATTCT |
| | | TTTGGCTGTTCGAGAATGTGGTGGCTATGGGCGTGAGCGAT |
| | | AAGAGGGACATCTCCAGGTTTCTGGAGTCTAACCCCGTGAT |
| | | GATCGATGCAAAGGAGGTGTCCGCCGCACACAGAGCCAGG |
| | | TATTTCTGGGGCAATCTGCCAGGAATGAACAGGCCACTGGC |
| | | AAGCACCGTGAATGACAAGCTGGAGCTGCAGGAGTGCCTG |
| | | GAGCACGGAAGGATCGCCAAGTTTTCCAAGGTGCGCACAA |
| | | TCACCACACGGAGCAATTCCATCAAGCAGGGCAAGGATCA |
| | | GCACTTCCCCGTGTTCATGAACGAGAAGGAGGACATCCTGT |
| | | GGTGTACCGAGATGGAGAGAGTGTTCGGCTTTCCAGTGCAC |
| | | TACACAGACGTGTCTAACATGAGCAGGCTGGCAAGGCAGC |
| | | GGCTGCTGGGCAGATCTTGGAGCGTGCCCGTGATCAGGCA |
| | | CCTGTTCGCCCCTCTGAAGGAGTATTTTGCCTGCGTGAGCA |
| | | GCGGCAACTCCAATGCCAACAGCCGGGGCCCCTCTTTCAGC |
| | | TCCGGATTGGTGCCTCTGAGCCTGAGGGGCTCCCACAGTCC |
| | | CCTTGAGATGTATAAAACTGTGCCTGTGTGGAAGAGAGAG |
| | | CCAGTGCGGGTGCTGTCCCTTTTTGGTGACATCAAGAAAGA |
| | | GCTGACGACTTTGGGCTTTCTGGAAAACGGCTCTGACCCGG |
| | | GCCGACTGAAACATTTGGACGATGTCACCAATACGGTGAG |
| | | GAGGGACGTGGAAGAATGGGGCCCGTTCGACCTCGTGTAC |
| | | GGCTCCACGCCGCCCCTCGGCCACGCCTGTGACCATCCTCC |
| | | CGGGTGGTACCTGTTCCAGTTCCACCGTGTGCTTCAGTACG |
| | | CGAGGCCCAGGCCGGGCAGCCCGCAGGCCTTCTTCTGGAT |
| | | GTTTGTGGACAACCTGGTGCTGACCGAGGATGACCGGGCT |
| | | GTAGCCACTCGCTTCCTGGAGACTGACCCGGTGACCATCCA |
| | | GGACGTCTGTGGCAGAGCTGTCCGGAACGCCGTGCACGTG |
| | | TGGAGCAACATCCCGGCCGTGAAAAGCAGGCACTCGGCCC |
| | | TGTTTTCCCAGGAGGAATCATTCCTGCGGGCTCAGGACAGG |
| | | CAGAGAGCAAAGCCCCCCGCCCGGGGGCCAGCCAAGCTGG |
| | | TGAAGAATTGTTTTCTCCCCCTGAGAGAATATTTCAAGTAT |
| | | TTTTCAACAGAATTCACTTCCTCTTTGGGAGGACCCTCCTCT |
| | | GGCGCCCCACCACCTAGCGGCGGCTCCCCTGCCGGCTCTCC |
| | | AACCAGCACAGAGGAGGGCACCAGCGAGTCCGCCACACCA |
| | | GAGTCTGGACCTGGCACCAGCACAGAGCCATCCGAGGGCT |
| | | CTGCCCCAGGCTCTCCTGCAGGCAGCCCTACCTCCACCGAA |
| | | GAGGGCACCAGCACAGAGCCTTCTGAGGGCAGCGCCCCAG |
| | | GCACCTCTACAGAGCCAAGCGAGCTCGAGGACAAGAAGTA |
| | | CAGCATCGGCCTGGCCATCGGCACCAACTCTGTGGGCTGGG |
| | | CCGTGATCACCGACGAGTACAAGGTGCCCAGCAAGAAATT |
| | | CAAGGTGCTGGGCAACACCGACCGGCACAGCATCAAGAAG |
| | | AACCTGATCGGAGCCCTGCTGTTCGACAGCGGCGAAACAG |
| | | CCGAGGCCACCCGGCTGAAGAGAACCGCCAGAAGAAGATA |
| | | CACCAGACGGAAGAACCGGATCTGCTATCTGCAAGAGATC |
| | | TTCAGCAACGAGATGGCCAAGGTGGACGACAGCTTCTTCC |
| | | ACAGACTGGAAGAGTCCTTCCTGGTGGAAGAGGATAAGAA |
| | | GCACGAGCGGCACCCCATCTTCGGCAACATCGTGGACGAG |
| | | GTGGCCTACCACGAGAAGTACCCCACCATCTACCACCTGAG |
| | | AAAGAAACTGGTGGACAGCACCGACAAGGCCGACCTGCGG |
| | | CTGATCTATCTGGCCCTGGCCCACATGATCAAGTTCCGGGG |
| | | CCACTTCCTGATCGAGGGCGACCTGAACCCCGACAACAGC |
| | | GACGTGGACAAGCTGTTCATCCAGCTGGTGCAGACCTACA |
| | | ACCAGCTGTTCGAGGAAAACCCCATCAACGCCAGCGGCGT |
| | | GGACGCCAAGGCCATCCTGTCTGCCAGACTGAGCAAGAGC |
| | | AGACGGCTGGAAAATCTGATCGCCCAGCTGCCCGGCGAGA |
| | | AGAAGAATGGCCTGTTCGGCAACCTGATTGCCCTGAGCCTG |
| | | GGCCTGACCCCCAACTTCAAGAGCAACTTCGACCTGGCCGA |
| | | GGATGCCAAACTGCAGCTGAGCAAGGACACCTACGACGAC |
| | | GACCTGGACAACCTGCTGGCCCAGATCGGCGACCAGTACG |
| | | CCGACCTGTTTCTGGCCGCCAAGAACCTGTCCGACGCCATC |
| | | CTGCTGAGCGACATCCTGAGAGTGAACACCGAGATCACCA |
| | | AGGCCCCCCTGAGCGCCTCTATGATCAAGAGATACGACGA |
| | | GCACCACCAGGACCTGACCCTGCTGAAAGCTCTCGTGCGGC |
| | | AGCAGCTGCCTGAGAAGTACAAAGAGATTTTCTTCGACCA |
| | | GAGCAAGAACGGCTACGCCGGCTACATTGACGGCGGAGCC |
| | | AGCCAGGAAGAGTTCTACAAGTTCATCAAGCCCATCCTGG |
| | | AAAAGATGGACGGCACCGAGGAACTGCTCGTGAAGCTGAA |
| | | CAGAGAGGACCTGCTGCGGAAGCAGCGGACCTTCGACAAC |
| | | GGCAGCATCCCCCACCAGATCCACCTGGGAGAGCTGCACG |
| | | CCATTCTGCGGCGGCAGGAAGATTTTTACCCATTCCTGAAG |
| | | GACAACCGGGAAAAGATCGAGAAGATCCTGACCTTCCGCA |
| | | TCCCCTACTACGTGGGCCCTCTGGCCAGGGGAAACAGCAG |
| | | ATTCGCCTGGATGACCAGAAAGAGCGAGGAAACCATCACC |
| | | CCCTGGAACTTCGAGGAAGTGGTGGACAAGGGCGCTTCCG |
| | | CCCAGAGCTTCATCGAGCGGATGACCAACTTCGATAAGAA |
| | | CCTGCCCAACGAGAAGGTGCTGCCCAAGCACAGCCTGCTG |
| | | TACGAGTACTTCACCGTGTATAACGAGCTGACCAAAGTGA |
| | | AATACGTGACCGAGGGAATGAGAAAGCCCGCCTTCCTGAG |
| | | CGGCGAGCAGAAAAAGGCCATCGTGGACCTGCTGTTCAAG |
| | | ACCAACCGGAAAGTGACCGTGAAGCAGCTGAAAGAGGACT |
| | | ACTTCAAGAAAATCGAGTGCTTCGACTCCGTGGAAATCTCC |
| | | GGCGTGGAAGATCGGTTCAACGCCTCCCTGGGCACATACC |
| | | ACGATCTGCTGAAAATTATCAAGGACAAGGACTTCCTGGA |
| | | CAATGAGGAAAACGAGGACATTCTGGAAGATATCGTGCTG |
| | | ACCCTGACACTGTTTGAGGACAGAGAGATGATCGAGGAAC |
| | | GGCTGAAAACCTATGCCCACCTGTTCGACGACAAAGTGAT |
| | | GAAGCAGCTGAAGCGGCGGAGATACACCGGCTGGGGCAGG |
| | | CTGAGCCGGAAGCTGATCAACGGCATCCGGGACAAGCAGT |
| | | CCGGCAAGACAATCCTGGATTTCCTGAAGTCCGACGGCTTC |
| | | GCCAACAGAAACTTCATGCAGCTGATCCACGACGACAGCC |
| | | TGACCTTTAAAGAGGACATCCAGAAAGCCCAGGTGTCCGG |
| | | CCAGGGCGATAGCCTGCACGAGCACATTGCCAATCTGGCC |
| | | GGCAGCCCCGCCATTAAGAAGGGCATCCTGCAGACAGTGA |
| | | AGGTGGTGGACGAGCTCGTGAAAGTGATGGGCCGGCACAA |
| | | GCCCGAGAACATCGTGATCGAAATGGCCAGAGAGAACCAG |
| | | ACCACCCAGAAGGGACAGAAGAACAGCCGCGAGAGAATG |
| | | AAGCGGATCGAAGAGGGCATCAAAGAGCTGGGCAGCCAG |
| | | ATCCTGAAAGAACACCCCGTGGAAAACACCCAGCTGCAGA |
| | | ACGAGAAGCTGTACCTGTACTACCTGCAGAATGGGGGGA |
| | | TATGTACGTGGACCAGGAACTGGACATCAACCGGCTGTCC |
| | | GACTACGATGTGGACGCCATCGTGCCTCAGAGCTTTCTGAA |
| | | GGACGACTCCATCGACAACAAGGTGCTGACCAGAAGCGAC |
| | | AAGAACCGGGGCAAGAGCGACAACGTGCCCTCCGAAGAGG |
| | | TCGTGAAGAAGATGAAGAACTACTGGCGGCAGCTGCTGAA |
| | | CGCCAAGCTGATTACCCAGAGAAAGTTCGACAATCTGACC |
| | | AAGGCCGAGAGAGGCGGCCTGAGCGAACTGGATAAGGCCG |
| | | GCTTCATCAAGAGACAGCTGGTGGAAACCCGGCAGATCAC |
| | | AAAGCACGTGGCACAGATCCTGGACTCCCGGATGAACACT |
| | | AAGTACGACGAGAATGACAAGCTGATCCGGGAAGTGAAAG |
| | | TGATCACCCTGAAGTCCAAGCTGGTGTCCGATTTCCGGAAG |
| | | GATTTCCAGTTTTACAAAGTGCGCGAGATCAACAACTACCA |
| | | CCACGCCCACGACGCCTACCTGAACGCCGTCGTGGGAACC |
| | | GCCCTGATCAAAAAGTACCCTAAGCTGGAAAGCGAGTTCG |
| | | TGTACGGCGACTACAAGGTGTACGACGTGCGGAAGATGAT |
| | | CGCCAAGAGCGAGCAGGAAATCGGCAAGGCTACCGCCAAG |
| | | TACTTCTTCTACAGCAACATCATGAACTTTTTCAAGACCGA |
| | | GATTACCCTGGCCAACGGCGAGATCCGGAAGCGGCCTCTG |
| | | ATCGAGACAAACGGCGAAACCGGGGAGATCGTGTGGGATA |
| | | AGGGCCGGGATTTTGCCACCGTGCGGAAAGTGCTGAGCAT |
| | | GCCCCAAGTGAATATCGTGAAAAAGACCGAGGTGCAGACA |
| | | GGCGGCTTCAGCAAAGAGTCTATCCTGCCCAAGAGGAACA |
| | | GCGATAAGCTGATCGCCAGAAAGAAGGACTGGGACCCTAA |
| | | GAAGTACGGCGGCTTCGACAGCCCCACCGTGGCCTATTCTG |
| | | TGCTGGTGGTGGCCAAAGTGGAAAAGGGCAAGTCCAAGAA |
| | | ACTGAAGAGTGTGAAAGAGCTGCTGGGGATCACCATCATG |
| | | GAAAGAAGCAGCTTCGAGAAGAATCCCATCGACTTTCTGG |
| | | AAGCCAAGGGCTACAAAGAAGTGAAAAAGGACCTGATCAT |
| | | CAAGCTGCCTAAGTACTCCCTGTTCGAGCTGGAAAACGGCC |
| | | GGAAGAGAATGCTGGCCTCTGCCGGCGAACTGCAGAAGGG |
| | | AAACGAACTGGCCCTGCCCTCCAAATATGTGAACTTCCTGT |
| | | ACCTGGCCAGCCACTATGAGAAGCTGAAGGGCTCCCCCGA |
| | | GGATAATGAGCAGAAACAGCTGTTTGTGGAACAGCACAAG |
| | | CACTACCTGGACGAGATCATCGAGCAGATCAGCGAGTTCTC |
| | | CAAGAGAGTGATCCTGGCCGACGCTAATCTGGACAAAGTG |
| | | CTGTCCGCCTACAACAAGCACCGGGATAAGCCCATCAGAG |
| | | AGCAGGCCGAGAATATCATCCACCTGTTTACCCTGACCAAT |
| | | CTGGGAGCCCCTGCCGCCTTCAAGTACTTTGACACCACCAT |
| | | CGACCGGAAGAGGTACACCAGCACCAAAGAGGTGCTGGAC |
| | | GCCACCCTGATCCACCAGAGCATCACCGGCCTGTACGAGA |
| | | CACGGATCGACCTGTCTCAGCTGGGAGGCGACAGCCCCAA |
| | | GAAGAAGAGAAAGGTGGGAGTCGACGGATCCAGCGGCTCC |
| | | GAGACCCCAGGCACATCTGAGAGCGCCACCCCTGAGTCCA |
| | | CCGGTATGAACAATTCACAGGGGAGAGTGACATTCGAAGA |
| | | CGTGACCGTGAACTTCACCCAGGGAGAATGGCAGCGCTTG |
| | | AACCCAGAACAAAGGAACCTCTATCGGGACGTGATGCTGG |
| | | AAAACTACTCAAATTTGGTGAGCGTTGGGCAGGGTGAGAC |
| | | CACTAAGCCTGACGTGATCCTGAGATTGGAACAGGGCAAG |
| | | GAGCCTTGGCTCGAGGAAGAGGAAGTCCTGGGCTCAGGGA |
| | | GGGCCGAGAAAAACGGTGATATAGGAGGCCAGATATGGAA |
| | | GCCTAAGGACGTCAAGGAGAGCCTGAGCGCTAAACGTCCG |
| | | GCAGCAACCAAAAAAGCAGGTCAGGCCAAGAAAAAATGA |
| | | GGATCCTGAGTCTAGAAAAGATATATATAGGATTGAAGAT |
| | | CTCTCAGTTAAGTCTACAAAAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAGAAGAGCGAATTCACTG |
| | | GCCGTCGTTTTACAACGTCGTGACTGGGAAAACCCTGGCGT |
| | | TACCCAACTTAATCGCCTTGCAGCACATCCCCCTTTCGCCA |
| | | GCTGGCGTAATAGCGAAGAGGCCCGCACCGATCGCCCTTC |
| | | CCAACAGTTGCGCAGCCTGAATGGCGAATGGCGCCTGATG |
| | | CGGTATTTTCTCCTTACGCATCTGTGCGGTATTTCACACCGC |
| | | ATATGGTGCACTCTCAGTACAATCTGCTCTGATGCCGCATA |
| | | GTTAAGCCAGCCCCGACACCCGCCAACACCCGCTGACGCG |
| | | CCCTGACGGGCTTGTCTGCTCCCGGCATCCGCTTACAGACA |
| | | AGCTGTGACCGTCTCCGGGAGCTGCATGTGTCAGAGGTTTT |
| | | CACCGTCATCACCGAAACGCGCGATGCAGCTCTGGCCCGTG |
| | | TCTCAAAATCTCTGATGTTACATTGCACAAGATAAAAATAT |
| | | ATCATCATGAACAATAAAACTGTCTGCTTACATAAACAGTA |
| | | ATACAAGGGGTGTTATGAGCCATATTCAACGGGAAACGTC |
| | | GAGGCCGCGATTAAATTCCAACATGGATGCTGATTTATATG |
| | | GGTATAAATGGGCTCGCGATAATGTCGGGCAATCAGGTGC |
| | | GACAATCTATCGCTTGTATGGGAAGCCCGATGCGCCAGAGT |
| | | TGTTTCTGAAACATGGCAAAGGTAGCGTTGCCAATGATGTT |
| | | ACAGATGAGATGGTCAGACTAAACTGGCTGACGGAATTTA |
| | | TGCCTCTTCCGACCATCAAGCATTTTATCCGTACTCCTGATG |
| | | ATGCATGGTTACTCACCACTGCGATCCCCGGAAAAACAGC |
| | | ATTCCAGGTATTAGAAGAATATCCTGATTCAGGTGAAAATA |
| | | TTGTTGATGCGCTGGCAGTGTTCCTGCGCCGGTTGCATTCG |
| | | ATTCCTGTTTGTAATTGTCCTTTTAACAGCGATCGCGTATTT |
| | | CGTCTCGCTCAGGCGCAATCACGAATGAATAACGGTTTGGT |
| | | TGATGCGAGTGATTTTGATGACGAGCGTAATGGCTGGCCTG |
| | | TTGAACAAGTCTGGAAAGAAATGCATAAACTTTTGCCATTC |
| | | TCACCGGATTCAGTCGTCACTCATGGTGATTTCTCACTTGAT |
| | | AACCTTATTTTTGACGAGGGGAAATTAATAGGTTGTATTGA |
| | | TGTTGGACGAGTCGGAATCGCAGACCGATACCAGGATCTT |
| | | GCCATCCTATGGAACTGCCTCGGTGAGTTTTCTCCTTCATTA |
| | | CAGAAACGGCTTTTTCAAAAATATGGTATTGATAATCCTGA |
| | | TATGAATAAATTGCAGTTTCATTTGATGCTCGATGAGTTTTT |
| | | CTAATCAGAATTGGTTAATTGGTTGTAACATTATTCAGATT |
| | | GGGCTTGATTTAAAACTTCATTTTTAATTTAAAAGGATCTA |
| | | GGTGAAGATCCTTTTTGATAATCTCATGACCAAAATCCCTT |
| | | AACGTGAGTTTTCGTTCCACTGAGCGTCAGACCCCGTAGAA |
| | | AAGATCAAAGGATCTTCTTGAGATCCTTTTTTTCTGCGCGT |
| | | AATCTGCTGCTTGCAAACAAAAAAACCACCGCTACCAGCG |
| | | GTGGTTTGTTTGCCGGATCAAGAGCTACCAACTCTTTTTCC |
| | | GAAGGTAACTGGCTTCAGCAGAGCGCAGATACCAAATACT |
| | | GTTCTTCTAGTGTAGCCGTAGTTAGGCCACCACTTCAAGAA |
| | | CTCTGTAGCACCGCCTACATACCTCGCTCTGCTAATCCTGTT |
| | | ACCAGTGGCTGCTGCCAGTGGCGATAAGTCGTGTCTTACCG |
| | | GGTTGGACTCAAGACGATAGTTACCGGATAAGGCGCAGCG |
| | | GTCGGGCTGAACGGGGGGTTCGTGCACACAGCCCAGCTTG |
| | | GAGCGAACGACCTACACCGAACTGAGATACCTACAGCGTG |
| | | AGCTATGAGAAAGCGCCACGCTTCCCGAAGGGAGAAAGGC |
| | | GGACAGGTATCCGGTAAGCGGCAGGGTCGGAACAGGAGAG |
| | | CGCACGAGGGAGCTTCCAGGGGGAAACGCCTGGTATCTTT |
| | | ATAGTCCTGTCGGGTTTCGCCACCTCTGACTTGAGCGTCGA |
| | | TTTTTGTGATGCTCGTCAGGGGGGCGGAGCCTATGGAAAAA |
| | | CGCCAGCAACGCGGCCTTTTTACGGTTCCTGGCCTTTTGCT |
| | | GGCCTTTTGCTCACATGTTCTTTCCTGCGTTATCCCCTGATT |
| | | CTGTGGATAACCGTATTACCGCCTTTGAGTGAGCTGATACC |
| | | GCTCGCCGCAGCCGAACGACCGAGCGCAGCGAGTCAGTGA |
| | | GCGAGGAAGCGGCCCAATACGCAAACCGCCTCTCCCCGCG |
| | | CGTTGGCCGATTCATTAATGCAGCTGGCACGACAGGTTTCC |
| | | CGACTGGAAAGCGGGCAGTGAGCGCAACGCAATTAATGTG |
| | | AGTTAGCTCACTCATTAGGCACCCCAGGCTTTACACTTTAT |
| | | GCTTCCGGCTCGTATGTTGTGTGGAATTGTGAGCGGATAAC |
| | | AATTTCACACAGGAAACAGCTATGACCATGATTACGCCAA |
| | | GCTTTAATACGACTCACTATA |
| 2114 | Fusion Protein | MKRPAATKKAGQAKKKYNHDQEFDPPKVYPPVPAEKRKPIR |
| | Configuration 16 | VLSLFDGIATGLLVLKDLGIQVDRYIASEVCEDSITVGMVRHQ |
| | | GKIMYVGDVRSVTQKHIQEWGPFDLVIGGSPCNDLSIVNPAR |
| | | KGLYEGTGRLFFEFYRLLHDARPKEGDDRPFFWLFENVVAMG |
| | | VSDKRDISRFLESNPVMIDAKEVSAAHRARYFWGNLPGMNRP |
| | | LASTVNDKLELQECLEHGRIAKFSKVRTITTRSNSIKQGKDQH |
| | | FPVFMNEKEDILWCTEMERVFGFPVHYTDVSNMSRLARQRLL |
| | | GRSWSVPVIRHLFAPLKEYFACVSSGNSNANSRGPSFSSGLVP |
| | | LSLRGSHSPMEIYKTVSAWKRQPVRVLSLFGNIDKELKSLGFL |
| | | EIGSDSEGGTLKYVEDVTNVVRRDVEKWGPFDLVYGSTNPLG |
| | | NSCDRCPGWYMFQFHRILQYARPRQDSQKPFFWIFMDNLLLT |
| | | EDDQVTTVRFLQTEAVTLQDVRGRVLQNAVRVWSNIPGLKS |
| | | KHSVLTPKEEQSLQAQVRTRSKLPTQVNPLVKTCLLPLREYFK |
| | | CFSQNSLPLGGPSSGAPPPSGGSPAGSPTSTEEGTSESATPESGP |
| | | GTSTEPSEGSAPGSPAGSPTSTEEGTSTEPSEGSAPGTSTEPSEL |
| | | EDKKYSIGLAIGTNSVGWAVITDEYKVPSKKFKVLGNTDRHSI |
| | | KKNLIGALLFDSGETAEATRLKRTARRRYTRRKNRICYLQEIF |
| | | SNEMAKVDDSFFHRLEESFLVEEDKKHERHPIFGNIVDEVAYH |
| | | EKYPTIYHLRKKLVDSTDKADLRLIYLALAHMIKFRGHFLIEG |
| | | DLNPDNSDVDKLFIQLVQTYNQLFEENPINASGVDAKAILSAR |
| | | LSKSRRLENLIAQLPGEKKNGLFGNLIALSLGLTPNFKSNFDLA |
| | | EDAKLQLSKDTYDDDLDNLLAQIGDQYADLFLAAKNLSDAIL |
| | | LSDILRVNTEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFYP |
| | | FLKDNREKIEKILTFRIPYYVGPLARGNSRFAWMTRKSEETITP |
| | | WNFEEVVDKGASAQSFIERMTNFDKNLPNEKVLPKHSLLYEY |
| | | FTVYNELTKVKYVTEGMRKPAFLSGEQKKAIVDLLFKTNRKV |
| | | TVKQLKEDYFKKIECFDSVEISGVEDRFNASLGTYHDLLKIIKD |
| | | KDFLDNEENEDILEDIVLTLTLFEDREMIEERLKTYAHLFDDK |
| | | VMKQLKRRRYTGWGRLSRKLINGIRDKQSGKTILDFLKSDGF |
| | | ANRNFMQLIHDDSLTFKEDIQKAQVSGQGDSLHEHIANLAGSP |
| | | AIKKGILQTVKVVDELVKVMGRHKPENIVIEMARENQTTQKG |
| | | QKNSRERMKRIEEGIKELGSQILKEHPVENTQLQNEKLYLYYL |
| | | QNGRDMYVDQELDINRLSDYDVDAIVPQSFLKDDSIDNKVLT |
| | | RSDKNRGKSDNVPSEEVVKKMKNYWRQLLNAKLITQRKFDN |
| | | LTKAERGGLSELDKAGFIKRQLVETRQITKHVAQILDSRMNTK |
| | | YDENDKLIREVKVITLKSKLVSDFRKDFQFYKVREINNYHHA |
| | | HDAYLNAVVGTALIKKYPKLESEFVYGDYKVYDVRKMIAKS |
| | | EQEIGKATAKYFFYSNIMNFFKTEITLANGEIRKRPLIETNGET |
| | | GEIVWDKGRDFATVRKVLSMPQVNIVKKTEVQTGGFSKESIL |
| | | PKRNSDKLIARKKDWDPKKYGGFDSPTVAYSVLVVAKVEKG |
| | | KSKKLKSVKELLGITIMERSSFEKNPIDFLEAKGYKEVKKDLII |
| | | KLPKYSLFELENGRKRMLASAGELQKGNELALPSKYVNFLYL |
| | | ASHYEKLKGSPEDNEQKQLFVEQHKHYLDEIIEQISEFSKRVIL |
| | | ADANLDKVLSAYNKHRDKPIREQAENIIHLFTLTNLGAPAAFK |
| | | YFDTTIDRKRYTSTKEVLDATLIHQSITGLYETRIDLSQLGGDS |
| | | PKKKRKVGVDGSSGSETPGTSESATPESTGMNNSQGRVTFED |
| | | VTVNFTQGEWQRLNPEQRNLYRDVMLENYSNLVSVGQGETT |
| | | KPDVILRLEQGKEPWLEEEEVLGSGRAEKNGDIGGQIWKPKD |
| | | VKESLSAAKRPAATKKAGQAKKK |
| 2115 | Fusion Protein | AGAAACTAGCGTAAATTCAAATATAGGTCAGGCTTCAACG |
| | Configuration 16 | TCAACAAATATGATGAAGAGACCTGCTGCCACCAAGAAGG |
| | DNA | CCGGCCAGGCCAAGAAAAAGTACAATCACGATCAGGAGTT |
| | | CGACCCCCCTAAGGTGTACCCACCAGTGCCTGCAGAGAAG |
| | | AGGAAGCCAATCCGGGTGCTGAGCCTGTTTGATGGCATCGC |
| | | CACCGGCCTGCTGGTGCTGAAGGATCTGGGCATCCAGGTG |
| | | GACCGGTACATCGCCTCCGAGGTGTGCGAGGATTCTATCAC |
| | | CGTGGGCATGGTGCGCCACCAGGGCAAGATCATGTATGTG |
| | | GGCGACGTGCGGTCCGTGACACAGAAGCACATCCAGGAGT |
| | | GGGGCCCATTCGATCTGGTGATCGGCGGCAGCCCCTGTAAT |
| | | GACCTGTCCATCGTGAACCCTGCAAGGAAGGGACTGTACG |
| | | AGGGAACCGGCCGGCTGTTCTTTGAGTTTTATAGACTGCTG |
| | | CACGACGCCAGGCCTAAGGAGGGCGACGATAGACCATTCT |
| | | TTTGGCTGTTCGAGAATGTGGTGGCTATGGGCGTGAGCGAT |
| | | AAGAGGGACATCTCCAGGTTTCTGGAGTCTAACCCCGTGAT |
| | | GATCGATGCAAAGGAGGTGTCCGCCGCACACAGAGCCAGG |
| | | TATTTCTGGGGCAATCTGCCAGGAATGAACAGGCCACTGGC |
| | | AAGCACCGTGAATGACAAGCTGGAGCTGCAGGAGTGCCTG |
| | | GAGCACGGAAGGATCGCCAAGTTTTCCAAGGTGCGCACAA |
| | | TCACCACACGGAGCAATTCCATCAAGCAGGGCAAGGATCA |
| | | GCACTTCCCCGTGTTCATGAACGAGAAGGAGGACATCCTGT |
| | | GGTGTACCGAGATGGAGAGAGTGTTCGGCTTTCCAGTGCAC |
| | | TACACAGACGTGTCTAACATGAGCAGGCTGGCAAGGCAGC |
| | | GGCTGCTGGGCAGATCTTGGAGCGTGCCCGTGATCAGGCA |
| | | CCTGTTCGCCCCTCTGAAGGAGTATTTTGCCTGCGTGAGCA |
| | | GCGGCAACTCCAATGCCAACAGCCGGGGCCCCTCTTTCAGC |
| | | TCCGGATTGGTGCCTCTGAGCCTGAGGGGCTCCCACAGCCC |
| | | TATGGAGATATACAAGACAGTGTCTGCATGGAAGAGACAG |
| | | CCAGTGAGGGTGCTGAGCCTTTTTGGGAATATTGATAAAGA |
| | | ACTAAAGAGTTTGGGCTTTTTGGAAATCGGTTCTGATTCTG |
| | | AGGGAGGAACACTGAAGTACGTGGAAGATGTCACGAATGT |
| | | CGTGAGGAGAGACGTGGAGAAATGGGGCCCCTTTGACCTG |
| | | GTGTATGGCTCGACGAATCCCCTAGGCAACTCTTGTGACCG |
| | | CTGTCCTGGCTGGTACATGTTCCAATTCCACCGGATCCTGC |
| | | AGTATGCGCGGCCTCGCCAAGACAGTCAGAAGCCCTTCTTC |
| | | TGGATATTTATGGACAATCTGCTGCTGACTGAGGATGATCA |
| | | AGTGACAACTGTCCGCTTCCTTCAGACAGAGGCTGTGACCC |
| | | TCCAGGATGTCCGTGGCAGAGTCCTCCAGAATGCTGTGAGG |
| | | GTATGGAGCAACATTCCAGGACTGAAGAGTAAGCACTCAG |
| | | TCCTGACGCCAAAGGAAGAACAGTCTCTGCAAGCCCAAGT |
| | | CAGAACCAGAAGCAAGCTGCCCACCCAGGTTAACCCCCTG |
| | | GTGAAGACCTGCCTTCTCCCCCTGAGAGAGTACTTCAAGTG |
| | | TTTTTCTCAGAATTCACTTCCTCTTGGAGGACCCTCCTCTGG |
| | | CGCCCCACCACCTAGCGGCGGCTCCCCTGCCGGCTCTCCAA |
| | | CCAGCACAGAGGAGGGCACCAGCGAGTCCGCCACACCAGA |
| | | GTCTGGACCTGGCACCAGCACAGAGCCATCCGAGGGCTCT |
| | | GCCCCAGGCTCTCCTGCAGGCAGCCCTACCTCCACCGAAGA |
| | | GGGCACCAGCACAGAGCCTTCTGAGGGCAGCGCCCCAGGC |
| | | ACCTCTACAGAGCCAAGCGAGCTCGAGGACAAGAAGTACA |
| | | GCATCGGCCTGGCCATCGGCACCAACTCTGTGGGCTGGGCC |
| | | GTGATCACCGACGAGTACAAGGTGCCCAGCAAGAAATTCA |
| | | AGGTGCTGGGCAACACCGACCGGCACAGCATCAAGAAGAA |
| | | CCTGATCGGAGCCCTGCTGTTCGACAGCGGCGAAACAGCC |
| | | GAGGCCACCCGGCTGAAGAGAACCGCCAGAAGAAGATACA |
| | | CCAGACGGAAGAACCGGATCTGCTATCTGCAAGAGATCTT |
| | | CAGCAACGAGATGGCCAAGGTGGACGACAGCTTCTTCCAC |
| | | AGACTGGAAGAGTCCTTCCTGGTGGAAGAGGATAAGAAGC |
| | | ACGAGCGGCACCCCATCTTCGGCAACATCGTGGACGAGGT |
| | | GGCCTACCACGAGAAGTACCCCACCATCTACCACCTGAGA |
| | | AAGAAACTGGTGGACAGCACCGACAAGGCCGACCTGCGGC |
| | | TGATCTATCTGGCCCTGGCCCACATGATCAAGTTCCGGGGC |
| | | CACTTCCTGATCGAGGGCGACCTGAACCCCGACAACAGCG |
| | | ACGTGGACAAGCTGTTCATCCAGCTGGTGCAGACCTACAAC |
| | | CAGCTGTTCGAGGAAAACCCCATCAACGCCAGCGGCGTGG |
| | | ACGCCAAGGCCATCCTGTCTGCCAGACTGAGCAAGAGCAG |
| | | ACGGCTGGAAAATCTGATCGCCCAGCTGCCCGGCGAGAAG |
| | | AAGAATGGCCTGTTCGGCAACCTGATTGCCCTGAGCCTGGG |
| | | CCTGACCCCCAACTTCAAGAGCAACTTCGACCTGGCCGAGG |
| | | ATGCCAAACTGCAGCTGAGCAAGGACACCTACGACGACGA |
| | | CCTGGACAACCTGCTGGCCCAGATCGGCGACCAGTACGCC |
| | | GACCTGTTTCTGGCCGCCAAGAACCTGTCCGACGCCATCCT |
| | | GCTGAGCGACATCCTGAGAGTGAACACCGAGATCACCAAG |
| | | GCCCCCCTGAGCGCCTCTATGATCAAGAGATACGACGAGC |
| | | ACCACCAGGACCTGACCCTGCTGAAAGCTCTCGTGCGGCA |
| | | GCAGCTGCCTGAGAAGTACAAAGAGATTTTCTTCGACCAG |
| | | AGCAAGAACGGCTACGCCGGCTACATTGACGGCGGAGCCA |
| | | GCCAGGAAGAGTTCTACAAGTTCATCAAGCCCATCCTGGA |
| | | AAAGATGGACGGCACCGAGGAACTGCTCGTGAAGCTGAAC |
| | | AGAGAGGACCTGCTGCGGAAGCAGCGGACCTTCGACAACG |
| | | GCAGCATCCCCCACCAGATCCACCTGGGAGAGCTGCACGC |
| | | CATTCTGCGGCGGCAGGAAGATTTTTACCCATTCCTGAAGG |
| | | ACAACCGGGAAAAGATCGAGAAGATCCTGACCTTCCGCAT |
| | | CCCCTACTACGTGGGCCCTCTGGCCAGGGGAAACAGCAGA |
| | | TTCGCCTGGATGACCAGAAAGAGCGAGGAAACCATCACCC |
| | | CCTGGAACTTCGAGGAAGTGGTGGACAAGGGCGCTTCCGC |
| | | CCAGAGCTTCATCGAGCGGATGACCAACTTCGATAAGAAC |
| | | CTGCCCAACGAGAAGGTGCTGCCCAAGCACAGCCTGCTGT |
| | | ACGAGTACTTCACCGTGTATAACGAGCTGACCAAAGTGAA |
| | | ATACGTGACCGAGGGAATGAGAAAGCCCGCCTTCCTGAGC |
| | | GGCGAGCAGAAAAAGGCCATCGTGGACCTGCTGTTCAAGA |
| | | CCAACCGGAAAGTGACCGTGAAGCAGCTGAAAGAGGACTA |
| | | CTTCAAGAAAATCGAGTGCTTCGACTCCGTGGAAATCTCCG |
| | | GCGTGGAAGATCGGTTCAACGCCTCCCTGGGCACATACCAC |
| | | GATCTGCTGAAAATTATCAAGGACAAGGACTTCCTGGACA |
| | | ATGAGGAAAACGAGGACATTCTGGAAGATATCGTGCTGAC |
| | | CCTGACACTGTTTGAGGACAGAGAGATGATCGAGGAACGG |
| | | CTGAAAACCTATGCCCACCTGTTCGACGACAAAGTGATGA |
| | | AGCAGCTGAAGCGGCGGAGATACACCGGCTGGGGCAGGCT |
| | | GAGCCGGAAGCTGATCAACGGCATCCGGGACAAGCAGTCC |
| | | GGCAAGACAATCCTGGATTTCCTGAAGTCCGACGGCTTCGC |
| | | CAACAGAAACTTCATGCAGCTGATCCACGACGACAGCCTG |
| | | ACCTTTAAAGAGGACATCCAGAAAGCCCAGGTGTCCGGCC |
| | | AGGGCGATAGCCTGCACGAGCACATTGCCAATCTGGCCGG |
| | | CAGCCCCGCCATTAAGAAGGGCATCCTGCAGACAGTGAAG |
| | | GTGGTGGACGAGCTCGTGAAAGTGATGGGCCGGCACAAGC |
| | | CCGAGAACATCGTGATCGAAATGGCCAGAGAGAACCAGAC |
| | | CACCCAGAAGGGACAGAAGAACAGCCGCGAGAGAATGAA |
| | | GCGGATCGAAGAGGGCATCAAAGAGCTGGGCAGCCAGATC |
| | | CTGAAAGAACACCCCGTGGAAAACACCCAGCTGCAGAACG |
| | | AGAAGCTGTACCTGTACTACCTGCAGAATGGGCGGGATAT |
| | | GTACGTGGACCAGGAACTGGACATCAACCGGCTGTCCGAC |
| | | TACGATGTGGACGCCATCGTGCCTCAGAGCTTTCTGAAGGA |
| | | CGACTCCATCGACAACAAGGTGCTGACCAGAAGCGACAAG |
| | | AACCGGGGCAAGAGCGACAACGTGCCCTCCGAAGAGGTCG |
| | | TGAAGAAGATGAAGAACTACTGGCGGCAGCTGCTGAACGC |
| | | CAAGCTGATTACCCAGAGAAAGTTCGACAATCTGACCAAG |
| | | GCCGAGAGAGGCGGCCTGAGCGAACTGGATAAGGCCGGCT |
| | | TCATCAAGAGACAGCTGGTGGAAACCCGGCAGATCACAAA |
| | | GCACGTGGCACAGATCCTGGACTCCCGGATGAACACTAAG |
| | | TACGACGAGAATGACAAGCTGATCCGGGAAGTGAAAGTGA |
| | | TCACCCTGAAGTCCAAGCTGGTGTCCGATTTCCGGAAGGAT |
| | | TTCCAGTTTTACAAAGTGCGCGAGATCAACAACTACCACCA |
| | | CGCCCACGACGCCTACCTGAACGCCGTCGTGGGAACCGCC |
| | | CTGATCAAAAAGTACCCTAAGCTGGAAAGCGAGTTCGTGT |
| | | ACGGCGACTACAAGGTGTACGACGTGCGGAAGATGATCGC |
| | | CAAGAGCGAGCAGGAAATCGGCAAGGCTACCGCCAAGTAC |
| | | TTCTTCTACAGCAACATCATGAACTTTTTCAAGACCGAGAT |
| | | TACCCTGGCCAACGGCGAGATCCGGAAGCGGCCTCTGATC |
| | | GAGACAAACGGCGAAACCGGGGAGATCGTGTGGGATAAG |
| | | GGCCGGGATTTTGCCACCGTGCGGAAAGTGCTGAGCATGC |
| | | CCCAAGTGAATATCGTGAAAAAGACCGAGGTGCAGACAGG |
| | | CGGCTTCAGCAAAGAGTCTATCCTGCCCAAGAGGAACAGC |
| | | GATAAGCTGATCGCCAGAAAGAAGGACTGGGACCCTAAGA |
| | | AGTACGGCGGCTTCGACAGCCCCACCGTGGCCTATTCTGTG |
| | | CTGGTGGTGGCCAAAGTGGAAAAGGGCAAGTCCAAGAAAC |
| | | TGAAGAGTGTGAAAGAGCTGCTGGGGATCACCATCATGGA |
| | | AAGAAGCAGCTTCGAGAAGAATCCCATCGACTTTCTGGAA |
| | | GCCAAGGGCTACAAAGAAGTGAAAAAGGACCTGATCATCA |
| | | AGCTGCCTAAGTACTCCCTGTTCGAGCTGGAAAACGGCCGG |
| | | AAGAGAATGCTGGCCTCTGCCGGCGAACTGCAGAAGGGAA |
| | | ACGAACTGGCCCTGCCCTCCAAATATGTGAACTTCCTGTAC |
| | | CTGGCCAGCCACTATGAGAAGCTGAAGGGCTCCCCCGAGG |
| | | ATAATGAGCAGAAACAGCTGTTTGTGGAACAGCACAAGCA |
| | | CTACCTGGACGAGATCATCGAGCAGATCAGCGAGTTCTCCA |
| | | AGAGAGTGATCCTGGCCGACGCTAATCTGGACAAAGTGCT |
| | | GTCCGCCTACAACAAGCACCGGGATAAGCCCATCAGAGAG |
| | | CAGGCCGAGAATATCATCCACCTGTTTACCCTGACCAATCT |
| | | GGGAGCCCCTGCCGCCTTCAAGTACTTTGACACCACCATCG |
| | | ACCGGAAGAGGTACACCAGCACCAAAGAGGTGCTGGACGC |
| | | CACCCTGATCCACCAGAGCATCACCGGCCTGTACGAGACA |
| | | CGGATCGACCTGTCTCAGCTGGGAGGCGACAGCCCCAAGA |
| | | AGAAGAGAAAGGTGGGAGTCGACGGATCCAGCGGCTCCGA |
| | | GACCCCAGGCACATCTGAGAGCGCCACCCCTGAGTCCACC |
| | | GGTATGAACAATTCACAGGGGAGAGTGACATTCGAAGACG |
| | | TGACCGTGAACTTCACCCAGGGAGAATGGCAGCGCTTGAA |
| | | CCCAGAACAAAGGAACCTCTATCGGGACGTGATGCTGGAA |
| | | AACTACTCAAATTTGGTGAGCGTTGGGCAGGGTGAGACCA |
| | | CTAAGCCTGACGTGATCCTGAGATTGGAACAGGGCAAGGA |
| | | GCCTTGGCTCGAGGAAGAGGAAGTCCTGGGCTCAGGGAGG |
| | | GCCGAGAAAAACGGTGATATAGGAGGCCAGATATGGAAGC |
| | | CTAAGGACGTCAAGGAGAGCCTGAGCGCTGCTAAACGTCC |
| | | GGCAGCAACCAAAAAAGCAGGTCAGGCCAAGAAAAAATG |
| | | AGGATCCTGAGTCTAGAAAGAGCCTTCTGAGCCCAGCGAC |
| | | TTCTGAAGGGCCCCTTGCAAAGTAATAGGGCTTCTGCCTAA |
| | | GCCTCTCCCTCCAGCCAATAGGCAGCTTTCTTAACTATCCT |
| | | AACAAGCCTTGGACCAAATGGAAATAAAGCTTTTTGATGC |
| | | AGTGTTAATTAAAAAAAAAAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAAAAAAAAAAAAAAAAA |
| | | AAAAAAAAAAAAAAAAGAAGAGCGAATTCACTGGCCGTC |
| | | GTTTTACAACGTCGTGACTGGGAAAACCCTGGCGTTACCCA |
| | | ACTTAATCGCCTTGCAGCACATCCCCCTTTCGCCAGCTGGC |
| | | GTAATAGCGAAGAGGCCCGCACCGATCGCCCTTCCCAACA |
| | | GTTGCGCAGCCTGAATGGCGAATGGCGCCTGATGCGGTATT |
| | | TTCTCCTTACGCATCTGTGCGGTATTTCACACCGCATATGGT |
| | | GCACTCTCAGTACAATCTGCTCTGATGCCGCATAGTTAAGC |
| | | CAGCCCCGACACCCGCCAACACCCGCTGACGCGCCCTGAC |
| | | GGGCTTGTCTGCTCCCGGCATCCGCTTACAGACAAGCTGTG |
| | | ACCGTCTCCGGGAGCTGCATGTGTCAGAGGTTTTCACCGTC |
| | | ATCACCGAAACGCGCGATGCAGCTCTGGCCCGTGTCTCAAA |
| | | ATCTCTGATGTTACATTGCACAAGATAAAAATATATCATCA |
| | | TGAACAATAAAACTGTCTGCTTACATAAACAGTAATACAA |
| | | GGGGTGTTATGAGCCATATTCAACGGGAAACGTCGAGGCC |
| | | GCGATTAAATTCCAACATGGATGCTGATTTATATGGGTATA |
| | | AATGGGCTCGCGATAATGTCGGGCAATCAGGTGCGACAAT |
| | | CTATCGCTTGTATGGGAAGCCCGATGCGCCAGAGTTGTTTC |
| | | TGAAACATGGCAAAGGTAGCGTTGCCAATGATGTTACAGA |
| | | TGAGATGGTCAGACTAAACTGGCTGACGGAATTTATGCCTC |
| | | TTCCGACCATCAAGCATTTTATCCGTACTCCTGATGATGCA |
| | | TGGTTACTCACCACTGCGATCCCCGGAAAAACAGCATTCCA |
| | | GGTATTAGAAGAATATCCTGATTCAGGTGAAAATATTGTTG |
| | | ATGCGCTGGCAGTGTTCCTGCGCCGGTTGCATTCGATTCCT |
| | | GTTTGTAATTGTCCTTTTAACAGCGATCGCGTATTTCGTCTC |
| | | GCTCAGGCGCAATCACGAATGAATAACGGTTTGGTTGATGC |
| | | GAGTGATTTTGATGACGAGCGTAATGGCTGGCCTGTTGAAC |
| | | AAGTCTGGAAAGAAATGCATAAACTTTTGCCATTCTCACCG |
| | | GATTCAGTCGTCACTCATGGTGATTTCTCACTTGATAACCTT |
| | | ATTTTTGACGAGGGGAAATTAATAGGTTGTATTGATGTTGG |
| | | ACGAGTCGGAATCGCAGACCGATACCAGGATCTTGCCATC |
| | | CTATGGAACTGCCTCGGTGAGTTTTCTCCTTCATTACAGAA |
| | | ACGGCTTTTTCAAAAATATGGTATTGATAATCCTGATATGA |
| | | ATAAATTGCAGTTTCATTTGATGCTCGATGAGTTTTTCTAAT |
| | | CAGAATTGGTTAATTGGTTGTAACATTATTCAGATTGGGCT |
| | | TGATTTAAAACTTCATTTTTAATTTAAAAGGATCTAGGTGA |
| | | AGATCCTTTTTGATAATCTCATGACCAAAATCCCTTAACGT |
| | | GAGTTTTCGTTCCACTGAGCGTCAGACCCCGTAGAAAAGAT |
| | | CAAAGGATCTTCTTGAGATCCTTTTTTTCTGCGCGTAATCTG |
| | | CTGCTTGCAAACAAAAAAACCACCGCTACCAGCGGTGGTTT |
| | | GTTTGCCGGATCAAGAGCTACCAACTCTTTTTCCGAAGGTA |
| | | ACTGGCTTCAGCAGAGCGCAGATACCAAATACTGTTCTTCT |
| | | AGTGTAGCCGTAGTTAGGCCACCACTTCAAGAACTCTGTAG |
| | | CACCGCCTACATACCTCGCTCTGCTAATCCTGTTACCAGTG |
| | | GCTGCTGCCAGTGGCGATAAGTCGTGTCTTACCGGGTTGGA |
| | | CTCAAGACGATAGTTACCGGATAAGGCGCAGCGGTCGGGC |
| | | TGAACGGGGGGTTCGTGCACACAGCCCAGCTTGGAGCGAA |
| | | CGACCTACACCGAACTGAGATACCTACAGCGTGAGCTATG |
| | | AGAAAGCGCCACGCTTCCCGAAGGGAGAAAGGCGGACAG |
| | | GTATCCGGTAAGCGGCAGGGTCGGAACAGGAGAGCGCACG |
| | | AGGGAGCTTCCAGGGGGAAACGCCTGGTATCTTTATAGTCC |
| | | TGTCGGGTTTCGCCACCTCTGACTTGAGCGTCGATTTTTGTG |
| | | ATGCTCGTCAGGGGGGCGGAGCCTATGGAAAAACGCCAGC |
| | | AACGCGGCCTTTTTACGGTTCCTGGCCTTTTGCTGGCCTTTT |
| | | GCTCACATGTTCTTTCCTGCGTTATCCCCTGATTCTGTGGAT |
| | | AACCGTATTACCGCCTTTGAGTGAGCTGATACCGCTCGCCG |
| | | CAGCCGAACGACCGAGCGCAGCGAGTCAGTGAGCGAGGAA |
| | | GCGGCCCAATACGCAAACCGCCTCTCCCCGCGCGTTGGCCG |
| | | ATTCATTAATGCAGCTGGCACGACAGGTTTCCCGACTGGAA |
| | | AGCGGGCAGTGAGCGCAACGCAATTAATGTGAGTTAGCTC |
| | | ACTCATTAGGCACCCCAGGCTTTACACTTTATGCTTCCGGC |
| | | TCGTATGTTGTGTGGAATTGTGAGCGGATAACAATTTCACA |
| | | CAGGAAACAGCTATGACCATGATTACGCCAAGCTTTAATAC |
| | | GACTCACTATA |
| 2116 | Fusion Protein | MKRPAATKKAGQAKKKYNHDQEFDPPKVYPPVPAEKRKPIR |
| | Configuration 17 | VLSLFDGIATGLLVLKDLGIQVDRYIASEVCEDSITVGMVRHQ |
| | | GKIMYVGDVRSVTQKHIQEWGPFDLVIGGSPCNDLSIVNPAR |
| | | KGLYEGTGRLFFEFYRLLHDARPKEGDDRPFFWLFENVVAMG |
| | | VSDKRDISRFLESNPVMIDAKEVSAAHRARYFWGNLPGMNRP |
| | | LASTVNDKLELQECLEHGRIAKFSKVRTITTRSNSIKQGKDQH |
| | | FPVFMNEKEDILWCTEMERVFGFPVHYTDVSNMSRLARQRLL |
| | | GRSWSVPVIRHLFAPLKEYFACVSSGNSNANSRGPSFSSGLVP |
| | | LSLRGSHSPLEMYKTVPVWKREPVRVLSLFGDIKKELTTLGFL |
| | | ENGSDPGRLKHLDDVTNTVRRDVEEWGPFDLVYGSTPPLGH |
| | | ACDHPPGWYLFQFHRVLQYARPRPGSPQAFFWMFVDNLVLT |
| | | EDDRAVATRFLETDPVTIQDVCGRAVRNAVHVWSNIPAVKSR |
| | | HSALFSQEESFLRAQDRQRAKPPARGPAKLVKNCFLPLREYFK |
| | | YFSTEFTSSLGGPSSGAPPPSGGSPAGSPTSTEEGTSESATPESG |
| | | PGTSTEPSEGSAPGSPAGSPTSTEEGTSTEPSEGSAPGTSTEPSE |
| | | LEDKKYSIGLAIGTNSVGWAVITDEYKVPSKKFKVLGNTDRH |
| | | SIKKNLIGALLFDSGETAEATRLKRTARRRYTRRKNRICYLQEI |
| | | FSNEMAKVDDSFFHRLEESFLVEEDKKHERHPIFGNIVDEVAY |
| | | HEKYPTIYHLRKKLVDSTDKADLRLIYLALAHMIKFRGHFLIE |
| | | GDLNPDNSDVDKLFIQLVQTYNQLFEENPINASGVDAKAILSA |
| | | RLSKSRRLENLIAQLPGEKKNGLFGNLIALSLGLTPNFKSNFDL |
| | | AEDAKLQLSKDTYDDDLDNLLAQIGDQYADLFLAAKNLSDAI |
| | | LLSDILRVNTEITKAPLSASMIKRYDEHHQDLTLLKALVRQQL |
| | | PEKYKEIFFDQSKNGYAGYIDGGASQEEFYKFIKPILEKMDGT |
| | | EELLVKLNREDLLRKQRTFDNGSIPHQIHLGELHAILRRQEDF |
| | | YPFLKDNREKIEKILTFRIPYYVGPLARGNSRFAWMTRKSEETI |
| | | TPWNFEEVVDKGASAQSFIERMTNFDKNLPNEKVLPKHSLLY |
| | | EYFTVYNELTKVKYVTEGMRKPAFLSGEQKKAIVDLLFKTNR |
| | | KVTVKQLKEDYFKKIECFDSVEISGVEDRFNASLGTYHDLLKII |
| | | KDKDFLDNEENEDILEDIVLTLTLFEDREMIEERLKTYAHLFD |
| | | DKVMKQLKRRRYTGWGRLSRKLINGIRDKQSGKTILDFLKSD |
| | | GFANRNFMQLIHDDSLTFKEDIQKAQVSGQGDSLHEHIANLA |
| | | GSPAIKKGILQTVKVVDELVKVMGRHKPENIVIEMARENQTT |
| | | QKGQKNSRERMKRIEEGIKELGSQILKEHPVENTQLQNEKLYL |
| | | YYLQNGRDMYVDQELDINRLSDYDVDAIVPQSFLKDDSIDNK |
| | | VLTRSDKNRGKSDNVPSEEVVKKMKNYWRQLLNAKLITQRK |
| | | FDNLTKAERGGLSELDKAGFIKRQLVETRQITKHVAQILDSRM |
| | | NTKYDENDKLIREVKVITLKSKLVSDFRKDFQFYKVREINNYH |
| | | HAHDAYLNAVVGTALIKKYPKLESEFVYGDYKVYDVRKMIA |
| | | KSEQEIGKATAKYFFYSNIMNFFKTEITLANGEIRKRPLIETNG |
| | | ETGEIVWDKGRDFATVRKVLSMPQVNIVKKTEVQTGGFSKES |
| | | ILPKRNSDKLIARKKDWDPKKYGGFDSPTVAYSVLVVAKVEK |
| | | GKSKKLKSVKELLGITIMERSSFEKNPIDFLEAKGYKEVKKDLI |
| | | IKLPKYSLFELENGRKRMLASAGELQKGNELALPSKYVNFLY |
| | | LASHYEKLKGSPEDNEQKQLFVEQHKHYLDEIIEQISEFSKRVI |
| | | LADANLDKVLSAYNKHRDKPIREQAENIIHLFTLTNLGAPAAF |
| | | KYFDTTIDRKRYTSTKEVLDATLIHQSITGLYETRIDLSQLGGD |
| | | SPKKKRKVGVDGSSGSETPGTSESATPESTGMNNSQGRVTFE |
| | | DVTVNFTQGEWQRLNPEQRNLYRDVMLENYSNLVSVGQGET |
| | | TKPDVILRLEQGKEPWLEEEEVLGSGRAEKNGDIGGQIWKPK |
| | | DVKESLSAKRPAATKKAGQAKKK |
| 2117 | Fusion Protein | AGGGGCGCTCGAGCAGGTTCAGAAGGAGATCAAAAACCCC |
| | Configuration 17 | CAAGGATCAAACATGAAGAGACCTGCTGCCACCAAGAAGG |
| | DNA | CCGGCCAGGCCAAGAAAAAGTACAATCACGATCAGGAGTT |
| | | CGACCCCCCTAAGGTGTACCCACCAGTGCCTGCAGAGAAG |
| | | AGGAAGCCAATCCGGGTGCTGAGCCTGTTTGATGGCATCGC |
| | | CACCGGCCTGCTGGTGCTGAAGGATCTGGGCATCCAGGTG |
| | | GACCGGTACATCGCCTCCGAGGTGTGCGAGGATTCTATCAC |
| | | CGTGGGCATGGTGCGCCACCAGGGCAAGATCATGTATGTG |
| | | GGCGACGTGCGGTCCGTGACACAGAAGCACATCCAGGAGT |
| | | GGGGCCCATTCGATCTGGTGATCGGCGGCAGCCCCTGTAAT |
| | | GACCTGTCCATCGTGAACCCTGCAAGGAAGGGACTGTACG |
| | | AGGGAACCGGCCGGCTGTTCTTTGAGTTTTATAGACTGCTG |
| | | CACGACGCCAGGCCTAAGGAGGGCGACGATAGACCATTCT |
| | | TTTGGCTGTTCGAGAATGTGGTGGCTATGGGCGTGAGCGAT |
| | | AAGAGGGACATCTCCAGGTTTCTGGAGTCTAACCCCGTGAT |
| | | GATCGATGCAAAGGAGGTGTCCGCCGCACACAGAGCCAGG |
| | | TATTTCTGGGGCAATCTGCCAGGAATGAACAGGCCACTGGC |
| | | AAGCACCGTGAATGACAAGCTGGAGCTGCAGGAGTGCCTG |
| | | GAGCACGGAAGGATCGCCAAGTTTTCCAAGGTGCGCACAA |
| | | TCACCACACGGAGCAATTCCATCAAGCAGGGCAAGGATCA |
| | | GCACTTCCCCGTGTTCATGAACGAGAAGGAGGACATCCTGT |
| | | GGTGTACCGAGATGGAGAGAGTGTTCGGCTTTCCAGTGCAC |
| | | TACACAGACGTGTCTAACATGAGCAGGCTGGCAAGGCAGC |
| | | GGCTGCTGGGCAGATCTTGGAGCGTGCCCGTGATCAGGCA |
| | | CCTGTTCGCCCCTCTGAAGGAGTATTTTGCCTGCGTGAGCA |
| | | GCGGCAACTCCAATGCCAACAGCCGGGGCCCCTCTTTCAGC |
| | | TCCGGATTGGTGCCTCTGAGCCTGAGGGGCTCCCACAGTCC |
| | | CCTTGAGATGTATAAAACTGTGCCTGTGTGGAAGAGAGAG |
| | | CCAGTGCGGGTGCTGTCCCTTTTTGGTGACATCAAGAAAGA |
| | | GCTGACGACTTTGGGCTTTCTGGAAAACGGCTCTGACCCGG |
| | | GCCGACTGAAACATTTGGACGATGTCACCAATACGGTGAG |
| | | GAGGGACGTGGAAGAATGGGGCCCGTTCGACCTCGTGTAC |
| | | GGCTCCACGCCGCCCCTCGGCCACGCCTGTGACCATCCTCC |
| | | CGGGTGGTACCTGTTCCAGTTCCACCGTGTGCTTCAGTACG |
| | | CGAGGCCCAGGCCGGGCAGCCCGCAGGCCTTCTTCTGGAT |
| | | GTTTGTGGACAACCTGGTGCTGACCGAGGATGACCGGGCT |
| | | GTAGCCACTCGCTTCCTGGAGACTGACCCGGTGACCATCCA |
| | | GGACGTCTGTGGCAGAGCTGTCCGGAACGCCGTGCACGTG |
| | | TGGAGCAACATCCCGGCCGTGAAAAGCAGGCACTCGGCCC |
| | | TGTTTTCCCAGGAGGAATCATTCCTGCGGGCTCAGGACAGG |
| | | CAGAGAGCAAAGCCCCCCGCCCGGGGGCCAGCCAAGCTGG |
| | | TGAAGAATTGTTTTCTCCCCCTGAGAGAATATTTCAAGTAT |
| | | TTTTCAACAGAATTCACTTCCTCTTTGGGAGGACCCTCCTCT |
| | | GGCGCCCCACCACCTAGCGGCGGCTCCCCTGCCGGCTCTCC |
| | | AACCAGCACAGAGGAGGGCACCAGCGAGTCCGCCACACCA |
| | | GAGTCTGGACCTGGCACCAGCACAGAGCCATCCGAGGGCT |
| | | CTGCCCCAGGCTCTCCTGCAGGCAGCCCTACCTCCACCGAA |
| | | GAGGGCACCAGCACAGAGCCTTCTGAGGGCAGCGCCCCAG |
| | | GCACCTCTACAGAGCCAAGCGAGCTCGAGGACAAGAAGTA |
| | | CAGCATCGGCCTGGCCATCGGCACCAACTCTGTGGGCTGGG |
| | | CCGTGATCACCGACGAGTACAAGGTGCCCAGCAAGAAATT |
| | | CAAGGTGCTGGGCAACACCGACCGGCACAGCATCAAGAAG |
| | | AACCTGATCGGAGCCCTGCTGTTCGACAGCGGCGAAACAG |
| | | CCGAGGCCACCCGGCTGAAGAGAACCGCCAGAAGAAGATA |
| | | CACCAGACGGAAGAACCGGATCTGCTATCTGCAAGAGATC |
| | | TTCAGCAACGAGATGGCCAAGGTGGACGACAGCTTCTTCC |
| | | ACAGACTGGAAGAGTCCTTCCTGGTGGAAGAGGATAAGAA |
| | | GCACGAGCGGCACCCCATCTTCGGCAACATCGTGGACGAG |
| | | GTGGCCTACCACGAGAAGTACCCCACCATCTACCACCTGAG |
| | | AAAGAAACTGGTGGACAGCACCGACAAGGCCGACCTGCGG |
| | | CTGATCTATCTGGCCCTGGCCCACATGATCAAGTTCCGGGG |
| | | CCACTTCCTGATCGAGGGCGACCTGAACCCCGACAACAGC |
| | | GACGTGGACAAGCTGTTCATCCAGCTGGTGCAGACCTACA |
| | | ACCAGCTGTTCGAGGAAAACCCCATCAACGCCAGCGGCGT |
| | | GGACGCCAAGGCCATCCTGTCTGCCAGACTGAGCAAGAGC |
| | | AGACGGCTGGAAAATCTGATCGCCCAGCTGCCCGGCGAGA |
| | | AGAAGAATGGCCTGTTCGGCAACCTGATTGCCCTGAGCCTG |
| | | GGCCTGACCCCCAACTTCAAGAGCAACTTCGACCTGGCCGA |
| | | GGATGCCAAACTGCAGCTGAGCAAGGACACCTACGACGAC |
| | | GACCTGGACAACCTGCTGGCCCAGATCGGCGACCAGTACG |
| | | CCGACCTGTTTCTGGCCGCCAAGAACCTGTCCGACGCCATC |
| | | CTGCTGAGCGACATCCTGAGAGTGAACACCGAGATCACCA |
| | | AGGCCCCCCTGAGCGCCTCTATGATCAAGAGATACGACGA |
| | | GCACCACCAGGACCTGACCCTGCTGAAAGCTCTCGTGCGGC |
| | | AGCAGCTGCCTGAGAAGTACAAAGAGATTTTCTTCGACCA |
| | | GAGCAAGAACGGCTACGCCGGCTACATTGACGGCGGAGCC |
| | | AGCCAGGAAGAGTTCTACAAGTTCATCAAGCCCATCCTGG |
| | | AAAAGATGGACGGCACCGAGGAACTGCTCGTGAAGCTGAA |
| | | CAGAGAGGACCTGCTGCGGAAGCAGCGGACCTTCGACAAC |
| | | GGCAGCATCCCCCACCAGATCCACCTGGGAGAGCTGCACG |
| | | CCATTCTGCGGCGGCAGGAAGATTTTTACCCATTCCTGAAG |
| | | GACAACCGGGAAAAGATCGAGAAGATCCTGACCTTCCGCA |
| | | TCCCCTACTACGTGGGCCCTCTGGCCAGGGGAAACAGCAG |
| | | ATTCGCCTGGATGACCAGAAAGAGCGAGGAAACCATCACC |
| | | CCCTGGAACTTCGAGGAAGTGGTGGACAAGGGCGCTTCCG |
| | | CCCAGAGCTTCATCGAGCGGATGACCAACTTCGATAAGAA |
| | | CCTGCCCAACGAGAAGGTGCTGCCCAAGCACAGCCTGCTG |
| | | TACGAGTACTTCACCGTGTATAACGAGCTGACCAAAGTGA |
| | | AATACGTGACCGAGGGAATGAGAAAGCCCGCCTTCCTGAG |
| | | CGGCGAGCAGAAAAAGGCCATCGTGGACCTGCTGTTCAAG |
| | | ACCAACCGGAAAGTGACCGTGAAGCAGCTGAAAGAGGACT |
| | | ACTTCAAGAAAATCGAGTGCTTCGACTCCGTGGAAATCTCC |
| | | GGCGTGGAAGATCGGTTCAACGCCTCCCTGGGCACATACC |
| | | ACGATCTGCTGAAAATTATCAAGGACAAGGACTTCCTGGA |
| | | CAATGAGGAAAACGAGGACATTCTGGAAGATATCGTGCTG |
| | | ACCCTGACACTGTTTGAGGACAGAGAGATGATCGAGGAAC |
| | | GGCTGAAAACCTATGCCCACCTGTTCGACGACAAAGTGAT |
| | | GAAGCAGCTGAAGCGGCGGAGATACACCGGCTGGGGCAGG |
| | | CTGAGCCGGAAGCTGATCAACGGCATCCGGGACAAGCAGT |
| | | CCGGCAAGACAATCCTGGATTTCCTGAAGTCCGACGGCTTC |
| | | GCCAACAGAAACTTCATGCAGCTGATCCACGACGACAGCC |
| | | TGACCTTTAAAGAGGACATCCAGAAAGCCCAGGTGTCCGG |
| | | CCAGGGCGATAGCCTGCACGAGCACATTGCCAATCTGGCC |
| | | GGCAGCCCCGCCATTAAGAAGGGCATCCTGCAGACAGTGA |
| | | AGGTGGTGGACGAGCTCGTGAAAGTGATGGGCCGGCACAA |
| | | GCCCGAGAACATCGTGATCGAAATGGCCAGAGAGAACCAG |
| | | ACCACCCAGAAGGGACAGAAGAACAGCCGCGAGAGAATG |
| | | AAGCGGATCGAAGAGGGCATCAAAGAGCTGGGCAGCCAG |
| | | ATCCTGAAAGAACACCCCGTGGAAAACACCCAGCTGCAGA |
| | | ACGAGAAGCTGTACCTGTACTACCTGCAGAATGGGCGGGA |
| | | TATGTACGTGGACCAGGAACTGGACATCAACCGGCTGTCC |
| | | GACTACGATGTGGACGCCATCGTGCCTCAGAGCTTTCTGAA |
| | | GGACGACTCCATCGACAACAAGGTGCTGACCAGAAGCGAC |
| | | AAGAACCGGGGCAAGAGCGACAACGTGCCCTCCGAAGAGG |
| | | TCGTGAAGAAGATGAAGAACTACTGGCGGCAGCTGCTGAA |
| | | CGCCAAGCTGATTACCCAGAGAAAGTTCGACAATCTGACC |
| | | AAGGCCGAGAGAGGCGGCCTGAGCGAACTGGATAAGGCCG |
| | | GCTTCATCAAGAGACAGCTGGTGGAAACCCGGCAGATCAC |
| | | AAAGCACGTGGCACAGATCCTGGACTCCCGGATGAACACT |
| | | AAGTACGACGAGAATGACAAGCTGATCCGGGAAGTGAAAG |
| | | TGATCACCCTGAAGTCCAAGCTGGTGTCCGATTTCCGGAAG |
| | | GATTTCCAGTTTTACAAAGTGCGCGAGATCAACAACTACCA |
| | | CCACGCCCACGACGCCTACCTGAACGCCGTCGTGGGAACC |
| | | GCCCTGATCAAAAAGTACCCTAAGCTGGAAAGCGAGTTCG |
| | | TGTACGGCGACTACAAGGTGTACGACGTGCGGAAGATGAT |
| | | CGCCAAGAGCGAGCAGGAAATCGGCAAGGCTACCGCCAAG |
| | | TACTTCTTCTACAGCAACATCATGAACTTTTTCAAGACCGA |
| | | GATTACCCTGGCCAACGGCGAGATCCGGAAGCGGCCTCTG |
| | | ATCGAGACAAACGGCGAAACCGGGGAGATCGTGTGGGATA |
| | | AGGGCCGGGATTTTGCCACCGTGCGGAAAGTGCTGAGCAT |
| | | GCCCCAAGTGAATATCGTGAAAAAGACCGAGGTGCAGACA |
| | | GGCGGCTTCAGCAAAGAGTCTATCCTGCCCAAGAGGAACA |
| | | GCGATAAGCTGATCGCCAGAAAGAAGGACTGGGACCCTAA |
| | | GAAGTACGGCGGCTTCGACAGCCCCACCGTGGCCTATTCTG |
| | | TGCTGGTGGTGGCCAAAGTGGAAAAGGGCAAGTCCAAGAA |
| | | ACTGAAGAGTGTGAAAGAGCTGCTGGGGATCACCATCATG |
| | | GAAAGAAGCAGCTTCGAGAAGAATCCCATCGACTTTCTGG |
| | | AAGCCAAGGGCTACAAAGAAGTGAAAAAGGACCTGATCAT |
| | | CAAGCTGCCTAAGTACTCCCTGTTCGAGCTGGAAAACGGCC |
| | | GGAAGAGAATGCTGGCCTCTGCCGGCGAACTGCAGAAGGG |
| | | AAACGAACTGGCCCTGCCCTCCAAATATGTGAACTTCCTGT |
| | | ACCTGGCCAGCCACTATGAGAAGCTGAAGGGCTCCCCCGA |
| | | GGATAATGAGCAGAAACAGCTGTTTGTGGAACAGCACAAG |
| | | CACTACCTGGACGAGATCATCGAGCAGATCAGCGAGTTCTC |
| | | CAAGAGAGTGATCCTGGCCGACGCTAATCTGGACAAAGTG |
| | | CTGTCCGCCTACAACAAGCACCGGGATAAGCCCATCAGAG |
| | | AGCAGGCCGAGAATATCATCCACCTGTTTACCCTGACCAAT |
| | | CTGGGAGCCCCTGCCGCCTTCAAGTACTTTGACACCACCAT |
| | | CGACCGGAAGAGGTACACCAGCACCAAAGAGGTGCTGGAC |
| | | GCCACCCTGATCCACCAGAGCATCACCGGCCTGTACGAGA |
| | | CACGGATCGACCTGTCTCAGCTGGGAGGCGACAGCCCCAA |
| | | GAAGAAGAGAAAGGTGGGAGTCGACGGATCCAGCGGCTCC |
| | | GAGACCCCAGGCACATCTGAGAGCGCCACCCCTGAGTCCA |
| | | CCGGTATGAACAATTCACAGGGGAGAGTGACATTCGAAGA |
| | | CGTGACCGTGAACTTCACCCAGGGAGAATGGCAGCGCTTG |
| | | AACCCAGAACAAAGGAACCTCTATCGGGACGTGATGCTGG |
| | | AAAACTACTCAAATTTGGTGAGCGTTGGGCAGGGTGAGAC |
| | | CACTAAGCCTGACGTGATCCTGAGATTGGAACAGGGCAAG |
| | | GAGCCTTGGCTCGAGGAAGAGGAAGTCCTGGGCTCAGGGA |
| | | GGGCCGAGAAAAACGGTGATATAGGAGGCCAGATATGGAA |
| | | GCCTAAGGACGTCAAGGAGAGCCTGAGCGCTAAACGTCCG |
| | | GCAGCAACCAAAAAAGCAGGTCAGGCCAAGAAAAAATGA |
| | | GGATCCTGAGTCTAGAAAAGATATATATAGGATTGAAGAT |
| | | CTCTCAGTTAAGTCTACAAAAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAGAAGAGCCTCCTGCAGG |
| | | AAGCTTGGCGTAATCATGGTCATAGCTGTTTCCTGTGTGAA |
| | | ATTGTTATCCGCTCACAATTCCACACAACATACGAGCCGGA |
| | | AGCATAAAGTGTAAAGCCTGGGGTGCCTAATGAGTGAGCT |
| | | AACTCACATTAATTGCGTTGCGCTCACTGCCCGCTTTCCAG |
| | | TCGGGAAACCTGTCGTGCCAGCTGCATTAATGAATCGGCCA |
| | | ACGCGCGGGGAGAGGCGGTTTGCGTATTGGGCCGCTTCCTC |
| | | GCTCACTGACTCGCTGCGCTCGGTCGTTCGGCTGCGGCGAG |
| | | CGGTATCAGCTCACTCAAAGGCGGTAATACGGTTATCCACA |
| | | GAATCAGGGGATAACGCAGGAAAGAACATGTGAGCAAAA |
| | | GGCCAGCAAAAGGCCAGGAACCGTAAAAAGGCCGCGTTGC |
| | | TGGCGTTTTTCCATAGGCTCCGCCCCCCTGACGAGCATCAC |
| | | AAAAATCGACGCTCAAGTCAGAGGTGGCGAAACCCGACAG |
| | | GACTATAAAGATACCAGGCGTTTCCCCCTGGAAGCTCCCTC |
| | | GTGCGCTCTCCTGTTCCGACCCTGCCGCTTACCGGATACCT |
| | | GTCCGCCTTTCTCCCTTCGGGAAGCGTGGCGCTTTCTCATA |
| | | GCTCACGCTGTAGGTATCTCAGTTCGGTGTAGGTCGTTCGC |
| | | TCCAAGCTGGGCTGTGTGCACGAACCCCCCGTTCAGCCCGA |
| | | CCGCTGCGCCTTATCCGGTAACTATCGTCTTGAGTCCAACC |
| | | CGGTAAGACACGACTTATCGCCACTGGCAGCAGCCACTGG |
| | | TAACAGGATTAGCAGAGCGAGGTATGTAGGCGGTGCTACA |
| | | GAGTTCTTGAAGTGGTGGCCTAACTACGGCTACACTAGAAG |
| | | AACAGTATTTGGTATCTGCGCTCTGCTGAAGCCAGTTACCT |
| | | TCGGAAAAAGAGTTGGTAGCTCTTGATCCGGCAAACAAAC |
| | | CACCGCTGGTAGCGGTGGTTTTTTTGTTTGCAAGCAGCAGA |
| | | TTACGCGCAGAAAAAAAGGATCTCAAGAAGATCCTTTGAT |
| | | CTTTTCTACGGGGTCTGACGCTCAGTGGAACGAAAACTCAC |
| | | GTTAAGGGATTTTGGTCATGAGATTATCAAAAAGGATCTTC |
| | | ACCTAGATCCTTTTAAATTAAAAATGAAGTTTTAAATCAAG |
| | | CCCAATCTGAATAATGTTACAACCAATTAACCAATTCTGAT |
| | | TAGAAAAACTCATCGAGCATCAAATGAAACTGCAATTTATT |
| | | CATATCAGGATTATCAATACCATATTTTTGAAAAAGCCGTT |
| | | TCTGTAATGAAGGAGAAAACTCACCGAGGCAGTTCCATAG |
| | | GATGGCAAGATCCTGGTATCGGTCTGCGATTCCGACTCGTC |
| | | CAACATCAATACAACCTATTAATTTCCCCTCGTCAAAAATA |
| | | AGGTTATCAAGTGAGAAATCACCATGAGTGACGACTGAAT |
| | | CCGGTGAGAATGGCAAAAGTTTATGCATTTCTTTCCAGACT |
| | | TGTTCAACAGGCCAGCCATTACGCTCGTCATCAAAATCACT |
| | | CGCATCAACCAAACCGTTATTCATTCGTGATTGCGCCTGAG |
| | | CGAGACGAAATACGCGATCGCTGTTAAAAGGACAATTACA |
| | | AACAGGAATCGAATGCAACCGGCGCAGGAACACTGCCAGC |
| | | GCATCAACAATATTTTCACCTGAATCAGGATATTCTTCTAA |
| | | TACCTGGAATGCTGTTTTTCCGGGGATCGCAGTGGTGAGTA |
| | | ACCATGCATCATCAGGAGTACGGATAAAATGCTTGATGGTC |
| | | GGAAGAGGCATAAATTCCGTCAGCCAGTTTAGTCTGACCAT |
| | | CTCATCTGTAACATCATTGGCAACGCTACCTTTGCCATGTTT |
| | | CAGAAACAACTCTGGCGCATCGGGCTTCCCATACAAGCGA |
| | | TAGATTGTCGCACCTGATTGCCCGACATTATCGCGAGCCCA |
| | | TTTATACCCATATAAATCAGCATCCATGTTGGAATTTAATC |
| | | GCGGCCTCGACGTTTCCCGTTGAATATGGCTCATAACACCC |
| | | CTTGTATTACTGTTTATGTAAGCAGACAGTTTTATTGTTCAT |
| | | GATGATATATTTTTATCTTGTGCAATGTAACATCAGAGATT |
| | | TTGAGACACGGGCCAGAGCTGCATCGCGCGTTTCGGTGATG |
| | | ACGGTGAAAACCTCTGACACATGCAGCTCCCGGAGACGGT |
| | | CACAGCTTGTCTGTAAGCGGATGCCGGGAGCAGACAAGCC |
| | | CGTCAGGGCGCGTCAGCGGGTGTTGGCGGGTGTCGGGGCT |
| | | GGCTTAACTATGCGGCATCAGAGCAGATTGTACTGAGAGT |
| | | GCACCATATGCGGTGTGAAATACCGCACAGATGCGTAAGG |
| | | AGAAAATACCGCATCAGGCGCCATTCGCCATTCAGGCTGC |
| | | GCAACTGTTGGGAAGGGCGATCGGTGCGGGCCTCTTCGCTA |
| | | TTACGCCAGCTGGCGAAAGGGGGATGTGCTGCAAGGCGAT |
| | | TAAGTTGGGTAACGCCAGGGTTTTCCCAGTCACGACGTTGT |
| | | AAAACGACGGCCAGTGAATTGTAATACGACTCACTATA |
| 2118 | Fusion Protein | MKRPAATKKAGQAKKKYNHDQEFDPPKVYPPVPAEKRKPIR |
| | Configuration 18 | VLSLFDGIATGLLVLKDLGIQVDRYIASEVCEDSITVGMVRHQ |
| | | GKIMYVGDVRSVTQKHIQEWGPFDLVIGGSPCNDLSIVNPAR |
| | | KGLYEGTGRLFFEFYRLLHDARPKEGDDRPFFWLFENVVAMG |
| | | VSDKRDISRFLESNPVMIDAKEVSAAHRARYFWGNLPGMNRP |
| | | LASTVNDKLELQECLEHGRIAKFSKVRTITTRSNSIKQGKDQH |
| | | FPVFMNEKEDILWCTEMERVFGFPVHYTDVSNMSRLARQRLL |
| | | GRSWSVPVIRHLFAPLKEYFACVSSGNSNANSRGPSFSSGLVP |
| | | LSLRGSHSPLEMYKTVPVWKREPVRVLSLFGDIKKELTTLGFL |
| | | ENGSDPGRLKHLDDVTNTVRRDVEEWGPFDLVYGSTPPLGH |
| | | ACDHPPGWYLFQFHRVLQYARPRPGSPQAFFWMFVDNLVLT |
| | | EDDRAVATRFLETDPVTIQDVCGRAVRNAVHVWSNIPAVKSR |
| | | HSALFSQEESFLRAQDRQRAKPPARGPAKLVKNCFLPLREYFK |
| | | YFSTEFTSSLGGPSSGAPPPSGGSPAGSPTSTEEGTSESATPESG |
| | | PGTSTEPSEGSAPGSPAGSPTSTEEGTSTEPSEGSAPGTSTEPSE |
| | | LEDKKYSIGLAIGTNSVGWAVITDEYKVPSKKFKVLGNTDRH |
| | | SIKKNLIGALLFDSGETAEATRLKRTARRRYTRRKNRICYLQEI |
| | | FSNEMAKVDDSFFHRLEESFLVEEDKKHERHPIFGNIVDEVAY |
| | | HEKYPTIYHLRKKLVDSTDKADLRLIYLALAHMIKFRGHFLIE |
| | | GDLNPDNSDVDKLFIQLVQTYNQLFEENPINASGVDAKAILSA |
| | | RLSKSRRLENLIAQLPGEKKNGLFGNLIALSLGLTPNFKSNFDL |
| | | AEDAKLQLSKDTYDDDLDNLLAQIGDQYADLFLAAKNLSDAI |
| | | LLSDILRVNTEITKAPLSASMIKRYDEHHQDLTLLKALVRQQL |
| | | PEKYKEIFFDQSKNGYAGYIDGGASQEEFYKFIKPILEKMDGT |
| | | EELLVKLNREDLLRKQRTFDNGSIPHQIHLGELHAILRRQEDF |
| | | YPFLKDNREKIEKILTFRIPYYVGPLARGNSRFAWMTRKSEETI |
| | | TPWNFEEVVDKGASAQSFIERMTNFDKNLPNEKVLPKHSLLY |
| | | EYFTVYNELTKVKYVTEGMRKPAFLSGEQKKAIVDLLFKTNR |
| | | KVTVKQLKEDYFKKIECFDSVEISGVEDRFNASLGTYHDLLKII |
| | | KDKDFLDNEENEDILEDIVLTLTLFEDREMIEERLKTYAHLFD |
| | | DKVMKQLKRRRYTGWGRLSRKLINGIRDKQSGKTILDFLKSD |
| | | GFANRNFMQLIHDDSLTFKEDIQKAQVSGQGDSLHEHIANLA |
| | | GSPAIKKGILQTVKVVDELVKVMGRHKPENIVIEMARENQTT |
| | | QKGQKNSRERMKRIEEGIKELGSQILKEHPVENTQLQNEKLYL |
| | | YYLQNGRDMYVDQELDINRLSDYDVDAIVPQSFLKDDSIDNK |
| | | VLTRSDKNRGKSDNVPSEEVVKKMKNYWRQLLNAKLITQRK |
| | | FDNLTKAERGGLSELDKAGFIKRQLVETRQITKHVAQILDSRM |
| | | NTKYDENDKLIREVKVITLKSKLVSDFRKDFQFYKVREINNYH |
| | | HAHDAYLNAVVGTALIKKYPKLESEFVYGDYKVYDVRKMIA |
| | | KSEQEIGKATAKYFFYSNIMNFFKTEITLANGEIRKRPLIETNG |
| | | ETGEIVWDKGRDFATVRKVLSMPQVNIVKKTEVQTGGFSKES |
| | | ILPKRNSDKLIARKKDWDPKKYGGFDSPTVAYSVLVVAKVEK |
| | | GKSKKLKSVKELLGITIMERSSFEKNPIDFLEAKGYKEVKKDLI |
| | | IKLPKYSLFELENGRKRMLASAGELQKGNELALPSKYVNFLY |
| | | LASHYEKLKGSPEDNEQKQLFVEQHKHYLDEIIEQISEFSKRVI |
| | | LADANLDKVLSAYNKHRDKPIREQAENIIHLFTLTNLGAPAAF |
| | | KYFDTTIDRKRYTSTKEVLDATLIHQSITGLYETRIDLSQLGGD |
| | | SPKKKRKVGVDGSSGSETPGTSESATPESTGMNNSQGRVTFE |
| | | DVTVNFTQGEWQRLNPEQRNLYRDVMLENYSNLVSVGQGET |
| | | TKPDVILRLEQGKEPWLEEEEVLGSGRAEKNGDIGGQIWKPK |
| | | DVKESLSAKRPAATKKAGQAKKK |
| 2119 | Fusion Protein | AGAAACTAGCGTAAATTCAAATATAGGTCAGGCTTCAACG |
| | Configuration 18 | TCAACAAATATGATGAAGAGACCTGCTGCCACCAAGAAGG |
| | DNA | CCGGCCAGGCCAAGAAAAAGTACAATCACGATCAGGAGTT |
| | | CGACCCCCCTAAGGTGTACCCACCAGTGCCTGCAGAGAAG |
| | | AGGAAGCCAATCCGGGTGCTGAGCCTGTTTGATGGCATCGC |
| | | CACCGGCCTGCTGGTGCTGAAGGATCTGGGCATCCAGGTG |
| | | GACCGGTACATCGCCTCCGAGGTGTGCGAGGATTCTATCAC |
| | | CGTGGGCATGGTGCGCCACCAGGGCAAGATCATGTATGTG |
| | | GGCGACGTGCGGTCCGTGACACAGAAGCACATCCAGGAGT |
| | | GGGGCCCATTCGATCTGGTGATCGGCGGCAGCCCCTGTAAT |
| | | GACCTGTCCATCGTGAACCCTGCAAGGAAGGGACTGTACG |
| | | AGGGAACCGGCCGGCTGTTCTTTGAGTTTTATAGACTGCTG |
| | | CACGACGCCAGGCCTAAGGAGGGCGACGATAGACCATTCT |
| | | TTTGGCTGTTCGAGAATGTGGTGGCTATGGGCGTGAGCGAT |
| | | AAGAGGGACATCTCCAGGTTTCTGGAGTCTAACCCCGTGAT |
| | | GATCGATGCAAAGGAGGTGTCCGCCGCACACAGAGCCAGG |
| | | TATTTCTGGGGCAATCTGCCAGGAATGAACAGGCCACTGGC |
| | | AAGCACCGTGAATGACAAGCTGGAGCTGCAGGAGTGCCTG |
| | | GAGCACGGAAGGATCGCCAAGTTTTCCAAGGTGCGCACAA |
| | | TCACCACACGGAGCAATTCCATCAAGCAGGGCAAGGATCA |
| | | GCACTTCCCCGTGTTCATGAACGAGAAGGAGGACATCCTGT |
| | | GGTGTACCGAGATGGAGAGAGTGTTCGGCTTTCCAGTGCAC |
| | | TACACAGACGTGTCTAACATGAGCAGGCTGGCAAGGCAGC |
| | | GGCTGCTGGGCAGATCTTGGAGCGTGCCCGTGATCAGGCA |
| | | CCTGTTCGCCCCTCTGAAGGAGTATTTTGCCTGCGTGAGCA |
| | | GCGGCAACTCCAATGCCAACAGCCGGGGCCCCTCTTTCAGC |
| | | TCCGGATTGGTGCCTCTGAGCCTGAGGGGCTCCCACAGTCC |
| | | CCTTGAGATGTATAAAACTGTGCCTGTGTGGAAGAGAGAG |
| | | CCAGTGCGGGTGCTGTCCCTTTTTGGTGACATCAAGAAAGA |
| | | GCTGACGACTTTGGGCTTTCTGGAAAACGGCTCTGACCCGG |
| | | GCCGACTGAAACATTTGGACGATGTCACCAATACGGTGAG |
| | | GAGGGACGTGGAAGAATGGGGCCCGTTCGACCTCGTGTAC |
| | | GGCTCCACGCCGCCCCTCGGCCACGCCTGTGACCATCCTCC |
| | | CGGGTGGTACCTGTTCCAGTTCCACCGTGTGCTTCAGTACG |
| | | CGAGGCCCAGGCCGGGCAGCCCGCAGGCCTTCTTCTGGAT |
| | | GTTTGTGGACAACCTGGTGCTGACCGAGGATGACCGGGCT |
| | | GTAGCCACTCGCTTCCTGGAGACTGACCCGGTGACCATCCA |
| | | GGACGTCTGTGGCAGAGCTGTCCGGAACGCCGTGCACGTG |
| | | TGGAGCAACATCCCGGCCGTGAAAAGCAGGCACTCGGCCC |
| | | TGTTTTCCCAGGAGGAATCATTCCTGCGGGCTCAGGACAGG |
| | | CAGAGAGCAAAGCCCCCCGCCCGGGGGCCAGCCAAGCTGG |
| | | TGAAGAATTGTTTTCTCCCCCTGAGAGAATATTTCAAGTAT |
| | | TTTTCAACAGAATTCACTTCCTCTTTGGGAGGACCCTCCTCT |
| | | GGCGCCCCACCACCTAGCGGCGGCTCCCCTGCCGGCTCTCC |
| | | AACCAGCACAGAGGAGGGCACCAGCGAGTCCGCCACACCA |
| | | GAGTCTGGACCTGGCACCAGCACAGAGCCATCCGAGGGCT |
| | | CTGCCCCAGGCTCTCCTGCAGGCAGCCCTACCTCCACCGAA |
| | | GAGGGCACCAGCACAGAGCCTTCTGAGGGCAGCGCCCCAG |
| | | GCACCTCTACAGAGCCAAGCGAGCTCGAGGACAAGAAGTA |
| | | CAGCATCGGCCTGGCCATCGGCACCAACTCTGTGGGCTGGG |
| | | CCGTGATCACCGACGAGTACAAGGTGCCCAGCAAGAAATT |
| | | CAAGGTGCTGGGCAACACCGACCGGCACAGCATCAAGAAG |
| | | AACCTGATCGGAGCCCTGCTGTTCGACAGCGGCGAAACAG |
| | | CCGAGGCCACCCGGCTGAAGAGAACCGCCAGAAGAAGATA |
| | | CACCAGACGGAAGAACCGGATCTGCTATCTGCAAGAGATC |
| | | TTCAGCAACGAGATGGCCAAGGTGGACGACAGCTTCTTCC |
| | | ACAGACTGGAAGAGTCCTTCCTGGTGGAAGAGGATAAGAA |
| | | GCACGAGCGGCACCCCATCTTCGGCAACATCGTGGACGAG |
| | | GTGGCCTACCACGAGAAGTACCCCACCATCTACCACCTGAG |
| | | AAAGAAACTGGTGGACAGCACCGACAAGGCCGACCTGCGG |
| | | CTGATCTATCTGGCCCTGGCCCACATGATCAAGTTCCGGGG |
| | | CCACTTCCTGATCGAGGGCGACCTGAACCCCGACAACAGC |
| | | GACGTGGACAAGCTGTTCATCCAGCTGGTGCAGACCTACA |
| | | ACCAGCTGTTCGAGGAAAACCCCATCAACGCCAGCGGCGT |
| | | GGACGCCAAGGCCATCCTGTCTGCCAGACTGAGCAAGAGC |
| | | AGACGGCTGGAAAATCTGATCGCCCAGCTGCCCGGCGAGA |
| | | AGAAGAATGGCCTGTTCGGCAACCTGATTGCCCTGAGCCTG |
| | | GGCCTGACCCCCAACTTCAAGAGCAACTTCGACCTGGCCGA |
| | | GGATGCCAAACTGCAGCTGAGCAAGGACACCTACGACGAC |
| | | GACCTGGACAACCTGCTGGCCCAGATCGGCGACCAGTACG |
| | | CCGACCTGTTTCTGGCCGCCAAGAACCTGTCCGACGCCATC |
| | | CTGCTGAGCGACATCCTGAGAGTGAACACCGAGATCACCA |
| | | AGGCCCCCCTGAGCGCCTCTATGATCAAGAGATACGACGA |
| | | GCACCACCAGGACCTGACCCTGCTGAAAGCTCTCGTGCGGC |
| | | AGCAGCTGCCTGAGAAGTACAAAGAGATTTTCTTCGACCA |
| | | GAGCAAGAACGGCTACGCCGGCTACATTGACGGCGGAGCC |
| | | AGCCAGGAAGAGTTCTACAAGTTCATCAAGCCCATCCTGG |
| | | AAAAGATGGACGGCACCGAGGAACTGCTCGTGAAGCTGAA |
| | | CAGAGAGGACCTGCTGCGGAAGCAGCGGACCTTCGACAAC |
| | | GGCAGCATCCCCCACCAGATCCACCTGGGAGAGCTGCACG |
| | | CCATTCTGCGGCGGCAGGAAGATTTTTACCCATTCCTGAAG |
| | | GACAACCGGGAAAAGATCGAGAAGATCCTGACCTTCCGCA |
| | | TCCCCTACTACGTGGGCCCTCTGGCCAGGGGAAACAGCAG |
| | | ATTCGCCTGGATGACCAGAAAGAGCGAGGAAACCATCACC |
| | | CCCTGGAACTTCGAGGAAGTGGTGGACAAGGGCGCTTCCG |
| | | CCCAGAGCTTCATCGAGCGGATGACCAACTTCGATAAGAA |
| | | CCTGCCCAACGAGAAGGTGCTGCCCAAGCACAGCCTGCTG |
| | | TACGAGTACTTCACCGTGTATAACGAGCTGACCAAAGTGA |
| | | AATACGTGACCGAGGGAATGAGAAAGCCCGCCTTCCTGAG |
| | | CGGCGAGCAGAAAAAGGCCATCGTGGACCTGCTGTTCAAG |
| | | ACCAACCGGAAAGTGACCGTGAAGCAGCTGAAAGAGGACT |
| | | ACTTCAAGAAAATCGAGTGCTTCGACTCCGTGGAAATCTCC |
| | | GGCGTGGAAGATCGGTTCAACGCCTCCCTGGGCACATACC |
| | | ACGATCTGCTGAAAATTATCAAGGACAAGGACTTCCTGGA |
| | | CAATGAGGAAAACGAGGACATTCTGGAAGATATCGTGCTG |
| | | ACCCTGACACTGTTTGAGGACAGAGAGATGATCGAGGAAC |
| | | GGCTGAAAACCTATGCCCACCTGTTCGACGACAAAGTGAT |
| | | GAAGCAGCTGAAGCGGCGGAGATACACCGGCTGGGGCAGG |
| | | CTGAGCCGGAAGCTGATCAACGGCATCCGGGACAAGCAGT |
| | | CCGGCAAGACAATCCTGGATTTCCTGAAGTCCGACGGCTTC |
| | | GCCAACAGAAACTTCATGCAGCTGATCCACGACGACAGCC |
| | | TGACCTTTAAAGAGGACATCCAGAAAGCCCAGGTGTCCGG |
| | | CCAGGGCGATAGCCTGCACGAGCACATTGCCAATCTGGCC |
| | | GGCAGCCCCGCCATTAAGAAGGGCATCCTGCAGACAGTGA |
| | | AGGTGGTGGACGAGCTCGTGAAAGTGATGGGCCGGCACAA |
| | | GCCCGAGAACATCGTGATCGAAATGGCCAGAGAGAACCAG |
| | | ACCACCCAGAAGGGACAGAAGAACAGCCGCGAGAGAATG |
| | | AAGCGGATCGAAGAGGGCATCAAAGAGCTGGGCAGCCAG |
| | | ATCCTGAAAGAACACCCCGTGGAAAACACCCAGCTGCAGA |
| | | ACGAGAAGCTGTACCTGTACTACCTGCAGAATGGGCGGGA |
| | | TATGTACGTGGACCAGGAACTGGACATCAACCGGCTGTCC |
| | | GACTACGATGTGGACGCCATCGTGCCTCAGAGCTTTCTGAA |
| | | GGACGACTCCATCGACAACAAGGTGCTGACCAGAAGCGAC |
| | | AAGAACCGGGGCAAGAGCGACAACGTGCCCTCCGAAGAGG |
| | | TCGTGAAGAAGATGAAGAACTACTGGCGGCAGCTGCTGAA |
| | | CGCCAAGCTGATTACCCAGAGAAAGTTCGACAATCTGACC |
| | | AAGGCCGAGAGAGGCGGCCTGAGCGAACTGGATAAGGCCG |
| | | GCTTCATCAAGAGACAGCTGGTGGAAACCCGGCAGATCAC |
| | | AAAGCACGTGGCACAGATCCTGGACTCCCGGATGAACACT |
| | | AAGTACGACGAGAATGACAAGCTGATCCGGGAAGTGAAAG |
| | | TGATCACCCTGAAGTCCAAGCTGGTGTCCGATTTCCGGAAG |
| | | GATTTCCAGTTTTACAAAGTGCGCGAGATCAACAACTACCA |
| | | CCACGCCCACGACGCCTACCTGAACGCCGTCGTGGGAACC |
| | | GCCCTGATCAAAAAGTACCCTAAGCTGGAAAGCGAGTTCG |
| | | TGTACGGCGACTACAAGGTGTACGACGTGCGGAAGATGAT |
| | | CGCCAAGAGCGAGCAGGAAATCGGCAAGGCTACCGCCAAG |
| | | TACTTCTTCTACAGCAACATCATGAACTTTTTCAAGACCGA |
| | | GATTACCCTGGCCAACGGCGAGATCCGGAAGCGGCCTCTG |
| | | ATCGAGACAAACGGCGAAACCGGGGAGATCGTGTGGGATA |
| | | AGGGCCGGGATTTTGCCACCGTGCGGAAAGTGCTGAGCAT |
| | | GCCCCAAGTGAATATCGTGAAAAAGACCGAGGTGCAGACA |
| | | GGCGGCTTCAGCAAAGAGTCTATCCTGCCCAAGAGGAACA |
| | | GCGATAAGCTGATCGCCAGAAAGAAGGACTGGGACCCTAA |
| | | GAAGTACGGCGGCTTCGACAGCCCCACCGTGGCCTATTCTG |
| | | TGCTGGTGGTGGCCAAAGTGGAAAAGGGCAAGTCCAAGAA |
| | | ACTGAAGAGTGTGAAAGAGCTGCTGGGGATCACCATCATG |
| | | GAAAGAAGCAGCTTCGAGAAGAATCCCATCGACTTTCTGG |
| | | AAGCCAAGGGCTACAAAGAAGTGAAAAAGGACCTGATCAT |
| | | CAAGCTGCCTAAGTACTCCCTGTTCGAGCTGGAAAACGGCC |
| | | GGAAGAGAATGCTGGCCTCTGCCGGCGAACTGCAGAAGGG |
| | | AAACGAACTGGCCCTGCCCTCCAAATATGTGAACTTCCTGT |
| | | ACCTGGCCAGCCACTATGAGAAGCTGAAGGGCTCCCCCGA |
| | | GGATAATGAGCAGAAACAGCTGTTTGTGGAACAGCACAAG |
| | | CACTACCTGGACGAGATCATCGAGCAGATCAGCGAGTTCTC |
| | | CAAGAGAGTGATCCTGGCCGACGCTAATCTGGACAAAGTG |
| | | CTGTCCGCCTACAACAAGCACCGGGATAAGCCCATCAGAG |
| | | AGCAGGCCGAGAATATCATCCACCTGTTTACCCTGACCAAT |
| | | CTGGGAGCCCCTGCCGCCTTCAAGTACTTTGACACCACCAT |
| | | CGACCGGAAGAGGTACACCAGCACCAAAGAGGTGCTGGAC |
| | | GCCACCCTGATCCACCAGAGCATCACCGGCCTGTACGAGA |
| | | CACGGATCGACCTGTCTCAGCTGGGAGGCGACAGCCCCAA |
| | | GAAGAAGAGAAAGGTGGGAGTCGACGGATCCAGCGGCTCC |
| | | GAGACCCCAGGCACATCTGAGAGCGCCACCCCTGAGTCCA |
| | | CCGGTATGAACAATTCACAGGGGAGAGTGACATTCGAAGA |
| | | CGTGACCGTGAACTTCACCCAGGGAGAATGGCAGCGCTTG |
| | | AACCCAGAACAAAGGAACCTCTATCGGGACGTGATGCTGG |
| | | AAAACTACTCAAATTTGGTGAGCGTTGGGCAGGGTGAGAC |
| | | CACTAAGCCTGACGTGATCCTGAGATTGGAACAGGGCAAG |
| | | GAGCCTTGGCTCGAGGAAGAGGAAGTCCTGGGCTCAGGGA |
| | | GGGCCGAGAAAAACGGTGATATAGGAGGCCAGATATGGAA |
| | | GCCTAAGGACGTCAAGGAGAGCCTGAGCGCTAAACGTCCG |
| | | GCAGCAACCAAAAAAGCAGGTCAGGCCAAGAAAAAATGA |
| | | GGATCCTGAGTCTAGAAAAGATATATATAGGATTGAAGAT |
| | | CTCTCAGTTAAGTCTACAAAAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAAAAAAAAAAAAAAAAA |
| | | AAAAAAAAAAAAAAAAAAAAAAGAAGAGCGAATTCACTG |
| | | GCCGTCGTTTTACAACGTCGTGACTGGGAAAACCCTGGCGT |
| | | TACCCAACTTAATCGCCTTGCAGCACATCCCCCTTTCGCCA |
| | | GCTGGCGTAATAGCGAAGAGGCCCGCACCGATCGCCCTTC |
| | | CCAACAGTTGCGCAGCCTGAATGGCGAATGGCGCCTGATG |
| | | CGGTATTTTCTCCTTACGCATCTGTGCGGTATTTCACACCGC |
| | | ATATGGTGCACTCTCAGTACAATCTGCTCTGATGCCGCATA |
| | | GTTAAGCCAGCCCCGACACCCGCCAACACCCGCTGACGCG |
| | | CCCTGACGGGCTTGTCTGCTCCCGGCATCCGCTTACAGACA |
| | | AGCTGTGACCGTCTCCGGGAGCTGCATGTGTCAGAGGTTTT |
| | | CACCGTCATCACCGAAACGCGCGATGCAGCTCTGGCCCGTG |
| | | TCTCAAAATCTCTGATGTTACATTGCACAAGATAAAAATAT |
| | | ATCATCATGAACAATAAAACTGTCTGCTTACATAAACAGTA |
| | | ATACAAGGGGTGTTATGAGCCATATTCAACGGGAAACGTC |
| | | GAGGCCGCGATTAAATTCCAACATGGATGCTGATTTATATG |
| | | GGTATAAATGGGCTCGCGATAATGTCGGGCAATCAGGTGC |
| | | GACAATCTATCGCTTGTATGGGAAGCCCGATGCGCCAGAGT |
| | | TGTTTCTGAAACATGGCAAAGGTAGCGTTGCCAATGATGTT |
| | | ACAGATGAGATGGTCAGACTAAACTGGCTGACGGAATTTA |
| | | TGCCTCTTCCGACCATCAAGCATTTTATCCGTACTCCTGATG |
| | | ATGCATGGTTACTCACCACTGCGATCCCCGGAAAAACAGC |
| | | ATTCCAGGTATTAGAAGAATATCCTGATTCAGGTGAAAATA |
| | | TTGTTGATGCGCTGGCAGTGTTCCTGCGCCGGTTGCATTCG |
| | | ATTCCTGTTTGTAATTGTCCTTTTAACAGCGATCGCGTATTT |
| | | CGTCTCGCTCAGGCGCAATCACGAATGAATAACGGTTTGGT |
| | | TGATGCGAGTGATTTTGATGACGAGCGTAATGGCTGGCCTG |
| | | TTGAACAAGTCTGGAAAGAAATGCATAAACTTTTGCCATTC |
| | | TCACCGGATTCAGTCGTCACTCATGGTGATTTCTCACTTGAT |
| | | AACCTTATTTTTGACGAGGGGAAATTAATAGGTTGTATTGA |
| | | TGTTGGACGAGTCGGAATCGCAGACCGATACCAGGATCTT |
| | | GCCATCCTATGGAACTGCCTCGGTGAGTTTTCTCCTTCATTA |
| | | CAGAAACGGCTTTTTCAAAAATATGGTATTGATAATCCTGA |
| | | TATGAATAAATTGCAGTTTCATTTGATGCTCGATGAGTTTTT |
| | | CTAATCAGAATTGGTTAATTGGTTGTAACATTATTCAGATT |
| | | GGGCTTGATTTAAAACTTCATTTTTAATTTAAAAGGATCTA |
| | | GGTGAAGATCCTTTTTGATAATCTCATGACCAAAATCCCTT |
| | | AACGTGAGTTTTCGTTCCACTGAGCGTCAGACCCCGTAGAA |
| | | AAGATCAAAGGATCTTCTTGAGATCCTTTTTTTCTGCGCGT |
| | | AATCTGCTGCTTGCAAACAAAAAAACCACCGCTACCAGCG |
| | | GTGGTTTGTTTGCCGGATCAAGAGCTACCAACTCTTTTTCC |
| | | GAAGGTAACTGGCTTCAGCAGAGCGCAGATACCAAATACT |
| | | GTTCTTCTAGTGTAGCCGTAGTTAGGCCACCACTTCAAGAA |
| | | CTCTGTAGCACCGCCTACATACCTCGCTCTGCTAATCCTGTT |
| | | ACCAGTGGCTGCTGCCAGTGGCGATAAGTCGTGTCTTACCG |
| | | GGTTGGACTCAAGACGATAGTTACCGGATAAGGCGCAGCG |
| | | GTCGGGCTGAACGGGGGGTTCGTGCACACAGCCCAGCTTG |
| | | GAGCGAACGACCTACACCGAACTGAGATACCTACAGCGTG |
| | | AGCTATGAGAAAGCGCCACGCTTCCCGAAGGGAGAAAGGC |
| | | GGACAGGTATCCGGTAAGCGGCAGGGTCGGAACAGGAGAG |
| | | CGCACGAGGGAGCTTCCAGGGGGAAACGCCTGGTATCTTT |
| | | ATAGTCCTGTCGGGTTTCGCCACCTCTGACTTGAGCGTCGA |
| | | TTTTTGTGATGCTCGTCAGGGGGGCGGAGCCTATGGAAAAA |
| | | CGCCAGCAACGCGGCCTTTTTACGGTTCCTGGCCTTTTGCT |
| | | GGCCTTTTGCTCACATGTTCTTTCCTGCGTTATCCCCTGATT |
| | | CTGTGGATAACCGTATTACCGCCTTTGAGTGAGCTGATACC |
| | | GCTCGCCGCAGCCGAACGACCGAGCGCAGCGAGTCAGTGA |
| | | GCGAGGAAGCGGCCCAATACGCAAACCGCCTCTCCCCGCG |
| | | CGTTGGCCGATTCATTAATGCAGCTGGCACGACAGGTTTCC |
| | | CGACTGGAAAGCGGGCAGTGAGCGCAACGCAATTAATGTG |
| | | AGTTAGCTCACTCATTAGGCACCCCAGGCTTTACACTTTAT |
| | | GCTTCCGGCTCGTATGTTGTGTGGAATTGTGAGCGGATAAC |
| | | AATTTCACACAGGAAACAGCTATGACCATGATTACGCCAA |
| | | GCTTTAATACGACTCACTATA |

What is claimed is:

1. A system for repressing transcription of two or more human genes in a human cell, optionally a human T lymphocyte or a human NK cell, comprising
a) two or more sets of fusion proteins comprising
i. a first fusion protein that collectively comprises
a first DNA methyltransferase (DNMT) domain and/or a first domain that recruits a DNMT (first recruiter domain), optionally wherein the first DNMT domain and/or the first recruiter domain comprise a first DNMT3A domain and/or a first DNMT3L domain, and optionally wherein the DNMT recruited by the first recruiter domain is DNMT3A, and
a first transcriptional repressor domain,
each of the first DNMT domain and/or the first recruiter domain, and the first transcriptional repressor domain being linked to a first DNA-binding domain that binds to a target region in one or more human genes selected from B2M, CIITA, TRAC, CD247, RFX5, RFXAP, CD3D, and CD3G, or a cis regulatory element regulating one or more of B2M, CIITA, TRAC, CD247, RFX5, RFXAP, CD3D, and CD3G, or
one or more nucleic acid molecules encoding the first fusion protein,
ii. a second fusion protein that collectively comprises
a second DNA methyltransferase (DNMT) domain and/or a second domain that recruits a DNMT (second recruiter domain), optionally wherein the second DNMT domain and/or the second recruiter domain comprise a second DNMT3A domain and/or a second DNMT3L domain, and optionally wherein the recruited second DNMT is DNMT3A, and
a second transcriptional repressor domain,
each of the second DNMT domain and/or the second recruiter domain, and the second transcriptional repressor domain being linked to a second DNA-binding domain that binds to a target region in one or more human genes selected from B2M, CIITA, TRAC, CD247, RFX5, RFXAP, CD3D, and CD3G, or a cis regulatory element regulating one or more of B2M, CIITA, TRAC, CD247, RFX5, RFXAP, CD3D, and CD3G, or
one or more nucleic acid molecules encoding the second fusion protein,
wherein the system does not generate a DNA break in the B2M, CIITA, TRAC, CD247, RFX5, RFXAP, CD3D, or CD3G gene.

2. The system of claim 1, wherein the first and/or the second DNA-binding domain each comprise a dead CRISPR Cas (dCas) domain, a ZFP domain, or a TALE domain.

3. The system of claim 2, wherein the first and/or the second DNA-binding domain comprises a dCas9 domain and the system further comprises (i) one or more guide RNAs or (ii)nucleic acid molecules coding for the one or more guide RNAs.

4. A system for repressing transcription of two or more human genes in a human cell, optionally a human T lymphocyte or a human NK cell, comprising
a) a first fusion protein comprising
a first DNMT3A domain,
a first DNMT3L domain,
a first DNA-binding domain, and
a first transcriptional repressor domain, and
b) a second fusion protein comprising
a second DNMT3A domain,
a second DNMT3L domain,
a second DNA-binding domain, and
a second transcriptional repressor domain, or
c) nucleic acid molecules encoding the first fusion protein and the second fusion protein,
wherein the system does not generate a DNA break in the two or more genes bound by the first DNA-binding domain and the second DNA-binding domain.

5. The system of claim 4, wherein the first and/or the second DNA-binding domain comprises a dead CRISPR Cas (dCas) domain, a ZFP domain, or a TALE domain.

6. The system of claim 5, wherein the first and/or the second DNA-binding domain comprises a dCas9 domain and the system further comprises (i) two or more guide RNAs or (ii)nucleic acid molecules coding for the two or more guide RNAs.

7. The system of any one of claims 2, 3, 5, and 6, wherein the first and/or the second dCas domain comprises a dCas9 sequence, optionally a sequence with at least 90% identity to SEQ ID NO: 12 or 13.

8. The system of any one of claims 1-7, wherein the first and/or the second DNA-binding domain binds to a target sequence in SEQ ID NO: 1219, 1220, 1283, 1284, 1285, 1286, 1287, 1288, 1313, or 1314.

9. The system of claim 2, 3, 5, or 6, wherein the first and/or the second ZFP domain targets a nucleotide sequence selected from SEQ ID NOs: 700-740, 741-795, or 796-856.

10. The system of any one of claims 1-9, wherein the first and/or the second DNMT3A domain comprises a sequence with at least 90% identity to SEQ ID NO: 574 or 575.

11. The system of any one of claims 1-10, wherein the first and/or the second DNMT3L domain comprises a sequence with at least 90% identity to a sequence selected from SEQ ID NOs: 578-581.

12. The system of any one of claims 1-10, wherein the first and/or the second DNMT3L domain comprises a sequence with at least 90% identity to a sequence selected from SEQ ID NOs: 582-603.

13. The system of any one of claims 1-3 and 7-9, wherein the first and/or the second DNMT domain comprises a sequence with at least 90% identity to a sequence selected from SEQ ID NOs: 601-603.

14. The system of any one of claims 1-13, wherein the first and/or the second transcriptional repressor domain comprises a sequence with at least 90% identity to a sequence selected from SEQ ID NOs: 33-570.

15. The system of any one of claims 1-13, wherein the first and/or the second transcriptional repressor domain comprises a KRAB domain derived from KOX1, ZIM3, ZFP28, or ZN627.

16. The system of claim 15, wherein the KRAB domain comprises a sequence with at least 90% identity to a sequence selected from SEQ ID NOs: 89, 116, 245, and 255.

17. The system of any one of claims 1-13, wherein the first and/or the second transcriptional repressor domain comprises a fusion of the N- and C-terminal regions of ZIM3 and KOX1 KRAB, and optionally comprises the amino acid sequence of SEQ ID NO: 571 or 572.

18. The system of any one of claims 1-13, wherein the first and/or the second transcriptional repressor domain is derived from KAP1, MECP2, HP1a/CBX5, HP1b, CBX8, CDYL2, TOX, TOX3, TOX4, EED, EZH2, RBBP4, RCOR1, or SCML2.

19. The system of any one of claims 1-18, wherein the first and the second DNA-binding domain comprises a dead CRISPR Cas (dCas) domain.

20. The system of any one of claims 1-19, wherein the first and the second fusion protein comprise an identical amino acid sequence.

21. The system of any one of claims 1-20, wherein the first and the second fusion protein consist of an identical amino acid sequence.

22. A system for repressing transcription of two or more human genes in a human cell, optionally a human T lymphocyte or a human NK cell, comprising
a) a fusion protein comprising
i) a DNA methyltransferase (DNMT) domain and/or a domain that recruits a DNMT (first recruiter domain), optionally wherein the DNMT domain and/or the first recruiter domain comprise a DNMT3A domain and/or a DNMT3L domain, and optionally wherein the DNMT recruited by the recruiter domain is DNMT3A;
ii) a transcriptional repressor domain;
iii) a DNA-binding domain comprising a dead CRISPR Cas (dCas) domain;
or one or more nucleic acid molecules encoding the fusion protein;
b) two or more gRNAs that each bind to a target region in a human gene selected from B2M, CIITA, TRAC, CD247, RFX5, RFXAP, CD3D, and CD3G, or a cis regulatory element regulating one or more of B2M, CIITA, TRAC, CD247, RFX5, RFXAP, CD3D, and CD3G,
wherein the system does not generate a DNA break in the B2M, CIITA, TRAC, CD247, RFX5, RFXAP, CD3D, or CD3G gene.

23. The system of claim 22, further comprising a third gRNA.

24. The system of claim 23, wherein the system represses transcription of B2M, RFXAP, and CD3D/G.

25. A human cell comprising the system of any one of claims 1-24, or progeny of the cell.

26. The human cell of claim 25, wherein the cell is a T lymphocyte or a NK cell.

27. A human cell modified by the system of any one of claims 1-24, or progeny of the cell, optionally wherein the cell is a T lymphocyte or a NK cell, optionally wherein the cell was modified ex vivo.

28. The human cell of any one of claims 25-27, wherein the cell further comprises a chimeric antigen receptor (CAR) directed against at least one antigen.

29. A pharmaceutical composition comprising the system of any one of claims 1-24 and a pharmaceutically acceptable excipient, optionally wherein
the composition comprises lipid nanoparticles (LNPs) comprising the system, and/or
the DNA-binding domain is a dCas domain and the LNPs further comprise one or more gRNAs.

30. A pharmaceutical composition comprising human cells of any one of claims 25-28 and a pharmaceutically acceptable excipient.

31. A method of treating a patient in need thereof, comprising administering the system of any one of claims 1-24, human cells of any one of claims 25-28, or the pharmaceutical composition of claim 26 or 27 to the patient.

32. The method of claim 31, wherein the patient has cancer or autoimmune disease.

33. The system of any one of claims 1-24, human cells of any one of claims 25-28, or the pharmaceutical composition of claim 29 or 30, for use in treating a patient in need thereof, optionally in the method of claim 31 or 32.

34. Use of the system of any one of claims 1-24 or the human cell of any one of claims 25-28 in the manufacture of a medicament for treating a patient in need thereof, optionally in the method of claim 31 or 32.

35. An epigenetically modified human cell comprising a plurality of genomic loci, wherein the human cell is of a cell type that, in its naturally-occurring (wildtype) state, is characterized by expression of at least one gene product from each one of the plurality of genomic loci, wherein each one of the genomic loci comprises two alleles, and wherein both alleles of each one of the plurality of genomic loci comprise a naturally-occurring (wildtype) genomic sequence associated with expression of the at least one functional gene product, and wherein the epigenetically modified human cell is characterized by reduced or abolished expression (as compared to a wildtype cell of the same cell type) of two or more, or of all, of the plurality of genomic loci.

36. The epigenetically modified human cell of claim 35, wherein the cell comprises an artificially hypermethylated genomic region in one allele, or in both alleles, of two or more, or of all, of the plurality of genomic loci.

37. The epigenetically modified human cell of claim 35 or claim 36, wherein the hypermethylated region is within 2 kb, 1 kb, or 500 bp of the transcription start site of two or more, or of all, of the plurality of genomic loci.

38. The epigenetically modified human cell of any one of claims 35-37, wherein the human cell is an immune effector cell.

39. The epigenetically modified human cell of any one of claims 35-38 wherein the human cell is a T lymphocyte.

40. The epigenetically modified human cell of any one of claims 35-39, wherein the human cell is a CD4+ T lymphocyte.

41. The epigenetically modified human cell of any one of claims 35-40, wherein the human cell comprises a chimeric antigen receptor (CAR).

42. The epigenetically modified human cell of any one of claims 35-41, wherein the human cell comprises an artificially hypermethylated region in two or more genomic loci selected from (a) the B2M, CIITA, TRAC, CD247, RFX5, RFXAP, CD3D, and CD3G genomic loci, and (b) a cis regulatory element regulating the expression of B2M, CIITA, TRAC, CD247, RFX5, RFXAP, CD3D, CD3G, or any combination thereof.

43. The epigenetically modified human cell of claim 42, wherein the human cell comprises an artificially hypermethylated region in (a) the B2M genomic locus, (b) the RFXAP genomic locus, and (c) the CD3D genomic locus or the CD3G genomic locus.

44. The human cell of any one of claim 42 or 43, wherein the human cell is characterized by (a) a level of expression of B2M that is at less than 50% (and optionally less than 25%, less than 10%, or less than 5%), the level of expression in a naturally-occurring (wild-Type) human cell of the same cell type; (b) a level of expression of RFXAP that is at less than 50% (and optionally less than 25%, less than 10%, or less than 5%), the level of expression in a naturally-occurring (wild-Type) human cell of the same cell type; and (c) a level of expression of B2M that is at less than 50% (and optionally less than 25%, less than 10%, or less than 5%), the level of expression in a naturally-occurring (wild-Type) human cell of the same cell type.

45. The human cell of any one of claims 42-44, wherein the human cell does not comprise an artificial mutation (optionally, wherein the artificial mutation comprises an indel, insertion, deletion, inversion, or point mutation, optionally, wherein the artificial mutation is the result of genetic engineering, e.g., via gene editing or base editing) in the B2M, CIITA, TRAC, CD247, RFX5, RFXAP, CD3D, and/or CD3G genomic loci, and in any cis regulatory element regulating the expression of B2M, CIITA, TRAC, CD247, RFX5, RFXAP, CD3D, and/or CD3G.

46. The human cell of any one of claims 25-28, and 35-45, wherein the human cell further comprises an artificial HLA-E-VL9 fusion protein, comprising an HLA-E heavy chain sequence and a leader peptide (VL9) sequence.

47. The human cell of claim 46, wherein the HLA-E-VL9 fusion protein further comprises a B2M protein sequence.

48. The human cell of claim 46 or 47, wherein the leader peptide sequence is selected from the group comprising VMAPRTLIL (SEQ ID NO: 674), VMAPRTLLL (SEQ ID NO: 675), VMAPRTLVL (SEQ ID NO: 676), VMAPRTVLL (SEQ ID NO: 677), and VMAPRTLFL (SEQ ID NO: 678).

49. A method, comprising administering the epigenetically modified human cell of any one of claims 35-48 to a subject in need thereof.

50. A pharmaceutical formulation comprising a population of a plurality of the epigenetically modified human cell of any one of claims 35-48 in a medium suitable for administration to a human subject.

51. The pharmaceutical formulation of claim 50, wherein the population comprises at least 100,000 cells, at least 200,000 cells, at least 500,000 cells, at least 1,000,000 cells, at least 2,000,000 cells, at least 5,000,000 cells, at least 10,000,000 cells, at least 20,000,000 cells, at least 50,000,000 cells, or at least 100,000,000 of the epigenetically modified human cells.

52. The pharmaceutical formulation of claim 50 or claim 51, wherein the population comprises at least 75%, at least 80%, at least 85%, at least 90%, at least 95%, at least 98%, at least 99%, or at least 99% of cells that do not express a functional gene product from two or more, or of all, of the plurality of genomic loci.

53. The epigenetically modified human cell of any one of claims 35-48, the method of claim 46, or the pharmaceutical composition of claims 50-52, wherein the epigenetically modified human cell does not express MHC class II and does not express MHC class I, optionally, wherein the epigenetically modified is an immune effector cell and wherein the epigenetically modified immune effector cell does not express CD3.
